(19) United States
(12) Patent Application Publication
(10) Pub. No.: US 2025/0270284 A1
(43) Pub. Date: Aug. 28, 2025

(54) MHC Class II Protein Constructs

(71) Applicant: Cue Biopharma, Inc., Boston, MA (US)

(72) Inventors: Ronald D. SEIDEL, III, Natick, MA (US); John F. ROSS, Arlington, MA (US); Chee Meng LOW, Boston, MA (US)

(21) Appl. No.: 18/988,409

(22) Filed: Dec. 19, 2024

Related U.S. Application Data
(63) Continuation of application No. PCT/US2023/026821, filed on Jun. 30, 2023.
(60) Provisional application No. 63/357,607, filed on Jun. 30, 2022.
(60) Provisional application No. 63/357,597, filed on Jun. 30, 2022.

(51) Int. Cl. C07K 14/74; A61K 39/00; C07K 14/415; C07K 14/47; C12N 9/12

(52) U.S. Cl. CPC C07K 14/70539; C07K 14/415; C07K 14/4748; C12N 9/1276; C12Y 207/07049; A61K 39/00; A61K 2039/605; C07K 2319/30

(57) ABSTRACT

Disclosed are MHC Class II constructs (CIICs) comprising DQ and DR gene products that can present peptide epitopes associated with cancers, allergies, autoimmune diseases (e.g., T1D and celiac disease), GVHD, HGVD, and infections to T cell receptors. The CIICs may also comprise sequences of immunomodulatory molecules (MODs) such as IL-2 or PD-L1 that can modulate receptors on the surface of T cells. CIICs are expressible at levels up to about 350 mg/I in culture, and are substantially stable to multiple freeze thaw cycles and to thermal denaturation at 42° C. The stability of CIICs and their ability to present peptide epitopes and MODs to T cells and their renders them useful as therapeutics for in vitro and in vivo methods of treating various cancers, allergies, autoimmune diseases (e.g., T1D and celiac disease), GVHD, HGVD, and infections.

This application claims the benefit of U.S. Provisional Patent Application No. 63/357,597 filed Jun. 30, 2022, and U.S. Provisional Patent Application No. 63/357,607 filed Jun. 30, 2022.

### I. Incorporation of Sequence Listing

The sequence listing in ST.26 XML format entitled 2910-17_PCT_ST26.xml, created on Jun. 30, 2023, comprising 732,878 bytes, prepared according to 37 CFR 1.822 to 1.824, and submitted concurrently with the filing of this application, is incorporated herein by reference in its entirety.

### II. Background

Major Histocompatibility Complex (MHC) proteins, also referred to in humans as Human Leukocyte Antigen (HLA) proteins, play a critical role in mammalian immune systems and are central to the adaptive immune response. The classic human MHC class II group of molecules is comprised of three different protein isotypes, HLA-DR, -DP, and -DQ, each of which is highly polymorphic. MHC molecules present fragments of larger molecules to T Cell Receptors (“TCR”) through a complex formed between an antigen-presenting cell displaying an MHC protein and a T cell displaying a TCR. MHC/HLA proteins have been of interest as immunological tools and potential therapeutics since they were first described and have continued to move toward the forefront of immunotherapy applications. While it has been possible to express Class I MHC/HLA molecules in quantities suitable for their use as therapeutics, Class II MHC/HLA proteins have proven more difficult to produce in amounts suitable for their use as therapeutics. The different isotypes require, for example different chaperones (see, e.g., J. Biol. Chem. 285(52): 40800-40808 (2010)). Difficulties expressing Class II HLA proteins in amounts suitable for their widespread adoption as therapeutics are particularly pronounced for HLA-DQ gene products such as HLA DQ2.5, which is a heteroduplex of the α and β subunits expressed by the HLA DQA1*0501 and HLA DQB1*0201 alleles.

### III. Summary

The present disclosure describes Class II MHC/HLA protein constructs (Class II Construct—“CIIC” or Class II Constructs —“CIICs”) and methods of expressing those CIICs in culture. In addition to the proteins and methods of their expression, the disclosure describes and includes methods of using the CIICs in vitro and in vivo both as research tools and therapeutically, either alone or in combination with other immunomodulatory molecules. The present disclosure further provides, and includes, CIICs that comprise a peptide epitope associated with, for example, autoimmune disorders such as Type 1 Diabetes (“T1D”) associated antigen or a peptide epitope of a celiac associated antigen (respectively, a “T1D-associated peptide epitope” and a “celiac-associated peptide epitope.”). For example, embodiments of the CIICs can be expressed at levels of at least 50 mg/liter (mg/I), and in some instances can reach about 100 mg/I, 150 mg/I, 200 mg/I, 250 mg/I or more. In addition to being expressed at such levels, embodiments of the single chain CIICs and their higher order complexes (e.g., duplexes) disclosed herein are heat and freeze thaw stable. The molecules are capable of withstanding exposure to temperatures well above that of the normal human body (37° C.), often in excess of 60° C., while also withstanding multiple freeze thaw cycles without substantial loss of protein to aggregation or denaturation.

At a minimum, the CIICs are comprised of a single polypeptide comprising a peptide epitope, a linker sequence and both MHC Class II α and β chain (subunit) sequences (collectively, a “Class II MHC protein sequence”). The Class II MHC protein sequence may be stabilized by one or more (e.g., two or more) disulfide bonds and one or more amino acid substitutions. A first type of stabilizing disulfide bond, sometimes referred to herein as a “body disulfide,” is formed between a cysteine in the N-terminal portion of the β1 domain polypeptide sequence and a cysteine in the C-terminal portion of the α1 domain polypeptide sequence. This disulfide bond may decrease protein breakdown (degradation). A body disulfide is shown schematically in FIG. 1 as a dashed line below, for example, construct A and as the dashed lines connecting the α1 and β1 domains in FIG. 20, structure A, which illustrates a homodimer comprising two CIICs joined by two disulfide bonds that link their IgG Fc domains.

A second type of stabilizing disulfide bond that may be present is sometimes referred to herein as a “linker disulfide.” A linker disulfide may stabilize the construct and/or constrain the peptide epitope, localizing it to the vicinity of the MHC binding cleft, thereby increasing the relative amount of time (residence time) the epitope spends in the binding cleft formed between the α and β chain polypeptide sequences. A linker disulfide bond is formed between a cysteine present or introduced into the linkerjoining the peptide epitope and the MHC polypeptides (the “L1” linker) and a cysteine in the MHC α subunit sequence, typically a cysteine in the C-terminal portion of the α1 domain. Linker disulfide bonds are shown schematically in FIG. 1 as a dashed line above, for example constructs E and F. The CIICs may also comprise a number of substitutions in the MHC (e.g., HLA) α1 domain sequence that, either alone or in addition to the body/linker disulfide bonds, act to stabilize the protein to thermal stress (e.g., freeze thaw and/or temperatures above 37° C.), reduce protein denaturation, and/or reduce nonspecific aggregation during cell expression or under conditions where the protein is subject to thermal stress. Such substitutions in the α1 domain may act together with linker and/or body disulfide bonds to stabilize the protein.

The single chain CIIC molecules, and their higher order complexes (e.g., duplexes), are capable of functionally engaging TCRs on the surface of T cells and, if the TCR is specific for the epitope, causing CD69 expression and/or signaling, or signaling by the Ick protein tyrosine kinase associated with CD4, resulting in the recruitment and activation of ZAP-70 protein kinase.

The Class II MHC protein sequence may be fused at its C-terminus (e.g., at the C-terminus of the α2 domain), directly or indirectly through e.g., a linker, to other polypeptides/proteins without losing the ability to present epitopes to CD4+ T cells. Fusing the single chain CIICs to polypeptides or proteins that can act as scaffolds (e.g., Ig Fc regions), transmembrane regions, MODs (to prepare MOD-containing CIICs), and/or additional peptides permits alteration of the biological response to CIICs in vitro and in vivo. Fusions to polypeptides or proteins may also alter a CIICs physical properties including, for example, its stability and serum half-life. Where proteins/polypeptides fused to the Class II MHC protein sequence can self-associate, the resulting CIIC fusion proteins can be complexed to form higher order complexes such as duplexes (see, e.g., FIG. 1 showing soluble MOD-less CIIC duplex structures H and I, membrane associated MOD-less CIICs K and M, and soluble MOD-containing CIIC duplex structures 0 and P). Where the polypeptides or proteins fused to the CIIC form multimers, the fusion proteins may also form other higher order complexes. Because higher order CIIC complexes are multivalent in the presentation epitope and any MOD sequences incorporated into them, the complexes may efficiently bind to and stimulate T cells. When fused to other polypeptides/proteins such as reporter enzymes (horseradish peroxidase), or when labeled (e.g., with radiochemical or fluorescent tags), or when immobilized upon various matrices, CIICs are useful for, among other things, identifying CD4+ T cells expressing a TCR specific for the CIICs epitope and presenting the epitope to those cells. CIICs and their fusion proteins, or labeled versions thereof, may also be used as therapeutic agents, diagnostic agents, and/or research tools. For example, where the CIIC contains, or is fused to, a polypeptide that can direct targeted cell killing (e.g., Fc polypeptide sequences that bring about Antibody-Dependent Cellular Cytotoxicity “ADCC” and/or Complement-Dependent Cytotoxicity (“CDC”)) the constructs may be used to remove CD4+ T cells whose TCR recognizes the epitope presented by the construct. MOD-containing CIICs (see, e.g., FIG. 1, structures N-0) may be used to modulate the response of CD4+ T cells specific to the epitope presented by the CIIC. For example, where a CIIC contains a MOD that stimulates T reg function, such as a wild-type or variant IL-2 and/or TGF-β (e.g., a masked TGF-β), it can suppress immune responses through the action of the T regs in an epitope specific manner. (In such instances, any Ig Fc polypeptide sequences employed would not bring about ADCC or CDC.)

### IV. Brief Description of the Drawings

FIG. 1 shows schematics of exemplary CIICs arranged with their epitope at the N-terminus. The segment marked “Scaffold” or “Scaffold/L4/Addn. Pep” may comprise one or more of a scaffold sequence (e.g., an Ig Fc), L4 linker (discussed below), and/or an additional peptide (Addn. Pep) sequence, and may comprise in addition to, or in place of any or all of those sequences, a membrane association sequence (“MAS” or “MASs” plural). The elements labeled “MOD” represent one or more MOD sequences, e.g., two or more MOD sequences that may be located in tandem. In certain embodiments, the epitopes are T1D-associated peptide epitopes or celiac-associated peptide epitopes.

Structures A to G depict some exemplary MOD-less CIICs having HLA-DQ, -DR, or -DP MHC subunit sequences. Structures H and I depict embodiments of soluble (non-membrane bound) MOD-less CIIC duplexes of the constructs depicted in, for example, structures A to G.

Structures J to M depict MOD-less CIIC constructs associated with a lipid bilayer (1) via a transmembrane aa sequence; however, amphipathic helices and sequences associated with secondary modifications (lipid or prenyl group addition) may also be employed to produce membrane associated CIICs. The membrane associated constructs may form higher order structures (e.g., duplexes) through interactions of their MASs (e.g., transmembrane domains) as in K and/or through interactions of interspecific or non-interspecific scaffold sequences (e.g., IgG CH2-CH3 domains) as in M.

Structures N to S depict exemplary soluble MOD-containing CIICs comprising non-interspecific scaffold (e.g., Ig Fc) sequences and body disulfide bonds. The elements labeled “MOD” represent one, two or more independently selected MOD sequences. Structures 0 to S form duplexes through a scaffold as in structure 0, R and S, any of which scaffolds may be an Ig Fc sequence as depicted in structures P and Q. In structures R and S the MODs are masked TGF-β sequences with the mask and TGF-β sequences located in cis. The masked TGF-β is depicted in the closed configuration (unavailable to bind cellular TGF-β receptors) in R, and depicted the open configuration (available to bind cellular TGF-β receptors) in S.

Structures T to X depict exemplary soluble MOD-containing CIIC duplexes with interspecific scaffolds (exemplified as Ig Fc based structures e.g., “KiH” structures). In structures T and U the MODs are masked TGF-β sequences with the mask and TGF-β sequences located in trans and depicted in the closed configuration in T, and the open configuration in U. Structures V and W depict CIIC duplexes having different independently selected MODs on each of the CIICs in the duplex. The “MOD*” in structure V represents one or more (e.g., two or more) MODs that are different from the “MOD” of the other CIICs. In structure W, the MOD of structure V is replaced with one or two independently selected wild-type (“wt.”) or variant IL-2 sequences represented by “IL-2/IL-2”, and MOD* is replaced by a masked TGF-β sequence shown in the open configuration.

The solid lines between the CIIC elements represent optional linker sequences that are independently selectable. The dashed lines represent potential body disulfide and linker disulfide bonds that may be present in any of the structures shown. The linker disulfide bonds are exemplified as dashed lines above structures E and F, whereas the body disulfide bonds are shown as dashed lines below, for example, structures A-D, F and G. In those structures where only a body disulfide is shown, it may be replaced by a linker disulfide.

FIGS. 2A-2H provide amino acid sequences from immunoglobulin polypeptides including their heavy chain constant regions (“Ig Fc” or “Fc”, e.g., the CH2-CH3 domain of IgG1) (SEQ ID NOs:1-13).

FIG. 2I provides the sequence from an Ig CH1 domain (SEQ ID NO: 14).

FIG. 2J provides the sequence from a human Ig-J chain (SEQ ID NO:15).

FIG. 3A provides at A the sequence from an Ig K chain (kappa chain) constant region (SEQ ID NO:16), and at B the sequence of an Ig A chain (lambda chain) constant region (SEQ ID NO:17).

FIG. 4 provides a sequence from Homo sapiens MHC DRA protein DRA*01:02 GenBank NP_061984.2 (SEQ ID NO:18). Aas 1-84=α1 domain; 85-178=α2 domain (italicized and underlined); 179-191=membrane proximal region connecting peptide (bolded); and 192-214=transmembrane domain (underlined). Positions Δ37, R44, G49, and 172 are bolded and underlined. The sequence “TKR” for linker disulfide cysteine substitution at aas 74-76, and the sequence “TPI” for body disulfide cysteine substitution at aas 80-82 are bolded and underlined. DRA*01:01 contains a Val residue at position 217 of the intracellular domain in place of the Leu in DRA*01:02.

FIG. 5 provides sequences from selected alleles of Homo sapiens MHC (HLA) DRB1 protein. The Swiss-Prot/UniProt reference (“sp”) and other database references for some of the alleles are as follows: DRB1-1 (DRB1*01:01) P04229.2 (SEQ ID NO:19); DRB1—(DRB1*01:02) (SEQ ID NO:20); (DRB1*01:03) (SEQ ID NO:21); DRB1-3 (DRB1*03:01 sp P01912.2 (SEQ ID NO:22); (DRB1*03:02) (SEQ ID NO:23); (DRB1*03:04) (SEQ ID NO:24); DRB1-4 (DRB1*04:01) sp P13760.1 (SEQ ID NO:25); DRB1*04:02 (SEQ ID NO:26); DRB1*04:03 (SEQ ID NO:27); DRB1*04:04 (SEQ ID NO:28); DRB1*04:05 (SEQ ID NO:29); DRB1*04:06 (SEQ ID NO:30); DRB1*04:08 (SEQ ID NO:31); DRB1-7 (DRB1*07:01) sp P13761.1 (SEQ ID NO:32); DRB1-8 (DRB1*08:01) sp Q30134.2 (SEQ ID NO:33); DRB1*08:02 (SEQ ID NO:34); DRB1*08:03 (SEQ ID NO:35); DRB1-9 (DRB1*09:01) sp Q9TQE0.1 (SEQ ID NO:36); DRB1-10 (DRB1*10:01) sp Q30167.2 (SEQ ID NO:37); DRB1-11 (DRB1*11:01) sp P20039.1 (SEQ ID NO:38); DRB1*11:03 (SEQ ID NO:39); DRB1*11:04 (SEQ ID NO:40); DRB1-12 (DRB1*12:01) sp Q95|E3.1 (SEQ ID NO:41); DRB1-13 (DRB1*13:01) sp Q5Y7Δ7.1 (SEQ ID NO:42); DRB1*13:03 (SEQ ID NO:43); DRB1-14 (DRB1*14:01) sp Q9GIY3.1 (SEQ ID NO:44); DRB1*14:02 (SEQ ID NO:45); DRB1*14:05 (SEQ ID NO:46); DRB1*14:06 (SEQ ID NO:47); DRB1-15 (DRB1*15:01) sp P01911 (SEQ ID NO:48); DRB1*15:02 (SEQ ID NO:49); DRB1*15:03 (SEQ ID NO:50); DRB1*15:04 (SEQ ID NO:51); DRB1*15:05 (SEQ ID NO:52); DRB1*15:06 (SEQ ID NO:53); DRB1*15:07 (SEQ ID NO:54); and DRB1-16 (DRB1*16:01) sp Q29974.1 (SEQ ID NO:55).

FIGS. 6-8 provide sequences from selected Homo sapiens MHC (HLA) DRB3, DRB4 and DRB5 proteins (SEQ ID NOs:56-61, respectively). In each of FIGS. 6 through 8, aas 1-95=β1 domain; aas 96-188=β2 domain; aas 189-198=membrane proximal region (underlined and italicized); and some locations for body disulfide cysteine substitution (aas 4-8) are bolded and underlined. References for the DR3 alleles in FIG. 6 include: DRB3*01:01 GenBank NP_072049.1, GenBank CAA23781.1 (SEQ ID NO:56); DRB3*02:01 (SEQ ID NO:57); and DRB3*03:01, GenBank AAN15205.1 (SEQ ID NO:58). References for the DR4 alleles in FIG. 7 include: DRB4*01:01 GenBank AAA36296.1 & ImMunoGeneTics (“IMGT”)/HLA Acc No: HLA00905 (SEQ ID NO:59) and DRB4*01:03 GenBank NP_068818.4 & IMGT”/HLA Acc No: HLA00908 (SEQ ID NO:60). References for the DRB5*01:01 allele in FIG. 8 include GenBank NP_002116.2 and IMGT/HLA Acc No:HLA00915 (SEQ ID NO:61).

FIG. 9 provides a sequence from Homo sapiens MHC DPA proteins DPA1*01:03 and DPA1*02:01 (SEQ ID NOs:62 and 63). Aas 1-87=α1 domain; 88-181=α2 domain (italicized and underlined); 182-194=membrane proximal region connecting peptide (bolded); and 195-216=transmembrane domain (underlined). Positions 40 (D40), 47 (H47), 52 (G52), and 75 (T75) are bolded and underlined. The sequence “IQR” for linker disulfide cysteine substitution at aas 77-79, and the sequence “TQA” for body disulfide cysteine substitution at aas 83-85 are bolded and underlined. For DPA1 allele sequence see DPA1*01:03 GenBank NP_001229453.1 and IMGT/HLA Acc No: HLA00499 (SEQ ID NO:62) and DPA1*02:01 GenBank: AAH09956.1 and IMGT/HLA Acc No: (SEQ ID NO:63).

FIG. 10 provides sequences from selected Homo sapiens MHC DPB1 proteins (SEQ ID NOs: 64-76). Aas 1−92=11 domain; aas 93−186=12 domain; aas 187-196=membrane proximal region (underlined and italicized); and some locations for body disulfide cysteine substitution (aas 4-8) are bolded and underlined. References for the DPB alleles in FIG. 10 include DPB1*01:01, IMGT/HLAAcc No: HLA00510 (SEQ ID NO:64); DPB1*02:01, IMGT/HLAAcc No: HLA00517 (SEQ ID NO:65); DPB1*03:01, IMGT/HLAAcc No: HLA00520 (SEQ ID NO:66); DPB1*04:01, IMGT/HLAAcc No: HLA00521, GenBank NP_002112.3 (SEQ ID NO:67); DPB1*04:02, IMGT/HLAAcc No: HLA00522, GenBank BBD34228.1 (SEQ ID NO:68); DPB1*06:01, IMGT/HLAAcc No: HLA00524 (SEQ ID NO:69); DPB1*09:01 (SEQ ID NO:70); DPB1*11:01, IMGT/HLA Acc No: HLA00528 (SEQ ID NO:71); DPB1*13:01 (SEQ ID NO:72); DPB1*35:01 (SEQ ID NO:73); DPB1*71:01, IMGT/HLA Acc No:HLA00590 (SEQ ID NO:74); DPB1*104:01 β chain aa sequence IMGT/HLA Acc No: HLA02046 (SEQ ID NO:75); and DPB1*141:01 beta chain aa sequence, IMGT/HLAAcc No: HLA10364 (SEQ ID NO:76).

FIG. 11 provides sequences from selected Homo sapiens MHC DQA1 proteins (SEQ ID NOs:77-87). Because some DQA1 alleles omit one aa at position 55 relative to DQA1*01:01, two different lengths are reported for aa positions beyond aa 55. As indicated in the figure, aas 1-85 or 86=α1 domain; 86 or 87-180 or 181=α2 domain (italicized and underlined); 181 or 182-193 or 194=membrane proximal region connecting peptide (bolded); and 194 or 195-216 or 217=transmembrane domain (underlined). Positions 40 (e.g., E40), 47 (e.g., C47), 52 (e.g., S52 or H52), and 74 or 75 (S75 or 175) are bolded and underlined. The sequence “IKR” for linker disulfide cysteine substitution at aas 76-78 or 77-79, and the sequence “TAA” for body disulfide cysteine substitution at aas 82-84 or 83-85 are bolded and underlined. For DQA1 allele sequence information see: DQA1*01:01 IMGT/HLAAcc No. HLA00601, GenBank: AAK11577.1 (SEQ ID NO:77); DQA1*01:02, IMGT/HLAAcc No:HLA00603, GenBank NP_002113.2 (SEQ ID NO:78); DQA1*01:03, GenBank AAU88031.1 (SEQ ID NO:79); DQA1*01:04, GenBank: AAU88004.1 (SEQ ID NO:80); DQA1*02:01, IMGT/HLAAcc No:HLA00607, NCBI PDB 6PX6_A (SEQ ID NO:81); DQA1*03:01, IMGT/HLA Acc No:HLA00609, GenBank: AAA59756.1 (SEQ ID NO:82); DQA1*03:02, GenBank: AAU88001.1 (SEQ ID NO:83); DQA1*04:01, IMGT/HLAAcc No:HLA00612, GenBank: AAA36267.1 (SEQ ID NO:84); DQA1*05:01, IMGT/HLAAcc No:HLA00613, UniProtKB/Swiss-Prot: P01909 (SEQ ID NO:85); DQA1*05:05, IMGT/HLAAcc No:HLA00619, GenBank: AAU87975.1 (SEQ ID NO:86); and DQA1*06:01, IMGT/HLAAcc No:HLA00620, GenBank: QCY59255.1 (SEQ ID NO:87).

FIG. 12 provides a sequence from Homo sapiens MHC DQA2 protein HLA DQA2*01:01, GenBank NP_064440.1 (SEQ ID NO:88) as the mature protein lacking its signal sequence. Aas 1-86=α1 domain; 87-181=α2 domain (italicized and underlined); 182-194=membrane proximal region connecting peptide (bolded); and 195-217=transmembrane domain (underlined). Positions 40 (E40), 47 (Q47), 52 (S52), and 75 (F75) are bolded and underlined. The sequence “MQR” for linker disulfide cysteine substitution at aas 77-79, and the sequence “TAA” for body disulfide cysteine substitution at aas 83-85 are bolded and underlined.

FIG. 13 provides sequences from selected Homo sapiens MHC DQB1 proteins (SEQ ID NOs:89-99). Aas 1−94=11 domain; aas 95-188=β2 domain; aas 189-198=membrane proximal region (underlined and italicized); and some locations for body disulfide cysteine substitution (aas 4-8) are bolded and underlined. References for the DQB1 alleles in FIG. 13 include: DQB1*02:01, IMGT/HLA Acc No: HLA00646, NCBI Accession NO. NP_001230891.1 (SEQ ID NO:89); DQB1*02:02, IMGT/HLAAcc No:HLA00623, NCBI Accession NO. 6PX6_B (SEQ ID NO:90); DQB1*03:01, IMGT/HLAAcc No:HLA00625, NCBI Accession NO. P01920.2 (SEQ ID NO:91); DQB1*03:02, IMGT/HLAAcc No:HLA00627, NCBI Accession NO. AAA98746.1 (SEQ ID NO:92); DQB1*03:03, IMGT/HLAAcc No:HLA00629, NCBI Accession NO. AAA59755.1(SEQ ID NO:93); DQB1*03:04, IMGT/HLAAcc No:HLA00630, NCBI Accession NO. ATY52316.1 (SEQ ID NO:94); DQB1*04:01, IMGT/HLAAcc No:HLA00636, NCBI Accession NO. CAC8953441.1 (SEQ ID NO:95); DQB1*04:02, IMGT/HLAAcc No:HLA00637, NCBI Accession NO. AAA36270.1 (SEQ ID NO:96); DQB1*05:01, IMGT/HLAAcc No:HLA00638, NCBI Accession NO. AAA59765.1 (SEQ ID NO:97); DQB1*06:01, IMGT/HLAAcc No:HLA00643, NCBI Accession NO. AXU93762.1 (SEQ ID NO:98); and DQB1*06:02, IMGT/HLA Acc No:HLA00646, NCBI Accession NO. NP_002114.3 (SEQ ID NO:99).

FIG. 14 provides sequences from selected Homo sapiens MHC DQB2 proteins (SEQ ID NOs:100 and 101). Aas 1-94=β1 domain; aas 95-187=β2 domain; aas 188-197=membrane proximal region (underlined and italicized); and some locations for body disulfide cysteine substitution (aas 4-8) are bolded and underlined. For DQB2 Isoform I ((DQB2-ISO-1) allele sequence information see GenBank NP_001287719.1 and/or UniProtKB—P05538-1 (SEQ ID NO:100), and for Isoform 2 see GenBank NP_001185787.1 and/or UniProtKB—P05538-2 (SEQ ID NO:101).

FIG. 15 shows an alignment of several MHC (HLA) gene products from the DQA1, DQA2, DRA and DPA1α subunit genes permitting corresponding amino acids between the different gene products to be identified. From top to bottom, they are SEQ ID NOs:77, 81, 85, 88, 18, and 104.

FIG. 16 shows an alignment of several MHC (HLA) gene products from the DQB1, DQB2, DRB1, DRB3, DRB4, DRB5 and DPB1 β subunit genes permitting corresponding amino acids between the different gene products to be identified. From top to bottom, they are SEQ ID NOs:89, 100, 19, 56, 25, 59, 61, and 64.

FIG. 17 provides a table showing associations of HLA Class II α lleles and haplotypes with risk of an autoimmune disease. The table also provides epitopes of autoantigens (self-epitopes) associated with a number of the diseases listed.

FIG. 18 provides the aa sequences of exemplary CIICs and control constructs. Linker sequences are bolded and italicized, scaffold (e.g., Ig Fc) sequences are underlined, and epitopes are underlined and italicized. Dashed lines connecting Cys residues represent disulfide bonds; other features are described in the text. The CIICs form duplexes when expressed by mammalian cells.

FIG. 19 shows size-based chromatographic separation of five different CIICs (3832-3836) at A. At B, FIG. 19 shows reducing and non-reducing SDS page analysis of samples of CIIC 3835 and 3836. At C, FIG. 19 shows the extended (10 day-thermal stability) test data for CIICs 3835 and 3836 measured as the unaggregated fraction of duplex CIICs (monomers of duplexed CIICs) based on size-based chromatography.

FIG. 20 shows schematics of the duplex CIIC constructs at A and the split chain Class II control construct at B that were used to assess the contribution of various substitutions on protein expression levels. The dashed lines between the IgG Fc elements represent interchain disulfide bonds. The dashed lines between the α1 and β1 domain elements represent body disulfide bonds that are present in some of the constructs tested. At C, a non-reducing coomassie blue stained SDS page gel shows the protein A purified proteins as produced by CHO cells. The arrow to the right provides the location of intact duplex CIIC molecules.

FIG. 2I provides the sequences of three different isoforms of Homo sapiens TGF-β (TGF-β1, TGF-β2, and TGF-β3) as preproproteins and the mature form of TGF-β3 along with the C77S mutant of the mature protein.

FIG. 22 provides an alignment of TGF-β isoforms 1-3 with the residues corresponding to the mature form of TGF-β2 bolded, except aa residues Lys 25, Cys 77, Ile 92, and Lys 94 of TGF-β2 and their corresponding residues in TGF-β isoforms 1 and 3 that are underlined and italicized but not bolded. References for the isoforms include TGF-β1 (NP_000651.3) SEQ ID NO:157, TGF-β1 (P01137 with P10L substitution) SEQ ID NO:158, TGF-β2 (AAA50405.1) SEQ ID NO: 159, and TGF-β3 isoform 1 (NP_001316868.1) SEQ ID NO: 160.

FIG. 23A provides the sequences of a type 1 TGF-β receptor (TβRI) and its ectodomain (SEQ ID NO:163 and SEQ ID NO:164).

FIG. 23B provides the sequences of a type 2 TGF-β receptor (TβRII), its ectodomain, and fragments of the ectodomain (SEQ ID NOs:165-172). The locations indicated in bold and underlining in the isoform B are aas F30, D32, S52, E55 and D118 of the mature polypeptide, any of which may be substituted with an aa other than that occurring in the aa sequence provided. The ectodomain fragments are based upon NCBI Ref. Seq. NP_003233.4 and UniProtKB Ref. P37173; with the ectodomain sequence corresponding to aas 49 to 159 of those sequences. The substitution at aspartic acid “D119” of the mature protein with an alanine “A” (bolded, italicized, and underlined) is marked as a “D118A” substitution for consistency with the literature describing that substitution when the signal peptide is understood to be 23 aas in length as opposed to 22 aas in the NCBI record. The aa D119 numbering assignment is based on the mature protein, and accordingly, it is D141 of the precursor protein when the 22 aa signal sequence is included. The location of D32, sometimes substituted with asparagine (D32N), corresponds to D55 in the precursor protein. The corresponding aas in mature isoform A lacking its signal sequence are F55, D57, S77, E80, and D143 (see e.g., Construct 4065, SEQ ID NO:136).

FIG. 23C provides the sequences of type 3 TGF-β receptor (TβRIII) isoforms A and B. (SEQ ID NOs: 173-174).

FIG. 24 at A depicts the response of SKW-3 cells that express TCR #16S specific to the peptide epitope in construct 4214, but not the peptide epitope in construct 4149 as measured by CD69 expression. At B FIG. 24 shows the response of SKW-3 cells expressing either TCR380 or TCR #16S to Raji cells previously exposed to and presenting constructs 4149, 4062, or 4214, along with controls as measured by CD69 expression. 4149 on SKW-3 cells expressing TCR #380, confirming that construct does not activate the SKW-3 cells.

FIG. 25 provides a table showing examples of HLA Class II α lleles, MODs, and T1D-epitopes that may be incorporated into a CIIC for T1D therapy.

FIG. 26 provides the aa sequences of additional exemplary CIICs and control constructs. Linker sequences are bolded and italicized, scaffold (e.g., Ig Fc) sequences are underlined, and epitopes are underlined and italicized. Dashed lines connecting Cys residues represent disulfide bonds; other features are described in the text. The CIICs form duplexes when expressed by mammalian cells.

FIG. 27 shows at A a histogram of protein production levels in mg per liter for samples 1-20 of Example 8. At B, the figure provides chromatograms for constructs 3940 (sample 1), 3949 (sample 10), 3951 (sample 12), 3956 (sample 17), 3957 (sample 18), and a control construct 3836 (sample 20).

FIG. 28 shows a histogram of CIIC protein production levels in mg per liter for samples 1-18 of ala, α2, and w gliaden epitopes (see Example 9). In the figure, the expression level of a control CIIC with a surrogate epitope is shown as sample “C.” Bars in the histogram indicate a native epitope, and the horizontally hashed bars in the histogram (samples 3-6 and 12-14) indicate anchor-modified variants.

### V. Definitions

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically, or biochemically modified, non-natural, or derivatized nucleotide bases.

The terms “polypeptide” and “protein” are used interchangeably herein, and refer to a polymeric form of amino acids which, unless stated otherwise, are the naturally occurring proteinogenic L-amino acids that are incorporated biosynthetically into proteins during translation in a mammalian cell. Furthermore, as used herein, a “polypeptide” or “protein” includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to polymerase chain reaction (PCR) amplification or other recombinant DNA methods. References to a specific residue or residue number in a known polypeptide, e.g., position 72 or 75 of human DRA MHC class II polypeptide, are understood to refer to the amino acid at that position in the wild-type polypeptide (i.e., 172 or K75). To the extent that the sequence of the wild-type polypeptide is altered, either by addition or deletion of one or more amino acids, the specific residue or residue number will refer to the same specific amino acid in the altered polypeptide (e.g., in the addition of one amino acid at the N-terminus of a peptide reference as position 172, will be understood to indicate the amino acid, lie, that is now position 73). Substitution of an amino acid at a specific position is denoted by an abbreviation comprising, in order, the original amino acid, the position number, and the substituted amino acid, e.g., substituting the lie at position 72 with a cysteine is denoted as I72C.

A nucleic acid or polypeptide has a certain percent “sequence identity” to another nucleic acid or polypeptide, meaning that, when aligned, that percentage of nucleotides or amino acids are the same, and in the same relative position, when comparing the two sequences. Unless stated otherwise, to determine sequence identity the sequences are aligned using the computer program BLAST (BLAST+2.10.0 using default parameters), which is available over the world wide web at sites including blast.ncbi.nlm.nih.gov/Blast.cgi for BLAST+2.10.0. Unless stated otherwise, for determining positions of corresponding amino acids (e.g., when making specific substitutions), sequence comparisons are conducted using Clustal Omega Version 1.2.2 (using default parameters) available on the internet at www.ebi.ac.uk/Tools/msa/clustalo/. Where a polypeptide sequence comprises fewer aas or more aas than a reference sequence having a SEQ ID NO, the percent sequence identity of the polypeptide sequence to the reference SEQ ID NO sequence is determined by aligning and comparing the amino acids of the polypeptide sequence in the same relative position as the aas in the reference SEQ ID NO, without reference to the additional aas in the reference SEQ ID NO (where the reference SEQ ID NO has more aas than the polypeptide sequence) or the additional aas in the polypeptide sequence (where the polypeptide sequence has more aas than the reference SEQ ID NO). For example, as discussed below, a DRA α1 domain sequence may have a percent sequence identity to SEQ ID NO:102. The percent sequence identity of the DRA α1 domain sequence to SEQ ID NO:102 is determined by aligning and comparing the aas in the DRA α1 domain sequence with their corresponding aas in SEQ ID NO:102, i.e., the amino acids of the DRA α1 domain sequence in the same relative position as the aas in the reference SEQ ID NO:102. If the DRA α1 domain sequence has more aas than SEQ ID NO:102, then only the aas in the DRA α1 domain sequence that have the same relative position as the aas in SEQ ID NO:102 are considered in determining percent sequence identity and the additional aas in the DRA α1 domain sequence are not included in determining the percent identity of the DRA α1 domain sequence to SEQ ID NO:102. Similarly, if SEQ ID NO:102 has more aas than the DRA α1 domain sequence, then only the aas in SEQ ID NO: 102 that have the same relative position as the aas in the DRA α1 domain sequence are considered in determining the percent identity of the DRA α1 domain sequence to SEQ ID NO:102, and the additional aas in the SEQ ID NO:102 are not included in determining the percent identity.

As used herein amino acid (“aa” singular or “aas” plural) means the naturally occurring proteogenic amino acids incorporated into polypeptides and proteins in mammalian cell translation. Unless stated otherwise, these are: L (Leu, leucine), A (Ala, alanine), G (Gly, glycine), S (Ser, serine), V (Val, valine), F (Phe, phenylalanine), Y (Tyr, tyrosine), H (His, histidine), R (Arg, arginine), N (Asn, asparagine), E (Glu, glutamic acid), D (Asp, asparagine), C (Cys, cysteine), Q (Gln, glutamine), I (Ile, isoleucine), M (Met, methionine), P (Pro, proline), T (Thr, threonine), K (Lys, lysine), and W (Trp, tryptophan). Amino acids also include the amino acids hydroxyproline and selenocysteine, which appear in some proteins found in mammalian cells; however, unless their presence is expressly indicated they are not understood to be included.

A “conservative amino acid substitution” refers to the interchangeability in proteins of aa residues having similar side chains. For example, a group of aas having aliphatic side chains consists of glycine, alanine, valine, leucine, and isoleucine; a group of aas having aliphatic-hydroxyl side chains consists of serine and threonine; a group of aas having amide containing side chains consists of asparagine and glutamine; a group of aas having aromatic side chains consists of phenylalanine, tyrosine, and tryptophan; a group of aas having basic side chains consists of lysine, arginine, and histidine; a group of aas having acidic side chains consists of glutamate and aspartate; and a group of aas having sulfur containing side chains consists of cysteine and methionine. Exemplary conservative aa substitution groups are: valine-leucine-isoleucine, phenylalanine-tyrosine, lysine-arginine, alanine-valine-glycine, and asparagine-glutamine.

As used herein the term “in vivo” refers to any process or procedure occurring inside of the body, e.g., of a patient.

As used herein, “in vitro” refers to any process or procedure occurring outside of the body.

The term “binding” refers to a direct association between molecules and/or atoms, due to, for example, covalent, electrostatic, hydrophobic, and ionic and/or hydrogen-bond interactions, including interactions such as salt bridges and water bridges. “Covalent bonding,” or “covalent binding” as used herein, refers to the formation of one or more covalent chemical bonds between two different molecules. The term “binding,” as used with reference to the interaction between a CIIC and a T cell receptor (TCR) on a T cell, refers to a non-covalent interaction between the CIIC and TCR.

“Affinity” as used herein generally refers to the strength of non-covalent binding, increased binding affinity being correlated with a lower KD or Kd. As used herein, the term “affinity” may be described by the dissociation constant (KD or Kd) for the reversible binding of two agents (e.g., an antibody and an antigen). As used herein, the term “avidity” refers to the resistance of a complex of two or more agents to dissociation after dilution.

“T cell” includes all types of immune cells expressing CD3, including T-helper cells (CD4+T-helper cells), cytotoxic T cells (CD8+ cells), T-regulatory cells (T reg), and NK-T cells.

The term “immunomodulatory polypeptide” (also referred to as a “MOD”), as used herein, includes a wild-type or variant of a polypeptide or portion thereof that can specifically bind a cognate co-immunomodulatory polypeptide (“co-MOD” e.g., a receptor upon which it may act as an agonist or antagonist) present on a T cell, and provide a modulatory signal to the T cell when the TCR of the T cell is engaged with an MHC-epitope moiety that is specific for the TCR. Unless stated otherwise the term “MOD” includes wild-type and/or variant immunomodulatory polypeptides, and statements including reference to both wild-type and variant MODs are made to emphasize that one, the other, or both are being referenced. The signal provided by the MOD engaging its co-MOD mediates (e.g., directs) a T cell response. Such responses include, but are not limited to, proliferation, activation, differentiation, suppression/inhibition of proliferation, activation and/or differentiation, and the like.

Class II MHC protein Construct (CIIC) as used herein can be singular or plural; where required CIICs refer to the plural. CIIC and CIICs include higher order complexes of CIICs including duplexes, triplexes, etc. CIICs may be MOD-less or MOD-containing. MOD-less CIICs do not comprise an aa sequence (polypeptide sequence) of a MOD. In contrast, MOD-containing CIICs comprise all or part of the aa sequence (polypeptide sequence) of at least one (e.g., at least two) MOD.

As used herein “higher order complexes” of CIICs include, but are not limited to, CIIC complexes comprising: two (duplexes), three (triplexes), four (quadraplexes), five (pentaplexes), six (hexaplexes) CIICs, or more than six CIICs. Recitations such as “CIICs and higher order complexes thereof (duplexes)” do not change the scope of CIIC as used herein, but instead are made, for example, to emphasize that a singular CIIC or its higher order complexes are contemplated, and/or for antecedent basis.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides to provide a synthetic nucleic acid which is capable of being expressed from a recombinant transcriptional unit contained in a cell or in a cell-free transcription and translation system.

The terms “recombinant expression vector” and “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

The terms “treatment,” “treating” and the like are used herein to generally mean the use of a therapeutic agent for the purpose of obtaining one or more desired pharmacologic and/or physiologic effects. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; and/or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient” are used interchangeably herein and refer to any mammalian subject for whom diagnosis, treatment, or therapy is desired. Mammals include humans and non-human primates, and in addition include rodents (e.g., rats; mice), lagomorphs (e.g., rabbits), ungulates (e.g., cows, sheep, pigs, horses, goats, and the like), felines, canines, etc.

Unless indicated otherwise, the term “substantially” is intended to encompass both “wholly” and “largely but not wholly.” For example, an Ig Fc that “substantially does not induce cell lysis” means an Ig Fc that induces no cell lysis at all or that largely but not wholly induces no cell lysis.

As used herein, the term “about” used in connection with an amount indicates that the amount can vary by 10%. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

The terms “purifying,” “isolating,” and the like refer to the removal of a desired substance, e.g., a CIIC, from a solution containing undesired substances, e.g., contaminates, or the removal of undesired substances from a solution containing a desired substance, leaving behind essentially only the desired substance. In some instances, a purified substance may be essentially free of other substances, e.g., contaminates. As will be understood by those of skill in the art, generally, components of the solution itself, e.g., water or buffer, or salts are not considered when determining the purity of a substance.

Where a range of values is provided, it is understood that each intervening value between the upper and lower limit of that range to a tenth of the lower limit of the range is encompassed within the disclosure along with any other stated or intervening value in the range.

Unless defined otherwise, all technical and scientific terms used herein have the same meaning as commonly understood by one of ordinary skill in the art to which this disclosure belongs.

It must be noted that, as used herein and in the appended claims, the singular forms “a,” “an,” and “the” include plural referents unless the context clearly dictates otherwise. Thus, for example, reference to “a T reg” includes a plurality of such T regs and reference to “the MHC Class II α lpha chain” includes reference to one or more MHC Class II α lpha chains and equivalents thereof known to those skilled in the art, and so forth. It is further noted that the claims may be drafted to exclude any optional element. This statement is intended to serve as antecedent basis for use of such exclusive terminology as “solely,” “only” and the like in connection with the recitation of claim elements, as well as use of a “negative” limitation.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### VI. Description

### A. Class II Protein Constructs

The expression of MHC Class II proteins generally results in the poor yield of proteins and/or protein that is aggregated or relatively unstable. Among the most problematic Class II proteins to express in an active form at favorable levels are some of the DQ alleles associated with immune disorders. MHC Class II proteins such as the HLA DQ 2.5 heterodimer (comprised of the HLA α and β subunits DQA1*05:01 and DQB1*02:01) that appear to have weak interactions between the α and β subunits and/or weak associations with peptide epitopes are particularly problematic. The presence of such weak interactions, either alone or in combination, result in events such as the binding pocket collapsing (e.g., around the P1 binding region), and the subsequent irreversible aggregation and/or breakdown of the protein during cellular expression. Even with relatively stable Class II molecules that form compact Class II heterodimers of the α and β subunit sequences, such as some DR heterodimers that have relatively compact epitope binding pockets stabilized by significant stabilizing hydrogen bonds with the epitope (e.g., at P1, P4, P6/7, and P9), the residue interactions near the P1 pocket disproportionately stabilize the DR heterodimer. As a result, expression of DR protein constructs is highly dependent on the sequence of the peptide epitopes (e.g., fused to one of the HLA subunits) that can occupy and stabilize the heterodimer.

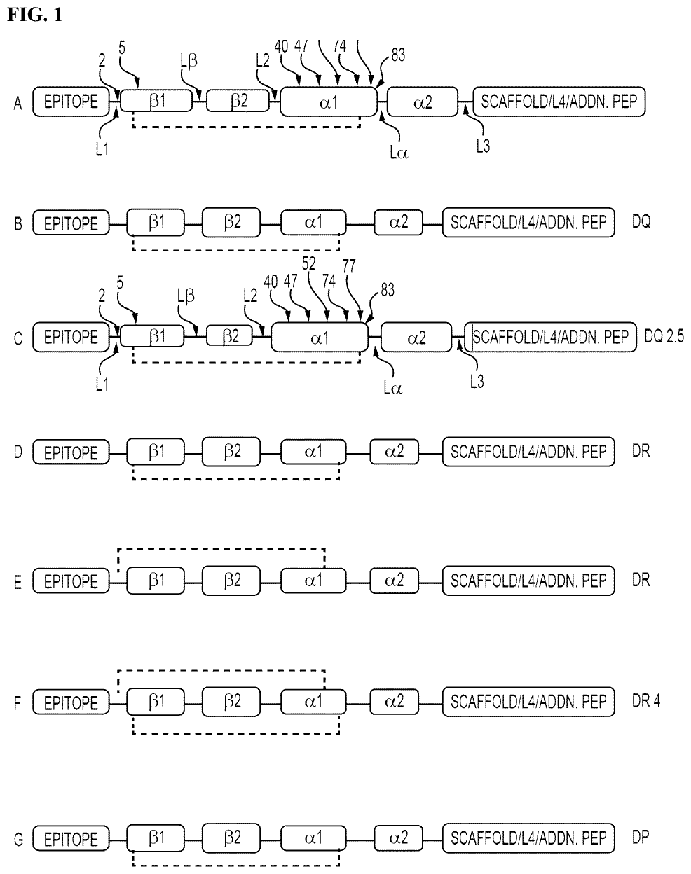

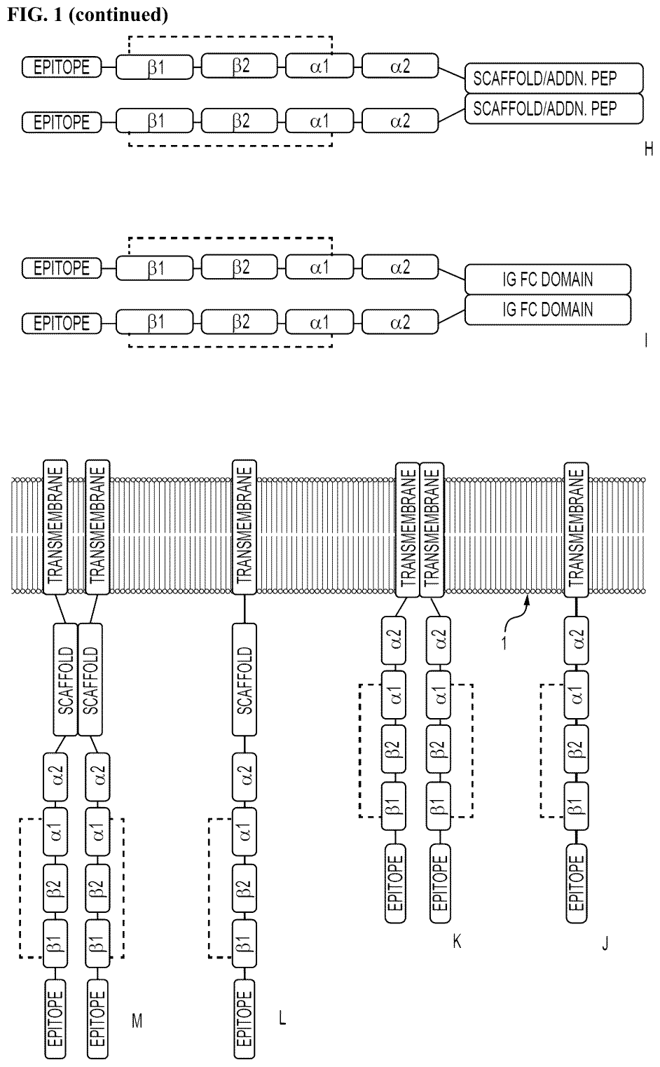

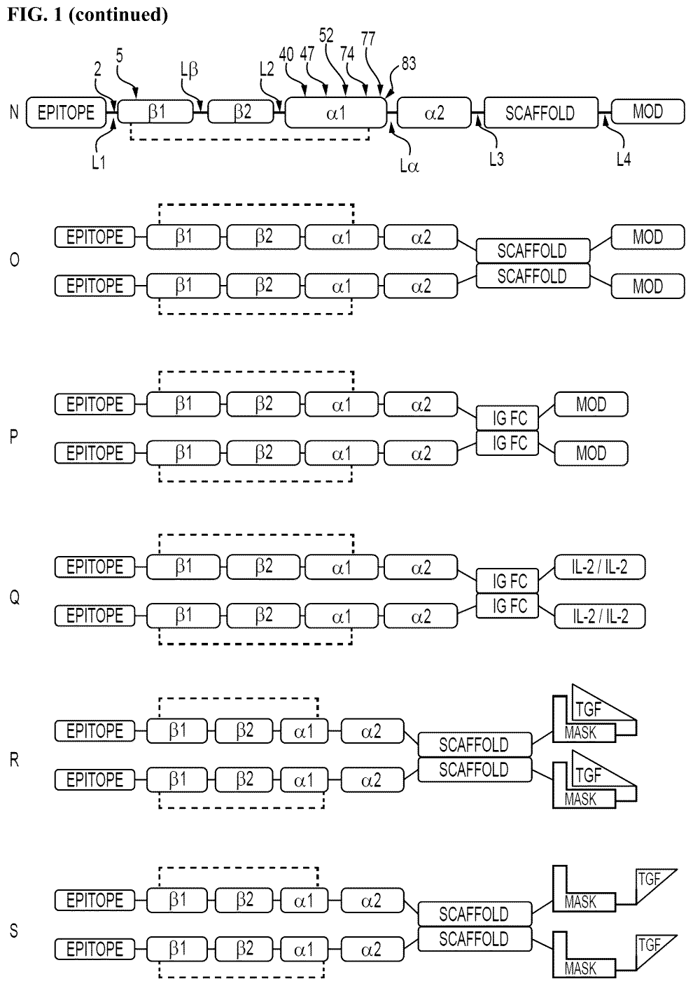

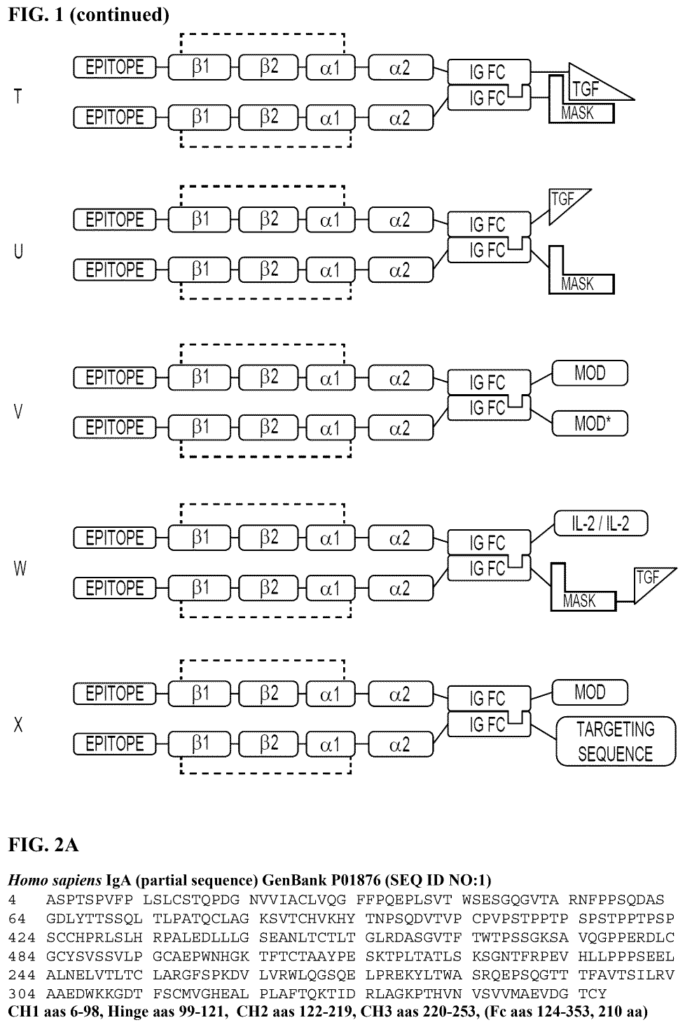

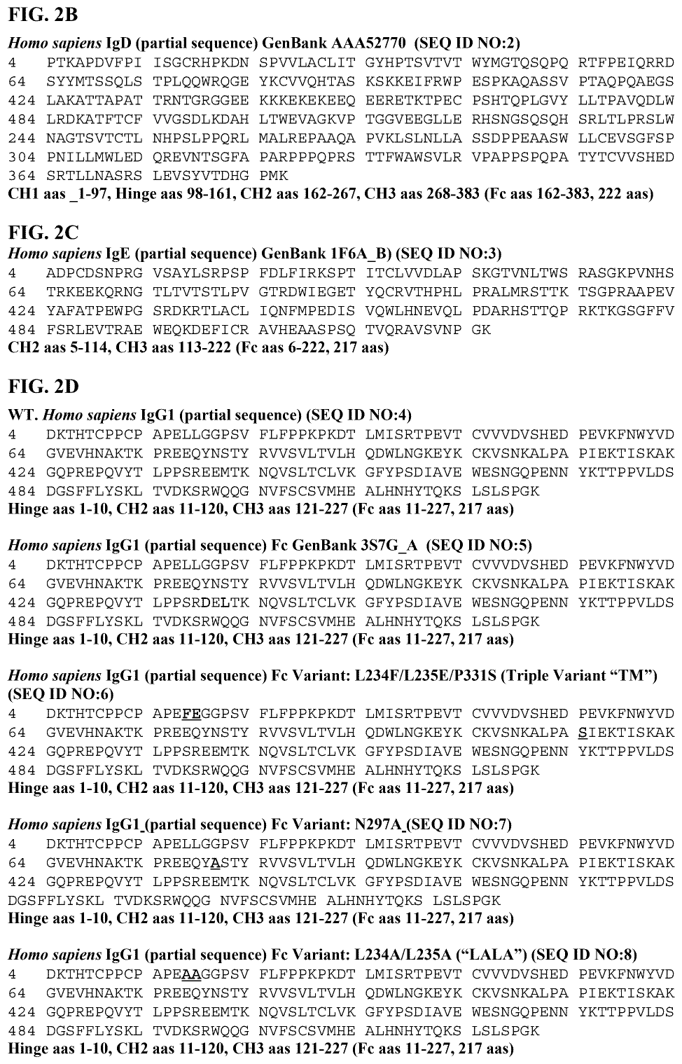

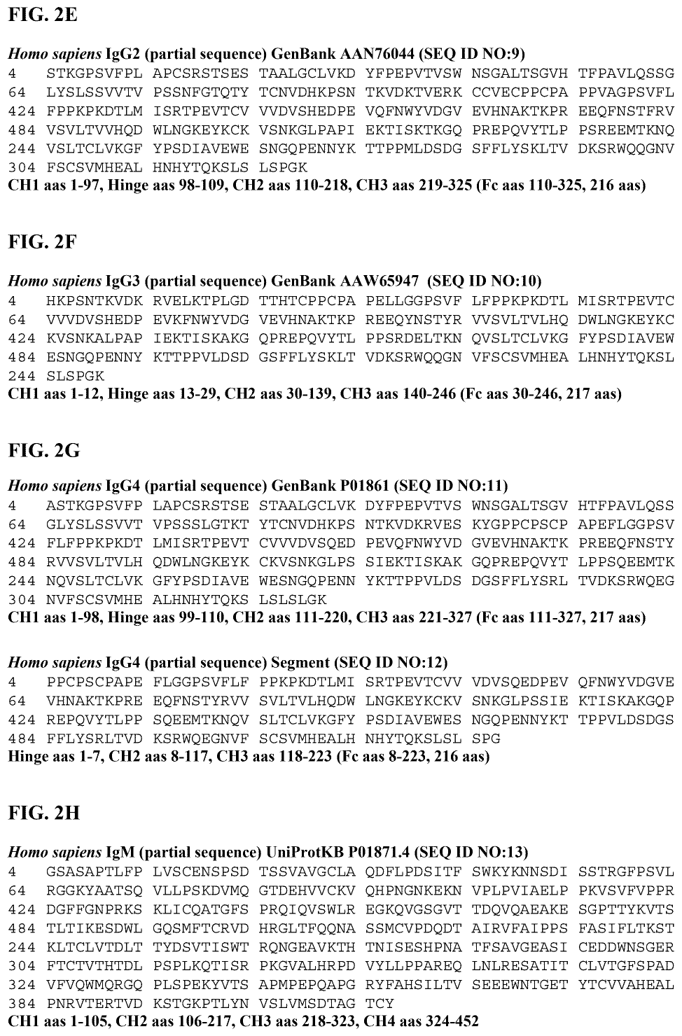

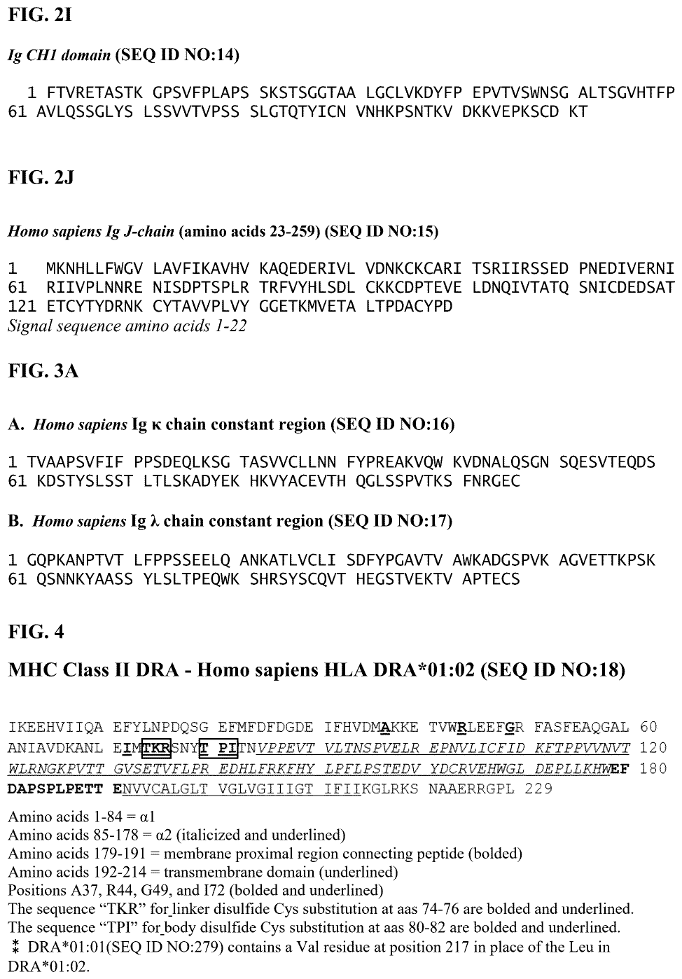

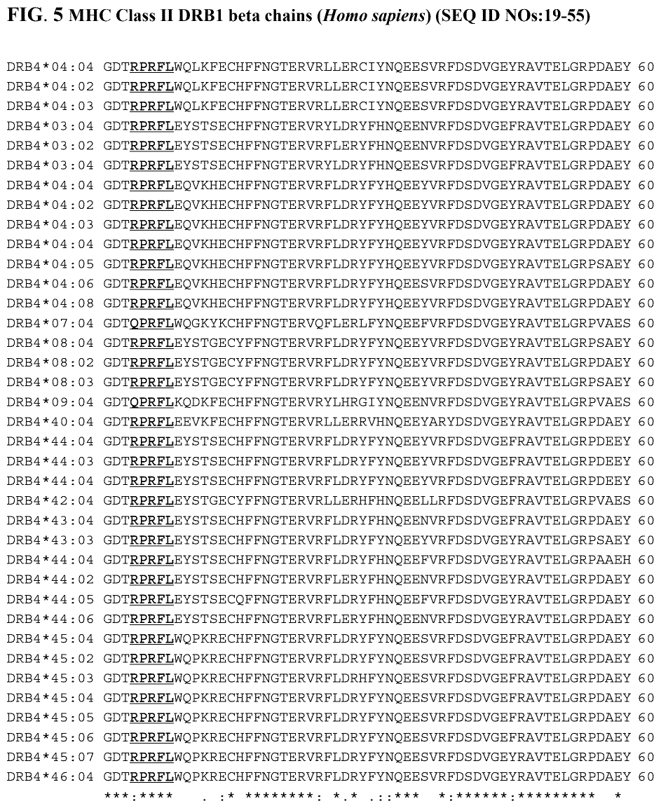

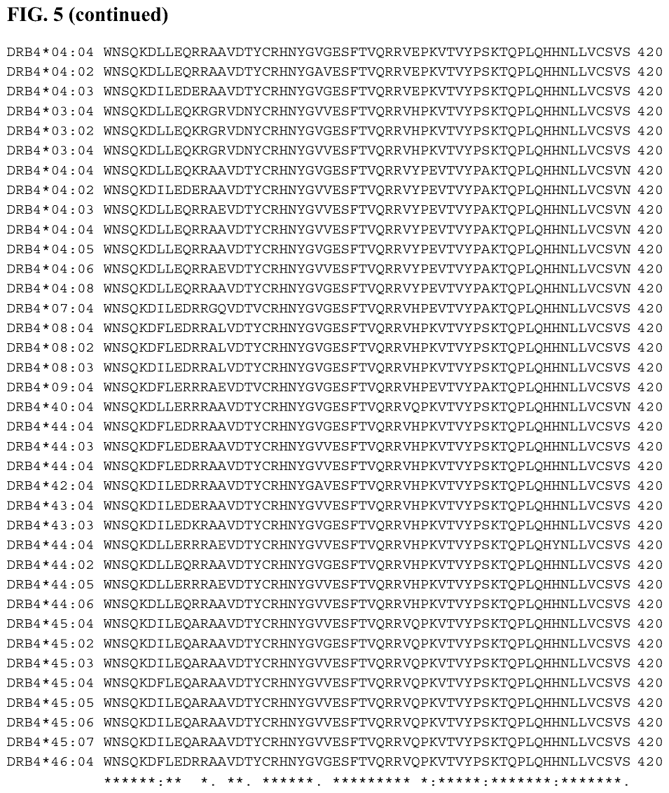

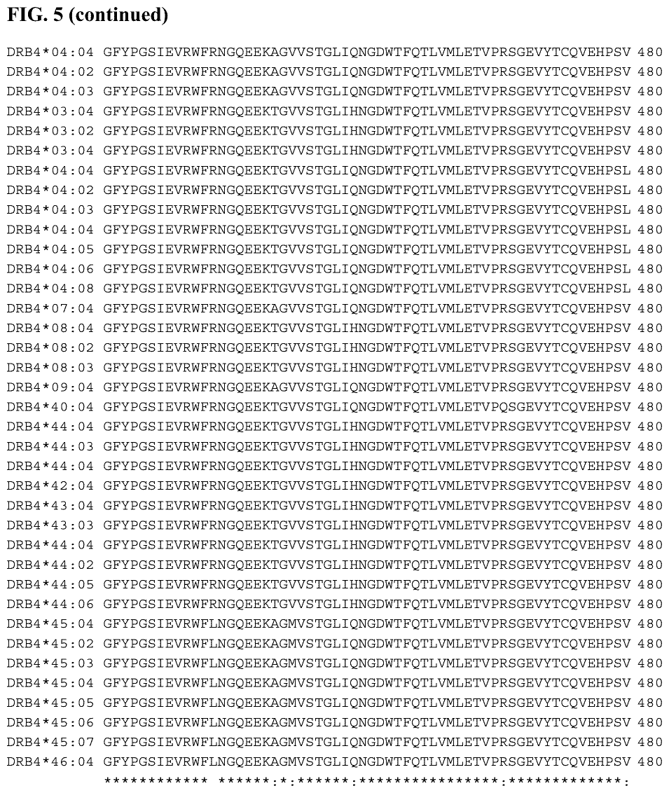

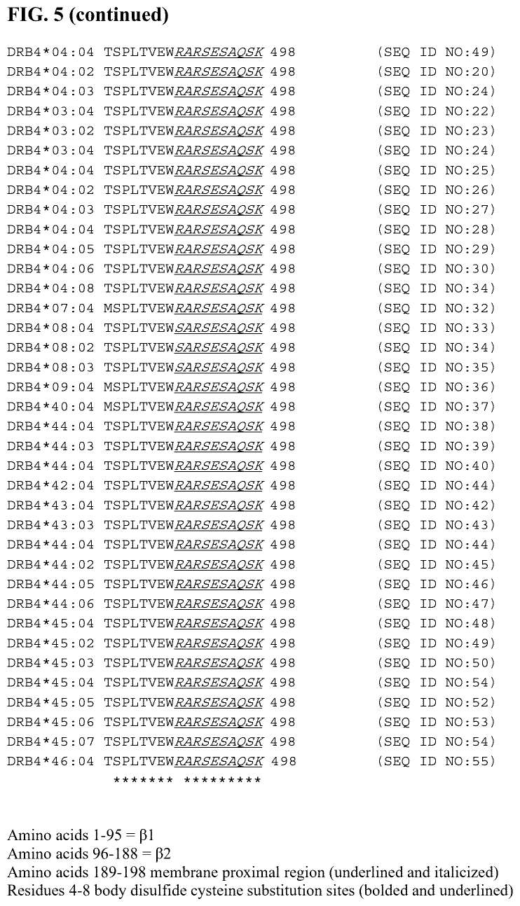

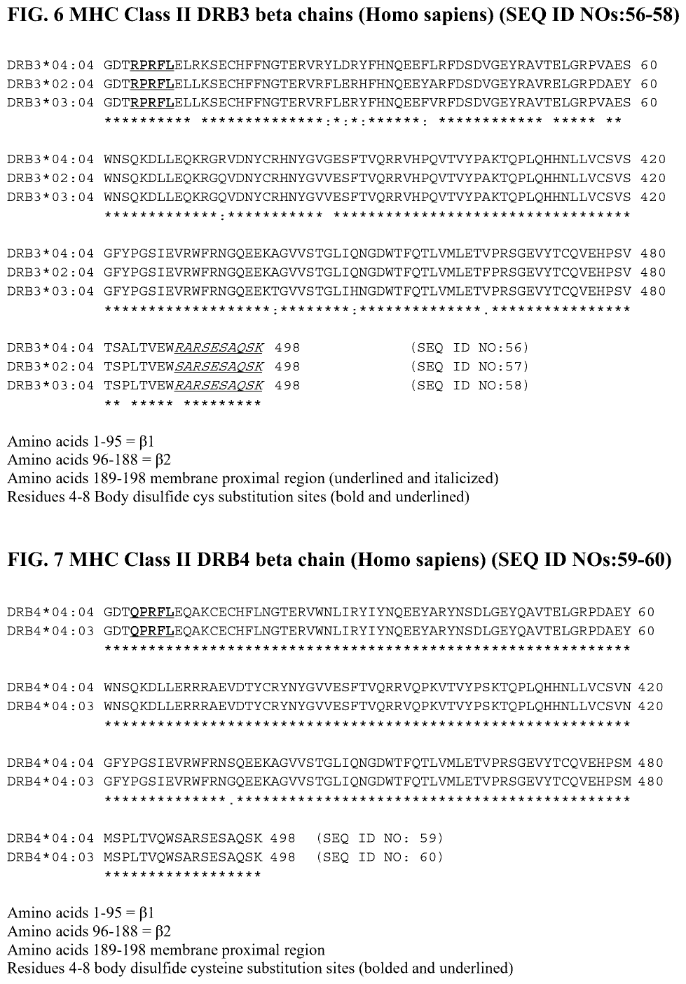

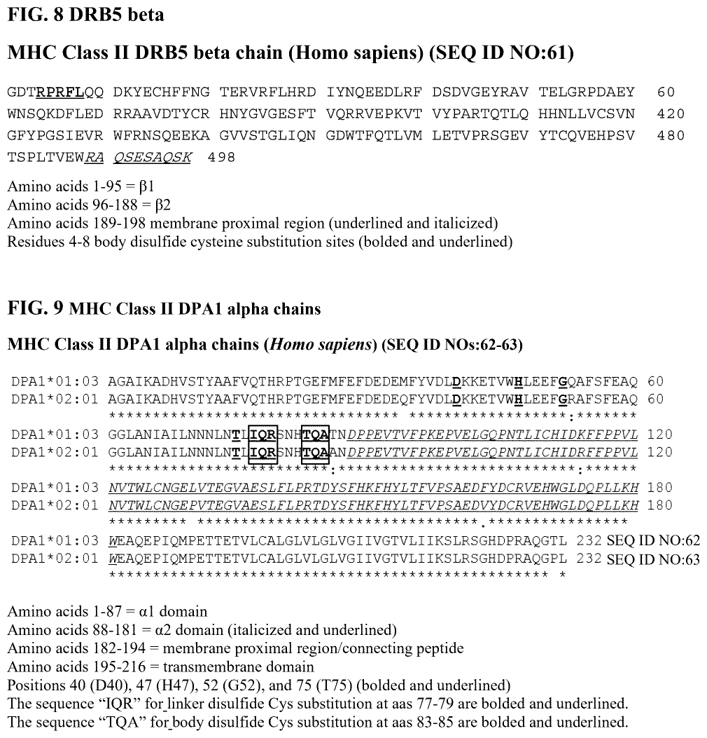

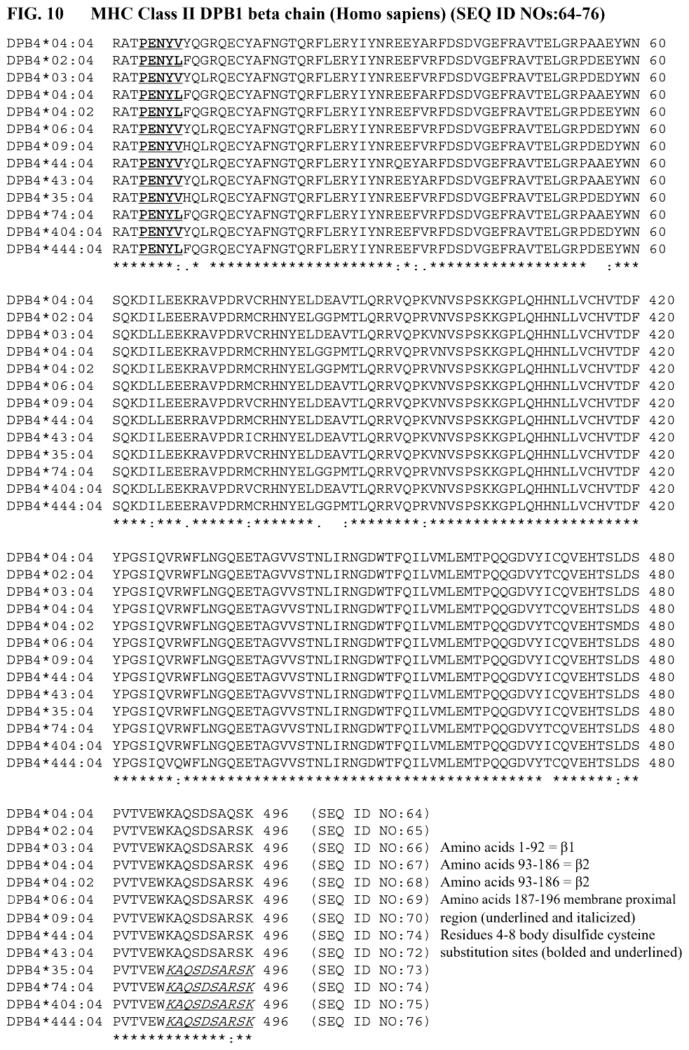

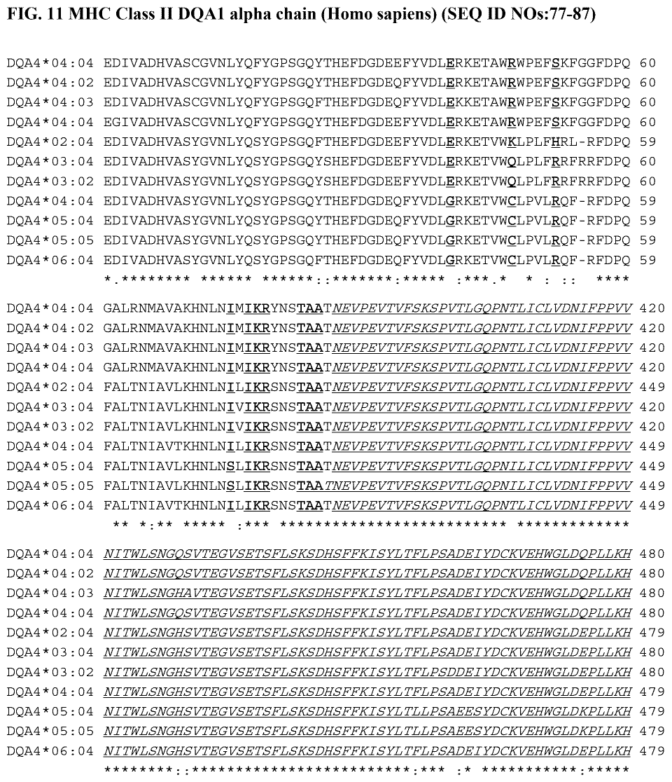

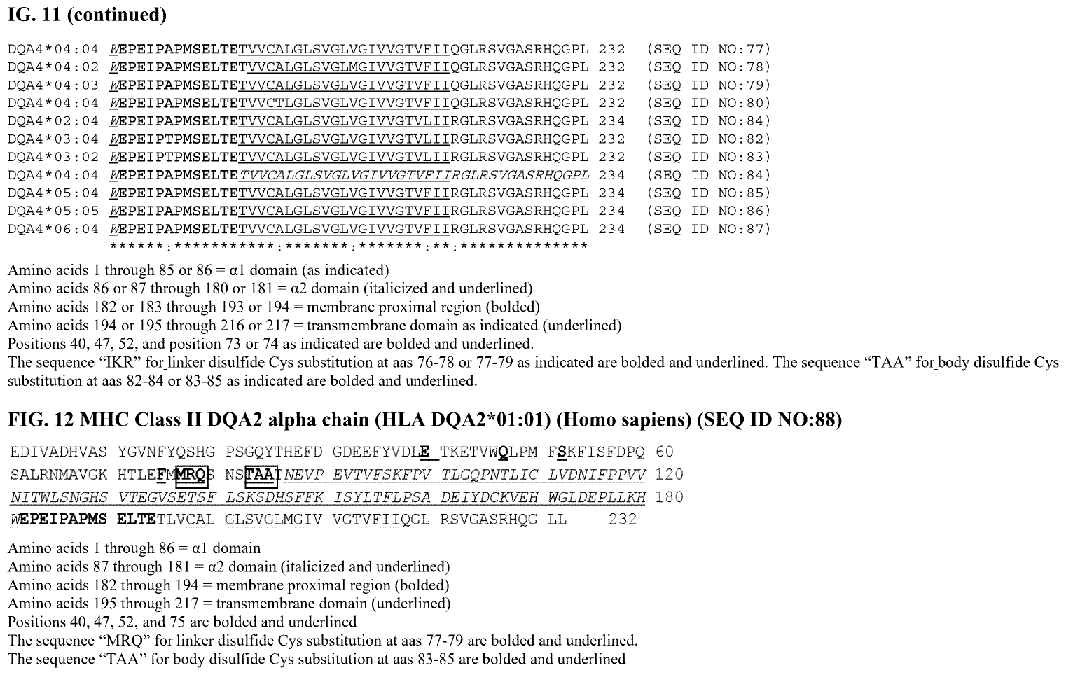

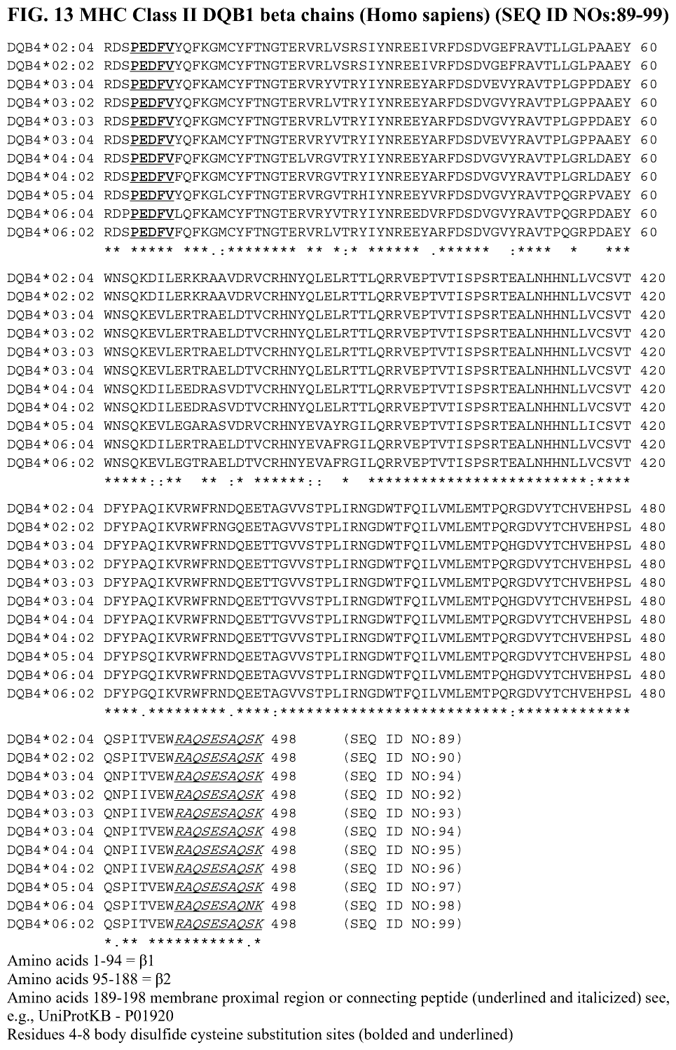

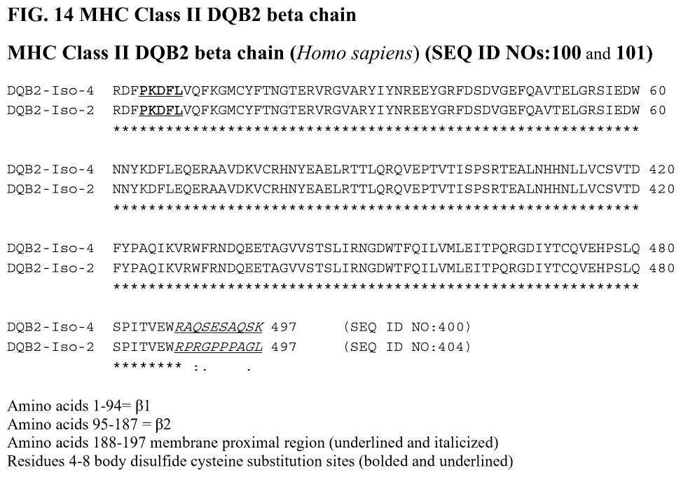

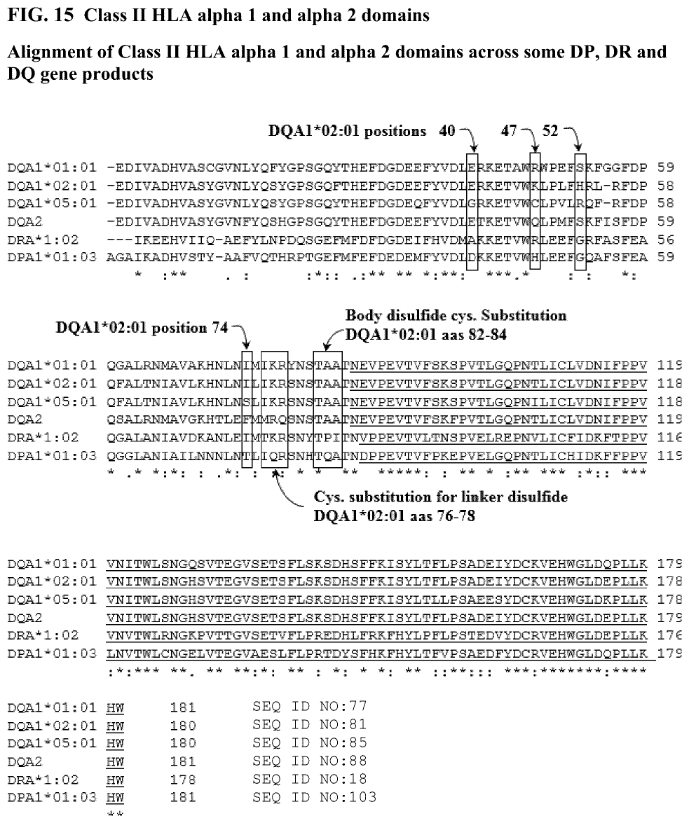

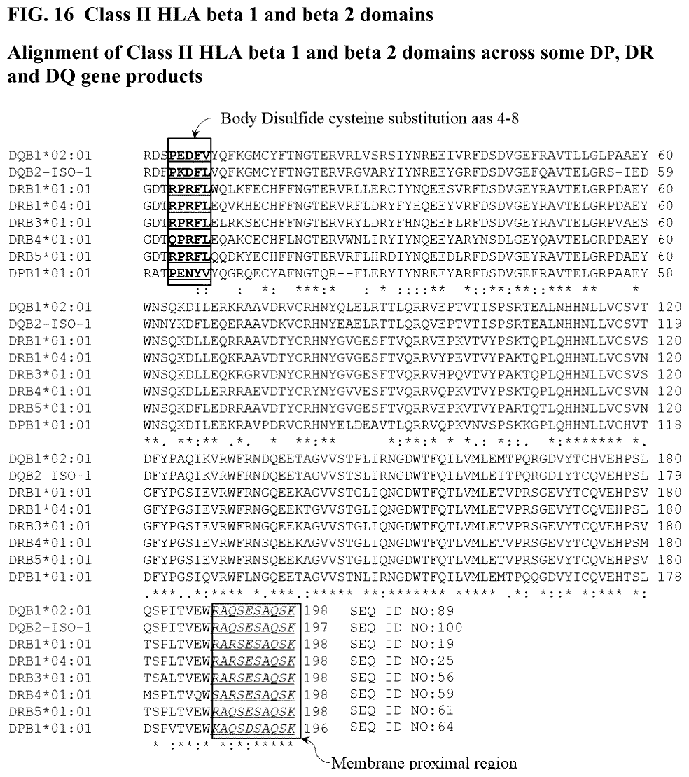

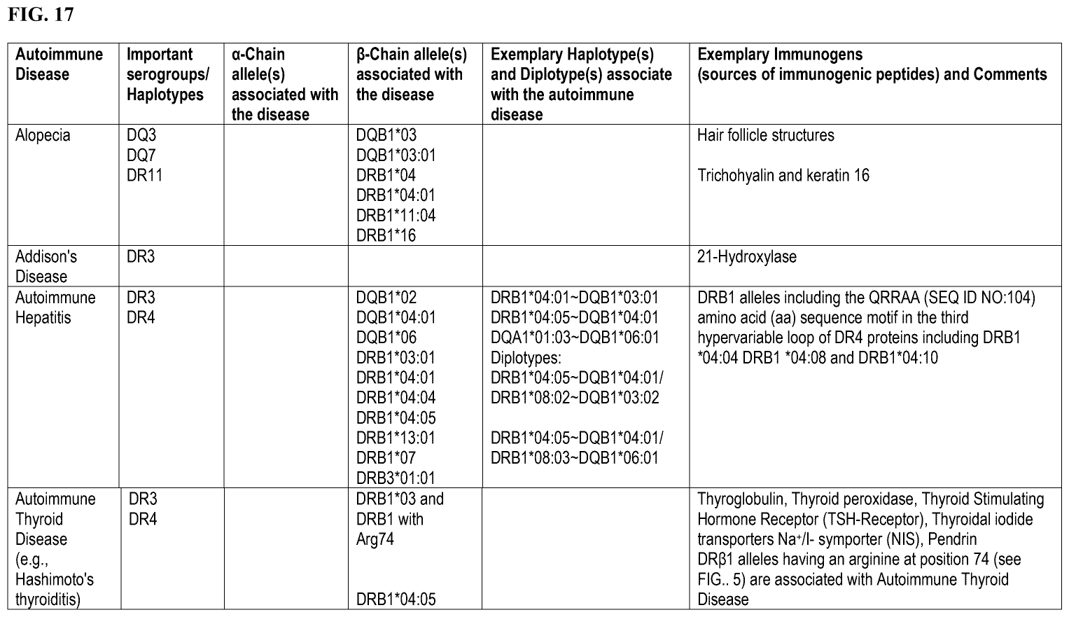

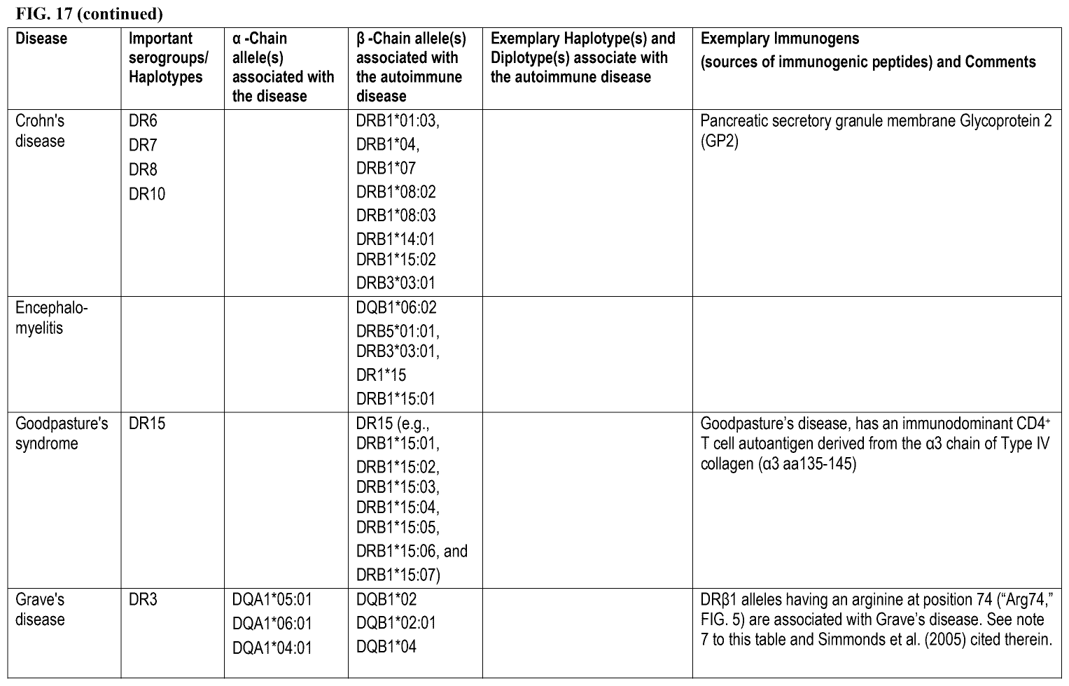

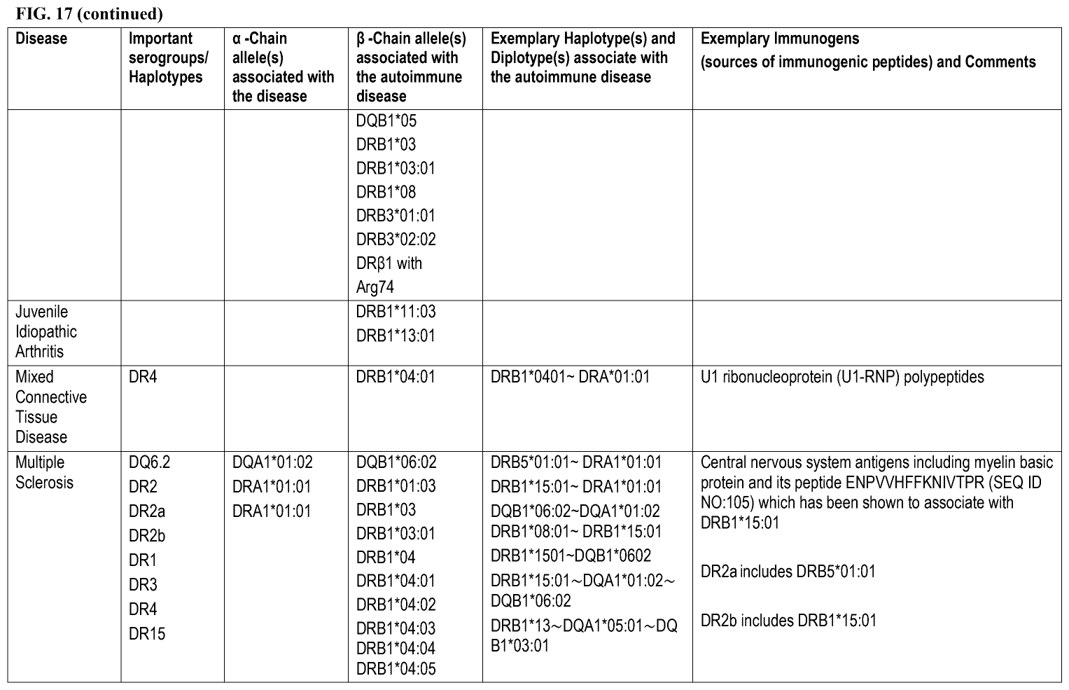

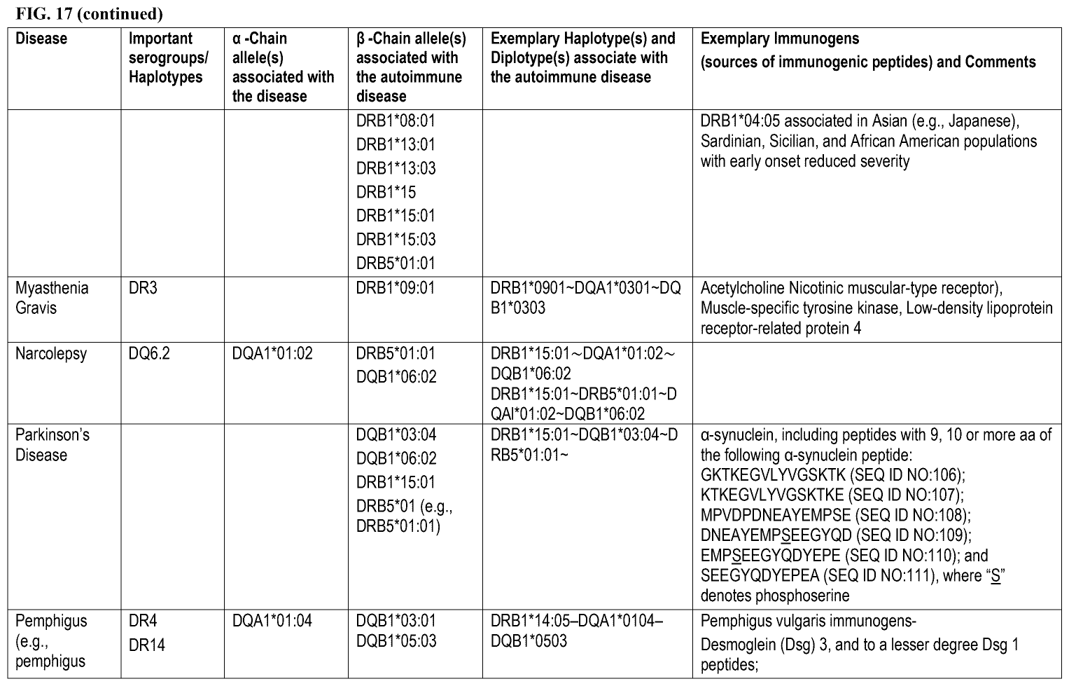

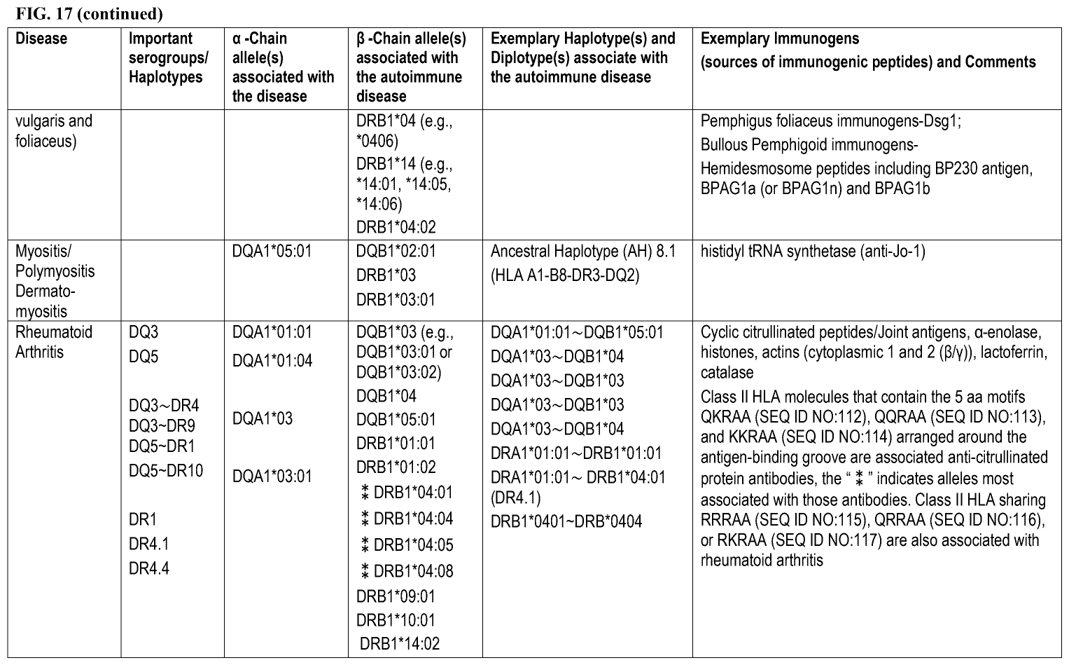

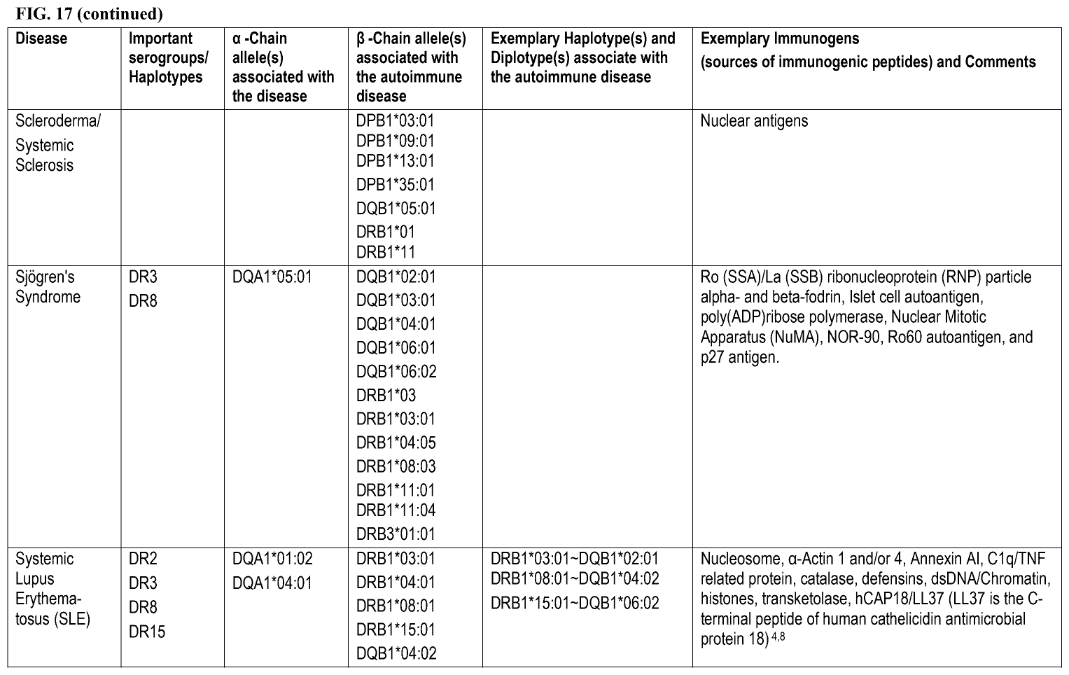

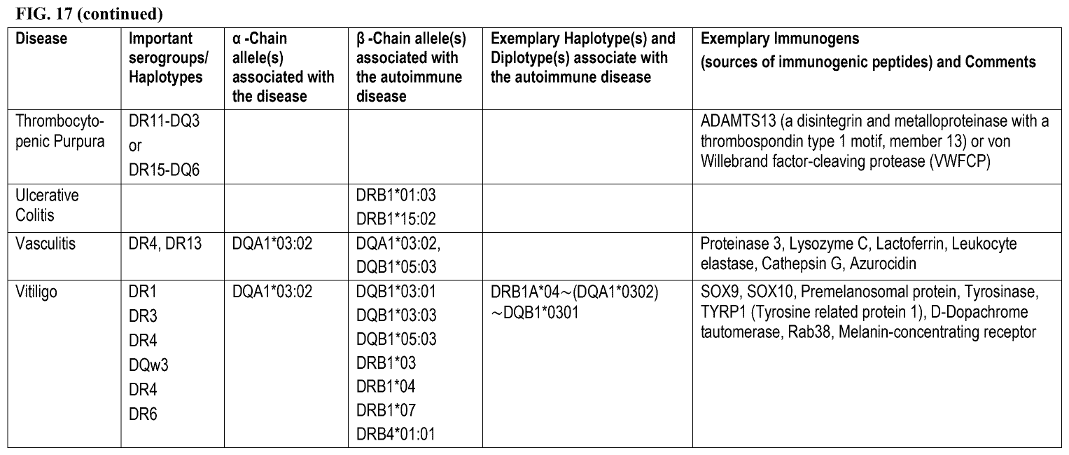

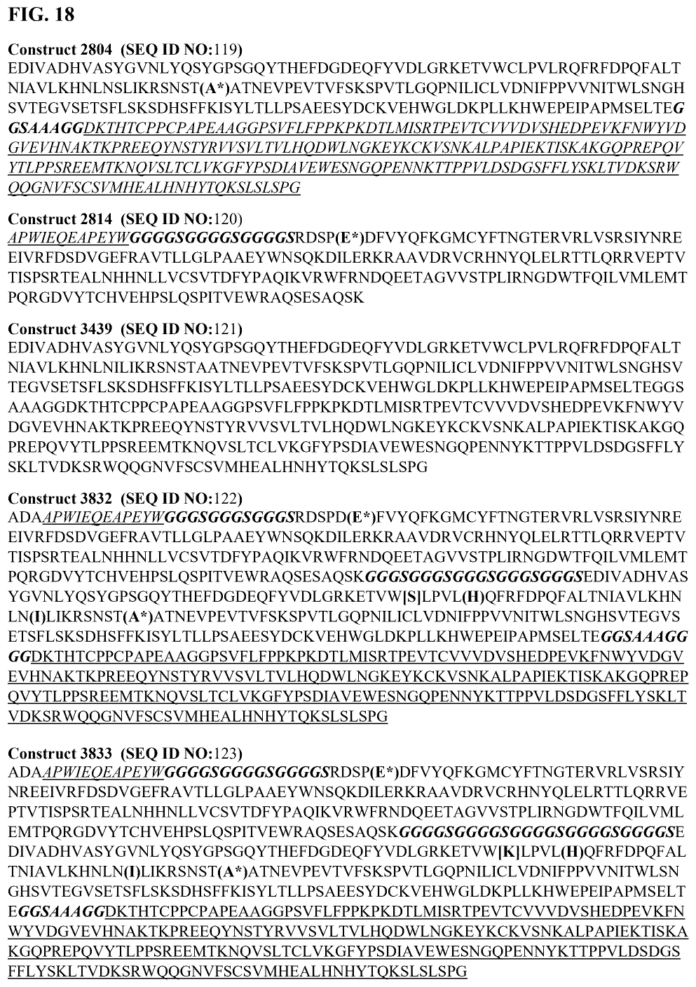

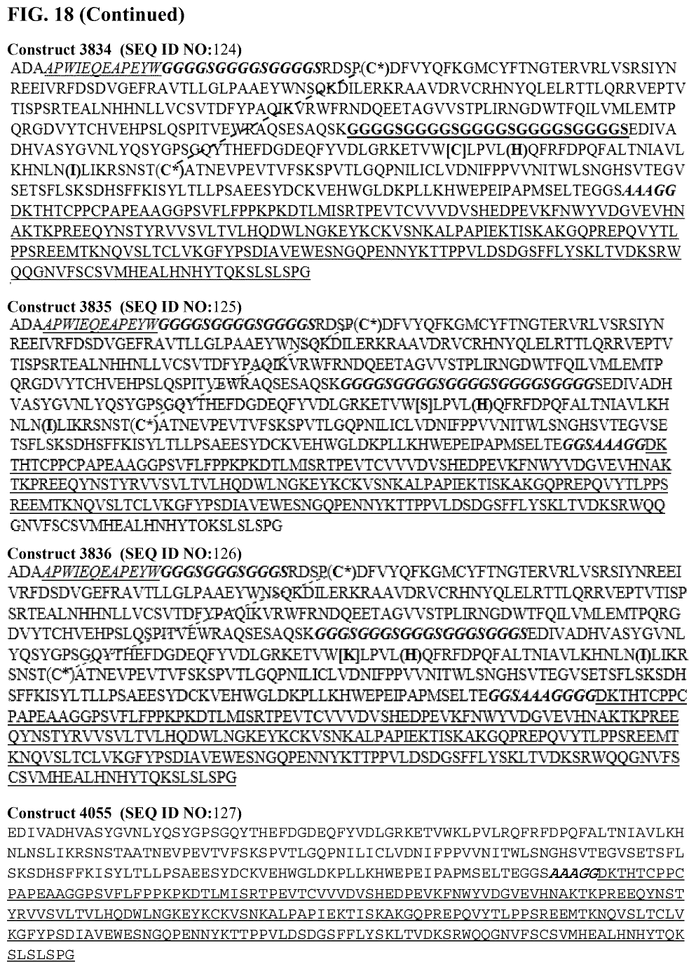

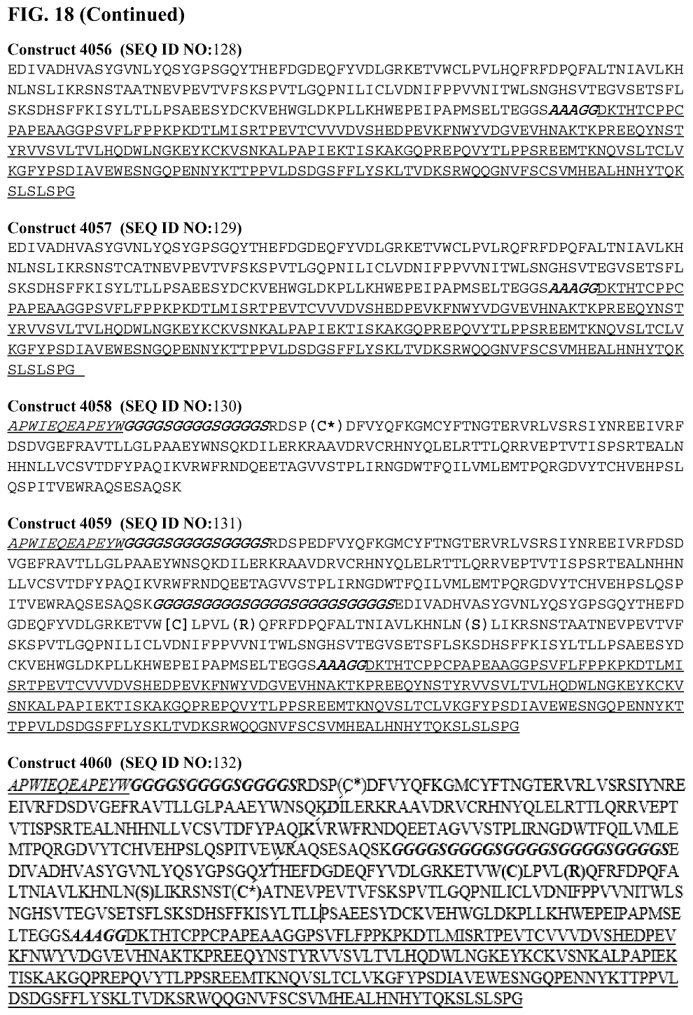

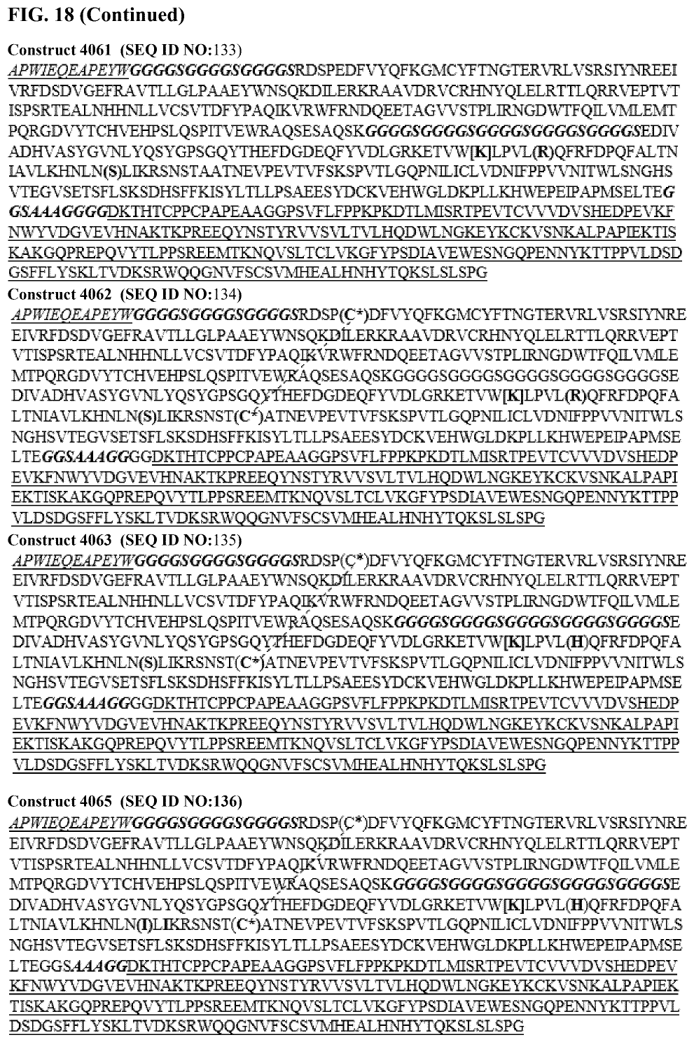

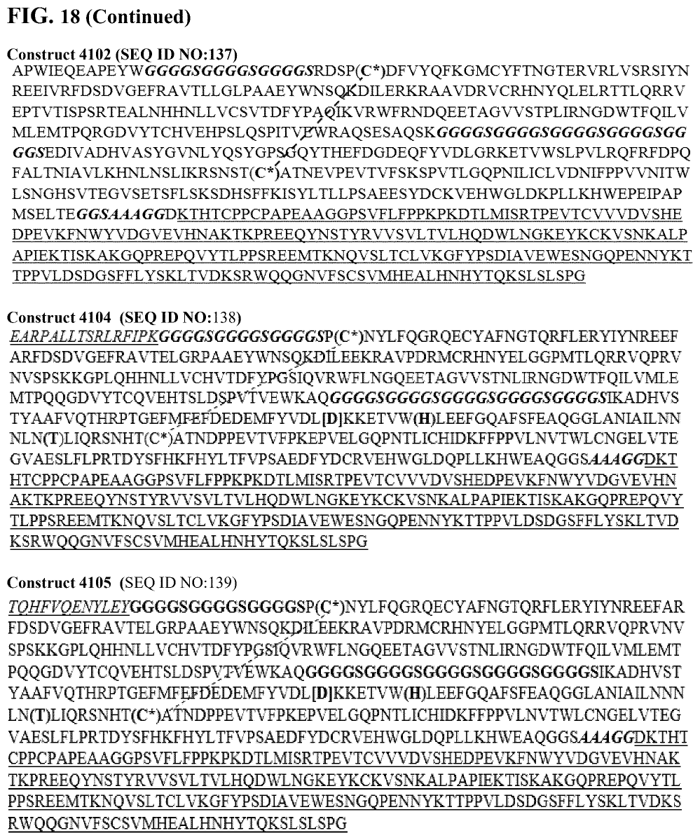

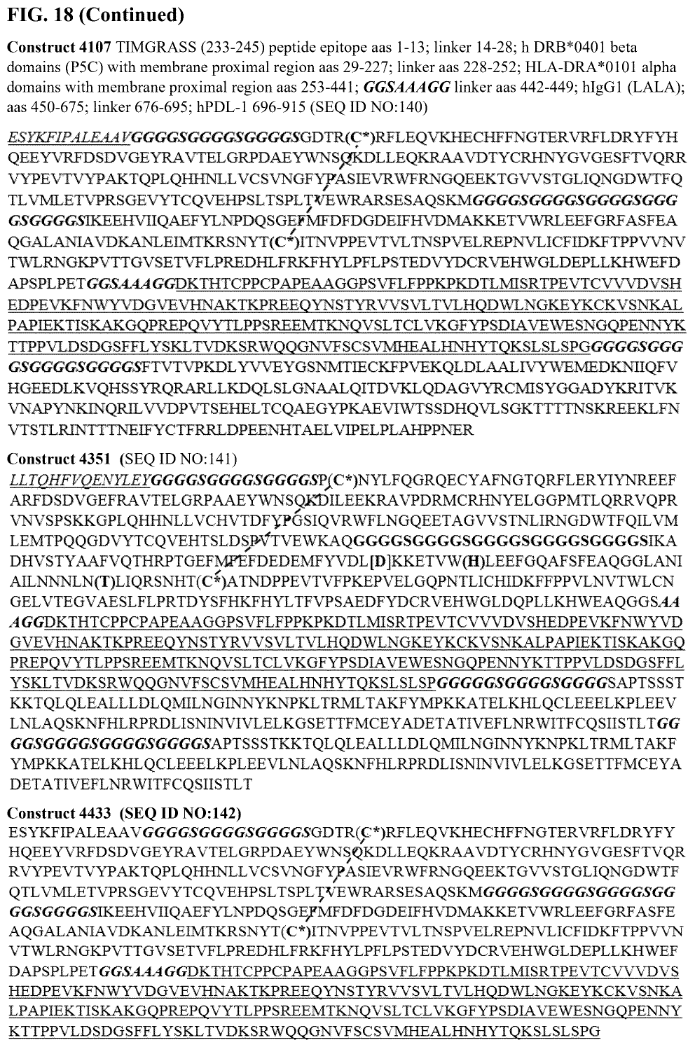

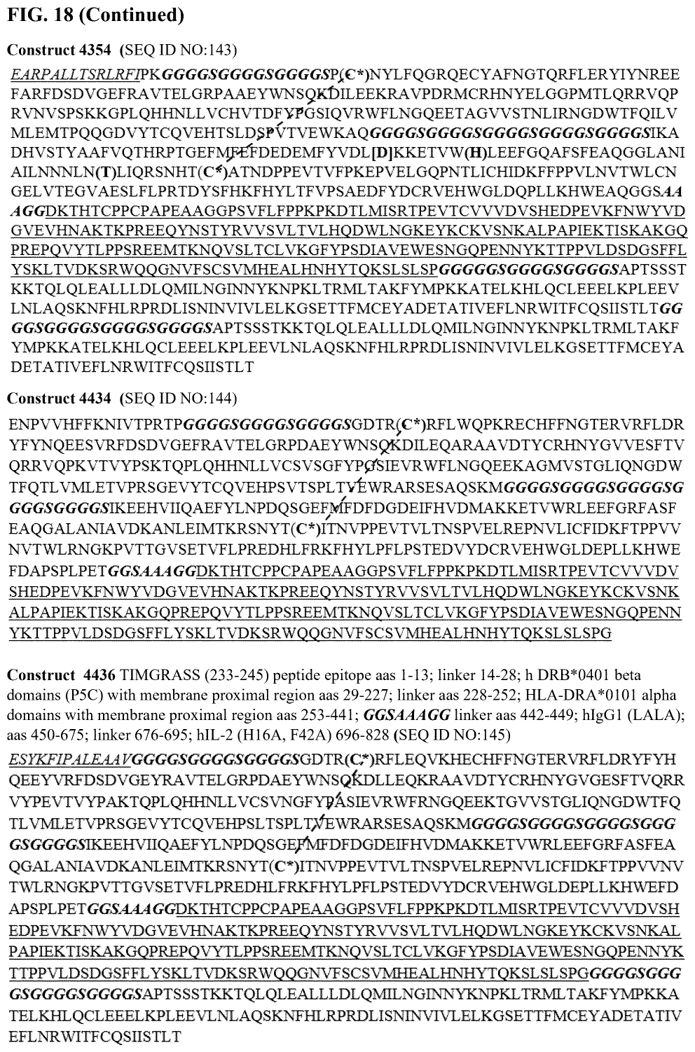

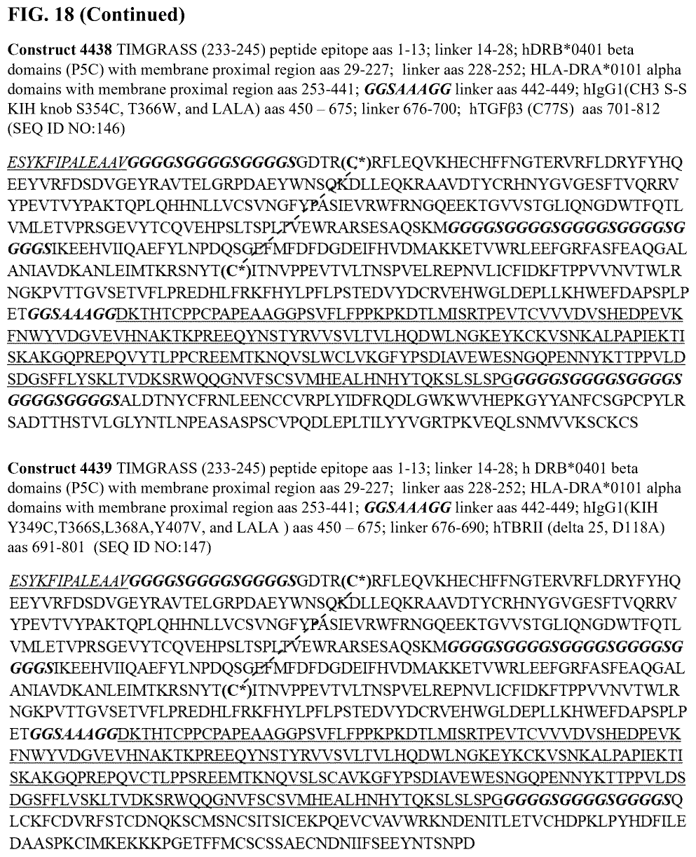

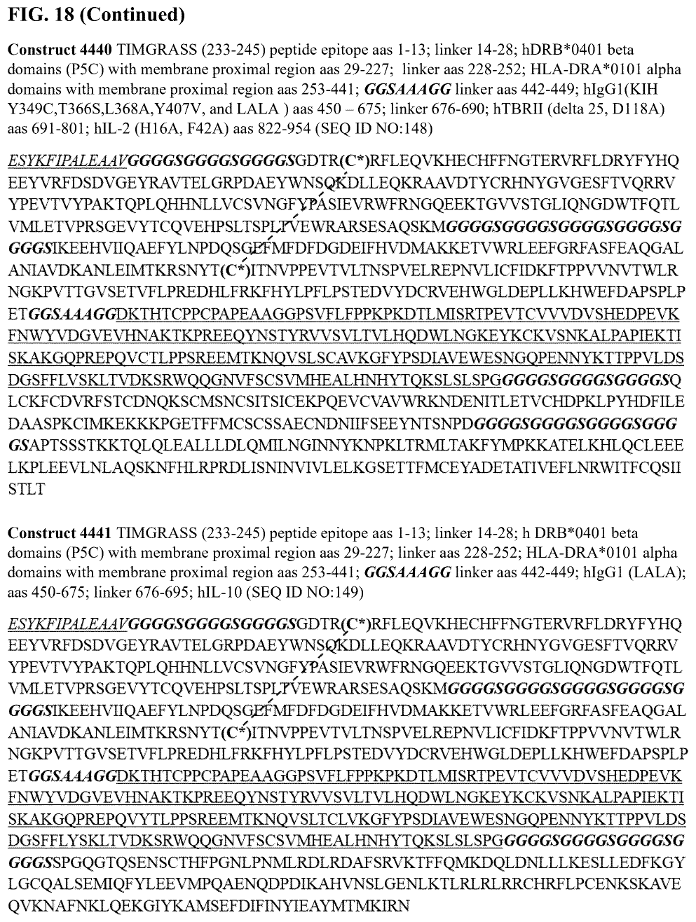

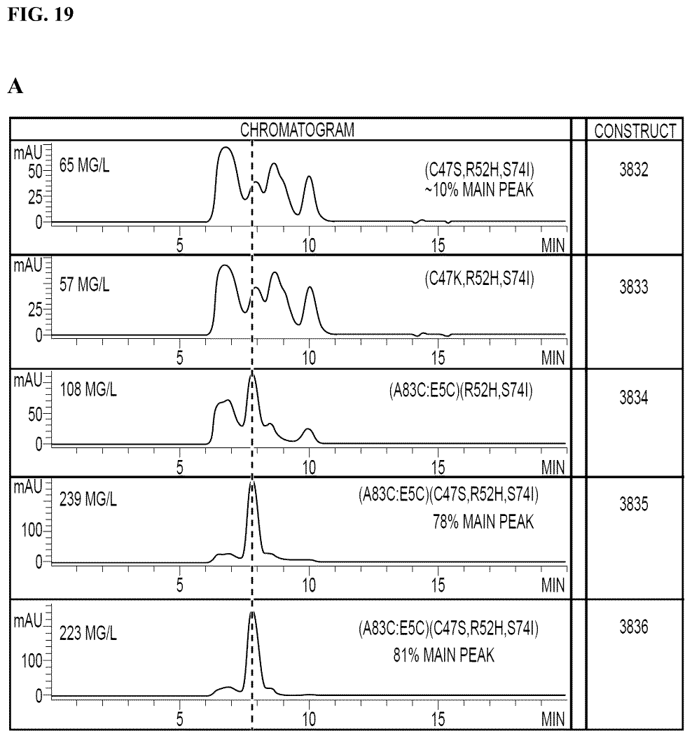

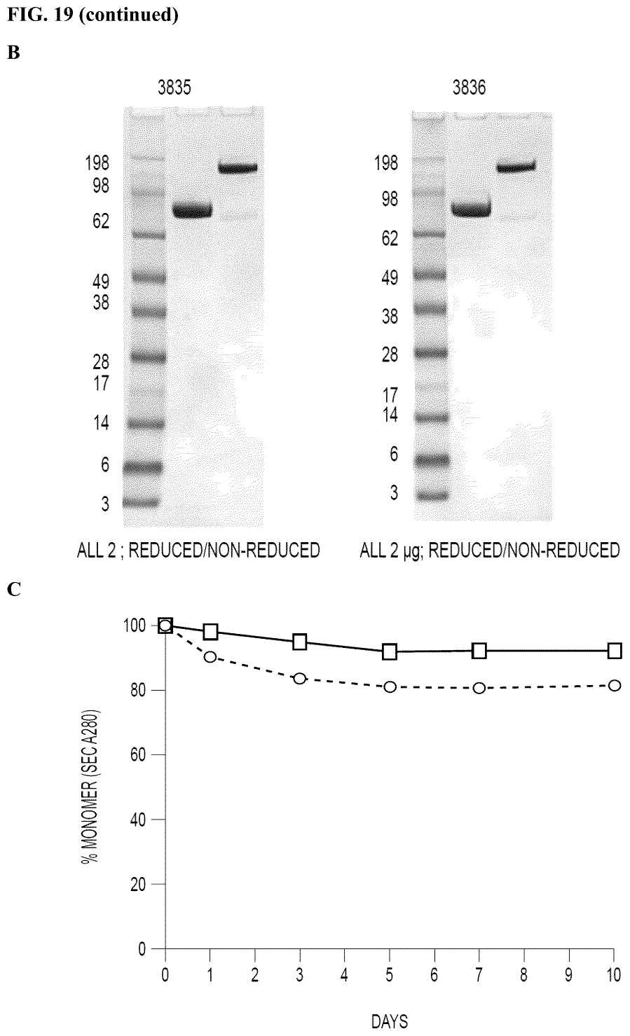

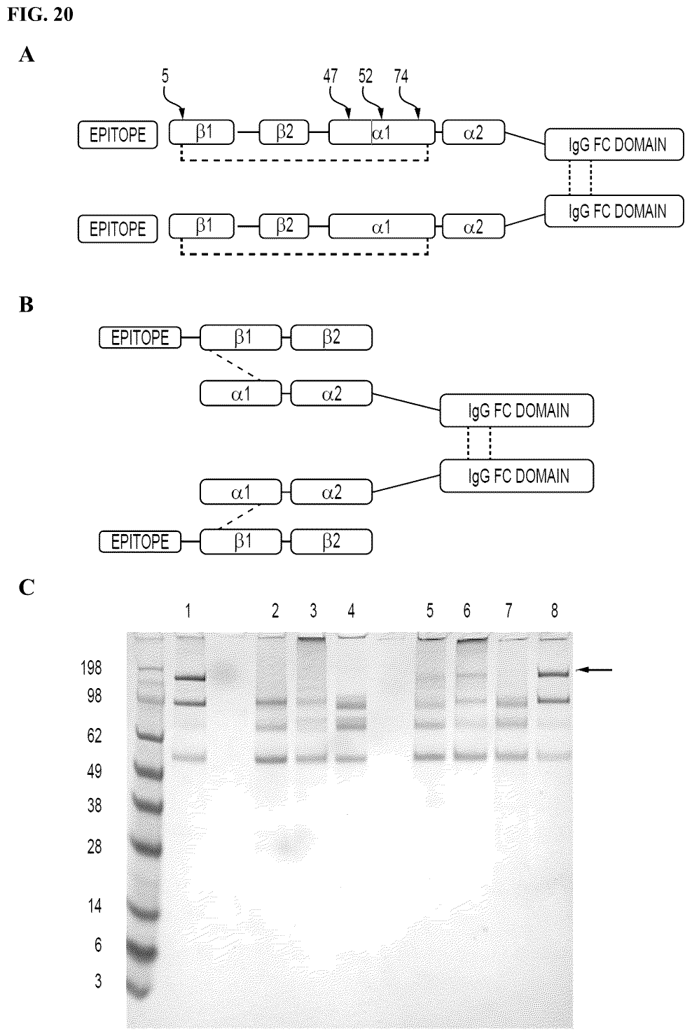

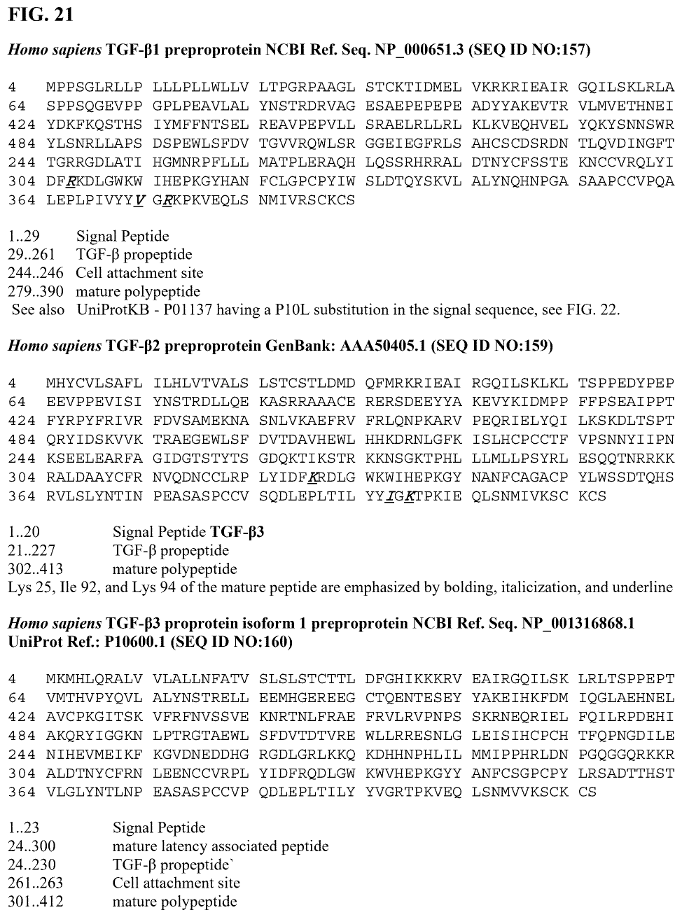

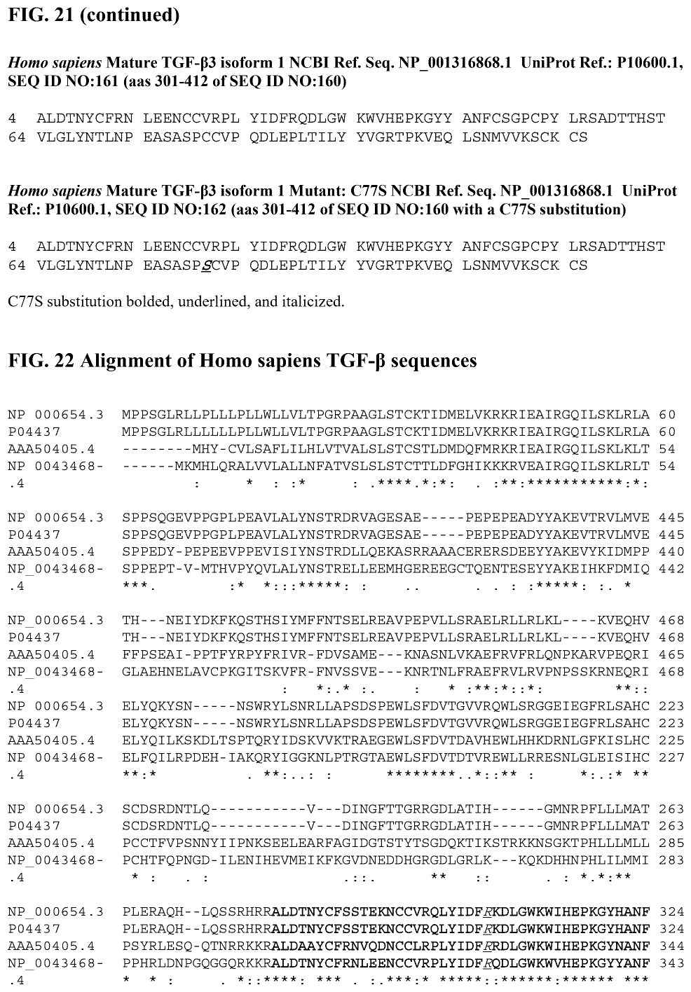

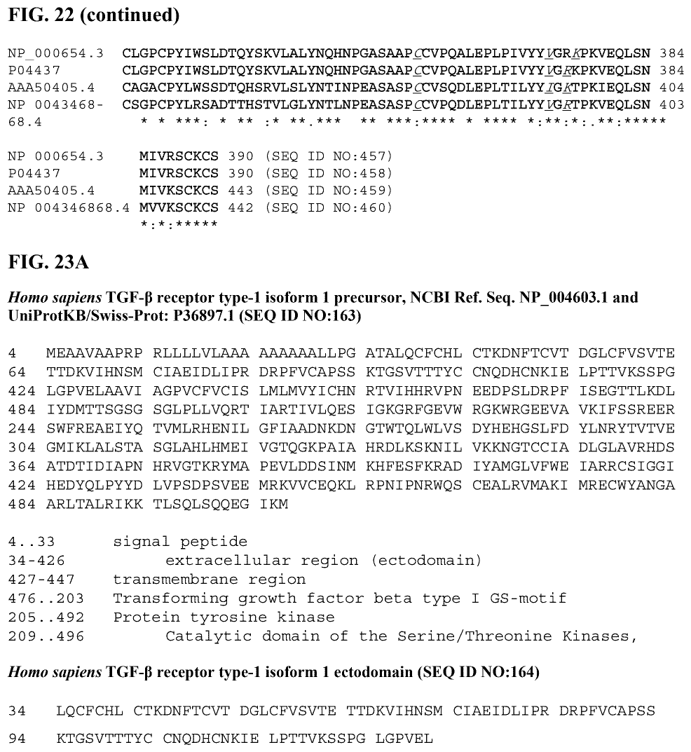

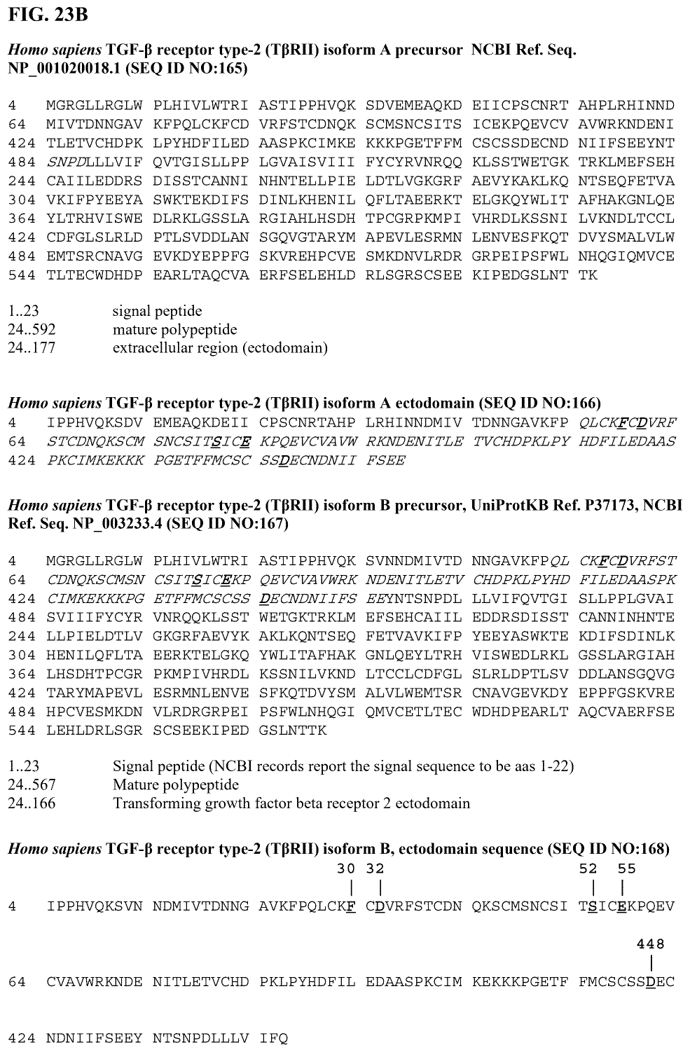

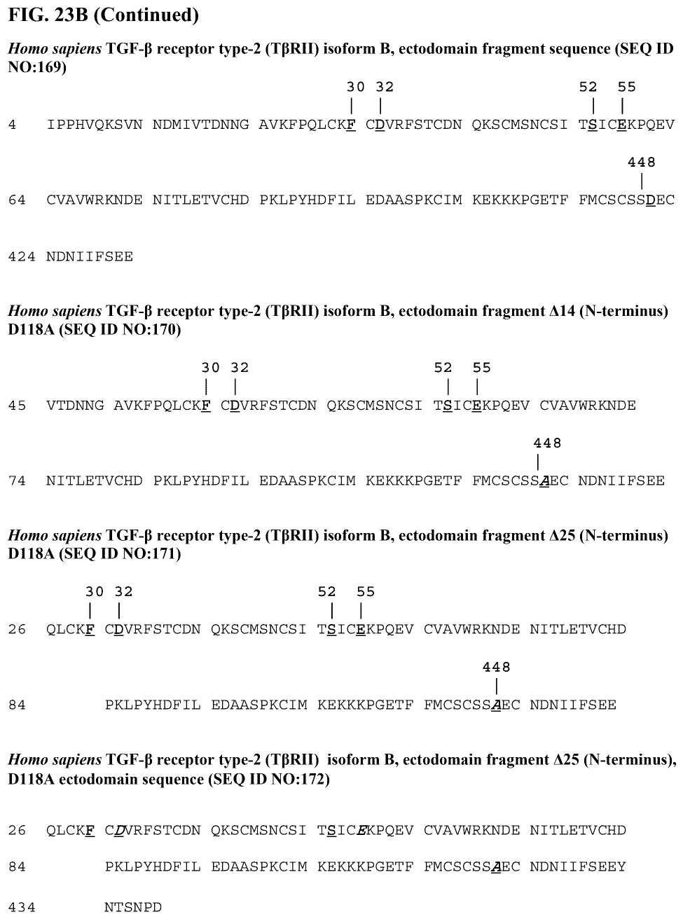

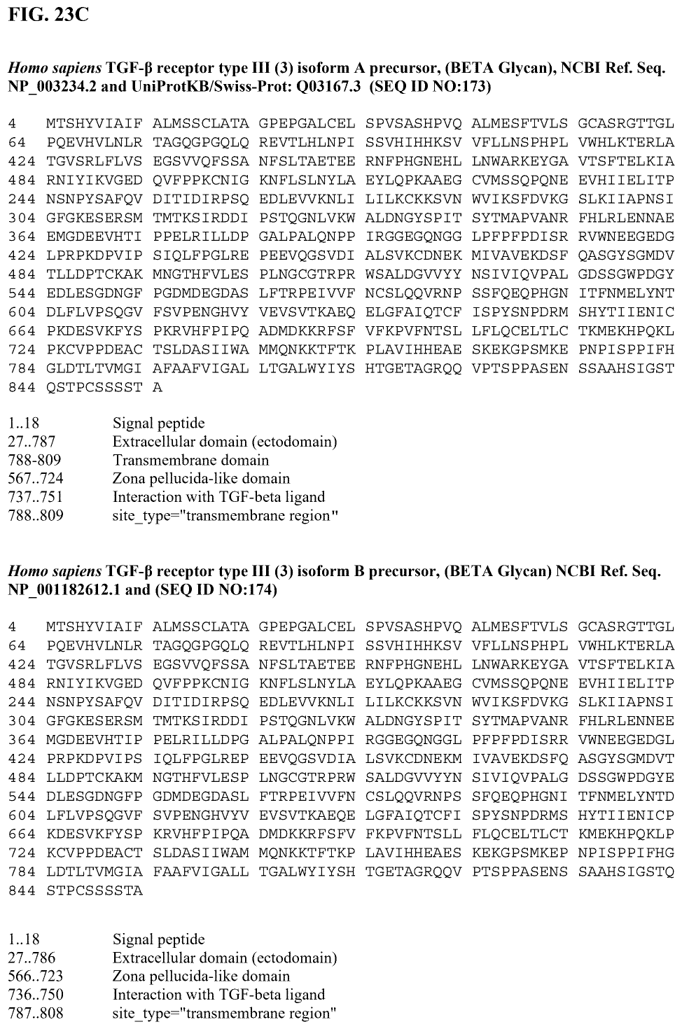

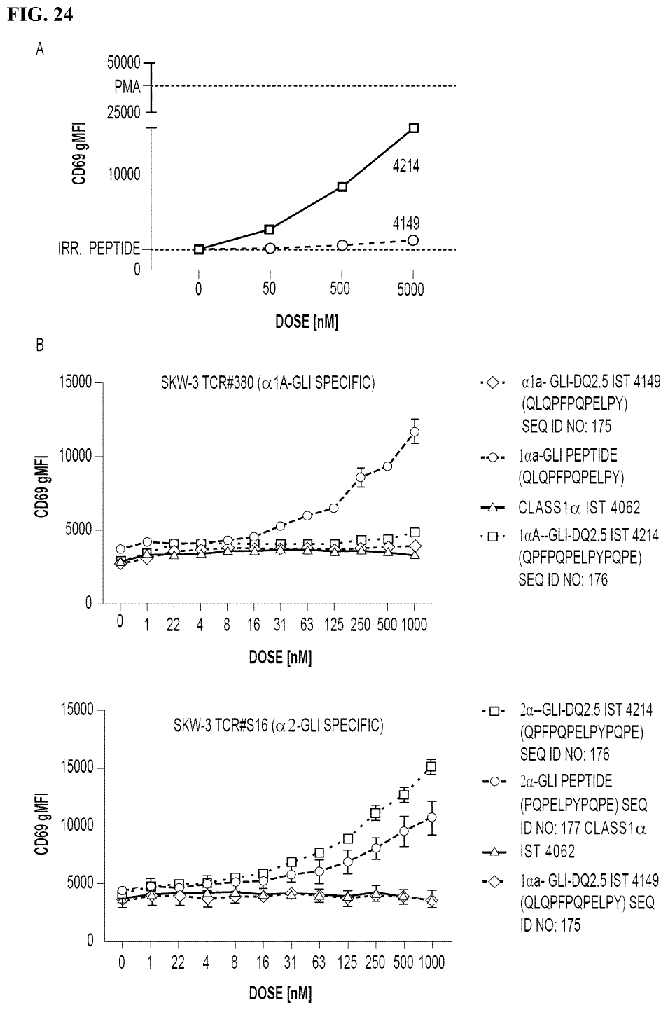

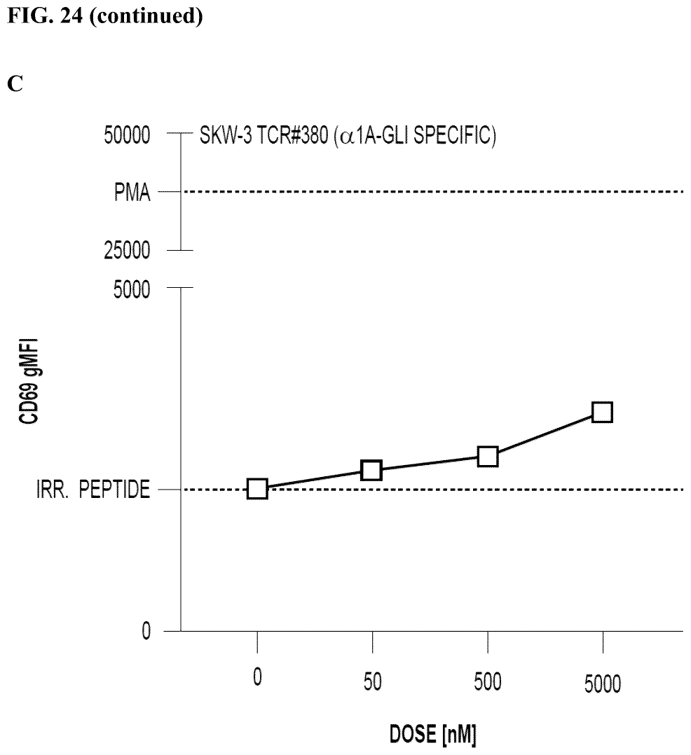

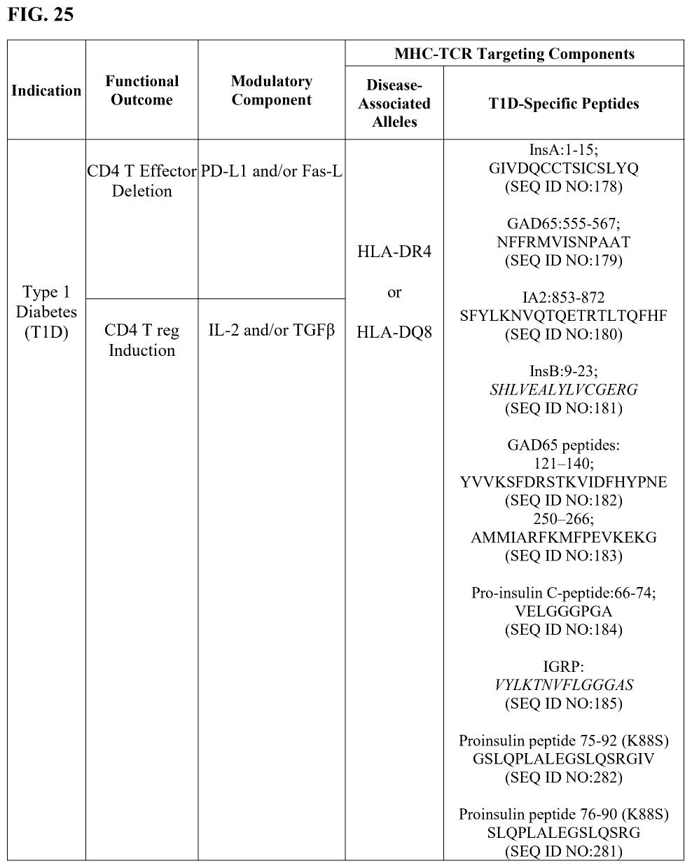

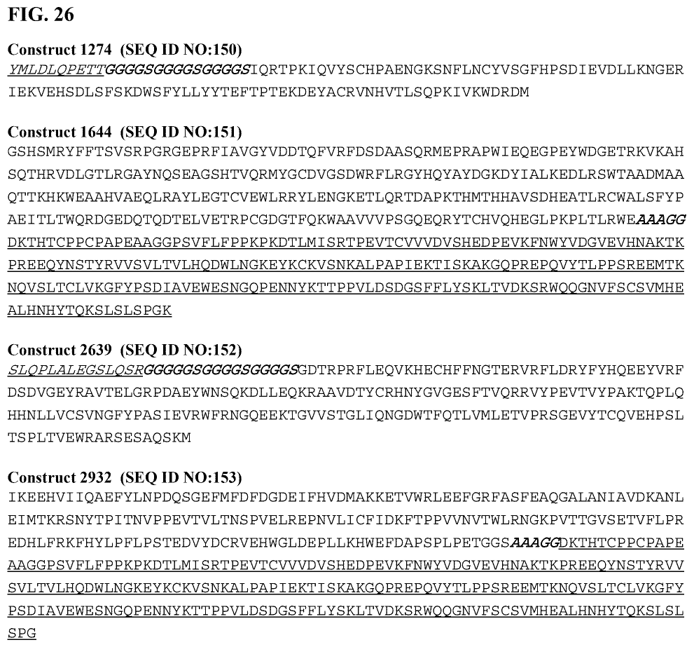

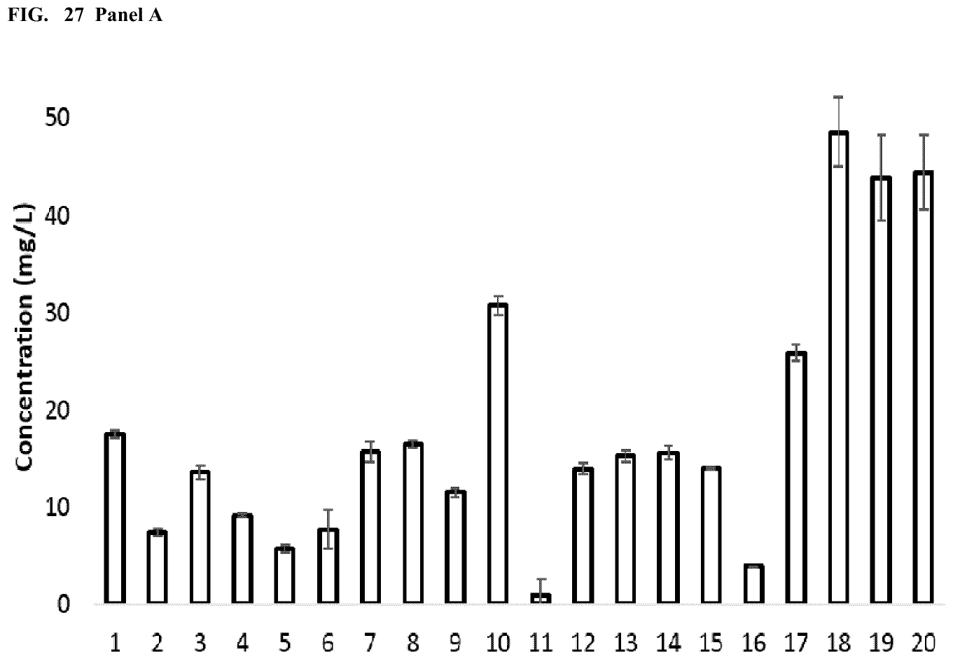

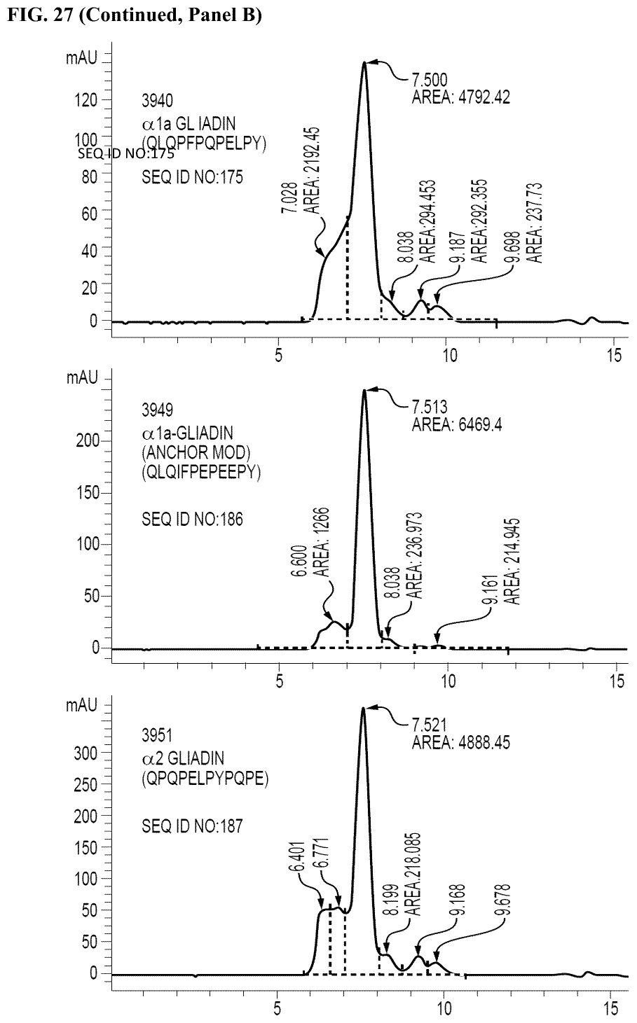

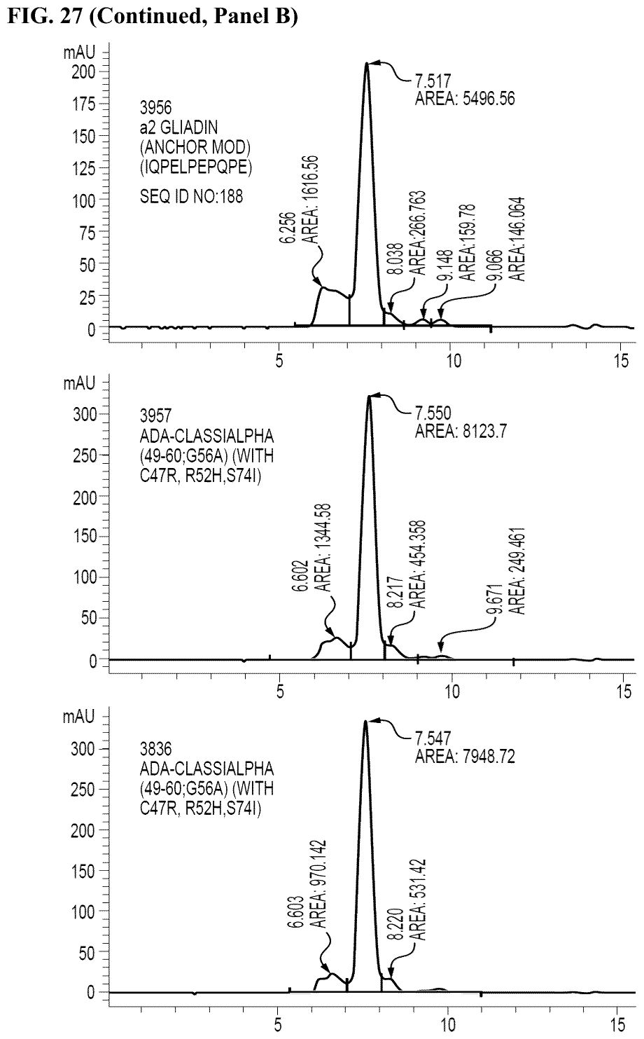

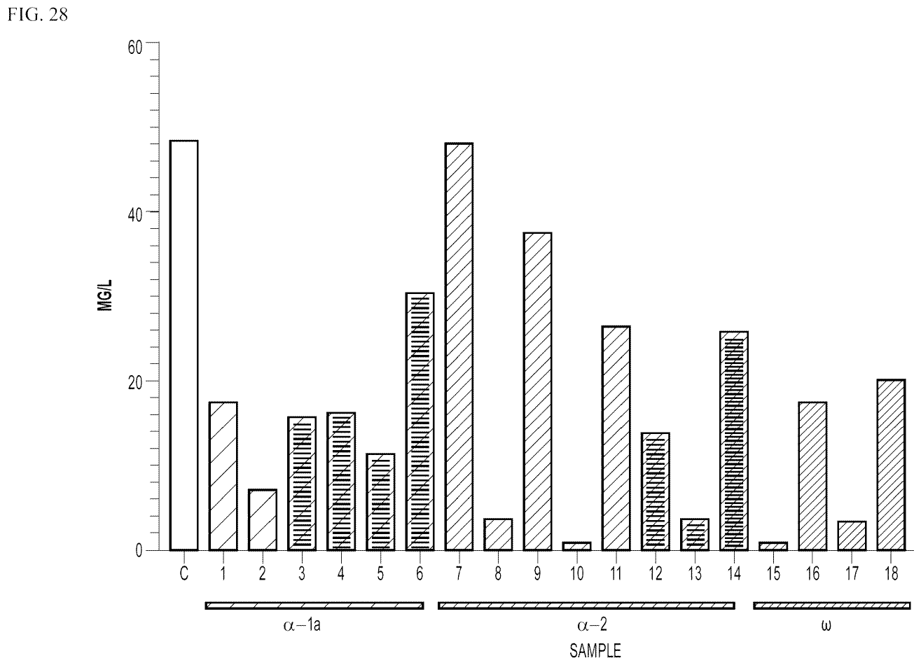

The present disclosure enables expression of MHC (HLA) Class II proteins at increased levels by introducing a combination of features that stabilize the functional heterodimer. The CIICs described herein utilize a single chain format to force α subunit (α1 and α2 domains) and β subunit (β1 and β2 domains) folding and pairing with the β subunit placed N-terminal to the α subunit. The specific ordering of the MHC domains generally is β1, β2, α1 and α2, with optional linkers located between the domains. The epitope to be bound in the CIIC binding pocket and presented to a TCR is fused to the Class II construct by a “L1” linker attached to the 131 domain of the 1 subunit's sequence. The paired a and 1 subunits are stabilized by at least one disulfide bond formed between the C-terminal portion of the α subunit's α1 domain and either the N-terminus of the 131 domain or the L1 linker attached to it (see, e.g., FIG. 1). Additional stabilization may be obtained by introducing aa substitutions that improve hydrogen bonding between the α and β subunit sequences, and/or substitutions that enhance peptide MHC (HLA) binding interactions. The increased peptide-MHC (HLA) interactions effectively increase the affinity between the peptide and the binding pocket sequences causing the peptide to have an increased residence time in the binding pocket relative to the affinity and residence time observed in the absence of the substitutions. Additionally, embodiments of the CIIC constructs described herein display resistance to denaturation (thermal stability) at elevated temperatures and upon freeze-thaw testing.

FIG. 1 shows a schematic of CIIC embodiments that can be expressed at increased levels with the N-terminus at the left. At “A” FIG. 1 shows the overall architecture of a CIIC with optional linkers Lα, Lβ and L1 through L4, along with an optional scaffold (e.g., an Ig Fc) polypeptide and/or optional additional polypeptide at the C-terminal end of the construct. The number 2 appearing above the construct represents an example of a location in the L1 linker for formation of a disulfide bond with a cysteine located in the α1 domain sequence that is described in more detail below. The other numbers appearing above the constructs are given for orientation and represent locations (e.g., aa positions) in the CIIC elements where disulfide bonds or substitutions (e.g., that benefit expression levels and/or the stability (e.g., thermal stability) and/or resistance to non-specific aggregation of the CIIC may be made. Each of thepolypeptide linkers [L1 (between the epitope and 131 domain), L2 (between the 132 and α1 domain), L3 (C-terminal and proximate to the α2 domain), Lα (between the α1 and α2 domains), Lβ (between the 131 and β2 domains), and L4 (C-terminal to the scaffold sequence, such as between the scaffold sequence and a C-terminal MOD)] may be present or absent, and are independently selectable.

Throughout the disclosure the MHC α chain (subunit) α1 and α2 domain sequences are numbered starting at 1 from the N-terminus of the α1 domain through the C-terminus of the α2 domain. The MHC β chain β1 and β2 domain sequences are numbered separately starting with the N-terminus of the 131 domain at 1 and going through to the C-terminus of the β2 domain. Linker sequences, scaffold sequences, MOD sequences, and the sequences of any additional peptides that are present in a CIIC are similarly numbered starting at their N-terminal aa. The numbering as shown in FIG. 1 in structures A, C and N is presented for exemplification and is keyed to a construct prepared from HLA DQA1*05:01 and DQB1*02:01, which make up HLA DQ 2.5. Positions 40, 47, 52, and 74 of HLA DQ 2.5, and their corresponding locations in alleles, represent locations where aa substitutions enhancing expression and stability may be made. Position 5 in the β1 domain and position 83 in the α1 domain exemplify specific aas in DQ2.5 constructs where cysteine substitutions for formation of a body disulfide bond (shown as a dashed line in A) may be made. Position 2 in the L1 linker and position 77 in the α1 domain exemplify specific aas where cysteine substitutions for formation of a linker disulfide bond (shown as a dashed line in structure E) may be made. The positions for aa substitutions and disulfide bond formation in other Class II α lleles corresponding to those in DQ2.5 may be identified by aa sequence alignment (e.g., with the Clustal Omega Version 1.2.2 available on the internet at www.ebi.ac.uk/Tools/msa/clustalo/) using FIGS. 15 and 16 to align across the α and β subunits (chains) of the individual MHC alleles of different Class II gene products (e.g., DQ, DR, and DP, gene products). Corresponding positions in some α and β subunits (chains) are provided in the aa sequence aligned in FIGS. 5 to 7, 9 to 11, and 13 to 16.

The present disclosure provides CIICs for, among other things, use in the treatment of autoimmune diseases (e.g., T1D and celiac disease) and other diseases and disorders including cancers and allergies. The elements present in the CIICs will vary depending upon whether the construct is intended to be soluble, immobilized, or membrane bound. Where the protein is intended to be soluble, or soluble and later immobilized or fused to another molecule, the MHC Class II polypeptide comprises no sequences that will cause the expressed protein to substantially associate with a cell membrane (e.g., a transmembrane domain or portion thereof). In contrast, where the protein construct is intended to be membrane associated, the constructs may include, as all or part of an additional polypeptide sequence, a sequence of aas that associates with a lipid bilayer or cell membrane (e.g., a transmembrane domain such as the transmembrane domain of the MHC Class II α subunit).

### 1. Soluble and Immobilized Class II Protein Constructs

In some instances CIICs are soluble, that is they do not comprise integral membrane protein sequences. CIICs that are to be soluble, or soluble and subsequently immobilized or fused to another molecule, do not comprise aa sequences that directly associate with the hydrophobic portion of a cell membrane (e.g., a transmembrane MAS, amphipathic helix, lipid, or substantial portion thereof). Similarly, they do not comprise polypeptide sequences that result in the addition of hydrophobic groups (e.g., hydrocarbon groups or moieties as lipid additions, such as prenylation sequences or sequences that result in the addition of glycosylphosphatidylinositol anchors) that would cause the CIIC to associate substantially or completely with a cell membrane or other lipid bilayer. Soluble CIICs can become peripherally associated with membranes through interactions with polar head groups of membrane lipids, surface carbohydrates etc.; however, for the purpose of this disclosure peripherally associated CIICs are still considered a form of soluble protein. Accordingly, otherwise soluble CIICs may become bound to lipid bilayers or cell membranes as peripheral membrane proteins where the bilayers or membranes contain (e.g., cells express) surface proteins or other molecules which can interact with any portion of a CIIC, including but not limited to a portion of a scaffold (e.g., an Ig Fc) or an additional polypeptide.

In various embodiments, soluble CIICs comprise as a single amino acid sequence the polypeptide components: a peptide epitope, an optional linker (L1), an MHC Class II β chain (subunit) sequence comprising β1 and β2 domain sequences (optionally including membrane proximal sequences), an MHC Class II α chain (subunit) sequence comprising the α1 and α2 domain sequences (optionally including membrane proximal sequences), and optionally an additional polypeptide sequence. The polypeptide components of the constructs may appear from N-terminus to C-terminus in that order, and may comprise one or more additional linker sequences that are selected independently between the components, one or more stabilizing disulfide bonds, and/or amino acid substitutions (e.g., for stabilizing the CIIC). MOD-containing CIICs further comprise at least one (e.g., at least two) wild-type or variant MOD sequences located C-terminal to the α2 domain sequence. The MOD(s) may be attached to the α2 domain sequence itself, or to an element selected from a membrane proximal sequence, a scaffold, or an additional polypeptide sequence that is attached directly or indirectly to the C-terminus of the α2 domain sequence. The MOD(s) may be attached to any of those sequences via a linker, and independently selected linker peptide sequences may be located between any of those CIIC elements.

Accordingly, a first CIIC embodiment comprises as a single aa sequence (e.g., from N-terminus to C-terminus): (i) a peptide epitope aa sequence; (ii) optionally an L1 aa linker sequence; (iii) an MHC Class II β chain (subunit) polypeptide sequence (comprising e.g., the β1 and β2 domain sequences); (iv) an optional L2 aa linker sequence; (v) an MHC Class II α chain (subunit) polypeptide sequence (comprising e.g., the α1 and α2 domain sequences); (vi) an optional L3 aa linker sequence; (vii) optionally a scaffold sequence and/or MAS; (viii) optionally an L4 linker; and (ix) optionally one or more (e.g., two or more) MOD and/or additional polypeptide sequences; wherein the Class II polypeptide optionally comprises a disulfide bond between the β1 domain (e.g., from a cysteine substituted for one of the N-terminal 8 aas) and the α1 domain (e.g., a cysteine substituted for one of the C-terminal 11 aas of the α1 domain) and/or a disulfide bond between a cysteine in an L1 linker present in the CIIC and a cysteine in the α1 domain (e.g., a cysteine substituted for one of the C-terminal 11 aas of the α1 domain); and wherein, when the Class II polypeptide comprises a cysteine at aa 43 through aa 48 of the α1 domain sequence, it is optionally substituted by an aa other than cysteine (e.g., a S, R or K such as a C47S, C47R or C47K substitution in DQA*05:01).

A second CIIC embodiment comprises as a single aa sequence from N-terminus to C-terminus: (i) a peptide epitope aa sequence; (ii) optionally an L1 aa linker sequence; (iii) an MHC Class II β chain (subunit) polypeptide sequence comprising a 11 and β2 domain sequence; (iv) an optional L2 aa linker sequence; (v) an MHC Class II α chain (subunit) polypeptide sequence comprising an α1 and α2 domain sequence; (vi) an optional L3 aa linker sequence; (vii) optionally a scaffold sequence and/or MAS; (viii) optionally an L4 linker; and (ix) optionally one or more (e.g., two or more) MOD and/or additional polypeptide sequences; wherein the Class II polypeptide optionally comprises a disulfide bond between the β1 domain (e.g., from a cysteine substituted for one of the N-terminal 8 aas) and the α1 domain (e.g., a cysteine substituted for one of the C-terminal 11 aas of the α1 domain) and/or a disulfide bond between a cysteine in an L1 linker present in the CIIC and a cysteine in the α1 domain (e.g., a cysteine substituted for one of the C-terminal 11 aas of the α1 domain); and wherein, when the Class II polypeptide comprises a cysteine at aa 43 through aa 48 of the α1 domain sequence, it is optionally substituted by an aa other than cysteine (e.g., a S, R or K).

A third CIIC embodiment comprises as a single aa sequence from N-terminus to C-terminus: (i) a peptide epitope aa sequence; (ii) a L1 aa linker sequence; (iii) an MHC Class II β chain (subunit) polypeptide sequence comprising a 11 and β2 domain sequence; (iv) an optional L2 aa linker sequence; (v) an MHC Class II α chain (subunit) polypeptide sequence comprising an α1 and α2 domain sequence; (vi) an optional L3 aa linker sequence; (vii) a scaffold sequence and/or MAS; (viii) optionally an L4 linker; and (ix) optionally one or more (e.g., two or more) MOD and/or additional polypeptide sequences; wherein the Class II polypeptide optionally comprises a disulfide bond between the β1 domain (e.g., from a cysteine substituted for one of the N-terminal 8 aas) and the α1 domain (e.g., a cysteine substituted for one of the C-terminal 11 aas of the α1 domain) and/or a disulfide bond between a cysteine in an L1 linker present in the CIIC and a cysteine in the α1 domain (e.g., a cysteine substituted for one of the C-terminal 11 aas of the α1 domain); and wherein, when the Class II polypeptide comprises a cysteine at aa 43 through aa 48 of the α subunit aa sequence (i.e., at aa positions 43-48 of the α1 portion of the α1 and α2 domain sequences), it is optionally substituted by an aa other than cysteine (e.g., a S, R or K).

A fourth CIIC embodiment comprises as a single aa sequence from N-terminus to C-terminus: (i) a peptide epitope aa sequence; (ii) optionally an L1 aa linker sequence; (iii) an MHC Class II β chain (subunit) polypeptide sequence comprising a 11 and β2 domain sequence; (iv) an L2 aa linker sequence; (v) an MHC Class II α chain (subunit) polypeptide sequence comprising an α1 and α2 domain sequence; (vi) an L3 aa linker sequence; (vii) a scaffold sequence and/or MAS; (viii) an L4 linker; and (ix) optionally one or more (e.g., two or more) MOD and/or additional polypeptide sequences; wherein the Class II polypeptide optionally comprises a disulfide bond between the β1 domain (e.g., from a cysteine substituted for one of the N-terminal 8 aas) and the α1 domain (e.g., a cysteine substituted for one of the C-terminal 11 aas of the α1 domain) and/or a disulfide bond between a cysteine in an L1 linker present in the CIIC and a cysteine in the α1 domain (e.g., a cysteine substituted for one of the C-terminal 11 aas of the α1 domain); and wherein, when the Class II polypeptide comprises a cysteine at aa 43 through aa 48 of the α aa sequence (i.e., at aa positions 43-48 of the α1 portion of the α1 and α2 domain sequences), it is optionally substituted by an aa other than cysteine (e.g., a S, R or K).

A fifth CIIC embodiment comprises as a single aa sequence from N-terminus to C-terminus: (i) a peptide epitope aa sequence; (ii) an L1 aa linker sequence; (iii) an MHC Class II β chain (subunit) polypeptide sequence comprising a 11 and β2 domain sequence; (iv) an optional L2 aa linker sequence; (v) an MHC Class II α chain (subunit) polypeptide sequence comprising an α1 and α2 domain sequence; (vi) an optional L3 aa linker sequence; (vii) a scaffold sequence; (viii) optionally an L4 linker; and (ix) one or more (e.g., two or more) MOD and/or additional polypeptide sequences; wherein the Class II polypeptide optionally comprises a disulfide bond between the β1 domain (e.g., from a cysteine substituted for one of the N-terminal 8 aas) and the α1 domain (e.g., a cysteine substituted for one of the C-terminal 11 aas of the α1 domain) and/or a disulfide bond between a cysteine in an L1 linker present in the CIIC and a cysteine in the α1 domain (e.g., a cysteine substituted for one of the C-terminal 11 aas of the α1 domain); and wherein when the Class II polypeptide comprises a cysteine at aa 43 through aa 48 of the α subunit α1 and α2 domain sequence, it is optionally substituted by an aa other than cysteine (e.g., a S, R or K).

In any of the first through fifth CIIC embodiments, either or both of the MHC Class II α chain or the MHC Class II β chain polypeptide sequence may comprise an independently selected membrane proximal sequence (e.g., the α2 domain and/or the β2 domain sequence is followed at their C-terminus by an independently selected membrane proximal sequence). In any of the first through fifth CIIC embodiments, including those where, e.g., the α2 domain and/or the β2 domain sequence is followed at their C-terminus by an independently selected membrane proximal sequence, the CIIC may comprise an immunoglobulin sequence (e.g, an Ig Fc) as the scaffold sequence.

Where the first through fifth CIIC embodiments comprise a scaffold polypeptide sequence, the scaffold may stabilize (e.g., increase its thermal stability and/or prevent nonspecific aggregation) the CIIC or confer other properties to the CIIC (see, e.g., FIG. 1, structures A-I and M-P). In one instance the scaffold polypeptide comprises the sequence of an immunoglobulin (e.g., a CH2 and/or CH3 domain, or an Ig Fc polypeptide), which, in addition to potentially increasing the circulation half-life in vivo (in blood), can dimerize forming a duplex of the CIICs (see, e.g., FIG. 1, structures I and P). Sequences giving rise to ADCC and/or CDC may also be present or absent from the Ig Fc polypeptide sequences incorporated into the CIICs described herein. When the ADCC and/or CDC sequences are present the CIIC may be used to deplete the population of T cells that recognize the epitope presented by the construct. Scaffold polypeptides also include other sequences that can self-assemble to form higher order constructs, such as polypeptides comprising leucine zipper domains. Scaffold polypeptides also include other proteins such as human serum albumin and the like, in which case the construct may be considered a fusion protein.

In CIICs (e.g., any of the first through fifth CIIC embodiments) the L1 through L4 linkers may each optionally be present and selected independently. Although typically not present, linkers between the MHC Class II α chain polypeptide α1 and α2 domain sequences (“La” see FIG. 1) and the MHC Class II β chain polypeptide β1 and β2 domain sequences (“Lp” see FIG. 1) may be present. Where either or both of Lα and Lβ are present, they may be selected independently.

Any of the first through fifth CIIC embodiments may include one or more additional polypeptides that, among other things, may stabilize CIICs, provide a labeling sequence for detection, provide a sequence to be used in purification of CIICs, and/or confer other properties to the construct. Additional polypeptides may be located between any of the components, or as part of linker sequences particularly if short (e.g., 12 aas or less or 8 aas or less, such as a FLAG or 6× His tag); however, they will typically be located to the C-terminal side of the α2 domain sequence. Where a scaffold sequence is present the additional polypeptide sequence may be located N-terminal to the scaffold (e.g., between the α2 domain sequence and the scaffold, see FIG. 1, structure A), incorporated within the scaffold (particularly if short such as 12 aas or less or 8 aas or less), or C-terminal to the scaffold.

Where the additional polypeptides are affinity sequences (e.g., FLAG tags or 6× His) or antigenic determinates, the sequences permit the otherwise soluble CIICs to be immobilized. In some instances, the construct may be immobilized using one or more antibodies that recognize the affinity sequence (or another part of the CIIC). In other instances, it is possible to immobilize the CIICs on matrices that interact with the affinity sequences (e.g., resins or matrices with nickel or cobalt and 6× His domain affinity sequences). Accordingly, the CIICs may be immobilized upon sensor surfaces or other solid or semi-solid (e.g., gel) matrices bearing counterpart to the additional polypeptide. Immobilization may be used as part of a purification process. Additional polypeptide sequences also include targeting sequences (e.g., scFv or nanobody sequences) that can bind to specific components of, for example, a cell or tissue, and localize the CIICs in vivo or in vitro.

### 2. Membrane Associated Class II Protein Constructs

CIICs may be associated with lipid bilayers (e.g., artificial membranes or cell membranes) as integral membrane proteins when they comprise a MAS. A MAS of a CIIC may comprise either (i) an aa sequence that directly associates with the hydrophobic portion of the bilayers or membranes, or (ii) an aa sequence that leads to post-translational addition of groups (e.g., hydrocarbon chains of lipids) that interact with the hydrophobic portion of the bilayers or membranes.

A MAS that comprises an aa sequence that interacts with the lipid portion of a lipid bilayer may be, for example, a single or multiple transmembrane domain sequence, or an amphipathic a helix that partitions into a monolayer of a lipid bilayer. For example, in some embodiments, the anchor comprises the transmembrane domain of an MHC protein (e.g., a Class II α lpha subunit transmembrane domain), a glycophorin A transmembrane domain which can dimerize, or the transmembrane domain of small integral membrane protein 1 (SMIM1). Such MAS sequences may appear in a CIIC either in place of scaffold sequences, or in addition to a scaffold sequence such as an Ig Fc sequence. MASs are generally located at or near the C-terminus of the CIIC (e.g., on the C-terminal side of the α2 domain and any scaffold sequences that may be present in addition to the MAS. Exemplary amphipathic helices that partition into one leaflet of a lipid bilayer include those of cytidylyltransferase, ADP Ribosylation Factor, blood-clotting factor VIII, vinculin, and DnaA discussed below.

Post-translational modification sequences that lead to the addition of hydrophobic groups resulting in the association of CIIC with lipid bilayers include glycosylphosphatidylinositol modification sequences and prenylation sequences.

Where the CIIC is to be expressed on a cell surface, the sequence should be placed accordingly. For example, where anchoring is accomplished by a single transmembrane domain (e.g., as in the case of MHC Class II and glycophorin A transmembrane domains) with its N-terminus exposed on the cell surface, the single transmembrane domain should be placed C-terminal to the α2 domain and any membrane proximal sequence that follows it (e.g., placed C-terminal to it). Similarly, sequences leading to post-translational modification should be placed such that they do not disrupt the CIIC structure. Accordingly, post-translational modification sequences should be placed C-terminal to the α2 domain, for example, as part of, or following, a scaffold sequence.

CIICs may also be peripherally associated with cell membranes. Where CIICs are to be peripherally associated with a cell membrane, the sequences resulting in membrane association may be placed at any portion of the molecule provided they do not disrupt CIIC function. Accordingly, aa sequences leading to peripheral association with natural or artificial membranes may be placed C-terminal to the α2 domain, for example, as part of, or following, a scaffold sequence.

### B. Elements of Class II Protein Constructs

### 1. MHC Protein Sequences

The CIICs as described herein comprise MHC Class II sequences from any of a number of various species, including human MHC polypeptides (HLA polypeptides), rodent (e.g., mouse, rat, etc.) MHC polypeptides, and MHC polypeptides of other mammalian species (e.g., lagomorphs, non-human primates, canines, felines, ungulates (e.g., equines, bovines, ovines, caprines, etc.)), and the like. Typically, the CIICs described herein comprise human MHC Class II sequences. The CIICs as described herein may comprise human MHC Class II polypeptide sequences. The CIICs as described herein may comprise mouse MHC Class II polypeptide sequences.

As used herein, the term “Class II MHC polypeptide” refers to a Class II MHC α subunit (chain) polypeptide, a Class II MHC β subunit (chain) polypeptide, or only a portion of a Class II MHC α and/or β chain polypeptide, or combinations of the foregoing. For example, the term “Class II MHC polypeptide” as used herein can refer to a polypeptide that includes: i) only the α1 domain of a Class II MHC α chain; ii) only the α2 domain of a Class II MHC α chain; iii) only the α1 domain and the α2 domain of a Class II MHC α chain; iv) only the β1 domain of a Class II MHC β chain; v) only the β2 domain of a Class II MHC β chain; vi) only the β1 domain and the β2 domain of a Class II MHC β chain; vii) the α1 domain of a Class II MHC α chain, the β1 domain of a Class II MHC β chain, and the β2 domain of a Class II MHC; and the like. CIICs typically include the α1 and α2 domains of Class II MHC polypeptide α chains, and the β1 and β2 domains of class II MHC polypeptide β chains, which represent all or most of the extracellular class II protein required for presentation of an epitope. The α1 and α2 domain sequences may be followed by an α chain membrane proximal region. Similarly, the β1 and β2 domain sequences may be followed by a β chain membrane proximal region. Both the α and 1 Class II MHC polypeptide sequences may be of human origin.

As discussed above, where the CIICs and their higher order complexes (e.g., duplex CIICs) are intended to be soluble in aqueous media under physiological conditions (e.g., soluble in human blood plasma at therapeutic levels) they are not intended to include membrane anchoring domains (such as transmembrane regions of MHC Class II a or β chains) or a part thereof sufficient to anchor the CIIC molecules (e.g., more than 50% of the CIIC molecules) in the membrane of a cell (e.g., a eukaryotic cell such as a mammalian cell such as a Chinese Hamster Ovary or “CHO” cell) in which the CIIC is expressed. Similarly, unless expressly stated otherwise, the CIICs described herein are mature proteins that do not include the leader and/or intracellular portions (e.g., cytoplasmic tails) that may be present in some MHC Class II proteins.

In contrast, to soluble CIICs, where CIICs or their higher order complexes are intended to be membrane associated proteins they may include an aa sequence that result in post-translational modification (lipidation or prenylation), or one or more transmembrane domains as discussed above. For example, a CIIC may comprise a class II MHC transmembrane domain and optionally any intervening membrane proximal region. As the α2 domain sequence is located closest to the carboxyl terminus of a CIIC, the α2 domain or may be followed by, for example, its transmembrane domain, or its membrane proximal sequence and transmembrane domain as in the naturally occurring a chain.

Class II MHC aa sequences that may appear in CIICs include aa sequences from MHC Class II DP a (DPA) and 1 (DPB) subunits, DQ α (DQA) and β (DQB) subunits, and DR α (DRA) and β (DRB) subunits. The human MHC or HLA locus is highly polymorphic in nature, and thus as used herein the term “Class II MHC polypeptide” includes allelic forms of any known Class II MHC polypeptide. See, e.g., the HLA Nomenclature site run by the Anthony Nolan Research Institute, available on the world wide web at hla.alleles.org/nomenclature/index.html, which indicates that there are numerous DRA alleles, DRB1 alleles, DRB3 alleles, DRB4 alleles, DRB5 alleles, DRB6 alleles, DRB7 alleles, DRB9 alleles, DQA1 alleles, DQB1 alleles, DPA1, and DPB1 alleles.

Unless stated otherwise a CIIC may comprise Class II MHC α and β chain sequences, without the leader, transmembrane, and intracellular portions (e.g., cytoplasmic tails). Thus, a CIIC may comprise the α1, α2, β1, and β2 domains, and optionally the membrane proximal portions of Class II MHC α and β chains, but does not, unless stated otherwise, include any one or more of the leader, transmembrane, and intracellular portions (e.g., cytoplasmic tails) that may be present in a Class II MHC α chain. A linker sequence denoted “La” may be interposed between the α1 and α2 domains (see, e.g., FIG. 1, structure A). Similarly, a linker sequence denoted “Lp” may be interposed between the β1 and β2 domains (see, e.g., FIG. 1, structure A). The Class II MHC α chain sequences of a CIIC, and particularly the α1 and β1 domains, may include a variety of advantageous aa substitutions.

When addressing corresponding substitutions in, for example, different α1 or β1 domains of MHC sequences (e.g., different alleles), corresponding aas and aa positions in the sequences are determined by aligning the sequences. For MHC α subunit alignments the combined α1 and α2 domain sequences are aligned for the MHC α subunit comparisons. For MHC β subunit alignments the combined β1 and β2 domain sequences are aligned for MHC 1 subunit comparisons. Unless stated otherwise, sequence comparisons for determining corresponding substitutions are conducted using Clustal Omega Version 1.2.2 available on the world wide web at www.ebi.ac.uk/Tools/msa/clustalo/.

### a) MHC Class II Alpha Chains

MHC Class II α lpha subunits (chains) comprise an α1 domain and an α2 domain. In some cases, the α1 and α2 domain sequences present in an antigen-presenting cell are from the same MHC Class II α chain polypeptide (the sequence of the same allele). In some cases, the α1 and α2 domain sequences present in an antigen-presenting cell are from two different MHC Class II α chain polypeptides (alleles). FIGS. 4, 9, 11, and 12, present DR, DP, and DQ alpha chain α1 and α2 domain sequences along with their membrane proximal sequences, transmembrane domain sequences and intracellular domain sequences, but lacking their signal/leader sequence. Unless stated otherwise, the MHC Class II α chain sequences are numbered starting with the first amino acid of the α1 domain, i.e., the first amino acid following the signal sequence. In some instances, from 1 to 3 aas may be removed from the N-terminus of an MHC (e.g., HLA) Class II α1 domain as it appears in a CIIC. In such instances the numbering of the remaining aas of the α chain sequences does not change and can be determined by alignment with the corresponding unmodified MHC allele.

An MHC Class II α lpha chain sequence comprising the α1 and α2 domain sequences suitable for inclusion in a CIIC may have a length of from about 165 aas to about 210 aas (including any Lα linkers interposed between the α1 and α2 domains but excluding membrane proximal sequences), for example, an MHC Class II α lpha chain suitable for inclusion in a CIIC may have a length of from about 170 to about 190 aas or from about 175 to about 185 aas in length. An MHC Class II α1 domain suitable for inclusion in a CIIC may have a length of from about 75 aas to about 95 aas, for example, an MHC Class II α1 domain suitable for inclusion in a CIIC may have a length of from about 80 aas to about 90 aas, or from about 83 aas to about 88 aas. An MHC Class II α2 domain suitable for inclusion in a CIIC may have a length of from about 85 aas to about 105 aas, for example, an MHC Class II α2 domain suitable for inclusion in a CIIC may have a length of from about 90 aas to about 100 aas, or from about 92 aas to about 98 aas.

Where a Lα linker is present in a CIIC (other than a naturally occurring linker), the aa sequence of the linker is not included when determining percent sequence identity. Accordingly, the percent sequence identity between the α1 domain sequence or the α2 domain sequence present in the CIIC and the α1 domain sequence or the α2 domain sequence present in a specific allele may be assessed over a span of contiguous α1 or α2 domain sequence aas in the CIIC that do not include the Lα linker aa sequence. Likewise, the collective percent sequence identity between the α1 and α2 domain sequences present in the CIIC and the α1 and α2 domain sequences present in a specific allele is determined without reference to the Lα linker aa sequence.

An MHC class II α chain polypeptide suitable for inclusion in a CIIC may comprise a substitution of an aa in the last 11 aas, including e.g., the last 10 aas, of the MHC α subunit α1 domain sequence for forming a linker disulfide or body disulfide bond for stabilizing the CIIC.

Some cysteine residues in CIICs that are not part of a disulfide bond stabilizing a CIIC structure (unpaired cysteines), such as C47 of some DQA α1 domain sequences (e.g., DQA*05:01), can be linked to difficulties in expression. The presence of such unpaired cysteines may result in poor expression levels or the production of misfolded protein products. Substitution of such cysteines with an amino acid other than a cysteine (e.g., an amino acid other than cysteine or proline, such as serine, arginine, or lysine) can lead to higher levels of protein expression in the native state (e.g., non-denatured or non-aggregated) relative to the levels observed with the unpaired cysteine-containing molecule. Accordingly, unpaired cysteines appearing at, for example, aas 43 through 48 of the α chain polypeptide sequence (α1 and α2 domain sequence) may be substituted by an aa other than cysteine, or by an aa other than cysteine or proline. The cysteine substitutions may be made with serine, arginine, or lysine, or with serine, which is the amino acid closest to cysteine in size and other characteristics, but lacking the nucleophilicity of the cysteine thiol group.

### (1) DRA Polypeptides

A suitable MHC Class II DR α subunit (DRA) polypeptide for inclusion in a CIIC may have at least 85%, at least 90%, at least 95%, at least 98%, at least 99%, or 100% aa sequence identity with at least 150, at least 160, at least 165, or at least 170 contiguous aas of the α1 and α2 domain region of a DRA aa sequence depicted in FIG. 4 or a naturally occurring allelic variant thereof. In some cases, the DRA polypeptide has a length of about 178 aas, including, e.g., 175, 176, 177, 178, 179, 180, 181, 182, 183, 184, or 185 aas.

As used herein, the term “DRA polypeptide” includes allelic variants, e.g., naturally occurring allelic variants. Thus, in some cases, a suitable DRA polypeptide comprises aas 1-178 of DRA*01:02 (see FIG. 4), or an allelic variant thereof. In some cases, the allelic variant is the DRA*01:01 polypeptide (e.g., from the DRA*01:01:01:01 allele) that differs from DRA*01:02 by having a valine in place of the leucine at position 217 (see FIG. 4).

A suitable DRA aa sequence for inclusion in a CIIC may have at least 85%, at least 90%, at least 95%, at least 98%, at least 99%, or 100%, aa sequence identity with at least 160, at least 165, at least 170, or at least 175 contiguous aas of the α1 and α2 domain sequences of DRA*01:02 sequence depicted in FIG. 4. A suitable DRA aa sequence for inclusion in a CIIC may have at least 90% or 100% aa sequence identity to at least 165 contiguous aas of the DRA α1 and α2 domain sequence of DRA1*01:01 or DRA*01:02. A suitable DRA aa sequence for inclusion in a CIIC may have at least 95% or at least 98% aa sequence identity to at least 165 contiguous aas of the DRA α1 and α2 domain sequence of DRA1*01:01 or DRA*01:02.

Thus, a suitable DRA polypeptide may comprise an aa sequence having at least 90% or at least 95% aa sequence identity to at least 165 contiguous aas of the DRA*01:02 α1 and α2 domain sequences: IKEEH VIIQAEFYLN PDQSGEFMFD FDGDEIFHVD MAKKETVWRL EEFGRFASFE AQGALANIAV DKANLEIMTK RSNYTPITNV PPEVTVLTNS PVELREPNVL ICFIDKFTPP VVNVTWLRNG KPVTTGVSET VFLPREDHLF RKFHYLPFLP STEDVYDCRV EHWGLDEPLL KHW (SEQ ID NO:102, aas 1-178, see FIG. 4), or an allelic variant thereof. In some cases, a DRA polypeptide suitable for inclusion in a CIIC comprises an aa substitution, relative to a wild-type DRA polypeptide, where the amino acid substitution replaces an amino acid (other than a Cys) with a Cys (e.g., for forming a disulfide bond that stabilizes the CIIC).

A CIIC may comprise a variant DRA polypeptide that comprises a non-naturally occurring Cys residue (e.g., for forming a disulfide bond that stabilizes the CIIC). For example, a CIIC may comprise a variant DRA polypeptide comprising a Cys substituted for an aa at any of positions 74-76 for formation of a linker disulfide bond (e.g., a Cys substitution selected from T74C, K75C, or R76C (see, e.g., FIG. 4). A CIIC may comprise a variant DRA polypeptide comprising a Cys substituted for an aa at any of positions 80-82 for formation of a body disulfide bond (e.g., a Cys substitution selected from T80C, P81C, or 182C (see, e.g., FIG. 4). Separate from, or in addition to, substitutions introducing Cys residues for the formation of body or linker disulfide bonds,a CIIC containing DRA polypeptide sequences may comprise substitutions in the α1 domain sequence at one or more of positions 37, 44, 49 or 72 of HLA DRA*01:02, or the corresponding positions in other DRA alleles based upon sequence alignment. Position 37 of the α1 domain may be or be substituted by an acidic residue such as E or D (e.g., an Δ37E substitution in DRA*01:02), position 49 may be substitute by an H (e.g., a G49H substitution in DRA*01:02), position 72, which is an I in the wild-type sequence may remain an I or may be substituted by another aliphatic aa such as L or V, and position 44 of the DRA α1 domain sequence (i) is an aa other than cysteine, or (ii) when aa position 44 of the DRA α1 domain sequence is an arginine, may be substituted by a serine or lysine (e.g., a R44S or R44K substitution DRA*01:02).

A suitable DRA α1 domain sequence for inclusion in a CIIC may comprise an aa sequence having at least 85% (e.g., at least 90%, at least 95%, at least 98%) or 100% aa sequence identity to the aa sequence: IKEEVIIQAEFYLN PDQSGEFMFD FDGDEIFHVD MAKKETVWRL EEFGRFASFE AQGALANIAV DKANLEIMTK RSNYTPITN (SEQ ID NO:189), and optionally having a length of about 84 aas, including, e.g., 80, 81, 82, 83, 84, 85, or 86 aas. A suitable DRA α1 domain sequence may also have at least 90% or at least 95% aa sequence identity to SEQ ID NO:189.

A suitable DRA α2 domain sequence for inclusion in a CIIC may comprise an aa sequence having at least 85% (e.g., at least 90%, at least 95%, at least 98%) or 100% aa sequence identity to the aa sequence: V PPEVTVLTNS PVELREPNVL ICFIDKFTPP VVNVTWLRNG KPVTTGVSET VFLPREDHLF RKFHYLPFLP STEDVYDCRV EHWGLDEPLL KHW (SEQ ID NO:190), and optionally having a length of about 94 aas, including, e.g., 90, 91, 92, 93, 94, 95, 96, 97, or 98 aas. A suitable DRA α2 domain sequence may also have at least 90% or at least 95% aa sequence identity to SEQ ID NO:190.

### (2) DPA Polypeptides

A suitable MHC Class II DPA polypeptide for inclusion in a CIIC may have at least 85%, at least 90%, at least 95%, at least 98%, at least 99%, or 100% aa sequence identity with at least 150, at least 160, at least 165, or at least 170 contiguous aas of the α1 and α2 domain region of a DPA aa sequence depicted in FIG. 9 or a naturally occurring allelic variant thereof. In some cases, the DPA polypeptide has a length of about 181 aas, including, e.g., 175, 176, 177, 178, 179, 180, 181, 182, 183, 184, or 185 aas).

As used herein, the term “DPA polypeptide” includes allelic variants, e.g., naturally occurring allelic variants. Thus, in some cases, a suitable DPA polypeptide comprises aas 1-181 of DPA*01:03 (see FIG. 9), or an allelic variant thereof. In some cases, the allelic variant is the DPA*02:01 (see FIG. 9).

A suitable DPA aa sequence for inclusion in a CIIC may have at least 85%, at least 90%, at least 95%, at least 98%, at least 99%, or 100% aa sequence identity with at least 160, at least 165, at least 170, or at least 175 contiguous aas of the α1 and α2 domain sequences of DPA*01:03 or DPA1*02:01 sequence depicted in FIG. 9. A suitable DPA aa sequence for inclusion in a CIIC may have at least 90% or 100% aa sequence identity to at least 165 contiguous aas of the DPA α1 and α2 domain sequences of DPA1*01:03 or DPA*02:01. A suitable DPA aa sequence for inclusion in a CIIC polypeptide may have at least 95% or at least 98% aa sequence identity to at least 165 contiguous aas of the DPA α1 and α2 domain sequences of DPA1*01:03 or DPA*02:01.

Thus, a suitable DPA polypeptide may comprise an aa sequence having at least 90% or at least 95% aa sequence identity to at least 165 contiguous aas of the DPA*01:03 α1 and α2 domain sequences: AG AIKADHVSTY AAFVQTHRPT GEFMFEFDED EMFYVDLDKK ETVWHLEEFG QAFSFEAQGG LANIAILNNN LNTLIQRSNH TQATNDPPEV TVFPKEPVEL GQPNTLICHI DKFFPPVLNV TWLCNGELVT EGVAESLFLP RTDYSFHKFH YLTFVPSAED FYDCRVEHWG LDQPLLKHW (SEQ ID NO:103, aas 1-181, see FIG. 9), or an allelic variant thereof.

A CIIC may comprise a variant DPA polypeptide that comprises a non-naturally occurring Cys residue (e.g., for forming a disulfide bond that stabilizes the CIIC). For example, a CIIC may comprise a variant DPA polypeptide comprising a Cys substituted for an aa at any of positions 77-79 for formation of a linker disulfide bond (e.g., a Cys substitution selected from 177C, Q78C, or R79C (see, e.g., FIG. 9). A CIIC may comprise a variant DPA polypeptide comprising a Cys substituted for an aa at any of positions 83-85 for formation of a body disulfide bond (e.g., a Cys substitution selected from T83C, Q84C, or Δ85C (see, e.g., FIG. 9).

Separate from, or in addition to, substitutions introducing Cys residues for the formation of body or linker disulfide bonds, a CIIC containing DPA polypeptide sequences may comprise substitutions in the α1 domain sequence at one or more of positions 40, 47, 52 or 75 of HLA DPA1*01:03, or the corresponding positions in other DPA alleles based upon sequence alignment. Position 40 of the α1 domain may be substituted by an acidic residue such as E or D (e.g., an Δ37E substitution in DPA*01:03), position 52 may be substituted by an H (e.g., a G49H substitution in DPA*01:03), position 75, may be substituted by an aliphatic aa such as I, L, or V (e.g., a T751 substitution in DPA*01:03), and position 47 of the DPA α1 domain sequence (i) is an aa other than cysteine, or (ii) when aa position 47 of the DPA α1 domain sequence is an His, may be substituted by a serine or lysine (e.g., a H47S or H47K substitution DPA*01:03).

A suitable DPA α1 domain sequence for inclusion in a CIIC polypeptide may comprise an aa sequence having at least 85% (e.g., at least 90%, at least 95%, at least 98%) or 100% aa sequence identity to the aa sequence: AGAIKADHVSTY AAFVQTHRPT GEFMFEFDED EMFYVDLDKK ETVWHLEEFG QAFSFEAQGG LANIAILNNN LNTLIQRSNH TQATN (SEQ ID NO:191), and optionally having a length of about 87 aas, including, e.g., 84, 85, 86, 87, 88, or 89 aas. A suitable DPA α1 domain sequence may also have at least 90% or at least 95% aa sequence identity to SEQ ID NO:191.

A suitable DPA α2 domain sequence for inclusion in a CIIC polypeptide may comprise an aa sequence having at least 85% (e.g., at least 90%, at least 95%, at least 98%) or 100% aa sequence identity to the aa sequence: DPPEV TVFPKEPVEL GQPNTLICHI DKFFPPVLNV TWLCNGELVT EGVAESLFLP RTDYSFHKFH YLTFVPSAED FYDCRVEHWG LDQPLLKHW (SEQ ID NO:192), and optionally having a length of about 94 aas, including, e.g., 91, 92, 93, 94, 95, 96, or 97 aas. A suitable DPA α2 domain sequence may also have at least 90% or at least 95% aa sequence identity to (SEQ ID NO:192).

### (3) DQA Polypeptides

### (a) DQA1 Polypeptides

A suitable MHC Class II DQA1 polypeptide for inclusion in a CIIC may have at least 85%, at least 90%, at least 95%, at least 98%, at least 99%, or 100% aa sequence identity with at least 150, at least 160, at least 165, or at least 170 contiguous aas of the α1 and α2 domain region of a DQA1 aa sequence depicted in FIG. 11 or a naturally occurring allelic variant thereof. In some cases, the DQA1 polypeptide has a length of about 181 aas, including, e.g., 175, 176, 177, 178, 179, 180, 181, 182, 183, 184, or 185 aas.

As used herein, the term “DQA1 polypeptide” includes allelic variants, e.g., naturally occurring allelic variants. Thus, in some cases, a suitable DQA1 polypeptide comprises aas 1-181 of DQA1*01:01 (see FIG. 11), or an allelic variant thereof. In some cases, the allelic variant is DQA1*05:01 (see FIG. 11).

A suitable DQA1 aa sequence for inclusion in a CIIC polypeptide may have at least 85%, at least 90%, at least 95%, at least 98%, at least 99%, or 100% aa sequence identity with at least 160, at least 165, at least 170, or at least 175 contiguous aas of the α1 and α2 domain sequences of the DQA1*01:01 or DQA1*05:01 sequence depicted in FIG. 11. A suitable DQA1 aa sequence for inclusion in a CIIC polypeptide may have at least 90% or 100% aa sequence identity to at least 165 contiguous aas of the DQA1 α1 and α2 domain sequence of DQA1*01:01 or DQA1*05:01. A suitable DQA1 aa sequence for inclusion in a CIIC polypeptide may have at least 95% or at least 98% aa sequence identity to at least 165 contiguous aas of the DQA1 α1 and α2 domain sequence of DQA1*01:01 or DQA1*05:01. Thus, a suitable DQA1 polypeptide may comprise an aa sequence having at least 90% or at least 95% aa sequence identity to at least 165 contiguous aas of the DQA1*01:01 α1 and α2 domain sequence aas 1 through 181 (see FIG. 11). Alternatively, a suitable DQA1 polypeptide may comprise an aa sequence having at least 90% or at least 95% aa sequence identity to at least 165 contiguous aas of the DQA1*05:01 α1 and α2 domain sequence aas 1 through 181 (see FIG. 11).

A CIIC may comprise a variant DQA1 polypeptide that comprises a non-naturally occurring Cys residue (e.g., for forming a disulfide bond that stabilizes the CIIC). For example, a CIIC may comprise a variant DQA1 polypeptide comprising a Cys substitution for formation of a linker disulfide bond at any one of the aas in the sequence “IKR” (see positions 76-78 or 77-79 depending on the allele depicted in FIG. 11, e.g., a Cys substitution selected from 177C, K78C, or R79C (see, e.g., FIG. 11). A CIIC may comprise a variant DQA1 polypeptide comprising a Cys substitution for forming a body disulfide bond at any one of the aas in the sequence “TAA” (see positions 82-84 or 83-85 depending on the allele depicted in FIG. 11 (e.g., a Cys substitution selected from T82C, A83C, or Δ84C in FIG. 11).

Separate from, or in addition to, substitutions introducing Cys residues for the formation of body or linker disulfide bonds, a CIIC containing DQA1 polypeptide sequences may comprise substitutions in the α1 domain sequence at one or more of positions 40, 47, 52 or 75 of HLA DQA1*01:01, or the corresponding positions in other DQA1 alleles based upon sequence alignment. Position 40 of the α1 domain may be substituted by an acidic residue such as E or D (e.g., a G40E substitution in DQA1*05:01), position 52 may be substitute by an H (e.g., a R52H substitution in In DQA1*05:01), position 75 may be substituted by an aliphatic aa such as I, L, or V (e.g., a S741 substitution in DQA1*05:01), and position 47 of the DQA1 α1 domain sequence (i) is an aa other than cysteine, or (ii) when aa position 47 of the DQA1 α1 domain sequence is a Cys, may be substituted by S or K (e.g., a C47S or C47K substitution in DQA1*05:01).

A suitable DQA1 α1 domain for inclusion in a CIIC polypeptide may comprise an aa sequence having at least 85% (e.g., at least 90%, at least 95%, at least 98%) or 100% aa sequence identity to aas 1-85 of any of the DQA1 alleles provided in FIG. 11, and optionally having a length of about 86 aas, including, e.g., 84, 85, 86, 87, 88, or 89 aas. A suitable DQA1 α1 domain for inclusion in a CIIC polypeptide may comprise an aa sequence having at least 90% or at least 95% aa sequence identity to aas 1-85 of any of the DQA1 alleles provided in FIG. 11, and optionally having a length of about 86 aas, including, e.g., 84, 85, 86, 87, 88, or 89 aas. A suitable DQA1 α1 domain for inclusion in a CIIC polypeptide may comprise an aa sequence having at least 90% or at least 95% aa sequence identity to aas 1-85 of DQA1*01:01, and optionally having a length of about 86 aas, including, e.g., 84, 85, 86, 87, 88, or 89 aas. A suitable DQA1 α1 domain for inclusion in a CIIC polypeptide may comprise an aa sequence having at least 90% or at least 95% aa sequence identity to aas 1-85 of DQA1*05:01, and optionally having a length of about 85 aas, including, e.g., 83, 84, 85, 86, 87, or 88 aas.

A suitable DQA1 α2 domain for inclusion in a CIIC polypeptide may comprise an aa sequence having at least 85% (e.g., at least 90%, at least 95%, at least 98%) or 100% aa sequence identity to aas 87-180 of any of the DQA1 alleles provided in FIG. 11, and optionally having a length of about 93 aas, including, e.g., 91, 92, 93, 94, 95, 96, or 97 aas. A suitable DQA1 α2 domain for inclusion in a CIIC polypeptide may comprise an aa sequence having at least 90% or at least 95% aa sequence identity to aas 87-180 of any of the DQA1 alleles provided in FIG. 11, and optionally having a length of about 93 aas, including, e.g., 91, 92, 93, 94, 95, 96, or 97 aas. A suitable DQA1 α2 domain for inclusion in a CIIC polypeptide may comprise an aa sequence having at least 90% or at least 95% aa sequence identity to aas 87-180 of DQA1*01:01, and optionally having a length of about 93 aas, including, e.g., 91, 92, 93, 94, 95, 96, or 97 aas. A suitable DQA1 α2 domain for inclusion in a CIIC polypeptide may comprise an aa sequence having at least 90% or at least 95% aa sequence identity to aas 87-180 of DQA1*05:01, and optionally having a length of about 93 aas, including, e.g., 91, 92, 93, 94, 95, 96, or 97 aas.

### (b) DQA2 Polypeptides

A suitable MHC Class II DQA2 polypeptide for inclusion in a CIIC may have at least 85%, at least 90%, at least 95%, at least 98%, at least 99%, or 100% aa sequence identity with at least 150, at least 160, at least 165, or at least 170 contiguous aas of the α1 and α2 domain region of a DQA2 aa sequence depicted in FIG. 12 or a naturally occurring allelic variant thereof. In some cases, the DQA2 polypeptide has a length of about 181 aas, including, e.g., 178, 179, 180, 181, 182, 183, 184, or 185 aas. As used herein, the term “DQA2 polypeptide” includes allelic variants, e.g., naturally occurring allelic variants. Thus, in some cases, a suitable DQA2 polypeptide comprises aas 1-181 of DQA2*01:01 (see FIG. 12).

A suitable DQA2 aa sequence for inclusion in a CIIC polypeptide may have at least 85% or at least 90% aa sequence identity with at least 165, at least 170, or at least 175 contiguous aas of the α1 and α2 domain sequences of DQA2*01:01 sequence depicted in FIG. 12. A suitable DQA2 aa sequence for inclusion in a CIIC polypeptide may have at least 90% or 100% aa sequence identity to at least 165 contiguous aas of the DQA2 α1 and α2 domain sequence of DQA2*01:01. A suitable DQA2 aa sequence for inclusion in a CIIC polypeptide may have at least 95% or at least 98% aa sequence identity to at least 165 contiguous aas of the DQA2 α1 and α2 domain sequence of DQA2*01:01.

Thus, a suitable DQA2 polypeptide may comprise an aa sequence having at least 90% or at least 95% aa sequence identity to at least 165 contiguous aas of the DQA2*01:01 α1 and α2 domain sequences: EDIVADH VASYGVNFYQ SHGPSGQYTH EFDGDEEFYV DLETKETVWQ LPMFSKFISF DPQSALRNMA VGKHTLEFMM RQSNSTAATN EVPEVTVFSK FPVTLGQPNT LICLVDNIFP PVVNITWLSN GHSVTEGVSE TSFLSKSDHS FFKISYLTFL PSADEIYDCK VEHWGLDEPL LKHW (SEQ ID NO:193, aas 1-181 of SEQ ID NO:88 see FIG. 12), or an allelic variant thereof. In some cases, a DQA2 polypeptide suitable for inclusion in a CIIC comprises an aa substitution, relative to a wild-type DQA2 polypeptide, where the amino acid substitution replaces an amino acid (other than a Cys) with a Cys (e.g., for forming a disulfide bond that stabilizes the CIIC).

A CIIC may comprise a variant DQA2 polypeptide that comprises a non-naturally occurring Cys residue (e.g., for forming a disulfide bond that stabilizes the CIIC). For example, a CIIC may comprise a variant DQA2 polypeptide comprising a Cys substituted for an aa at any of positions 77-79 for formation of a linker disulfide bond (e.g., a Cys substitution selected from M77C, R78C, or Q79C (see, e.g., FIG. 12). A CIIC may comprise a variant DQA2 polypeptide comprising a Cys substituted for an aa at any of positions 83-85 for formation of a body disulfide bond (e.g., a Cys substitution selected from T83C, Δ84C, or Δ85C (see, e.g., FIG. 12). Separate from, or in addition to, substitutions introducing Cys residues for the formation of body or linker disulfide bonds, CIIC containing DQA2 polypeptide sequences may comprise substitutions in the α1 domain sequence at one or more of positions 40, 47, 52 or 75 of HLA DQA2*01:01, or the corresponding positions in other DQA2 alleles based upon sequence alignment. Position 40 of the α1 domain may be substituted by an acidic residue such as E or D (e.g., an Δ40E substitution in DQA2*01:01), position 52 may be substituted by an H (e.g., a S52H substitution in DQA2*01:01), position 75 may be substituted by an aliphatic aa such as I, L, or V (e.g., a F751 substitution in DQA2*01:01), and position 47 of the DQA2 α1 domain sequence (i) is an aa other than cysteine, or (ii) when aa position 47 of the DQA2 α1 domain sequence is a Cys, may be substituted by S or K (e.g., a C47S or C47K substitution in DQA2*01:01).

A suitable DQA2 α1 domain sequence, including-naturally occurring allelic variants thereof, may comprise an aa sequence having at least 85%, at least 90%, at least 95%, at least 98%, at least 99%, or 100% aa sequence identity to aas 1-86 of HLA DQA2*01:01, and optionally has a length of about 86 aas, including, e.g., 84, 85, 86, 87, 88, or 89 aas. A suitable DQA2 α1 domain sequence may also have at least 90% or at least 95% aa sequence identity to aas 1-86 of HLA DQA2*01:01.

A suitable DQA2 α2 domain sequence, including-naturally occurring allelic variants thereof, may comprise an aa sequence having at least 85%, at least 90%, at least 95%, at least 98%, at least 99%, or 100% aa sequence identity to aas 87-181 of HLA DQA2*01:01, and optionally has a length of about 94 aas, including, e.g., 91, 92, 93, 94, 95, 96, or 97 aas. A suitable DQA2 α2 domain sequence may also have at least 90% or at least 95% aa sequence identity to aas 87-181 of HLA DQA2*01:01.

### b) MHC Class II Beta Chains

MHC Class II beta subunits (chains) comprise a β1 domain and a β2 domain. In some cases, the β1 and β2 domain sequences present in an antigen-presenting cell are from the same MHC Class II β chain polypeptide. In some cases, the β1 and β2 domain sequences present in an antigen-presenting cell are from two different MHC Class II β chain polypeptides. FIGS. 5-8, 10, and 13-14 present DR, DP and DQ beta chain β1 and β2 domain sequences along with their membrane proximal sequences, but lacking their signal/leader, transmembrane domain, and intracellular domain sequences. Unless stated otherwise, the MHC Class II β chain sequences are numbered starting with the first amino acid of the β1 domain, i.e., the first amino acid following the signal sequence. In some instances, from 1 to 3 aas may be removed from the N-terminus of an MHC (e.g., HLA) Class II β1 domain as it appears in a CIIC. In such instances the numbering of the remaining aas of the β1 domain does not change and can be determined by alignment with the corresponding unmodified MHC allele.

An MHC Class II beta chain sequence comprising the β1 and β2 domain sequences suitable for inclusion in a CIIC may have a length of from about 165 aas to about 210 aas (including any Lβ linkers interposed between the 11 and 12 domains but excluding membrane proximal sequences). For example, an MHC Class II beta chain suitable for inclusion in a CIIC may have a length of from about 170 to about 200 aas or from about 180 to about 195 aas in length. An MHC Class II β1 domain suitable for inclusion in a CIIC may have a length of from about 85 aas to about 105 aas, for example, an MHC Class II β1 domain suitable for inclusion in a CIIC may have a length of from about 90 aas to about 100 aas, or from about 93 aas to about 98 aas. An MHC Class II β2 domain suitable for inclusion in a CIIC may have a length of from about 80 aas to about 105 aas, for example, an MHC Class II β2 domain suitable for inclusion in a CIIC may have a length of from about 85 aas to about 100 aas, or from about 90 aas to about 98 aas.

Where an Lβ linker (other than a naturally occurring linker) is present in a CIIC, the aa sequence of the linker is not included when determining percent sequence identity.

An MHC class II β chain polypeptide suitable for inclusion in a CIIC may comprise an aa substitution for forming a body disulfide bond, where the aa substitution replaces any one of aas 1-8 (e.g., aas 4-8 as shown in FIGS. 5-8, 10, and 13-14) of the β1 domain with a Cys. For example, in some cases, the MHC Class II β chain polypeptide is a variant DRB1 CIIC that comprises a P5C or F7C substitution.

### (1) DRB Peptides

MHC Class II DRB polypeptides for inclusion in a CIIC have both β1 and β2 domains. Some non-limiting examples of DRB1, DRB3, and DRB4 polypeptides are provided in FIGS. 5-8. The β1 and β2 domains of the DRB proteins shown in those figures are typically about 188 aas in length, with aas 1-95 making up the β1 domain and aas 96-188 making up the β2 domain. Aas 189-198 make up the membrane proximal region.

### (a) DRB1 Polypeptides

A suitable MHC Class II DRB1 polypeptide for inclusion in a CIIC may have at least 85%, at least 90%, at least 95%, at least 98%, at least 99%, or 100% aa sequence identity with at least 165, at least 170, at least 175, at least 180, or at least 185 contiguous aas of the β1 and β2 domain regions of a DRB1 aa sequence depicted in FIG. 5 or a naturally occurring allelic variant thereof. In some cases, the DRB1 polypeptide has a length of about 188 aas, including, e.g., 178, 179, 180, 181, 182, 183, 184, 185, 186, 187, or 188 aas. As used herein, the term “DRB1 polypeptide” includes allelic variants, e.g., naturally occurring allelic variants. Thus, in some cases, a suitable DRB1 polypeptide comprises a sequence that comprises aas 1-188 of DRB1*01:01 (see FIG. 5) or an allelic variant thereof. In some cases, the allelic variant is the DRB1*04:01 (see FIG. 5).

A suitable DRB1 aa sequence for inclusion in a CIIC polypeptide may have at least 85%, at least 90%, at least 95%, at least 98%, at least 99%, or 100% aa sequence identity with at least 165, at least 170, at least 175, at least 180, or at least 185 contiguous aas of the β1 and β2 domain sequences of the DRB1*01:01 or DRB1*04:01 sequences depicted in FIG. 5. A suitable DRB1 aa sequence for inclusion in a CIIC polypeptide may have at least 90% or 100% aa sequence identity to at least 170 contiguous aas of the DRB1 β1 and β2 domain sequences of DRB1*01:01 or DRB1*04:01. A suitable DRB1 aa sequence for inclusion in a CIIC polypeptide may have at least 95% or at least 98% aa sequence identity to at least 170 contiguous aas of the DRB1 β1 and β2 domain sequences of DRB1*01:01 or DRB1*04:01. Thus, a suitable DRB1 polypeptide may comprise an aa sequence having at least 95% or at least 98% aa sequence identity to at least 170 or at least 180 contiguous aas of the DRB1*01:01 β1 and β2 domain sequence aas 1 through 188 (see FIG.). Alternatively, a suitable DRB1 polypeptide may comprise an aa sequence having at least 95% or at least 98% aa sequence identity to at least 170 or at least 180 contiguous aas of the DRB1*04:01 β1 and β2 domain sequence aas 1 through 188 (see FIG. 5).

A CIIC may comprise a variant DRB1 polypeptide that comprises a non-naturally occurring Cys residue (e.g., for forming a body disulfide bond that stabilizes the CIIC). For example, a CIIC may comprise a variant DRB1 polypeptide comprising a Cys substitution for formation of a body disulfide bond at any one of aas 1-8 of the DRB1 sequences shown in FIG. 5. Alternatively, a CIIC may comprise a Cys substitution for formation of a body disulfide bond at any one of aas 5-8 of the DRB1 sequences shown in FIG. 5. Accordingly, a suitable DRB1 aa sequence for inclusion in a CIIC polypeptide may have at least 90% or at least 95% aa sequence identity to at least 170 contiguous aas of a DRB1 β1 and β2 domain sequences provided in FIG. 5, wherein the β1 sequence comprises a cysteine as a substitution for one of the aas in the subsequence PRFL (SEQ ID NO:194) (e.g., as a P5C or an F7C substitution). In one instance the DRB1 sequence is DRB1*01:01. In another instance, the DRB1 sequence is DRB1*04:01.

A suitable DRB1 β1 domain for inclusion in a CIIC polypeptide may comprise an aa sequence having at least 85% (e.g., at least 90%, at least 95%, at least 98%) or 100% aa sequence identity to aas 1-95 of any of the DRB1 alleles provided in FIG. 5, and optionally having a length of about 95 aas, including, e.g., 89, 90, 91, 92, 93, 94, 95, 96, 97, or 98 aas. A suitable DRB1 β1 domain for inclusion in a CIIC polypeptide may comprise an aa sequence having at least 90% or at least 95% aa sequence identity to aas 1-95 of any of the DRB1 alleles provided in FIG. 5, and optionally having a length of about 95 aas, including, e.g., 89, 90, 91, 92, 93, 94, 95, 96, 97, or 98 aas. A suitable DRB1 β1 domain for inclusion in a CIIC polypeptide may comprise an aa sequence having at least 90% or at least 95% aa sequence identity to aas 1-88 of DRB1*01:01, and optionally having a length of about 95 aas, including, e.g., 89, 90, 91, 92, 93, 94, 95, 96, 97, or 98 aas. A suitable DRB1 β1 domain for inclusion in a CIIC polypeptide may comprise an aa sequence having at least 90% or at least 95% aa sequence identity to aas 1-88 of DRB1*04:01, and optionally having a length of about 95 aas, including, e.g., 89, 90, 91, 92, 93, 94, 95, 96, 97, or 98 aas.

A suitable DRB1 β2 domain for inclusion in a CIIC polypeptide may comprise an aa sequence having at least 85% (e.g., at least 90%, at least 95%, at least 98%) or 100% aa sequence identity to aas 96-188 of any of the DRB1 alleles provided in FIG. 5, and optionally having a length of about 93 aas, including, e.g., 87, 88, 89, 90, 91, 92, 93, 94, 95, or 96 aas. A suitable DRB1 β2 domain for inclusion in a CIIC polypeptide may comprise an aa sequence having at least 90% or at least 95% aa sequence identity to aas 96-188 of any of the DRB1 alleles provided in FIG. 5, and optionally having a length of about 93 aas, including, e.g., 87, 88, 89, 90, 91, 92, 93, 94, 95, or 96 aas. A suitable DRB1 β2 domain for inclusion in a CIIC polypeptide may comprise an aa sequence having at least 90% or at least 95% aa sequence identity to aas 96-188 of DRB1*01:01, and optionally having a length of about 93 aas, including, e.g., 87, 88, 89, 90, 91, 92, 93, 94, 95, or 96 aas. A suitable DRB1 β2 domain for inclusion in a CIIC polypeptide may comprise an aa sequence having at least 90% or at least 95% aa sequence identity to aas 96-188 of DRB1*04:01, and optionally having a length of about 93 aas, including, e.g., 87, 88, 89, 90, 91, 92, 93, 94, 95, or 96 aas.

### (b) DRB3 Polypeptides

A suitable MHC Class II DRB3 polypeptide for inclusion in a CIIC may have at least 85%, at least 90%, at least 95%, at least 98%, at least 99%, or 100% aa sequence identity with at least 165, at least 170, at least 175, at least 180, or at least 185 contiguous aas of the β1 and β2 domain region of a DRB3 aa sequence depicted in FIG. 6 or a naturally occurring allelic variant thereof. In some cases, the DRB3 polypeptide has a length of about 188 aas, including, e.g., 178, 179, 180, 181, 182, 183, 184, 185, 186, 187, or 188 aas. As used herein, the term “DRB3 polypeptide” includes allelic variants, e.g., naturally occurring allelic variants. Thus, in some cases, a suitable DRB3 polypeptide comprises a sequence that comprises aas 1-188 of DRB3*01:01 (see FIG. 6), or an allelic variant thereof. In some cases, the allelic variant is the DRB3*02:01 or DRB3*03:01 (see FIG. 6).

A suitable DRB3 aa sequence for inclusion in a CIIC polypeptide may have at least 85%, at least 90%, at least 95%, at least 98%, at least 99%, or 100% aa sequence identity with at least 165, at least 170, at least 175, at least 180, or at least 185 contiguous aas of the β1 and β2 domain sequences of the DRB3*01:01, DRB3*02:01, or DRB3*03:01 sequences depicted in FIG. 6. A suitable DRB3 aa sequence for inclusion in a CIIC polypeptide may have at least 90% or 100% aa sequence identity to at least 170 contiguous aas of the DRB3 β1 and β2 domain sequences of DRB3*01:01 or DRB3*02:01. A suitable DRB3 aa sequence for inclusion in a CIIC polypeptide may have at least 95% or at least 98% aa sequence identity to at least 170 contiguous aas of the DRB3 β1 and β2 domain sequences of DRB3*01:01 or DRB3*02:01. Thus, a suitable DRB3 polypeptide may comprise an aa sequence having at least 95% or at least 98% aa sequence identity to at least 170 or at least 180 contiguous aas of the DRB3*01:01 β1 and β2 domain sequence aas 1 through 188 (see FIG. 6). Alternatively, a suitable DRB3 polypeptide may comprise an aa sequence having at least 95% aa sequence identity to at least 170 or at least 180 contiguous aas of the DRB3*02:01 β1 and β2 domain sequence aas 1 through 188 (see FIG. 6).

A CIIC may comprise a variant DRB3 polypeptide that comprises a non-naturally occurring Cys residue (e.g., for forming a body disulfide bond that stabilizes the CIIC). For example, a CIIC may comprise a variant DRB3 polypeptide comprising a Cys substitution for formation of a body disulfide bond at any one of aas 1-8 of the DRB3 sequences shown in FIG. 6. Alternatively, a CIIC may comprise a Cys substitution for formation of a body disulfide bond at any one of aas 5-8 of the DRB3 sequences shown in FIG. 6. Accordingly, a suitable DRB3 aa sequence for inclusion in a CIIC polypeptide may have at least 90% or at least 95% aa sequence identity to at least 170 contiguous aas of a DRB3 β1 and β2 domain sequence provided in FIG. 6, wherein the β1 sequence comprises a cysteine as a substitution for one of the aas in the subsequence PRFL (SEQ ID NO:194) (e.g., as a P5C or an F7C substitution). In one instance the DRB3 sequence is DRB3*01:01. In another instance, the DRB3 sequence is DRB3*02:01 or DRB3*03:01.

A suitable DRB3 β1 domain for inclusion in a CIIC polypeptide may comprise an aa sequence having at least 85% (e.g., at least 90%, at least 95%, at least 98%) or 100% aa sequence identity to aas 1-95 of any of the DRB3 alleles provided in FIG. 6, and optionally having a length of about 95 aas, including, e.g., 89, 90, 91, 92, 93, 94, 95, 96, 97, or 98 aas. A suitable DRB3 β1 domain for inclusion in a CIIC polypeptide may comprise an aa sequence having at least 90% or at least 95% aa sequence identity to aas 1-95 of any of the DRB3 alleles provided in FIG. 6, and optionally having a length of about 95 aas, including, e.g., 89, 90, 91, 92, 93, 94, 95, 96, 97, or 98 aas.

A suitable DRB3 β1 domain for inclusion in a CIIC polypeptide may comprise an aa sequence having at least 90%, or at least 95% aa sequence identity to aas 1-88 of DRB3*01:01, and optionally having a length of about 95 aas, including, e.g., 89, 90, 91, 92, 93, 94, 95, 96, 97, or 98 aas. A suitable DRB3 β1 domain for inclusion in a CIIC polypeptide may comprise an aa sequence having at least 90%, or at least 95% aa sequence identity to aas 1-88 of DRB3*02:01, and optionally having a length of about 95 aas, including, e.g., 89, 90, 91, 92, 93, 94, 95, 96, 97, or 98 aas. A suitable DRB3 β1 domain for inclusion in a CIIC polypeptide may comprise an aa sequence having at least 90% or at least 95% aa sequence identity to aas 1-88 of DRB3*03:01, and optionally having a length of about 95 aas, including, e.g., 89, 90, 91, 92, 93, 94, 95, 96, 97, or 98 aas.

A suitable DRB3 β2 domain for inclusion in a CIIC polypeptide may comprise an aa sequence having at least 85% (e.g., at least 90%, at least 95%, at least 98%) or 100% aa sequence identity to aas 96-188 of any of the DRB3 alleles provided in FIG. 6, and optionally having a length of about 93 aas, including, e.g., 87, 88, 89, 90, 91, 92, 93, 94, 95, or 96 aas. A suitable DRB3 β2 domain for inclusion in a CIIC polypeptide may comprise an aa sequence having at least 90% or at least 95% aa sequence identity to aas 96-188 of any of the DRB3 alleles provided in FIG. 6, and optionally having a length of about 93 aas, including, e.g., 87, 88, 89, 90, 91, 92, 93, 94, 95, or 96 aas. A suitable DRB3 β2 domain for inclusion in a CIIC polypeptide may comprise an aa sequence having at least 90% or at least 95% aa sequence identity to aas 96-188 of DRB3*01:01, and optionally having a length of about 93 aas, including, e.g., 87, 88, 89, 90, 91, 92, 93, 94, 95, or 96 aas. A suitable DRB3 β2 domain for inclusion in a CIIC polypeptide may comprise an aa sequence having at least 90% or at least 95% aa sequence identity to aas 96-188 of DRB3*02:01, and optionally having a length of about 93 aas, including, e.g., 87, 88, 89, 90, 91, 92, 93, 94, 95, or 96 aas. A suitable DRB3 β2 domain for inclusion in a CIIC polypeptide may comprise an aa sequence having at least 90% or at least 95% aa sequence identity to aas 96-188 of DRB3*03:01, and optionally having a length of about 93 aas, including, e.g., 87, 88, 89, 90, 91, 92, 93, 94, 95, or 96 aas.

### (c) DRB4 Polypeptides

A suitable MHC Class II DRB4 polypeptide for inclusion in a CIIC may have at least 85%, at least 90%, at least 95%, at least 98%, at least 99%, or 100% aa sequence identity with at least 165, at least 170, at least 175, at least 180, or at least 185 contiguous aas of the β1 and β2 domain regions of a DRB4 aa sequence depicted in FIG. 7 or a naturally occurring allelic variant thereof. In some cases, the DRB4 polypeptide has a length of about 188 aas, including, e.g., 178, 179, 180, 181, 182, 183, 184, 185, 186, 187, or 188 aas. As used herein, the term “DRB4 polypeptide” includes allelic variants, e.g., naturally occurring allelic variants. Thus, in some cases, a suitable DRB4 polypeptide comprises a sequence that comprises aas 1-188 of DRB4*01:01 (see FIG. 7), or an allelic variant thereof. In some cases, the allelic variant is the DRB4*01:03 (see FIG. 7).

A suitable DRB4 aa sequence for inclusion in a CIIC polypeptide may have at least 85%, at least 90%, at least 95%, at least 98%, at least 99%, or 100% aa sequence identity with at least 165, at least 170, at least 175, at least 180, or at least 185 contiguous aas of the β1 and β2 domain sequences of the DRB4*01:01 or DRB4*01:03 sequences depicted in FIG. 7. A suitable DRB4 aa sequence for inclusion in a CIIC polypeptide may have at least 90% or 100% aa sequence identity to at least 170 contiguous aas of the DRB4 β1 and β2 domain sequences of DRB4*01:01 or DRB4*01:03. A suitable DRB4 aa sequence for inclusion in a CIIC polypeptide may have at least 95% or at least 98% aa sequence identity to at least 170 contiguous aas of the DRB4 β1 and β2 domain sequences of DRB4*01:01 or DRB4*01:03. Thus, a suitable DRB4 polypeptide may comprise an aa sequence having at least 95% aa sequence identity to at least 170 or at least 180 contiguous aas of the DRB4*01:01 β1 and β2 domain sequence aas 1 through 188 (see FIG. 7). Alternatively, a suitable DRB4 polypeptide may comprise an aa sequence having at least 95% aa sequence identity to at least 170 or at least 180 contiguous aas of the DRB4*01:03 β1 and β2 domain sequence aas 1 through 188 (see FIG. 7).

A CIIC may comprise a variant DRB4 polypeptide that comprises a non-naturally occurring Cys residue (e.g., for forming a body disulfide bond that stabilizes the CIIC). For example, a CIIC may comprise a variant DRB4 polypeptide comprising a Cys substitution for formation of a body disulfide bond at any one of aas 1-8 of the DRB4 sequences shown in FIG. 7. Alternatively, a CIIC may comprise a Cys substitution for formation of a body disulfide bond at any one of aas 5-8 of the DRB4 sequences shown in FIG. 7. Accordingly, a suitable DRB4 aa sequence for inclusion in a CIIC polypeptide may have at least 90% or at least 95% aa sequence identity to at least 170 contiguous aas of a DRB4 β1 and β2 domain sequence provided in FIG. 7, wherein the β1 sequence comprises a cysteine as a substitution for one of the aas in the subsequence PRFL (SEQ ID NO:194) (e.g., as a P5C or an F7C substitution). In one instance the DRB4 sequence is DRB4*01:01. In another instance, the DRB4 sequence is DRB4*01:03.

A suitable DRB4 β1 domain for inclusion in a CIIC polypeptide may comprise an aa sequence having at least 85% (e.g., at least 90%, at least 95%, at least 98%) or 100% aa sequence identity to aas 1-95 of any of the DRB4 alleles provided in FIG. 7, and optionally having a length of about 95 aas, including, e.g., 89, 90, 91, 92, 93, 94, 95, 96, 97, or 98 aas. A suitable DRB4 β1 domain for inclusion in a CIIC polypeptide may comprise an aa sequence having at least 90% or at least 95% aa sequence identity to aas 1-95 of any of the DRB4 alleles provided in FIG. 7, and optionally having a length of about 95 aas, including, e.g., 89, 90, 91, 92, 93, 94, 95, 96, 97, or 98 aas. A suitable DRB4 β1 domain for inclusion in a CIIC polypeptide may comprise an aa sequence having at least 90% or at least 95% aa sequence identity to aas 1-88 of DRB4*01:01, and optionally having a length of about 95 aas, including, e.g., 89, 90, 91, 92, 93, 94, 95, 96, 97, or 98 aas. A suitable DRB4 β1 domain for inclusion in a CIIC polypeptide may comprise an aa sequence having at least 90% or at least 95% aa sequence identity to aas 1-88 of DRB4*01:03, and optionally having a length of about 95 aas, including, e.g., 89, 90, 91, 92, 93, 94, 95, 96, 97, or 98 aas.

A suitable DRB4 β2 domain for inclusion in a CIIC polypeptide may comprise an aa sequence having at least 85% (e.g., at least 90%, at least 95%, at least 98%) or 100% aa sequence identity to aas 96-188 of any of the DRB4 alleles provided in FIG. 7, and optionally having a length of about 93 aas, including, e.g., 87, 88, 89, 90, 91, 92, 93, 94, 95, or 96 aas. A suitable DRB4 β2 domain for inclusion in a CIIC polypeptide may comprise an aa sequence having at least 90% or at least 95% aa sequence identity to aas 96-188 of any of the DRB4 alleles provided in FIG. 7, and optionally having a length of about 93 aas, including, e.g., 87, 88, 89, 90, 91, 92, 93, 94, 95, or 96 aas. A suitable DRB4 β2 domain for inclusion in a CIIC polypeptide may comprise an aa sequence having at least 90% or at least 95% aa sequence identity to aas 96-188 of DRB4*01:01, and optionally having a length of about 93 aas, including, e.g., 87, 88, 89, 90, 91, 92, 93, 94, 95, or 96 aas. A suitable DRB4 β2 domain for inclusion in a CIIC polypeptide may comprise an aa sequence having at least 90% or at least 95% aa sequence identity to aas 96-188 of DRB4*01:03, and optionally having a length of about 93 aas, including, e.g., 87, 88, 89, 90, 91, 92, 93, 94, 95, or 96 aas.

### (d) DRB5 Polypeptides

A suitable MHC Class II DRB5 polypeptide for inclusion in a CIIC may have at least 85%, at least 90%, at least 95%, at least 98%, at least 99%, or 100% aa sequence identity with at least 165, at least 170, at least 175, at least 180, or at least 185 contiguous aas of the β1 and β2 domain region of a DRB5 aa sequence depicted in FIG. 8 or a naturally occurring allelic variant thereof. In some cases, the DRB5 polypeptide has a length of about 188 aas, including, e.g., 178, 179, 180, 181, 182, 183, 184, 185, 186, 187, or 188 aas. As used herein, the term “DRB5 polypeptide” includes allelic variants, e.g., naturally occurring allelic variants. Thus, in some cases, a suitable DRB5 polypeptide comprises a sequence that comprises aas 1-188 of DRB5*01:01 (see FIG. 8), or an allelic variant thereof.

A suitable DRB5 aa sequence for inclusion in a CIIC polypeptide may have at least 85%, at least 90%, at least 95%, at least 98%, at least 99%, or 100% aa sequence identity with at least 165, at least 170, at least 175, at least 180, or at least 185 contiguous aas of the β1 and β2 domain sequences of the DRB5*01:01 sequence depicted in FIG. 8. A suitable DRB5 aa sequence for inclusion in a CIIC polypeptide may have at least 90% or 100% aa sequence identity to at least 170 contiguous aas of the DRB5 β1 and β2 domain sequences of DRB5*01:01. A suitable DRB5 polypeptide may comprise an aa sequence having at least 95% or at least 98% aa sequence identity to at least 170 or at least 180 contiguous aas of the DRB5*01:01 β1 and β2 domain sequence aas 1 through 188 (see FIG. 8).

A CIIC may comprise a variant DRB5 polypeptide that comprises a non-naturally occurring Cys residue (e.g., for forming a body disulfide bond that stabilizes the CIIC). For example, a CIIC may comprise a variant DRB5 polypeptide comprising a Cys substitution for formation of a body disulfide bond at any one of aas 1-8 of the DRB5 sequences shown in FIG. 8. Alternatively, a CIIC may comprise a Cys substitution for formation of a body disulfide bond at any one of aas 5-8 of the DRB5 sequences shown in FIG. 8. Accordingly, a suitable DRB5 aa sequence for inclusion in a CIIC polypeptide may have at least 90% or at least 95% aa sequence identity to at least 170 contiguous aas of a DRB5 β1 and β2 domain sequence provided in FIG. 8, wherein the β1 sequence comprises a cysteine as a substitution for one of the aas in the subsequence PRFL (SEQ ID NO:194) (e.g., as a P5C or an F7C substitution). In one instance the DRB5 sequence is DRB5*01:01.

A suitable DRB5 β1 domain for inclusion in a CIIC polypeptide may comprise an aa sequence having at least 85% (e.g., at least 90%, at least 95%, at least 98%) or 100% aa sequence identity to aas 1-95 of the DRB5 alleles provided in FIG. 8, and optionally having a length of about 95 aas, including, e.g., 89, 90, 91, 92, 93, 94, 95, 96, 97, or 98 aas. A suitable DRB5 β1 domain for inclusion in a CIIC polypeptide may comprise an aa sequence having at least 90% or at least 95% aa sequence identity to aas 1-88 of DRB5*01:01, and optionally having a length of about 95 aas, including, e.g., 89, 90, 91, 92, 93, 94, 95, 96, 97, or 98 aas.

A suitable DRB5 β2 domain for inclusion in a CIIC polypeptide may comprise an aa sequence having at least 85% (e.g., at least 90%, at least 95%, at least 98%) or 100% aa sequence identity to aas 96-188 of the DRB5 alleles provided in FIG. 8, and optionally having a length of about 93 aas, including, e.g., 87, 88, 89, 90, 91, 92, 93, 94, 95, or 96 aas. A suitable DRB5 β2 domain for inclusion in a CIIC polypeptide may comprise an aa sequence having at least 90% or at least 95% aa sequence identity to aas 96-188 of DRB5*01:01, and optionally having a length of about 93 aas, including, e.g., 87, 88, 89, 90, 91, 92, 93, 94, 95, or 96 aas.

### (2) DPB Polypeptides

MHC Class II DPB polypeptides for inclusion in a CIIC have both β1 and β2 domains. Some non-limiting examples of DPB polypeptides are provided in FIG. 10. The β1 and β2 domains of the DPB proteins shown in those Figures are typically about 186 aas in length, with aas 1-92 making up the β1 domain and aas 93-186 making up the 12 domain. Aas 187-196 make up the membrane proximal region.

### (a) DPB1 Polypeptides

A suitable MHC Class II DPB1 polypeptide for inclusion in a CIIC may have at least 85%, at least 90%, at least 95%, at least 98%, at least 99%, or 100% aa sequence identity with at least 165, at least 170, at least 175, at least 180, or at least 185 contiguous aas of the β1 and β2 domain regions of a DPB1 aa sequence depicted in FIG. 10 or a naturally occurring allelic variant thereof. In some cases, the DPB1 polypeptide has a length of about 186 aas, including, e.g., 178, 179, 180, 181, 182, 183, 184, 185, 186, 187, or 188 aas. As used herein, the term “DPB1 polypeptide” includes allelic variants, e.g., naturally occurring allelic variants. Thus, in some cases, a suitable DPB1 polypeptide comprises a sequence that comprises aas 1-186 of DPB1*01:01 (see FIG. 10), or an allelic variant thereof. In some cases, the allelic variant is the DPB1*02:01 or DPB1*03:01 (see FIG. 10).

A suitable DPB1 aa sequence for inclusion in a CIIC polypeptide may have at least 85%, at least 90%, at least 95%, at least 98%, at least 99%, or 100% aa sequence identity with at least 165, at least 170, at least 175, at least 180, or at least 185 contiguous aas of the β1 and β2 domain sequences of the DPB1*01:01, DPB1*02:01, DPB1*03:01, or DPB1*11:01 sequences depicted in FIG. 10. A suitable DPB1 aa sequence for inclusion in a CIIC polypeptide may have at least 90% or 100% aa sequence identity to at least 170 contiguous aas of the DPB1 β1 and β2 domain sequence of DPB1*01:01, DPB1*02:01, DPB1*03:01, or DPB1*11:01. A suitable DPB1 aa sequence for inclusion in a CIIC polypeptide may have at least 95% or at least 98% aa sequence identity to at least 170 contiguous aas of the DPB1 β1 and β2 domain sequences of DPB1*01:01, DPB1*02:01, DPB1*03:01, or DPB1*11:01. Thus, a suitable DPB1 polypeptide may comprise an aa sequence having at least 95% or at least 98% aa sequence identity to at least 170 or at least 180 contiguous aas of the DPB1*01:01 β1 and β2 domain sequence aas 1 through 188 (see FIG. 10). A suitable DPB1 polypeptide may comprise an aa sequence having at least 95% aa or at least 98% sequence identity to at least 180 contiguous aas of the DPB1*02:01 β1 and β2 domain sequence aas 1 through 186 (see FIG. 10). A suitable DPB1 polypeptide may comprise an aa sequence having at least 95% or at least 98% aa sequence identity to at least 180 contiguous aas of the DPB1*03:01 β1 and β2 domain sequence aas 1 through 186 (see FIG. 10). A suitable DPB1 polypeptide may comprise an aa sequence having at least 95% aa or at least 98% sequence identity to at least 180 contiguous aas of the DPB1*11:01 β1 and β2 domain sequence aas 1 through 186 (see FIG. 10).

A CIIC may comprise a variant DPB1 polypeptide that comprises a non-naturally occurring Cys residue (e.g., for forming a body disulfide bond that stabilizes the CIIC). For example, a CIIC may comprise a variant DPB1 polypeptide comprising a Cys substitution for formation of a body disulfide bond at any one of aas 1-8 of the DPB1 sequences shown in FIG. 10. Alternatively, a CIIC may comprise a Cys substitution for formation of a body disulfide bond at any one of aas 4-8 or 5-8 of the DPB1 sequences shown in FIG. 10. Accordingly, a suitable DPB1 aa sequence for inclusion in a CIIC polypeptide may have at least 90% or at least 95% aa sequence identity to at least 170 contiguous aas of a DPB1 β1 and β2 domain sequence provided in FIG. 10, wherein the β1 sequence comprises a cysteine as a substitution for one of the aas in the subsequence PENYL (SEQ ID NO:195) or PENYV (SEQ ID NO:196) (e.g., a P4C, ESC, N6C or Y7C substitution). In one instance the DPB1 sequence is DPB1*01:01. In another instance, the DPB1 sequence is DPB1*02:01 or DPB1*03:01. In another instance, the DPB1 sequence is DPB1*11:01.

A suitable DPB1 β1 domain for inclusion in a CIIC polypeptide may comprise an aa sequence having at least 85% (e.g., at least 90%, at least 95%, at least 98%) or 100% aa sequence identity to aas 1-92 of any of the DPB1 alleles provided in FIG. 10, and optionally having a length of about 92 aas, including, e.g., 89, 90, 91, 92, 93, 94, 95, 96, 97, or 98 aas. A suitable DPB1 β1 domain for inclusion in a CIIC polypeptide may comprise an aa sequence having at least 90% or at least 95% aa sequence identity to aas 1-92 of any of the DPB1 alleles provided in FIG. 10, and optionally having a length of about 92 aas, including, e.g., 89, 90, 91, 92, 93, 94, 95, 96, 97, or 98 aas. A suitable DPB1 β1 domain for inclusion in a CIIC polypeptide may comprise an aa sequence having at least 90% or at least 95% aa sequence identity to aas 1-88 of DPB1*01:01, and optionally having a length of about 92 aas, including, e.g., 89, 90, 91, 92, 93, 94, 95, 96, 97, or 98 aas. A suitable DPB1 β1 domain for inclusion in a CIIC polypeptide may comprise an aa sequence having at least 90% or at least 95% aa sequence identity to aas 1-88 of DPB1*02:01 or DPB1*03:01, and optionally having a length of about 92 aas, including, e.g., 89, 90, 91, 92, 93, 94, 95, 96, 97, or 98 aas. A suitable DPB1 β1 domain for inclusion in a CIIC polypeptide may comprise an aa sequence having at least 90% or at least 95% aa sequence identity to aas 1-88 of DPB1*11:01, and optionally having a length of about 92 aas, including, e.g., 89, 90, 91, 92, 93, 94, 95, 96, 97, or 98 aas.

A suitable DPB1 β2 domain for inclusion in a CIIC polypeptide may comprise an aa sequence having at least 85% (e.g., at least 90%, at least 95%, at least 98%) or 100% aa sequence identity to aas 93-186 of any of the DPB1 alleles provided in FIG. 10, and optionally having a length of about 93 aas, including, e.g., 87, 88, 89, 90, 91, 92, 93, 94, 95, or 96 aas. A suitable DPB1 β2 domain for inclusion in a CIIC polypeptide may comprise an aa sequence having at least 90% or at least 95% aa sequence identity to aas 93-186 of any of the DPB1 alleles provided in FIG. 10, and optionally having a length of about 93 aas, including, e.g., 87, 88, 89, 90, 91, 92, 93, 94, 95, or 96 aas. A suitable DPB1 β2 domain for inclusion in a CIIC polypeptide may comprise an aa sequence having at least 90% or at least 95% aa sequence identity to aas 93-186 of DPB1*01:01, and optionally having a length of about 93 aas, including, e.g., 87, 88, 89, 90, 91, 92, 93, 94, 95, or 96 aas. A suitable DPB1 β2 domain for inclusion in a CIIC polypeptide may comprise an aa sequence having at least 90% or at least 95% aa sequence identity to aas 93-186 of DPB1*02:01 or DPB1*03:01, and optionally having a length of about 93 aas, including, e.g., 87, 88, 89, 90, 91, 92, 93, 94, 95, or 96 aas. A suitable DPB1 β2 domain for inclusion in a CIIC polypeptide may comprise an aa sequence having at least 90% or at least 95% aa sequence identity to aas 93-186 of DPB1*11:01, and optionally having a length of about 93 aas, including, e.g., 87, 88, 89, 90, 91, 92, 93, 94, 95, or 96 aas.

### (3) DQB Polypeptides

MHC Class II DQB polypeptides for inclusion in a CIIC have both β1 and β2 domains. Some non-limiting examples of DQB polypeptides are provided in FIGS. 13 and 14. The β1 and β2 domains of the DQB proteins shown in those Figures are typically about 187 or 188 aas in length, with aas 1-94 making up the β1 domain and aas 95-187 or 95-188 making up the β2 domain. Aas 188-197 or 189-198 make up the membrane proximal region.

### (a) DQB1 Polypeptides

A suitable MHC Class II DQB1 polypeptide for inclusion in a CIIC may have at least 85%, at least 90%, at least 95%, at least 98%, at least 99%, or 100% aa sequence identity with at least 165, at least 170, at least 175, at least 180, or at least 185 contiguous aas of the β1 and β2 domain regions of a DQB1 aa sequence depicted in FIG. 13 or a naturally occurring allelic variant thereof. In some cases, the DQB1 polypeptide has a length of about 188 aas, including, e.g., 178, 179, 180, 181, 182, 183, 184, 185, 186, 187, or 188 aas. As used herein, the term “DQB1 polypeptide” includes allelic variants, e.g., naturally occurring allelic variants. Thus, in some cases, a suitable DQB1 polypeptide comprises a sequence that comprises aas 1-188 of DQB1*02:01, DQB1*03:01, DQB1*04:01, DQB1*05:01, or DQB1*06:01 (see FIG. 13), or an allelic variant thereof. In some cases, the allelic variant is the DQB1*02:01, DQB1*02:02 or DQB1*03:01 (see FIG. 13).

A suitable DQB1 aa sequence for inclusion in a CIIC polypeptide may have at least 85%, at least 90%, at least 95%, at least 98%, at least 99%, or 100% aa sequence identity with at least 165, at least 170, at least 175, at least 180, or at least 185 contiguous aas of the β1 and β2 domain sequences of DQB1*02:01, DQB1*03:01, DQB1*04:01, DQB1*05:01, or DQB1*06:01 sequences depicted in FIG. 13. A suitable DQB1 aa sequence for inclusion in a CIIC polypeptide may have at least 90% or 100% aa sequence identity to at least 170 contiguous aas of the DQB1 β1 and β2 domain sequences of DQB1*02:01, DQB1*03:01, DQB1*04:01, DQB1*05:01, or DQB1*06:01. A suitable DQB1 aa sequence for inclusion in a CIIC polypeptide may have at least 95% or 100% aa sequence identity to at least 170 contiguous aas of the DQB1 β1 and β2 domain sequences of DQB1*02:01, DQB1*03:01, DQB1*04:01, DQB1*05:01, or DQB1*06:01. Thus, a suitable DQB1 polypeptide may comprise an aa sequence having at least 95% or at least 98% aa sequence identity to at least 170 or at least 180 contiguous aas of the DQB1*02:01, DQB1*03:01, DQB1*04:01, DQB1*05:01, or DQB1*06:01 β1 and β2 domain sequence aas 1 through 188 (see FIG. 13). A suitable DQB1 polypeptide may comprise an aa sequence having at least 95% or at least 98% aa sequence identity to at least 170 or at least 180 contiguous aas of the DQB1*02:01 β1 and β2 domain sequence aas 1 through 188. A suitable DQB1 polypeptide may comprise an aa sequence having at least 95% or at least 98% aa sequence identity to at least 170 or at least 180 contiguous aas of the DQB1*03:01 β1 and β2 domain sequence aas 1 through 188. A suitable DQB1 polypeptide may comprise an aa sequence having at least 95% or at least 98% aa sequence identity to at least 170 or at least 180 contiguous aas of the DQB1*04:01 or DQB1*05:01 β1 and β2 domain sequence aas 1 through 188. A suitable DQB1 polypeptide may comprise an aa sequence having at least 95% or at least 98% aa sequence identity to at least 170 or at least 180 contiguous aas of the DQB1*06:01 β1 and β2 domain sequence aas 1 through 188.

A CIIC may comprise a variant DQB1 polypeptide that comprises a non-naturally occurring Cys residue (e.g., for forming a body disulfide bond that stabilizes the CIIC). For example, a CIIC may comprise a variant DQB1 polypeptide comprising a Cys substitution for formation of a body disulfide bond at any one of aas 1-8 of the DQB1 sequences shown in FIG. 13. Alternatively, a CIIC may comprise a Cys substitution for formation of a body disulfide bond at any one of aas 4-8 or 5-8 of the DQB1 sequences shown in FIG. 13. Accordingly, a suitable DQB1 aa sequence for inclusion in a CIIC polypeptide may have at least 90% or at least 95% aa sequence identity to at least 170 contiguous aas of a DQB1 β1 and β2 domain sequence provided in FIG. 13, wherein the β1 sequence comprises a cysteine as a substitution for one of the aas in the subsequence PEDF (SEQ ID NO:197) (e.g., a P4C, ESC, D6C or F7C substitution). In one instance the DQB1 sequence is DQB1*02:01. In one instance the DQB1 sequence is DQB1*03:01. In another instance, the DQB1 sequence is DQB1*04:01 or DQB1*05:01. In another instance, the DQB1 sequence is DQB1*06:01.

A suitable DQB1 β1 domain for inclusion in a CIIC polypeptide may comprise an aa sequence having at least 85% (e.g., at least 90%, at least 95%, at least 98%) or 100% aa sequence identity to aas 1-94 of any of the DQB1 alleles provided in FIG. 13, and optionally having a length of about 94 aas, including, e.g., 89, 90, 91, 92, 93, 94, 95, 96, 97, or 98 aas. A suitable DQB1 β1 domain for inclusion in a CIIC polypeptide may comprise an aa sequence having at least 90% or at least 95% aa sequence identity to aas 1-94 of any of the DQB1 alleles provided in FIG. 13, and optionally having a length of about 94 aas, including, e.g., 89, 90, 91, 92, 93, 94, 95, 96, 97, or 98 aas. A suitable DQB1 β1 domain for inclusion in a CIIC polypeptide may comprise an aa sequence having at least 90% or at least 95% aa sequence identity to aas 1-88 of DQB1*02:01, and optionally having a length of about 94 aas, including, e.g., 89, 90, 91, 92, 93, 94, 95, 96, 97, or 98 aas. A suitable DQB1 β1 domain for inclusion in a CIIC polypeptide may comprise an aa sequence having at least 90% or at least 95% aa sequence identity to aas 1-88 of DQB1*03:01, and optionally having a length of about 94 aas, including, e.g., 89, 90, 91, 92, 93, 94, 95, 96, 97, or 98 aas. A suitable DQB1 β1 domain for inclusion in a CIIC polypeptide may comprise an aa sequence having at least 90% or at least 95% aa sequence identity to aas 1-88 of DQB1*04:01 or DQB1*05:01, and optionally having a length of about 94 aas, including, e.g., 89, 90, 91, 92, 93, 94, 95, 96, 97, or 98 aas. A suitable DQB1 β1 domain for inclusion in a CIIC polypeptide may comprise an aa sequence having at least 90% or at least 95% aa sequence identity to aas 1-88 of DQB1*06:01, and optionally having a length of about 94 aas, including, e.g., 89, 90, 91, 92, 93, 94, 95, 96, 97, or 98 aas.

A suitable DQB1 β2 domain for inclusion in a CIIC polypeptide may comprise an aa sequence having at least 85% (e.g., at least 90%, at least 95%, at least 98%) or 100% aa sequence identity to aas 95-188 of any of the DQB1 alleles provided in FIG. 13, and optionally having a length of about 94 aas, including, e.g., 87, 88, 89, 90, 91, 92, 93, 94, 95, or 96 aas. A suitable DQB1 β2 domain for inclusion in a CIIC polypeptide may comprise an aa sequence having at least 90% or at least 95% aa sequence identity to aas 95-188 of any of the DQB1 alleles provided in FIG. 13, and optionally having a length of about 94 aas, including, e.g., 87, 88, 89, 90, 91, 92, 93, 94, 95, or 96 aas. A suitable DQB1 β2 domain for inclusion in a CIIC polypeptide may comprise an aa sequence having at least 90% or at least 95% aa sequence identity to aas 95-188 of DQB1*02:01, and optionally having a length of about 94 aas, including, e.g., 87, 88, 89, 90, 91, 92, 93, 94, 95, or 96 aas. A suitable DQB1 β2 domain for inclusion in a CIIC polypeptide may comprise an aa sequence having at least 90% or at least 95% aa sequence identity to aas 95-188 of DQB1*03:01, and optionally having a length of about 94 aas, including, e.g., 87, 88, 89, 90, 91, 92, 93, 94, 95, or 96 aas. A suitable DQB1 β2 domain for inclusion in a CIIC polypeptide may comprise an aa sequence having at least 90% or at least 95% aa sequence identity to aas 95-188 of DQB1*04:01 or DQB1*05:01, and optionally having a length of about 94 aas, including, e.g., 87, 88, 89, 90, 91, 92, 93, 94, 95, or 96 aas. A suitable DQB1 β2 domain for inclusion in a CIIC polypeptide may comprise an aa sequence having at least 90% or at least 95% aa sequence identity to aas 95-188 of DQB1*06:01, and optionally having a length of about 94 aas, including, e.g., 87, 88, 89, 90, 91, 92, 93, 94, 95, or 96 aas.

### (b) DQB2 Polypeptides

A suitable MHC Class II DQB2 polypeptide for inclusion in a CIIC may have at least 85%, at least 90%, at least 95%, at least 98%, at least 99%, or 100% aa sequence identity with at least 165, at least 170, at least 175, at least 180, or at least 185 contiguous aas of the β1 and β2 domain regions of a DQB2 aa sequence depicted in FIG. 14 or a naturally occurring allelic variant thereof. In some cases, the DQB2 polypeptide has a length of about 187 aas, including, e.g., 178, 179, 180, 181, 182, 183, 184, 185, 186, 187, or 188 aas. As used herein, the term “DQB2 polypeptide” includes allelic variants, e.g., naturally occurring allelic variants. Thus, in some cases, a suitable DQB2 polypeptide comprises a sequence that comprises aas 1-187 of DQB2 isoform 1 (DQB2-Iso-1) or DQB2 isoform 2 (DQB2-Iso-2) (see FIG. 14), or an allelic variant thereof. In some cases, the allelic variant is the DQB2-Iso-1 or DQB2-Iso-2 (see FIG. 14).

A suitable DQB2 aa sequence for inclusion in a CIIC polypeptide may have at least 85%, at least 90%, at least 95%, at least 98%, at least 99%, or 100% aa sequence identity with at least 165, at least 170, at least 175, at least 180, or at least 185 contiguous aas of the β1 and β2 domain sequences of the DQB2-Iso-1 or DQB2-Iso-2 sequences depicted in FIG. 14. A suitable DQB2 aa sequence for inclusion in a CIIC polypeptide may have at least 90% or 100% aa sequence identity to at least 170 contiguous aas of the DQB2 β1 and β2 domain sequences of DQB2-Iso-1 or DQB2-Iso-2. A suitable DQB2 aa sequence for inclusion in a CIIC polypeptide may have at least 95% or at least 98% aa sequence identity to at least 170 contiguous aas of the DQB2 β1 and β2 domain sequences of DQB2-Iso-1 or DQB2-Iso-2. Thus, a suitable DQB2 polypeptide may comprise an aa sequence having at least 95% or at least 98% aa sequence identity to at least 180 contiguous aas of the DQB2-Iso-1 or DQB2-Iso-2 β1 and β2 domain sequence aas 1 through 187 (see FIG. 14). A suitable DQB2 polypeptide may comprise an aa sequence having at least 95% aa sequence identity to at least 170 or at least 180 contiguous aas of the DQB2-Iso-1 β1 and β2 domain sequence aas 1 through 187. A suitable DQB2 polypeptide may comprise an aa sequence having at least 95% aa sequence identity to at least 170 or at least 180 contiguous aas of the DQB2-Iso-2 β1 and β2 domain sequence aas 1 through 187.

A CIIC may comprise a variant DQB2 polypeptide that comprises a non-naturally occurring Cys residue (e.g., for forming a body disulfide bond that stabilizes the CIIC). For example, a CIIC may comprise a variant DQB2 polypeptide comprising a Cys substitution for formation of a body disulfide bond at any one of aas 1-8 of the DQB2 sequences shown in FIG. 14. Alternatively, a CIIC may comprise a Cys substitution for formation of a body disulfide bond at any one of aas 4-8 or 5-8 of the DQB2 sequences shown in FIG. 14. Accordingly, a suitable DQB2 aa sequence for inclusion in a CIIC polypeptide may have at least 90% or at least 95% aa sequence identity to at least 170 contiguous aas of a DQB2-Iso-1 or DQB2-Iso-2 β1 and β2 domain sequence provided in FIG. 14, wherein the β1 sequence comprises a cysteine substitution in the subsequence PKDFL (SEQ ID NO:198) (e.g., a P4C, K5C, D6C or F7C substitution). In one instance the DQB2 sequence is DQB2-Iso-1. In one instance the DQB2 sequence is DQB2*03:01. In another instance, the DQB2 sequence is DQB2-Iso-2.

A suitable DQB2 β1 domain for inclusion in a CIIC polypeptide may comprise an aa sequence having at least 85% (e.g., at least 90%, at least 95%, at least 98%) or 100% aa sequence identity to aas 1-94 of any of the DQB2 alleles provided in FIG. 14, and optionally having a length of about 94 aas, including, e.g., 89, 90, 91, 92, 93, 94, 95, 96, 97, or 98 aas. A suitable DQB2 β1 domain for inclusion in a CIIC polypeptide may comprise an aa sequence having at least 90% or at least 95% aa sequence identity to aas 1-94 of any of the DQB2 alleles (DQB2-Iso-1 or DQB2-Iso-2) provided in FIG. 14, and optionally having a length of about 94 aas, including, e.g., 89, 90, 91, 92, 93, 94, 95, 96, 97, or 98 aas.

A suitable DQB2 β2 domain for inclusion in a CIIC polypeptide may comprise an aa sequence having at least 85% (e.g., at least 90%, at least 95%, at least 98%) or 100% aa sequence identity to aas 95-187 of any of the DQB2 alleles provided in FIG. 14, and optionally having a length of about 93 aas, including, e.g., 87, 88, 89, 90, 91, 92, 93, 94, 95, or 96 aas. A suitable DQB2 β2 domain for inclusion in a CIIC polypeptide may comprise an aa sequence having at least 90% or at least 95% aa sequence identity to aas 95-187 of any of the DQB2 alleles (DQB2-Iso-1 or DQB2-Iso-2) provided in FIG. 14, and optionally having a length of about 93 aas, including, e.g., 87, 88, 89, 90, 91, 92, 93, 94, 95, or 96 aas.

### c) Membrane Proximal Regions

In addition to the α1, α2, β1, and β2 domain sequences of the Class II MHC (HLA) subunits present in CIICs, the CIICs may comprise membrane proximal regions. For example, in addition to the MHC 1 subunit β1 and β2 domain sequences, the MHC 1 subunit sequences may comprise a membrane proximal region (e.g., an MHC β subunit membrane proximal region). Similarly, in addition to the MHC α subunit α1 and α2 domain sequences, the MHC α subunit sequences may comprise a membrane proximal region (e.g., an MHC α subunit membrane proximal region). The membrane proximal regions are typically located following (are located on the C-terminal side) the β2 and/or α2 domain sequences. A membrane proximal region following an MHC 1 subunit sequence may have at least 85%, at least 90%, or at least 95% aa sequence identity to the membrane proximal region associated with the MHC β subunit present in the CIIC (e.g., 1 or 2 aa substitutions).

The membrane proximal region following the β2 domain sequence (see FIG. 1) may be from the same allele as the β2 domain sequence present in the CIIC, and may be located so that the sequence from the N-terminus of the β2 domain to the C-terminus of the membrane proximal region corresponds to the sequence of the allele from which they were derived. For example, where a CIIC comprises an HLA DQB1*02:01 β2 domain, the membrane proximal region of the DQB1*02:01 allele may directly follow the β2 domain sequence.

Similar to the situation with MHC 1 subunit sequences, the membrane proximal region following an MHC α subunit sequence may have at least 85%, at least 90%, or at least 95% aa sequence identity to the membrane proximal region associated with an MHC α subunit (e.g., 1 or 2 aa substitutions). The membrane proximal region may be from the same allele as the α2 domain sequence present in the CIIC, and may be located so that the sequence from the N-terminus of the α2 domain to the C-terminus of the membrane proximal region corresponds to the sequence of the allele from which they were derived. For example, where a CIIC comprises an HLA DQA1*05:01 α2 domain, the membrane proximal region of the DQA1*05:01 allele may directly follow the α2 domain sequence.

### d) MHC Class II Disease Risk-Associated Alleles and Haplotypes

Certain alleles and haplotypes of MHC Class II have been associated with disease, e.g., increased risk of developing a particular disease. See, e.g., Erlich et al. (2008) Diabetes 57:1084; Gough and Simmonds (2007) Curr. Genomics 8:453; Mitchell et al. (2007) Robbins Basic Pathology Philadelphia: Saunders, 8^{th} ed.; Margaritte-Jeannin et al. (2004) Tissue Antigens 63:562; and Kurko et al. (2013) Clin. Rev. Allergy Immunol. 45:170. A number of those diseases and their associated alleles and/or haplotypes are described in WO 2020/181273 assigned to Cue Biopharma, Inc., and references cited therein. Some HLA haplotypes and alleles associated with increased risk that an individual expressing such HLA haplotypes and/or alleles will develop a given autoimmune disease are set forth in the table provided in FIG. 17. That table also provides a listing of some molecules associated with the disease (e.g., autoantigens such as proteins and peptides) that can function as epitopes or a source of epitopes. Some HLA haplotypes and alleles associated with increased risk that an individual expressing such HLA haplotypes and/or alleles will develop Type 1 Diabetes are set forth in FIG. 17. A CIIC of the present disclosure that is directed to the treatment of a specific disease can include any of the disease associated HLA haplotypes and/or alleles and the corresponding epitopes set out in FIG. 17 or in FIG. 17. The peptide epitope can be, for example, a peptide of from 4 aas to about 25 aas in length of any of the autoantigens set out in FIG. 17 or FIG. 17.

The following are notes to the table provided in FIG. 17: 1) AH8.1 (e.g., HLA Δ1-B8-DR3-DQ2 haplotype); 2) DQ3 alleles include DQB1*03 alleles such as DQB1*03:01 to DQB1*03:05 proteins; 3) DQ5 alleles include DQB1*05 alleles such as DQB1*05:01 to DQB1*05:04 and may be associated with DQA1*01:01; 4) DR2 alleles include DRB1*15:01-15:04 and DRB1*16:01-16:06; 5) DR3 haplotypes include: DRB1*03:01, DRB1*03:02, DRB1*03:03, and DRB1*03:04; 6) DR4 haplotypes include: DRB1*04:01 through DRB1*04:13; AH=ancestral haplotype; 7) Simmonds et al., Am. J. Hum. Genet. 76:157-163, (2005), see the table in FIG. 17, HLAs with odds ratios greater than 1.5 include the following DRB1, DQB1 and DQA1 alleles: DRB1*03:01 to −03:05, −10:01, −08:01 to −11, −16:01 to −16:06, −11:01 to −11:21, −01:01 to −01:04, −04:01 to −04:22, and −15:01 to −15:05; DQB1*-02, −04, −03:01, −03:04, −05, −06:01 to −06:09, and −03:02; and HLA-DQA1*−05:01 to −05:02,−06:01,−04:01,−01:01,−01:02,−01:04,−01:03,−03:11, and−03:12; 8) Li et al., Mol Med Rep., 17(5): 6533-6541 (2018) noting epitopes from autoantigens including: SMD1 (NCBI Accession: CAE11897.1); SMD2 (NCBI Accession: AAC13776.1); SMD3 (NCBI Accession: AAA57034.1); Proliferating cell nuclear antigen (PCNA) (NCBI Accession: NP_872590.1); Acidic ribosomal phosphoprotein (P1) (NCBI Accession: AAA36471.1); Acidic ribosomal phosphoprotein (P2) (NCBI Accession: AAA36472.1); snRNP-B/B′ (NCBI Accession: P14678.2); U1-snRNP-C(NCBI Accession: NP_003084.1); U1-snRNP-A (NCBI Accession: NP_004587.1); Nucleolin (NCBI Accession: AAA59954.1); Acidic ribosomal phosphoprotein (PO) (NCBI Accession: AAA36470.1); DNA topoisomerase1 (truncated) (Unprot P11387); DNA topoisomerase 1 (full length) (NCBI Accession: NP_003277.1 and P11387); and U1-SnRNP 68/70 KDa (NCBI Accession: P08621.2).

### (1) Individual Disease Risk-Associated Alleles

The association of a number of HLA alleles with one or more autoimmune diseases is described in, for example, FIG. 17 and in Table 1. The sequences of the disease-associated alleles are provided in the figures accompanying this disclosure (e.g., DRB1 alleles are provided in the figures). Where disease associations are made to groups of alleles (e.g., DRB1*03), the sequences of additional alleles may be obtained from standard references including those provided by the U.S. National Center for Biotechnology Information (NCBI) or online at hla.alleles.org/nomenclature/index.html.

An exemplary association between various disease states and particular HLA alleles include the association of the alleles of the HLA-DR3 with early-age onset myasthenia gravis, Hashimoto's thyroiditis, autoimmune hepatitis, primary Sjögren's syndrome, and SLE. Other exemplary associations include: DRB1*0301 (“DRB1*03:01” provided in the figures) associationwith an increased risk of developing early onset Grave's disease and/or type 1 autoimmune hepatitis; DRB1*04:01 association with an increased risk of developing multiple sclerosis and/or rheumatoid arthritis; DRB1*04:02 association with increased risk of developing idiopathic pemphigus vulgaris, and/or SLE (e.g., SLE-associated anti-cardiolipin, SLE-associated anti-β2 glycoprotein I); DRB1*0403 association with increased risk of developing SLE (e.g., increased risk of developing SLE-associated anti-cardiolipin antibodies and/or SLE-associated anti-β2 glycoprotein I antibodies); DRB1*04:05 association with increased risk of developing rheumatoid arthritis and/or autoimmune hepatitis; and DRB1*04:06 association with increased risk of developing anti-caspase-8 autoantibodies (e.g., in silicosis-systemic sclerosis (SSc)-systemic lupus erythematosus (SLE)).

Certain DQB1 alleles are also associated with increased risk that an individual expressing such an allele will develop an autoimmune disease. For example, DQB1*0301, and DQB1*0602 are associated with an increased risk of developing MS and/or a more severe MS phenotype (e.g., more severe inflammatory and neurodegenerative damage).

### (a) MHC Class II Polypeptides in T1D

Alleles/isoforms showing increased association with T1D represent suitable sources of MHC Class II α1, a2, 11, and 12 polypeptide sequences for incorporation into CIICs directed to the treatment of T1D. T1D is associated with alleles belonging to the HLA-DR3 and HLA-DR4 haplotypes/serotypes, with the strongest risk associated with the HLA-DQ8, (e.g., HLA-DQB1*03:02) and alleles of the HLA-DQ2 serotype. Some high and moderate risk haplotypes and their association with various DR serotypes are shown in Table 1 adopted from Kantárová and Buc, Physiol. Res. 56: 255-266 (2007).

TABLE 1

| | | | | |
|---|---|---|---|---|
| DR serotype | DRB allele | DQ serotype | DQA allele | DQB allele |

High Risk T1D Haplotypes

| | | | | |
|---|---|---|---|---|
| DR3 | DRB1*0301 | DQ 2.5 | DQA1*0501 | DQB1*0201 |
| DR4 | DRB1*0401 | DQ 8.1 | DQA1*0301 | DQB1*0302 |
| DR4 | DRB1*0402 | DQ 8.1 | | |
| DR4 | DRB1*0405 | DQ 8.1 | | |

Moderate risk T1D haplotypes

| | | | | |
|---|---|---|---|---|
| DR1 | DRB1*01 | DQ 5 | DQA1*0101 | DQB1*0501 |
| DR8 | DRB1*0801 | | DQA1*0401 | DQB1*0402 |
| DR9 | DRB1*0901 | | DQA1*0301 | DQB1*0303 |

The stereotypically defined DR3 and DR4 protein isoforms/haplotypes of the DRB1 gene are associated with increased risk that an individual expressing such alleles will develop T1D. The DR3 serotype includes the alleles encoding the DRB1*03:01, *03:02, *03:03, and *03:04 proteins, with the HLA-DRB1*0301 allele often found associated with a predisposition to T1D. The DR4 serotype includes the alleles encoding the DRB1*04:01, *04:02, *04:03, *04:04, *04:05, *04:06, *04:07, *04:08, *04:09, *04:10, *04:11, *04:12, and *04:13 proteins. Certain HLA-DR4 proteins (e.g., HLA-DRB1*0401 and HLA-DRB1*0405) predispose individuals to T1D, whereas HLA-DRB1*04:03 allele/isoform may afford protection. DRB1*16:01 also shows an increased frequency in diabetic children relative to healthy controls (Deja, et al., Mediators of Inflammation 2006:1-7 (2006)). Alleles/isoforms showing increased association with T1D represent suitable sources of MHC II α1, α2, β1, and β2 polypeptide sequences.

DQ2 and DQ8 are serotypes within the HLA-DQ system that are determined by recognition of DQ β-chains. While T1D is associated with DR3 and DR4 alleles as discussed above, among the strongest associated risk factors for T1D are the presence of the HLA-DQ8 serotype (e.g., the HLA-DQB1*03:02 isoform), particularly the HLA-DQ8.1 serotype (HLA-DQA1*03:01/DQB1*03:02) and the alleles of the HLA-DQ2 serotype (e.g., DQB1*02 alleles such as DQB1*02:01, DQB1*02:02, or DQB1*02:03). Jones et al., Nat. Rev. Immunol. 2006, 6: 271-282. By contrast, individuals that carry the HLADQB1*0602 allele appear to be protected against type 1 diabetes. Id.

DQ2 is most common in Western Europe, North Africa, and East Africa, with the highest frequencies observed in parts of Spain and Ireland. Although the HLA-DR associations with T1D are not as strong as those of HLA-DQ, insulin-reactive T cells derived from lymph nodes draining the pancreas of patients with T1D appear to be HLA-DR4.1 restricted rather than HLA-DQ8 or HLA-DQ2 restricted (Kent et al., Nature 2005 435: 224-228). The crystal structure of HLA-DQ2 shows a distinctive P6 pocket with a large volume and polar character defined by the presence of Ser30β (see, e.g., FIG. 12 Ser 30) rather than Tyr30β, which is typically found in other HLA-DQ molecules. This is a unique feature of HLA-DQ2, as is the presence of a positively charged lysine residue at 711p (see FIG. 12 Lys 71) and, when combined with the polar nature of the P4 and P9 pockets, makes this MHC class II peptide binding groove the most suitable for accommodating peptides with negatively charged anchor residues (see, e.g., Jones et al, Nat. Rev. Immunol. 2006, 6: 271-282). This is a key factor in allowing HLA-DQ2 to present gluten-derived peptides that are high in proline and glutamate residues (generated by deamidation of glutamines). Id. In an embodiment, Ser30 of DQ2 (e.g., DQB1*02:01) molecules can be replaced with a cysteine (S30C) to permit conjugation of a peptide epitope that is co-translated as part of a T-cell modulatory antigen-presenting polypeptide to that position (e.g., utilizing a cysteine at position 6 of the peptide epitope).

The DQB1 locus alone has also been reported to be associated with T1D when position β57 is a neutral residue such as Ala or Ser. Both the DQ2 and DQ8 serotypes, which are associated with TID, lack an Asp at the 57p position, and instead have an Ala in its place (see, e.g., Ala 57 in FIG. 13, HLA-DQB1*02:01, and FIG. 19C, HLA-DQB1*03:02, respectively), leading to conferred T1D susceptibility. In contrast, DQB1*06:02, which has an Asp at position β57 (position 57 in FIG. 13), was found to be associated with resistance to T1D. Jones et al, Nat. Rev. Immunol. 2006, 6: 271-282. Position α57 of the molecule is a critical residue in the (P9) residue binding pocket of DQB1, which is involved in antigen presentation and T cell receptor (TCR) interaction.

Individuals with the HLA haplotype DQA1*03:01-DRB1*03:02, especially when combined with DQA1*05:01-DRB1*02:01, are highly susceptible (10-20-fold increase) to T1D, see Notkins, A. L., J. Biol. Chem., 2002, 277(46): 43545-48. Among the stereotypically defined groups showing susceptibility to T1D are HLA-DR4.1 (HLA-DRA1*01:01/DRB1*04:01), HLA-DR4.5 (HLA-DRA1*01:01/DRB1*04:05), HLA-DQ2.5 (HLA-DQA1*05:01/DQB1*02:01), and HLA-DQ8.1 (HLA-DQA1*03:01/DQB1*03:02). (see, e.g., Jones et al., Nat. Rev. Immunol. 2006, 6: 271-282). The DRB1*04:05-DQB1*04:01/DRB1*08:02-DQB1*03:02 genotype has shown to be associated with acute-onset and slow progressive T1D. Fulminant diabetes has been associated with DRB1*04:05-DQB1*040:1/DRB1*04:05-DQB1*04:01 genotype, in a Japanese population study (Kawabata, et al., Diabetologia 2009, 52:2513-21).

The above-mentioned alleles associated with an increased risk of T1D represent suitable candidates from which the α1, α2, β1, and/or β2 polypeptide sequences present in a CIIC may be taken. In an embodiment, the CIIC is DQ2.5-like with the α1 and α2 polypeptides from DQA1*0501, and the β1 and β2 polypeptides taken from DQB1*0201. In an embodiment, the CIIC is DQ8.1-like with the α1 and α2 polypeptides from DQA1*0301, and the β1 and β2 polypeptides taken from DQB1*0302.

The Table in FIG. 25 shows examples of HLA Class II α lleles, MODs, and T1D-epitopes that may be incorporated into a CIIC for T1D therapy.

The above-mentioned alleles associated with an increased risk of T1D represent suitable candidates from which the α1, α2, β1, and/or β2 polypeptide sequences present in a CIIC may be taken.

### (b) MHC Class II Polypeptides and Celiac Disease

HLA haplotypes DQ2 and DQ8 are associated with increased risk that an individual expressing such HLA haplotypes will develop celiac disease. DQ2 represents the second highest risk factor for celiac disease, the highest risk factor is a close family member with the disease. It is estimated that approximately 95% of all celiac patients have at least one DQ2 allele, and of those individuals about 30% have two copies of a DQ2 allele. DQ2 isoforms vary in their association with celiac disease, the DQ2.5 isoform (DQB1*02:01/DQA1*05:01) being strongly associated. DQB1*0201 is genetically linked to DQA1*05:01 forming the DQ2.5 haplotype. DQ2.5 is present in high levels in northern, islandic Europe, and the Basque region of Spain with the phenotype frequency exceeding 50% in 0parts of Ireland.

The immunodominant site for DQ2.5 is on α2-gliadin, which has a protease resistant 33mer that has 6 overlapping DQ2.5 restricted epitopes. The multiple epitopes produce strong binding of T-cells to the DQ2.5-33mer complexes. DQ2.5 binds gliadin, but the binding is sensitive to deamidation caused by tissue transglutaminase, whose action produces most of the highest affinity sites/epitopes. All or part of the 33mer (LQLQPFPQPELPYPQPELPYPQPELPYPQPQPF, SEQ ID NO:200) or a similarly described 19mer (LGQQQPFPPQQPYPQPQPF, SEQ ID NO:201) (e.g., 8 or more, 9, or more, 10 or more, 12, or more, 14 or more, or 16 or more contiguous amino acids) may be utilized as a peptide epitope. See, e.g., Bruun et al. 2016, J. Diabetes Res. 2016, 2016:1-11 Article ID 2424306.

As noted above, T1D is associated with the DQ2.5 phenotype, and there may be a link between Gluten-Sensitive Enteropathy (GSE) and early onset male T1D. Recent studies indicate a combination of DQ2.5 and DQ8 (both acid peptide presenters) greatly increase the risk of adult onset T1D. The presence of DQ2 with DR3 may decrease the age of onset and the severity of the disorders.

While the DQ2.5 haplotype confers the single highest known genetic risk for celiac disease, comparable risk can also come from very similar alleles of different haplotypes (e.g., other DQA1*05 and DQB1*02 alleles). The DQ2.2 phenotype has the form α2-β2 (e.g., DQA1*02:01:DQB1*02:02) and is associated with the occurrence of some celiac disease. Because the HLA DQB1*02:02 and its linked DQA1* alleles of the DQ2.2 haplotype do not produce a DQA1*05 subunit (α5 e.g., DQA1*05:01), the heterodimer cannot effectively present α-2 gliadin; it can, however, present other gliadins. Accordingly, a multimeric or single chain T-cell modulatory antigen-presenting polypeptide comprising DQ 2.2 polypeptide sequences (e.g., DQA1*02:01:DQB1*02:02) may be used to present non-α-2 gliadin peptides.

The DQ2.2/DQ7.5 phenotype, also referred to as DQ2.5trans, is also associated with celiac disease. The serotypically defined DQ7.5 phenotype has a DQA1*0505:DQB1*0301 haplotype. When DQA1*0505 or DQA1*0501 gene products are processed to the cell surface they become the α5 and can assemble an MHC class II molecule with either of the DQ 2.2 alleles, DQB1*0202 and DQB1*0201. As a result, the isoforms produced by the phenotype of two haplotypes, DQ2.2/DQ7.5, include HLA DQ α⁵β²(DQ2.5), α²β²(DQ2.2), α²β⁷(DQ7.2, e.g., DQA1*0201:DQB1*0301), and α⁵β⁷(DQ7.5).

DQ8 is typically involved in celiac disease in those individuals where DQ2 is not present. The DQ8.1 haplotype encodes the DQA1*0301:DQB1*0302 haplotype. DQ8 is extremely high in Native Americans of Central America and tribes of Eastern American origin.

Two Class II HLA genotypes (DQA1*05:DQB1*02 {α⁵β²} and DQA1*03:DQB1* 03:02 {an α³β³}) contribute substantially to the genetic risk of celiac disease in families, and have been suggested to be virtually required for celiac disease to occur in Caucasian individuals (see Murry et al., Clin. Gastroenterol. Hepatol. 2007, 5(12): 1406-1412). Among the stereotypically defined groups showing susceptibility to T1D and Celiac disease are HLA-DQ2.5 (HLA-DQA1*05:01/DQB1*02:01) and HLA-DQ8.1 (HLA-DQA1*03:01/DQB1*03:02) (see, e.g., Jones et al., Nat. Rev. Immunol. 2006, 6: 271-282).

The alleles associated with an increased risk of celiac disease described above represent suitable candidates from which the α1, α2, β1, and/or β2 polypeptide sequences of CIICs may be taken. In an embodiment, the CIIC is DQ2.5-like with the α1 and α2 polypeptides from DQA1*0501, and the β1 and β2 polypeptides taken from DQB1*0201. In an embodiment, the CIIC is DQ2.2-like with the α1 and α2 polypeptides from DQA1*02:01, and the β1 and β2 polypeptides taken from DQB1*02:01. In an embodiment, the CIIC is DQ8.1-like with the α1 and α2 polypeptides from DQA1*0301, and the β1 and β2 polypeptides taken from DQB1*0302. In an embodiment, the CIIC comprises α1, α2, 11, and 12 polypeptides taken from isoforms produced by the DQ2.2/DQ7.5 haplotypes, including the HLA DQ α⁵β²(DQ2.5), α²β²(DQ2.2), α²β⁷(DQ7.2, e.g., DQA1*0201:DQB1*0301), and α⁵β⁷(DQ7.5) molecules.

### DRB1*03:01

DRB1*0301 (“DRB1*03:01” in FIG. 5) is associated with an increased risk of developing T1D. Thus, a CIIC may comprise a DRB1*03:01 polypeptide comprising an aa sequence having at least 90%, at least 95%, at least 98% or 100% aa sequence identity to the β1 and β2 domains (aas 1-188) of the DRB1*03:01 aa sequence depicted in FIG. 5. A CIIC may comprise a DRB1*03:01 polypeptide comprising an aa sequence having at least 90% or at least 95% aa sequence identity to the β1 domain of the DRB1*03:01 aa sequence depicted in FIG. 5. A CIIC may comprise a DRB1*03:01 polypeptide comprising an aa sequence having at least 90% or at least 95% aa sequence identity to the β2 domain of the DRB1*03:01 aa sequence depicted in FIG. 5.

### DRB1*04:01

DRB1*0401 (“DRB1*04:01” in FIG. 5) is associated with increased risk of developing T1D. Thus, a CIIC may comprise a DRB1*04:01 polypeptide comprising an aa sequence having at least 85%, at least 90%, at least 95%, at least 98%, at least 99%, or 100% aa sequence identity to the β1 and β2 domains (aas 1-188) of the DRB1*04:01 aa sequence depicted in FIG. 5. A CIIC may comprise a DRB1*04:01 polypeptide comprising an aa sequence having at least 90% or at least 95% aa sequence identity to the β1 domain of the DRB1*04:01 aa sequence depicted in FIG. 5. A CIIC may comprise a DRB1*04:01 polypeptide comprising an aa sequence having at least 90% or at least 95% aa sequence identity to the β2 domain of the DRB1*04:01 aa sequence depicted in FIG. 5.

DRB1*04:02

DRB1*0402 (“DRB1*04:02” in FIG. 5) is associated with increased risk of developing T1D. Thus, a CIIC may comprise a DRB1*04:02 polypeptide comprising an aa sequence having at least 85%, at least 90%, at least 95%, at least 98%, at least 99%, or 100% aa sequence identity to the β1 and β2 domains (aas 1-188) of the DRB1*04:02 aa sequence depicted in FIG. 5. A CIIC may comprise a DRB1*04:02 polypeptide comprising an aa sequence having at least 90% or at least 95% aa sequence identity to the β1 domain of the DRB1*04:02 aa sequence depicted in FIG. 5. A CIIC may comprise a DRB1*04:02 polypeptide comprising an aa sequence having at least 90% or at least 95% aa sequence identity to the β2 domain of the DRB1*04:02 aa sequence depicted in FIG. 5.

### DRB1*04:05

DRB1*0405 (“DRB1*04:05” in FIG. 5) is associated with increased risk of developing T1D. Thus, a CIIC may comprise a DRB1*04:05 polypeptide comprising an aa sequence having at least 85%, at least 90%, at least 95%, at least 98%, at least 99%, or 100% aa sequence identity to the β1 and β2 domains (aas 1-188) of the DRB1*04:05 aa sequence depicted in FIG. 5. A CIIC may comprise a DRB1*04:05 polypeptide comprising an aa sequence having at least 90% or at least 95% aa sequence identity to the β1 domain of the DRB1*04:05 aa sequence depicted in FIG. 5. A CIIC may comprise a DRB1*04:05 polypeptide comprising an aa sequence having at least 90% or at least 95% aa sequence identity to the β2 domain of the DRB1*04:05 aa sequence depicted in FIG. 5.

### DQA1*05:01-DQB1*02:01 (DQ2.5)

DQ2.5 (DQA1*05:01-DQB1*02:01) is associated with increased risk of developing celiac disease. Thus, a CIIC may comprise a DQA1*05:01 polypeptide comprising an aa sequence having at least 85%, at least 90%, at least 95%, at least 98%, at least 99%, or 100% aa sequence identity to the α1 and α2 domains (aas 1-181) of the DQA1*05:01 aa sequence depicted in FIG. 5. A CIIC may comprise a DQA1*05:01 polypeptide comprising an aa sequence having at least 90% or at least 95% aa sequence identity to the α1 domain of the DQA1*05:01 aa sequence depicted in FIG. 5. A CIIC may comprise a DQA1*05:01 polypeptide comprising an aa sequence having at least 90% or at least 95% aa sequence identity to the α2 domain of the DQA1*05:01 aa sequence depicted in FIG. 5.

A CIIC may comprise a DQB1*02:01 polypeptide comprising an aa sequence having at least 85%, at least 90%, at least 95%, at least 98%, at least 99%, or 100% aa sequence identity to the β1 and β2 domains (aas 1-188) of the DQB1*02:01 aa sequence set forth in FIG. 13. A CIIC may comprise a DQB1*02:01 polypeptide comprising an aa sequence having at least 90% or at least 95% aa sequence identity to the β1 domain of the DQB1*02:01 aa sequence set forth in FIG. 13. A CIIC may comprise a DQB1*02:01 polypeptide comprising an aa sequence having at least 90% or at least 95% aa sequence identity to the β2 domain of the DQB1*02:01 aa sequence set forth in FIG. 13.

### DQA1*03:01-DQB1*03:02 (DQ8)

DQA1*03:01-DQB1*03:02 (DQ8) is associated with increased risk of developing celiac disease. Thus, a CIIC may comprise a DQA1*03:01 polypeptide comprising an aa sequence having at least 85%, at least 90%, at least 95%, at least 98%, at least 99%, or 100% aa sequence identity to the α1 and α2 domains (aas 1-181) of the DQA1*03:01 aa sequence depicted in FIG. 11. A CIIC may comprise a DQA1*03:01 polypeptide comprising an aa sequence having at least 90% or at least 95% aa sequence identity to the α1 domain of the DQA1*03:01 aa sequence depicted in FIG. 11. A CIIC may comprise a DQA1*03:01 polypeptide comprising an aa sequence having at least 90% or at least 95% aa sequence identity to the α2 domain of the DQA1*03:01 aa sequence depicted in FIG. 11.

A CIIC may comprise a DQB1*03:02 polypeptide comprising an aa sequence having at least 85%, at least 90%, at least 95%, at least 98%, at least 99%, or 100% aa sequence identity to the β1 and β2 domains (aas 1-188) of the DQB1:03:02 aa sequence set forth in FIG. 13. A CIIC may comprise a DQB1*03:02 polypeptide comprising an aa sequence having at least 90% or at least 95% aa sequence identity to the β1 domain of the DQB1*03:02 aa sequence set forth in FIG. 13. A CIIC may comprise a DQB1*03:02 polypeptide comprising an aa sequence having at least 90% or at least 95% aa sequence identity to the β2 domain of the DQB1*03:02 aa sequence set forth in FIG. 13.

### DRB1*04:01 and DRA1*01:01

A CIIC may comprise: i) an MHC α chain polypeptide comprising an aa sequence having at least 85%, at least 90%, at least 95%, at least 98%, at least 99%, or 100% aa sequence identity to the α1 and α2 domains (aas 1-181) of the DRA1*01:01 aa sequence provided in FIG. 4, and ii) an MHC β chain polypeptide comprising an aa sequence having at least 90% or at least 95% aa sequence identity to the β1 and β2 domains (aas 1-188) of the DRB1*04:01 aa depicted in FIG. 5. A CIIC may comprise: i) a DRA1*01:01 α chain polypeptide, and ii) a DRB1*04:01 β chain polypeptide.

### DQA1*05:01 and DQB1*02:01

A CIIC may comprise: i) a DQA1*05:01 α chain polypeptide, and ii) a DQB1*02:01 β chain polypeptide. A CIIC may comprise: i) an MHC α chain polypeptide comprising an aa sequence having at least 85%, at least 90%, at least 95%, at least 98%, at least 99%, or 100% aa sequence identity to the α1 and α2 domains (aas 1-181) of the DQA1*05:01 sequence depicted in FIG. 11, and ii) an MHC β chain polypeptide comprising an aa sequence having at least 85%, at least 90%, at least 95%, at least 98%, at least 99%, or 100% aa sequence identity to the β1 and β2 domains (aas 1-188) of the DQB1*02:01 sequence depicted in FIG. 13. A CIIC may comprise: i) an MHC α chain polypeptide comprising an aa sequence having at least 95% or at least 98% aa sequence identity to the α1 and α2 domains (aas 1-181) of the DQA1*05:01 sequence depicted in FIG. , 11 and ii) an MHC β chain polypeptide comprising an aa sequence having at least 95% or at least 98% aa sequence identity to the β1 and β2 domains (aas 1-188) of the DQB1*02:01 sequence depicted in FIG. 13.

### DQA1*03:01 and DQB1*03:02

A CIIC may comprise: i) an MHC α chain polypeptide comprising an aa sequence having at least 90%, at least 95%, at least 98%, at least 99%, or 100% aa sequence identity to the α1 and α2 domains (aas 1-181) of the DQA1*03:01 sequence depicted in FIG. 11, and ii) an MHC β chain polypeptide comprising an aa sequence having at least 90% or at least 95% aa sequence identity to the β1 and β2 domains (aas 1-188) of the DQB1*03:02 sequence depicted in FIG. 13.

A CIIC may comprise an MHC Class II α- and/or β-chain allele sequence that is associated with increased risk of developing T1D and/or celiac disease, such as where the patient or subject to be treated with the CIIC expresses the MHC Class II α- and/or β-chain allele.

### 2. Immunomodulatory Polypeptides

A CIIC may comprise one or more immunomodulatory polypeptides or “MODs.” MODs that are suitable for inclusion in a CIIC include, but are not limited to, IL-1, IL-2, IL-4, IL-6, IL-7, IL-10, IL-12, IL-15, IL-17, IL-21, IL-23, CD7, CD30L, CD40, CD70, CD80 (B7-1), CD83, CD86 (B7-2), HVEM (CD270), ILT3 (immunoglobulin-like transcript 3), ILT4 (immunoglobulin-like transcript 4), Fas ligand (FasL), ICAM (intercellular adhesion molecule), ICOS-L (inducible costimulatory ligand), JAG1 (CD339), lymphotoxin beta receptor, 3/TR6, OX40L (CD252), PD-L1, PD-L2, TGF-β1, TGF-β2, TGF-β3, 4-1BBL, and fragments of any thereof, such as ectodomain fragments, capable of engaging and signaling through their cognate receptor. In some cases, the MODs induce responses such as proliferation, activation and/or differentiation. In some cases, the MODs induce responses such as suppression/inhibition of proliferation, activation and/or differentiation. In some cases, the MODs can induce the formation, activation and/or proliferation of T regs. Some MODs suitable for inclusion in a CIIC, and their “co-MODS,” include polypeptide sequences with T cell modulatory activity from the protein pairs recited in Table 2:

TABLE 2
Exemplary Pairs of MODs and Co-MODs

| | |
|---|---|
| a) | 4-1BBL (MOD) and 4-1BB (Co-MOD), |
| b) | PD-L1 (MOD) and PD1 (Co-MOD), |
| c) | IL-2 (MOD) and IL-2 receptor (Co-MOD), |
| d) | CD80 (MOD) and CD28 (Co-MOD), |
| e) | CD86 (MOD) and CD28 (Co-MOD), |
| f) | OX40L (CD252) (MOD) and OX40 (CD134) |
| | (Co-MOD), |
| g) | Fas ligand (MOD) and Fas (Co-MOD), |
| h) | ICOS-L (MOD) and ICOS (Co-MOD), |
| i) | ICAM (MOD) and LFA-1 (Co-MOD), |
| j) | CD30L (MOD) and CD30 (Co-MOD), |
| k) | CD40 (MOD) and CD40L (Co-MOD), |
| l) | CD83 (MOD) and CD83L (Co-MOD), |
| m) | HVEM (CD270) (MOD) and CD160 (Co-MOD), |
| n) | JAG1 (CD339) (MOD) and Notch (Co-MOD), |
| o) | JAG1 (CD339) (MOD) and CD46 (Co-MOD), |
| p) | CD70 (MOD) and CD27 (Co-MOD), |
| q) | CD80 (MOD) and CTLA4 (Co-MOD), |
| r) | CD86 (MOD) and CTLA4 (Co-MOD), |
| s) | PD-L1(MOD) and CD-80 (Co-MOD), and |
| t) | TGF-β1, TGF-β2, and/or TGF-β3 |
| | (MODs), which may be masked, |
| | and TGF-β Receptor (e.g., TβRI |
| | and/or TβRII) (Co-MOD) |

In some cases, the MOD is selected from an IL-2 polypeptide, a 4-1BBL polypeptide, a B7-1 polypeptide, a B7-2 polypeptide, an ICOS-L polypeptide, an OX-40L polypeptide, a CD80 polypeptide, a CD86 polypeptide, a PD-L1 polypeptide, a FasL polypeptide, a TGFβ polypeptide, and a PD-L2 polypeptide. In some cases, the CIIC or duplex CIIC comprises two different MODs, such as an IL-2 MOD or IL-2 variant MOD and either a CD80 or CD86 MOD. In another instance, the CIIC or duplex CIIC comprises a wild-type or variant IL-2 MOD and a TGF-β MOD. In another instance, the CIIC or duplex CIIC comprises an IL-2 MOD or IL-2 variant MOD and a PD-L1 MOD. In some case MODs, which may be the same or different, are present in a CIIC or duplex CIIC in tandem. When MODs are presented in tandem, their sequences are immediately adjacent to each other on a single polypeptide, either without any intervening sequence or separated by only a linker polypeptide (e.g., no MHC sequences or epitope sequences intervene). The MOD may comprise all or part of the extracellular portion of a full-length MOD. Thus, for example, the MOD can in some cases exclude one or more of a signal peptide, a transmembrane domain, and an intracellular domain normally found in a naturally-occurring MOD. Unless stated otherwise, a MOD present in a CIIC or duplex CIIC does not comprise the signal peptide, intracellular domain, or a sufficient portion of the transmembrane domain to anchor a substantial amount (e.g., more than 5% or 10%) of a CIIC or duplex CIIC into a mammalian cell membrane.

In some cases, a MOD suitable for inclusion in a CIIC comprises all or a portion of (e.g., an extracellular portion of) the aa sequence of a naturally-occurring MOD. In other instances, a MOD suitable for inclusion in a CIIC is a variant MOD that comprises at least one aa substitution compared to the aa sequence of a naturally-occurring MOD. In some instances, a variant MOD exhibits a binding affinity for a co-MOD that is lower than the affinity of a corresponding naturally-occurring MOD (e.g., a MOD not comprising the aa substitution(s) present in the variant) for the co-MOD. Suitable variations in MOD sequences that alter affinity may be identified by scanning (making aa substitutions e.g., alanine substitutions or “alanine scanning,” or charged residue changes) along the length of a peptide, followed by testing the affinity of the resulting variants. Once key aa positions altering affinity are identified, those positions can be subject to a vertical scan in which the effect of one or more aa substitutions other than alanine are tested.

### a) MODs and Variant MODs

A MOD may comprise a wild-type aa sequence, or it may be a variant MOD that comprises, e.g., one or more (e.g., 1-20) aa substitutions, insertions, and/or deletions relative to a wild-type aa sequence. The MOD may comprise only the extracellular portion of a full-length immunomodulatory polypeptide. Alternatively, a MOD can comprise all or a portion of (e.g., an extracellular portion of) the aa sequence of a naturally-occurring MOD. A variant MOD may comprise 1-5 or 5-20 aa substitutions, insertions, and/or deletions relative to its wild-type MOD aa sequence (e.g., the sequence of the wild-type MOD's extracellular domain).

Variant MODs comprise at least one aa substitution, addition and/or deletion as compared to the aa sequence of a naturally-occurring immunomodulatory polypeptide. As noted above, in some instances a variant MOD exhibits a binding affinity for a co-MOD that is lower than the affinity of a corresponding naturally-occurring MOD (e.g., an immunomodulatory polypeptide not comprising the aa substitution(s) present in the variant) for the co-MOD.

MODs and variant MODs, including reduced affinity variants of proteins such as PD-L1, CD80, CD86, 4-1BBL and IL-2 are described in the published literature. For example, published PCT application WO2020132138Δ1 describes MODs and specific variants of MODs, including PD-L1, CD80, CD86, 4-1BBL, and IL-2 MODs described in paragraphs [00260]-[00455], which are hereby incorporated by reference.

Suitable immunomodulatory domains that exhibit reduced affinity for a co-immunomodulatory domain can have from 1 aa to 20 aa differences from a wild-type immunomodulatory domain. For example, in some cases, a variant MOD present in a CIIC may include a single aa substitution compared to a corresponding reference (e.g., wild-type) MOD. A variant MOD present in a CIIC may include 2 aa substitutions compared to a corresponding reference (e.g., wild-type) MOD. A variant MOD present in a CIIC may include 3 or 4 aa substitutions compared to a corresponding reference (e.g., wild-type) MOD. A variant MOD present in a CIIC may include 5 or 6 aa substitutions compared to a corresponding reference (e.g., wild-type) MOD. A variant MOD present in a CIIC may include 7, 8, 9 or 10 aa substitutions compared to a corresponding reference (e.g., wild-type) MOD. A variant MOD present in a CIIC may include 11-15 or 15-20 aa substitutions compared to a corresponding reference (e.g., wild-type) MOD.

As discussed above, a variant MOD suitable for inclusion in a CIIC may exhibit reduced affinity for a cognate co-MOD, compared to the affinity of a corresponding wild-type MOD for the cognate co-MOD.

Binding affinity between a MOD sequence and its cognate co-MOD can be determined by bio-layer interferometry (BLI) using the purified MOD sequence and purified cognate co-MOD, following the procedure set forth in published PCT Application WO 2020/132138 Δ1.

### (1) Masked TGF-β and its Variants

As discussed above, a CIIC may comprise at least one TGF-β polypeptide reversibly masked by a polypeptide (a “masking polypeptide”) that binds to the TGF-β polypeptide, which together form a masked TGF-β MOD. The masking polypeptide can be, for instance, a TGF-β receptor polypeptide or an antibody that functions to reversibly mask the TGF-β polypeptide present in the CIIC, where the TGF-β polypeptide is otherwise capable of acting as an agonist of a cellular TGF receptor. The masked TGF-β MODs provide active TGF-β polypeptides (e.g., TGF-β signaling pathway agonists). The TGF-β polypeptides and masking polypeptides (e.g., a TGF-β receptor fragment) interact with each other to reversibly mask the TGF-β polypeptide, thereby permitting the TGF-β polypeptide to interact with its cellular receptor. In addition, the masking sequence competes with cellular receptors that can scavenge TGF-β, such as the non-signaling TβRIII, thereby permitting the TGF-β MOD (and thus the CIIC) to effectively deliver active TGF-β agonist to target cells. While the CIIC constructs discussed herein permit epitope-specific presentation of a reversibly masked TGF-β to a target T cell, they also provide sites for the presentation of one or more additional MODs (e.g., IL-2). The ability of the CIIC construct to include one or more additional MODs thus permits the combined presentation of TGF-β and the additional MOD(s) to direct a target T cell's response in a substantially epitope-specific/selective manner in order to provide modulation of the target T cell. The CIIC thereby permits delivery of one or more masked TGF-β MODs in an epitope-selective (e.g., dependent/specific) manner that permits (i) formation of an active immune synapse with a target T cell, such as a CD4+ cell selective for the epitope, and (ii) modulation (e.g., control/regulation) of the target T cell's response to the epitope. Once engaged with the TCR of a T cell, the effect of a masked TGF-β MOD-containing CIIC on the T cell will depend on whether any additional MODs are present as part of the CIIC and, if so, which additional MOD(s) is/are present.

Further, although the CIICs of this disclosure may comprise both one or more masked TGF-β MODs and one or more additional MODs (e.g., wt. or variant IL-2, PD-L1, IL-10 and/or 4-1BB polypeptide aa sequences), if desired, the CIICs of this disclosure may comprise only one or more masked TGF-β MODs. That is, the one or more additional MODs such as wt. or variant IL-2, PD-L1 and/or IL-10 MODs need not be included in a CIIC of this disclosure along with a masked TGF-β MOD. The masked TGF-β MOD-containing CIICs can function as a means of producing TGF-β-driven T cell responses. For example, TGF-β by itself can inhibit the development of effector cell functions of T cells, activate macrophages, and/or promote tissue repair after local immune and inflammatory actions subside.

Although masked TGF-β MODs comprise a TGF-β polypeptide that is masked, the TGF-β polypeptide can still act as a TβR agonist because the TGF-β polypeptide-mask complex is reversible and “breathes” between an open state where the TGF-β polypeptide is available to cellular receptors, and a closed state where the mask engages the TGF-β polypeptide. The masking of the TGF-β polypeptide is reversible as a non-cleavable linker joins the mask to the TGF-β polypeptide or another peptide of the CIIC. That non-cleavable linker is not subject to site specific proteases (e.g., that give rise to a single cleavage in the linker) whose action on the linker would permit the mask to diffuse away from the TGF-β polypeptide. Accordingly, the masking polypeptide, which remains attached to the CIIC, functions to bind TGF-β polypeptide and prevent it from entering into tight complexes with, for example, ubiquitous non-signaling TβRIII molecules that can scavenge otherwise free TGF-β. Moreover, because the active forms of TGF-β are dimers that have higher affinity for TβRIII, substitutions that limit dimerization (e.g., a C77S substitution of the cysteine at position 77 with a serine) can be incorporated into TGF-β sequences in order to avoid scavenging by that receptor.

One effect of the masking sequence is to reduce the effective affinity of TGF-β1, TGF-β2, and TGF-β3 polypeptides for TβRs. At the same time, the affinity of the masking polypeptide for the TGF-β polypeptide can be altered so that it dissociates more readily from the TGF-β polypeptide, making the TGF-β polypeptide more available to cellular TβR proteins. That is, where the affinity of a masking polypeptide for a TGF-β polypeptide is reduced, the masked TGF-β MOD will spend more time in the open state. Although in the open state with the TGF-β polypeptide available for binding to cellular receptors, because the TβRII protein is generally the first peptide of the heteromeric TβRI/TβRII signaling complex to interact with TGF-β, control of the affinity of the TGF-β polypeptide for TβRII effectively controls entry of TGF-β into active signaling complexes. The incorporation of a substitution at, for example, one or more, two or more, or all three of Lys 25, Ile 92, and/or Lys 94 of TGF-β2 (or the corresponding positions of TGF-β1, TGF-β3) reduces affinity for TβRII polypeptides. The reduced affinity permits interactions between the target cell's TCR and the CIIC's MHC polypeptides and peptide epitope to effectively control binding and allows for target cell-specific interactions.

When a TβRII polypeptide is used as the masking polypeptide, the possibility of direct interactions with cellular TβRI receptors and off-target signaling can be addressed by appropriate modifications of the masking sequence. Where it is desirable to block/limit signaling by the masked TGF-β polypeptide through TβRI and/or modify (e.g., reduce) the affinity of a masking TβRII polypeptide for TGF-β, it is possible to incorporate N-terminal deletions and/or aa substitutions in the masking TβRII polypeptide. Modifications that can be made include deletions of N-terminal aas (e.g., N-terminal Δ14 or Δ25 deletions), and/or substitutions at one or more of L27, F30, D32, S49, I50, T51, S52, 153, E55, V77, D118, and/or E119. Some specific TβRII modifications resulting in a reduction in TβRI association with TβRII and reduced affinity for TGF-β include any one or more of L27A, F30A, D32A, D32N, S49A, 150A, T51A, S52A, S52L, 153A, E55A, V77A, D118A, D118R, E119A, and/or E119Q.

The TGF-β polypeptide present in a CIIC is in some cases a variant TGF-β polypeptide, including a variant TGF-β polypeptide that has a lower affinity for at least one class of TGF-β receptors, or is selective for at least one class of TGF-β receptors, compared to a wild-type TGF-β polypeptide.

While a TGF-β1 polypeptide, a TGF-β2 polypeptide, or a TGF-β3 polypeptide can be incorporated into a CIIC as part of a masked TGF-β polypeptide, a variety of factors may influence the choice of the specific TGF-β polypeptide, and the specific sequence and aa substitutions that will be employed. For example, TGF-β1 and TGF-13 polypeptides are subject to “clipping” of their aa sequences when expressed in certain mammalian cell lines (e.g., CHO cells). In addition, dimerized TGF-β (e.g., TGF-β2) has a higher affinity for the TβRIII (beta glycan receptor) than for the TβRII I receptor, which could lead to off target binding and loss of biologically active masked protein to the large in vivo pool of non-signaling TβRIII molecules. To minimize high-affinity off target binding to TβRIII, it may be desirable to substitute the residues leading to dimeric TGF-β molecules, which are joined by a disulfide bond. Accordingly, cysteine 77 (C77) may be substituted by an aa other than cysteine (e.g., a serine forming a C77S substitution).

Amino acid sequences of TGF-β polypeptides are known in the art. In some cases, the TGF-β polypeptide present in a masked TGF-β polypeptide is a TGF-β1 polypeptide. In some cases, the TGF-β polypeptide present in a masked TGF-β polypeptide is a TGF-β2 polypeptide. In some cases, the TGF-β polypeptide present in a masked TGF-β polypeptide is a TGF-β3 polypeptide.

A suitable TGF-β polypeptide can have a length from about 70 aas to about 125 aas, for example, a suitable TGF-β polypeptide can have a length from about 70 aas to about 80 aas, from about 80 aas to about 90 aas, from about 90 aas to about 100 aas, from about 100 aas to about 105 aas, from about 105 aas to about 110 aas, from about 110 aas to about 112 aas, from about 113 aas to about 120 aas, or from about 120 aas to about 125 aas. A suitable TGF-β polypeptide can comprise an aa sequence having at least 85%, at least 90%, at least 95%, at least 98%, at least 99%, or 100% aa sequence identity to at least 80, at least 90, at least 100, or at least 110 contiguous aas of the mature form of a human TGF-β1 polypeptide, a human TGF-β2 polypeptide, or a human TGF-13 polypeptide.

### (a) TGF-β1 Polypeptides

A suitable TGF-β1 polypeptide may comprise an aa sequence having at least 85%, at least 90%, at least 95%, at least 98%, at least 99%, or 100% aa sequence identity to at least 70, at least 80, at least 90, at least 100, at least 110, or 112 aas of the following TGF-β1 aa sequence: AL DTNYCFSSTE KNCCVRQLYI DFRKDLGWKW IHEPKGYHAN FCLGPCPYIW SLDTQYSKVL ALYNQHNPGA SAAPCCVPQA LEPLPIVYYV GRKPKVEQLS NMIVRSCKCS (SEQ ID NO:202), where the TGF-β1 polypeptide has a length of about 112 aas. A TGF-β1 preproprotein is provided in FIG. 2I as SEQ ID NO:157. Amino acids R25, C77, V92 and R94 are bolded and italicized. See FIG. 21.

In some cases, a suitable TGF-β1 polypeptide comprises a C77S substitution. Thus, in some cases, a suitable TGF-β1 polypeptide comprises an aa sequence having at least 85%, at least 90%, at least 95%, at least 98%, at least 99%, or 100% aa sequence identity to at least 70, at least 80, at least 90, at least 100, at least 110, or 112 aas of the following TGF-β1 aa sequence: AL DTNYCFSSTE KNCCVRQLYI DFRKDLGWKW IHEPKGYHAN FCLGPCPYIW SLDTQYSKVL ALYNQHNPGA SAAPSCVPQA LEPLPIVYYVGRKPKVEQLS NMIVRSCKCS (SEQ ID NO:203), where aa 77 is Ser. Positions 25, 77, 92 and 94 are bolded and italicized.

### (b) TGF-β2 Polypeptides

A suitable TGF-β2 polypeptide can comprise an aa sequence having at least 85%, at least 90%, at least 95%, at least 98%, at least 99%, or 100% aa sequence identity to at least 70, at least 80, at least 90, at least 100, at least 110, or 112 aas of the following TGF-β2 aa sequence: ALDAAYCFR NVQDNCCLRP LYIDFKRDLG WKWIHEPKGY NANFCAGACP YLWSSDTQHS RVLSLYNTIN PEASASPCCV SQDLEPLTIL YYIGKTPKIE QLSNMIVKSC KCS (SEQ ID NO:204), where the TGF-β2 polypeptide has a length of about 112 aas. A TGF-β2 preproprotein is provided in FIG. 21 as SEQ ID NO:159. Residues Lys 25, Cys 77, Ile 92, and Lys 94 are bolded and italicized.

In some cases, a suitable TGF-β2 polypeptide comprises a C77S substitution. Thus, in some cases, a suitable TGF-β2 polypeptide comprises an aa sequence having at least 85%, at least 90%, at least 95%, at least 98%, at least 99%, or 100% aa sequence identity to at least 70, at least 80, at least 90, at least 100, at least 110, or 112 aas of the following TGF-β2 aa sequence: ALDAAYCFR NVQDNCCLRP LYIDFKRDLG WKWIHEPKGY NANFCAGACP YLWSSDTQHS RVLSLYNTIN PEASASPSCV SQDLEPLTIL YYIGKTPKIE QLSNMIVKSC KCS (SEQ ID NO:118), which is SEQ ID NO:204 in wherein Cys 77 is substituted by a Ser (C77S) that is bolded and italicized.

### (c) TGF-β3 Polypeptides

A suitable TGF-β3 polypeptide can comprise an aa sequence having at least 85%, at least 90%, at least 95%, at least 98%, at least 99%, or 100% aa sequence identity to at least 70, at least 80, at least 90, at least 100, at least 110, or 112 aas of the following TGF-β3 aa sequence: ALDTNYCFRN LEENCCVRPL YIDFRQDLGW KWVHEPKGYY ANFCSGPCPY LRSADTTHST VLGLYNTLNP EASASPCCVP QDLEPLTILY YVGRTPKVEQ LSNMVVKSCK CS (SEQ ID NO:161), where the TGF-β3 polypeptide has a length of about 112 aas. A TGF-β3 isoform 1 preproprotein is provided in FIG. 21 as SEQ ID NO:160. Positions 25, 77, 92 and 94 are bolded and italicized.

In some cases, a suitable TGF-β3 polypeptide comprises a C77S substitution. In some cases, a suitable TGF-β3 polypeptide comprises an aa sequence having at least 85%, at least 90%, at least 95%, at least 98%, at least 99%, or 100% aa sequence identity to at least 70, at least 80, at least 90, at least 100, at least 110, or 112 aas of the following TGF-β3 aa sequence: ALDTNYCFRN LEENCCVRPL YIDFRQDLGW KWVHEPKGYY ANFCSGPCPY LRSADTTHST VLGLYNTLNP EASASPSCVP QDLEPLTILY YVGRTPKVEQ LSNMVVKSCK CS (SEQ ID NO:162), where aa 77 is Ser. Positions 25, 77, 92 and 94 are bolded and italicized.

### (d) Additional TGF-β Polypeptide Sequence Variations

In addition to sequence variations that alter TGF-β molecule dimerization (e.g., cysteine 77 substitutions such as C77S), TGF-β1, TGF-β2, and TGF-β3 polypeptides having sequence variations that affect affinity and other properties may be incorporated into a masked TGF-β MOD. When a variant TGF-β with reduced affinity for the masking polypeptide (e.g., a TβR polypeptide such as a TβRII polypeptide) is present in the masked TGF-β MOD those components dissociate more readily, making the TGF-β polypeptide more available to cellular TβR proteins. Because the TβRII protein is generally the first peptide of the heteromeric TβR signaling complex to interact with TGF-β, interactions with TβRII effectively controls entry of TGF-β into active signaling complexes. Accordingly, variants controlling the affinity of TGF-β for TβRII may effectively control entry of masked TGF-β MODs into active signaling complexes.

The present disclosure includes and provides for masked TGF-β MODs comprising a variant masking TβR (e.g., TβRII) polypeptide sequence and/or a variant TGF-β polypeptide having altered (e.g., reduced) affinity for each other (relative to an otherwise identical masked TGF-β MOD without the sequence variation(s)). Affinity between a TGF-β polypeptide and a TβR (e.g., TβRII) polypeptide may be determined using BLI as described above for MODs and their co-MODs.

### (i) Additional TGF-β2 Sequence Variants

The present disclosure includes and provides for masked TGF-β2 MODs comprising a masking TβR (e.g., TβRII) polypeptide sequence and either a wt. or a variant TGF-β2 polypeptide, where the variant polypeptide has a reduced affinity for the masking TβR (relative to an otherwise identical wt. TGF-β polypeptide sequence without the sequence variations).

The disclosure provides for masked TGF-β MODs that comprise a masking TβRII receptor sequence and a variant TGF-β2 polypeptide having greater than 85% (e.g., greater than 90%, 95%, 98% or 99%) sequence identity to at least 100 contiguous aas of SEQ ID NO:159, and comprising a substitution reducing the affinity of the variant TGF-β2 polypeptide for the TβRII I receptor sequence.

In some cases, a masked TGF-β MOD comprises a masking TβRII polypeptide and a variant TGF-β (e.g., TGF-β2) polypeptide comprising a substitution at one or more, two or more, or all three of Lys 25, Ile 92, and/or Lys 94 (see the mature form of TGF-β2 in SEQ ID NO:159 for the location of the residues, and FIG. 2I for the corresponding residues in the mature forms of TGF-β1 and TGF-β3). Those aa residues have been shown to affect the affinity of TGF-β2 for TβRII polypeptides (see De Crescenzo et al., J. Mol. Biol. 355: 47-62 (2006)). The CIIC optionally comprises one or more independently selected MODs such as IL-2 or a variant thereof. In one instance, the masked TGF-β MOD comprises a masking TβRII polypeptide and a TGF-β2 polypeptide having an aa other than Lys or Arg at position 25 of SEQ ID NO:159, with the CIIC optionally comprising one or more additional independently selected MODs (e.g., one or more IL-2 MODs or reduced affinity variants thereof). A masked TGF-β MOD with a masking TβRII polypeptide may comprise a TGF-β2 polypeptide having an aa other than Ile or Val at position 92 of SEQ ID NO:159 (or an aa other than Ile, Val, or Leu at position 92), with the CIIC optionally comprising one or more additional independently selected MODs (e.g., one or more IL-2 MODs or reduced affinity variants thereof). A masked TGF-β MOD with a masking TβRII polypeptide may comprise a TGF-β2 polypeptide having an aa other than Lys or Arg at position 94 of SEQ ID NO:159), with the CIIC optionally comprising one or more additional independently selected MODs (e.g., one or more IL-2 MODs or reduced affinity variants thereof). A masked TGF-β MOD with a masking TβRII polypeptide may comprise a TGF-β2 polypeptide comprising a substitution at one or more, two or more or all three of Lys 25, Ile 92, and/or Lys 94), with the CIIC optionally comprising one or more additional independently selected MODs. A masked TGF-β MOD with a masking TβRII polypeptide may comprise a TGF-β2 polypeptide comprising a substitution at one or more, two or more or all three of Lys 25, Ile 92, and/or Lys 94), with the CIIC optionally comprising one or more independently selected IL-2 MODs or reduced affinity variants thereof.

### (ii) Additional TGF-β1 and TGF-β3 Sequence Variants and Placement in Tandem

In some cases, a masked TGF-β MOD comprises a masking TβRII polypeptide and a variant TGF-β1 or TGF-β3 polypeptide comprising a substitution at one or more, two or more or all three aa positions corresponding to Lys 25, Ile 92, and/or Lys 94 in the mature TGF-β2 polypeptide of SEQ ID NO:159. In the mature TGF-β1 or TGF-β3 polypeptides, the aa that corresponds to: Lys 25 is Arg 25, Ile 92 is Val 92, and Lys 94 is Arg 94, each of which is a conservative substitution. See, e.g., SEQ ID NOs:157, 158, 202, and 203 for TGF-β1, and SEQ ID NOs:160, 161, and 162 for TGF-β3.

As noted above, the masked TGF-β MOD optionally comprises one or more independently selected MODs such as IL-2 or a variant thereof. In one instance, the masked TGF-β MOD with a masking TβRII polypeptide comprises a TGF-β1 or β3 polypeptide having an aa other than Arg or Lys at position 25, and optionally comprises one or more independently selected MODs (e.g., one or more IL-2 MODs or reduced affinity variants thereof). In one instance, the masked TGF-β MOD with a masking TβRII I polypeptide comprises a TGF-β1 or 13 polypeptide having an aa other than Val or lie at position 92 (or an aa other than Ile, Val, or Leu at position 92), and optionally comprises one or more independently selected MODs (e.g., one or more IL-2 MODs or reduced affinity variants thereof). In another instance, the masked TGF-β MOD with a masking TβRII polypeptide comprises a TGF-β2 polypeptide having an aa other than Arg or Lys, and optionally comprises one or more independently selected MODs (e.g., one or more IL-2 MODs or reduced affinity variants thereof). In one specific instance, a masked TGF-β MOD with a masking TβRII polypeptide comprises a TGF-β1 or β3 polypeptide comprising a substitution at one or more, two or more or all three of Arg 25, Val 92, and/or Arg 94, and further comprises one or more independently selected MODs (e.g., IL-2 or variant IL-2 MODs). In another specific instance, a masked TGF-β MOD with a masking TβRII polypeptide comprises a TGF-β1 or β3 polypeptide comprising a substitution at one or more, two or more or all three of Arg 25, Val 92, and/or Arg 94, and further comprises one or more independently selected IL-2 MODs, or reduced affinity variants thereof.

(e) TGF-β Receptor Polypeptides and Other Polypeptides that Bind and Mask TGF-β

In any of the above-mentioned TGF-β polypeptides or polypeptide complexes the polypeptide that binds to and masks the TGF-β polypeptide (the “masking polypeptide”) can take a variety of forms, including fragments of TβRI, TβRII, TβRIII and anti-TGF-β antibodies or antibody-related molecules (e.g., antigen binding fragment of an antibody, Fab, Fab′, single chain antibody, scFv, peptide aptamer, or nanobody).

### (f) TGF-β Receptor Polypeptides

The masking of TGF-β in masked TGF-β MODs may be accomplished by utilizing a TGF-β receptor fragment (e.g., the ectodomain sequences of TβRI, TβRII or TβRIII) that comprises polypeptide sequences sufficient to bind a TGF-β polypeptide (e.g., TGF-β1, TGF-β2 or TGF-β3). In an embodiment, the masking sequence comprises all or part of the TβRI, TβRII, or TβRIII ectodomain.

### (i) TGF-β Receptor I (TβRI)

The polypeptide sequence masking TGF-β in a masked TGF-β MOD may be derived from a TβRI (e.g., isoform 1, SEQ ID NO:163, see FIG. 23A) and may comprise all or part of the TβRI ectodomain (aas 34-126). A suitable TβRI polypeptide for masking TGF-β may comprise an aa sequence having at least 85%, at least 90%, at least 95%, at least 98%, at least 99%, or 100% aa sequence identity to at least 70, at least 80, at least 90, at least 100, or 103 aas of the following TβRI ectodomain aa sequence: LQCFCHL CTKDNFTCVT DGLCFVSVTE TTDKVIHNSM CIAEIDLIPR DRPFVCAPSS KTGSVTTTYC CNQDHCNKIE LPTTVKSSPG LGPVEL (SEQ ID NO:164).

### (ii) TGF-β Receptor II (TβRII)

A polypeptide sequence masking TGF-β in a masked TGF-β MOD may be derived from a TβRII (e.g., isoform A, SEQ ID NO:165), and may comprise all or part of the TβRII ectodomain sequence (aas 24 to 177). A suitable TβRII isoform A polypeptide for masking TGF-β may comprise an aa sequence having at least 85%, at least 90%, at least 95%, at least 98%, at least 99%, or 100% aa sequence identity to at least 70, at least 80, at least 90, at least 100, at least 110, at least 120, at least 130, at least 140, at least 150 or at least 154 aas of the following TβRII isoform A ectodomain aa sequence: IPPHVQK SDVEMEAQKD EIICPSCNRT AHPLRHINND MIVTDNNGAV KFPQLCKFCD VRFSTCDNQK SCMSNCSITS ICEKPQEVCV AVWRKNDENI TLETVCHDPK LPYHDFILED AASPKCIMKE KKKPGETFFM CSCSSDECND NIIFSEE (SEQ ID NO:166). The location of the aspartic acid residue corresponding to D118 in the B isoform is bolded and italicized.

A polypeptide sequence masking TGF-β in a masked TGF-β MOD may be derived from TβRII isoform B (SEQ ID NO:167) and may comprise all or part of the TβRII ectodomain sequence (aas 24 to 166). A suitable TβRII isoform B polypeptide for masking TGF-β may comprise an aa sequence having at least 85%, at least 90%, at least 95%, at least 98%, at least 99%, or 100% aa sequence identity to at least 70, at least 80, at least 90, at least 100, at least 110, at least 120, at least 130, at least 140, or 143 aas of the TβRII isoform B ectodomain aa sequence: IPPHVQKSVN NDMIVTDNNG AVKFPQLCKFCDVRFSTCDN QKSCMSNCSI TSICEKPQEV CVAVWRKNDE NITLETVCHD PKLPYHDFIL EDAASPKCIM KEKKKPGETF FMCSCSSDEC NDNIIFSEEY NTSNPDLLLV IFQ (SEQ ID NO:168). As discussed below, any one or more of F30, D32, S52, E55, or D118 (italicized and bolded) may be substituted by an aa other than the aa occurring at those positions in the sequence provided (e.g., alanine). A polypeptide sequence masking TGF-β may comprise the polypeptide of SEQ ID NO:168 bearing a D118A or D118R substitution. A sequence masking TGF-β may comprise the peptide of SEQ ID NO:168 bearing a D118A or D118R substitution and one or more of a F30A, D32N, S52L and/or E55A substitution.

Although TβRII's ectodomain may be utilized as a masking polypeptide, that region of the protein has charged and hydrophobic patches that can lead to unfavorable isoelectric points (pI values) and can be toxic to cells expressing the polypeptide. In addition, combining a TβRII ectodomain with an active TGF-β polypeptide can result in a complex that could combine with cell surface TβRI and cause activation of that signaling receptor (e.g., signaling through the Smad pathway). Modifying TβRII ectodomain sequences used to mask TGF-β by removing or altering sequences involved in TβRI association can avoid the unintentional stimulation of cells by the masked TGF-β except through their own cell surface heterodimeric TβRI/TβRII complex. Modifications of TβRII may also alter (e.g., reduce) the affinity of the TβRII for TGF-β (e.g., TGF-β3), thereby permitting control of TGF-β unmasking and its availability as a signaling molecule. Masked TGF-β MODs comprising TβR (e.g., TβRII) peptides with the highest affinity for TGF-β (e.g., TGF-β3) most tightly mask the TGF-β sequence and require higher doses to achieve the same effect. In contrast, aa substitutions in TβRII that lower the affinity unmask the TGF-β polypeptide and are biologically effective at lower doses.

Accordingly, where it is desirable to block/limit signaling by the masked TGF-β polypeptide through TβRI and/or modify (e.g., reduce) the affinity of a masking TβRII I polypeptide for TGF-β, a number of alterations to TβRII I may be incorporated into the TβRII polypeptide sequence. Modifications that can be made include the above-mentioned deletions of N-terminal aas, such as 14 or 25 N-terminal aas (from 1 to 14 aas or from 1 to 25 aas, Δ14, A25 modifications), and/or substitutions at one or more of L27, F30, D32, S49, 150, T51, S52, 153, E55, V77, D118, and/or E119. Some specific TβRII modifications resulting in a reduction in TβRI association with TβRII and reduced affinity for TGF-β include any one or more of L27A, F30A, D32A, D32N, S49A, 150A, T51A, S52A, S52L, 153A, E55A, V77A, D118A, D118R, E119A, and/or E119Q based on SEQ ID NO:168. See, e.g., J. Groppe et al. Mol Cell 29, 157-168, (2008) and De Crescenzo et al. J Mol Biol 355, 47-62 (2006) for the effects of those substitutions on TGF-13-TβRII and TIRI-TβRII complexes. Modifications of TβRII including an N-terminal 625 deletion and/or substitution at F24 (e.g., an F24A substitution) substantially or completely block signal through the canonical SMAD signaling pathway. In one aspect, the aspartic acid at position 118 (D118) of the mature TβRII B isoform (SEQ ID NO:168) is replaced by an aa other than Asp or Glu, such as Ala, giving rise to a “D118A” substitution or by an Arg giving rise to a D118R substitution. The Asp residues corresponding to D118 are indicated in SEQ ID NOs:168-172 (with bold and underlining in FIG. 23B). N-terminal deletions of from 1 to 25 aas in length (e.g., a Δ25 deletion) and/or substitution at F24 (e.g., an F24A substitution) may be combined with D118 substitutions (e.g., D118A or D118R). N-terminal deletions of from 1 to 25 aas in length (e.g., a Δ25 deletion) and/or substitution at F24 (e.g., an F24A substitution) may also be combined with substitutions at any of L27, F30, D32, S49, 150, T51, S52, 153, E55, V77, D118, and/or E119 (e.g., D118A) substitutions, and particularly any of the specific substitutions recited for those locations in SEQ ID NO:168 described above to alter the affinity.

Deletions of the N-terminus of the TβRII polypeptides may also result in loss of TβRI interactions and prevent masked TGF-β MODs comprising a TβRII polypeptide from acting as a constitutively active complex that engages and activates TβRI signaling. A 14 aa deletion (A14) of the TβRII polypeptide substantively reduces the interaction of the protein with TβRI, and a Δ25 aa deletion of TβRII appears to completely abrogate the interaction with TβRI. N-terminal deletions also substantially alter the β1 of the protein, with the Δ14 TβRII ectodomain mutant displaying a β1 of about 4.5-5.0 (e.g., about 4.74). Accordingly, TGF-β MODs may comprise TβRII ectodomain polypeptides (e.g., polypeptides of SEQ ID NOs:168 or 169) with N-terminal deletions, such as from 14 to 25 aas, including, e.g., 14, 15, 16, 17, 18, 19, 20, 21, 22, 23, 24, or 25 aas. Modified ectodomain sequences, including those that limit interactions with TβRI, that may be utilized to mask TGF-β polypeptides in a masked TGF-β MOD are described in the paragraphs that follow.

In an embodiment, the sequence masking TGF-β in a masked TGF-β MOD comprises an aa sequence having at least 85%, at least 90%, at least 95%, at least 98%, at least 99%, or 100% aa sequence identity to at least 70, at least 80, at least 90, at least 100, at least 110, at least 120, at least 130, at least 140, or 142 aas of the TβRII isoform B ectodomain sequence: IPPHVQKSVN NDMIVTDNNG AVKFPQLCKFCDVRFSTCDN QKSCMSNCSI TSICEKPQEV CVAVWRKNDE NITLETVCHD PKLPYHDFIL EDAASPKCIM KEKKKPGETF FMCSCSSDEC NDNIIFSEE (SEQ ID NO:169). Any one or more of F30, D32, S52, E55, or D118 (italicized and bolded) may be substituted by an aa other than the aa occurring at those positions in the sequence provided (e.g., alanine). In an embodiment, the sequence masking TGF-β comprises the polypeptide of SEQ ID NO:169 bearing a D118A substitution. In an embodiment, the sequence masking TGF-β comprises the polypeptide of SEQ ID NO:169 bearing a D118A substitution and one or more of a F30A, D32N, S52L and/or E55A substitution.

Combinations of N-terminal deletions of TβRII, such as from 14 to 25 aas, including, e.g., 14, 15, 16, 17, 18, 19, 20, 21, 22, 23, 24, or 25 aas, that block inadvertent cell signaling due to the masked TGF-β/TβRII complex interacting with TβRI may be combined with other TβRII ectodomain substitutions, including those at any one or more of F30, D32, S52, E55, and/or D118. The combination of deletions and substitutions ensures the masked TGF-1 MOD does not cause cell signaling except through the cell's membrane bound TβRI & TβRII receptors.

In an embodiment, the sequence masking TGF-β in a masked TGF-β MOD comprises an aa sequence having at least 85%, at least 90%, at least 95%, at least 98%, at least 99%, or 100% aa sequence identity to at least 70, at least 80, at least 90, at least 100, at least 110, or 114 aas of the TβRII isoform B ectodomain sequence: VTDNNG AVKFPQLCKFCDVRFSTCDN QKSCMSNCSI TSICEKPQEV CVAVWRKNDE NITLETVCHD PKLPYHDFIL EDAASPKCIM KEKKKPGETF FMCSCSSDEC NDNIIFSEE (SEQ ID NO:205), which has aas 1-14 (A14) deleted. Any one or more of F30, D32, S52, E55, or D118 (italicized and bolded) may be substituted by an aa other than the aa occurring at those positions in the sequence provided (e.g., alanine). In an embodiment, the sequence masking TGF-β comprises the peptide of SEQ ID NO:205 bearing a D118A substitution (see SEQ ID NO:170 in FIG. 23B). In an embodiment, the sequence masking TGF-β comprises the polypeptide of SEQ ID NO:205 bearing a D118A substitution and one or more of a F30A, D32N, S52L and/or E55A substitution.

In an embodiment, the sequence masking TGF-β in a masked TGF-β MOD comprises an aa sequence having at least 85%, at least 90%, at least 95%, at least 98%, at least 99%, or 100% aa sequence identity to at least 70, at least 80, at least 90, at least 100, or 104 aas of the TβRII isoform B ectodomain sequence: QLCKF CDVRFSTCDN QKSCMSNCSI TSICEKPQEV CVAVWRKNDE NITLETVCHD PKLPYHDFIL EDAASPKCIM KEKKKPGETF FMCSCSSDEC NDNIIFSEE (SEQ ID NO:206), which has aas 1-25 (A25) deleted. Any one or more of F30, D32, S52, E55, or D118 (italicized and bolded) may be substituted by an aa other than the aa occurring at those positions in the sequence provided (e.g., alanine). In an embodiment, the sequence masking TGF-β comprises the polypeptide of SEQ ID NO:206 bearing a D118A substitution (shown as SEQ ID NO:172 in FIG. 23B). In an embodiment, the sequence masking TGF-β in a masked TGF-β MOD comprises the polypeptide of SEQ ID NO:206 bearing a D118A substitution and one or more of F30A, D32N, S52L and/or E55A substitutions. In an embodiment, the sequence masking TGF-β in a masked TGF-β MOD comprises the polypeptide of SEQ ID NO:206 (see FIG. 23B) bearing D118A and F30A substitutions. In an embodiment, the sequence masking TGF-β in a masked TGF-β MOD comprises the polypeptide of SEQ ID NO:206 (see FIG. 23B) bearing D118A and D32N substitutions. In an embodiment, the sequence masking TGF-β in a masked TGF-β MOD comprises the polypeptide of SEQ ID NO:206 (see FIG. 23B) bearing D118A and S52L substitutions. In an embodiment, the sequence masking TGF-β in a masked TGF-β MOD comprises the peptide of SEQ ID NO:206 (see FIG. 23B) bearing D118A and E55A.

(iii) TGF-β Receptor III (TβRIII)

In an embodiment, the polypeptide sequence masking TGF-β in a masked TGF-β MOD may be derived from a TβRIII (e.g., isoform A, SEQ ID NO:173 and isoform B), and may comprise all or part of a TβRIII ectodomain (aas 27-787 of the A isoform or 27-786 of the B isoform). In some cases, a suitable TβRIII polypeptide for masking TGF-β comprises an aa sequence having at least 85%, at least 90%, at least 95%, at least 98%, at least 99%, or 100% aa sequence identity to at least 70, at least 80, at least 90, at least 100, or 120 aas of a TβRIII A isoform or B isoform ectodomain sequence (e.g., provided in FIG. 23C as SEQ ID NO:173 or SEQ ID NO:174).

### (g) Antibodies

Although TGF-β receptor polypeptides (e.g., ectodomain sequences) can function to bind and mask TGF-β polypeptides in masked TGF-β MODs, other polypeptide sequences (protein sequences) that bind to TGF-β sequences can also be employed as masking polypeptides. Among the suitable polypeptide or protein sequences that can be used to mask TGF-β are antibodies with affinity for TGF-β (e.g., antibodies specific for one or more of TGF-β1, TGF-β2, or TGF-β3) or antibody-related molecules such as anti-TGF-β antibody fragments, nanobodies with affinity for TGF-β polypeptides, and particularly single chain anti-TGF-β antibodies (e.g., any of which may be humanized). Some antibodies, including scFV antibodies that bind and neutralize TGF-β, have been described. See e.g., U.S. Pat. No. 9,090,685. Throughout the embodiments and/or aspects of the invention described in this disclosure, TβR (e.g., TβRII) sequences used to mask TGF-β polypeptides may be replaced with masking antibody sequences (e.g., scFV or a nanobody) with affinity for the TGF-β polypeptide. For instance, in each of the masked TGF-β MODs in FIG. 1 (see structures R to U) where a TGF-β receptor sequence is used to mask a TGF-β polypeptide, the receptor polypeptide may be replaced with a masking antibody polypeptide (e.g., scFV or a nanobody) with affinity for the TGF-β polypeptide.

One potential advantage of using an antibody (e.g., a single chain antibody) as a masking polypeptide is the ability to limit it to the isoform of the TGF-β polypeptide(s) to be masked. By way of example, single chain antibody sequences based on Metelimumab (CAT192) directed against TGF-β1 (e.g., Lord et al., mAbs 10(3): 444-452 (2018)) can be used to mask that TGF-β isoform when present in TGF-β MODs. In another embodiment, a single chain antibody sequence specific for TGF-β2 is used to mask that TGF-β isoform when present in TGF-β MODs. In another embodiment, a single chain antibody sequence specific for TGF-β3 is used to mask that TGF-β isoform when present in TGF-β MODs. Single chain antibodies can also be specific for a combination of TGF-β isoforms (e.g., ectodomain sequences appearing in masked TGF-β MODs selected from the group consisting of: TGF-β1 and TGF-β2; TGF-β1 and TGF-β3; and TGF-β2 and TGF-β3). The single chain antibodies may also be pan-specific for TGF-β1, TGF-β2, and TGF-β3 ectodomain sequences appearing in masked TGF-β MODs See e.g., WO 2014/164709. Antibodies and single chain antibodies that have the desired specificity and affinity for TGF-β isoforms can be prepared by a variety of methods, including screening hybridomas and/or modification (e.g., combinatorial modification) to the variable region sequence of antibodies that have affinity for a target TGF-β polypeptide sequence.

In an embodiment, a masked TGF-β MOD comprises a single chain antibody to mask a TGF-β sequence (e.g., a TGF-β3 sequence). In one such embodiment the single chain aa sequence is specific for the TGF-β3 set forth in SEQ ID NO:161 comprising a C77S substitution (see SEQ ID NO:162).

### (h) Placement of TGF-β and TGF-β Masking Sequence in CIICs

The masking sequence (e.g., a TGF-β receptor sequence) of a masked TGF-β MOD may be part of the same polypeptide as the TGF-β sequence; that is, both the masking and TGF-β sequences are present in “cis.” Alternatively, the masking sequence (e.g., a TGF-β receptor sequence) and the TGF-β sequence may be part of different polypeptides, which is to say they are present in “trans.”

When the masking sequence and the TGF-β sequence of a masked TGF-β MOD are present in a single aa sequence (single polypeptide) of a CIIC (placed in cis, e.g., as in FIG. 1, structures R and S), the aa sequence may be arranged in the N-terminal to C-terminal direction as either: a) TGF-β receptor sequence(s) followed by TGF-β sequence(s), or b) TGF-β sequence(s) followed by TGF-β receptor sequence(s). Regardless of the order from N-terminus to C-terminus, the polypeptide sequence of a masked TGF-β MOD may be linked to any other CIIC polypeptide at its N-terminus or C-terminus. Independently selected linker polypeptide(s) (e.g., Gly4Ser repeats) may be used to join the masking sequence (e.g., a TGF-β receptor sequence) and the TGF-β sequence, and also to join the TGF-β MOD to a polypeptide of the CIIC (e.g., a scaffold polypeptide sequence). As an example, a cis-masked TGF-β MOD may be linked to the C terminus of a CIIC as a single aa sequence (polypeptide) and have the order from N-terminus to C-terminus of a) TGF-β receptor sequence (e.g., a TβRII sequence) followed by TGF-β sequence (e.g., TGF-β3). To further that example, the cis-masked TGF-β MOD may be linked to a scaffold polypeptide (e.g., C-terminal to the CIIC α2 domain) and the cis-masked TGF-β MOD may optionally be followed by another MOD such as IL-2.

One example of a masked TGF-β MOD with the TβR and TGF-β in cis (a cis-masked TGF-β MOD) is the sequence: QLCKFCDVRFSTCDNQKSCMSNCSITSICEKPQEVCVAVWRKNDENITLETVCHDPKLPYHDFILEDAAS PKCIMKEKKKPGETFFMCSCSSAECNDNIIFSEEYNTSNPDGGGGSGGGGSGGGGSGGGGSGGGGSALDTNYCF RNLEENCCVRPLYIDFRQDLGWKWVHEPKGYYANFCSGPCPYLRSADTTHSTVLGLYNTLNPEASASPSCVPQDLE PLTILYYVGRTPKVEQLSNMVVKSCKCS (SEQ ID NO:207), where: aas 1-111 are a human TβRII masking sequence with the N-terminal 25 aas removed (625) and a D118A substitution, aas 112-136 are a linker (five Gly4Ser repeats), and 137-248 is a human TGF-β3 sequence with a C77S substitution. Such a sequence may be attached, for example, by its N-terminus, directly or indirectly via an independently selected linker, to the C-terminus of a CIIC as a single aa sequence (polypeptide) (e.g., a scaffold polypeptide C-terminal to the α2 domain sequence of a CIIC). In addition, the cis masked TGF-β MOD sequence may have appended to it another MOD sequence (e.g., a human IL-2 or variant IL-2 MOD sequence).

When the masking sequence (e.g., TGF-β receptor sequence) and the TGF-β sequence of a masked TGF-1 MOD are present as part of different CIIC polypeptides (placed in trans), those polypeptide sequences are attached to different (separate) CIIC polypeptides that interact, thereby pairing the TGF-β sequence with the masking polypeptide (e.g., a TGF-β receptor sequence). The TGF-β sequence and masking sequence may be located at the C-terminus of CIIC polypeptides (e.g., at the C-terminus of a scaffold sequence such as an Ig Fc scaffold; see FIG. 1, structures R-U and W). Independently selected linker polypeptide(s) (e.g., Gly₄Ser (SEQ ID NO:237) repeats) may be used to join the masking sequence (e.g., TGF-β receptor sequence) or the TGF-β sequence to other CIIC polypeptides. As an example, in a trans-masked TGF-β MOD a TGF-β receptor sequence (e.g., TβRII) may be located on one scaffold of a duplex CIIC and the TGF-β sequence (e.g., TGF-β3) may be part of a second scaffold polypeptide, where the first and second scaffold polypeptides associate through interspecific multimerization sequences (see, e.g., FIG. 1, structures T and U). To further that example, in a CIIC heteroduplex the TGF-β sequence and TGF-β receptor sequence may be located at the C-terminus of different scaffold polypeptide sequences (e.g., an Ig Fc sequence) and may optionally be followed by another MOD such as IL-2. By way of example, a duplex CIIC having first and second scaffold polypeptides with interspecific multimerization sequences may have a masking TβR sequence located at the C-terminus of a first scaffold polypeptide, and a TGF-β polypeptide (and optionally another MOD) located at the C-terminus of the second scaffold polypeptide sequence (see, e.g., FIGS. 1A and 1B). The masking TβR sequence may, for example, be a TβRII sequence lacking its N-terminal 25 aas (625) and bearing a D118A substitution: QLCKFCDVRF STCDNQKSCM SNCSITSICE KPQEVCVAVW RKNDENITLE TVCHDPKLPY HDFILEDAAS PKCIMKEKKK PGETFFMCSC SSAECNDNI IFSEEYNTSN PD (SEQ ID NO:172; see, also, SEQ ID NO:171). The TGF-β polypeptide may be a human TGF-β3 polypeptide bearing a C77S substitution: ALDTNYCFRN LEENCCVRPL YIDFRQDLGW KWVHEPKGYY ANFCSGPCPY LR SADTTHS TVLGLYNTLN PEASASPSCV PQDLEPLTIL YYVGRTPKVE QLSNMVVKSC KCS (SEQ ID NO:162). Linkers that are selected independently may be used to join the TGF-β and TβR sequences to the CIIC polypeptides (e.g., the scaffold polypeptide sequences as in FIG. 1, structures R-U and W).

### (2) IL-2 and its Variants

As one non-limiting example, a MOD or variant MOD present in a CIIC is an IL-2 or variant IL-2 polypeptide. Wild-type IL-2 binds to IL-2 receptor (IL-2R), which in some cases is a heterotrimeric polypeptide comprising an alpha chain (IL-2Ra, also referred to as CD25), a beta chain (IL-2Rp, also referred to as CD122) and a gamma chain (IL-2Ry, also referred to as CD132) (i.e., a heterotrimeric protein comprising IL-2Ra, IL-2Rp, and IL-2Ry). Amino acid sequences of human IL-2, human IL-2Ra, IL-2Rp, and IL-2Ry are known. See, e.g., published PCT applications WO2020/132138Δ1, WO2019/051091 and WO 2020/132297.

A wt. IL-2 MOD present in a CIIC may comprise at least 100, 110, 120, 130 or all 133 aas of the following IL-2 sequence: APTSSSTKKT QLQLEHLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (aa 21-153 of UniProt P60568, SEQ ID NO:208). An IL-2 MOD present in a CIIC may be a variant IL-2 polypeptide having at least 90% or at least 95% sequence identity to at least 110 contiguous aas of the IL-2 aa sequence of SEQ ID NO:208, and having one or more aa differences from the wt. IL-2 aa sequence. An IL-2 MOD present in a CIIC may be a variant IL-2 polypeptide having at least 98% or at least 99% sequence identity to at least 110 contiguous aas of the IL-2 aa sequence of SEQ ID NO:208, and having one or more aa differences from the wt. IL-2 aa sequence. An IL-2 MOD present in a CIIC of the present disclosure may be a variant IL-2 polypeptide having 100%, sequence identity to at least 120 contiguous aas of the IL-2 aa sequence of SEQ ID NO:208.

An IL-2 MOD present in a CIIC of the present disclosure may be a variant IL-2 polypeptide that exhibits decreased binding to IL-2Ra, thereby minimizing or substantially reducing the activation of T regs by the IL-2 variant. Alternatively, or additionally, in some cases, an IL-2 variant MOD of this disclosure exhibits decreased binding to IL-2R3 such that the IL-2 variant MOD exhibits an overall reduced affinity for IL-2R. In some cases, an IL-2 variant MOD of this disclosure exhibits both properties, i.e., it exhibits decreased or substantially no binding to IL-2Rα, and also exhibits decreased binding to IL-2Rβ such that the IL-2 variant polypeptide exhibits an overall reduced affinity for IL-2R. Such variants are disclosed in published PCT applications WO2020/132138Δ1, WO2019/051091 and WO2020/132297. Such variants also may exhibit decreased binding to IL-2Ry such that the IL-2 variant polypeptide exhibits an overall reduced affinity for IL-2R.

IL-2 variant MODs that exhibit decreased or substantially no binding to IL-2Rα, and also exhibit decreased binding to IL-2Rβ such that the IL-2 variant polypeptide exhibits an overall reduced affinity for IL-2R are disclosed in published PCT applications WO2020/132138Δ1, WO2019/051091 and WO2020/132297. For example, IL-2 variants having substitutions at H16 and F42 (e.g., the IL-2 variants in the 1715A polypeptide, shown in FIG. 2A, each of which have H16A and F42A substitutions) have shown decreased binding to IL-2Ra and IL-2Rp. See Quayle et al., Clin Cancer Res, 26(8) Apr. 15, 2020, which discloses that the binding affinity of an IL-2 polypeptide with H16A and F42A substitutions for human IL-2Ra and IL-2Rβ was decreased 110—and 3-fold, respectively, compared with wt. IL2 binding, predominantly due to a faster off-rate for each of these interactions. CIICs comprising such variants, including variants that exhibit decreased binding to IL-2Ra and IL-2Rp, have shown the ability to preferentially bind to and activate IL-2 receptors on T cells that contain the target TCR that is specific for the peptide epitope on the CIIC, and are thus less likely to deliver IL-2 to non-target T cells, i.e., T cells that do not contain a TCR that specifically binds the peptide epitope on the CIIC. That is, the binding of the IL-2 variant MOD to its co-MOD on the T cell is substantially driven by the binding of the MHC-epitope moiety rather than by the binding of the IL-2.

Suitable IL-2 variant MODs thus include a polypeptide that comprises an aa sequence having at least 90%, at least 95%, at least 98%, or at least 99% aa sequence identity to the wt. IL-2 aa sequence of SEQ ID NO:208, and having one or more amino acid differences from the wt. IL-2 aa sequence that cause the variant to exhibit decreased or substantially no binding to IL-2Rα, and also decreased binding to IL-2Rp.

In some cases, a suitable variant IL-2 polypeptide comprises an aa sequence having at least 90%, at least 95%, at least 98%, at least 99%, or 100% aa sequence identity to the aa sequence: APTSSSTKKT QLQLEALLLD LQMILNGINN YKNPKLTRML TAKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:209), i.e., the variant IL-2 polypeptide has the aa sequence of wt. IL-2, or with at least 95% identity to wt. IL-2, but with H16A and F42A substitutions (shown in bold and italics). Alternatively, the foregoing sequence, but with substitutions other than Ala at H16 and/or F42, may be employed, e.g., H16T, H16E or H16D may be employed instead of H16A.

Some exemplary combinations of mutations that reduce binding of an IL-2 variant polypeptide to IL-2Ra and IL-2Rβ include those listed in Table 2a (below).

TABLE 2a

| Mutation(s) to | Mutation(s) to | |
|---|---|---|
| decrease binding | decrease binding | Exemplary |
| to IL-2Rα | to IL-2Rβ | combinations |
| R38 with any amino | E15 with any amino | E15A with R38A, R38D or R38E |
| acid other than Arg, | acid other than Glu | |
| e.g., Ala, Asp, Glu | | |
| R38 with any amino | H16, with any amino | H16A with R38A, R38D or R38E |
| acid other than Arg, | acid other than His, | H16T with R38A, R38D or R38E |
| e.g., Ala, Asp, Glu | e.g., Ala, Glu, Thr, | H16E with R38A, R38D or R38E |
| | or Asp, | H16D with R38A, R38D or R38E |
| R38 with any amino | D84, with any amino | D84H with R38A, R38D or R38E |
| acid other than Arg, | acid other than Asp, | D84K with R38A, R38D or R38E |
| e.g., Ala, Asp, Glu | e.g., His, Lys or Arg | D84R with R38A, R38D or R38E |
| R38 with any amino | N88, with any amino | R38A with N88S, N88A, N88G, |
| acid other than Arg, | acid other than Asn, | N88R, N88T, or N88D |
| e.g., Ala, Asp, Glu | e.g., Ser, Ala, Gly, | R38D with N88S, N88A, N88G, |
| | Arg, Thr or Asp | N88R, N88T, or N88D |
| | | R38E with N88S, N88A, N88G, |
| | | N88R, N88T, or N88D |
| R38 with any amino | V91 with any amino | R38A with V91E, V91A or V91T |
| acid other than Arg, | acid other than Val, | R38D with V91E, V91A or V91T |
| e.g., Ala, Asp, Glu | e.g., Glu, Ala or Thr | R38E with V91E, V91A or V91T |
| R38 with any amino | I92 with any amino | R38A, I92A |
| acid other than Arg, | acid other than Ile, | R38D, I92A |
| e.g., Ala, Asp, Glu | e.g., Ala | R38E, I92A |
| F42, with any amino | E15 with any amino | E15A, F42A |
| acid other than Phe, | acid other than Glu | E15A, F42K |
| e.g., Ala or Lys, , as | | |
| well as Met, Pro, Ser, | | |
| Thr, Trp, Tyr, and Val | | |
| F42, with any amino | H16, with any amino | H16A, F42A; H16T, F42A; |
| acid other than Phe, | acid other than His, | H16E, F42A; H16D, F42A |
| e.g., Ala or Lys, as | e.g., Ala, Glu, Thr, | H16A, F42K; H16T, F42K; |
| well as Met, Pro, Ser, | or Asp, | H16E, F42K; H16D, F42K |
| Thr, Trp, Tyr, and Val | | |
| F42, with any amino | D84, with any amino | F42A with D84H, D84K or D84R |
| acid other than Phe, | acid other than Asp, | F42K with D84H, D84K or D84R |
| e.g., Ala or Lys, as | e.g., His, Lys or Arg | |
| well as Met, Pro, Ser, | | |
| Thr, Trp, Tyr, and Val | | |
| F42, with any amino | N88, with any amino | F42A with N88S, N88A, N88G, |
| acid other than Phe, | acid other than Asn, | N88R, N88T, or N88D |
| e.g., Ala or Lys, as | e.g., Ser, Ala, Gly, | F42K with N88S, N88A, N88G, |
| well as Met, Pro, Ser, | Arg, Thr or Asp | N88R, N88T, or N88D |
| Thr, Trp, Tyr, and Val | | |
| F42, with any amino | V91 with any amino | F42A with V91E, V91A, or V91T |
| acid other than Phe, | acid other than Val, | F42K with V91E, V91A, or V91T |
| e.g., Ala or Lys, as | e.g., Glu, Ala or Thr | |
| well as Met, Pro, Ser, | | |
| Thr, Trp, Tyr, and Val | | |
| F42, with any amino | I92 with any amino | F42A with I92A |
| acid other than Phe, | acid other than Ile, | F42K with I92A |
| e.g., Ala or Lys, as | e.g., Ala | |
| well as Met, Pro, Ser, | | |
| Thr, Trp, Tyr, and Val | | |
| K43, with any amino | E15 with any amino | E15A, K43E |
| acid other than Lys, | acid other than Glu | |
| e.g., Glu | | |
| K43, with any amino | H16, with any amino | H16A, K43E; H16T, K43E; |
| acid other than Lys, | acid other than His, | H16E, K43E; H16D, K43E |
| e.g., Glu | e.g., Ala, Glu, Thr, | |
| | or Asp, | |
| K43, with any amino | D84, with any amino | K43E with D84H, D84K or D84R |
| acid other than Lys, | acid other than Asp, | |
| e.g., Glu | e.g., His, Lys or Arg | |
| K43, with any amino | N88, with any amino | K43E with N88S, N88A, N88G, |
| acid other than Lys, | acid other than Asn, | N88R, N88T, or N88D |
| e.g., Glu | e.g., Ser, Ala, Gly, | |
| | Arg, Thr or Asp | |
| K43, with any amino | V91 with any amino | K43E with V91E, V91A, or V91T |
| acid other than Lys, | acid other than Val, | |
| e.g., Glu | e.g., Glu, Ala or Thr | |
| K43, with any amino | I92 with any amino | K43E, I92A |
| acid other than Lys, | acid other than Ile, | |
| e.g., Glu | e.g., Ala | |
| E62, with any amino | E15 with any amino | E15A, E62Q |
| acid other than Glu, | acid other than Glu | |
| e.g., Gln | | |
| E62, with any amino | H16, with any amino | H16A, E62Q; H16T, E62Q; |
| acid other than Glu, | acid other than His, | H16E, E62Q; H16D, E62Q |
| e.g., Gln | e.g., Ala, Glu, Thr, | |
| | or Asp, | |
| E62, with any amino | D84, with any amino | E62Q with D84H, D84K or D84R |
| acid other than Glu, | acid other than Asp, | |
| e.g., Gln | e.g., His, Lys or Arg | |
| E62, with any amino | N88, with any amino | E62Q with N88S, N88A, N88G, |
| acid other than Glu, | acid other than Asn, | N88R, N88T, or N88D |
| e.g., Gln | e.g., Ser, Ala, Gly, | |
| | Arg, Thr or Asp | |
| E62, with any amino | V91 with any amino | E62Q with V91E, V91A, or V91T |
| acid other than Glu, | acid other than Val, | |
| e.g., Gln | e.g., Glu, Ala or Thr | |
| E62, with any amino | I92 with any amino | E62Q, I92A |
| acid other than Glu, | acid other than Ile, | |
| e.g., Gln | e.g., Ala | |
| F42, with any amino | E15 with any amino | E15A, F42A with D84H, D84K or D84R |
| acid other than Phe, | acid other than Glu | E15A, F42K with D84H, D84K or D84R |
| e.g., Ala or Lys, as | D84, with any amino | |
| well as Met, Pro, Ser, | acid other than Asp, | |
| Thr, Trp, Tyr, and Val | e.g., His, Lys and Arg | |
| F42, with any amino | E15 with any amino | E15A, F42A with N88S, N88A, |
| acid other than Phe, | acid other than Glu | N88G, N88R, N88T, or N88D |
| e.g., Ala or Lys, as | N88, with any amino | E15A, F42K with N88S, N88A, |
| well as Met, Pro, Ser, | acid other than Asn, | N88G, N88R, N88T, or N88D |
| Thr, Trp, Tyr, and Val | e.g., Ser, Ala, Gly, | |
| | Arg, Thr, and Asp | |
| F42, with any amino | E15 with any amino | E15A, F42A with V91E, V91A, or V91T |
| acid other than Phe, | acid other than Glu | E15A, F42K with V91E, V91A, or V91T |
| e.g., Ala or Lys, as | V91 with any amino | |
| well as Met, Pro, Ser, | acid other than Val, | |
| Thr, Trp, Tyr, and Val | e.g., Glu, Ala or Thr | |
| F42, with any amino | E15 with any amino | E15A, F42A with I92A |
| acid other than Phe, | acid other than Glu | E15A, F42K with I92A |
| e.g., Ala or Lys, as | I92 with any amino | |
| well as Met, Pro, Ser, | acid other than Ile, | |
| Thr, Trp, Tyr, and Val | e.g., Ala | |
| F42, with any amino | H16, with any amino | H16A, F42A with D84H, D84K or D84R |
| acid other than Phe, | acid other than His, | H16A, F42K with D84H, D84K or D84R |
| e.g., Ala or Lys, as | e.g., Ala, Glu, Thr, | H16T, F42A with D84H, D84K or D84R |
| well as Met, Pro, Ser, | or Asp | H16T, F42K with D84H, D84K or D84R |
| Thr, Trp, Tyr, and Val | D84, with any amino | H16E, F42A with D84H, D84K or D84R |
| | acid other than Asp, | H16E, F42K with D84H, D84K or D84R |
| | e.g., His, Lys and Arg | H16D, F42A with D84H, D84K or D84R |
| | | H16D, F42K with D84H, D84K or D84R |
| F42, with any amino | H16, with any amino | H16A, F42A with N88S, N88A, |
| acid other than Phe, | acid other than His, | N88G, N88R, N88T, or N88D |
| e.g., Ala or Lys, as | e.g., Ala, Glu, Thr, | H16A, F42K with N88S, N88A, |
| well as Met, Pro, Ser, | or Asp | N88G, N88R, N88T, or N88D |
| Thr, Trp, Tyr, and Val | N88, with any amino | H16T, F42A with N88S, N88A, |
| | acid other than Asn, | N88G, N88R, N88T, or N88D |
| | e.g., Ser, Ala, Gly, | H16T, F42K with N88S, N88A, |
| | Arg, Thr, and Asp | N88G, N88R, N88T, or N88D |
| | | H16E, F42A with N88S, N88A, |
| | | N88G, N88R, N88T, or N88D |
| | | H16E, F42K with N88S, N88A, |
| | | N88G, N88R, N88T, or N88D |
| | | H16D, F42A with N88S, N88A, |
| | | N88G, N88R, N88T, or N88D |
| | | H16D, F42K with N88S, N88A, |
| | | N88G, N88R, N88T, or N88D |
| F42, with any amino | H16, with any amino | H16A, F42A with V91E, V91A, or V91T |
| acid other than Phe, | acid other than His, | H16A, F42K with V91E, V91A, or V91T |
| e.g., Ala or Lys, as | e.g., Ala, Glu, Thr, | H16T, F42A with V91E, V91A, or V91T |
| well as Met, Pro, Ser, | or Asp | H16T, F42K with V91E, V91A, or V91T |
| Thr, Trp, Tyr, and Val | V91 with any amino | H16E, F42A with V91E, V91A, or V91T |
| | acid other than Val, | H16E, F42K with V91E, V91A, or V91T |
| | e.g., Glu, Ala or Thr | H16D, F42A with V91E, V91A, or V91T |
| | | H16D, F42K with V91E, V91A, or V91T |
| F42, with any amino | H16, with any amino | H16A, F42A with I92A |
| acid other than Phe, | acid other than His, | H16A, F42K with I92A |
| e.g., Ala or Lys, as | e.g., Ala, Glu, Thr, | H16T, F42A with I92A |
| well as Met, Pro, Ser, | or Asp | H16T, F42K with I92A |
| Thr, Trp, Tyr, and Val | I92 with any amino | H16E, F42A with I92A |
| | acid other than Ile, | H16E, F42K with I92A |
| | e.g., Ala | H16D, F42A with I92A |
| | | H16D, F42K with I92A |

A CIIC may comprise two copies of a wt. and/or a variant IL-2 polypeptide located in tandem, where they are linked together by a peptide linker.

In any of the wt. or variant IL-2 sequences provided herein, the cysteine at position 125 of the wt. sequence provided in SEQ ID NO:208 may be substituted with an aa other than cysteine, such as alanine (a C125A substitution). In addition to any stability provided by the substitution, it may be employed where, for example, an additional peptide is to be conjugated to a cysteine residue elsewhere in a CIIC, thereby avoiding competition from the C125 of the IL-2 MOD sequence.

Alternatively, because binding to IL-2Ra can lead to an increase in T regs, it may be desirable to employ an IL-2 variant that does not have substantially decreased binding to IL-2Ra as compared to wt. IL-2 or which possesses increased binding to IL-2Ra as compared to wt. IL-2. Accordingly, in some instances, a CIIC can comprise one or more IL-2 variant MODs that do not have substantially decreased binding to IL-2Ra as compared to wt. IL-2 or which possess increased binding to IL-2Ra as compared to wt. IL-2. Such MODs also may have decreased binding to IL-2R13 as compared to wt. IL-2, e.g., by a substitution of H16 such as H16A or H16T.

### (3) Fas Ligand (FasL) and its Variants

In some cases, a wt. and/or a variant Fas ligand (FasL) polypeptide sequence is present as a MOD in a CIIC. FasL is a homomeric type-II transmembrane protein in the tumor necrosis factor (TNF) family. FasL signals by trimerization of the Fas receptor in a target cell, which forms a death-inducing complex leading to apoptosis of the target cell. Soluble FasL results from matrix metalloproteinase-7 (MMP-7) cleavage of membrane-bound FasL at a conserved site.

In an embodiment, a wt. Homo sapiens FasL protein has the sequence: MQQPFNYPYP QIYWVDSSAS SPWAPPGTVL PCPTSVPRRP GQRRPPPPPP PPPLPPPPPP PPLPPLPLPP LKKRGNHSTG LCLLVMFFMV LVALVGLGLG MFQLFHLQKE LAELRESTSQ MHTASSLEKQ IGHPSPPPEK KELRKVAHLT GKSNSRSMPL EWEDTYGIVL LSGVKYKKGG LVINETGLYF VYSKVYFRGQ SCNNLPLSHK VYMRNSKYPQ DLVMMEGKMM SYCTTGQMWA RSSYLGAVFN LTSADHLYVN VSELSLVNFE ESQTFFGLYK L (SEQ ID NO:372), NCBI Ref. Seq. NP_000630.1, UniProtKB—P48023 where aas 1-80 are cytoplasmic, aas 81-102 are the transmembrane domain and aas 103-281 are extracellular (ectodomain). In some cases, a FasL polypeptide suitable for inclusion in a CIIC comprises an aa sequence having at least 85%, at least 90%, at least 95%, at least 98%, at least 99%, or 100% aa sequence identity to a contiguous stretch of at least 150 aas, at least 170 aas, at least 180 aas, at least 200 aas, at least 225 aas, at least 250 aas, at least 270 aas, at least 280 aas, or all aas of the aa sequence of SEQ ID NO:372).

A Fas receptor can have the sequence: MLGIWTLLPL VLTSVARLSS KSVNAQVTDI NSKGLELRKT VTTVETQNLE GLHHDGQFCH KPCPPGERKA RDCTVNGDEP DCVPCQEGKE YTDKAHFSSK CRRCRLCDEG HGLEVEINCT RTQNTKCRCK PNFFCNSTVC EHCDPCTKCE HGIIKECTLT SNTKCKEEGS RSNLGWLCLL LLPIPLIVWV KRKEVQKTCR KHRKENQGSH ESPTLNPETV AlNLSDVDLS KYITTIAGVM TLSQVKGFVR KNGVNEAKID EIKNDNVQDT AEQKVQLLRN WHQLHGKKEA YDTLIKDLKK ANLCTLAEKI QTIILKDITS DSENSNFRNE IQSLV (SEQ ID NO:373), NCBI Reference Sequence: NP_000034.1, UniProtKB—P25445, where aas 26-173 form the ectodomain (extracellular domain), aas 174-190 form the transmembrane domain, and aas 191-335 form the cytoplasmic domain. The ectodomain may be used to determine binding affinity with FasL.

A FasL polypeptide suitable for inclusion in a CIIC may comprise an aa sequence having at least 85%, at least 90%, at least 95%, at least 98%, at least 99%, or 100% aa sequence identity to the following aa sequence: IGHPSPPPEK KELRKVAHLT GKSNSRSMPL EWEDTYGIVL LSGVKYKKGG LVINETGLYF VYSKVYFRGQ SCNNLPLSHK VYMRNSKYPQ DLVMMEGKMMSYCTTGQMWA RSSYLGAVFN LTSADHLYVN VSELSLVNFE ESQTFFGLYK (SEQ ID NO:374/), and has a length of about 150 aas, including 148, 149, 150, 151, or 152 aas.

In some cases, a FasL polypeptide suitable for inclusion in a CIIC comprises an aa sequence having at least 85%, at least 90%, at least 95%, at least 98%, at least 99%, or 100% aa sequence identity to a contiguous stretch of at least 150 aas, at least 160 aas, at least 170 aas, at least 175 aas, or all of the aas of the following aa sequence: QLFHLQKE LAELRESTSQ MHTASSLEKQ IGHPSPPPEK KELRKVAHLT GKSNSRSMPL EWEDTYGIVL LSGVKYKKGG LVINETGLYF VYSKVYFRGQ SCNNLPLSHK VYMRNSKYPQ DLVMMEGKMM SYCTTGQMWA RSSYLGAVFN LTSADHLYVN VSELSLVNFE ESQTFFGLYK L (SEQ ID NO:375). Suitable variant FasL polypeptide sequences include polypeptide sequences with at least 85%, at least 90%, at least 95%, at least 98%, or at least 99% aa sequence identity to at least 140 contiguous aas (e.g., at least 150, at least 160, at least 170, or at least 175 contiguous aas) of SEQ ID NO:375) (e.g., which have at least one aa substitution, deletion or insertion).

In some cases, a variant FasL polypeptide (e.g., comprising a variant of SEQ ID NO:374 or SEQ ID NO:375) exhibits reduced binding affinity to a mature Fas receptor sequence (e.g., a FasL receptor comprising all or part of the polypeptide set forth in SEQ ID NO:373, such as its ectodomain), compared to the binding affinity of a FasL polypeptide comprising the aa sequence set forth in SEQ ID NO:374 or SEQ ID NO:375. For example, in some cases, a variant FasL polypeptide (e.g., comprising a variant of SEQ ID NO:375) binds a Fas receptor (e.g., comprising all or part of the polypeptides set forth in SEQ ID NO:373, such as its ectodomain) with a binding affinity that is at least 10% less, at least 20% less, at least 30% less, at least 40% less, at least 50% less, at least 60% less, at least 70% less, at least 80% less, at least 90% less, at least 95% less, or more than 95% less than the binding affinity of a FasL polypeptide comprising the aa sequence set forth in SEQ ID NO:372 or SEQ ID NO:375.

### (4) PD-L1 and its Variants

As one non-limiting example, a MOD or variant MOD present in a CIIC is a PD-L1 or variant PD-L1 polypeptide. Wild-type PD-L1 binds to PD1.

A wt. human PD-L1 polypeptide can comprise the following aa sequence: MRIFAVFIFM TYWHLLNAFT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKICLT LSPST (SEQ ID NO:210), where aas 1-18 form the signal sequence, aas 19-127 form the Ig-like V-type or “IgV” domain, and aas 133-225 form the Ig-like C2 type domain.

A wt. human PD-L1 ectodomain aa sequence can comprise the following aa sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:211), where aas 1-109 form the Ig-like V-type or “IgV” domain, and aas 115-207 form the Ig-like C2 type domain.

A wt. human PD-L1 ectodomain aa sequence can also comprise the following aa sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPELP LAHPPNER LNVSIKI (SEQ ID NO:212); where aas 1-109 form the Ig-like V-type or “IgV” domain, and aas 115-207 form the Ig-like C2 type domain. See, e.g., NCBI Accession and version 3BIK_A, which includes an N-terminal alanine as its first aa.

A wt. PD-L1 IgV domain, suitable for use as a MOD may comprise aa 18 and aas IgV aas 19-127 (i.e., aas 18-127) of SEQ ID NO:210, and a carboxyl terminal stabilization sequence, such as for instance the last seven aas (bolded and italicized) of the sequence: A FTVTVPKDLY VVEYGSNMTI ECKFPVEKQL DLAALIVYWE MEDKNIIQFV HGEEDLKTQH SSYRQRARLL KDQLSLGNAA LQITDVKLQD AGVYRCMISY GGADYKRITV KVNAPYAAAL HEH (SEQ ID NO:213). Where the carboxyl stabilizing sequence comprises a histidine (e.g., a histidine approximately 5 residues to the C-terminal side of the Tyr (Y) appearing as aa 117 of SEQ ID NO:213) (aa 118 to about aa 122), the histidine may form a stabilizing electrostatic bond with the backbone amide at aas 82 and 83 (bolded and italicized in SEQ ID NO:210 (Q107 and L106 of SEQ ID NO:210). As an alternative, a stabilizing disulfide bond may be formed by substituting one of aas 82 or 83) (Q107 and L106 of SEQ ID NO:210) and one of aa residues 121, 122, or 123 (equivalent to aa positions 139-141 of SEQ ID NO:210).

A wt. PD-1 polypeptide can comprise the following aa sequence: PGWFLDSPDR PWNPPTFSPA LLVVTEGDNA TFTCSFSNTS ESFVLNWYRM SPSNQTDKLA AFPEDRSQPG QDCRFRVTQL PNGRDFHMSV VRARRNDSGT YLCGAISLAP KAQIKESLRA ELRVTERRAE VPTAHPSPSP RPAGQFQTLV VGVVGGLLGS LVLLVWVLAV ICSRAARGTI GARRTGQPLK EDPSAVPVFS VDYGELDFQW REKTPEPPVP CVPEQTEYAT IVFPSGMGTS SPARRGSADG PRSAQPLRPE DGHCSWPL (SEQ ID NO:214).

In some cases, a variant PD-L1 polypeptide (e.g., a variant of SEQ ID NO:211 or PD-L1's IgV domain) exhibits reduced binding affinity to PD-1 (e.g., a PD-1 polypeptide comprising the aa sequence set forth in SEQ ID NO:214), compared to the binding affinity of a PD-L1 polypeptide comprising the aa sequence set forth in SEQ ID NO:210 or SEQ ID NO:211. For example, in some cases, a variant PD-L1 polypeptide binds PD-1 (e.g., a PD-1 polypeptide comprising the aa sequence set forth in SEQ ID NO:214) with a binding affinity that is at least 10% less, at least 20% less, at least 30% less, at least 40% less, at least 50% less, at least 60% less, at least 70% less, at least 80% less, at least 90% less, at least 95% less, or more than 95% less than the binding affinity of a PD-L1 polypeptide comprising the aa sequence set forth in SEQ ID NO:210 or SEQ ID NO:211.

Suitable PD-L1 polypeptide aa sequences (wt. and variant) for inclusion in a MAPP may comprise polypeptide sequences with at least 85%, at least 90%, at least 95%, at least 98%, or at least 99% aa sequence identity to a PD-L1 sequence of SEQ ID NO:211, SEQ ID NO:212, SEQ ID NO:213, or aas 18-127 of SEQ ID NO:210. In an embodiment, a PD-L1 MOD aa sequence comprises a polypeptide sequence with at least 90% or at least 95% aa sequence identity to a PD-L1 sequence of SEQ ID NO:211, SEQ ID NO:212, SEQ ID NO:213, or aas 18-127 of SEQ ID NO:210.

### (5) IL-10 and its Variants

In some cases, a wt. and/or a variant IL-10 MOD sequence is present as a MOD in a CIIC. Wt. IL-10 binds to the IL-10 receptor, which is composed of two subunits, IL-10RI a ligand-binding subunit, and IL-10RII an accessory subunit required for signal transduction.

A wt. IL-10 aa sequence can be as follows: SPGQGTQSEN SCTHFPGNLP NMLRDLRDAF SRVKTFFQMK DQLDNLLLKE SLLEDFKGYL GCQALSEMIQ FYLEEVMPQA ENQDPDIKAH VNSLGENLKT LRLRLRRCHR FLPCENKSKAVEQVKNAFNK LQEKGIYKAM SEFDIFINYI EAYMTMKIRN (SEQ ID NO:215). See, e.g., NCBI Reference Sequence: NP_000563.1.

IL-10 polypeptides suitable for use as a MOD in a CIIC include those polypeptides comprising the sequence provided in SEQ ID NO:215. IL-10 polypeptides suitable for inclusion in a CIIC also include polypeptides having at least 90% or at least 95% aa sequence identity to at least 140 contiguous aas of SEQ ID NO:215 and at least one aa substitution, deletion, and/or insertion. IL-10 polypeptides suitable for inclusion in a CIIC also include polypeptides having at least 97% or at least 99% aa sequence identity to at least 140 contiguous aas of SEQ ID NO:215 and at least one aa substitution, deletion, or insertion. Examples of such IL-10 peptides include those having at least two, three, or four aa substitutions, insertions and/or deletions.

IL-10 polypeptides suitable for inclusion in a CIIC also include those with conserved N-terminal and/or C-terminal regions that have been shown to be involved in different functions of IL-10. See, e.g., Gesser et al. Proc. Natl. Acad. Sci. USA, 94, 14620-14625 (1997). The conserved N-terminal sequence is reported to be associated with (i) inhibition of IL-1b-induced IL-8 production by peripheral blood mononuclear cells, (ii) inhibition of spontaneous IL-8 production and induction of IL-1 receptor antagonistic protein production by human monocytes, (iii) induction of chemotactic migration in vitro and desensitization of human CD81 T cells resulting in an unresponsiveness toward rhIL-10-induced chemotaxis, (iv) suppression of the chemotactic response to IL-8 and induction of IL-4 production by cultured normal human CD41 T cells, (v) down-regulation of TNF-α production by CD81 T cells, and (vi) inhibition of class II MHC antigen expression on IFN-γ stimulated human monocytes. The conserved C-terminal region is reported to be a regulator of mast cell proliferation.

In view of the foregoing, IL-10 polypeptides with a conserved N-terminal region suitable for inclusion in a CIIC include polypeptides having at least 90% or at least 95% sequence identity to the polypeptide of SEQ ID NO:215 and having an N-terminal sequence comprising the nonapeptide, SPGQGTQSE (SEQ ID NO:216), or a sequence with up to 1 aa substitution, deletion and/or insertion in that nonapeptide. IL-10 polypeptides with a conserved N-terminal region suitable for inclusion in a CIIC also include polypeptides having at least 97% or at least 98% sequence identity to at least 140 contiguous aas of SEQ ID NO:215, and having an N-terminal sequence comprising the nonapeptide, SPGQGTQSE (SEQ ID NO:216), or a sequence with up to 1 aa substitution, deletion and/or insertion in that nonapeptide. IL-10 polypeptides suitable for inclusion in a CIIC include polypeptides with a conserved C-terminal region having at least 90% or at least 95% sequence identity to the polypeptide of SEQ ID NO:215 and having a C-terminal sequence comprising the nonapeptide, AYMTMKIRN (SEQ ID NO:217), or a sequence with up to 1 aa substitution, deletion and/or insertion in that nonapeptide. IL-10 polypeptides with a conserved C-terminal region suitable for inclusion in a CIIC also include polypeptides having at least 97% or at least 98% sequence identity to at least 140 contiguous aas of SEQ ID NO:215, and having a C-terminal sequence comprising the nonapeptide, AYMTMKIRN (SEQ ID NO:217), or a sequence with up to 1 aa substitution, deletion and/or insertion in that nonapeptide. An IL-10 polypeptide suitable for inclusion in a CIIC also includes a polypeptide having at least 95% or at least 98% sequence identity to at least 140 contiguous aas of SEQ ID NO:215, and having both an N-terminal sequence comprising the nonapeptide, SPGQGTQSE (SEQ ID NO:216), and a C-terminal sequence comprising the nonapeptide, AYMTMKIRN (SEQ ID NO:217), with up to 3 aa substitutions, deletions and/or insertions in either or both of those nonapeptides.

IL-10 polypeptides suitable for inclusion in a CIIC also include polypeptides having at least 90% or at least 95% aa sequence identity to at least 140 contiguous aas of the sequence, SPGQGTQSEN SCTHFPGNLP NMLRX1LRDAF SRVKTFFQMK DQLDNLLLKE SLLEDFKGYL GCQALSEMIQ FYLEEVMPQA ENQDPDIKAH VNSLGX2NLKT LRLRLRRCHR FLPCENKSKAVEQVKNAFNK LQEKGIYKAM SEFDIFINYI EAYMTMKIRN (SEQ ID NO:218), wherein X1 is other than D and/or X2 is other than E. For example, X1 and X2 may be substituted by an A (D25A, E97A substitutions): SPGQGTQSEN SCTHFPGNLP NMLRALRDAF SRVKTFFQMK DQLDNLLLKE SLLEDFKGYL GCQALSEMIQ FYLEEVMPQA ENQDPDIKAH VNSLGANLKT LRLRLRRCHR FLPCENKSKA VEQVKNAFNK LQEKGIYKAM SEFDIFINYI EAYMTMKIRN (SEQ ID NO:219).

### (6) CD80 and its Variants

In some cases, a wt. and/or a variant CD80 MOD sequence is present as a MOD in a CIIC. Wt. CD80 and variant CD80 MODs bind to CD28 which acts as their receptor.

A wt. aa sequence of the ectodomain of human CD80 can be as follows: VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:376). See NCBI Reference Sequence: NP_005182.1. The aa sequence of the IgV domain of a wt. human CD80 can be as follows: VIHVTK EVKEVATLSC GHNVSVEELA QTRIYWQKEK KMVLTMMSGD MNIWPEYKNR TIFDITNNLS IVILALRPSD EGTYECWLK YEKDAFKREH LAEVTLSV (SEQ ID NO:377), which is aas 1-104 of SEQ ID NO:376.

A wt. CD28 aa sequence can be as follows: MLRLLLALNL FPSIQVTGNK ILVKQSPMLV AYDNAVNLSC KYSYNLFSRE FRASLHKGLD SAVEVCVVYG NYSQQLQVYS KTGFNCDGKL GNESVTFYLQ NLYVNQTDIY FCKIEVMYPP PYLDNEKSNG TIIHVKGKHL CPSPLFPGPS KPFWVLVVVG GVLACYSLLV TVAFIIFWVR SKRSRLLHSD YMNMTPRRPG PTRKHYQPYA PPRDFAAYRS (SEQ ID NO:378).

A wt. CD28 aa sequence can be as follows: MLRLLLALNL FPSIQVTGNK ILVKQSPMLV AYDNAVNLSW KHLCPSPLFP GPSKPFWVLV VVGGVLACYS LLVTVAFIIF WVRSKRSRLL HSDYMNMTPR RPGPTRKHYQ PYAPPRDFAA YRS (SEQ ID NO:379)

A wt. CD28 aa sequence can be as follows: MLRLLLALNL FPSIQVTGKH LCPSPLFPGP SKPFWVLVVV GGVLACYSLL VTVAFIIFWV RSKRSRLLHS DYMNMTPRRP GPTRKHYQPY APPRDFAAYR S (SEQ ID NO:380).

Variant CD80 polypeptides suitable as a MOD in a CIIC of the present disclosure may exhibit reduced binding affinity to CD28, compared to the binding affinity of a CD80 polypeptide comprising the aa sequence set forth in SEQ ID NO:376, or the IgV domain sequence SEQ ID NO:378, for CD28. A variant CD80 MOD may bind CD28 with a binding affinity that is at least 10% less, at least 20% less, at least 30% less, at least 40% less, at least 50% less, at least 60% less, at least 70% less, at least 80% less, at least 90% less, at least 95% less, or more than 95% less than the binding affinity of a CD80 polypeptide comprising the aa sequence set forth in SEQ ID NO:376 for CD28 (e.g., a CD28 polypeptide comprising the aa sequence set forth in one of SEQ ID NO:378, SEQ ID NO:379, or SEQ ID NO:380).

CD80 ectodomain variants suitable for use as a MOD in a CIIC include those polypeptides with at least one aa substitution having at least 90%, at least 95%, at least 98%, or at least 99% aa sequence identity to SEQ ID NO:376, or the IgV domain sequence SEQ ID NO:378.

CD80 ectodomain variants suitable for use as a MOD in a CIIC include those polypeptides having at least 90%, at least 95%, at least 98%, or at least 99% aa sequence identity to SEQ ID NO:376, or the IgV domain sequence SEQ ID NO:378, and having at least one (e.g., at least two, or at least three) aa substitutions.

CD80 ectodomain variants suitable for use as a MOD in a CIIC include those polypeptides having at least 90% (e.g., at least 95%, 98%, or 99%) aa sequence identity to at least 80 (e.g., at least 90, 100, 104, 120, 150, 180, 200, or 208) contiguous aas of SEQ ID NO:376, or least 80 (e.g., at least 90, 100, or 104) contiguous aas of the IgV domain sequence of SEQ ID NO:378.

### (7) CD86 and its Variants

In some cases, a wt. and/or a variant CD86 MOD sequence is present as a MOD in a CIIC. Wt. CD86 and variant CD86 MODs bind to CD28 which acts as their receptor as discussed for CD80 MODs.

A wt. aa sequence of the ectodomain of human CD86 can be as follows: APLKIQAYFN ETADLPCQFA NSQNQSLSEL VVFWQDQENL VLNEVYLGKE KFDSVHSKYM NRTSFDSDSW TLRLHNLQIK DKGLYQCIIH HKKPTGMIRI HQMNSELSVL ANFSQPEIVP ISNITENVYI NLTCSSIHGY PEPKKMSVLL RTKNSTIEYD GIMQKSQDNV TELYDVSISL SVSFPDVTSN MTIFCILETD KTRLLSSPFS IELEDPQPPP DHIP (SEQ ID NO:381).

The aa sequence of the IgV domain of a wt. human CD86 can be as follows: APLKIQAYFN ETADLPCQFA NSQNQSLSEL VVFWQDQENL VLNEVYLGKE KFDSVHSKYM NRTSFDSDSW TLRLHNLQIK DKGLYQCIIH HKKPTGMIRI HQMNSELSVL (SEQ ID NO:382).

Variant CD86 polypeptides suitable as a MOD in a CIIC may exhibit reduced binding affinity to CD28, compared to the binding affinity of a CD86 polypeptide comprising the aa sequence set forth in SEQ ID NO:381 or SEQ ID NO:382 for CD28. A variant CD86 MOD may bind CD28 with a binding affinity that is at least 10% less, at least 20% less, at least 30% less, at least 40% less, at least 50% less, at least 60% less, at least 70% less, at least 80% less, at least 90% less, at least 95% less, or more than 95% less than the binding affinity of a CD86 polypeptide comprising the aa sequence set forth in SEQ ID NO:381 or SEQ ID NO:382 for CD28 (e.g., a CD28 polypeptide comprising the aa sequence set forth in one of SEQ ID NO:378, SEQ ID NO:379, or SEQ ID NO:380).

CD86 ectodomain variants suitable for use as a MOD in a CIIC include those polypeptides with at least one aa substitution having at least 90%, at least 95%, at least 98%, or at least 99% aa sequence identity to SEQ ID NO:381, or the IgV domain sequence SEQ ID NO:382.

CD86 ectodomain variants suitable for use as a MOD in a CIIC include those polypeptides having at least 90%, at least 95%, at least 98%, or at least 99% aa sequence identity to SEQ ID NO:381, or the IgV domain sequence SEQ ID NO:382, and having at least one (e.g., at least two, or at least three) aa substitution.

CD86 ectodomain variants suitable for use as a MOD in a CIIC include those polypeptides with at least one aa substitution having at least 90%, at least 95%, at least 98%, or at least 99% aa sequence identity to at least 80 (e.g., at least 90, 100, 109, 120, 150, 180, 200, or 224) contiguous aas of SEQ ID NO:381, or at least 80 (e.g., at least 90, 100, or 104) contiguous aas of the IgV domain sequence SEQ ID NO:382.

### 3. Scaffold

Scaffold polypeptide sequences serve, among other things, as structural elements upon which CIIC components can be built (see, e.g., FIG. 1, structure A, with a scaffold). Where a scaffold polypeptide is present in the CIIC it is generally located C-terminal to the MHC α subunit α2 domain, and may be connected to the α2 domain directly, or indirectly via an L3 linker sequence (see FIG. 1, structure A). Where a scaffold polypeptide sequence is present the CIIC may be considered a fusion protein comprising the scaffold as one of the fused protein elements. Depending on the nature of the scaffold, it can also act as an organizational element providing higher order CIIC complexes. Where one or more aa sequences present in a scaffold polypeptide permit the scaffold polypeptide to interact (specifically bind) with the scaffold polypeptide sequence of at least one other CIIC, the CIIC constructs can form higher order structures. For example, CIICs may be organized into, e.g., dimers (e.g., form homoduplexes or heteroduplexes), trimers or “triplexes,” tetramers or “quadraplexes,” pentamers or “pentaplexes” etc.). This is exemplified by the homoduplexes shown in FIG. 1 as structures H, I, and O where the scaffold polypeptide sequences may be capable of dimerizing, such as the case with some Ig Fc domains. Other multimerizing immunoglobulin scaffold sequences may be utilized including scaffold polypeptide sequences comprising IgM Fc regions (see, e.g., SEQ ID NO:13) that permit formation of pentameric CIICs (particularly when j-chain sequences are also expressed, e.g., SEQ ID NO:15) or hexameric CIICs. Petrušić et al., Med Hypotheses. 77(6):959-61 (2011).

Suitable scaffold polypeptides will, in some cases, be half-life extending polypeptides. In some cases, a suitable scaffold polypeptide (e.g., an immunoglobulin Fc sequence) increases the in vivo half-life (e.g., the circulating serum half-life) of a CIIC, compared to a control CIIC either lacking the scaffold polypeptide or having a scaffold polypeptide with a different (e.g., non-immunoglobulin Fc) scaffold sequence, by at least about 10%, at least about 15%, at least about 25%, at least about 50%, at least about 2-fold, at least about 5-fold, at least about 10-fold, at least about 25-fold, at least about 50-fold, at least about 100-fold, or more than 100-fold. As an example, in some cases an Ig Fc polypeptide scaffold sequence increases the stability and/or in vivo half-life (e.g., the serum half-life) of a CIIC, compared to a control CIIC either lacking the Ig Fc or having the Ig Fc polypeptide sequence replaced by a linker (e.g., a GGGGS (SEQ ID NO:237) repeat of equal sequence length). The increase in in vivo half-life can be at least about 10%, at least about 15%, at least about 25%, at least about 50%, at least about 2-fold, at least about 2.5-fold, at least about 5-fold, at least about 10-fold, at least about 25-fold, at least about 50-fold, at least about 100-fold, or more than 100-fold.

Scaffold polypeptide sequences generally may be less than about 300 aa (e.g., about 100 to about 300 aa). Scaffold polypeptide sequences may be less than about 250 aa (e.g., about 150 to about 250 aa). Scaffold polypeptide sequences may be less than about 200 aa (e.g., about 100 to about 200 aa). Scaffold polypeptide sequences may be less than about 150 aa (e.g., about 50 to about 150 aa). Alternatively, scaffold polypeptides may be greater than about 300 aa. For example, a scaffold polypeptide may be greater than about 300 and less than about 500 aas, greater than about 500 aas and less than about 600 aas, or greater than about 600 aas. For instance, a scaffold peptide sequence may be a serum albumin (e.g., human serum albumin) polypeptide sequence comprising most or all of the albumin protein.

Scaffold and other polypeptide sequences include interspecific and non-interspecific polypeptide sequences. Interspecific binding sequences are non-identical polypeptide sequences that selectively interact with their specific complementary counterpart sequence to form asymmetric pairs (heterodimers). Accordingly, interspecific sequences result substantially or completely in the formation of heteroduplexes (heterodimers), but may in some instances form some amount of homodimers, even though interspecific binding sequences can preferentially dimerize (by binding more strongly) with their counterpart interspecific binding sequence. By way of example, where an interspecific binding sequence and its counterpart are incorporated into a pair of polypeptides, they may selectively form greater than 70%, 80%, 90%, 95%, 98% or 99% heterodimers when an approximately equimolar mixture of the polypeptides are combined (co-expressed). The remainder of the polypeptides may be present as monomers or homodimers, which may be separated from the heterodimer. Because interspecific sequences are selective for their counterpart sequence, they can limit the interaction with other proteins expressed by cells (e.g., in culture or in a subject) particularly where the interspecific sequences are not naturally occurring or are variants of naturally occurring protein sequences. Interspecific scaffold sequence find use, for example, where it is desirable for a CIIC to present different MODs on a first and second CIIC polypeptide (see, e.g., FIG. 1, structure W) or where it is desirable to present a targeting sequence on a first CIIC and one or more (e.g., two or more) MODs on a second CIIC (see e.g., structure X). It is also possible to utilize interspecific scaffolds to provide a membrane anchored duplex CIIC by providing a MAS (e.g., transmembrane domain) on one CIIC of the interspecific duplex and MODs on the CIIC bearing the interspecific counterpart sequence (e.g., replacing the targeting sequence of FIG. 1, structure X with a transmembrane domain).

In contrast to interspecific sequences, non-interspecific sequences do not require specific non-identical sequences to dimerize and produce substantially or completely homodimers as shown in FIG. 1, structures H and I. Where both polypeptides of a CIIC duplex are to present otherwise identical CIIC component sequences, either an interspecific or non-interspecific scaffold sequence may be employed; however, the use of interspecific scaffold sequences in such a case would require a double transformation of the cell expressing CIIC duplex. In those instances where non-identical first and second polypeptides (e.g., CIIC polypeptides) containing non-interspecific binding sequences capable of interaction are produced in the same system, a population of molecules comprising homodimers of the first polypeptide, homodimers of the second polypeptide, and heterodimers of the first and second polypeptides can be formed.

### a) Non-Immunoglobulin Fc Scaffold Polypeptides

Non-immunoglobulin Fc scaffold polypeptides include, but are not limited to: albumin, XTEN (extended recombinant); transferrin; Fc receptor, elastin-like; albumin-binding; silk-like (see, e.g., Valluzzi et al. (2002) Philos Trans R Soc Lond B Biol Sci. 357:165); silk-elastin-like (SELP; see, e.g., Megeed et al. (2002) Adv Drug Deliv Rev. 54:1075) polypeptides; and the like. Suitable XTEN polypeptides include, e.g., those disclosed in WO 2009/023270, WO 2010/091122, WO 2007/103515, US 2010/0189682, and US 2009/0092582; see, also, Schellenberger et al. (2009) Nat Biotechnol. 27:1186). Suitable albumin polypeptides include, e.g., human serum albumin. Suitable elastin-like polypeptides are described, for example, in Hassouneh et al. (2012) Methods Enzymol. 502:215.

Other non-immunoglobulin Fc scaffold polypeptide sequences include but are not limited to: polypeptides of the collectin family (e.g., ACRP30 or ACRP30-like proteins) that contain collagen domains consisting of collagen repeats Gly-Xaa-Yaa and/or Gly-Xaa-Pro (which may be repeated from 10-40 times); coiled-coil domains; leucine-zipper domains; Fos/Jun binding pairs; and Ig CH1 and light chain constant region C_{L} sequences (Ig CH1/C_{L} pairs such as an Ig CH1 sequence paired with an Ig C_{L} K or C_{L} A light chain constant region sequence).

Non-immunoglobulin Fc scaffold polypeptides can be interspecific or non-interspecific in nature. For example, both Fos/Jun binding pairs and Ig CH1 polypeptide sequences and light chain constant region C_{L} sequences form interspecific binding pairs. Coiled-coil sequences, including leucine zipper sequences, can be either interspecific leucine zipper or non-interspecific leucine zipper sequences. See, e.g., Zeng et al., (1997) PNAS (USA) 94:3673-3678; and Li et al., (2012), Nature Comms. 3:662.

The scaffold polypeptides used to form a duplex CIIC may each comprise a leucine zipper polypeptide sequence. The leucine zipper polypeptides bind to one another to form a dimer. Non-limiting examples of leucine-zipper polypeptides include a peptide comprising any one of the following aa sequences: RMKQIEDKIEEILSKIYH IENEIARIKKLIGER (SEQ ID NO:220); LSSIEKKQEEQTS WLIWISNELTLIRNELAQS (SEQ ID NO:221); LSSIEKK LEEITSQLIQISNELTLIRNELAQ (SEQ ID NO:222); LSSIEKKLEEITSQLIQIRNELTLIRNELAQ (SEQ ID NO:223); LSSIEKKLEEITSQLQQIR NE LTLIRNELAQ (SEQ ID NO:224); LSSLEKKLEELTSQLIQLRNELTLLRNELAQ (SEQ ID NO:225); ISSLEKKIEELTSQIQQLRNEITLLRNEIAQ (SEQ ID NO:226). In some cases, a leucine zipper polypeptide comprises the following aa sequence: LEIEAAFLERENTALETRVAELRQRVQRLRNRVSQYRTRYG PLGGGK (SEQ ID NO:227). Additional leucine-zipper polypeptides are known in the art, a number of which are suitable for use as scaffold polypeptide sequences.

The scaffold polypeptide used to form a CIIC duplex may comprise a coiled-coil polypeptide sequence that forms a dimer. Non-limiting examples of coiled-coil polypeptides include, for example, a peptide of any one of the following aa sequences: LKSVENRLAWENQLKTVIEELKTVKDLLSN (SEQ ID NO:228); LARIEEKLKTIKAQLSEIA STLNMIREQLAQ (SEQ ID NO:229); VSRLEEKVKTL KSQVTELASTVSLLREQVAQ (SEQ ID NO:230); IQSEKKIED ISSLIGQIQSEITLIRNEIAQ (SEQ ID NO:231); and LMSLEKKLEELTQTLMQLQNELSMLKNELAQ (SEQ ID NO:232).

The scaffold polypeptide sequences used to form a CIIC duplex may each comprise at least one cysteine residue that can form a disulfide bond permitting homodimerization or heterodimerization of those polypeptides stabilized by an interchain disulfide bond between the cysteine residues. Examples of such aa sequences include: VDLEGSTSNGRQCAGIRL (SEQ ID NO:233); EDDVTTTEELAPALVPPPKGTCAGWMA (SEQ ID NO:234); and GHDQETTTQGPGVLLPLPKGACTGQMA (SEQ ID NO:235).

Some scaffold polypeptide sequences permit formation of CIIC complexes of higher order than duplexes, such as triplexes, tetraplexes, pentaplexes or hexaplexes. Such aa sequences include, but are not limited to, IgM constant regions (discussed below). Collagen domains, which form trimers, can also be employed. Collagen domains may comprise the three aa sequence Gly-Xaa-Xaa and/or Gly-Xaa-Yaa, where Xaa and Yaa are independently any aa, with the sequence appearing or being repeated multiple times (e.g., from 10 to 40 times). In Gly-Xaa-Yaa sequences, Xaa and Yaa are frequently proline and hydroxyproline, respectively, in greater than 25%, 50%, 75%, 80%, 90% or 95% of the Gly-Xaa-Yaa occurrences, or in each of the Gly-Xaa-Yaa occurrences. In some cases, a collagen domain comprises the sequence Gly-Xaa-Pro, which is repeated from 10 to 40 times. A collagen oligomerization peptide can comprise the following aa sequence: VTAFSNMDDM L QKAHLVIE GTFIYLRDS TEFFIRVRD GWKKLQLGE LIPIPADSP PPPALSSNP (SEQ ID NO:236).

### b) Immunoglobulin Fc Scaffold Polypeptides

Scaffold polypeptide sequences include, but are not limited to, interspecific and non-interspecific Ig Fc polypeptide sequences. However, where an Ig Fc polypeptide is employed as a scaffold polypeptide in a CIIC, the Ig Fc aa sequence may contain mutations that will prevent the spontaneous formation of CIIC duplexes (dimers) or other higher order complexes. (See, e.g., Ying et al., J. Biol. Chem., 287 (23), pp 19399-19408 (Jun. 1, 2012)).

Immunoglobulin constant regions may also include mutations (e.g., the LALA mutations discussed below) that substantially reduce or eliminate the ability of the Ig polypeptide to induce cell lysis, e.g., through complement-dependent cytotoxicity (CDC) and/or antibody-dependent cellular cytotoxicity (ADCC).

### (1) Non-Interspecific Immunoglobulin Fc Scaffold Polypeptides

The scaffold polypeptide sequences used to make higher order CIIC complexes include Ig Fc polypeptide sequences. The Ig Fc polypeptide of a CIIC can be, for example, from an IgA, IgD, IgE, IgG, or IgM, any of which may be a human polypeptide sequence, a humanized polypeptide sequence, an Ig Fc region of a synthetic heavy chain constant region, or a consensus heavy chain constant region. In embodiments, the Ig Fc polypeptide can be from a human IgG1 Fc, a human IgG2 Fc, a human IgG3 Fc, a human IgG4 Fc, a human IgA Fc, a human IgD Fc, a human IgE Fc, a human IgM Fc, etc. In some cases, the Fc polypeptide comprises an aa sequence having at least about 85% (e.g., at least about 90%, at least about 95%, at least about 98%, or at least about 99%) or 100% aa sequence identity to at least 175 contiguous aas (e.g., at least 180, at least 190, at least 200, or at least 210 contiguous aas) or all aas of an Ig Fc region depicted in FIGS. 2A-2H. In particular, the C-terminal lysine provided in some of the sequences provided in FIGS. 2A-2H (e.g., the IgG sequences in FIGS. 2D, 2E, 2F, and 2G) may be removed during cellular processing of CIIC scaffolds and may not be present on some or all of the CIICs bearing Ig scaffolds as expressed. See, e.g., van den Bremer et al. (2015) mAbs 7:4; and Sissolak et al. (2019) J. Industrial Microbiol. & Biotechnol. 46:1167. In some instances, the Fc scaffold polypeptide sequences include naturally occurring cysteine residues (or non-naturally occurring cysteine residues provided in the sequence using the tools of molecular biology to place the cysteines in the sequence, e.g., as aa substitutions or insertions) that are capable of forming interchain disulfide bonds covalently linking together two scaffold sequences and, accordingly, two CIICs. Most immunoglobulin Ig Fc scaffold polypeptides, e.g., IgG1 Fc polypeptides, and particularly those comprising only or largely wt. sequences, may spontaneously link together via disulfide bonds to form homodimers resulting in duplexes. In the case of IgM heavy chain constant regions in the presence of J-chains, higher order complexes may be formed. Unless stated otherwise, Ig Fc scaffold polypeptides present in CIICs or their higher order complexes do not comprise a membrane anchoring sequence (e.g., a transmembrane anchoring domain or a portion thereof sufficient to anchor the CIIC to a cell membrane).

In some embodiments, the scaffold polypeptide sequence(s) used to form duplex CIICs comprises an immunoglobulin heavy chain constant region (CH2-CH3) polypeptide sequence (see, e.g., FIGS. 2A-2H and SEQ ID NOs:1-13). In embodiments, the Ig Fc polypeptide will be a variant that substantially does not induce cell lysis, e.g., through activation of CDC and/or ADCC, and thus may include mutations that substantially reduce or eliminate the ability of the Ig polypeptide to induce cell lysis. A few examples of IgG1 Fc variants comprising mutations that substantially reduce or eliminate the ability of the IgG1 Fc polypeptide to induce cell lysis are provided in FIG. 2D (see SEQ ID NO:6, SEQ ID NO:7 and SEQ ID NO:8).

In some cases, the Ig Fc sequence has at least about 85%, at least about 90%, at least about 95%, at least about 98%, at least about 99%, or 100% aa sequence identity to an aa sequence of an Ig Fc region depicted in FIGS. 2A-2H. The Fc sequence may have at least about 90% to 100% aa sequence identity to an Fc region depicted in FIGS. 2A-2H. The Fc sequence may have at least about 95% to 100% aa sequence identity to an Fc region depicted in FIGS. 2A-2H. Such immunoglobulin sequences can covalently link CIIC polypeptides together by forming one or two interchain disulfide bonds.

A scaffold polypeptide sequence of a CIIC may comprise a sequence that has at least about 85% (e.g., at least about 90%, at least about 95%, at least about 98%, or at least about 99%) or 100% aa sequence identity to at least 180 contiguous aas (e.g., at least 185, at least 190, at least 200, or at least 205 contiguous aas) or all aas of the IgA Fc sequence depicted in FIG. 2A (SEQ ID NO:1). A scaffold polypeptide sequence of a CIIC may comprise a sequence that has at least about 85% (e.g., at least about 90%, at least about 95%, at least about 98%, or at least about 99%) or 100% aa sequence identity to at least 180 contiguous aas (e.g., at least 185, at least 190, at least 200, or at least 210 contiguous aas) or all aas of the IgD Fc sequence depicted in FIG. 2B (SEQ ID NO:2). A scaffold polypeptide sequence of a CIIC may comprise a sequence that has at least about 85% (e.g., at least about 90%, at least about 95%, at least about 98%, or at least about 99%) or 100% aa sequence identity to at least 180 contiguous aas (e.g., at least 185, at least 190, at least 200, or at least 210 contiguous aas) or all aas of the IgE Fc sequence depicted in FIG. 2C (SEQ ID NO:3). A scaffold polypeptide sequence of a CIIC may comprise a sequence that has at least about 85% (e.g., at least about 90%, at least about 95%, at least about 98%, or at least about 99%) or 100% aa sequence identity to at least 180 contiguous aas (e.g., at least 185, at least 190, at least 200, or at least 210 contiguous aas) or all aas of a wt. IgG Fc polypeptide sequence, such as the IgG1 Fc sequence depicted in FIG. 2D (SEQ ID NO:4).

A scaffold polypeptide sequence of a CIIC may comprise an aa sequence having at least about 90% (e.g., at least about 95%, at least about 98%, or at least about 99%) or 100% aa sequence identity to at least 185 contiguous aas (e.g., at least 190, at least 200, or at least 210, contiguous aas) or all aas of a human IgG1 Fc polypeptide (SEQ ID NO:5) depicted in FIG. 2D. A scaffold polypeptide sequence of a CIIC may comprise an aa sequence having at least about 95% (e.g., at least about 95%, at least about 98%, or at least about 99%) or 100% aa sequence identity to at least 185 contiguous aas (e.g., at least 190, at least 200, or at least 210, contiguous aas) or all aas of a human IgG2 Fc polypeptide depicted in FIG. 2E (SEQ ID NO:9). A scaffold polypeptide sequence of a CIIC may comprise an aa sequence having at least about 95% (e.g., at least about 95%, at least about 98%, or at least about 99%) or 100% aa sequence identity to at least 185 contiguous aas (e.g., at least 190, at least 200, or at least 210, contiguous aas), or all aas, of a human IgG3 Fc polypeptide depicted in FIG. 2F (SEQ ID NO:10). A scaffold polypeptide sequence of a CIIC may comprise an aa sequence having at least about 90% (e.g., at least about 95%, at least about 98%, or at least about 99%) or 100% aa sequence identity to at least 185 contiguous aas (e.g., at least 190, at least 200, or at least 210 contiguous aas, such as aas 99 to 327 or 111 to 327) or all aas of a human IgG4 Fc polypeptide depicted in FIG. 2G (SEQ ID NO:11). A scaffold polypeptide sequence of a CIIC may comprise an aa sequence having at least about 90% (e.g., at least about 95%, at least about 98%, or at least about 99%) or 100% aa sequence identity to at least 185 contiguous aas (e.g., at least 190, at least 200, or at least 210, contiguous aas), or all aas, of a human IgG4 Fc polypeptide depicted in FIG. 2G (SEQ ID NO:12).

A scaffold polypeptide sequence of a CIIC may comprise a sequence that has at least about 85% (at least about 90%, at least about 95%, at least about 98%, or at least about 99%) or 100% aa sequence identity to at least 180 (at least 190, at least 200, at least 225, or at least 250) contiguous aas or all aas of the IgM Fc (CH2, CH3, CH4) polypeptide sequence depicted in FIG. 2H (SEQ ID NO:13). The above-recited polypeptides of a CIIC comprising an immunoglobulin scaffold polypeptide sequence (e.g., depicted in FIGS. 2A-2H) can be covalently linked together by formation of one or two interchain disulfide bonds between cysteines in or adjacent to their hinge regions.

A scaffold sequence present in a CIIC may have at least about 85% (e.g., at least about 90% or at least about 95%) aa sequence identity to at least 175 contiguous aas (e.g., at least 180, at least 190, at least 200, or at least 210 contiguous aas) or all aas of a human IgG1 Fc polypeptide sequence depicted in FIG. 2D and comprise a substitution of N297 with an alanine (N297A substitution, or N77 as numbered in FIG. 2D, SEQ ID NO:7). Alternatively, the scaffold sequence might have at least 95% or 100% aa sequence identity to a human IgG1 Fc polypeptide depicted in FIG. 2D (SEQ ID NO:5) and comprises a substitution of N297 (e.g., with alanine). Substitutions at N297 lead to the removal of carbohydrate modifications and result in antibody sequences with reduced complement component 1q (“Clq”) binding compared to the wt. protein, and accordingly a reduction in CDC. In place of, or in addition to, N297 substitutions, a K322 substitution (K102 as shown in FIG. 2D), such as a K322A substitution, may be employed. K322 substitutions, such as a K322A substitution, show a substantial reduction in FcγR binding affinity and substantial reduction or removal of the ability of the scaffold to induce ADCC with the C1q binding and CDC functions substantially reduced or completely removed. Hezareh et al., (2001) J. Virol. 75:12161-168.

Amino acid L234 and other aas in the lower hinge region (e.g., aas 234 to 239, which correspond to aas 14-19 of SEQ ID NO:8, such as L235, G236, G237, P238, S239) of IgGs are involved in binding to the Fc gamma receptor (FcγR) and, accordingly, substitutions at that location reduce binding to the receptor (relative to the wt. protein) resulting in a reduction in ADCC. Hezareh et al., (2001) have demonstrated that the double substitutions (L234A, L235A or “LALA”) does not effectively bind either FcγR or Clq, and both ADCC and CDC functions were substantially diminished or completely removed. An Ig Fc scaffold polypeptide with such substitutions in the lower hinge region may comprise an aa sequence having at least about 85% or at least about 90% aa sequence identity to at least 180 contiguous aas (e.g., at least 190, at least 200, or at least 210 contiguous aas) or all aas of the wt. human IgG1 Fc polypeptide depicted in FIG. 2D, while including substitutions at L234 and/or L235 (L14 and L15, respectively) of the aa sequence depicted in FIG. 2D with an aa other than leucine. Alternatively, a scaffold aa sequence present in a CIIC may comprise an aa sequence depicted in FIG. 2D (e.g., the wt. human IgG1 sequence) with L234A and L235A (“LALA”) substitutions (see, e.g., SEQ ID NO:8), or a sequence having at least 90% or at least 95% aa sequence identity to at least 180 contiguous aas (e.g., at least 190, at least 200, or at least 210 contiguous aas) or all aas of any of those sequences.

A scaffold polypeptide sequence present in a CIIC may comprise an aa sequence depicted in FIG. 2D and having a substitution of P331 (P111 of the aa sequences depicted in FIG. 2D), or a sequence having at least 90% or at least 95% aa sequence identity to at least 180 contiguous aas (e.g., at least 190, at least 200, or at least 210 contiguous aas) or all aas of at least one of the sequences in FIG. 2D along with an aa other than proline at position 331 (e.g., a P331S substitution, SEQ ID NO:6). Alternatively, a scaffold aa sequence present in a CIIC may comprise an aa sequence depicted in FIG. 2D (e.g., the wt. human IgG1 sequence) with a P331 (e.g., P331A) substitution, or a sequence having at least 90% or at least 95% aa sequence identity to at least 180 contiguous aas (e.g., at least 190, at least 200, or at least 210 contiguous aas) or all aas of any of those sequences. In one embodiment, the substitution is a P331S substitution. In another embodiment, the substitution is a P331A substitution. Substitutions at P331, like those at N297, lead to reduced binding to Clq relative to the wt. protein, and thus a reduction in CDC. Substitutions of D270, K322, and/or P329 (corresponding to D50, K102, and P109 of SEQ ID NO:4 in FIG. 2D), for example with alanine, may be utilized individually or in any combination with or without a P331 substitution to reduce binding to Clq. The substitution(s) may comprise a P331S or a P331A substitution.

A scaffold polypeptide sequence present in a CIIC may comprise the aa sequence depicted in FIG. 2D (wt. human IgG1 Fc SEQ ID NO:4), except for substitutions at L234 and/or L235 (L14 and/or L15 as depicted in FIG. 2D) with aas other than leucine, and a substitution of P331 (P111 of that sequence as depicted) with an aa other than proline. In some cases, the scaffold polypeptide sequence present in a CIIC comprises the “Triple Variant” aa sequence (SEQ ID NO:6) depicted in FIG. 2D (human IgG1 Fc) comprising L234F, L235E, and P331S substitutions (corresponding to aa positions 14, 15, and 111 of the aa sequence depicted in FIG. 2D) or a sequence having all three variants and having at least 90% or at least 95% aa sequence identity to at least 180 contiguous aas (e.g., at least 190, at least 200, or at least 210 contiguous aas) or all aas of SEQ ID NO:6.

A scaffold polypeptide sequence of a CIIC may comprise an aa sequence having at least about 85% (e.g., at least about 90%, at least about 95%, at least about 98%, or at least about 99%) aa sequence identity to at least 180 contiguous aas (e.g., at least 190, at least 200, or at least 210 contiguous aas) or all aas of the wt. human IgG1 Fc polypeptide depicted in FIG. 2D, and include substitutions of D270, K322, and/or P329 (corresponding to D50, K102, and P109 of SEQ ID NO:4 in FIG. 2D) that reduce binding to Clq protein relative to the wt. proteins.

A scaffold polypeptide sequence of a CIIC may comprise an aa sequence having at least about 90%, at least about 95%, at least about 98%, or at least about 99%) or 100% aa sequence identity to at least 180 contiguous aas (e.g., at least 200, at least 250, or at least 300 contiguous aas) of a human IgM heavy chain such as that set forth in SEQ ID NO:13 (see, e.g., FIG. 2H), which forms hexamers, or pentamers (particularly when combined with a mature j-chain peptide lacking a signal sequence such as that provided in FIG. 21).

### (2) Interspecific Immunoglobulin Fc Scaffold Polypeptides

Where an asymmetric pairing between two CIIC molecules is desired (e.g., to produce CIIC duplexes with different MODs), a scaffold polypeptide present in a CIIC may comprise, consist essentially of, or consist of interspecific Ig Fc polypeptide sequence variants. Such interspecific polypeptide sequences include, but are not limited to, knob-in-hole without (KiH) or with (KiHs-s) a stabilizing disulfide bond, HA-TF, ZW-1, 7.8.60, DD-KK, EW-RVT, EW-RVTs-s, and Δ107 sequences. One interspecific binding pair comprises a T366Y and Y407T mutant pair in the CH3 domain interface of IgG1, or the corresponding residues of other immunoglobulins. See Ridgway et al., Protein Engineering 9:7, 617-621 (1996). A second interspecific binding pair involves the formation of a knob by a T366W substitution, and a hole by the triple substitutions T366S, L368A and Y407V on the complementary Ig Fc sequence. See Xu et al. mAbs 7:1, 231-242 (2015). Another interspecific binding pair has a first Ig Fc polypeptide with Y349C, T366S, L368A, and Y407V substitutions and a second Ig Fc polypeptide with S354C and T366W substitutions (disulfide bonds can form between the Y349C and the S354C). See, e.g., Brinkmann et al., mAbs 9:2, 182-212 (2015). Ig Fc polypeptide sequences, either with or without knob-in-hole modifications, can be stabilized by the formation of disulfide bonds between the Ig Fc polypeptides (e.g., the hinge region disulfide bonds). Several interspecific binding sequences based upon immunoglobulin sequences are summarized in Table 3 that follows, with cross reference to the numbering of the aa positions as they appear in the wt. IgG1 sequence (SEQ ID NO:4) set forth in FIG. 2D shown in brackets “{ }”.

TABLE 3
Pairs of interspecific immunoglobulin Fc sequences and
their cognate counterpart interspecific sequences

| | Substitutions in the first | Substitutions in the second | |
|---|---|---|---|
| Interspecific | interspecific polypeptide | (counterpart) interspecific | |
| Pair Name | sequence | polypeptide sequence | Comments |
| KiH | T366W | T366S/L368A/Y407V | Hydrophobic/steric |
| | {T146W} | {T146S/L148A/Y187V} | complementarity |
| KiHs-s | T366W/S354C* | T366S/L368A/Y407V/Y349C | KiH + inter-CH3 |
| | {T146W/S134C*} | {T146S/L148A/Y187V/Y129C} | domain S—S bond |
| HA-TF | S364H/F405A | Y349T/T394F | Hydrophobic/steric |
| | {S144H/F185A} | {Y129T/T174F} | complementarity |
| ZW1 | T350V/L351Y/F405A/Y407V | T350V/T366L/K392L/T394W | Hydrophobic/steric |
| | {T130V/L131Y/F185A/Y187V} | {T130V/T146L/K172L/T174W} | complementarity |
| 7.8.60 | K360D/D399M/Y407A | E345R/Q347R/T366V/K409V | Hydrophobic/steric |
| | {K140D/D179M/Y187A} | {E125R/Q127R/T146V/K189V} | complementarity + |
| | | | electrostatic |
| | | | complementarity |
| DD-KK | K409D/K392D | D399K/E356K | Electrostatic |
| | {K189D/K172D} | {D179K/E136K} | complementarity |
| EW-RVT | K360E/K409W | Q347R/D399V/F405T | Hydrophobic/steric |
| | {K140E/K189W} | {Q127R/D179V/F185T} | complementarity & |
| | | | long-range electro- |
| | | | static interaction |
| EW-RVTs-s | K360E/K409W/Y349C* | Q347R/D399V/F405T/S354C | EW-RVT + inter-CH3 |
| | {K140E/K189W/Y129C*} | {Q127R/D179V/F185T/S134C} | domain S—S bond |
| A107 | K370E/K409W | E357N/D399V/F405T | Hydrophobic/steric |
| | {K150E/K189W} | {E137N/D179V/F185T} | complementarity + |
| | | | hydrogen bonding |
| | | | complementarity |

Table 3 is modified from Ha et al., Frontiers in Immunol. 7: 1-16 (2016).
*aa forms a stabilizing disulfide bond.

In addition to the interspecific pairs of sequences in Table 3, scaffold polypeptides may include interspecific “SEED” sequences having 45 residues derived from IgA in an IgG1 CH3 domain of the interspecific sequence and 57 residues derived from IgG1 in the IgA CH3 in its counterpart interspecific sequence. See Ha et al., Frontiers in Immunol.7:1-16 (2016).

Interspecific immunoglobulin sequences may include substitutions described above for non-interspecific immunoglobulin sequences that inhibit binding either or both of the FcγR or Clq, and reduce or abolish ADCC and CDC function.

In an embodiment, a scaffold polypeptide found in a CIIC may comprise an interspecific binding sequence or its counterpart interspecific binding sequence selected from the group consisting of: knob-in-hole (KiH); knob-in-hole with a stabilizing disulfide (KiHs-s); HA-TF; ZW-1; 7.8.60; DD-KK; EW-RVT; EW-RVTs-s; Δ107; or SEED sequences.

In an embodiment, a CIIC comprises a scaffold polypeptide comprising an IgG1 sequence with a T146W KiH sequence substitution, and its counterpart interspecific binding partner polypeptide comprises an IgG1 sequence having T146W, L148A, and Y187V KiH sequence substitutions, where the scaffold polypeptides comprise a sequence having at least 85%, at least 90%, at least 95%, or at least 97% aa sequence identity to at least 100 (e.g., at least 125, at least 150, at least 170, at least 180, at least 190, at least 200, at least 210, at least 220, or all 227) contiguous aas of the wt. IgG1 of FIG. 2D. Scaffold polypeptides optionally comprise substitutions at one of more of: L234 and L235 (e.g., L234A/L235A “LALA” or L234F/L235E); N297 (e.g., N297A); P331 (e.g., P331 S); L351 (e.g., L351K); T366 (e.g., T366S); P395 (e.g., P395V); F405 (e.g., F405R); Y407 (e.g., Y407A); and K409 (e.g., K409Y). Those substitutions appear at: L14 and L15 (e.g., L14A/L15A “LALA” or L14F/L15E); N77 (e.g., N77A); P111 (e.g., P111S); L131 (e.g., L131K); T146 (e.g., T146S); P175 (e.g., P175V); F185 (e.g., F185R); Y187 (e.g., Y187A); and K189 (e.g., K189Y) in the wt. IgG1 sequence of FIG. 2D.

In an embodiment, a CIIC or duplex CIIC comprises a scaffold polypeptide comprising an IgG1 sequence with a T146W KiH sequence substitution, and its counterpart interspecific binding partner polypeptide comprises an IgG1 sequence having T146S, L148A, and Y187V KiH sequence substitutions, where the scaffold polypeptides comprise a sequence having at least 85%, at least 90%, at least 95%, or at least 97% aa sequence identity to at least 100 (e.g., at least 125, at least 150, at least 170, 1 at least 80, at least 190, at least 200, at least 210, at least 220, or all 227) contiguous aas of the wt. IgG1 of FIG. 2D, where one or both (in the case of a duplex CIIC) scaffold polypeptide sequence(s) may comprise additional substitutions such as L14 and/or L15 substitutions (e.g., “LALA” substitutions L234A and L235A) and/or N77 (N297 e.g., N297A or N297G).

In an embodiment, a CIIC or duplex CIIC comprises a scaffold polypeptide comprising an IgG1 sequence with T146W and S134C KiHs-s substitutions, and its counterpart interspecific binding partner polypeptide comprises an IgG1 sequence having T146S, L148A, Y187V and Y129C KiHs-s substitutions, where the scaffold polypeptides comprise a sequence having at least 85%, at least 90%, at least 95%, or at least 97% aa sequence identity to at least 100 (e.g., at least 125, at least 150, at least 170, at least 180, at least 190, at least 200, at least 210, at least 220, or all 227) contiguous aas of the wt. IgG1 of FIG. 2D, where one or both (in the case of a duplex CIIC) scaffold polypeptide sequence(s) may comprise additional substitutions such as L14 and/or L15 substitutions (e.g., “LALA” substitutions L234A and L235A) and/or an N77 substitution (N297 e.g., N297A or N297G).

In an embodiment, a CIIC comprises a scaffold polypeptide comprising an IgG1 sequence with S144H and F185A HA-TF substitutions, and its counterpart interspecific binding partner polypeptide comprises an IgG1 sequence having Y129T and T174F HA-TF substitutions, where the scaffold polypeptides comprise a sequence having at least 85%, at least 90%, at least 95%, or at least 97% aa sequence identity to at least 100 (e.g., at least 125, at least 150, at least 170, at least 180, at least 190, at least 200, at least 210, at least 220, or all 227) contiguous aas of the wt. IgG1 of FIG. 2D, where one or both (in the case of a duplex CIIC) scaffold polypeptide sequence(s) may comprise additional substitutions such as L14 and/or L15 substitutions (e.g., “LALA” substitutions L234A and L235A) and/or an N77 substitution (N297 e.g., N297A or N297G).

In an embodiment, a CIIC or duplex CIIC comprises a scaffold polypeptide comprising an IgG1 sequence with T130V, L131Y, F185A, and Y187V ZW1 substitutions, and its counterpart interspecific binding partner polypeptide comprises an IgG1 sequence havingT130V, T146L, K172L, and T174W ZW1 substitutions, where the scaffold polypeptides comprise a sequence having at least 85%, at least 90%, at least 95%, or at least 97% aa sequence identity to at least 100 (e.g., at least 125, at least 150, at least 170, at least 180, at least 190, at least 200, at least 210, at least 220, or all 227) contiguous aas of the wt. IgG1 of FIG. 2D, where one or both (in the case of a duplex CIIC) scaffold polypeptide sequence(s) may comprise additional substitutions such as L14 and/or L15 substitutions (e.g., “LALA” substitutions L234A and L235A) and/or an N77 substitution (N297 e.g., N297A or N297G).

In an embodiment, a CIIC or duplex CIIC comprises a scaffold polypeptide comprising an IgG1 sequence with K140D, D179M, and Y187A 7.8.60 substitutions, and its counterpart interspecific binding partner polypeptide comprises an IgG1 sequence having T130V, E125R, Q127R, T146V, and K189V 7.8.60 substitutions, where the scaffold polypeptides comprise a sequence having at least 85%, at least 90%, at least 95%, or at least 97% aa sequence identity to at least 100 (e.g., at least 125, at least 150, at least 170, at least 180, at least 190, at least 200, at least 210, at least 220, or all 227) contiguous aas of the wt. IgG1 of FIG. 2D, where one or both (in the case of a duplex CIIC) scaffold polypeptide sequence(s) may comprise additional substitutions such as L14 and/or L15 substitutions (e.g., “LALA” substitutions L234A and L235A) and/or an N77 substitution (N297 e.g., N297A or N297G).

In an embodiment, a CIIC or duplex CIIC comprises a scaffold polypeptide comprising an IgG1 sequence with K189D and K172D DD-KK substitutions, and its counterpart interspecific binding partner polypeptide comprises an IgG1 sequence having T130V, D179K, and E136K DD-KK substitutions, where the scaffold polypeptides comprise a sequence having at least 85%, at least 90%, at least 95%, or at least 97% aa sequence identity to at least 100 (e.g., at least 125, at least 150, at least 170, at least 180, at least 190, at least 200, at least 210, at least 220, or all 227) contiguous aas of the wt. IgG1 of FIG. 2D, where one or both (in the case of a duplex CIIC) scaffold polypeptide sequence(s) may comprise additional substitutions such as L14 and/or L15 substitutions (e.g., “LALA” substitutions L234A and L235A) and/or an N77 substitution (N297 e.g., N297A or N297G).

In an embodiment, a CIIC or duplex CIIC comprises a scaffold polypeptide comprising an IgG1 sequence with K140E and K189W EW-RVT substitutions, and its counterpart interspecific binding partner polypeptide comprises an IgG1 sequence having T130V, Q127R, D179V, and F185T EW-RVT substitutions, where the scaffold polypeptides comprise a sequence having at least 85%, at least 90%, at least 95%, or at least 97% aa sequence identity to at least 100 (e.g., at least 125, at least 150, at least 170, at least 180, at least 190, at least 200, at least 210, at least 220, or all 227) contiguous aas of the wt. IgG1 of FIG. 2D, where one or both (in the case of a duplex CIIC) scaffold polypeptide sequence(s) may comprise additional substitutions such as L14 and/or L15 substitutions (e.g., “LALA” substitutions L234A and L235A) and/or an N77 substitution (N297 e.g., N297A or N297G).

In an embodiment, a CIIC or duplex CIIC comprises a scaffold polypeptide comprising an IgG1 sequence with K140E, K189W, and Y129C EW-RVTs-s substitutions, and its counterpart interspecific binding partner polypeptide comprises an IgG1 sequence having T130V, Q127R, D179V, F185T, and S134C EW-RVTs-s substitutions, where the scaffold polypeptides comprise a sequence having at least 85%, at least 90%, at least 95%, or at least 97% aa sequence identity to at least 100 (e.g., at least 125, at least 150, at least 170, at least 180, at least 190, at least 200, at least 210, at least 220, or all 227) contiguous aas of the wt. IgG1 of FIG. 2D, where one or both (in the case of a duplex CIIC) scaffold polypeptide sequence(s) may comprise additional substitutions such as L14 and/or L15 substitutions (e.g., “LALA” substitutions L234A and L235A) and/or an N77 substitution (N297 e.g., N297A or N297G).

In an embodiment, a CIIC or duplex CIIC comprises a scaffold polypeptide comprising an IgG1 sequence with K150E and K189W Δ107 substitutions, and its counterpart interspecific binding partner polypeptide comprises an IgG1 sequence having T130V, E137N, D179V, and F185T Δ107 substitutions, where the scaffold polypeptides comprise a sequence having at least 85%, at least 90%, at least 95%, or at least 97% aa sequence identity to at least 100 (e.g., at least 125, at least 150, at least 170, at least 180, at least 190, at least 200, at least 210, at least 220, or all 227) contiguous aas of the wt. IgG1 of FIG. 2D, where one or both (in the case of a duplex CIIC) scaffold polypeptide sequence(s) may comprise additional substitutions such as L14 and/or L15 substitutions (e.g., “LALA” substitutions L234A and L235A) and/or an N77 substitution (N297 e.g., N297A or N297G).

As an alternative to the use of immunoglobulin CH2 and CH3 heavy chain constant regions as scaffold sequences, immunoglobulin light chain constant regions (see FIG. 3) can be paired with Ig CH1 sequences (see, e.g., FIG. 21) as interspecific scaffold sequences.

In an embodiment, a CIIC scaffold polypeptide comprises an Ig CH1 domain (e.g., the polypeptide of FIG. 21, SEQ ID NO:14), and the sequence with which it will form a complex (its counterpart binding partner) comprises an Ig K chain constant region sequence, where the scaffold polypeptide comprises a sequence having at least 85%, at least 90%, at least 95%, at least 98%, at least 99%, or 100% aa sequence identity to at least 70, at least 80, at least 90, at least 100, or at least 110 contiguous aas of SEQ ID NO:16. (See FIGS. 21 and 3.) The Ig CH1 and Ig K sequences may be modified to increase their affinity for each other and, accordingly, the stability of any heterodimer formed utilizing them. Among the substitutions that increase the stability of CH1-Ig K heterodimers are those identified as the MD13 combination in Chen et al., MAbs, 8(4):761-774 (2016). In the MD13 combination, two substitutions are introduced into each of the IgCH1 and Ig K sequences. The Ig CH1 sequence is modified to contain S64E and S66V substitutions (S70 and S72 of the sequence shown in FIG. 21). The Ig K sequence is modified to contain S69L and T71S substitutions (S68 and T70 of the sequence shown in FIG. 3).

In another embodiment, a scaffold polypeptide of a CIIC comprises an Ig CH1 domain (e.g., the polypeptide of FIG. 2I (SEQ ID NO:14), and its counterpart sequence comprises an Ig A chain constant region sequence such as is shown in FIG. 3B (SEQ ID NO:17), where the scaffold polypeptide comprises a sequence having at least 85%, at least 90%, at least 95%, at least 98%, at least 99%, or 100% aa sequence identity to at least 70 (e.g., at least 80, at least 90, or at least 100) contiguous aas of the sequence shown in FIG. 21.

### 4. Membrane Association Sequences

CIICs may comprise a MAS that comprises amino acid residues that directly interact with the hydrophobic lipid portion of a lipid bilayer resulting in the CIIC becoming anchored into the membrane as an integral membrane protein. A MAS may take the form of a single transmembrane domain sequence, multiple transmembrane domain sequences that cross a cell membrane multiple times, or an amphipathic a helix that partitions into a monolayer of a lipid bilayer (a monotopic membrane interaction). For the purpose of this disclosure, post-translational modification sequences that result in the formation of integral membrane proteins due to the addition of hydrophobic groups (e.g., lipids or prenyl groups) are treated as additional polypeptide sequences. The strength of the interaction between an amphipathic helix and a lipid bilayer determines how tightly associated a protein containing the helix is associated with the membrane with weak interactions leading to amphitropic behavior (see, e.g., Johnson et al., Mol. Mem. Biol. 12:217-235 (1999)). A MAS may appear in a CIIC either in place of a scaffold sequence or in addition to a scaffold sequence. A MAS, particularly in the form of a transmembrane domain or amphipathic helix, when present in a CIIC is generally located at or near the C-terminus of the CIIC (e.g., on the C-terminal side of the α2 domain and any scaffold sequence that may be present in addition to the MAS, see FIG. 1). Locating the MAS at or near the C-terminus avoids having other sequences improperly displayed on the intracellular side of the membrane. As discussed below, CIICs may comprise the transmembrane domain sequence of an MHC α or β subunit.

In structures J-M of FIG. 1 some embodiments of CIICs associated with a lipid bilayer membrane (1) via a transmembrane aa sequence are illustrated. In structures J and K, the MAS is linked to the α2 domain by an optional L3 linker and/or a membrane proximal sequence. In K the transmembrane domains interact to form a duplex of CIICs that are integral membrane proteins. In structures L and M, the CIICs each comprise from N-terminus to C-terminus an optional L3 linker, a scaffold sequence, an optional L4 linker, and a transmembrane MAS. In structure M, the CIICs form a duplex of CIICs that are integral membrane proteins associated by their scaffold sequences. The dashed lines in structures J to M represent potential body disulfide bonds that may be present in any of the structures shown; however, linker disulfide bonds may replace the body disulfide bonds.

Transmembrane domains of MHC proteins may be utilized as MAS in CIICs. Where there is no scaffold sequence present in the CIIC the transmembrane sequence may be linked directly to the α2 domain sequence. Alternatively, the transmembrane domain may be attached to the α2 domain via an MHC membrane proximal region or a membrane proximal region and L3 linker. Exemplary structures of the carboxyl terminal portion of CIICs starting with the α2 domain include: (I)-α2 domain-transmembrane domain; (Σ)-α2 domain-membrane proximal region-transmembrane domain; (Σ)-α2 domain-membrane proximal region-L3-transmembrane domain; or (Σ)-α2 domain-membrane proximal region-L3-transmembrane domain; where the “(Σ)-” stands for the epitope through the α1 domain including any Lα linker that may be present (see, e.g., FIG. 1).

Where a scaffold sequence is present in a CIIC, the transmembrane sequence may be linked directly to the scaffold sequence. Alternatively, the transmembrane domain may be attached to the scaffold sequence via an L4 linker. Exemplary structures of the carboxyl terminal portion of CIICs having a scaffold sequence and transmembrane MAS starting with the α2 domain include: (Σ)-α2 domain-scaffold-transmembrane domain; (Σ)-α2 domain-membrane proximal region-scaffold-transmembrane domain; (Σ)-α2 domain-membrane proximal region-L3-scaffold-transmembrane domain; or (Σ)-α2 domain-membrane proximal region-L3-scaffold-L4-transmembrane domain; where the “(Σ)-” stands for the epitope through the α1 domain including any Lα linker that may be present (see, e.g., FIG. 1).

Where an MHC transmembrane domain sequence is used as a MAS of a CIIC, the MAS may be taken from the sequence of the α or β subunit present in the CIIC. In particular, the MHC transmembrane domain, and the membrane proximal domain if present, may be taken from the same allele as the α2 domain. In such a case, the structure of the carboxyl terminal portion of the CIIC starting with the α2 domain may be that of a naturally occurring allele. For example, where the CIIC comprises the α2 domain of HLA DRA*01:02, the sequence starting with the α2 domain may comprise aas 85 to 214 of SEQ ID NO:18 provided in FIG. 4 (aas 85-229 where the intracellular domain is also included) or a sequence having at least 90% or at least 95% aa sequence identity with that region of the protein. Similarly, where CIICs comprise DP alleles the CIIC may comprise the sequence from aas 88 to 216 (or 232 if the intracellular domain is included, see FIG. 9) of a DP a allele, or a sequence having at least 90% or at least 95% aa sequence identity with that region of a DP a allele. For CIICs comprising DQ alleles, the CIIC may comprise the sequence from aa 86/87 to aa 216/217 depending on the specific DQA1 or DQA2 allele (aa 216/217 to aa 231/232 if the intracellular domain is included, see FIGS. 11 and 12), or a sequence having at least 90% or at least 95% aa sequence identity with that region of a DQ a allele.

In addition to MHC transmembrane domains, where a CIIC comprises a MAS it may be derived from non-MHC proteins. For example, the transmembrane domain of glycophorin A (GPA) protein, which can dimerize, may be used as a transmembrane domain (see, e.g., NCBI Ref. Seq. NP_002090.4 and Lemmon et al. J. Biol. Chem., 267 (11), 7683-7689 (1992)). Similarly, the transmembrane domain of small integral membrane protein 1 (SMIM1) may be employed as a transmembrane domain (see, e.g., NCBI Ref. Seq. NP_001157196.1). Amphipathic helices, such as that of cytidylyltransferase, ADP Ribosylation Factor, blood-clotting factor VIII, vinculin, and DnaA (see, e.g., Johnson and Cornell, Mol. Mem. Biol. 12:217-235 (1999)) may also be used as a MAS in CIICs.

For the purpose of this disclosure, otherwise soluble CIICs may be converted into membrane proteins by the addition of sequences resulting in post-translational modifications that lead to association of the CIIC with lipid bilayers. These are discussed under Additional Polypeptide Sequences as a form of post-translational modification sequence. For example, aa sequences that result in direct or indirect covalent attachment to a lipid or prenyl group or glycosylphosphatidylinositol may be added to CIICs. In an embodiment, a farnesyltransferase or geranylgeranyl transferase motif may be located at the COOH-terminus of proteins.

### 5. Linkers

As noted above, a CIIC can include a linker sequence (aa, peptide, or polypeptide linker sequence) or “linker” interposed between any two elements of a CIIC, e.g., between an epitope and an MHC polypeptide, between an MHC polypeptide and an Ig Fc polypeptide, between a first MHC polypeptide and a second MHC polypeptide, etc.

Although termed “linkers,” sequences employed for linkers may also be placed at the N- and/or C-terminus of a CIIC polypeptide to, for example, stabilize the CIIC polypeptide (e.g., increase its thermal stability and/or prevent nonspecific aggregation) or protect it from proteolytic degradation. Linkers are understood not to comprise, consist essentially of, or consist of, functional elements such as portions of MHC sequences, and any such elements may be excluded from any linker present in a CIIC by proviso.

Suitable polypeptides for use as L1, L2, L3, L4, Lα, Lβ or other linkers (also referred to as “spacers”) in a CIIC are known in the art, can be readily selected and can be of any of a number of suitable lengths, e.g., from 2 to about 50 aas in length, e.g., from about 2 aas to about 10 aas, from about 10 aas to about 20 aas, from about 20 aas to about 30 aas, from about 30 aas to about 40 aas, from about 40 aas to about 50 aas, or longer than about 50 aas. In embodiments, a suitable linker can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 aas in length. L1 linkers are typically from about 8 to about 16 aas including e.g., 12 aas in length. L2 linkers are typically from about 16 to about 24 aas including e.g., about 20 aas in length. L3 linkers are typically from about 6 to about 14 aas including e.g., 10 aas in length. While Lα and/or Lp linkers may be present in CIICs, they typically are not present. Linkers can be generally classified into three groups, i.e., flexible, rigid, and cleavable. See, e.g., Chen et al. (2013) Adv. Drug Deliv. Rev. 65:1357, and Klein et al. (2014) Protein Engineering, Design & Selection 27:325. Unless stated otherwise, the peptide linkers in a CIIC of this disclosure, and particularly the L1, L2, L3, L4, Lα, and Lβ linkers, are not cleavable by sequence specific proteases generally known in the art (e.g., linkers are not cleavable by site specific proteases giving rise to a single cleavage in a CIIC linker), although as polypeptides they may be subject to endopeptidase and/or exopeptidase action.

Polypeptide linkers in the CIIC may include, for example, polypeptides that comprise, consist essentially of, or consist of: i) Gly and Ser, ii) Ala and Ser, iii) Gly, Ala, and Ser, iv) Gly, Ser, and Cys (e.g., a single Cys residue), v) Ala, Ser, and Cys (e.g., a single Cys residue), and vi) Gly, Ala, Ser, and Cys (e.g., a single Cys residue). Exemplary linkers may comprise glycine polymers, glycine-serine polymers, glycine-alanine polymers, and alanine-serine polymers, including, for example polymers comprising the sequences GGSS (SEQ ID NO:239) which may be repeated or appear from 1 to 10 times (SEQ ID NOs:239 and 383 to 391), GSGGS (SEQ ID NO:240) which may be repeated or appear from 1 to 10 times (SEQ ID NOs:240 and 392 to 400), or GGGS ((Gly)₃Ser or G₃S, SEQ ID NO:238), which may be repeated or appear from 1 to 10 times (SEQ ID NOs:238 and 401 to 409), and other flexible linkers known in the art. Glycine and glycine-serine polymers can both be used, both Gly and Ser are relatively unstructured and therefore can serve as a neutral tether between components. Glycine polymers access significantly more phi-psi space than even alanine, and are much less restricted than residues with longer side chains (see Scheraga, Rev. Computational Chem. 11173-142 (1992)). Exemplary linkers may also comprise an aa sequence comprising, but not limited to, GGSG (SEQ ID NO:241) which may be repeated or appear from 1 to 10 times (SEQ ID NOs:241 and 410 to 418), GGSGG (SEQ ID NO:242) which may be repeated or appear from 1 to 10 times (SEQ ID NOs:242 and 419 to 427), GSGSG (SEQ ID NO:243) which may be repeated or appear from 1 to 10 times (SEQ ID NOs:243 and 428 to 436), GSGGG (SEQ ID NO:244) which may be repeated or appear from 1 to 10 times (SEQ ID NOs:244 and 437 to 445), GGGSG (SEQ ID NO:245) which may be repeated or appear from 1 to 10 times (SEQ ID NOs:245 and 446 to 454), GSSSG (SEQ ID NO:246) which may be repeated or appear from 1 to 10 times (SEQ ID NOs:246 and 455 to 463), or combinations thereof, and the like. Linkers can also comprise the sequence Gly(Ser)₄ (SEQ ID NO:247) which may be repeated or appear from 1 to 10 times (SEQ ID NOs:247 and 464 to 472) or (Gly)₄Ser or G₄S (SEQ ID NO:237) which may be repeated or appear from 1 to 10 times (SEQ ID NOs:237 and 473 to 480). A linker may comprise the aa sequence AAAGG (SEQ ID NO:248) which may be repeated or appear from 1 to 10 times (SEQ ID NOs:248 and 481 to 489) or the aa sequence GGSAAAGG (SEQ ID NO:249) which may be repeated or appear from 1 to 10 times (SEQ ID NOs:249 and 491 to 498).

Rigid polypeptide linkers comprise a sequence of amino acids that effectively separates protein domains by maintaining a substantially fixed distance/spatial separation between the domains, thereby reducing or substantially eliminating unfavorable interactions between such domains. Rigid polypeptide linkers thus may be employed where it is desired to minimize the interaction between the domains of the CIIC. For example, where MODs (e.g., IL-2) are located on the carboxy terminus of a CIIC, e.g., C-terminal to a scaffold sequence (e.g., an Ig Fc sequence), a rigid linker may be employed between the scaffold sequence and the MOD. Rigid peptide linkers include peptide linkers rich in proline, and peptide linkers having an inflexible helical structure, such as an α-helical structure. Examples of rigid peptide linkers include, e.g., (EAAAK) (SEQ ID NO:250), A(EAAAK)A (SEQ ID NO:251), A(EAAAK)ALEA(EAAAK)A (SEQ ID NO:252), (Lys-Pro), (Glu-Pro), (Thr-Pro-Arg), and (Ala-Pro) where the bracketed sequences may be repeated or appear from 1 to 20 times (e.g., 1, 2, 3, 4, 5, 6, 7, 8, 9, 10, 11, 12, 13, 14, 15, 16, 17, 18, 19, or 20). Non-limiting examples of suitable rigid linkers comprising EAAAK (SEQ ID NO:250) include EAAAK (SEQ ID NO:250), (EAAAK)₂ (SEQ ID NO:253), (EAAAK)₃ (SEQ ID NO:254), A(EAAAK)ALEA(EAAAK)A (SEQ ID NO:252), where the EAAAK sequence may be repeated or appear 1-4 times, and AEAAAKEAAAKA (SEQ ID NO:255). Non-limiting examples of suitable rigid linkers comprising (AP)n include APAP (SEQ ID NO:256, also referred to herein as “(AP)2”), APAPAPAP (SEQ ID NO:257), also referred to herein as “(AP)4”), APAPAPAPAPAP (SEQ ID NO:258), also referred to herein as “(AP)6”), APAPAPAPAPAPAPAP (SEQ ID NO:259), also referred to herein as “(AP)8”), and APAPAPAPAPAPAPAPAPAP (SEQ ID NO:260), also referred to herein as “(AP)10”). Non-limiting examples of suitable rigid linkers comprising (KP)n include KPKP (SEQ ID NO:261), also referred to herein as “(KP)2”), KPKPKPKP (SEQ ID NO:262), also referred to herein as “(KP)4”), KPKPKPKPKPKP (SEQ ID NO:263), also referred to herein as “(KP)6”), KPKPKPKPKPKPKPKP (SEQ ID NO:264), also referred to herein as “(KP)8”), and KPKPKPKPKPKPKPKPKPKP (SEQ ID NO:265), also referred to herein as “(KP)10”). Non-limiting examples of suitable rigid linkers comprising (EP)n include EPEP (SEQ ID NO:266), also referred to herein as “(EP)2”), EPEPEPEP (SEQ ID NO:267), also referred to herein as “(EP)4”), EPEPEPEPEPEP (SEQ ID NO:268), also referred to herein as “(EP)6”), EPEPEPEPEPEPEPEP (SEQ ID NO:269), also referred to herein as “(EP)8”), and EPEPEPEPEPEPEPEPEPEP (SEQ ID NO:270), also referred to herein as “(EP)10”).

A linker polypeptide present in a polypeptide of a CIIC may include a cysteine residue that can form a disulfide bond with a cysteine residue present in another polypeptide of the CIIC. In particular, an L1 linker between the epitope and the β1 domain sequence of a CIIC may contain a cysteine that can form a linker disulfide bond with a cysteine in the α1 domain of the MHC α subunit sequence of the CIIC. In some cases, for example, the linker comprises an aa sequence selected from CGGGS (SEQ ID NO:271), GCGGS (SEQ ID NO:272), GGCGS (SEQ ID NO:273), GGGCS (SEQ ID NO:274), and GGGGC (SEQ ID NO:275) with the rest of the linker comprised of Gly and Ser residues (e.g., GGGGS units (SEQ ID NO:237) that may be repeated or appear from 1 to 10 times, such as 1, 2, 3, 4, 5, 6, 7, 8, 9, or 10 times.

Accordingly, the L1 linker may be from about 5 to about 50 aas in length, and may, for example, be from about 5 to about 50 aas in length and comprise more than 50% Gly and Ser residues with one cysteine residue. The L1 may be from about 5 to about 50 aas in length, and comprise more than 50% (Gly)₄S repeats with one optional cysteine residue that may be used to form a CIIC stabilizing linker disulfide. The L1 linker may be a (Gly)₄S sequence repeated from 3 to 8 (e.g., 3 to 7) times, optionally having one aa replaced by a cysteine residue. Some cysteine containing linkers may also be selected from the sequences GCGASGGGGSGGGGS (SEQ ID NO:276), GCGGSGGGGSGGGGSGGGGS (SEQ ID NO:277), and GCGGSGGGGSGGGGS (SEQ ID NO:278). Such linkers may be considered to be substantial repeats of a (Gly)₄Ser motif (SEQ ID NO:237) with a cysteine substitution useful as an L1 linker capable of forming a CIIC stabilizing linker disulfide. Where the cysteine is at the second position (from the N-terminal point where the epitope is attached) of such linkers (from the N-terminal point where the epitope is attached) it is referred to as a 2C substitution, and when replacing a glycine at position 2 of the linker as a G2C substitution.

### 6. Epitopes

A variety of peptide epitopes (“epitope-presenting peptides”) may be present in a CIIC or higher order complexes of CIICs (such as duplex CIICs). An antigen or epitope “associated with” a particular disease or disorder, other than an autoimmune disease or disorder, is a non-self-antigen or non-self epitope that is a target of antibodies and/or reactive T cells in individuals exposed to the antigen of the disease-causing agent (e.g., a protein, bacteria, virus, or other causative agent). An antigen or epitope “associated with” a particular autoimmune disorder (including but not limited to T1D or celiac disease) is a self-antigen or epitope that is a target of autoantibodies and/or autoreactive T cells present in individuals with that autoimmune disorder, where such autoantibodies and/or autoreactive T cells mediate a pathological state associated with the autoimmune disorder. Antigens or epitopes associated with diseases or autoimmune diseases and disorders are discussed in more detail below. Peptide epitopes present in CIICs (e.g., T1D or celiac peptide epitopes) are bound to the MHC portion of the CIIC such that the peptide-MHC complex (“pMHC”) may be recognized by a TCR on the surface of a T cell specific for the pMHC. A pMHC of a CIIC (e.g., a duplex CIIC) is designed to be specifically bound by a target T cell that has a T cell receptor (“TCR”) that is specific for the epitope presented by the pMHC and that specifically binds the pMHC of the CIIC. An epitope-specific T cell thus binds a pMHC comprising a peptide epitope having a reference aa sequence, but substantially does not bind pMHC comprising a peptide that differs from the reference aa sequence. For example, an epitope-specific T cell binds a pMHC comprising a peptide epitope having a reference aa sequence, and binds a pMHC comprising a peptide that differs from the reference aa sequence, if at all, with an affinity that is less than 10⁻⁶ M, less than 10⁻⁵ M, or less than 10⁻⁴ M. An epitope-specific T cell may, for example, bind a pMHC comprising a peptide epitope for which it is specific with an affinity of at least 10⁻⁷ M, at least 10⁻⁸ M, at least 10⁻⁹ M, or at least 10¹⁰ M.

A peptide epitope (e.g., a T1D- or celiac-epitope) may have a length of from about 4 aas to about 25 aas. For example, a peptide epitope may have a length of from 4 aas to about 10 aas, or from about 10 aas to about 15 aas. Other ranges of peptide epitope length include from about 15 aas to about 20 aas, or from about 20 aas to about 25 aas. A peptide epitope present in a CIIC can have a length of 4 aas, 5 aas, 6 aas, 7 aas, 8 aas, 9 aas, 10 aas, 11 aas, 12 aas, 13 aas, 14 aas, 15 aas, 16 aas, 17 aas, 18 aas, 19 aas, 20 aas, 21 aas, 22 aas, 23 aas, 24 aas, or 25 aas. Peptide epitopes (e.g., T1D- or celiac-epitopes) present in a CIIC may have a length of from about 5 aas to about 10 aas, including 5 aas, 6 aas, 7 aas, 8 aas, 9 aas, or 10 aas.

Suitable peptide epitopes in CIICs include T1D-associated peptide epitopes and celiac-associated peptide epitopes.

Amino acid residues of the peptide epitopes that interact with the MHC's groove or pocket may be modified to increase the affinity between the peptide epitope and the MHC binding pocket. Alternatively, residues at the N-terminal and/or C-terminal end of the epitope may be modified (e.g., have 1-3 aa added to either or both ends and/or be substituted with 1-3 aas) to increase the interaction of the peptide epitope with the MHC components of the CIIC; however, such extensions are not counted as part of the peptide epitope's length or included when calculating the epitope's percent identity with another sequence. Such peptide epitopes may be referred to as being “anchor-modified” or N-terminal or C-terminal extended.

T1D or celiac peptide epitopes present in anchor-modified peptide epitopes employed in a CIIC of the present disclosure may comprise a sequence of aas from a T1D- or celiac-associated antigen having a length of, for example, 5 aas to about 25 aas, (e.g., 5 aas to about 7 aas, about 8 aas to about 11 aas, 10 aas to about 15 aas, about 15 aas to about 20 aas, or about 20 aas to about 25 aas) that can bind and interact with, for example, peptide-binding register positions P1-P9, although as indicated above the peptide epitope may be longer. See, e.g., Tollefsen et al., J Clin Invest. 116(8):2226-2236 (2006), doi.org/10.1172/JCl27620). Positions P4, P6, and/or P7 may be modified, and/or the sequence may be extended by one or more, two or more, or three or more aas, on either the N-terminus or C-terminus of the T1D- or celiac-associated antigen sequence.

### a) Peptide Epitopes in CIICs

Among the peptide epitopes that may be bound and presented to a TCR by a CIIC are peptide epitope presenting peptides derived from a variety of self- and non-self-antigens, particularly those associated with specific diseases or disorders. Peptide epitopes of self- and non-self-antigens that may be incorporated into a CIIC include, but are not limited to, peptide epitopes associated with autoantigens, neoantigens, allergens, and antigens derived from infectious agents (e.g., bacteria, viruses, etc.) Such peptide epitopes may be incorporated into CIICs for the treatment or prophylaxis of, for example, autoimmune diseases, cancers, allergies, and viral or bacterial diseases. Peptide epitopes associated with graft versus host disease (“GVHD”) or host versus graft disease (“HVGD”) may also be incorporated into CIICs for the treatment of those conditions. Self-antigens (autoantigens) may be incorporated into CIICs for the treatment or prophylaxis of, for example, autoimmune diseases or disorders other than, or in addition to, T1D and/or celiac disease. Peptide epitopes for the treatment of T1D and/or celiac disease may also be incorporated into the CIICs of the present disclosure.

### (1) Self-Epitopes

In some cases, the peptide epitope of a CIIC is a peptide epitope associated with or present in a self-antigen (an autoantigen). Antigens associated with autoimmune diseases, including those set forth in FIG. 17, can be associated with autoimmune diseases such as Addison disease (autoimmune adrenalitis, Morbus Addison), alopecia areata, Addison's anemia (Morbus Biermer), autoimmune hemolytic anemia (AIHA), autoimmune hemolytic anemia (AIHA) of the cold type (cold hemagglutinin disease, cold autoimmune hemolytic anemia (AIHA) (cold agglutinin disease), (CHAD)), autoimmune hemolytic anemia (AIHA) of the warm type (warm AIHA, warm autoimmune hemolytic anemia (AIHA)), autoimmune hemolytic Donath-Landsteiner anemia (paroxysmal cold hemoglobinuria), antiphospholipid syndrome (APS), atherosclerosis, autoimmune arthritis, arteriitis temporalis, Takayasu arteriitis (Takayasu's disease, aortic arch disease), temporal arteriitis/giant cell arteriitis, autoimmune chronic gastritis, autoimmune infertility, autoimmune inner ear disease (AIED), Basedow's disease (Morbus Basedow), Bechterew's disease (Morbus Bechterew, ankylosing spondylitis, spondylitis ankylosans), Behcet's syndrome (Morbus Behcet), bowel disease including autoimmune inflammatory bowel disease (including colitis ulcerosa (Morbus Crohn, Crohn's disease), autoimmune cardiomyopathy, idiopathic dilated cardiomyopathy (DCM), chronic fatigue immune dysfunction syndrome (CFIDS), chronic inflammatory demyelinating polyneuropathy (CIDP), chronic polyarthritis, Churg-Strauss syndrome, cicatricial pemphigoid, Cogan syndrome, CREST syndrome (syndrome with Calcinosis cutis, Raynaud phenomenon, motility disorders of the esophagus, sklerodaktylia and teleangiectasia), Crohn's disease (Morbus Crohn, colitis ulcerosa), dermatitis herpetiformis during, dermatologic autoimmune diseases, dermatomyositis, essential mixed cryoglobulinemia, fibromyalgia, fibromyositis, Goodpasture syndrome (anti-GBM mediated glomerulonephritis), Guillain-Barre syndrome (GBM, Polyradikuloneuritis), hematologic autoimmune diseases, Hashimoto thyroiditis, hemophilia, acquired hemophilia, autoimmune hepatitis, idiopathic pulmonary fibrosis (IPF), idiopathic thrombocytopenic purpura, Immuno-thrombocytopenic purpura (Morbus Werlhof, ITP), IgA nephropathy, autoimmune infertility, juvenile rheumatoid arthritis (Morbus Still, Still syndrome), Lambert-Eaton syndrome, systemic lupus erythematosus (SLE), lupus erythematosus (discoid form), Lyme arthritis (Lyme disease, borrelia arthritis), Meniere's disease (Morbus Meniere), mixed connective tissue disease (MCTD), multiple sclerosis (MS, encephalomyelitis disseminate, Charcot's disease), myasthenia gravis (myasthenia, MG), myositis, polymyositis, neural autoimmune diseases, pemphigus vulgaris, bullous pemphigoid, polyglandular (autoimmune) syndrome (PGA syndrome, Schmidt's syndrome), polymyalgia rheumatica, primary agammaglobulinemia, primary autoimmune cholangitis, progressive systemic sclerosis (PSS), rheumatoid arthritis (RA, chronic polyarthritis, rheumatic disease of the joints, rheumatic fever), sarcoidosis (Morbus Boeck, Besnier-Boeck-Schaumann disease), stiff-man syndrome, Sclerodermia, Scleroderma, Sjögren's syndrome, autoimmune uveitis, and Wegner's disease (Morbus Wegner, Wegner's granulomatosis).

In some cases, a peptide epitope present in a CIIC is a peptide associated with Addison's disease, alopecia areata, ankylosing spondylitis, autoimmune encephalomyelitis, autoimmune hemolytic anemia, autoimmune hepatitis, autoimmune-associated infertility, autoimmune thrombocytopenic purpura, bullous pemphigoid, Crohn's disease, Goodpasture's syndrome, glomerulonephritis (e.g., crescentic glomerulonephritis, proliferative glomerulonephritis), Grave's disease, Hashimoto's thyroiditis, mixed connective tissue disease, multiple sclerosis, myasthenia gravis (MG), pemphigus (e.g., pemphigus vulgaris), pernicious anemia, polymyositis, psoriasis, psoriatic arthritis, rheumatoid arthritis, scleroderma, Sjögren's syndrome, systemic lupus erythematosus (SLE), vasculitis, or vitiligo.

Autoantigens include, e.g., aggrecan, alanyl-tRNA synthetase (PL-12), alpha beta crystallin, alpha fodrin (Sptan 1), alpha-actinin, α1 antichymotrypsin, α1 antitrypsin, α1 microglobulin, aldolase, aminoacyl-tRNA synthetase, an amyloid, an annexin, an apolipoprotein, aquaporin, bactericidal/permeability-increasing protein (BPI), β-globin precursor BP1, β-actin, β-lactoglobulin A, β-2-glycoprotein I, β2-microglobulin, a blood group antigen, C reactive protein (CRP), calmodulin, calreticulin, cardiolipin, catalase, cathepsin B, a centromere protein, chondroitin sulfate, chromatin, collagen, a complement component, cytochrome C, cytochrome P450 2D6, cytokeratin, decorin, dermatan sulfate, DNA topoisomerase I, elastin, Epstein-Barr nuclear antigen 1 (EBNA1), elastin, entactin, an extractable nuclear antigen, Factor I, Factor P, Factor B, Factor D, Factor H, Factor X, fibrinogen, fibronectin, formiminotransferase cyclodeaminase (LC-1), gp210 nuclear envelope protein, GP2 (major zymogen granule membrane glycoprotein), glycoprotein gpllb/Illa, glial fibrillary acidic protein (GFAP), glycated albumin, glyceraldehyde 3-phosphate dehydrogenase (GAPDH), haptoglobin Δ2, heat shock proteins, hemocyanin, heparin, a histone, histidyl-tRNA synthetase (Jo-1), a hordein, hyaluronidase, immunoglobulins, an integrin, interstitial retinol-binding protein 3, intrinsic factor, Ku (p70/p80), lactate dehydrogenase, laminin, liver cytosol antigen type 1 (LC1), liver/kidney microsomal antigen 1 (LKM1), lysozyme, melanoma differentiation-associated protein 5 (MDAS), Mi-2 (chromodomain helicase DNA binding protein 4), a mitochondrial protein, muscarinic receptors, myelin-associated glycoprotein, myosin, myelin basic protein, myelin proteolipid protein, myelin oligodendrocyte glycoprotein, myeloperoxidase (MPO), rheumatoid factor (IgM anti-IgG), neuron-specific enolase, nicotinic acetylcholine receptor A chain, nucleolin, a nucleoporin, nucleosome antigen, PM/Scl100, PM/Scl 75, pancreatic β-cell antigen, pepsinogen, peroxiredoxin 1, phosphoglucose isomerase, phospholipids, phosphatidyl inositol, platelet derived growth factors, polymerase beta (POLB), potassium channel KIR4.1, proliferating cell nuclear antigen (PCNA), proteinase-3, proteolipid protein, proteoglycan, prothrombin, recoverin, rhodopsin, ribonuclease, a ribonucleoprotein, ribosomes, a ribosomal phosphoprotein, RNA, an Sm protein, Sp100 nuclear protein, SRP54 (signal recognition particle 54 kDa), a selectin, smooth muscle proteins, sphingomyelin, streptococcal antigens, superoxide dismutase, synovial joint proteins, T1F1 gamma collagen, threonyl-tRNA synthetase (PL-7), tissue transglutaminase, thyroid peroxidase, thyroglobulin, thyroid stimulating hormone receptor, transferrin, triosephosphate isomerase, tubulin, tumor necrosis factor-alpha, topoisomerase, U1-dnRNP 68/70 kDa, U1-snRNP A, U1-snRNP C, U-snRNP B/B′, ubiquitin, vascular endothelial growth factor, vimentin, and vitronectin.

Autoantigens associated with alopecia areata (autoimmune alopecia) include, e.g., hair follicle keratinocyte polypeptides, melanogenesis-associated autoantigens, and melanocyte polypeptides. An example of a melanocyte autoantigen is tyrosinase. Autoantigens associated with autoimmune alopecia also include trichohyalin (Leung et al. (2010) J. Proteome Res. 9:5153) and keratin 16. A suitable peptide epitope for inclusion in a CIIC can be a peptide epitope of from 4 aas to about 25 aas in length of a hair follicle keratinocyte polypeptide, a melanocyte polypeptide, a melanogenesis-associated polypeptide, tyrosinase, trichohyalin, or keratin 16.

Autoantigens associated with Addison's disease include, e.g., 21-hydroxylase. A suitable peptide epitope for inclusion in a CIIC can be a peptide epitope of from 4 aas to about 25 aas in length of 21-hydroxylase.

Autoantigens associated with autoimmune thyroiditis (Hashimoto's thyroiditis) include, e.g., thyroglobulin, thyroid peroxidase, thyroid Stimulating Hormone Receptor (TSH-Receptor), thyroidal iodide transporters Na+/I symporter (NIS), pendrin, and the like. A suitable peptide epitope for inclusion in a CIIC can be a peptide epitope of from 4 aas to about 25 aas in length of any one of the aforementioned Hashimoto's thyroiditis-associated polypeptides.

Autoantigens associated with Crohn's disease include, e.g., pancreatic secretory granule membrane glycoprotein-2 (GP2). A suitable peptide epitope for inclusion in a CIIC can be a peptide epitope of from 4 aas to about 25 aas, e.g., from 8 to 25 aas, from 10-20 aas, from 10-15 aas, and from 15-25 aas in length of GP2.

Autoantigens associated with Goodpasture's disease include, e.g., the α3 chain of type IV collagen, e.g., aas 135-145 of the α3 chain of type IV collagen. Penades et al. (1995) Eur. J. Biochem. 229:754, Kalluri et al. (1994) Proc. Natl. Acad. Sci. USA 91:6201. A suitable peptide epitope for inclusion in a CIIC can be a peptide epitope of from 4 aas to about 25 aas, e.g., from 8 to 25 aas, from 10-20 aas, from 10-15 aas, and from 15-25 aas in length of the α3 chain of type IV collagen.

Autoantigens associated with Grave's disease include, for example, thyroglobulin, thyroid peroxidase, and thyrotropin receptor (TSH-R). A suitable peptide epitope for inclusion in a CIIC can be a peptide epitope of from 4 aas to about 25 aas, e.g., from 8 to 25 aas, from 10-20 aas, from 10-15 aas, and from 15-25 aas in length of any one of the aforementioned Grave's disease-associated antigens.

Autoantigens associated with mixed connective tissue disease include, e.g., U1 ribonucleoprotein (U1-RNP) polypeptide (also known as snRNP70). Sato et al. (2010) Mol. Cell. Biochem. 106:55. A suitable peptide epitope for inclusion in a CIIC can be a peptide epitope of from 4 aas to about 25 aas, e.g., from 8 to 25 aas, from 10-20 aas, from 10-15 aas, and from 15-25 aas in length of U1-RNP polypeptide.

Autoantigens associated with multiple sclerosis include, e.g., myelin basic protein, myelin oligodendrocyte glycoprotein, and myelin proteolipid protein. A suitable peptide epitope for inclusion in a CIIC can be a peptide epitope of from 4 aas to about 25 aas, e.g., from 8 to 25 aas, from 10-20 aas, from 10-15 aas, and from 15-25 aas in length of any one of the aforementioned multiple sclerosis-associated antigens. As one non-limiting example, the peptide epitope can comprise the aa sequence ENPVVHFFKNIVTPR (SEQ ID NO:105). In some cases, a CIIC comprises a DRB1*15:01 MHC class II β chain, and a peptide epitope of SEQ ID ID NO:105.

Autoantigens associated with myasthenia gravis include, e.g., acetylcholine receptor (AchR, see, e.g., Lindstrom (2000) Muscle & Nerve 23:453), muscle-specific tyrosine kinase, and low-density lipoprotein receptor-related protein-4. A suitable peptide epitope for inclusion in a CIIC can be a peptide epitope of from 4 aas to about 25 aas, e.g., from 8 to 25 aas, from 10-20 aas, from 10-15 aas, and from 15-25 aas in length of any one of the aforementioned myasthenia gravis-associated antigens. In some cases, a suitable peptide epitope for inclusion in a CIIC is a peptide epitope of from 4 aas to about 25 aas, e.g., from 8 to 25 aas, from 10-20 aas, from 10-15 aas, and from 15-25 aas in length of an AchR.

Autoantigens associated with Parkinson's disease include, e.g., α-synuclein. A suitable peptide epitope for inclusion in a CIIC can be a peptide epitope of from 4 aas to about 25 aas, e.g., from 8 to 25 aas, from 10-20 aas, from 10-15 aas, and from 15-25 aas in length of α-synuclein. For example, a suitable peptide epitope for inclusion in a CIIC includes a peptide of from 5 aas to the entire length of any one of the following: GKTKEGVLYVGSKTK (SEQ ID ID NO:106), KTKEGVLYVGSKTKE (SEQ ID ID NO:107), MPVDPDNEAYEMPSE (SEQ ID ID NO:108), DNEAYEMPSEEGYQD (SEQ ID ID NO:109), EMPSEEGYQDYEPE (SEQ ID ID NO:110), and SEEGYQDYEPEA (SEQ ID ID NO:111) where “S” denotes phosphoserine in those peptides.

Autoantigens associated with pemphigus (e.g., pemphigus vulgaris, pemphigus foliaceus, bullous pemphigoid) include pemphigus vulgaris immunogens such as desmosomal cadherin desmoglein 3 (Dsg3), pemphigus foliaceus immunogens such as Dsg1, bullous pemphigoid immunogens such as hemidesmosome peptides including BP230 antigen, GPAG1a, and BPAG1b. See, e.g., Cirillo et al. (2007) Immunology 121:377.

Autoantigens associated with bullous pemphigoid include bullous pemphigoid antigen 1 (BPAG1, also known as BP230 or dystonin), bullous pemphigoid antigen 2 (BPAG2, also known as BP180 or type XVII collagen), and subunits of human integrins α-5 and β-4. A suitable peptide epitope for inclusion in a CIIC can be a peptide epitope of from 4 aas to about 25 aas, e.g., from 8 to 25 aas, from 10-20 aas, from 10-15 aas, and from 15-25 aas in length of any of the aforementioned pemphigus-associated antigens.

Autoantigens associated with myositis (e.g., polymyositis, dermatomyositis) include, e.g., histidyl tRNA synthetase. A suitable peptide epitope for inclusion in a CIIC can be a peptide epitope of from 4 aas to about 25 aas, e.g., from 8 to 25 aas, from 10-20 aas, from 10-15 aas, and from 15-25 aas in length of histidyl tRNA synthetase.

Autoantigens associated with rheumatoid arthritis include, e.g., collagen, vimentin, aggrecan, fibrinogen, cyclic citrullinated peptides, α-enolase, histone polypeptides, lactoferrin, catalase, actinin, and actins (cytoplasmic 1 and 2(β/γ)). A suitable peptide epitope for inclusion in a CIIC can be a peptide epitope of from 4 aas to about 25 aas, e.g., from 8 to 25 aas, from 10-20 aas, from 10-15 aas, and from 15-25 aas in length of any one of the aforementioned rheumatoid arthritis-associated antigens.

Autoantigens associated with scleroderma include nuclear antigens. A suitable peptide epitope for inclusion in a CIIC can be a peptide epitope of from 4 aas to about 25 aas, e.g., from 8 to 25 aas, from 10-20 aas, from 10-15 aas, and from 15-25 aas in length of a nuclear antigen associated with scleroderma.

Autoantigens associated with Sjögren's syndrome include, e.g., Ro/La ribonucleoprotein (RNP) complex, alpha-fodrin, beta-fodrin, islet cell autoantigen, poly(ADP)ribose polymerase (PARP), nuclear mitotic apparatus (NuMA), NOR-90, Ro60 kD autoantigen, Ro52 antigen, Lα antigen (see, e.g., GenBank Accession No. NP_001281074.1), and p27 antigen. A suitable peptide epitope for inclusion in a CIIC can be a peptide epitope of from 4 aas to about 25 aas, e.g., from 8 to 25 aas, from 10-20 aas, from 10-15 aas, and from 15-25 aas in length of any one of the aforementioned Sjögren's syndrome-associated antigens.

Autoantigens associated with systemic lupus erythematosus (SLE) include, e.g., Ro60 autoantigen, low-density lipoproteins, Sm antigens of the U-1 small nuclear ribonucleoprotein complex (B/B′, D1, D2, D3, E, F, G), α-actin 1, α-actin 4, annexin Al , Clq/tumor necrosis factor-related protein, catalase, defensins, chromatin, histone proteins, transketolase, hCAP18/LL37, and ribonucleoproteins (RNPs). A suitable peptide epitope for inclusion in a CIIC can be a peptide epitope of from 4 aas to about 25 aas, e.g., from 8 to 25 aas, from 10-20 aas, from 10-15 aas, and from 15-25 aas in length of any one of the aforementioned SLE-associated antigens.

Autoantigens associated with thrombocytopenia purpura include ADAMTS13 (a disintegrin and metalloproteinase with a thrombospondin type 1 motif, member 13), and von Willebrand factor-cleaving protease (VWFCP). A suitable peptide epitope for inclusion in a CIIC can be a peptide epitope of from 4 aas to about 25 aas, e.g., from 8 to 25 aas, from 10-20 aas, from 10-15 aas, and from 15-25 aas in length of an ADAMTS13 polypeptide or a VWFCP polypeptide.

Autoantigens associated with vasculitis include proteinase-3, lysozyme C, lactoferrin, leukocyte elastase, cathepsin G, and azurocidin. A suitable peptide epitope for inclusion in a CIIC can be a peptide epitope of from 4 aas to about 25 aas, e.g., from 8 to 25 aas, from 10-20 aas, from 10-15 aas, and from 15-25 aas in length of any of the aforementioned vasculitis-associated antigens.

Autoantigens associated with vitiligo include SOX9, SOX10, PMEL (Premelanosomal protein), tyrosinase, TYRP1 (Tyrosine related protein 1), DDT (D-Dopachrome tautomerase), Rab38, and MCHR1 (Melanin-concentrating receptor. A suitable peptide epitope for inclusion in a CIIC can be a peptide epitope of from 4 aas to about 25 aas, e.g., from 8 to 25 aas, from 10-20 aas, from 10-15 aas, and from 15-25 aas in length of any one of the aforementioned vitiligo-associated polypeptides.

Autoantigens associated with autoimmune uveitis include, for example, interphotoreceptor retinoid-binding protein (IRBP, also known retinol binding protein 3). A suitable peptide epitope for inclusion in a CIIC can be a peptide epitope of from 4 aas to about 25 aas, e.g., from 8 to 25 aas, from 10-20 aas, from 10-15 aas, and from 15-25 aas in length of IRBP. A suitable peptide epitope for inclusion in a CIIC can be a peptide epitope of from 4 aas to about 25 aas, e.g., from 8 to 25 aas, from 10-20 aas, from 10-15 aas or from 15-25 aas in length of any one of the aforementioned antigens.

Autoantigens associated with autoimmune polyendocrine syndrome include, e.g., 17-alpha hydroxylase, histidine decarboxylase, tryptophan hydroxylase, and tyrosine hydroxylase. A suitable peptide epitope for inclusion in a CIIC can be a peptide epitope of from 4 aas to about 25 aas, e.g., from 8 to 25 aas, from 10-20 aas, from 10-15 aas, and from 15-25 aas in length of any one of the aforementioned autoimmune polyendocrine syndrome-associated antigens.

Autoantigens associated with psoriasis include ADAMTS15. See, e.g., Prinz (2017) Autoimmunity Reviews 16:970. A suitable peptide epitope for inclusion in a CIIC can be a peptide epitope of from 4 aas to about 25 aas, e.g., from 8 to 25 aas, from 10-20 aas, from 10-15 aas, or from 15 to 25 aas in length of an ADAMTS15 polypeptide.

(2) T1D-Associated Antigens and their Epitopes

Antigens associated with type 1 diabetes (T1D-associated antigens) include, e.g., preproinsulin, proinsulin, insulin, insulin B chain, insulin A chain, proinsulin C-peptide, 65 kDa isoform of glutamic acid decarboxylase (GAD65), 67 kDa isoform of glutamic acid decarboxylase (GAD67), tyrosine phosphatase (IA-2), heat-shock protein HSP65, islet-specific glucose-6-phosphatase catalytic subunit related protein (IGRP), islet antigen 2 (IA2), and zinc transporter (ZnT8). See, e.g., Mallone et al. (2011) Clin. Dev. Immunol. 2011:513210; and U.S. Patent Publication No. 2017/0045529. A suitable T1D-epitope for inclusion in a CIIC can be a peptide epitope of from 4 aas to about 25 aas in length (or of any length within that range, e.g., from 4 aas to 10 aas, from 10 aas to 15 aas, from 10 aas to 20 aas, or from 15 aas to 25 aas) of any one of the above-mentioned T1D-associated antigens.

As one non-limiting example, a T1D-epitope is proinsulin 73-90 (GAGSLQPLALEGSLQKR; SEQ ID NO:177). As another non-limiting example, a T1D-epitope is the insulin (InsA (1-15)) peptide: GIVDQCCTSICSLYQ (SEQ ID NO:178). As another non-limiting example, a T1D-epitope is the insulin (InsA 1-15, D4E) peptide: GIVEQCCTSICSLYQ (SEQ ID NO:327). As another non-limiting example, a T1D-epitope is the GAD65 (555-567) peptide, NFFRMVISNPAAT (SEQ ID NO:280). As another non-limiting example, a T1D-epitope is the GAD65 (555-567, F5571) peptide, NFIRMVISNPAAT (SEQ ID NO:280). As another non-limiting example, a T1D-epitope is the islet antigen 2 (IA2) peptide: SFYLKNVQTQETRTLTQFHF (SEQ ID NO:180). As another non-limiting example, a T1D-epitope is the proinsulin peptide: SLQPLALEGSLQSRG (SEQ ID NO:281). As another non-limiting example, a T1D-epitope is the proinsulin peptide GSLQPLALEGSLQSRGIV (SEQ ID NO:282, prolns 75-92(K88S)).

In some cases, the peptide epitope comprises from 4 to about 25 contiguous aas of an aa sequence having at least 90%, at least 95%, at least 98%, at least 99%, or 100% aa sequence identity to aas 25-110 of the human preproinsulin aa sequence (wherein aas 1-24, bolded and italicized, form the signal peptide): MALWMRLLPL LALLALWGPD PAAAFVNQHL CGSHLVEALY LVCGERGFFY TPKTRREAED LQVGQVELGG GPGAGSLQPL ALEGSLQKRG IVEQCCTSIC SLYQLENYCN (SEQ ID NO:283), where the T1D-epitope has a length of 4 aas (aa), 5 aas, 6 aas, 7, aas, 8 aas, 9 aas, 10 aas, 11 aas, 12 aas, 13 aas, 14 aas, 15 aas, 16 aas, 17 aas, 18 aas, 19 aas, 20 aas, 21 aas, 22 aas, 23 aas, 24 aas, or 25 aas. In some cases, the peptide epitope may have the aa sequence: GAGSLQPLALEGSLQKRG (SEQ ID NO:284). A T1D-epitope may have the aa sequence: SLQPLALEGSLQKRG (SEQ ID NO:285). A T1D-epitope may have the aa sequence: SLQPLALEGSLQSRG (SEQ ID NO:281, PROINS 76-90 (K88S)). A T1D-epitope may have the aa sequence: QPLALEGSLQKRG (SEQ ID NO:286). A T1D-epitope may have the aa sequence: QPLALEGSLQSRG (SEQ ID NO:287). As another non-limiting example, a T1D-epitope is the human proinsulin peptide, GSLQPLALEGSLQSRGIV (SEQ ID NO:282, prolns 75-92 (K88S)).

(3) Celiac Disease-Associated Antigens and their Epitopes

Antigens associated with celiac disease (celiac disease-associated antigens) include, e.g., tissue transglutaminase and gliadin. Other celiac disease-associated antigens include, e.g., secalins, hordeins, avenins, and glutenins. Examples of secalins include rye secalins. Examples of hordeins include barley hordeins. Examples of glutenins include wheat glutenins. See, e.g., U.S. 2016/0279233. A suitable celiac-epitope for inclusion in a CIIC can be a peptide epitope of from 4 aas to about 25 aas in length (e.g., about 5 to about 25) or of any length within that range (e.g., from 4 aas to 10 aas, from 10 aas to 15 aas, from 10 aas to 20 aas, or from 15 aas to 25 aas) of any one of the above-mentioned celiac disease-associated antigens.

For example, a suitable celiac-associated peptide epitope is in some cases a peptide of from about 4 to about 25 contiguous aas (or of any length within that range, e.g., from 4 aas to 10 aas, from 10 aas to 15 aas, from 10 aas to 20 aas, or from 15 aas to 25 aas) of a polypeptide comprising an aa sequence having at least about 85%, at least about 90%, at least about 95%, at least about 98%, at least about 99%, or 100% aa sequence identity to the following gamma-gliadin aa sequence: MKTLLILTIL AMATTIATAN MQVDPSGQVQ WPQQQPFPQP QQPFCEQPQR TIPQPHQTFH HQPQQTFPQP EQTYPHQPQQ QFPQTQQPQQ PFPQPQQTFP QQPQLPFPQQ PQQPFPQPQQ PQQPFPQSQQ PQQPFPQPQQ QFPQPQQPQQ SFPQQQQPLI QPYLQQQMNP CKNYLLQQCN PVSLVSSLVS MILPRSDCKV MRQQCCQQLA QIPQQLQCAA IHGIVHSIIM QQEQQQQQQQ QQQQQQQQGI QIMRPLFQLV QGQGIIQPQQ PAQLEVIRSL VLGTLPTMCN VFVPPECSTT KAPFASIVAD IGGQ (SEQ ID NO:288). In some cases, the celiac-epitope is a Glia-α9 epitope. Glia-α9 is a major (immunodominant) epitope that is recognized by the majority of celiac disease (CD) patients. Glia-α9 epitopes include, e.g., QPFPQPQ (SEQ ID NO:289), and PFPQPQLPY (SEQ ID NO:290), which when selectively deamidated by transglutaminase 2 and presented by HLA-DQ2 as the amino-acid sequence PFPQPELPY (SEQ ID NO:291) induces potent T-cell responses. The celiac-epitope may comprise a sequence selected from a gliadin alpha1a peptide QLQPFPQPELPY (SEQ ID NO:175), or LQPFPQPELPY (SEQ ID NO:292). The gliadin epitope may also comprise a C-terminal or N-terminal extended and/or anchor-modified gliadin alpha1a peptide (e.g., for expression enhancement) selected from: ADAQLQPFPQPELPY (SEQ ID NO:293), ADALQPFPQPELPY (SEQ ID NO:294), ADAQPFPQPELPY (SEQ ID NO:295), ADAPFPQPELPY (SEQ ID NO:296), QLQIFPQPELPY (SEQ ID NO:297), QLQPFPEPELPY (SEQ ID NO:298), QLQPFPQPEEPY (SEQ ID NO:299), and QLQIFPEPEEPY (SEQ ID NO:186). The celiac-epitope may comprise a gliadin alpha 2 peptide sequence selected from PQPELPYPQPE (SEQ ID NO:177) and QPQPELPYPQPE (SEQ ID NO:187). The gliadin epitope may also comprise an N-terminal extended and/or anchor-modified gliadin alpha 2 peptide (e.g., for expression enhancement) selected from: ADAQPQPELPYPQPE (SEQ ID NO:300), ADAPQPELPYPQPE (SEQ ID NO:301), IQPELPYPQPE (SEQ ID NO:302), PQPELPEPQPE (SEQ ID NO:303), IQPELPEPQPE (SEQ ID NO:188) PFPQPELPYPQPE (SEQ ID NO:304), QPFPQPELPYPQPE (SEQ ID NO:176), FPQPELPYPQPE (SEQ ID NO:306), and APQPELPYPQPE (SEQ ID NO:307). The celiac-epitope may comprise a gliadin omega peptide selected from QPFPQPEQPFPW (SEQ ID NO:308), QPEPFPQPEQPFPW (SEQ ID NO:309), PEPFPQPEQPFPW (SEQ ID NO:310), EPFPQPEQPFPW (SEQ ID NO:311), and PFPQPEQPFPW (SEQ ID NO:312).

In some cases, the celiac-epitope is a gliadin epitope presenting peptide modified for expression enhancement and contains a cysteine for anchoring the peptide in the binding groove. For example, the peptide may comprise the cysteine substituted alpha 1a gliadin peptide sequence QLQPFPQPCLPY (SEQ ID NO:313) or the alpha 2 gliadin peptide sequence PQPELCYPQPE (SEQ ID NO:314).

### (4) Epitopes of Allergens

In some cases, the epitope peptide presented in the context of a CIIC comprises an epitope of an allergen. Allergens are too numerous to recite, but by way of example, allergens include, but are not limited to, peanuts and tree nuts, plant pollens, and the like. Allergens also include proteins from hymenoptera proteins (e.g., allergens in bee and wasp venoms such as phospholipase Δ2, melittin, “antigen 5” found in wasp venom, and hyaluronidases).

Peptide epitopes of peanut allergens, such as the Ara h 1 to 13 proteins may come from, for example, seven protein families, include those in Ara h 1 (e.g., PGQFEDFF (SEQ ID NO:328), YLQGFSRN (SEQ ID NO:329), FNAEFNEIRR (SEQ ID NO:330), QEERGQRR (SEQ ID NO:331), DITNPINLRE (SEQ ID NO:332), NNFGKLFEVK (SEQ ID NO:333), GNLELV (SEQ ID NO:334), RRYTARLKEG (SEQ ID NO:335), ELHLLGFGIN (SEQ ID NO:336), HRIFLAGDKD (SEQ ID NO:337), IDQIEKQAKD (SEQ ID NO:338), KDLAFPGSGE (SEQ ID NO:339), KESHFVSARP (SEQ ID NO:340), NEGVIVKVSKEHVEELTKHAKSVSK (SEQ ID NO:341)), Ara h 2 (e.g., HASARQQWEL (SEQ ID NO:342), QWELQGDRRC (SEQ ID NO:343), DRRCQSQLER (SEQ ID NO:344), LRPCEQHLMQ (SEQ ID NO:345), KIQRDEDSYE (SEQ ID NO:346), YERDPYSPSQ (SEQ ID NO:347), SQDPYSPSPY (SEQ ID NO:348), DRLQGRQQEQ (SEQ ID NO:349), KRELRNLPQQ (SEQ ID NO:350), QRCDLDVESG (SEQ ID NO:351)), and Ara h 3 (e.g., IETWNPNNQEFECAG (SEQ ID NO:352), GNIFSGFTPEFLAQA (SEQ ID NO:353), VTVRGGLRILSPDRK (SEQ ID NO:354), DEDEYEYDEEDRRRG (SEQ ID NO:355)). See, e.g., Zhou et al, (2013) Intl. J. ofFood Sci. 2013: 8 pages article ID 909140.

### (5) Pepide Epitopes of Cancer-Associated Antigens

Peptide epitopes of cancer-associated antigens that may be presented by a CIIC of the present disclosure may be derived from, for example, neoantigens, oncogenes (e.g., Wilms' Tumor WT1 protein), Alpha Feto Protein (AFP), viral oncogenes (e.g., E6 and E7 oncogene products from oncogenic strains of HPV), and proteins of viruses such as HBV and HCV, resulting in oncogenesis. A number of such proteins are disclosed in published PCT applications WO2020132138Δ1, WO2019051091 and WO2020132297.

### 7. Additional Polypeptide Sequences

Additional polypeptide sequences that provide any of a variety of functions may be incorporated into the polypeptide chain of a CIIC. The functions of additional peptide sequences include, but are not limited to, providing affinity tags and/or polypeptide affinity domains (e.g., use for purification of CIICs), acting as labels or labeling sites (e.g., useful for identifying the in vitro or in vivo location of CIICs or identifying a T-cell with a cognate TCR that recognizes the epitope presented by the CIIC), targeting CIICs, and as a sequence for post-translational modifications. Additional polypeptide sequences can be present in a variety of locations in a CIIC, including adjacent to, or integrated into, linker sequences (e.g., L3 or L4 linkers), membrane proximal sequences, and scaffold sequences.

The additional polypeptide sequence may be less than about 200 aas (e.g., from about 100 to about 200 aas, or from about 50 about 100 aas). The additional polypeptide sequence may be less than about 50 aas (from about 25 to about 50 aas). In some instances, the additional polypeptide sequence is less than about 25 aas (e.g, from 12-25 aas). In some instances, the additional polypeptide sequence is less than 12 aas (e.g, from 8-12 aas). In some instances, the additional polypeptide sequence is less than 8 aas (e.g, from 2-8 aas).

### a) Affinity Tags

Affinity tags or affinity domains include aa sequences that can interact with a binding partner, e.g., such as one immobilized on a solid support. Affinity tags are useful for identifying the location of CIICs, quantification of CIICs, and their purification. Using affinity tags, the location of a CIIC in a sample (e.g., of target tissue) can be determined by contacting the affinity tag with a labeled binding partner. Where the binding partner for the affinity tag is immobilized (e.g., on a matrix such as a chromatographic matrix), the affinity tag may be used for purification of the CIIC. In addition, immobilization of a CIIC on the surface of a biosensor (e.g., a plasmon resonance sensor) by its affinity domain permits a variety of biochemical assessments to be conducted on the CIIC.

Some suitable affinity tags or affinity domains include, but are not limited to, hemagglutinin (HA, e.g., YPYDVPDYA, SEQ ID NO:315), StrepTag (WSHPQFEK, SEQ ID NO:316), FLAG (e.g., DYKDDDDK, SEQ ID NO:317), c-myc (e.g., EQKLISEEDL, SEQ ID NO:318), RYIRS (SEQ ID NO:319), FHHT (SEQ ID NO:320), WEAAAREACCRECCARA (SEQ ID NO:321), and the like. Some affinity tags suitable for protein purification on immobilized metal matrices include multiple consecutive aas, such as histidine (e.g., HisX5 (HHHHH) (SEQ ID NO:322), or HisX6 (HHHHHH) (SEQ ID NO:323) which, when fused to the expressed protein, may be used for its chromatographic purification by high affinity binding to a chromatographic matrix such as nickel Sepharose®. Other affinity tags include glutathione-S-transferase (GST), thioredoxin, cellulose binding domains, chitin binding domains, S-peptide, T7 peptide, SH2 domains, C-end RNA tag, inteins, biotin, streptavidin, MyoD, leucine zipper sequences, maltose binding protein, and metal binding domains such as zinc binding domains or calcium binding domains (e.g., those from calcium-binding proteins calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D9K, calbindin D28K, calretinin).

Otherwise soluble CIICs bearing an affinity tag may be immobilized by binding with the affinity domain's cognate binding partner. The CIIC may be immobilized using one or more antibodies that recognize the affinity tag (or another part of the CIIC). CIICs may be immobilized on matrices including, but not limited to, chromatography matrices, sensor surfaces, or other solid or semi-solid (e.g., gel) matrices bearing a binding partner specific to the counterpart to the affinity tag.

### b) Targeting Sequences

CIICs may include a targeting polypeptide or “targeting sequence.” Targeting sequences serve to bind or “localize” CIICs to cells and/or tissues displaying the protein (or other molecule) to which the targeting sequence binds. Targeting sequences may be located, for example, at or near the carboxyl terminal end of the α2 domain, or a membrane proximal region or scaffold attached thereto. See, e.g., in FIG. 1, structures A and H, where the targeting sequence may replace one or more of the scaffold, L4 linker, additional polypeptide (“Addn. Pep”), or the entire Scaffold/L4/Addn. Pep structure(s) (with the L3 and L4 linkers being optional). CIICs may comprise both scaffolds and targeting sequences. In some cases, a targeting sequence may be an antibody or portion (e.g., fragment) thereof (e.g., a scFv or a nanobody such as a heavy chain nanobody or a light chain nanobody). Targeting sequences such as antibody Fc domains or nanobodies may also be used to immobilize CIICs to surfaces, or portions of the surfaces, of detectors, cell culture wares, biological arrays and the like. In some cases, a targeting sequence may be a single-chain T cell receptor (scTCR).

Targeting sequences in the form of a CIIC additional polypeptide may be translated as part of the CIIC polypeptide; however, it is also possible to target CIICs using targeting moieties covalently attached (e.g., using a crosslinker) or non-covalently attached (e.g., using a biotin-avidin linkage). When using covalent or non-covalent attachment the targeting moieties essentially become a payload-like molecule attached to the CIIC. Where covalent attachment is employed, it may be through the side chain of an aa (e.g., via a sulfhydryl of a cysteine or the epsilon amine of a lysine). Where non-covalent attachment is employed, the linkage may take a variety of forms. For example, a CIIC having a biotin label polypeptide may be non-covalently attached to an avidin labeled targeting antibody or Fab directed to, for example, an autoantigen). Alternatively, a bispecific antibody (e.g., a bispecific IgG), that may be humanized, having a first antigen binding site directed to a part of the CIIC (e.g., the scaffold sequence) may be employed to non-covalently attach a CIIC to the second bispecific antibody binding site, which acts as a targeting sequence when directed to, for example, a cell or tissue target (e.g., an autoantigen).

Anti-CD4 antibodies and antibody-related molecules (e.g., antigen binding fragments, single chain antibodies, nanobodies, etc.) may be employed to target CIICs to CD4+ T cells. A number of anti-CD4 antibodies including, but not limited to, YTS177, priliximab, keliximab, clenoliximab, zanolimumab, tregalizumab, cedelizumab, and ibalizumab are known. See, e.g., Konig et al., Frontiers in Immunol, Vol. 7 article 11 (2016) (doi: 10.3389/fimmu.2016.00011), see, also, Helling et al., Immunology and Cell Biology 93: 396-405 (2015). Those and other anti-CD4 antibodies may function as CIIC targeting polypeptides or sequences, and also provide the sequences for the construction of antibody-related molecules and sequences that bind to and target CD4. The targeting polypeptide or targeting sequence may be ibalizumab or an antibody-related molecule based upon ibalizumab (e.g., having the antigen binding sequences of ibalizumab).

### c) Post-Translational Modification Sequences

Additional polypeptides that serve as post-translational modification sequences can provide sites for CIIC modifications including addition of carbohydrates and similar molecules (sialic acid), phosphorylation, lipid addition and the like. While post-translational modification sequences may be located anywhere in the CIIC, those sequences, particularly when used for the addition of lipids or a prenyl group, are typically located at or near the carboxyl terminal end of an α2 domain, or a sequence C-terminal to the α2 domain such as a scaffold sequence. More specifically, post-translational modification sequences may be located in, or in or adjacent to, any one or more of the L3 linker, scaffold, and/or L4 linker (e.g., as an additional polypeptide of a CIIC (see, e.g., in FIG. 1, structure A)).

As previously indicated, in some instances post-translational modification sequences (e.g., for lipid or prenyl group addition leading to membrane association) may be integrated into, or located adjacent to, the N-terminus or C-terminus of the scaffold sequence. Using sequences adding hydrophobic moieties, otherwise soluble MHC Class II molecules may be made to associate with lipid bilayers. Such aa sequences may, for example, result in direct or indirect covalent attachment to a lipid or prenyl group. For example, where CIICs are to be associated with a membrane a farnesyltransferase or geranylgeranyl transferase motif may be located at the COOH-terminus of the CIIC. Other prenylation sites such as CC and CXC sites may also be employed in CIICs (see, e.g., Beranger et al, J. Biol. Chem. 269(18) 12637-643 (1994)).

### d) Labeling Sequences

The additional polypeptides of CIICs include labeling sequences capable of acting as detectable labels. Such labels may be peptides/polypeptides that are detectable optically by fluorescence (fluorescent reporter sequences) or optically (e.g., they have a distinct absorption). Alternatively, they may have catalytic activity (enzymatic activity) such as sequences from horseradish peroxidase (HRP). Where the labeling sequences are larger, they may be considered a fusion protein.

Suitable fluorescent polypeptides/proteins include, but are not limited to, green fluorescent protein (GFP) or variants thereof, blue fluorescent variants of GFP (BFP), cyan fluorescent variants of GFP (CFP), yellow fluorescent variants of GFP (YFP), enhanced GFP (EGFP), enhanced CFP (ECFP), enhanced YFP (EYFP), and the like. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods, 2:905-909), and the like. Any of a variety of fluorescent and colored proteins from anthozoan species, as described in, e.g., Matz et al. (1999) Nature Biotechnol., 17:969-973, may also be suitable for use.

Suitable enzymes that may be employed as labels include, but are not limited to, horseradish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

CIIC may also comprise other types of detectable label sequences suitable for use in in vivo imaging, e.g., suitable for use in positron emission tomography (PET), single photon emission tomography (SPECT), near infrared (NIR) optical imaging, x-ray imaging, computer-assisted tomography (CAT), or magnetic resonance imaging (MRI), or other in vivo imaging method. Labels of those types include sequences capable of binding metal ions (e.g., by chelation) which may act as radiolabels or other types of labels.

As an alternative to the incorporation of aa sequences that act as labels, or that can bind labels, chemical groups can act as labels or binding labeling agents can be added to CIICs. Examples of suitable labels for in vivo imaging include gadolinium or indium chelates (see, e.g., indium chelates with DTPA (diethylenetriamine penta-acetic acid), Arano et al J. Med. Chem. 39, 3451-3460(1996)), DTPA-bismethylamide (BMA), DOTA (dodecane tetraacetic acid), or HP-DO3A (1,4,7-tris(carboxymethyl)-10-(2′-hydroxypropyl)-1,4,7,10-tetraazacyclododecane)), iron chelates, magnesium chelates, manganese chelates, copper chelates, chromium chelates, iodine-based materials, and radionuclides.

Because the CIICs described herein comprise a peptide epitope and the Class II MHC sequences necessary for epitope presentation to a TCR, the CIICs can specifically interact with T-cells bearing a TCR specific/selective for the epitope. Accordingly, when labeled, the CIIC may be used to identify or locate T cells with TCRs specific to the epitope. As such, the present disclosure provides a method of detecting an antigen-specific T-cell. The method comprises contacting a T cell with a CIIC (e.g., a labeled CIIC) and detecting binding of the CIIC to the T cell, either directly or indirectly through detection of the label. Binding of CIICs to the T cell indicates that the T cell is specific for the epitope presented by the CIIC. As noted above, suitable detectable labels include, but are not limited to, a radioisotope, a fluorescent polypeptide, or an enzyme that generates a colored, luminescent, or fluorescent product. In some cases, the T cell being detected is present in a sample comprising a plurality of T cells. For example, a T cell being detected can be present in a sample comprising from 10 to 109 T cells, e.g., from 10 to 10², from 10² to 104, from 104 to 10⁶, from 10⁶ to 107, from 107 to 10⁸, from 10⁸ to 109, or more than 109 T cells.

### 8. Payload-Drug and Other Conjugates

A CIIC can comprise a payload such as a therapeutic (e.g., a small molecule drug or therapeutic), a label (e.g., a fluorescent label or radio label), or other biologically active agent that is linked (e.g., covalently attached) to the polypeptide chain. For example, where a CIIC comprises an Fc polypeptide sequence, that sequence may comprise a covalently linked payload such as an agent that treats an autoimmune disease, potentiates the action of the CIIC, or is an agent that relieves a symptom of a disease.

A payload can be linked directly or indirectly to a polypeptide chain of a CIIC (e.g., to an Ig Fc polypeptide in the CIIC). Direct linkage can involve linkage to an aa (e.g., at a side chain) without an intervening linker. Indirect linkage can arise via a cross-linker, such as a bifunctional cross-linker. Any acceptable chemical linkage may be used including, but not limited to, a thioether bond, an amide bond, a carbamate bond, a disulfide bond, or an ether bond, including those formed by reaction with a crosslinking agent.

Suitable payloads (e.g., drugs) include virtually any small molecule (e.g., less than 2,000 Daltons in molecular weight) approved by the U.S. Food and Drug Administration, and/or listed in the 2020 U.S. Pharmacopeia or National Formulary. In an embodiment, those drugs are less than about 2,000 Da molecular weight. Suitable drugs include non-steroidal anti-inflammatory drugs (NSAID), glucocorticoids, and the like.

C. Stabilization, expression, and properties

Modifications to MHC class II heterodimers that result in CIICs capable of increased expression, i.e., relative to CIICs that do not possess such modifications, and stabilize the expressed CIICs include formatting the molecules as a single polypeptide comprising sequences of both MHC Class II α and β chain (subunit) sequences, an L1 linker aa sequence, and a peptide epitope. By associating the epitope and the α and β subunit sequences, the single chain format overcomes weak interactions and promotes proper folding and association of those components. The single polypeptide chain may be stabilized by one or more disulfide bonds and one or more aa substitutions. FIGS. 13 and 14 indicate the location of some aas that may be used in body disulfide bond and linker disulfide bond formation in various HLA gene products, and can be used to identify aas at corresponding aa positions in alleles related to those shown. A body disulfide is shown schematically in FIG. 1 as a dashed line below, for example, construct A. Linker disulfide bonds are shown schematically in FIG. 1 as a dashed line above, for example, constructs E and F.

As indicated above, additional stabilization may be obtained by introducing aa substitutions that improve hydrogen bonding between the α and β subunit sequences, and/or substitutions that enhance peptide HLA binding interactions.

The folding of the MHC (HLA) a and 1 polypeptide sequences of MAPPs expressed in mammalian cells can be assessed by antibodies that bind only to properly folded, but not denatured, MHC sequences. For example, the anti-HLA DQ antibody SPV-L3 (Novus Biologicals Centennial, CO, USA), which recognizes the intact native DQ2.5 aR heterodimer, but not denatured or misfolded DQ2.5 protein, may be used to probe the structure of DQ2.5 containing proteins to indicate that the protein is properly folded.

### 1. Body and Linker Disulfide Bond Stabilization

Both CIIC stabilizing body disulfide and linker disulfide bonds tether a cysteine located in the last 11 aas, including e.g., the last 10 aas, of the MHC α subunit α1 domain sequence to a location either in the N-terminus of the MHC 1 subunit β1 domain or an L1 linker attached to it. CIIC stabilizing body disulfide bonds are formed between a cysteine located at any of aas 1-8 of the β1 domain and a cysteine located in, for example, the C-terminal 5 aas, including e.g., the C-terminal 4 aas, of the α1 domain. For example, a CIIC stabilizing body disulfide may be formed between a cysteine located at one of aas 4-7 (any one of aas 4, 5, 6 or 7 or aas 5-7) of the β1 domain and a cysteine located in the C-terminal 5 aas of the α1 domain (e.g., cysteine substitutions at β1 position 5 and α1 position 83, such as those resulting from E5C and A83C in HLA DQ 2.5). In some instances, position 5 or 7 of the β1 domain may be substituted with a cysteine for formation of a body disulfide bond.

While any of the last 11 aas of the MHC α subunit α1 domain sequence may be substituted with a cysteine to form a CIIC stabilizing linker disulfide bond, typically the α1 domain cysteine of a linker disulfide bond will be at the 7th, 8th, 9th, 10th, or 11th aa from the C-terminus of the α1 domain (e.g., cysteine substituted at 10, 9 or 8 aa residues from the C-terminus of the α1 domain sequence). Cysteines substituted at any of those α1 domain sequence positions may form a disulfide bond with a cysteine substituted in the L1 linker proximal to the peptide epitope sequence. Cysteines may for example be substituted in the first 5 aas of the L1 linker (counted from the N-terminus of the linker to which the epitope is attached). The cysteine may be located within the first 3 aas of the L1 linker. The cysteine may be located at the second aa position of the L1 linker, and where the linker is otherwise made of G₄S (SEQ ID NO:237) or G₃S (SEQ ID NO:238) repeating units, the substitution may be referred to as a G2C substitution.

### 2. Stabilization of CIICs Comprising MHC DQ Gene Sequences

CIICs comprising MHC (HLA) DQ polypeptide sequences may be stabilized by body disulfide or linker disulfide bonds as well as by additional aa substitutions. For CIICs comprising DQ polypeptide sequences, either body or linker disulfides may be utilized to stabilize the CIIC and to provide for increased expression production; however, body disulfides appear to be more effective.

Body disulfide bonds may be formed between a cysteine located in the last 10 or 11 aas of the MHC α1 domain sequence and a cysteine located in the first 8 aas of the CIIC's DQB β1 domain sequence. In particular, a body disulfide may be formed between the last 5 or 4 aas of the α1 domain sequence and positions 4-7 aas of the β1 domain. For example, a body disulfide may be formed between cysteine substituted for an aa in the sequence “TAA” at positions 82-84 or 83-85 of the MHC DQA1 or DQA2 α1 domain sequence (see FIGS. 11 and 12) and a cysteine in the DQB1 β1 domain sequence “PEDF” (SEQ ID NO:197) at positions 4-7 (e.g., a disulfide bond formed between an A83C substitution in the α1 domain and an E5C substitution in the β1 domain). Where CIICs comprise DQB2 alleles, the corresponding β1 domain sequence is PKDFL (SEQ ID NO:198).

Linker disulfide bonds are typically formed between a cysteine positioned at aas 10, 9 or 8 from the C-terminus of the α1 domain in the sequence “IKR” (positions 76-78 or 77-79 depending on the allele depicted in FIG. 11) and a cysteine positioned in the first 5 aas of the L1 linker. For DQA2 the corresponding sequence for cysteine substitution is “MRQ,” and is located at positions 77-79 of the α1 domain sequence (see FIG. 12).

Substitutions that may be employed to stabilize CIICs in the presence or absence of linker or body disulfide bonds include substitutions at any one or more of 40, 47, 52, or 75 of HLA DQA1*01:01 and DQA2*01:01 α1 domain sequences, or the corresponding locations in the α1 domain sequence of other DQA1 or DQA2 alleles (e.g., positions 40, 47, 52, or 75 of DQA1*02:01, DQA1*05:01).

The amino acid at position 47 of the DQA polypeptides appears to be involved in hydrogen bonding with the α2 domain sequence and/or β2 domain sequence. When position 47 is, for example, an unpaired Cys that does not effectively hydrogen bond to those domains (e.g., as in DQA1*05:01), MHC and CIIC expression levels are markedly lower and the CIIC molecules tend to undergo aggregation. Accordingly, expression of CIICs comprising DQ polypeptide sequences may be facilitated by incorporating a nonreactive aa (e.g., other than cysteine) at position 47 capable of engaging the α2 domain sequence and/or β2 domain sequence (e.g., to reduce aggregation) in addition to either a body or linker disulfide bond (see e.g., Example 3). In order to permit the aa at position 47 of DQA1 or DQA2 polypeptides to interact with the α2 domain sequence and/or β2 domain sequence, that position may be substituted by an aa other than Cys, which in some instances may be a Ser or a positively charged amino acid such as a Lys (K) or Arg (R) (C47S, C47R or C47K substitutions). Alternatively, position 47 may be Lys, Arg, or Ser, or may be substituted by a Lys, Arg, or Ser, (e.g., a Lys or Ser). Substitutions at position 47 of the α chain are not limited to CIICs with DQA1*05:01 sequences, which have a cysteine at that position. CIICs comprising the sequences of other DQA alleles, DRA alleles, DPA alleles, or other MHC alleles may comprise a substitution at position 47 (or its corresponding aa position based on sequence alignment) that removes an unpaired cysteine replacing it with a neutral nonreactive amino acid (e.g, serine) and/or a substitution that provides an aa capable of bonding to the α2 domain sequence and/or β2 domain sequence stabilizing the CIIC. Accordingly, substitutions such as C47S, C47R or C47K or the corresponding substitutions in other alleles (e.g., DR alleles) may be employed to limit aggregation and/or to stabilize the CIIC.

Substitutions at positions 40, 52 and/or 74/75 of the α1 domain may enhance the stability of MHC (HLA) peptide interactions and/or HLA stability. Position 40 may be substituted by an acidic residue such as E or D (e.g., an G40E substitution in DQA1*05:01), position 52 may be substituted by an H (e.g., a R52H substitution in DQA1*05:01), and position 74 or 75, may be substituted by an aliphatic aa such as I, L, or V (e.g., a S741 substitution in DQA1*05:01) to enhance stability.

### 3. Stabilization of CIICs Comprising MHC DR Gene Sequences

As with CIICs having DQ polypeptide sequences, CIICs comprising MHC (HLA) DR polypeptide sequences may be stabilized by body disulfide or linker disulfide bonds as well as by additional aa substitutions. For CIICs comprising DR polypeptide sequences, either body or linker disulfides may be utilized to stabilize the CIIC and to provide for increased expression as compared to CIICs that do not have such disulfides; however, linker disulfides appear to be more effective with at least some DR alleles.

Body disulfide bonds may be formed between a cysteine located in the last 10 or 11 aas of MHC DRA α1 domain sequences and a cysteine located in the first 8 aas of the CIIC's DRB β1 domain. In particular, a body disulfide may be formed between the last 5 or 4 aas of the α1 domain sequence and positions 4-8 aas of the β1 domain. For example, a body disulfide may be formed between a cysteine substituted in the sequence “TPI” at positions 80-82 of an MHC DRA α1 domain sequence (see FIG. 4) and a cysteine substituted at one of positions 5-8 in the DRB1, DRB3, DRB4, or DRB5 β1 domain sequence “PRFL” (SEQ ID NO:194) (e.g., a disulfide bond formed between a P81C substitution in the α1 domain and a P5C substitution in the β1 domain).

Linker disulfide bonds are typically formed between a cysteine positioned at aas 10, 9 or 8 from the C-terminus of the α1 domain in the sequence “IKR” (positions 74-76, see FIG. 4) and a cysteine positioned in the first 5 aas of the L1 linker. Accordingly, DR α subunit α1 domain sequences may comprise an 174C, K75C, or R76C substitution for linker disulfide bond formation.

Substitutions that may be employed to stabilize CIICs in the presence or absence of linker or body disulfide bonds include substitutions at any one or more of 37, 44, 49, and 72 of the α1 domain sequence (corresponding to positions 40, 47, 52, and 75 of HLA DQA1*01:01 and DQA2*01:01), or the corresponding locations in the α1 domain sequence of other DRA alleles. Position 44 may be substituted by an aa other than Cys, which in some instances may be a Ser or a positively charged amino acid such as a Lys (K) or Arg (R) (C44S, C44R or C44K substitutions). Alternatively, position 44 may be Lys, Arg, or Ser, or may be substituted by a Lys, Arg, or Ser, (e.g., a Lys or Ser).

Substitutions at position 37, 49, and/or 72 of DR α subunit α1 domain sequences may enhance the stability of MHC (HLA) peptide interactions and/or HLA stability. Position 37 may be substituted by an acidic residue such as E or D (e.g., an Δ37E substitution), position 49 may be substituted by an H (e.g., a G49H substitution), and position 72 (which is an I in DRA*01:01) may be an aliphatic aa such as I, L, or V (e.g., an 172L, or 172V) to enhance stability.

### 4. Stabilization of CIICs Comprising MHC DP Gene Sequences

As discussed above, CIICs comprising MHC (HLA) DP polypeptide sequences may be stabilized by body disulfide or linker disulfide bonds as well as by additional aa substitutions. For CIICs comprising DP polypeptide sequences, either body or linker disulfides may be utilized to stabilize the CIIC and to provide for increased expression as compared to CIICs that do not have such disulfides.

Body disulfide bonds may be formed between a cysteine located in the last 10 or 11 aas of MHC DPA1 α1 domain sequences and a cysteine located in the first 8 aas of the CIICs DPB β1 domain sequence. In particular, a body disulfide may be formed between the last 5 or 4 aas of the α1 domain sequence and positions 4-8 aas of the β1 domain sequence. For example, a body disulfide may be formed between a cysteine substituted in the sequence “TQA” at positions 83-85 of an MHC DPA α1 domain sequence (see FIG. 9) and a cysteine substituted at one of positions 4-8 in the DPB1 β1 domain sequence, for example in the sequence “PENY” (SEQ ID NO:199, see FIGS. 10 and 16) (e.g., a disulfide bond formed between a Q84C substitution in the α1 domain and an E5C substitution in the β1 domain).

Linker disulfide bonds are typically formed between a cysteine positioned at aas 10, 9 or 8 from the C-terminus of the α1 domain in the sequence “IQR” (positions 77-79, see FIG. 9) and a cysteine in the first 5 aas of the L1 linker. Accordingly, DP α subunit α1 domain sequences may comprise an 177C, Q78C, or R79C substitution for linker disulfide bond formation.

Substitutions that may be employed to stabilize CIICs in the presence or absence of linker or body disulfide bonds include substitutions at any one or more of 40, 47, 52, and 75 of the α1 domain sequence, or the corresponding locations in the α1 domain sequence of other DPA alleles. Position 47 may be substituted by an aa other than Cys, which in some instances may be a Ser or a positively charged aa such as a Lys (K) or Arg (R) (C47S, C47R or C47K substitutions). Alternatively, position 47 may be Lys, Arg, or Ser, or may be substituted by a Lys, Arg, or Ser, (e.g., a Lys or Ser).

Substitutions at positions 40, 52, and/or 75 of DP α subunit α1 domain sequence may enhance the stability of MHC (HLA) peptide interactions and/or HLA stability. Position 40 may be, or may be substituted by, an acidic residue such as E or D (e.g., an D40E substitution), position 52 may be, or may be substituted by, an H (e.g., a G52H substitution), and position 75 may be, or may be substituted by, an aliphatic aa such as I, L, or V (e.g., a T751, T75L, or T75V) to enhance stability.

### 5. Expression

The CIICs, particularly the duplex soluble CIICs (e.g., having an Ig Fc scaffold such as in FIG. 1, structures A-I and N-X), may be expressed by cells where the CIICs accumulate in the culture media to levels of about 25 mg/I to 350 mg/I of culture media. The cells can be eukaryotic cells, and in particular mammalian cells or cell lines.

Suitable mammalian cell lines include, but are not limited to, HeLa cells (e.g., American Type Culture Collection (ATCC) No. CCL-2™), CHO cells (e.g., ATCC Nos. CRL9618, CCL61, CRL-9618™, CCL-61™, CRL9096), 293 cells (e.g., ATCC No. CRL-1573™), Vero cells, NIH 3T3 cells (e.g., ATCC No. CRL-1658), Huh-7 cells, BHK cells (e.g., ATCC No. CCL10, CCL-10™), PC12 cells (ATCC No. CRL1721, CRL-1721™), COS cells, COS-7 cells (ATCC No. CRL1651), RAT1 cells, mouse L cells (ATCC No. CCLI.3), human embryonic kidney (HEK) cells (ATCC No. CRL1573), and HLHepG2 cells. The cells may express the protein from nucleic acids that are transfected or transduced into the cells, or from nucleic acid sequences that have been stably incorporated into the cells as discussed below under Genetically Modified Host Cells.

The CIICs can be expressed at levels of at least 50 mg/I, and in some instances can reach about 250 mg/I or more, such as about 250 to about 350 mg/l. The CIICs may be expressed in an amount from about 25 mg/I to about 50 mg/I or about 50 mg to about 100 mg/l. The CIICs may be expressed in an amount from about 100 mg/I to about 150 mg/I, or about 150 mg/I to about 200 mg/l. The CIICs may be expressed in an amount from about 200 mg/I to about 250 mg/I, or about 250 mg/I to about 300 mg/l. The CIICs may be expressed in an amount from about 300 mg to about 325 mg/I, or about 325 mg/I to about 350 mg/I (e.g., 330 mg/I). In some cases, expression levels greater than 350 mg/I also may be obtained, e.g., greater than 500 mg/I, greater than 750 mg/I or greater than 1 g/l. Expression can be accomplished, e.g., using CHO cells transfected using the Gibco (Gaithersburg, MD) ExpiCHO® Expression System User Guide Max Titer Protocol. Harvest can be completed on day 12 and expression levels determined. Generally speaking, higher levels of expression can be obtained using stable cell lines as opposed to transient transfection.

### 6. Properties of Stabilized Class II Molecules

The CIICs of the present disclosure are capable of presenting epitopes to TCRs in the context of the CIICs' MHC/HLA sequencs. Where the TCR is specific to the peptide epitope presented by the CIIC it may be functionally engaged leading to TCR signaling. Where the CIIC also comprises a MOD sequence, the MOD may interact with its cognate receptor in the surface of the T cell providing a second signal and directing the T cell response.

The CIICs, duplex CIICs or other higher order CIIC complexes may have one or more properties related to the overall stability (e.g., thermal stability) that are conducive to formulation. The CIICs, particularly when stabilized by a body or linker disulfide as discussed herein, can display favorable stability to freezing and thawing one or more times and can be frozen in saline or phosphate buffered saline (“PBS plus saline,” see Example 4 for the composition) in a −80° C. environment (placement of an aliquot of the protein less than 0.1 ml in volume in a freezer at that temperature) and thawed at room temperature (20° C.) without the need for special handling and equipment such as liquid nitrogen snap-freezing. CIIC stability to freezing and thawing permits repeated freeze-thaw cycles two or more times, or three or more times, without substantial loss of protein due to non-specific aggregation or denaturation. For example, soluble CIICs having an IgG scaffold may be substantially stable to freezing and thawing in PBS plus saline two or more times or three or more times. The samples of protein subject to freeze-thaw testing under those conditions may have less than 10% or less than 7.5% loss of CIIC protein to denaturation or non-specific aggregation based on non-reducing size separation chromatography (e.g., using integration of the peak areas with detection at 280 nm). For some CIIC samples the denaturation after 1, 2 or three rounds of freezing and thawing will be less than 5% or less than 3% (e.g., no detectable loss).

In addition to freeze-thaw stability, the CIICs may display stability to temperatures at or above 37° C. in short term testing and in extended stability testing. CIICs, particularly when stabilized by a body or linker disulfide as discussed herein, can display substantial resistance to thermal denaturing when heated to 40° C. or 42° C. in PBS plus saline (see, e.g., Example 4) for 24 hours or more (one day or more). Accelerated stability testing over 10 days can result in less than 20% or less than 15% of the protein being lost to denaturation or aggregation at 42° C. at 10 days. For some CIICs less than 10% or less than 5% of the protein is lost to thermal denaturation at 42° C. at 10 days. The amount of protein lost to thermal denaturation may be determined based on non-reducing size separation chromatography (e.g., using integration of the peak areas with detection at 280 nm).

Unless stated otherwise, the amount of protein lost to denaturation/aggregation in stability tests (e.g., freeze-thaw and/or exposure to elevated temperatures for accelerated stability testing at 40° C. or 42° C.) is determined by HPLC chromatographic analysis using a Superdex©200 3.2 mm×300 mm column (Pharmacia) to observe the amount (e.g., percentage) of unaggreagated protein present (e.g., molecules of duplex constructs) and protein loss. The column was eluted with PBS plus 500 mM NaCl pH=7.4 and 0.02% sodium azide (see PBS plus saline in Example 4) at a flow rate of 0.15 mL/min at 22° C. Data analyses for chromatography tests were performed using ChemStation® software, with the percent unaggregated protein calculated by integrating the absorbance at 280 nm for the peak corresponding to the molecular weight of the unaggregated protein construct (e.g., duplex CIIC molecules).

Based upon the temperature at which aggregation begins, CIICs may display resistance to denaturation at temperatures greater than about 42° C. or greater than about 45° C. (e.g., the initial temperature at which aggregation begins (Tagg) is from about 42 to about 45° C. For some CIICs the temperature at which aggregation initiates is from about 45 to about 50° C. (e.g., 45.1 to 49.6° C.). CIICs, and particularly duplex CIICs having an Ig Fc scaffold, may display resistance to aggregation at temperatures in excess of 42° C. in PBS plus saline as assessed using size based chromatographic analysis. In some cases, the temperature at which aggregation of the CIIC or higher order CIIC complex (e.g., duplex CIIC) begins (initiates) is from about 42 to about 45° C., or from about 45 to about 50° C. (e.g., 45.1 to 49.6° C.). In some cases, the temperature at which aggregation of the CIIC or higher order CIIC complex (e.g., duplex CIIC) begins (initiates) is from about 50 to about 55° C., or is from about 55 to about 60° C. In some cases, the temperature at which aggregation of the CIIC or higher order CIIC complex (e.g., duplex CIIC) begins (initiates) is from about 60 to about 65° C., or is from about 65 to about 70° C. (e.g., 68.5° C.). The Tagg is determined using the change in optical transmission/absorbance caused by scattering due to aggregation of the protein sample measured using a Nano Temper Prometheus@instrument (NanoTemper Technologies GmbH, Flöβergasse 4, 81369 München, Germany; see, e.g., Example 2). The Tagg is the temperature at which the second derivative of transmission vs temperature line first has a significant inflection as determined by the manufacturer's software. Unless stated otherwise, all measurements are made using 10 mg of protein/ml in PBS plus saline pH 7.4 saline (see Example 4 for composition) from 20° C. to 95° C., with a scan rate of 60° C./hr.

The melting point of CIIC MHC Class II sequences as determined by differential scanning calorimetry (e.g., using a Malvern Panalytical MicroCal DSC) may be greater than about 40° C. or greater than about 45° C. The melting point of the IgG scaffold sequence present as a duplex sequence as determined by differential scanning calorimetry (e.g., using a Malvern Panalytical MicroCal DSC and protein at 1 mg/ml) may be greater than about 70° C. or greater than about 80° C. DSC measurements are made in PBS plus saline pH 7.4 unless indicated otherwise (see, e.g, Example 2).

### D. Nucleic Acids

The present disclosure provides a nucleic acid comprising a nucleotide sequence encoding one or more polypeptides of a CIIC or higher order CIIC complex. Where two or more nucleotide sequences encode two or more polypeptides of a CIIC or higher order CIIC complex, e.g., in cases where interspecific binding sequences are present, then the present disclosure provides a plurality of nucleic acid sequences that collectively encode the polypeptides of a CIIC or higher order CIIC complex. In some cases, the nucleic acid is present in a recombinant expression vector, thus, the present disclosure provides a recombinant expression vector comprising a nucleotide sequence encoding a CIIC, or a plurality of recombinant expression vectors that collectively comprise nucleotide sequences encoding two or more polypeptides of a CIIC or higher order CIIC complex.

### 1. Nucleic Acids Encoding a CIIC or CIIC Forming a Higher Order Complex, Such as a Duplex CIIC

The present disclosure provides nucleic acids comprising a nucleotide sequence encoding a CIIC having a scaffold polypeptide that comprises at least one interspecific or non-interspecific binding sequence or at least one multimerization sequence that permits two CIIC molecules to form dimers or higher order complexes. It will be apparent that individual polypeptides of a CIIC interspecific duplex or multimer may be encoded on a single nucleic acid (e.g., under the control of separate promoters), or alternatively may be located on two or more separate nucleic acids (e.g., plasmids).

### 2. Recombinant Expression Vectors

The present disclosure provides recombinant expression vectors comprising nucleic acids encoding one or more polypeptides of a CIIC or its higher order complexes. In some cases, the recombinant expression vector is a non-viral vector. In some cases, the recombinant expression vector is a viral construct, such as a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors include, but are not limited to, viral vectors (e.g., viral vectors based on vaccinia virus, poliovirus, adenovirus (see, e.g., Li et al., Invest Opthalmol Vis Sci 35:2543 2549, 1994, Borras et al., Gene Ther 6:515 524, 1999, Li and Davidson, PNAS 92:7700 7704, 1995, Sakamoto et al., H Gene Ther 5:1088 1097, 1999, WO 94/12649, WO 93/03769, WO 93/19191, WO 94/28938, WO 95/11984 and WO 95/00655), adeno-associated virus (see, e.g., Ali et al., Hum Gene Ther 9:81 86, 1998, Flannery et al., PNAS 94:6916 6921, 1997, Bennett et al., Invest Opthalmol Vis Sci 38:2857 2863, 1997, Jomary et al., Gene Ther 4:683 690, 1997, Rolling et al., Hum Gene Ther 10:641 648, 1999, Ali et al., Hum Mol Genet 5:591 594, 1996, Srivastava in WO 93/09239, Samulski et al., J. Vir. (1989) 63:3822-3828, Mendelson et al., Virol. (1988) 166:154-165, and Flotte et al., PNAS (1993) 90:10613-10617), SV40, herpes simplex virus, human immunodeficiency virus (see, e.g., Miyoshi et al., PNAS 94:10319 23, 1997, Takahashi et al., J Virol. 73:7812 7816, 1999), a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus), and the like. Numerous suitable expression vectors are known to those of skill in the art, and many are commercially available.

Depending on the host/vector system utilized, any of a number of suitable transcription and translation control elements, including constitutive and inducible promoters, transcription enhancer elements, transcription terminators, etc., may be used in the expression vector (see, e.g., Bitter et al. (1987) Methods in Enzymology, 153:516-544).

In some cases, a nucleotide sequence encoding one or more polypeptides of a CIIC is operably linked to a control element, e.g., a transcriptional control element, such as a promoter. The transcriptional control element may be functional in either a eukaryotic cell, e.g., a mammalian cell such as a human, hamster, or mouse cell, or a prokaryotic cell (e.g., bacterial). In some cases, a nucleotide sequence encoding a DNA-targeting RNA and/or a site-directed modifying polypeptide is operably linked to multiple control elements that allow expression of the nucleotide sequence encoding a DNA-targeting RNA and/or a site-directed modifying polypeptide in both prokaryotic and eukaryotic cells.

Non-limiting examples of suitable eukaryotic promoters (promoters functional in a eukaryotic cell) include the cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

### E. Genetically Modified Host Cells

The present disclosure provides a genetically modified host cell, where the host cell is genetically modified with one or more nucleic acid(s) that encode, or encode and express, CIIC proteins or higher order complexes of CIICs (e.g., duplex CIICs).

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2™), CHO cells (e.g., ATCC Nos. CRL9618, CCL61, CRL-9618™, CCL-61™, CRL9096), 293 cells (e.g., ATCC No. CRL-1573™), Vero cells, NIH 3T3 cells (e.g., ATCC No. CRL-1658), Huh-7 cells, BHK cells (e.g., ATCC No. CCL10, CCL-10™), PC12 cells (ATCC No. CRL1721, CRL-1721™), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like. In some cases, the host cell is a mammalian cell that has been genetically modified such that it does not synthesize endogenous MHC Class II heavy chains (MHC-H).

Genetically modified host cells can be used to produce a CIIC and higher order complexes of CIICs. For example, a genetically modified host cell can be used to produce a duplex CIIC. For example, an expression vector(s) comprising nucleotide sequences encoding the CIIC polypeptide(s) is/are introduced into a host cell, generating a genetically modified host cell, which genetically modified host cell produces the polypeptide(s) (e.g., as an excreted soluble protein).

### F. Methods of Producing CIICs

The present disclosure provides methods of producing soluble CIICs (e.g., duplex CIICs). The methods generally involve culturing, in a culture medium, a host cell that is genetically modified with a recombinant expression vector(s) comprising a nucleotide sequence(s) encoding the CIIC (e.g., a genetically modified host cell of the present disclosure), and isolating the CIIC from the genetically modified host cell and/or the culture medium. As noted above, in some cases, the individual polypeptide chains of interspecific CIICs may be encoded in separate nucleic acids (e.g., recombinant expression vectors). In some cases, all polypeptide chains of a CIIC or their higher order complexes are encoded in a single recombinant expression vector.

Isolation of soluble CIICs from the host cell employed for expression (e.g., from a lysate of the expression host cell) and/or the culture medium in which the host cell is cultured, can be carried out using standard methods of protein purification. For example, a lysate of the host cell may be prepared, and the CIIC purified from the lysate using high performance liquid chromatography (HPLC), exclusion chromatography (e.g., size exclusion chromatography), gel electrophoresis, affinity chromatography, or other purification technique. Alternatively, where the CIIC is secreted from the expression host cell into the culture medium, the CIIC can be purified from the culture medium using HPLC, exclusion chromatography, gel electrophoresis, affinity chromatography, or other purification technique. In some cases, the CIIC is purified, e.g., a composition is generated that comprises at least about 80% by weight, at least about 85% by weight, at least about 95% by weight, or at least about 99.5% by weight, of the CIIC in relation to contaminants related to the method of preparation of the product and its purification. The percentages can be based upon total protein.

In some cases, e.g., where the expressed soluble CIIC comprises an affinity tag or affinity domain, the CIIC can be purified using an immobilized binding partner of the affinity tag. For example, where a CIIC comprises an Ig Fc polypeptide, the CIIC can be isolated from genetically modified mammalian host cells and/or from culture medium comprising the CIIC by affinity chromatography, e.g., on a Protein A column, a Protein G column, or the like. An example of a suitable mammalian cell is a CHO cell, e.g., an Expi-CHO-S™ cell (e.g., ThermoFisher Scientific, Catalog #Δ29127). Affinity chromatography, such as affinity chromatography on protein A or G may be followed by a size based chromatographic, such as size exclusion chromatography, or dialysis separation.

Where applicable, the polypeptides of the CIIC comprising suitable scaffolds will spontaneously form disulfide bonds between, for example, scaffold polypeptides, or scaffold sequences. When both scaffold polypeptides include Ig Fc polypeptides with suitable cysteine residues, disulfide bonds will spontaneously form between the respective Ig Fc polypeptides to covalently link the two scaffolds forming a covalently linked duplex CIIC.

CIICs associated with membranes (e.g., those with MAS or a sequence that results in lipid addition) may be purified from cells expressing the proteins by a variety of means. The cells may be lysed and optionally treated with nucleases to remove the majority of soluble proteins and nucleic acids. Lysis may be accomplished by mechanical (e.g., homogenization and/or sonication) treatment, hypotonic treatment, and/or low levels of detergent/surfactant or organic solvents that open the cells but leave the cell membranes substantially intact. Cell membrane preparations may be collected by, for example, centrifugation and after resuspension the membrane associated CIICs may be purified. Depending on the nature of the membrane interaction (e.g., lipid groups, amphipathic helix, or MAS sequence), differing techniques may be used for purification. For example, further addition of detergent can be used to solubilize the membrane proteins, with higher amounts generally required for CIICs with MAS sequences than CIICs with amphipathic helices or lipid groups. Once detergent solubilized, the proteins may be further separated by other means including affinity chromatography separation, density gradient separation, and the like.

### G. Compositions

### 1. Compositions Comprising a CIIC

The present disclosure provides compositions, including pharmaceutical compositions, comprising a CIIC and/or higher order complexes of CIICs (e.g., duplex CIICs). The compositions may comprise soluble CIICs and/or membrane associated CIICs. Where membrane associated CIICs are present in the composition the composition may be in the form of solubilized cell membranes, surfactant/detergent solubilized CIICs, lipid vesicles or micelles, or artificial antigen presenting cells, such as engineered erythroid cells and enucleated cells (e.g., platelets), that may be used to activate or suppress T cells. Pharmaceutical compositions can comprise, in addition to a CIIC (or a nucleic acid encoding a CIIC), one or more known additives such as carriers, excipients, diluents, buffers, salts, surfactants (e.g., non-ionic surfactants), amino acids (e.g., arginine), etc., a variety of which are known in the art and need not be discussed in detail herein. For example, see the ninth edition or latest edition of Sheskey et al., “Handbook of Pharmaceutical Excipients” and/or Remington: The Science and Practice of Pharmacy,” 23rd Ed., or latest edition, Mack Publishing Co.

In some cases, a subject pharmaceutical composition will be suitable for administration to a subject, e.g., will be sterile and/or substantially free of pyrogens. For example, in some embodiments, a subject pharmaceutical composition will be suitable for administration to a human subject, e.g., where the composition is sterile and is substantially free of detectable pyrogens and/or other toxins, or such detectable pyrogens and/or other toxins are below a permissible limit.

The compositions may, for example, be in the form of aqueous or other solutions, powders, granules, tablets, pills, suppositories, capsules, suspensions, sprays, and the like. The composition may be formulated according to the various routes of administration described below.

Where a CIIC or higher order CIIC complex (e.g., duplex CIIC) is administered as an injectable directly into a tissue or in a space between tissues (e.g., subcutaneously, intraperitoneally, intramuscularly, intralymphatically, and/or intravenously), a formulation can be provided as a ready-to-use dosage form, or as a non-aqueous form (e.g., a reconstitutable storage-stable powder) or an aqueous form, such as liquid composed of pharmaceutically acceptable carriers and excipients. CIICs (e.g., soluble CIICs) may also be provided so as to enhance serum half-life of the subject protein following administration. For example, the protein may be provided in a liposome formulation, prepared as a colloid, or prepared using other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

In some cases, a CIIC composition (e.g., a pharmaceutical composition) comprises: a) a CIIC or higher order CIIC complex (e.g., a duplex CIIC), and b) saline (e.g., 0.9% NaCl), and may be buffered to control pH. In some cases, the composition is sterile and/or substantially pyrogen free, or the amount of detectable pyrogens and/or other toxins are below a permissible limit. In some cases, the composition is suitable for administration to a human subject, e.g., where the composition is sterile and is free of detectable pyrogens and/or other toxins, or the amount of detectable pyrogens and/or other toxins are below a permissible limit. Thus, the present disclosure provides a composition comprising: a) a CIIC or higher order CIIC complex (e.g., duplex CIIC), b) saline (e.g., 0.9% NaCl), and optionally containing c) a buffering agent to control pH, where the composition is sterile and is substantially free of detectable pyrogens and/or other toxins, or such detectable pyrogens and/or other toxins are below a permissible limit (e.g., for intravenous administration).

A pharmaceutical composition can be present in a container, e.g., a sterile container, such as a syringe. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

The concentration of a CIIC in a formulation can vary widely. For example, a CIIC or higher order CIIC complex (e.g., duplex CIIC) may be present from less than about 0.1% (usually at least about 2%) to as much as 20% to 50% or more by weight (e.g., from 1% to 10%, 5% to 15%, 10% to 20% by weight, or 20 to 50% by weight). The concentration will usually be selected primarily based on fluid volumes, viscosities, and patient-based factors in accordance with the particular mode of administration selected and the patient's needs.

The present disclosure provides a container comprising a CIIC-containing composition, e.g., a liquid composition. The container can be, e.g., a syringe, an ampoule, and the like. In some cases, the container is sterile.

In some cases, both the container and the composition are sterile.

### 2. Compositions Comprising a Nucleic Acid or a Recombinant Expression Vector

The present disclosure provides compositions (e.g., pharmaceutical compositions) comprising a nucleic acid or a recombinant expression vector that comprises one or more nucleic acid sequences encoding any one or more CIIC polypeptides (e.g., each of the polypeptides of a higher order CIIC complex having interspecific scaffold sequences). As discussed above, a wide variety of pharmaceutically acceptable excipients is known in the art and need not be discussed in detail herein.

A nucleic acid or a recombinant expression vector composition can include one or more nucleic acids or one or more recombinant expression vectors comprising nucleic acid (e.g., DNA or RNA) sequences encoding a CIIC polypeptide or all polypeptides of a CIIC.

A pharmaceutically acceptable formulation may comprise a nucleic acid or recombinant expression vector encoding one or more polypeptides of a CIIC (e.g., in an amount of from about 0.001% to about 90% (w/w)). In some cases, such pharmaceutical compositions will be suitable for administration to a subject, e.g., will be sterile and/or substantially free of pyrogens. For example, in some embodiments, the pharmaceutical composition will be suitable for administration to a human subject, e.g., where the composition is sterile and is substantially free of detectable pyrogens and/or other toxins, or such detectable pyrogens and/or other toxins are below a permissible limit. Pharmaceutical compositions comprising nucleic acids may include any suitable carier for the nucleic acid, e.g., a lipid nanoparticle. Such carriers are well known to those of skill in the art. Alternatively, the nucleic acids can be delivered in the form of a cell (e.g., a B cell or other blood cell) that comprises the nucleic acid, which then can be expressed as a CIIC on the cell surface.

### H. Methods

CIICs and higher order CIIC complexes (e.g., duplex CIICs) are useful for modulating an activity of a T cell. Thus, the present disclosure provides methods of modulating an activity of a target T cell, the methods generally involving contacting a target T cell with a CIIC or a higher order CIIC complex (e.g., a duplex CIIC).

### 1. Methods of Modulating T Cell Activity

The present disclosure provides a method of selectively modulating the activity of an epitope-specific T cell (a target T cell). The method comprises contacting the T cell with a CIIC comprising an epitope recognized by the TCR of the target T cell, where contacting the T cell with a CIIC selectively modulates the activity of the epitope-specific T cell. The present disclosure provides a method of selectively modulating the activity of a T cell that is specific for a T1D- or celiac-epitope, the method comprising contacting the T cell with a CIIC comprising a pMHC that presents a T1D-epitope or celiac-epitope, where contacting the T cell with the CIIC selectively modulates the activity of the T cell specific for the T1D-epitope or celiac-epitope presented by the CIIC. In some cases, the contacting occurs in vivo (e.g., in a mammal such as a human, rat, mouse, dog, cat, pig, horse, or primate). In some cases, the contacting occurs in vitro.

In some cases, a CIIC reduces activity of an autoreactive T cell and/or an autoreactive B cell. In some cases, a CIIC increases the number and/or activity of a regulator T cell (T reg), resulting in reduced activity of an autoreactive T cell and/or an autoreactive B cell.

In some cases, a CIIC is contacted with an epitope-specific CD4+ T cell. In some cases, the epitope-specific T cell is a CD4+CD8+(double positive) T cell (see, e.g., Boher et al Front. Immunol., 29 Mar. 2019 on the world wide web at doi.org/10.3389/fimmu.2019.00622, and Matsuzaki et al. J. Immuno.Therapy of Cancer 7: Article number: 7 (2019)). In some cases, the epitope-specific T cell is an NK-T cell (see, e.g., Nakamura et al. J, Immunol. 2003 Aug. 1, 171(3):1266-71). In some cases, the epitope-specific T cell is a T reg. The contacting may result in modulating the activity of a T cell, which can result in, but is not limited to, proliferation and/or maintenance of regulatory T cells, for example when IL-2 MODs and/or TGF-β MODs (such as masked TGF-β MODs) are present. The effects of IL-2 and/or TGF-β MODs may be modified by the presence of retinoic acids such as all trans retinoic acid.

A CIIC may be contacted with an epitope-specific CD4+ T cell. The CD4+ T cell may be a T helper (Th) type 1 (Th1) cell that produces, among other things, interferon gamma, and which may be a target for inhibition in autoimmune conditions (e.g., in MS). The CD4+ T cell may be a T helper type 2 (Th2) cell that produces, among other things, IL-4. Th2 cells may be inhibited to suppress autoimmune diseases such as asthma and conditions such as allergies. Th2 cells may be inhibited to suppress autoimmune diseases such as T1D or celiac disease. The CD4⁺ T cell may be a T helper type 17 (Th17) cell that produces, among other things, IL-17, and which may be inhibited to suppress autoimmune diseases such as rheumatoid arthritis or psoriasis. Th17 cells specific for a celiac- or T1D-associated peptide epitope may be inhibited to suppress T1D or celiac disease. The CD4⁺ T cell may be a T helper type 9 (Th9) cell that produces, among other things, IL-9, and which may be inhibited to suppress its actions in autoimmune conditions such as multiple sclerosis. The CD4⁺ T cell may be a Th9 cell that produces, among other things, IL-9, and which may be inhibited to suppress its actions in autoimmune conditions such as T1D or celiac disease. The CD4⁺ T cell may be a T follicular helper (Tfh) cell that produces, among other things, IL-21 and IL-4, and which may be inhibited to suppress autoimmune diseases such as asthma and allergies. The CD4⁺ T cell may be a Tfh cell which may be inhibited to suppress autoimmune diseases such as T1D or celiac disease.

In some cases, the T cell being contacted with a CIIC is a regulatory T cell (T reg) that is CD4⁺, FOXP3⁺, and CD25⁺. T regs can suppress autoreactive T cells.

The present disclosure provides a method of increasing proliferation of T regs, the method comprising contacting T regs with a CIIC, where the contacting increases proliferation of T regs specific for an epitope presented by the CIIC. The present disclosure provides a method of increasing proliferation of T regs specific for peptide epitopes of T1D-associated antigens or celiac disease-associated antigens, the method comprising contacting T regs with a CIIC that presents a T1D-epitope or celiac-epitope, where the contacting increases proliferation of T regs specific for epitopes presented by the CIIC. The present disclosure provides a method of increasing the number of epitope specific T regs in an individual, the method comprising administering to the individual a CIIC, where the administering results in an increase in the number of T regs specific to the epitope presented by the CIIC in the individual. The present disclosure provides a method of increasing the number of epitope specific T regs in an individual, the method comprising administering to the individual a CIIC presenting a T1D-epitope or celiac-epitope, where the administering results in an increase in the number of T regs specific to the epitope presented by the CIIC in the individual. For example, the number of T regs specific to the epitope presented by the CIIC can be increased by at least 5%, at least 10%, at least 15%, at least 20%, at least 25%, at least 30%, at least 40%, at least 50%, at least 85%, at least 90%, at least 2-fold, at least 2.5-fold, at least 5-fold, at least 10-fold, or more than 10-fold.

The cell being contacted with a CIIC may be a helper T cell, where contacting the helper T cell with a CIIC inhibits or blocks the proliferation and/or differentiation of Th1 and/or Th2 cells specific/selective for the epitope presented by the CIIC by, for example, inhibiting the expression of the transcription factors T-bet and/or GATA3. The suppression of Th1 and/or Th2 cells results in the decreased activity and/or number of effector cells such as CD8⁺ cytotoxic T cells specific to the epitope.

In some cases a CIIC interacts with T cells that are subject to IL-2 receptor activation provided either by an IL-2 MOD of the CIIC or IL-2 in the T cell environment resulting in: (i) activation, proliferation, or maintenance of T reg cells specific for the epitope presented by the CIIC, (ii) suppression of epitope specific Th1 cell development, (iii) suppression of epitope specific Th2 cell development, and/or (iv) suppression of epitope specific cytotoxic T lymphocyte (CTL) development. The addition of retinoic acid (e.g., all trans retinoic acid) may potentiate the action of the TGF-β-bearing CIICs described herein in any of those functions, particularly activation, proliferation, or maintenance of T reg cells where the CIIC bears one or more IL-2 MODs. Where the epitope is an epitope of an autoantigen, the CIIC can be utilized to suppress an autoimmune response to the epitope and effect a treatment of the autoimmune disease. Where the epitope is an allergen the CIIC can be utilized to suppress allergic responses to the epitope. Where the epitope is part of an antigen presented by a tissue graft, the CIIC can be utilized to suppress HVGD. Where the epitope is part of a host antigen recognized by a grafted tissue, the CIIC can be utilized to suppress GVHD. Where the epitope is a T1D-epitope the CIIC can be utilized to suppress responses to the epitope and effect treatment of T1D. Where the epitope is a celiac-epitope, the CIIC can be utilized to suppress responses to the epitope and effect treatment of celiac disease.

CIICs may interact with T cells in the presence of IL-2 and PD1 receptor agonist, either or both of which may be provided by IL-2 or PD-L1 MODs of the CIIC and/or IL-2 or PD-L1 present in the T cell's environment during the interaction. Under such conditions the CIIC along with agonist of the IL-2 and PD1 receptors may regulate the development, maintenance, and function of T reg cells (e.g., induced regulatory T cells) specific for the epitope presented by the CIIC. PD-L1 also synergizes with TGF-β to promote iT reg cell development. See, e.g., Francisco et al., J Exp Med., 206(13):3015-3029 (2009). Accordingly, masked TGF-β MOD-bearing CIICs along with agonist of the IL-2 receptor and PD1 receptor (e.g., a CIIC bearing one or more masked TGF-β MODs and additionally one or more IL-2 MODs and/or one or more PD-L1 MODs) may be employed to suppress immune responses to, for example, epitopes of autoantigens, allergens, antigens presented by grafted tissues (HVGD), and autoantigens in GVHD. In further methods, masked TGF-β MOD-bearing CIICs along with agonist of the IL-2 receptor and PD1 receptor (e.g., a CIIC bearing one or more masked TGF-β MODs and additionally one or more IL-2 MODs and/or one or more PD-L1 MODs) may be employed to suppress immune responses to epitopes of autoantigens associated with, for example, T1D or celiac disease.

### 2. Methods of Detecting an Antigen-Specific T Cell

The present disclosure provides methods of detecting an antigen-specific T-cell. The methods comprise contacting a T cell with a CIIC either lacking a MOD or bearing a MOD with minimal affinity for its co-MOD, and detecting binding of the CIIC to the T cell. Binding of such a CIIC to the T cell indicates that the T cell is specific for the epitope present in the CIIC. In some cases, the CIIC comprises a detectable label. Suitable detectable labels include, but are not limited to, a radioisotope, a fluorescent polypeptide, or an enzyme that generates a fluorescent product, and an enzyme that generates a colored product. Where the CIIC comprises a detectable label, binding of the CIIC to the T cell is detected by detecting the detectable label.

In some cases, a CIIC comprises a detectable label suitable for use in in vivo imaging, e.g., suitable for use in positron emission tomography (PET), single photon emission tomography (SPECT), near infrared (NIR) optical imaging, x-ray imaging, computer-assisted tomography (CAT), or magnetic resonance imaging (MRI), or other in vivo imaging method. Examples of suitable labels for in vivo imaging include gadolinium chelates (e.g., gadolinium chelates with DTPA (diethylenetriamine penta-acetic acid), DTPA-bismethylamide (BMA), DOTA (dodecane tetraacetic acid), or HP-DO3A (1,4,7-tris(carboxymethyl)-10-(2′-hydroxypropyl)-1,4,7,10-tetraazacyclododecane)), iron chelates, magnesium chelates, manganese chelates, copper chelates, chromium chelates, iodine-based materials, and radionuclides.

Suitable fluorescent proteins for detecting T cells using CIICs include, but are not limited to, green fluorescent protein (GFP) or variants thereof, blue fluorescent variants of GFP (BFP), cyan fluorescent variants of GFP (CFP), yellow fluorescent variants of GFP (YFP), enhanced GFP (EGFP), enhanced CFP (ECFP), enhanced YFP (EYFP), and the like. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes that may be employed as labels include, but are not limited to, horseradish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

In some cases, binding of the CIIC to the T cell is detected using a detectably labeled antibody specific for the CIIC. An antibody specific for the CIIC can comprise a detectable label such as a radioisotope, a fluorescent polypeptide, an enzyme that generates a fluorescent product, or an enzyme that generates a colored product.

In some cases, the T cell being detected is present in a sample comprising a plurality of T cells. For example, a T cell being detected can be present in a sample comprising from 10 to 109 T cells, e.g., from 10 to 10², from 10² to 10⁴, from 10⁴ to 10⁶, from 10⁶ to 10⁷, from 10⁷ to 10⁸, or from 10⁸ to 10⁹, or more than 10⁹ T cells.

### 3. Treatment Methods

The present disclosure provides treatment methods comprising administering to an individual an amount of a CIIC (e.g., a duplex CIIC), or one or more nucleic acids or expression vectors encoding one or more CIICs effective to selectively modulate the activity of an epitope-specific T cell in an individual, and to treat the individual. The present disclosure further provides treatment methods comprising administering to an individual a composition comprising an amount of a CIIC or higher order CIIC complex (e.g., a duplex CIIC) effective to selectively modulate the activity of a T1D-epitope-specific T cell or a celiac-epitope specific T cell in the individual, and to treat the individual. In some cases, a treatment method comprises administering to an individual in need thereof a pharmaceutical composition comprising an effective amount of a CIIC (e.g., a duplex CIIC) useful for treating T1D occurring in human patients and in experimental animal models (e.g., the non-obese diabetic (NOD) mouse and the Biobreeding (BB) rat). In some cases, a treatment method comprises administering to an individual in need thereof one or more recombinant expression vectors comprising nucleotide sequences encoding one or more CIICs (e.g., a CIIC that may assemble into a duplex or higher order CIIC complex). CIICs expressed by one or more nucleic acids or expression vectors may assemble into one or more higher order complexes (e.g., duplexes) that modulate the activity of an epitope-specific T cell in an individual and treat the individual. In some cases, a treatment method comprises administering to an individual in need thereof one or more mRNA molecules comprising nucleotide sequences encoding a CIIC. The conditions that can be treated include cancers, allergies, infections by viral and non-viral agents, GVHD, HVGD, metabolic disorders, and/or autoimmune disorders including, but not limited to, T1D and/or celiac disease.

The present disclosure provides a method of selectively modulating the activity of an epitope-specific T cell in an individual comprising administering to the individual an effective amount of a CIIC, or one or more nucleic acids (e.g., expression vectors, mRNA, etc.) comprising nucleotide sequences encoding a CIIC, that may assemble into a higher order complex that selectively modulates the activity of the epitope-specific T cell in the individual. The present disclosure provides a method of selectively modulating the activity of either a T1D-epitope-specific or celiac-epitope-specific T cell in an individual comprising: administering to the individual a pharmaceutical composition comprising an effective amount of a CIIC presenting a T1D-epitope or celiac-epitope, or higher order CIIC (e.g., a duplex CIIC) thereof, where the CIIC selectively modulates the activity of the T1D-epitope-specific or celiac-epitope-specific T cell in the individual. Selectively modulating the activity of an epitope-specific T cell can treat a disease or disorder, including but not limited to cancers, allergies, infections by viral and non-viral agents, GVHD, HVGD, metabolic disorders and/or autoimmune disorders, including T1D and/or celiac disease, in an individual. Thus, the present disclosure provides a treatment method comprising administering to an individual in need thereof a pharmaceutical composition comprising an effective amount of a CIIC sufficient to effect treatment of a disease or disorder including T1D or celiac disease.

In some cases, a CIIC may comprise in addition to a masked TGF-β MOD at least one or at least two wt. and/or variant IL-2 MOD polypeptide sequence(s). Where the epitope of the CIIC is an epitope of an autoantigen (self-epitope), the CIIC may selectively activate, cause the proliferation of, and/or support the survival of a T reg cell specific for the epitope, and administration of the CIIC may be used to treat an autoimmune disease involving an immune response to the autoantigen. Where the epitope presented by the CIIC is T1D-epitope or celiac-epitope, the CIIC (e.g., a CIIC complex such as a duplex CIIC) may be utilized for treating T1D or celiac disease.

In some cases, CIICs may comprise in addition to a masked TGF-β MOD at least one or at least two wt. and/or variant PD-L1 MOD sequence(s). Where the epitope of the CIIC is an epitope of an autoantigen such CIICs may selectively activate, cause the proliferation of, and/or support the survival of a T reg cell specific for the epitope presented by the CIIC. See, e.g., Stathoupoulou et al., Immunity, 49(2): 247-263 (2018). Accordingly, such CIICs may be used to suppress an immune response (e.g., an autoimmune, GVHD, or HVGD response) to the epitope and/or to treat a disease associated with an immune response to the epitope.

In some cases, a CIIC may comprise in addition to a masked TGF-β MOD at least one or at least two wt. and/or variant PD-L1 MOD sequence(s), and in addition, at least one or at least two wt. and/or variant IL-2 MOD sequence(s). Where the peptide epitope of the CIIC is a peptide epitope of an autoantigen, the CIIC selectively activates, causes the proliferation of, and/or supports the survival of a T reg cell specific for the epitope presented by the CIIC. Accordingly, such CIICs may be used to suppress an immune response (e.g., an autoimmune, GVHD, or HVGD response) to the epitope and/or to treat a disease associated with an immune response to the epitope. Sufficient IL-2 may be present in the environment where contacting occurs such that the presence of an IL-2 MOD is not required.

A CIIC may comprise in addition to a masked TGF-β MOD at least one or at least two wt. or variant 4-1BBL MOD sequence(s). A CIIC may also comprise at least one wt. or variant 4-1BBL MOD sequence, and in addition, at least one wt. and/or variant IL-2 MOD sequence(s). CIICs comprising at least one 4-1BBL MOD, or at least one 4-1BBL MOD alone or in combination with at least one wt. or variant IL-2 MOD, can selectively activate, cause the proliferation of, and/or support the survival of T reg cells specific for the epitope presented by the CIIC. See, e.g., Elpek et al. J Immunol, 179:7295-7304 (2020) discussing the effect of IL-2 and 4-1BB signaling on T reg expansion. Accordingly, such CIICs may be used to suppress an immune response (e.g., an autoimmune, GVHD, or HVGD response) to the epitope and/or to treat a disease associated with an immune response to the epitope. Sufficient IL-2 may be present in the environment where contacting occurs such that the presence of an IL-2 MOD is not required.

The present disclosure provides a method of treating an autoimmune disorder in an individual comprising administering to the individual an effective amount of a CIIC (e.g., a duplex CIIC), or one or more nucleic acids comprising nucleotide sequences encoding one or more CIICs (which may assemble into a higher order complex such as a duplex CIIC), where the CIIC comprises an epitope of an autoantigen. In some cases an “effective amount” of a CIIC is an amount that, when administered in one or more doses to an individual in need thereof reduces the number of self-reactive CD4⁺ cells that have a TCR that recognizes (is specific for) the epitope presented by the CIIC by, for example, at least 10%, at least 15%, at least 20%, at least 25%, at least 30%, at least 40%, at least 50%, at least 60%, at least 70%, at least 80%, at least 90%, or at least 95% (e.g., from 10% to 50%, or from 50% to 95%) compared to the number of self-reactive T cells in the individual before administration of the CIIC, or in the absence of administration of the CIIC. An “effective amount” of a CIIC may be an amount that, when administered in one or more doses to an individual in need thereof, reduces production of one or more Th2 cytokines (e.g., IL-4, IL-5, and/or IL-13) in the individual or a tissue of an individual. An “effective amount” of a CIIC or higher order CIIC complex may be an amount that, when administered in one or more doses to an individual in need thereof, ameliorates one or more symptoms associated with an autoimmune disease in the individual. Administration of a CIIC (or higher order CIIC complex) may reduce the number or activity of CD4*self-reactive T cells specific for the epitope of the CIIC, which may in turn lead to a reduction in CD8⁺ self-reactive T cells. Administration of a CIIC (or higher order CIIC complex) may increase the number of CD4⁺ T regs, which in turn reduces the number of CD4⁺ self-reactive T cells and/or self-reactive CD8⁺ T cells present in an individual prior to administration of the CIIC.

The present disclosure further provides a method of treating an autoimmune disease (e.g., T1D or celiac disease) in an individual, the method comprising administering to the individual a pharmaceutical composition comprising an effective amount of a CIIC or higher order CIIC complex (e.g., a duplex CIIC), where the CIIC comprises an epitope of an autoantigen (e.g., a T1D-epitope or celiac-epitope as described above), and where the CIIC comprises PD-L1. In some cases, an “effective amount” of a CIIC or higher order CIIC is an amount that, when administered in one or more doses to an individual in need thereof, reduces the number of self-reactive CD4⁺ T cells that react with the epitope of the CIIC by, for example, at least 10%, at least 15%, at least 20%, at least 25%, at least 30%, at least 40%, at least 50%, at least 60%, at least 70%, at least 80%, at least 90%, or at least 95% (e.g., from 10% to 50%, or from 50% to 95%) compared to the number of those self-reactive CD4⁺ T cells in the individual before administration of the CIIC, or in the absence of administration with the CIIC. In some cases, an “effective amount” of a CIIC is an amount that, when administered in one or more doses to an individual in need thereof, reduces production of Th1 cytokines (e.g., IL-2, IL-10, and TNF-alpha/beta) in the individual or a tissue of the individual (e.g., by 10%-50% or by greater than 50%).

As noted above, in carrying out a subject treatment method, a CIIC is administered to an individual in need thereof, as the polypeptide per se. In other instances, in carrying out a subject treatment method, one or more nucleic acids comprising nucleotide sequences encoding a CIIC is/are administered to an individual in need thereof. In other instances, one or more recombinant expression vectors comprising one or more nucleic acid sequences encoding a CIIC of the present disclosure is/are administered to an individual in need thereof.

A CIIC, or one or more nucleic acids encoding such molecules, may be administered alone or with one or more additional therapeutic agents or drugs. The therapeutic agents may be administered before, during, or subsequent to administration of CIIC or higher order CIIC complex or nucleic acids encoding such molecules. When the additional therapeutic agents are administered with a composition or formulation comprising a CIIC or nucleic acids encoding such molecules, the therapeutic agent may be administered concurrently with the CIIC. Alternatively, the therapeutic agents may be co-administered with the CIIC as part of a formulation or composition comprising the CIIC.

Suitable therapeutic agents or drugs that may be administered with or provided as a payload of a CIIC include virtually any therapeutic agent, including small molecule therapeutics (e.g., less than 2,000 Daltons in molecular weight) approved by the U.S. Food and Drug Administration, listed in the 2020 U.S. Pharmacopeia or National Formulary. In embodiments, those therapeutic agents or drugs are less than 1,000 or 2,000 molecular weight. Suitable drugs include antibiotics and various immunosuppressive agents.

When suppression of an immune response (e.g., an autoimmune, GVHD, or HVGD response) is desired, suitable therapeutic agents that may be administered with a CIIC include glucocorticoids. Glucocorticoids are both anti-inflammatory and immunosuppressive and, accordingly, may be useful when CIICs are utilized for the treatment of, for example, autoimmune disease, GVHD, HVGD, metabolic disorders, or allergic reactions. Inhibitors of the mammalian target of rapamycin or “mTOR,” including rapamycin (sirolimus) itself and its analogs (e.g., temsirolimus, everolimus, ridaforolimus, umirolimus, and zotarolimus), may also be administered with, or attached to (e.g., as a payload), a CIIC. mTOR inhibitors such as rapamycin inhibit cytokine-driven proliferation of lymphocytes and activation of T effector and B cells by, for example, reducing their sensitivity to IL-2. See, e.g., Mukherjee et al., J. Transplant, 2009, Article ID 701464, 20 pages doi:10.1155/2009/701464. mTOR inhibitors may be administered with, or attached to, a CIIC that comprises, in addition to its masked TGF-β MOD, optionally at least one, or at least two, wt. and/or variant IL-2 MOD(s).

Amphiregulin, which has been linked to the ability of T regs to suppress autoimmune diseases, may be administered with a CIIC (e.g., containing one or more IL-2, 4-1BBL, and/or PD-L1 MODs) or higher order CIIC complexes thereof. See, e.g., MacDonald et. al., Front Pharmacol, 8: 575 (2017).

Other suitable therapeutic agents that may be administered with a CIIC (e.g., a higher order CIIC complex) comprise one or more agents or antibodies directed against: B lymphocyte antigens (e.g., ibritumomab tiuxetan, obinutuzumab, ofatumumab, rituximab to CD20, brentuximab vedotin directed against CD30, and alemtuzumab to CD52), agents that bind to CD80 and/or CD86 receptors and inhibit T cell proliferation and/or B cell immune response (e.g., abatacept), PD-1 (e.g., nivolumab and pembrolizumab targeting a check point inhibition), RANKL (e.g., denosumab), CTLA-4 (e.g., ipilimumab targeting check point inhibition), agents that bind to the IL-1 receptor competitively with IL-1 (e.g., anakinra), IL-6 (e.g., siltuximab), disialoganglioside (GD2), (e.g., dinutuximab) disialoganglioside (GD2), CD38 (e.g., daratumumab), SLAMF7 (Elotuzumab), both EpCAM and CD3 (e.g., catumaxomab), both CD19 and CD3 (blinatumomab), or agents that block one or more actions of tumor necrosis factor alpha (e.g., an anti-TNF alpha such as golimumab, infliximab, certolizumab, adalimumab or a TNF alpha decoy receptor such as etanercept). Such antibodies would, as a generality, not be administered in conjunction with a CIIC (e.g., a duplexed CIIC) that comprises a sequence to which any of the administered antibodies bind, or under circumstances where the antibody may block the action of a MOD present in the administered CIIC.

The present disclosure provides treatment methods comprising administering to an individual (e.g., an individual in need thereof) an amount of a CIIC, or an amount of one or more nucleic acids or expression vectors encoding the CIIC, effective to selectively modulate the activity of an epitope-specific T cell in the individual and to treat the individual. A treatment method may comprise administering to an individual in need thereof one or more recombinant expression vectors comprising nucleotide sequences encoding a CIIC. A treatment method may comprise administering to an individual in need thereof one or more mRNA molecules comprising nucleotide sequences encoding a CIIC.

As discussed above, the present disclosure provides a method of selectively modulating the activity of an epitope-specific T cell (e.g., a T reg) in an individual, the method comprising administering to the individual an effective amount of a CIIC, or one or more nucleic acids (e.g., expression vectors, mRNA, etc.) comprising nucleotide sequences encoding the CIIC, which selectively modulates the activity of the epitope-specific T cell (e.g., a T reg) in the individual. Selectively modulating the activity of an epitope-specific T cell (e.g., a T reg) can treat a disease or disorder in the individual. Thus, the present disclosure provides a treatment method comprising administering to an individual in need thereof an effective amount of a CIIC or higher order CIIC complex in order to treat a disease or disorder (e.g., a cancer, an autoimmune disease, GVHD, HVGD, an allergy, or an infection).

In some cases, an “effective amount” of a CIIC is an amount that, when administered in one or more doses to an individual in need thereof, reduces production of Th17 cytokines (e.g., IL-17A, IL-17F, and/or IL-22) in the individual or a tissue of the individual. In some cases, an “effective amount” of a CIIC is an amount that, when administered in one or more doses to an individual in need thereof, ameliorates one or more symptoms associated with T1D or celiac disease in the individual.

Administration of a CIIC presenting a celiac-epitope or T1D-epitope may reduce the number of CD4⁺ self-reactive T cells (i.e., the number of CD4⁺ T cells reactive with the T1D-epitope or celiac-epitope), which in turn may lead to a reduction in CD8⁺ self-reactive T cells. In some instances, the CIIC increases the number or activity (e.g., IL-10 and/or TGF-β production) of CD4⁺ T regs specific for the epitope (e.g., a T1D-epitope or celiac-epitope) presented by the CIIC, which in turn may reduce the number or activity of CD4⁺ self-reactive T cells, B cells, and/or self-reactive CD8⁺ T cells specific for that epitope.

The present disclosure provides a method of reducing elevated blood sugar (e.g., glucose) in an individual (e.g., a mammal such as a human) having or suspected of having T1D, the method comprising administering to the individual an effective amount of a CIIC, or one or more nucleic acids comprising nucleotide sequences encoding the CIIC, where the CIIC comprises a T1D-epitope (as described above). The individual may be a human having a fasting blood sugar in excess of 130 or about 140 mg/dL, or postprandial blood sugar in excess of 180 or about 200 mg/dL, and the treatment reduces fasting blood sugar (e.g., such as to a level below 130 mg/dL) or post-prandial blood sugar (e.g., to less than 180 mg/dL) in the individual relative to the level prior to receiving the CIIC. The reduction in blood sugar may be maintained for a period of at least about a week, at least about two weeks, at least about a month (30 days), or more than one month.

The present disclosure provides a method of reducing prediabetic glycosylated hemoglobin referred to as hemoglobin Δ1C (also referred to as hemoglobin Δ1c or HbAlc) levels (in the range of 5.7% to 6.4%) or diabetic hemoglobin Δ1C levels (above 6.4%) in an individual (e.g., a mammal such as a human) having or suspected of having T1D, the method comprising administering to the individual an effective amount of a CIIC, or one or more nucleic acids comprising nucleotide sequences encoding the CIIC, where the CIIC comprises a T1D-epitope (as described above). The treatment reduces diabetic hemoglobin Δ1C (e.g., to less than 6.4% and preferably to less than 5.7%), or prediabetic Δ1C (e.g., such as to less than 5.7% and into the normal range) in the individual relative to the level prior to receiving the CIIC. The reduction in hemoglobin Δ1C may be maintained for a period of at least about a week, at least about two weeks, at least about a month (30 days), or more than one month.

The present disclosure provides treatment methods comprising administering to an individual a composition comprising an amount of a CIIC presenting a celiac-epitope or higher order CIIC (e.g., a duplex CIIC) thereof, effective to selectively modulate the activity of a celiac-epitope-specific T cell in an individual and to treat the individual. In some cases, a treatment method comprises administering to an individual in need thereof a pharmaceutical composition comprising an effective amount of a CIIC comprising a celiac-epitope or higher order CIIC complex (e.g., a duplex CIIC) useful for treating celiac disease occurring in human patients and in experimental animal models.

The present disclosure also provides treatment methods comprising administering to an individual a composition comprising an effective amount of a CIIC (e.g., a duplex CIIC) bearing an immunoglobulin sequence that can support complement-dependent cytotoxicity (CDC), antibody-dependent cell cytotoxicity (ADCC), and/or antibody-dependent cellular phagocytosis (ADCP). Such CIICs selectively engage with T cells specific for the epitope presented by the CIIC in the individual, and treat the individual by depleting T cells specific for the epitope presented by the CIIC by CDC, ADCP, and/or ADCC. CIICs used to promote CDC, ADCP, and/or ADCC may have no MOD sequence or a MOD sequence that has limited affinity for its co-MOD, provided the CIIC is specific for the target T cell. For example, an individual having T1D and expressing a T1D-epitope is administered a CIIC that presents a TID-epitope and is capable of inducing ADCC, ADCP, and/or CDC. The administration results in depletion of T cells specific for the T1D-epitope presented by the CIIC in the individual, thereby treating the T1D disease of the individual. Substitutions in Ig Fc sequences that can enhance CDC, ADCP, and/or ADCC are described in, for example, Wang et al. Protein Cell. 9(1): 63-73 (2018). A nucleic acid or vector comprising a nucleic acid sequence encoding the CIIC may be administered in place of the CIIC protein.

CIICs may also be used to treat cancers resulting from the proliferation of white blood cells including, but not limited to, CD4⁺ T cell lymphomas or leukemias with TCRs specific for the epitope presented by the CIIC. Administering a CIIC presenting an epitope recognized by a CD4* cancer cell may inhibit the proliferation of such T cells through the action of inhibitory MODs such as FAS-L. MODs such as FAS-L when present in a CIIC may reduce the proliferation of T cells specific for the epitope presented by the T cell, or result in their apoptosis or activation-induced cell death (ACID). For example, such a CIIC may cause a reduction in the proliferation and/or activity of the cancerous CD4⁺ T cells and/or T regs (e.g., FoxP3*, CD4⁺ T reg cells) specific for the epitope presented by the CIIC. Alternatively, or in addition to the results of the CIIC's MOD(s) interacting with the CD4*T cells specific for the epitope presented by the CIIC, the cells may be subject to CDC, ADCP, and/or ADCC where the CIIC bears an Ig Fc polypeptide. As above, ADCC, CDC and ADCP properties can be enhanced through substitutions in Ig Fc sequences present in CIICs.

As Fc receptor engagement can be both activating and inhibitory depending on the Fc receptor engaged, MODs that bypass the need for Fc receptor engagement (specifically the inhibitory MOD receptors) may also be incorporated into CIICs for antigen specific CD4⁺ T cell ablation. An exemplary MOD for such purpose would be an anti-CD16a fragment (e.g., a scFv, nano body, or other binding fragment).

As noted above, in carrying out a subject treatment method a CIIC may be administered to an individual in need thereof, as the polypeptide per se. In other instances, in carrying out a subject treatment method, one or more nucleic acids comprising nucleotide sequences encoding a CIIC is/are administered to an individual in need thereof. Accordingly, one or more recombinant expression vectors of the present disclosure may be administered to an individual in need thereof.

### 4. Methods of Selectively Delivering a MOD

The present disclosure provides methods of delivering one or more MODs such as wt. or variant TGF-β (e.g., masked TGF-β), IL-2, IL-10, 4-1BBL, or PD-L1 (e.g., an IL-2 variant disclosed herein) to a selected T cell or a selected T cell population, e.g., in a manner such that a T cell bearing a TCR specific for the epitope presented by a CIIC is targeted. As used herein, the phrases “selectively delivers” and “selectively provides” mean that the majority of T cells for which the CIIC provides detectable modulation comprise a TCR that specifically or preferentially binds the epitope of the CIIC.

The present disclosure thus provides a method of delivering a MOD such as a PD-L1 polypeptide, or a reduced-affinity variant of a MOD such as a PD-L1 variant, selectively to a target T cell bearing a TCR specific for the peptide epitope sequence presented by a CIIC. The present disclosure provides a method of delivering a TGF-β and/or an IL-2 MOD sequence (or a reduced-affinity variant of IL-2) selectively to a target T cell bearing a TCR specific for the peptide epitope presented by a CIIC.

The method of selectively delivering a MOD comprises contacting a population of T cells with a CIIC bearing one or more of the MODs to be selectively delivered. The population of T cells can be a mixed population that comprises: i) the target T cell with a TCR specific to a target epitope, and ii) non-target T cells that are not specific for the target epitope presented by the CIIC-associated peptide epitope (e.g., T cells that are specific for an epitope(s) other than the epitope to which the epitope-specific T cell binds). Epitope-specific T cells that are specific for the peptide epitope present in the CIIC (e.g., duplex CIIC) bind to the pMHC complex provided by the CIIC thereby delivering the MOD of the CIIC (e.g., PD-L1, IL-2, or a reduced-affinity variant thereof) selectively to the bound T cells. Thus, the present disclosure provides a method of delivering TGF-β (e.g., as a masked TGF-β construct or complex), an IL-2 MOD, PD-L1 MOD, and/or a reduced-affinity variant of IL-2 and/or PD-L1, selectively to T cells selective for the epitope presented by the CIIC. Similarly, the disclosure provides a method of delivering TGF-β and/or an IL-2 MOD (or a reduced-affinity variant of an IL-2 MOD) to a target T cell that is selective for the epitope presented by the CIIC. In some cases, the IL-2 MOD bears a substitution at position H16 and/or F42 (e.g., H16A and F42A substitutions) (see supra SEQ ID NO:209). For example, a CIIC or higher order CIIC complex may be contacted with a population of T cells comprising: i) target T cells that are specific for the epitope present in the CIIC or higher order CIIC complex, and ii) non-target T cells, e.g., T cells that are specific for a second epitope(s) that is not the epitope present in the CIIC or higher order CIIC complex. When a CIIC is contacted with such a population, the contacting results in substantially selective delivery of the MOD(s) present in the CIIC (e.g., variant MODs) to the target T cell.

The population of T cells to which a MOD and/or variant MOD is selectively delivered may be in vivo. The population of T cells to which a MOD and/or variant MOD is selectively delivered may be in vitro. For example, a mixed population of T cells may be obtained from an individual and contacted with a CIIC in vitro. Such contacting, which can comprise single or multiple exposures of the T cells to one or more defined doses and/or exposure schedules in the context of in vitro cell culture, can be used to determine whether the mixed population of T cells includes T cells that are specific for the epitope presented by the CIIC. The presence of T cells that are specific for the epitope presented by the CIIC can be determined by assaying a sample comprising a mixed population of T cells, which population of T cells comprises T cells that are not specific for the epitope (non-target T cells) and may comprise T cells that are specific for the epitope (target T cells). Known assays can be used to detect the desired modulation of the target T cells, thereby providing an in vitro assay that can determine whether a particular CIIC possesses an epitope that binds to T cells present in the individual, and thus whether the CIIC has potential use as a therapeutic composition for that individual. Suitable known assays for detection of the desired modulation (e.g., activation/proliferation or inhibition/suppression) of target T cells include, e.g., flow cytometry characterization of T cell phenotype, numbers, and/or antigen specificity. Such an assay to detect the presence of epitope-specific T cells, e.g., a companion diagnostic, can further include additional assays (e.g., effector cytokine ELISpot assays) and/or appropriate controls (e.g., antigen-specific and antigen-nonspecific multimeric peptide-HLA staining reagents) to determine whether the CIIC or higher order CIIC complex is selectively binding, modulating (activating or inhibiting), and/or expanding the target T cells. Thus, for example, the present disclosure provides a method of detecting, in a mixed population of T cells obtained from an individual, the presence of a target T cell that binds an epitope of interest, the method comprising: a) contacting in vitro the mixed population of T cells with a CIIC comprising an epitope of the present disclosure, and b) detecting modulation (activation or inhibition) and/or proliferation of T cells in response to said contacting, wherein modulation and/or proliferation of T cells indicates the presence of the target T cell. Thus, for example, the present disclosure also provides a method of detecting, in a mixed population of T cells obtained from an individual, the presence of a target T cell that binds an epitope of an autoantigen of interest, the method comprising: a) contacting in vitro the mixed population of T cells with a CIIC comprising and presenting an epitope of an autoantigen (e.g., a T1D-epitope or celiac-epitope), and b) detecting modulation (activation or inhibition) and/or proliferation of T cells in response to said contacting, wherein modulation and/or proliferation of T cells indicates the presence of the target T cell. Alternatively, and/or in addition, if activation and/or expansion (proliferation) of the desired T cell population (e.g., T regs) is obtained using a CIIC (e.g., a duplex CIIC), then all or a portion of the population of T cells comprising the activated/expanded T cells can be administered back to the individual as a therapy.

The population of T cells to be targeted by a CIIC may be in vivo in an individual. In such instances, a method for selectively delivering TGF-β (e.g., masked TGF-β) or any other MOD (e.g., wt. or variant IL-2 polypeptides) to an epitope-specific T cell comprises administering the CIIC (e.g., a higher order complex thereof such as a duplex) to the individual.

The MOD sequence(s) present in a CIIC (e.g., a wt. or reduced affinity IL-2 and/or PD-L1 MOD) may be selectively delivered to an epitope-specific T reg cell (sometimes referred to herein as a target regulatory T cell). The action of the CIIC on the T reg cell may, among other things, result in the inhibition or suppression of an autoreactive T cell mediated by the T reg cells.

### I. Dosages

A suitable dosage can be determined by an attending physician, or other qualified medical personnel, based on various clinical factors. As is well known in the medical arts, dosages for any one patient depend upon many factors, including the patient's size, weight, body surface area, age, the particular polypeptide or nucleic acid to be administered, sex of the patient, time, route of administration, general health, and other drugs being administered concurrently.

A CIIC (whether as a single polypeptide chain or as a higher order complex such as a duplex CIIC) may be administered in amounts between 1 ng/kg of body weight and 20 mg/kg of body weight per dose, for example from 0.1 μg/kg of body weight to 1.0 mg/kg of body weight, from 0.1 mg/kg of body weight to 0.5 mg/kg of body weight, from 0.5 mg/kg of body weight to 1 mg/kg of body weight, from 1.0 mg/kg of body weight to 5 mg/kg of body weight, from 5 mg/kg of body weight to 10 mg/kg of body weight, from 10 mg/kg of body weight to 15 mg/kg of body weight, and from 15 mg/kg of body weight to 20 mg/kg of body weight. Doses of CIICs below 0.1 mg/kg of body weight or above 20 mg/kg are envisioned, especially considering the aforementioned factors. Dosage amounts thus include from about 0.1 mg/kg of body weight to about 0.5 mg/kg of body weight, from about 0.5 mg/kg of body weight to 1 mg/kg body weight, from about 1.0 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, and above about 20 mg/kg body weight. A CIIC or a higher order complex thereof (e.g., a duplex CIIC) can also be administered in an amount of from about 0.1 mg/kg of body weight to 5.0 mg/kg of body weight or from about 1 mg/kg of body weight to 50 mg/kg of body weight. A CIIC or a higher order complex thereof (e.g., a duplex CIIC) can be administered in an amount of from about 1 mg/kg of body weight to about 5 mg/kg of body weight or from about 5 mg/kg of body weight to about 10 mg/kg of body weight. A CIIC or a higher order complex thereof (e.g., a duplex CIIC) can be administered in an amount of from about 10 mg/kg of body weight to about 15 mg/kg of body weight or from about 15 mg/kg of body weight to about 20 mg/kg of body weight. If the regimen is a continuous infusion, the dose can also be in the range of 1 μg to 10 mg per kilogram of body weight per minute. Alternatively, continuous administration may be conducted at a rate from about 1 μg to 50 mg per minute or 1 μg to 10 mg per minute.

Those of skill will readily appreciate that dose levels can vary as a function of the CIIC or higher order CIIC complex (e.g., duplex CIIC), including the type and number of MODs per CIIC or higher order CIIC complexes, the severity of the symptoms and the susceptibility of the subject to side effects. Preferred dosages for a given CIIC are readily determinable by those of skill in the art by a variety of means.

In some cases, multiple doses of a CIIC or higher order CIIC complex are administered. The frequency of administration of a CIIC can vary depending on any of a variety of factors, e.g., severity of the symptoms, patient response, etc. For example, in some cases, a CIIC or higher order CIIC complex is administered: (i) less frequently than once per month, e.g., once every two, three, four, six or more months, once per year, or once per month, or (ii) more frequently, e.g., twice per month, three times per month, every other week (qow), once every three weeks, once every four weeks, once per week (qw), twice per week (biw), three times per week (tiw), four times per week, five times per week, six times per week, every other day (qod), or daily (qd).

The duration of administration of a CIIC, e.g., the period of time over which a CIIC is administered, can vary, depending on any of a variety of factors, e.g., patient response, etc. For example, a CIIC can be administered over a period of time ranging from about one day to about one week, from about one week to about four weeks, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more, including continued administration for the patient's life.

Where treatment is of a finite duration, following successful treatment, it may be desirable to have the patient undergo periodic maintenance therapy to prevent recurrence of the disease state, wherein a CIIC is administered in maintenance doses, ranging from those recited above, i.e., 0.1 mg/kg of body weight to about 0.5 mg/kg of body weight, from about 0.5 mg/kg of body weight to about 1 mg/kg of body weight, from about 1.0 mg/kg of body weight to about 5 mg/kg of body weight, from about 5 mg/kg of body weight to about 10 mg/kg of body weight, from about 10 mg/kg of body weight to about 15 mg/kg of body weight, from about 15 mg/kg of body weight to about 20 mg/kg of body weight, and above about 20 mg/kg of body weight. The periodic maintenance therapy can be once per month, once every two months, once every three months, once every four months, once every five months, once every six months, or less frequently than once every six months.

### J. Routes of Administration

A CIIC, or a nucleic acid or recombinant expression vectors comprising nucleic acids encoding one or more polypeptides of a CIIC or its higher order complexes, may be administered to an individual using any available method and route suitable for drug delivery, including in vivo and in vitro methods, as well as systemic and localized routes of administration. A CIIC or higher order CIIC complex can be administered to a host using any available conventional methods and routes suitable for delivery of conventional drugs, including systemic or localized routes. In general, routes of administration contemplated for use in a method include, but are not necessarily limited to, enteral, parenteral, and inhalational routes.

Conventional and pharmaceutically acceptable routes of administration include intramuscular, intratracheal, subcutaneous, intradermal, topical, intravenous, intraarterial (intra-arterial), intralymphatic, rectal, nasal, oral, and other enteral and parenteral routes of administration. Of these, intravenous, intramuscular, and subcutaneous may be more commonly employed. CIICs and nucleic acids and expression vectors encoding them may be administered, for example, intravenously. Routes of administration may be combined, if desired, or adjusted depending upon, for example, the CIIC or higher order CIIC complex and/or the desired effect. A CIIC or higher order CIIC complex can be administered in a single dose or in multiple doses.

A CIIC (e.g., a duplex CIIC) or a nucleic acid or expression vectors comprising nucleic acids encoding one or more polypeptides of a CIIC or its higher order complexes, may also be contacted with cells in vitro. The cells subject to such in vitro treatment and/or their progeny may then be administered to a patient or subject (e.g., the subject from which the cells treated in vitro were obtained.

### K. Subjects Suitable for Treatment

Subjects suitable for treatment with a method employing a CIIC (or nucleic acid encoding a CIIC) disclosed herein include, but are not limited to, those with cancer, infectious diseases (e.g., including those with viral, bacterial, and/or mycoplasma causative agents), allergic reactions, GVHD, HVGD, metabolic disorders, and/or autoimmune diseases (e.g., T1D or celiac disease).

Subjects suitable for treatment who have a cancer include, but are not limited to, individuals who have been provided other treatments for the cancer but who failed to respond to the treatment. Cancers that can be treated with a method include, but are not limited to, those displaying any of the cancer epitopes recited herein including, but not limited to peptide epitopes of AFP, WT-1, HPV, and HBV.

Subjects suitable for treatment with a method employing a CIIC (or nucleic acid encoding a CIIC) include individuals who have an infectious disease include, but are not limited to, individuals who have been provided other treatments for the infectious disease but who failed to respond to the treatment. Infectious diseases that can be treated with a method include, but are not limited to, those having an infectious agent recited herein including, but not limited to, EBV, HPV and HBV epitopes.

Subjects suitable for treatment also include individuals who have an allergy include, but are not limited to, individuals who have been provided other treatments for the allergy, but who failed to respond to the treatment. Allergic reactions that can be treated with a method of the present disclosure, and individuals with such allergic reaction who can be treated, include, but are not limited to, those having an allergy to peanuts, tree nuts, plant pollens, and insect venoms (e.g., hymenoptera proteins including bee and wasp venom proteins such as phospholipase Δ2, melittin, “antigen 5” found in wasp venom, and hyaluronidases).

Subjects suitable for treatment include those with an autoimmune disease or a genetic disposition to develop an autoimmune disease including a family history of an autoimmune disease (e.g., a grandparent, parent, or sibling with the autoimmune disease. The genetic disposition to certain autoimmune diseases, and the serotype/haplotype of some individuals either with or predisposed to those diseases is set forth in, for example, FIG. 17. Subjects suitable for treatment with a method employing a CIIC (or nucleic acid encoding a CIIC) who have an autoimmune disease include, but are not limited to, individuals who have been provided other treatments for the autoimmune disease but who failed to respond to the treatment. Autoimmune diseases that can be treated with a method of the present disclosure include, but are not limited to, Addison's disease, alopecia areata, ankylosing spondylitis, autoimmune encephalomyelitis, autoimmune hemolytic anemia, autoimmune hepatitis, autoimmune-associated infertility, autoimmune thrombocytopenic purpura, bullous pemphigoid, Crohn's disease, Goodpasture's syndrome, glomerulonephritis (e.g., crescentic glomerulonephritis, proliferative glomerulonephritis), Grave's disease, Hashimoto's thyroiditis, inflammatory bowel diseases, irritable bowel disease or syndrome, mixed connective tissue disease, multiple sclerosis, myasthenia gravis (MG), pemphigus (e.g., pemphigus vulgaris), pernicious anemia, polymyositis, psoriasis, psoriatic arthritis, rheumatoid arthritis, scleroderma, Sjögren's syndrome, systemic lupus erythematosus (SLE), vasculitis, and vitiligo. See, e.g., FIG. 17. Additional autoimmune diseases also include any autoimmune disease associated with a self-epitope (autoantigen) described above.

CIICs comprising, for example, one or more masked TGF-β MODs described herein, and/or one or more nucleic acids encoding such CIICs, may be used to treat subjects with metabolic diseases and disorders. Metabolism is the chemical process that the body uses to transform food into the fuel that keeps the body alive. Nutrition (food) consists of proteins, carbohydrates, and fats. These substances are broken down by enzymes in the digestive system, and then carried to the cells where they can be used as fuel. The body either uses these substances immediately, or stores them in the liver, body fat, and muscle tissues for later use. Metabolic disorders, which can be either inherited or acquired, are disorders that interfere with the body's metabolism, and can negatively alter the body's processing and distribution of macronutrients such as proteins, fats, and carbohydrates. Metabolic disorders can happen when abnormal chemical reactions in the body alter the normal metabolic process.

Acquired metabolic disorders, which are metabolic disorders that are acquired during a person's lifetime, can result from a variety of factors. Such disorders include, e.g.: type 2 diabetes (T2D) that can result from insulin resistance and/or deficient insulin secretion; non-alcoholic fatty liver disease (NAFLD) including non-alcoholic steatohepatitis (NASH), which is a severe form of NAFLD that is closely related to obesity, pre-T2D and T2D.

CIICs comprising, for example, masked TGF-β MODs and optionally additional MODs such as the variant IL-2 MODs discussed above, can stimulate the production of T regs and other immune regulatory proteins. Accordingly, such CIICs may be used to treat such inherited and acquired metabolic disorders, including especially T2D and NAFLD such as NASH.

Subjects suitable for treatment with a method employing a CIIC or nucleic acid encoding a CIIC disclosed herein also include individuals who have T1D or celiac disease, including individuals who have been diagnosed as having T1D or celiac disease, and individuals who have been treated for T1D or celiac disease but who failed to respond to the treatment. Suitable subjects also may include individuals who have been diagnosed as being likely to develop T1D or celiac disease or who have symptoms indicating the imminent onset of T1D or celiac disease.

Subjects suitable for treatment with a method employing a CIIC or nucleic acid encoding a CIIC disclosed herein also include those with T1D or a genetic disposition to develop T1D including a family history of T1D (e.g., a grandparent, parent, or sibling with T1D). As discussed above, a number of serotypes have been associated with a substantial risk of developing T1D (e.g., DR3, DR4, DQ2.5 and DQ 8.1), and others with a moderate risk of developing T1D (e.g., DR1, DR8, DR9 and DQ5. Individuals with such serotypes represent subjects suitable for treatment with a method employing a CIIC or nucleic acid encoding a CIIC disclosed herein.

Subjects suitable for treatment include those who have T1D, including individuals who have been diagnosed as having T1D, and individuals who have been treated for T1D but who failed to respond to the treatment who have fasting blood sugars (blood sugar after not eating or drinking for 8 hours) in excess of about 130 mg/dL (or about 140 mg dL), and/or a blood sugar higher than about 180 mg/dL (or about 200 m/dL) 2 hours after a meal (postprandial hyperglycemia). This includes individuals with (i) a genetic disposition to T1D such as a family history of T1D and/or a serotype associated with T1D (e.g., DR4), and (ii) either an elevated fasting blood sugar in excess of 130 or about 140 mg/dL, or postprandial blood sugar in excess of 180 or about 200 mg/dL. See, e.g., www.cdc.gov/diabetes/managing/managing-blood-sugar/bloodglucosemonitoring.html.

Subjects suitable for treatment include those who have T1D, including individuals who have been diagnosed as having T1D, and individuals who have been treated for T1D but who failed to respond to the treatment who have hemoglobin A1C levels from 5.7 to 6.4% (prediabetic levels) or hemoglobin A1C levels above 6.4% (diabetic levels). See, e.g., www.cdc.gov/diabetes/managing/managing-blood-sugar/a1c.html. This includes individuals with both prediabetic or diabetic AiC levels and a genetic disposition to T1D such as a family history of T1D and/or a serotype associated with T1D (e.g., DR4).

Subjects suitable for treatment with a method employing CIIC or nucleic acid encoding a CIIC include those with celiac disease or a genetic disposition to develop celiac disease including a family history of celiac disease (e.g., a grandparent, parent, or sibling with celiac disease). As discussed above, a number of serotypes have been associated with a substantial risk of developing celiac disease including, but not limited to, DQ2, DQ2.2, DQ8, DQ 8.1, and particularly DQ2.5.

Subjects suitable for treatment also include those who have celiac disease, including individuals who have been diagnosed as having celiac disease, and individuals who have been treated for celiac disease, but who failed to respond to the treatment. This includes individuals with a genetic disposition to celiac disease such as a family history of celiac disease, and/or a serotype associated with celiac disease (e.g., those of DQ2.5 or DQ8).

### VII. Certain Aspects

Certain Certain aspects, including embodiments/aspects of the present subject matter described above, may be beneficial alone or in combination with one or more other aspects recited herein below. In addition, while the present subject matter has been disclosed with reference to certain aspects recited below and in the claims, numerous modifications, alterations, and changes to the described aspects/embodiments are possible without departing from the sphere and scope of the present disclosure. Accordingly, it is intended that the present disclosure not be limited to the described embodiments, aspects, and claims, but that it has the full scope defined by the language of this disclosure and equivalents thereof.
1. An MHC Class II protein construct (“CIIC”) comprising in a single aa sequence (e.g., a single polypeptide comprising in the N-terminal to C-terminal direction):
   (i) a peptide epitope aa sequence;
   (ii) an L1 aa linker sequence;
   (iii) an MHC Class II β chain polypeptide sequence (comprising e.g., a β1 and β2 domain sequence);
   (iv) optionally an L2 aa linker sequence;
   (v) an MHC Class II α chain polypeptide sequence (comprising e.g., an α1 and α2 domain sequence);
   (vi) optionally an L3 aa linker sequence;
   (vii) optionally an scaffold sequence and/or MAS;
   (viii) optionally an L4 linker; and
   (ix) optionally one or more additional polypeptide sequences (e.g., one or more affinity sequences and/or targeting sequences);
   wherein
   (i) the CIIC comprises either a body disulfide bond between the β1 domain (e.g., from a cysteine substituted for one of the N-terminal 8 aas of the β1 domain) and the α1 domain (e.g., a cysteine substituted for one of the C-terminal 11 aas of the α1 domain), or a linker disulfide bond between a cysteine in the L1 linker and a cysteine in the α1 domain (e.g., a cysteine substituted for one of the C-terminal 11 aas of the α1 domain); and
(ii) optionally, when the Class II polypeptide comprises a cysteine at aa 43 through aa 48 of the α chain polypeptide sequence (α1 and α2 domain sequence), it is substituted by an aa other than cysteine (e.g., a C47S, C47R or C47K substitution in DQA*05:01).
2. The CIIC of aspect 1 comprising in a single aa sequence in the N-terminal to C-terminal direction:
   (i) a peptide epitope aa sequence;
   (ii) an L1 aa linker sequence;
   (iii) an MHC Class II β chain polypeptide sequence (comprising e.g., a β1 and β2 domain sequence);
   (iv) an L2 aa linker sequence;
   (v) an MHC Class II α chain polypeptide sequence (comprising e.g., an α1 and α2 domain sequence);
   (vi) optionally an L3 aa linker sequence;
   (vii) an optional scaffold sequence and/or MAS;
   (viii) optionally an L4 linker; and
   (ix) optionally one or more additional polypeptide sequences (e.g., one or more affinity sequences and/or targeting sequences).
3. The CIIC of aspect 1 or 2, comprising in a single aa sequence in the N-terminal to C-terminal direction:
   (i) a peptide epitope aa sequence;
   (ii) an L1 aa linker sequence;
   (iii) an MHC Class II β chain polypeptide sequence (e.g., comprising a β1 and β2 domain sequence);
   (iv) optionally an L2 aa linker sequence;
   (v) an MHC Class II α chain polypeptide sequence (e.g., comprising an α1 and α2 domain sequence);
   (vi) an L3 aa linker sequence;
   (vii) a scaffold sequence and/or MAS;
   (viii) optionally an L4 linker; and
   (ix) optionally one or more additional polypeptide sequences (e.g., one or more affinity sequences and/or targeting sequences).
4. The CIIC of any of aspects 1-3, wherein the CIIC optionally comprises a membrane proximal region.
5. The CIIC of any of aspects 1-4 wherein:
   the MHC Class II β chain polypeptide sequence comprises a DPB (e.g., DPB1) β1 and β2 domain sequence; and
   the MHC Class II α chain polypeptide sequence comprises a DPA (e.g., DPA1) α1 and α2 domain sequence.
6. The CIIC of any of aspects 1-5, wherein:
   (i) the MHC Class II β chain polypeptide sequence has at least 90% or 100% aa sequence identity to all or at least 165 contiguous aas of a DPB β1 and β2 domain sequence of DPB1*01:01, DPB1*02:01, DPB1*03:01, DPB1*04:01, DPB1*06:01, DPB1*09:01, DPB1*11:01, DPB1*13:01, DPB1*35:01, DPB1*71:01, DPB1*104:01, or DPB1*141:01, and/or the MHC Class II α chain polypeptide sequence has at least 90% or 100% aa sequence identity to at least 165 contiguous aas of a DPA α1 and α2 domain sequence of DPA1*01:03 or DPA1*02:01; and/or
   (ii) the MHC Class 11 chain polypeptide sequence has at least 90% or at least 95% aa sequence identity to at least 80 or at least 90 contiguous aas of a DPB β1 or β2 domain sequence of DPB1*01:01, DPB1*02:01, DPB1*03:01, DPB1*04:01, DPB1*06:01, DPB1*09:01, DPB1*11:01, DPB1*13:01, DPB1*35:01, DPB1*71:01, DPB1*104:01, or DPB1*141:01, and/or
   the MHC Class II α chain polypeptide sequence has at least 90% or at least 95% aa sequence identity to at least 70 or at least 80 contiguous aas of the DPA α1 or α2 domain sequences of DPA1*01:03 or DPA1*02:01.
7. The CIIC of aspect 6, wherein:
   the at least 90% or 100% aa sequence identity to all or at least 165 contiguous aas of the DPB β1 and β2 domain sequence is at least 95% or at least 98% aa sequence identity; and/or
   the at least 90% or 100% aa sequence identity to at least 165 contiguous aas of the DPA α1 and α2 domain sequence is at least 95% or at least 98% aa sequence identity.
8. The CIIC of any of aspects 5 to 7, comprising a body disulfide bond formed between an N-terminal portion (e.g., the N-terminal 8 amino acids) of the DPB β1 domain and a C-terminal portion (e.g., the C-terminal 6 amino acids) of the DPA α1 domain.
9. The CIIC of aspect 8, wherein the disulfide bond is formed between a cysteine substituted at position 4, 5, 6, or 7 of the DPB β1 domain and a cysteine substituted at position 83, 84, or 85 of the DPA β1 domain.
10. The CIIC of aspect 9 comprising a body disulfide bond formed between a cysteine substituted at position 4, 5, 6, 7, or 8 (e.g., a P4C, E5C, N6C or Y7C) of the DPB β1 domain and a cysteine substituted at position 84 (Q84C) of the DPA α1 domain.
11. The CIIC of any of aspects 5-10, comprising a linker disulfide bond formed between a cysteine in the Li linker sequence and a cysteine at position 77, 78, or 79 of the DPA α1 domain.
12. The CIIC of any of aspects 5-11, comprising a substitution at any one or more of positions 40, 47, 52 or 75 of the DPA α1 domain.
13. The CIIC of aspect 12, wherein position 47 of the DPA α1 domain is a D or an E (e.g., a D40E substitution), and/or position 52 is an H (e.g., a G52H substitution), and/or position 75 is an Ile (e.g., a T401 substitution).
14. The CIIC of any of aspects 5 to 13, wherein:
   (i) aa position 47 of the DPA α1 domain sequence is an aa other than cysteine; or
   (ii) aa position 47 is a serine, lysine, or arginine, or is substituted by a serine, lysine, or arginine.
15. The CIIC of any of aspects 1-4, wherein:
   the MHC Class II β chain polypeptide sequence comprises a DRB (e.g., DRB1, DRB3, DRB4 or DRB5) β1 and β2 domain sequence; and
   the MHC Class II α chain polypeptide sequence comprises a DRA α1 and α2 domain sequence (e.g., DRA*01:01 or DRA*01:02).
16. The CIIC of any of aspects 1-4 or 15, wherein:
   (i) the MHC Class II β chain polypeptide sequence has at least 90% or 100% aa sequence identity to all or at least 170 contiguous aas of a DRB β1 and β2 domain sequence of DRB1*01:01, DRB1*01:02, DRB1*01:03, DRB1*03:01, DRB1*03:02, DRB1*03:04, DRB1*04:01, DRB1*04:02, DRB1*04:03, DRB1*04:04, DRB1*04:05, DRB1*04:06, DRB1*04:08, DRB1*07:01, DRB1*08:01, DRB1*08:02, DRB1*08:03, DRB1*09:01, DRB1*10:01, DRB1*11:01, DRB1*11:03, DRB1*11:04, DRB1*12:01, DRB1*13:01, DRB1*13:03, DRB1*14:01, DRB1*14:02, DRB1*14:05, DRB1*14:06, DRB1*15:01, DRB1*15:02, DRB1*15:03, DRB1*15:04, DRB1*15:05, DRB1*15:06, DRB1*15:07, DRB1*16:01, DRB3*01:01, DRB3*02:01, DRB3*03:01, DRB4*01:01, DRB4*01:03, or
   DRB5*01:01; and/or
   the MHC Class II α chain polypeptide sequence has at least 90% or 100% aa sequence identity to at least 165 contiguous aas of the DRA α1 and α2 domain sequence of DRA1*01:01 or DRA*01:02 or
II) the MHC Class 111 chain polypeptide sequence has at least 90% or at least 95% aa sequence identity to at least 80 or at least 90 contiguous aas of a DRB β1 or β2 domain sequence of DRB1*01:01, DRB1*01:02, DRB1*01:03, DRB1*03:02, DRB1*03:04, DRB1*04:01, DRB1*04:02, DRB1*04:03, DRB1*04:04, DRB1*04:05, DRB1*04:06, DRB1*04:08, DRB1*07:01, DRB1*08:01, DRB1*08:02, DRB1*08:03, DRB1*09:01, DRB1*10:01, DRB1*11:01, DRB1*11:03, DRB1*11:04, DRB1*12:01, DRB1*13:01, DRB1*13:03, DRB1*14:01, DRB1*14:02, DRB1*14:05, DRB1*14:06, DRB1*15:01, DRB1*15:02, DRB1*15:03, DRB1*15:04, DRB1*15:05, DRB1*15:06, DRB1*15:07, DRB1*16:01, DRB3*01:01, DRB3*02:01, DRB3*03:01, DRB4*01:01, DRB4*01:03, or DRB5*01:01, and/or
   the MHC Class II α chain polypeptide sequence has at least 90% or at least 95% aa sequence identity to at least 70 or at least 80 contiguous aas of the DRA α1 or α2 domain sequence of DRA1*01:01 or DRA*01:02.
17. The CIIC of any of aspects 1-4 or 15-16, wherein the MHC Class II β chain polypeptide sequence has at least 90% or 100% aa sequence identity to at least 170 contiguous aas of a DRB β1 and β2 domain sequence of DRB1*01:01, DRB1*03:01, DRB1*04:01, DRB1*04:02, DRB1*04:05, DRB1*08:01, DRB1*09:01, DRB3*01:01, DRB4*01:01, or DRB*05:01.
18. The CIIC of aspect 17, wherein the DRB β1 and β2 domain sequences have at least 90% or 100% aa sequence identity to at least 170 contiguous aas of a DRB β1 and β2 domain sequence of:
   (i) DRB1*01:01 or DRB1*01:02;
   (ii) DRB1*03:01, DRB1*03:02, or DRB1*03:04,
   (iii) DRB1*04:01, DRB1*04:02, DRB1*04:03, DRB1*04:04, DRB1*04:05, DRB1*04:06, or DRB1*04:08;
   (iv) DRB1*08:01, DRB1*08:02, or DRB1*08:03; or
   (v) DRB1*15:01, DRB1*15:02, DRB1*15:03, DRB1*15:04, DRB1*15:05, or DRB1*15:07.
19. The CIIC of any of aspects 15-18, wherein:
   the at least 90% or 100% aa sequence identity to all or at least 170 contiguous aas of a DRB β1 and β2 domain sequence is at least 95% or 100% aa sequence identity; and/or
   the at least 90% or 100% aa sequence identity to at least 165 contiguous aas of the DRA α1 and α2 domain sequence is at least 95% or 100% aa sequence identity.
20. The CIIC of any of aspects 15-19, comprising a body disulfide bond formed between an N-terminal portion (e.g., the N-terminal 8 amino acids) of the DRB β1 domain and a C-terminal portion (e.g., the C-terminal 6 amino acids) of the DRA α1 domain.
21. The CIIC of aspect 20, wherein the body disulfide bond is formed between a cysteine substituted at position 4, 5, 6, or 7 of the DRB β1 domain and a cysteine substituted at position 80, 81, or 82 of the DRA1 α1 domain.
22. The CIIC of aspect 21, comprising a body disulfide bond formed between a cysteine substituted at position 5 (e.g., a P5C) of the DRB β1 domain and a cysteine substituted at position 81 (P81C) of the DRA α1 domain.
23. The CIIC of any of aspects 15-22, comprising a linker disulfide bond formed between a cysteine in the Li linker sequence and a cysteine at position 74, 75, or 76 of the DRA α1 domain.
24. The CIIC of aspect 23, wherein the substitution at position 74, 75 or 76, is a K75C substitution.
25. The CIIC of any of aspects 15-24, comprising a substitution at any one or more of positions 37, 49 or 72 of the DRA α1 domain.
26. The CIIC of any of aspects 15-25, wherein position 37 of the DRA α1 domain is an E (e.g., an Δ37E substitution), and/or position 49 is an H (e.g., a G49H substitution), and/or position 72 is an I (lie).
27. The CIIC of any of aspects 15-26, wherein
   (i) aa position 44 of the DRA α1 domain sequence is an aa other than cysteine; or
   (ii) aa position 44 of the DRA α1 domain is a serine, lysine, or arginine, or is substituted by a serine. lysine, or arginine
28. The CIIC of any of aspects 1-4, wherein:
   the MHC Class II p3 chain polypeptide sequence comprises a DQB (e.g., DQB1 or DQB2) β1 and β2 domain sequence; and
   the MHC Class II α chain polypeptide sequence comprises a DQA (e.g., DQA1 or DQA2) α1 and α2 domain sequence.
29. The CIIC of any of aspects 1-4 or 28 wherein:
   (i) the MHC Class II p3 chain polypeptide sequence has at least 90% or 100% aa sequence identity to all or at least 170 contiguous aas of a DQB β1 and β2 domain sequence of DQB1*02:01, DQB1*02:02, DQB1*03:01, DQB1*03:02, DQB1*03:03, DQB1*03:04, DQB1*04:01, DQB1*04:02, DQB1*05:01, DQB1*06:01, DQB1*06:02, DQB2 isoform 1 or DQB2 isoform 2; and/or
   the MHC Class II α chain polypeptide sequence has at least 90% or 100% aa sequence identity to at least 165 contiguous aas of the DQA α1 and α2 domain sequence of DQA1*01:01, DQA1*01:02, DQA1*01:03, DQA1*01:04, DQA1*02:01, DQA1*03:01, DQA1*03:02, DQA1*04:01, DQA1*05:01, DQA1*05:05, DQA1*06:01, or DQA2*01:01; and/or.
   (ii) the MHC Class 111 chain polypeptide sequence has at least 90% or 95% aa sequence identity to at least 80 or at least 90 contiguous aas of a DQB β1 or β2 domain sequence of DQB1*02:01, DQB1*02:02, DQB1*03:01, DQB1*03:02, DQB1*03:03, DQB1*03:04, DQB1*04:01, DQB1*04:02, DQB1*05:01, DQB1*06:01, DQB1*06:02, DQB2 isoform 1 or DQB2 isoform 2, and/or
   the MHC Class II α chain polypeptide sequence has at least 90% or 95% aa sequence identity to at least 70 or at least 80 contiguous aas of the DQA α1 or α2 domain sequence of DQA1*01:01, DQA1*01:02, DQA1*01:03, DQA1*01:04, DQA1*02:01, DQA1*03:01, DQA1*03:02, DQA1*04:01, DQA1*05:01, DQA1*05:05, DQA1*06:01, or DQA2*01:01.
30. The CIIC of aspect 29, wherein the sequences to which the DQB β1 and β2 domain sequence and the DQA α1 and α2 domain sequence have at least 90% or 100% aa sequence identity are a DQB and DQA allele pair selected from:
   (i) DQB1*02:01 and DQA1*05:01 (DQ2.5);
   (ii) DQB1*02:02 and DQA1*02:01 (DQ2.2);
   (iii) DQB1*03:02 and DQA1*03:01 (DQ8.1);
   (iv) DQB1*04:02 and DQA1*04:01 (DQ4.2);
   (v) DQB1*04:01 or DQB1*04:02 and DQA1*03:01 (DQ4.3a and 4.3b);
   (vi) DQB1*05:01 and DQA1*01:01; or
   (vii) DQB1*06:02 and DQA1*01:02 (DQ6.2).
31. The CIIC of aspect 30, wherein the sequences to which the DQB β1 and β2 domain sequence and the DQA α1 and α2 domain sequence have at least 90% or 100% aa sequence identity are a DQB and DQA allele pair selected from:
   (i) DQB1*02:01 and DQA1*05:01 (DQ2.5); or
   (ii) DQB1*03:02 and DQA1*03:01 (DQ8.1).
32. The CIIC of any of aspects 29-31, wherein:
   the at least 90% or 100% aa sequence identity to all or at least 170 contiguous aas of a DQB β1 and β2 domain sequence is at least 95% or 100% aa sequence identity; and/or
   the at least 90% or 100% aa sequence identity to at least 165 contiguous aas of the DQA α1 and α2 domain sequence is at least 95% or 100% aa sequence identity.
33. The CIIC of any of aspects 29-32, comprising a body disulfide bond formed between the N-terminal portion (e.g., the N-terminal 8 amino acids) of the DQB1 or DQB2 β1 domain and the C terminal portion (e.g., the C-terminal 6 amino acids) of the DQA α1 domain sequence.
34. The CIIC of aspect 33, wherein the body disulfide bond is formed between a cysteine substituted at position 4, 5, 6, or 7 of the DQB1 or DQB2 β1 domain and a cysteine substituted at position 83, 84, or 85 of the DQA1 or DQA2 α1 domain sequence.
35. The CIIC of aspect 34, comprising a body disulfide bond formed between a cysteine substituted at position 5 of the DQB1 (e.g., E5C) or DQB2 (e.g., K5C) β1 domain and a cysteine substituted at position 82, 83, 84 or 85 (e.g., A83C of DQA1*05:01) of the DQA1 or DQA2 α1 domain sequence.
36. The CIIC of any of aspects 28-35, comprising a linker disulfide bond formed between a cysteine in the Li linker sequence and a cysteine substitution at position 76, 77, 78, or 79 of the DQA1 or DQA2 α1 domain sequence.
37. The CIIC of aspect 36, wherein the substitution at position 76, 77, 78, or 79 is a K77C or K78C substitution in DQA1 or an R78C substitution in DQA2.
38. The CIIC of any of aspects 36-37, comprising a substitution at any one or more of positions 40, 52, 74 or 75 of the DQA1 or DQA2 α1 domain.
39. The CIIC of any of aspects 28-38, wherein position 40 of the DQA1 α1 domain is an E (e.g., a G40E substitution), position 52 is an H (e.g., a R52H or S52H substitution), and/or position 74 or 75 is an I (e.g., an S741 substitution in DQA1*05:01 or a F751 substitution in DQA2*01:01).
40. The CIIC of any of aspects 28-39, wherein:
   (i) aa position 47 of the DQA (e.g., DQA1 or DQA2) α1 domain sequence is an aa other than cysteine; or
   (ii) aa position 47 is a serine, lysine, or arginine, or is substituted by a serine, lysine, or arginine.
41. The CIIC of aspects 1-4 or 29-40, wherein the sequences to which the DQB β1 and β2 domain sequence and the DQA α1 and α2 domain sequence have at least 90% or 100% aa sequence identity are DQB1*02:01 and DQA1*05:01 (DQ2.5).
42. The CIIC of aspect 41, wherein the DQB1*02:01 containing sequence comprises the aa sequence RDSPCDFVYQFKGMCYFTNGTERVRLVSRSIYNREEIVRFDSDVGEFRAVTLLGLPAAEYWNSQKDILERKRAA VDRVCRHNYQLELRTTLQRRVEPTVTISPSRTEALNHHNLLVCSVTDFYPAQIKVRWFRNDQEETAGVVSTPLI RNGDWTFQILVMLEMTPQRGDVYTCHVEHPSLQSPITVEW (SEQ ID NO:324), or an aa sequence having at least 90% or at least 95% sequence identity to SEQ ID NO:324; and/or
   wherein the DQA1*05:01 containing sequence comprises the aa sequence EDIVADHVASYGVNLYQSYGPSGQYTHEFDGDEQFYVDLGRKETVWKLPVLHQFRFDPQFALTNIAVLKHNLNI LIKRSNSTCATNEVPEVTVFSKSPVTLGQPNILICLVDNIFPPVVNITWLSNGHSVTEGVSETSFLSKSDHSFFKIS YLTLLPSAEESYDCKVEHWGLDKPLLKHWEPEIPAPMSELTE (SEQ ID NO:325) or an aa sequence having at least 90% or at least 95% sequence identity to SEQ ID NO:325.
43. The CIIC of aspect 42, wherein the CIIC comprises a body disulfide bond between the cysteine at position 5 of the DQB1 sequence and the cysteine at position 83 of the DQA1 sequence.
44. The CIIC of any preceding aspect comprising an L2 linker.
45. The CIIC of any preceding aspect comprising an L3 linker.
46. The CIIC of any preceding aspect comprising an L4 linker.
47. The CIIC of any preceding aspect comprising an Lα linker and/or an Lβ linker.
48. The CIIC of any preceding aspect, wherein each linker present within the CIIC is selected independently.
49. The CIIC of any preceding aspect, wherein at least one linker (e.g., each linker) (i) comprises one or more copies of an aa sequence selected from the group consisting of: GS, GGGS (SEQ ID NO:238), GGGGS (SEQ ID NO:237), AAAGG (SEQ ID NO:248), GGSAAAGGGG (SEQ ID NO:326), and GGSAAAGG (SEQ ID NO:249), any one or more of which may appear 1, 2, 3, 4, 5, 6, 7, 8, 9, or 10 times, or (ii) is a rigid peptide linker.
50. The CIIC of any preceding aspect, wherein when the CIIC comprises a linker disulfide the L1 linker comprises a single cysteine residue.
51. The CIIC of aspect 50, wherein the L1 linker comprises an aa sequence selected from the group consisting of: (CGGGS) (SEQ ID NO:271), (GCGGS) (SEQ ID NO:272), (GGCGS) (SEQ ID NO:273), (GGGCS) (SEQ ID NO:274), and (GGGGC) (SEQ ID NO:275), any one or more of which may appear 1, 2, 3, 4, 5, 6, 7, 8, 9, or 10 times, and wherein the remainder of the linker optionally is comprised of Gly, Ala, and Ser residues.
52. The CIIC of aspect 51 wherein the L1 linker further comprises the aa sequence GGGGS (SEQ ID NO:237), which may appear from 1 to 10 times in repetition.
53. The CIIC of aspect 52, wherein the L1 linker comprises a sequence selected from the group consisting of: GCGASGGGGSGGGGS (SEQ ID NO:276), GCGGSGGGGSGGGGSGGGGS (SEQ ID NO:277), and GCGGSGGGGSGGGGS (SEQ ID NO:278).
54. The CIIC of any preceding aspect, wherein in addition to the β1 and β2 domain sequences the MHC Class II β subunit sequence comprises a membrane proximal region (e.g., placed C-terminal to the β2 domain sequence and N-terminal to the α1 domain sequence, see, e.g., FIG. 1).
55. The CIIC of aspect 54, wherein the membrane proximal region and β2 domain sequences are derived from the same MHC (HLA) 1 subunit allele.
56. The CIIC of any preceding aspect, wherein in addition to the α1 and α2 domain sequence the MHC Class II α subunit sequence comprises a membrane proximal region (e.g., placed C-terminal to the α2 domain sequence). For example, the membrane proximal region may be a sequence having at least about 80% or at least about 90% aa sequence identity to the membrane proximal region of the allele from which the α2 domain sequence was derived, such as 1 or 2 aa insertions, deletions or substitutions.
57. The CIIC of aspect 56, wherein the membrane proximal region and α2 domain sequences are derived from the same MHC (HLA) α subunit allele.
58. The CIIC of any preceding aspect comprising a scaffold sequence and/or a MAS sequence, wherein: (i) the scaffold sequence comprises an interspecific or non-interspecific sequence that forms a duplex or higher order structure; or (ii) the MAS forms a duplex or higher order structure.
59. A higher order CIIC complex (e.g., a duplex CIIC) comprising a first CIIC and a second CIIC of aspect 58.
60. The higher order CIIC complex (e.g., a duplex CIIC) of aspect 59, wherein each CIIC present in the higher order complex (e.g., duplex) comprises a scaffold sequence, and the higher order CIIC complex (e.g., duplex) is formed by binding interactions between the scaffold sequences; and wherein the higher order complex is optionally linked by one or more (e.g., two or more) interchain disulfide bonds between the scaffold sequences.
61. The duplex CIIC of aspect 60, wherein the scaffold sequences of the first CIIC and second CIIC are non-immunoglobulin Fc scaffold sequences that are interspecific or non-interspecific.
62. The duplex CIIC of aspect 60 or 61, wherein the scaffold sequences of the first CIIC and second CIIC are non-immunoglobulin Fc scaffold sequence selected from the group consisting of collectin family domain sequences; coiled-coil domain sequences; leucine-zipper domain sequences; a Fos/Jun domain sequence of a Fos/Jun pair; and an Ig CH1/Ig light chain “Ig C_{L}” (kappa light chain C_{L} K or lambda light chain C_{L} A) domain sequence of a CH1/IgC_{L} pair.
63. The duplex CIIC of aspect 60, wherein the scaffold sequences of the first CIIC and second CIIC are immunoglobulin Fc scaffold sequences optionally linked by one or more (e.g., two or more) interchain disulfide bonds between the scaffold sequences of the first CIIC and the second CIIC.
64. The duplex CIIC of aspect 63, wherein the immunoglobulin Fc scaffold sequences are non-interspecific Ig Fc sequences.
65. The duplex CIIC of aspect 63 or 64, wherein the immunoglobulin Fc scaffold sequences are selected from the group consisting of: IgG1 Fc, IgG2 Fc, IgG3 Fc, IgG4 Fc, IgA Fc, IgD Fc, IgE Fc, and IgM heavy chain constant region (CH2-CH3 or CH2-CH3-CH4) sequences, which may be humanized or of human origin (see FIGS. 2A-2H, SEQ ID NOs:1-13).
66. The duplex CIIC of aspect 63 or 64, wherein the immunoglobulin Fc scaffold sequences comprise an aa sequence having at least about 85% aa sequence identity or at least about 90% aa sequence identity to at least 180 contiguous aas of an Ig Fc region of SEQ ID NOs:1-13 depicted in FIGS. 2A-2H.
67. The duplex CIIC of aspect 63 or 64, wherein the immunoglobulin Fc scaffold sequences comprise an aa sequence having at least about 95% aa sequence identity or 100% aa sequence identity to at least 185 contiguous aas of an Ig Fc region of SEQ ID NOs:1-13 depicted in FIGS. 2A-2H.
68. The duplex CIIC of any of aspects 63 to 67, wherein the immunoglobulin Fc scaffold sequences are IgG1 Fc, IgG2 Fc, IgG3 Fc, or IgG4 Fc aa sequences, which may be humanized or of human origin (see, e.g., FIGS. 2D-2G, SEQ ID NOs:4-12), and wherein when a C-terminal lysine is present in the sequence as transcribed it may be absent due to cellular processing during protein expression.
69. The duplex CIIC of any of aspects 63 to 68, wherein the immunoglobulin Fc scaffold sequences are IgG1 aa sequences (see, e.g., FIG. 2D, SEQ ID NOs:4-8), which may be humanized or of human origin.
70. The duplex CIIC of aspect 69, wherein the immunoglobulin Fc scaffold sequences of each of the first CIIC and second CIIC has at least about 85% aa sequence identity or at least about 90% aa sequence identity to at least 180 contiguous aas of the wt. IgG1 Ig Fc region of SEQ ID NO:4 depicted in FIG. 2D.
71. The duplex CIIC of aspect 69, wherein the immunoglobulin Fc scaffold sequences of each of the first CIIC and second CIIC has at least about 95% aa sequence identity or 100% aa sequence identity to at least 185 contiguous aas of the wt. IgG1 Ig Fc region of SEQ ID NO:4 depicted in FIG. 2D.
72. The duplex CIIC of any of aspects 61 to 71, wherein each immunoglobulin Fc scaffold sequence is an interspecific Ig Fc sequence, optionally linked by one or more (e.g., two or more) interchain disulfide bonds between the scaffold sequences.
73. The duplex CIIC of aspect 72, wherein the scaffold sequences of the first CIIC and second CIIC together comprise a pair of interspecific sequences selected from the group consisting of: knob-in-hole without disulfide (“KiH”); knob-in hole with a stabilizing disulfide bond (“KiHs-s”); HA-TF; ZW-1; 7.8.60; DD-KK; EW-RVT; EW-RVTs-s; and Δ107 interspecific sequence pairs.
74. The duplex CIIC of any of aspects 63 to 72, wherein the immunoglobulin Fc scaffold sequences of the first CIIC and second CIIC comprise one or more substitutions that reduce ADCC, ADCP, and/or CDC relative to an otherwise identical duplex CIIC that does not bear the substitutions (e.g., wt. Fc scaffold sequence).
75. The duplex CIIC of aspect 74, wherein the immunoglobulin Fc scaffold sequences of the first CIIC and second CIIC each comprise an aa sequence having at least 90% aa sequence identity to at least 180 contiguous aas of the wt. IgG1 Fc sequence in FIG. 2D (SEQ ID NO:4) and comprise L234A/L235A substitutions (see, e.g., SEQ ID NO:8).
76. The duplex CIIC of aspect 74, wherein the immunoglobulin Fc scaffold sequences of the first CIIC and second CIIC each comprise an aa sequence having at least 90% aa sequence identity to at least 180 contiguous aas of the wt. IgG1 Fc sequence in FIG. 2D (SEQ ID NO:4) and comprise L234, L235, and P331 substitutions (see, e.g., L234F/L235E/P331S substitutions as in SEQ ID NO:6).
77. The duplex CIIC of any of aspects 63 to 76, wherein, when the immunoglobulin Fc scaffold sequences comprise a sequence having at least about 85% aa sequence identity or at least about 90% aa sequence identity to at least 180 contiguous aas of an Ig Fc region of an IgG1, IgG2, IgG3, or IgG4 (see, e.g., SEQ ID NOs:4-12 depicted in FIGS. 2D-2G), the C-terminal lysine present in the sequence as transcribed may be absent due to cellular processing during protein expression.
78. The CIIC or higher order CIIC complex (e.g., duplex CIIC) of any preceding aspect wherein at least one (e.g., each) CIIC (each CIIC single polypeptide aa sequence) comprises an independently selected membrane association sequence (“MAS”).
79. The CIIC or higher order CIIC complex (e.g., duplex CIIC) of aspect 78, wherein the MAS comprises one or more independently selected transmembrane domains (e.g., one or more transmembrane helices).
80. The CIIC or higher order CIIC complex (e.g., duplex CIIC) of aspect 79, wherein at least one of the independently selected transmembrane domains comprises a glycophorin A or SMIM1 trans membrane domain.
81. The higher order CIIC complex (e.g., duplex CIIC) of aspect 79 or 80, wherein the transmembrane domains form a dimer or other higher order structure resulting in a duplex or other higher order CIIC complex.
82. The CIIC or higher order CIIC complex (e.g., duplex CIIC) of aspect 78, wherein the at least one independently selected MAS comprises one or more amphipathic helices.
83. The CIIC or higher order CIIC complex (e.g., duplex CIIC) of aspect 82, wherein the one or more amphipathic helices are selected from the amphipathic helices of cytidylyltransferase, ADP Ribosylation Factor, blood-clotting factor VIII, vinculin, or DnaA.
84. The CIIC or higher order CIIC complex (e.g., duplex CIIC) of any preceding aspect, wherein at least one (e.g., each) CIIC (each CIIC single polypeptide aa sequence) comprises one or more independently selected additional polypeptides.
85. The CIIC or higher order CIIC complex (e.g., duplex CIIC) of aspect 84, wherein the one or more independently selected additional polypeptides comprise at least one independently selected affinity tag.
86. The CIIC or higher order CIIC complex (e.g., duplex CIIC) of aspect 85, wherein the at least one independently selected affinity tag comprises an aa sequence selected from the group consisting of: hemagglutinin (e.g., YPYDVPDYA SEQ ID NO:315); StrepTags (WSHPQFEK, SEQ ID NO:316), FLAG (e.g., DYKDDDDK, SEQ ID NO:317); c-myc (e.g., EQKLISEEDL, SEQ ID NO:318), RYIRS (SEQ ID NO:319), FHHT (SEQ ID NO:320), WEAAAREACCRECCARA (SEQ ID NO:321), histidine (e.g., HHHHH, SEQ ID NO:322, or HHHHHH, SEQ ID NO:323), glutathione-S-transferase (GST), thioredoxin cellulose binding domains, chitin binding domains, S-peptide, T7 peptide, SH2 domains, C-end RNA tag, inteins, biotin, streptavidin, MyoD, leucine zipper sequences, maltose binding protein, zinc binding domain and calcium binding domain affinity tags.
87. The CIIC or higher order CIIC complex (e.g., duplex CIIC) of aspect 84 or 85, wherein the one or more additional polypeptides comprise at least one independently selected targeting sequence.
88. The CIIC or higher order CIIC complex (e.g., duplex CIIC) of aspect 87, wherein each targeting sequence is an independently selected antibody or antigen binding fragment, or portion thereof; and wherein each targeting sequence (i) is a portion of the single aa sequence of one CIIC (e.g., located C-terminal to the scaffold sequence), or (ii) is covalently (e.g., via a crosslinking agent) or non-covalently linked to at least one single aa sequence of the CIIC or higher order CIIC complex (e.g., duplex CIIC) (e.g., to a side chain of an aa within the at least one single aa sequence, such as via a sulfhydryl of a cysteine).
89. The CIIC or higher order CIIC complex (e.g., duplex CIIC) of aspect 87 or 88, wherein the targeting sequence comprises a scFv or a nanobody.
90. The CIIC or higher order CIIC complex (e.g., duplex CIIC) of aspect 87 or 88, wherein the targeting sequence comprises a bispecific antibody (e.g., a bispecific IgG) that may be humanized having a first antigen binding site directed to a part of the CIIC or higher order CIIC complex (e.g., a scaffold sequence) and a second binding site directed to a cell or tissue target (e.g., an antigen expressed on a cancerous cell or infected cell).
91. The CIIC or higher order CIIC complex (e.g., duplex CIIC) of any of aspects 88 to 90, wherein the antibody or antigen binding fragment/portion thereof, or bispecific antibody second binding site is directed against CD4.
92. The CIIC or higher order CIIC complex (e.g., duplex CIIC) of aspect 91, wherein the antibody or antigen binding fragment is selected from the group consisting of: YTS177, priliximab, keliximab, clenoliximab, zanolimumab, tregalizumab, cedelizumab, ibalizumab, and an antigen binding fragment of any thereof.
93. The CIIC or higher order CIIC complex (e.g., duplex CIIC) of aspect 91, wherein the antibody or antigen binding fragment is ibalizumab or an antigen binding fragment thereof.
94. The CIIC or higher order CIIC complex (e.g., duplex CIIC) of any preceding aspect comprising at least one additional polypeptide, wherein the at least one additional polypeptide comprises at least one post-translational modification sequence.
95. The CIIC or higher order CIIC complex (e.g., duplex CIIC) of aspect 94, wherein at least one post-translational modification sequence comprises a site for attachment of a membrane anchoring lipid.
96. The CIIC or higher order CIIC complex (e.g., duplex CIIC) of aspect 94 or 95, wherein at least one post-translational modification sequence comprises a site for carbohydrate addition.
97. The CIIC or higher order CIIC complex (e.g., duplex CIIC) of any of aspects 84 to 96, wherein at least one of the one or more additional polypeptides is located carboxyl terminal to the α2 domain or a membrane proximal region.
98. The CIIC or higher order CIIC complex (e.g., duplex CIIC) of aspect 97, wherein: (i) at least one of the one or more additional polypeptides is part of an L3 linker, scaffold sequence, or L4 linker; (ii) at least one of the one or more additional polypeptides replaces one or more (e.g., two or more, or all) of the L3 linker, scaffold, and/or
   L4 linker; or (iii) at least one of the one or more additional polypeptides is located on the carboxy terminal to a scaffold sequence to which it may be linked by an L4 linker (see, e.g., FIG. 1).
99. The CIIC or higher order CIIC complex (e.g., duplex CIIC) of any preceding aspect wherein at least one (e.g., each) CIIC single aa sequence (polypeptide) comprises both a scaffold sequence and a targeting sequence.
100. The higher order CIIC complex of any of aspects 59 to 99, wherein: (i) at least one (e.g., each) CIIC single aa sequence (polypeptide) comprises both a scaffold sequence and a targeting sequence; or (ii) each CIIC comprises both a scaffold sequence and a targeting sequence (see, e.g., FIG. 1, structure X).
101. The CIIC or higher order CIIC complex (e.g., duplex CIIC) of any preceding aspect, wherein at least one CIIC single aa sequence (polypeptide) comprises one or more immunomodulatory polypeptide (“MOD”) (e.g., linked to the CIIC by an optional peptide linker and located carboxy terminal to the α2 domain such as between the α2 domain and the scaffold, or carboxy terminal to the scaffold such as in FIG. 1, structures N-X).
102. The CIIC or higher order CIIC complex (e.g., duplex CIIC) of aspect 101, wherein at least one CIIC aa sequence (polypeptide) comprises two or more independently selected MODs optionally placed in tandem.
103. The CIIC or higher order CIIC complex (e.g., duplex CIIC) of aspect 101 or 102, wherein the one or more MOD, or the two or more MODs, is selected independently from the group consisting of: IL-1, IL-2, IL-4, IL-6, IL-7, IL-10, IL-12, IL-15, IL-17, IL-21, IL-23, CD7, CD30L, CD40, CD70, CD80 (B7-1), CD83, CD86 (B7-2), HVEM (CD270), ILT3, ILT4, Fas ligand (FasL), ICAM, ICOS-L, JAG1 (CD339), lymphotoxin beta receptor, 3/TR6, OX40L (CD252), PD-L1, PD-L2, TGF-β1, TGF-β2, TGF-β3, 4-1BBL polypeptide sequences (any or all of which may be human MOD sequences), and variants of any thereof.
104. The CIIC or higher order CIIC complex (e.g., duplex CIIC) of any of aspects 101 to 103, wherein the one or more MOD, or the two or more MODs, is selected independently from the group consisting of: TGF-β, IL-2, PD-L1, IL-10 polypeptide sequences, and variants thereof.
105. The CIIC or higher order CIIC complex (e.g., duplex CIIC) of any of aspects 101 to 104, wherein the one or more MOD, or the two or more MODs, is selected independently from the group consisting of: IL-2, PD-L1, IL-10 polypeptide sequences, and variants of any thereof.
106. The CIIC or higher order CIIC complex (e.g., duplex CIIC) of any of aspects 101 to 105, wherein the one or more MOD, or the two or more MODs, is selected independently from IL-2 and PD-L1 polypeptide sequences. For example, a CIIC or duplex CIIC may comprise (i) at least one or at least two IL-2 MOD (wt. or variant) and/or (ii) at least one or at least two PD-L1 (wt. or variant) polypeptide sequence(s).
107. The CIIC or higher order CIIC complex (e.g., duplex CIIC) of any of aspects 101 to 106, wherein the one or more MOD, or the two or more MODs, comprises at least one IL-2 MOD (wt. or variant) polypeptide sequence, at least two IL-2 MOD (wt. or variant) polypeptide sequences, or at least one pair of IL-2 MOD (wt. or variant) polypeptide sequences optionally located in tandem, wherein each IL-2 MOD (wt. or variant) is selected independently.
108. The CIIC or higher order CIIC complex (e.g., duplex CIIC) of aspect 107, wherein the at least one IL-2 MOD sequence, at least two IL-2 MOD sequences, or at least one pair of IL-2 MOD sequences comprise at least one independently selected variant IL-2 MOD sequence.
109. The CIIC or higher order CIIC complex (e.g., duplex CIIC) of aspect 108, wherein the at least one variant IL-2 MOD sequence comprises an independently selected substitution at one or more, or two or more, of positions 15, 16, 20, 42, 45, 88 and/or 125 of an IL-2 polypeptide sequence having at least 85% (e.g., at least 90%, at least 95%, at least 98%, at least 99%, or 100%) aa sequence identity to at least 100 (e.g., at least 120, at least 130 or at least 133) contiguous aas of SEQ ID NO:208.
110. The CIIC or higher order CIIC complex (e.g., duplex CIIC) of aspect 108 or 109, wherein the at least one variant IL-2 MOD sequence comprises a substitution of F42 and/or H16.
111. The CIIC or higher order CIIC complex (e.g., duplex CIIC) of aspect 110, wherein the at least one variant IL-2 MOD sequence comprises F42A and H16A substitutions, F42T and H16A substitutions, F42A and H16T substitutions, or F42T and H16T substitutions.
112. The CIIC or higher order CIIC complex (e.g., duplex CIIC) of aspect 110, wherein each IL-2 MOD sequence is independently selected and comprises F42A and H16A substitutions, F42T and H16A substitutions, F42A and H16T substitutions, or F42T and H16T substitutions.
113. The CIIC or higher order CIIC complex (e.g., duplex CIIC) of any of aspects 101 to 104, wherein the one or more MOD, or the two or more MODs, comprise at least one or at least two wt. or variant TGF-β MOD sequences.
114. The CIIC or higher order CIIC complex (e.g., duplex CIIC) of aspect 113, wherein the at least one or at least two wt. or variant TGF-β MOD sequences are masked TGF-β MOD sequences comprising a masking polypeptide and a TGF-β polypeptide.
115. The CIIC or higher order CIIC complex (e.g., duplex CIIC) of aspect 114, wherein the masking polypeptide and the TGF-β MOD are located in cis (see, e.g., FIG. 1, structures R and S).
116. The duplex CIIC or other higher order CIIC complex of aspect 114, wherein the masking polypeptide and the TGF-β MOD are located in trans (see e.g., FIG. 1, structures T and U).
117. The CIIC or higher order CIIC complex (e.g., duplex CIIC) of any of aspects 107 to 112 comprising: (i) at least one wt. or variant IL-2 MOD (e.g., having H16 and/or F42 substitutions such as H16A and F42A), or at least two wt. or variant IL-2 MOD sequences; and (ii) at least one TGF-β MOD sequence or masked TGF-β MOD.
118. The CIIC or higher order CIIC complex (e.g., duplex CIIC) of aspect 117, wherein the at least one TGF-β MOD is a masked TGF-β MOD.
119. The CIIC or higher order CIIC complex (e.g., duplex CIIC) of aspect 118, wherein the masking polypeptide and the TGF-β MOD are located in cis (see, e.g., FIG. 1, structures R and S).
120. The CIIC or higher order CIIC complex (e.g., duplex CIIC) of aspect 118, wherein the masking polypeptide and the TGF-β MOD are located in trans (see, e.g., FIG. 1, structures T and U).
121. The CIIC or higher order CIIC complex (e.g., duplex CIIC) of any of aspects 101 to 105, wherein the one or more MOD, or the two or more MODs, comprises at least one or at least two wt. or variant PD-L1 MOD sequences (e.g., CIICs with HLA DR4 and one or more PD-L1 MODs).
122. The CIIC or higher order CIIC complex (e.g., duplex CIIC) of any of aspects 101 to 105, wherein the one or more MOD, or the two or more MODs, comprises at least one or at least two IL-10 MOD (wt. or variant) polypeptide sequences that are selected independently.
123. The CIIC or higher order CIIC complex (e.g., duplex CIIC) of any preceding aspect, wherein the peptide epitope is from 4 aas to about 25 aas (e.g., the epitope can have a length of from 4 aas to 10 aas, from 6 aas to 12 aas, from 8 aas to 20 aas, from 10 aas to 15 aas, from 10 aas to 20 aas, from 15 aas to 20 aas, or from 20 aas to 25 aas); and optionally wherein the peptide epitope sequence is within the N-terminal 10 aas, 15 aas, 20 aas, 25 aas or 30 aas of the at least one (e.g., each) CIIC of the CIIC or higher order CIIC complex.
124. The CIIC or higher order CIIC complex (e.g., duplex CIIC) of any preceding aspect, wherein the peptide epitope is from about 8 aas to about 20 aas.
125. The CIIC or higher order CIIC complex (e.g., duplex CIIC) of any preceding aspect, wherein the peptide epitope is an epitope: of an autoantigen, of a grafted tissue (e.g., HVGD), associated with tissue grafting (e.g., GVHD), of an infectious agent (e.g., a viral or nonviral agent such as a bacterium or fungus), of a cancer-associate antigen, of an allergen, of a T1D-epitope, or of a celiac-epitope.
126. The CIIC or higher order CIIC complex (e.g., duplex CIIC) of any of aspects 1 to 125, wherein the epitope is an epitope of an autoantigen (e.g., an autoantigen set forth herein such as in Section VI.B.6.a).
127. The CIIC or higher order CIIC complex (e.g., duplex CIIC) of aspect 126, wherein the autoantigen is associated with an autoimmune disease selected from the group consisting of: Addison disease, alopecia areata, Addison's anemia, autoimmune hemolytic anemia (AIHA), autoimmune hemolytic anemia, autoimmune hemolytic Donath-Landsteiner anemia, antiphospholipid syndrome, atherosclerosis, autoimmune arthritis, arteriitis temporalis, Takayasu arteriitis, temporal arteriitis/giant cell arteriitis, autoimmune chronic gastritis, autoimmune infertility, autoimmune inner ear disease, Basedow's disease, Bechterew's disease, Behcet's syndrome, autoimmune inflammatory bowel disease, autoimmune cardiomyopathy, idiopathic dilated cardiomyopathy, chronic fatigue immune dysfunction syndrome, chronic inflammatory demyelinating polyneuropathy, chronic polyarthritis, Churg-Strauss syndrome, cicatricial pemphigoid, Cogan syndrome, CREST syndrome, Crohn's disease, dermatitis herpetiformis during, dermatologic autoimmune diseases, dermatomyositis, essential mixed cryoglobulinemia, essential mixed cryoglobulinemia, fibromyalgia, fibromyositis, Goodpasture syndrome, Guillain-Barre syndrome, hematologic autoimmune diseases, Hashimoto thyroiditis, hemophilia, acquired hemophilia, autoimmune hepatitis, idiopathic pulmonary fibrosis, idiopathic thrombocytopenic purpura, Immuno-thrombocytopenic purpura, IgA nephropathy, autoimmune infertility, juvenile rheumatoid arthritis, Lambert-Eaton syndrome, systemic lupus erythematosus, lupus erythematosus, Lyme arthritis, Meniere's disease, mixed connective tissue disease, multiple sclerosis, myasthenia gravis, myositis, polymyositis, neural autoimmune diseases, pemphigus vulgaris, bullous pemphigoid, polyglandular syndrome, polymyalgia rheumatica, primary agammaglobulinemia, primary autoimmune cholangitis, progressive systemic sclerosis, rheumatoid arthritis, sarcoidosis, stiff-man syndrome, Sclerodermia, Scleroderma, Sjögren's syndrome, autoimmune uveitis, and Wegner's disease.
128. The CIIC or higher order CIIC complex (e.g., duplex CIIC) of aspect 126, wherein the autoantigen is associated with Addison's disease, alopecia areata, ankylosing spondylitis, autoimmune encephalomyelitis, autoimmune hemolytic anemia, autoimmune hepatitis, autoimmune-associated infertility, autoimmune thrombocytopenic purpura, bullous pemphigoid, Crohn's disease, Goodpasture's syndrome, glomerulonephritis, Grave's disease, Hashimoto's thyroiditis, mixed connective tissue disease, multiple sclerosis, myasthenia gravis, pemphigus, pernicious anemia, polymyositis, psoriasis, psoriatic arthritis, rheumatoid arthritis, scleroderma, Sjögren's syndrome, systemic lupus erythematosus, vasculitis, or vitiligo. See, e.g., FIG. 17.
129. The CIIC or higher order CIIC complex (e.g., duplex CIIC) of aspect 126, wherein the autoantigens include an epitope of: aggrecan, alanyl-tRNA synthetase (PL-12), alpha beta crystallin, alpha fodrin (Sptan 1), alpha-actinin, α1 antichymotrypsin, α1 antitrypsin, α1 microglobulin, aldolase, aminoacyl-tRNA synthetase, an amyloid, an annexin, an apolipoprotein, aquaporin, bactericidal/permeability-increasing protein (BPI), β-globin precursor BP1, β-actin, β-lactoglobulin A, β-2-glycoprotein I, β2-microglobulin, a blood group antigen, C reactive protein (CRP), calmodulin, calreticulin, cardiolipin, catalase, cathepsin B, a centromere protein, chondroitin sulfate, chromatin, collagen, a complement component, cytochrome C, cytochrome P450 2D6, cytokeratin, decorin, dermatan sulfate, DNA topoisomerase I, elastin, Epstein-Barr nuclear antigen 1 (EBNA1), elastin, entactin, an extractable nuclear antigen, Factor I, Factor P, Factor B, Factor D, Factor H, Factor X, fibrinogen, fibronectin, formiminotransferase cyclodeaminase (LC-1), gp210 nuclear envelope protein, GP2 (major zymogen granule membrane glycoprotein), glycoprotein gpllb/Illa, glial fibrillary acidic protein (GFAP), glycated albumin, glyceraldehyde 3-phosphate dehydrogenase (GAPDH), haptoglobin Δ2, heat shock proteins, hemocyanin, heparin, a histone, histidyl-tRNA synthetase (Jo-1), a hordein, hyaluronidase, immunoglobulins, an integrin, interstitial retinol-binding protein 3, intrinsic factor, Ku (p70/p80), lactate dehydrogenase, laminin, liver cytosol antigen type 1 (LC1), liver/kidney microsomal antigen 1 (LKM1), lysozyme, melanoma differentiation-associated protein 5 (MDAS), Mi-2 (chromodomain helicase DNA binding protein 4), a mitochondrial protein, muscarinic receptors, myelin-associated glycoprotein, myosin, myelin basic protein, myelin proteolipid protein, myelin oligodendrocyte glycoprotein, myeloperoxidase (MPO), rheumatoid factor (IgM anti-IgG), neuron-specific enolase, nicotinic acetylcholine receptor A chain, nucleolin, a nucleoporin, nucleosome antigen, PM/Scl100, PM/Scl 75, pancreatic β-cell antigen, pepsinogen, peroxiredoxin 1, phosphoglucose isomerase, phospholipids, phosphatidyl inositol, platelet derived growth factors, polymerase beta (POLB), potassium channel KIR4.1, proliferating cell nuclear antigen (PCNA), proteinase-3, proteolipid protein, proteoglycan, prothrombin, recoverin, rhodopsin, ribonuclease, a ribonucleoprotein, ribosomes, a ribosomal phosphoprotein, RNA, an Sm protein, Sp100 nuclear protein, SRP54 (signal recognition particle 54 kDa), a selectin, smooth muscle proteins, sphingomyelin, streptococcal antigens, superoxide dismutase, synovial joint proteins, T1F1 gamma collagen, threonyl-tRNA synthetase (PL-7), tissue transglutaminase, thyroid peroxidase, thyroglobulin, thyroid stimulating hormone receptor, transferrin, triosephosphate isomerase, tubulin, tumor necrosis factor-alpha, topoisomerase, U1-dnRNP 68/70 kDa, U1-snRNP A, U1-snRNP C, U-snRNP B/B′, ubiquitin, vascular endothelial growth factor, vimentin, or vitronectin.
130. The CIIC or higher order CIIC complex (e.g., duplex CIIC) of any of aspects 1-125, wherein the peptide epitope is a T1D peptide epitope of a T1D-associated antigen selected from the group consisting of: preproinsulin, proinsulin, insulin, insulin B chain, insulin A chain, 65 kDa isoform of glutamic acid decarboxylase (GAD65), 67 kDa isoform of glutamic acid decarboxylase (GAD67), tyrosine phosphatase (IA-2), heat-shock protein HSP65, islet-specific glucose-6-phosphatase catalytic subunit related protein (IGRP), islet antigen 2 (IA2), and zinc transporter (ZnT8).
131. The CIIC or higher order CIIC complex (e.g., duplex CIIC) of any of aspects 1-125, wherein the peptide epitope is a peptide epitope of a T1D-associated antigen selected from the group consisting of: proinsulin, insulin, GAD65, and islet antigen 2 (IA2).
132. The CIIC or higher order CIIC complex (e.g., duplex CIIC) of any of aspects 1-125, wherein the T1D-epitope is a peptide epitope of the human preproinsulin sequence: MAL WMRLLPLLALLAL WGPDPAAAFVNQHLCGSHLVEALYLVCGERGFFYTPKTRREAEDLQVGQVELGGGP GAGSLQPLALEGSLQKRGIVEQCCTSICSLYQLENYCN (SEQ ID NO:283).
133. The CIIC or higher order CIIC complex (e.g., duplex CIIC) of any of aspects 1-125, wherein the peptide epitope is a peptide epitope of a T1D-associated antigen selected from the group consisting of: GAGSLQPLALEGSLQKRG (SEQ ID NO:284), SLQPLALEGSLQKRG (SEQ ID NO:285), SLQPLALEGSLQSRG (SEQ ID NO:281), QPLALEGSLQKRG (SEQ ID NO:286), and QPLALEGSLQSRG (SEQ ID NO:287).
134. The CIIC or higher order CIIC complex (e.g., duplex CIIC) of any of aspects 1-125, wherein the peptide epitope is a peptide epitope of a T1D-associated antigen selected from the group consisting of: proinsulin 73-90 (GAGSLQPLALEGSLQKR, SEQ ID NO:284), Insulin A (1-15) peptide (GIVDQCCTSICSLYQ, SEQ ID NO:178), Insulin A 1-15 (D4E) peptide: (GIVEQCCTSICSLYQ, SEQ ID NO:327) and the proinsulin peptide SLQPLALEGSLQSRG (SEQ ID NO:281).
135. The CIIC or higher order CIIC complex (e.g., duplex CIIC) of any of aspects 1-125, wherein the peptide epitope is a peptide epitope of a T1D-associated antigen selected from the group consisting of: GAD65 555-567 peptide (NFFRMVISNPAAT, SEQ ID NO:179), GAD65 555-567 (F5571) peptide (NFIRMVISNPAAT, SEQ ID NO:280), and islet antigen 2 (IA2) peptide (SFYLKNVQTQETRTLTQFHF, SEQ ID NO:180).
136. The CIIC or higher order CIIC complex (e.g., duplex CIIC) of any of aspects 1 to 125, wherein the epitope is an epitope of an allergen (e.g., an allergenic protein).
137. The CIIC or higher order CIIC complex (e.g., duplex CIIC) of aspect 136, where the allergen is selected from protein or non-protein components of: nuts (e.g., tree and/or peanuts), glutens, pollens, eggs (e.g., chicken, Gallus domesticus), shellfish, soy, fish, and insect venoms (e.g., bee and/or wasp venom antigens).
138. The CIIC or higher order CIIC complex (e.g., duplex CIIC) of any of aspects 1 to 125, wherein the epitope is associated with GVHD or HVGD.
139. The CIIC or higher order CIIC complex (e.g., duplex CIIC) of aspects 101 to 124, wherein the Class II α chain polypeptide sequence(s) comprise a sequence that has greater than 90% or greater than 95% aa sequence identity to at least 165 contiguous aas of the α1 and α2 domain sequences of:
   (i) DQA1*01:01, DQA1*01:02, or DQA1*01:03 and the epitope is a peptide epitope associated with Multiple Sclerosis (MS), narcolepsy, rheumatoid arthritis, or Systemic Lupus Erythematosus (SLE);
   (ii) DQA1*01:04 and the epitope is a peptide epitope associated with Pemphigus or rheumatoid arthritis;
   (iii) DQA1*03:01 or DQA1*03:02 and the epitope is a peptide epitope associated with rheumatoid arthritis, vasculitis, or vitiligo;
   (iv) DQA1*04:01 and the epitope is a peptide epitope associated with Grave's disease or SLE;
   (v) DQA1*05:01 and the epitope is a peptide epitope associated with Grave's disease, myositis, polymyositis dermatomyositis, or Sjögren's Syndrome; or
   (vi) DQA1*06:01 and the epitope is a peptide epitope associated with Grave's disease.
140. The CIIC or higher order CIIC complex (e.g., duplex CIIC) of any of aspects 1-124, wherein the peptide epitope is a peptide epitope of a celiac-associated antigen [e.g., an epitope of tissue transglutaminases, glutens, gliadins, secalins, hordeins, avenins, and glutenins].
141. The CIIC or higher order CIIC complex (e.g., duplex CIIC) of aspects 1 to 124, wherein the MHC Class II p chain polypeptide sequence(s) comprise a sequence that has greater than 90% or greater than 95% aa sequence identity to at least 165 contiguous aas of the 131 and β2 domain sequences of DPB1*03:01, DPB1*09:01, DPB1*13:01, or DPB1*35:01 and the epitope is a peptide epitope associated with Scleroderma or Systemic Sclerosis.
142. The CIIC or higher order CIIC complex (e.g., duplex CIIC) of aspects 1 to 124, wherein the MHC Class II β chain polypeptide sequence(s) comprise a sequence that has greater than 90% or greater than 95% aa sequence identity to at least 170 contiguous aas of the 131 and β2 domain sequences of:
   (i) DRB1*03:01 and the epitope is a peptide epitope associated with autoimmune hepatitis, Crohn's disease, Multiple Sclerosis, Pemphigus, primary Sjögren's syndrome, SLE; myositis, or Graves' disease;
   (ii) DRB1*04:01 and the epitope is a peptide epitope associated with MS or rheumatoid arthritis;
   (iii) DRB1*04:02 and the epitope is a peptide epitope associated with idiopathic pemphigus vulgaris, or SLE;
   (iv) DRB1*04:03 and the epitope is a peptide epitope associated with SLE;
   (v) DRB1*04:04 or DRB1*04:05 and the epitope is a peptide epitope associated with rheumatoid arthritis or autoimmune hepatitis;
   (vi) DRB1*04:06 or DRB1*04:05 and the epitope is a peptide epitope associated with caspase-8 and silicosis-systemic sclerosis (SSc) or SLE; or
   (vii) one of the DR2 alleles DRB1*1501, DRB1*1502, or DRB1*1503 and the epitope is associated with Crohn's disease, encephalomyelitis, Goodpasture's syndrome, MS, Parkinson's disease, SLE, or ulcerative colitis.
143. The CIIC or higher order CIIC complex (e.g., duplex CIIC) of aspects 1 to 124, wherein the MHC Class II β chain polypeptide sequence(s) comprise a sequence that has greater than 90% or greater than 95% aa sequence identity to at least 170 contiguous aas of the β1 and β2 domain sequences of:
   (i) DQB1*02:01 and the epitope is a peptide epitope associated with autoimmune hepatitis, Grave's disease, myositis, or polymyositis dermatomyositis;
   (ii) DQB1*03:01 and the epitope is a peptide epitope associated with alopecia, pemphigus, rheumatoid arthritis, Sjögren's Syndrome, or vitiligo;
   (iii) DQB1*05:01 or DQB1*05:03 and the epitope is a peptide epitope associated with Grave's Disease, pemphigus, rheumatoid arthritis, vasculitis, or vitiligo.
   (iv) DQB1*06:01 or DQB1*06:02 and the epitope is a peptide epitope associated with encephalomyelitis, MS, Narcolepsy, Parkinson's disease, Sjögren's Syndrome, Scleroderma, or Systemic Sclerosis.
144. The CIIC or higher order CIIC complex (e.g., duplex CIIC) of any of aspects 1 to 143, wherein the CIIC or higher order CIIC complex when expressed by mammalian cells in vitro can accumulate in the culture media to a level (concentrations) from about 25 mg/I to about 350 mg/I of culture media.
145. The CIIC or higher order CIIC complex (e.g., duplex CIIC) of aspect 144, wherein the CIICs can accumulate to a level from about 25 mg/I to about 50 mg/I or from about 50 mg/I to about 100 mg/I.
146. The CIIC or higher order CIIC complex (e.g., duplex CIIC) of aspect 144, wherein the CIIC or higher order CIIC complex can accumulate to a level from about 100 mg/I to about 150 mg/I or from about 150 mg/I to about 200 mg/I.
147. The CIIC or higher order CIIC complex (e.g., duplex CIIC) of aspect 144, wherein the CIIC or higher order CIIC complex can accumulate to a level from about 200 mg/I to about 250 mg/I, or from about 250 mg/I to about 300 mg/I.
148. The CIIC or higher order CIIC complex (e.g., duplex CIIC) of aspect 144, wherein the CIIC or higher order CIIC complex can accumulate to a level from about 300 mg/I to about 325 mg/I, or from about 325 mg to about 350 mg/I (e.g., 330 mg/I).
149. The CIIC or higher order CIIC complex (e.g., duplex CIIC) of any of aspects 144 to 148, wherein the CIIC is expressed in CHO cells.
150. The CIIC or higher order CIIC complex (e.g., duplex CIIC) of any of aspects 144 to 149, wherein the CIIC is a soluble duplex CIIC or other higher order CIIC complex.
151. The soluble duplex CIIC or other higher order CIIC complex of aspect 150, wherein the soluble duplex CIIC or other higher order CIIC comprises a scaffold.
152. The soluble duplex CIIC or other higher order CIIC complex of aspect 151, wherein the scaffold comprises an Ig Fc polypeptide.
153. The soluble duplex CIIC or other higher order CIIC complex of aspect 152, wherein the scaffold comprises an IgG Fc polypeptide, optionally comprising one or more substitutions (e.g., LALA substitutions such as in FIG. 2D) that reduce CDC, ADCP, and/or ADCC relative to the level of CDC or ADCC induced by an otherwise identical soluble duplex CIIC or other higher order CIIC complex that lacks the substitutions.
154. The soluble duplex CIIC or other higher order CIIC complex of aspect 153, wherein the soluble duplex CIIC or other higher order CIIC complex has a structural organization as depicted in FIG. 1, structures H, I, or O to X.
155. The CIIC or higher order CIIC complex (e.g., duplex CIIC) of any preceding aspect, wherein less than 10% of the CIIC, duplex CIIC, or other higher order CIIC complex is lost to denaturation and/or aggregation when subject to freezing at about −80° C. and thawing at about 20° C. based on size separation chromatographic analysis.
156. The CIIC or higher order CIIC complex (e.g., duplex CIIC) of any preceding aspect, wherein less than 10% of the CIIC, duplex CIIC, or other higher order CIIC complex is lost to denaturation and/or aggregation when subject to two cycles or three cycles of freezing at about −80° C. and thawing at about 20° C. based on size separation chromatographic analysis.
157. The CIIC, duplex CIIC, or other higher order CIIC complex of any preceding aspect, wherein less than 5% or less than 3% of the CIIC, duplex CIIC, or other higher order CIIC complex is lost to denaturation and/or
   aggregation when subject to freezing at about −80° C. and thawing at about 20° C. based on size separation chromatographic analysis (e.g., size separation chromatographic analysis initiated within 10 minutes of thawing).
158. The CIIC, duplex CIIC, or other higher order CIIC complex of any preceding aspect, wherein less than 5% or less than 3% of the CIIC, duplex CIIC, or other higher order CIIC complex is lost to denaturation and/or
   aggregation when subject to two cycles or three cycles of freezing at about −80° C. and thawing at about 20° C. based on size separation chromatographic analysis (e.g., size separation chromatographic analysis initiated within 10 minutes of thawing).
159. The CIIC or higher order CIIC complex (e.g., duplex CIIC) of any preceding aspect, wherein the proteins are substantially stable to heating in a phosphate buffered saline solution (PBS plus saline pH 7.4, see e.g., Example 4 for the composition of PBS plus saline) at or about 37° C. (e.g., for 1 minute, 1 hour, or one day).
160. The CIIC or higher order CIIC complex (e.g., duplex CIIC) of any preceding aspect, wherein the CIIC or higher order complex displays resistance to thermal denaturation when heated to 40° C. or 42° C. in PBS plus saline (e.g., for 1 minute, 1 hour, or one day). See, e.g., Example 4 for the composition of PBS plus saline.
161. The CIIC or higher order CIIC complex (e.g., duplex CIIC) of any preceding aspect, wherein the CIIC or higher order CIIC complex displays resistance to thermal denaturation when heated to 42° C. in PBS plus saline for 24 hours or more (one day or more).
162. The CIIC or higher order CIIC complex (e.g., duplex CIIC) of any preceding aspect, wherein less than 20% or less than 15% of the protein is lost (e.g., due to aggregation or degradation) after 1 day or after 5 days at 42° C. based on size separation chromatographic analysis (e.g., using integration of the peak areas with detection at 280 nm).
163. The CIIC or higher order CIIC complex (e.g., duplex CIIC) of any preceding aspect, wherein less than 20% or less than 15% of the protein is lost (e.g., due to aggregation or degradation) after 7 days or after 10 days at 42° C. based on size separation chromatographic analysis.
164. The CIIC or higher order CIIC complex (e.g., duplex CIIC) of any preceding aspect, wherein less than 10% or less than 5% of the protein is lost (e.g., due to aggregation or degradation) after 1 day or after 5 days based on size separation chromatographic analysis.
165. The CIIC or higher order CIIC complex (e.g., duplex CIIC) of any preceding aspect, wherein less than 10% or less than 5% of the protein is lost (e.g., due to aggregation or degradation) after 7 days or after 10 days at 42° C. based on size separation chromatographic analysis.
166. The CIIC or higher order CIIC complex (e.g., duplex CIIC) of any preceding aspect, wherein the temperature at which aggregation of the CIIC or higher order CIIC complex (e.g., duplex CIIC) begins (initiates, Tagg) is from about 42 to about 45° C. or from about 45 to about 50° C. (e.g., 45.1 to 49.6° C.) based on light scattering when measured in PBS plus saline pH 7.4 using the attenuation of transmitted light due to scattering to detect aggregate formation in a Nanotemper Prometheus or similar instrument.
167. The CIIC or higher order CIIC complex (e.g., duplex CIIC) of any preceding aspect, wherein the temperature at which aggregation of the CIIC or higher order CIIC complex (e.g., duplex CIIC) begins (initiates, Tagg) is from about 50 to about 55° C., or is from about 55 to about 60° C. based on light scattering when measured in PBS plus saline pH 7.4 (see e.g., Example 4) using the attenuation of transmitted light due to scattering to detect aggregate formation in a Nanotemper Prometheus or similar instrument.
168. The CIIC or higher order CIIC complex (e.g., duplex CIIC) of any preceding aspect, wherein the temperature at which aggregation of the CIIC or higher order CIIC complex (e.g., duplex CIIC) begins (initiates, Tagg) is from about 60 to about 65° C., or is from about 65 to about 70° C. (e.g., 68.5° C.) based on light scattering when measured in PBS plus saline pH 7.4 (see e.g., Example 4) using the attenuation of transmitted light due to scattering to detect aggregate formation in a Nanotemper Prometheus or similar instrument.
169. The CIIC or higher order CIIC complex (e.g., duplex CIIC) of any preceding aspect, wherein the first melting point of the class II MHC sequence (Tm1) is greater than about 40° C. or greater than about 60° C., and wherein when an IgG scaffold is present as a duplex, the melting point of the scaffold sequence may be greater than about 70° C. or greater than about 80° C.
170. The CIIC or higher order CIIC complex of any of aspects 1-60, 63-77, and 84-169, wherein: the CIIC is a soluble duplex CIIC comprising a first CIIC and a second CIIC, where each of the first CIIC and the second CIIC comprises an Ig Fc scaffold;
   the duplex is formed by interactions between the Ig Fc scaffold sequences; and
   at least one (e.g., each) of the first CIIC and the second CIIC comprises one or more MODs or two or more MODs.
171. The CIIC or higher order CIIC complex of aspect 170, wherein the Ig Fc sequence of the first and the second CIIC are interspecific sequences.
172. A pharmaceutical composition comprising one or more CIICs or higher order CIIC complexes (e.g., duplexes) of any preceding aspect.
173. A nucleic acid or recombinant expression vector comprising a nucleic acid sequence encoding one or more CIICs or higher order CIIC complexes (e.g., duplexes) of any preceding aspect.
174. Two or more nucleic acids or recombinant expression vectors of aspect 173, wherein each of the two or more nucleic acids or recombinant expression vectors comprises a nucleic acid sequence encoding a CIIC of any of aspects 1 to 171.
175. A pharmaceutical composition comprising one or more nucleic acids or recombinant expression vectors of aspect 173 or 174.
176. A method of treatment or prophylaxis of a patient or subject having a disease or condition (e.g., cancer, allergy, autoimmunity [other than or in addition to T1D and/or celiac disease], T1D, celiac disease, GVHD, HGVD, infection, or a metabolic disorder such as T2D or an NAFLD such as NASH) comprising:
   (i) administering to a patient/subject (e.g., a patient in need thereof) an effective amount of one or more CIICs or higher order CIIC complexes (e.g., duplexes) of any of aspects 1 to 171, or a pharmaceutical composition of aspect 172;
   (ii) administering to a patient/subject (e.g., a patient in need thereof) an effective amount of one or more nucleic acids or recombinant expression vectors encoding one or more CIICs or higher order CIIC complexes (e.g., a duplexes) of any of aspects 1 to 171;
   (iii) contacting a cell or tissue, either in vitro or in vivo, with one or more CIICs or higher order CIIC complexes (e.g., a duplexes) of any of aspects 1 to 171, and administering the cell, tissue, or progeny thereof to the patient/subject; or
   (iv) contacting a cell or tissue, either in vitro or in vivo, with one or more nucleic acids or recombinant expression vectors encoding one or more CIICs or higher order CIIC complexes (e.g., a duplexes) of any of aspects 1 to 171, and administering the cell, tissue, or progeny thereof to the patient/subject.
177. A method of treatment or prophylaxis of a patient or subject having a disease or condition (e.g., an allergy or autoimmunity) comprising administering to a patient/subject (e.g., a patient in need thereof):
   (i) one or more CIICs or higher order CIIC complexes (e.g., duplexes) of any of aspects 1 to 169;
   (ii) one or more nucleic acids or recombinant expression vectors encoding one or more CIICs or higher order CIIC complexes (e.g., a duplex) of aspect 173 or 174; or
   (iii) a pharmaceutical composition of aspect 175.
178. A method of treatment or prophylaxis of a patient or subject having T1D or celiac disease comprising administering to a patient/subject (e.g., a patient in need thereof):
   (i) one or more CIICs or higher order CIIC complexes (e.g., duplexes) of any of aspects 1 to 171;
   (ii) one or more nucleic acids or recombinant expression vectors encoding one or more CIICs or higher order CIIC complexes (e.g., duplexes); or
   (iii) a pharmaceutical composition of aspect 172 or 175.
179. The method of aspect 177 or 178, wherein the one or more CIICs or higher order CIIC complexes (e.g., a duplex) further comprise at least one targeting sequence (e.g., a targeting sequence specific for an antigen associated with a cell or tissue).
180. The method of any of aspects 176 to 179, wherein the one or more CIICs or higher order CIIC complexes (e.g., duplexes) are administered to a mammalian patient or subject.
181. The method of any of aspects 176 to 180, wherein the subject is human.
182. The method of any of aspects 176 to 180, wherein the subject is non-human (e.g., rodent, lagomorph, bovine, canine, feline, rodent, murine, caprine, simian, ovine, equine, lappine, porcine, etc.).
183. The method of any of aspects 176 to 182, wherein the disease or condition is an autoimmune disease, and the epitope is an epitope of an autoantigen, and wherein at least one (e.g., each) of the one or more CIICs or higher order CIIC complexes (e.g., a duplex) optionally comprises a targeting sequence to direct the at least one of the one or more CIICs or higher order CIIC complexes (e.g., a duplex) to a tissue affected by the autoimmune disease (e.g., an autoimmune disease or an autoimmune disease associated with a self-epitope (autoantigen) set forth herein such as in Section VI.B.6.a).
184. The method of any of aspects 176 to 183, wherein at least one (e.g., each) of the one or more CIICs or higher order CIIC complexes (e.g., a duplex) optionally comprises a targeting sequence to direct the at least one of the one or more CIICs or higher order CIIC complexes (e.g., a duplex) to a tissue affected by celiac disease (e.g., gut, intestine, or small intestine) or T1D (e.g., pancreas such as the islet tissue or beta cells).
185. The method of any of aspects 176 to 183, wherein the autoimmune disease is selected from the group consisting of: Addison's disease, alopecia areata, ankylosing spondylitis, autoimmune encephalomyelitis, autoimmune hemolytic anemia, autoimmune hepatitis, autoimmune-associated infertility, autoimmune thrombocytopenic purpura, bullous pemphigoid, Crohn's disease, Goodpasture's syndrome, glomerulonephritis (e.g., crescentic glomerulonephritis, proliferative glomerulonephritis), Grave's disease, Hashimoto's thyroiditis, autoimmune gastritis, inflammatory bowel diseases, irritable bowel disease or syndrome, mixed connective tissue disease, multiple sclerosis, myasthenia gravis (MG), pemphigus (e.g., pemphigus vulgaris), pernicious anemia, polymyositis, psoriasis, psoriatic arthritis, rheumatoid arthritis, scleroderma, Sjögren's syndrome, systemic lupus erythematosus (SLE), vasculitis, and vitiligo.
186. The method of aspect 185, wherein the autoimmune disease is autoimmune gastritis (e.g., autoimmune chronic gastritis).
187. The method of any of aspects 176-183, wherein the patient or subject has celiac disease, and wherein, prior to the steps of administering and/or contacting, the patient or subject is screened to determine (i) their MHC Class II haplotype or genotype, and/or (ii) at least one celiac-epitope for which the patient or subject has epitope specific T-cells (e.g., effector T cells and/or T regs).
188. The method of aspect 187, wherein the CIIC or higher order CIIC complex comprises an MHC Class II α chain polypeptide sequence and an MHC Class II β chain polypeptide sequence consistent with the patient's genotype, and comprises a celiac-epitope for which the patient or subject has T-cells specific to the celiac-epitope of the CIIC or higher order CIIC complex.
189. The method of aspect 187 or 188, wherein:
   (i) the MHC Class II α chain polypeptide sequence has at least 95% or 98% sequence identity to DQA1*05:01 and the MHC Class II β chain polypeptide sequence has at least 95% or 98% sequence identity to the β1 and 12 domains of DQB1*02:01;
   (ii) the MHC Class II α chain polypeptide sequence has at least 95% or 98% sequence identity to the α1 and α2 domains of DQA1*03:01 and the MHC Class II β chain polypeptide sequence has at least 95% or 98% sequence identity to the β1 and β2 domains of DQB1*03:02; or
   (iii) the patient or subject is of the DQ5 serotype.
190. The method of any of aspects 176 to 184, wherein the patient or subject has T1D disease, and wherein, prior to the steps of administering and/or contacting, the patient or subject is screened to determine (i) their MHC Class II haplotype or genotype, and/or (ii) at least one T1D-epitope for which the patient or subject has epitope specific T-cells (e.g., effector T cells and/or T regs).
191. The method of aspect 190, wherein the CIIC or higher order CIIC complex comprises an MHC Class II α chain polypeptide sequence and an MHC Class II β chain polypeptide sequence consistent with the patient's genotype, and comprises a T1D-epitope for which the patient or subject has T-cells specific to the TID-epitope of the CIIC or higher order CIIC complex.
192. The method of aspect 190 or 191, wherein:
   (i) the MHC Class II α chain polypeptide sequence has at least 95% or 98% sequence identity to DQA1*05:01 and the MHC Class II β chain polypeptide sequence has at least 95% or 98% sequence identity to the β1 and 12 domains of DQB1*02:01;
   (ii) the MHC Class II α chain polypeptide sequence has at least 95% or 98% sequence identity to the α1 and α2 domains of DQA1*03:01 and the MHC Class II β chain polypeptide sequence has at least 95% or 98% sequence identity to the β1 and β2 domains of DQB1*03:02;
   (iii) the MHC Class II α chain polypeptide sequence has at least 95% or 98% sequence identity to the α1 and α2 domains of DQA1*01:01 and the MHC Class II β chain polypeptide sequence has at least 95% or 98% sequence identity to the β1 and β2 domains of DQB1*05:01;
   (iv) the MHC Class II α chain polypeptide sequence has at least 95% or 98% sequence identity to the α1 and α2 domains of DQA1*04:01 and the MHC Class II β chain polypeptide sequence has at least 95% or 98% sequence identity to the β1 and β2 domains of DQB1*04:02; or
   (v) the MHC Class II α chain polypeptide sequence has at least 95% or 98% sequence identity to the α1 and α2 domains of DQA1*03:01 and an MHC Class II β chain polypeptide sequence has at least 95% or 98% sequence identity to the β1 and β2 domains of DQB1*03:03.
193. The method of aspect 190 or 191, wherein the subject is of the DQ5, DR3, DR4, DR1, DR8, or DR9 serotype.
194. The method of any of aspect 176-184, or 190-193, wherein the CIIC or higher order CIIC complex (e.g., duplex) reduces hemoglobin Δ1C or blood sugar (e.g., glucose) levels in a diabetic or prediabetic patient/subject relative to the hemoglobin Δ1C or blood sugar levels prior to administration of the CIIC or duplex CIIC.
195. The method of any of aspects 176 to 194, wherein the one or more CIIC or higher order CIIC complex (e.g., a duplex) comprises:
   an MHC Class II α lpha chain polypeptide having an α1 and α2 domain sequence and/or
   an MHC Class II beta chain polypeptide having a β1 and β2 domain sequence correlated with an autoimmune disease set forth in FIG. 17.
196. The method of any of aspects 190 to 193, wherein the at least one (e.g., each) of the one or more CIICs or higher order CIIC complexes (e.g., a duplex) comprises an epitope of an autoantigen associated with an autoimmune disease set forth in FIG. 17.
197. The method of any of aspects 176 to 182, wherein the disease or condition is an allergy, and the epitope is an epitope of an allergen (e.g., protein allergen).
198. The method of aspect 197, wherein the allergen is selected from: peanuts, tree nuts, plant pollens, and hymenoptera proteins (e.g., allergens in bee and wasp venoms such as phospholipase Δ2, melittin, “antigen 5” found in wasp venom, and hyaluronidases) 199. The method of aspect 198, wherein the allergen is a peanut allergen, and the epitope is selected from PGQFEDFF (SEQ ID NO:328), YLQGFSRN (SEQ ID NO:329), FNAEFNEIRR (SEQ ID NO:330), QEERGQRR (SEQ ID NO:331), DITNPINLRE (SEQ ID NO:332), NNFGKLFEVK (SEQ ID NO:333), GNLELV (SEQ ID NO:334), RRYTARLKEG (SEQ ID NO:335), ELHLLGFGIN (SEQ ID NO:336), HRIFLAGDKD (SEQ ID NO:337), IDQIEKQAKD (SEQ ID NO:338), KDLAFPGSGE (SEQ ID NO:339), KESHFVSARP (SEQ ID NO:340), NEGVIVKVSKEHVEELTKHAKSVSK (SEQ ID NO:341), HASARQQWEL (SEQ ID NO:342), QWELQGDRRC (SEQ ID NO:343), DRRCQSQLER (SEQ ID NO:344), LRPCEQHLMQ (SEQ ID NO:345), KIQRDEDSYE (SEQ ID NO:346), YERDPYSPSQ (SEQ ID NO:347), SQDPYSPSPY (SEQ ID NO:348), DRLQGRQQEQ (SEQ ID NO:349), KRELRNLPQQ (SEQ ID NO:350), QRCDLDVESG (SEQ ID NO:351), IETWNPNNQEFECAG (SEQ ID NO:352), GNIFSGFTPEFLAQA (SEQ ID NO:353), VTVRGGLRILSPDRK (SEQ ID NO:354), and DEDEYEYDEEDRRRG (SEQ ID NO:355).
200. The method of any of aspects 176 to 199, further comprising administering an NSAID (e.g., Cox-1 and/or Cox-2 inhibitors such as celecoxib, diclofenac, diflunisal, etodolac, ibuprofen, indomethacin, ketoprofen, and naproxen).
201. The method of any of aspects 176 to 200, further comprising administering a corticosteroid (e.g., cortisone, dexamethasone, hydrocortisone, betamethasone, fludrocortisone, methylprednisolone, prednisone, prednisolone, and triamcinolone) before, during (concurrent or combined administration) or subsequent to the administration of the CIICs, duplex CIICs, or other higher order CIIC complex(es).
202. The method of any of aspects 176 to 201, further comprising administering an agent that blocks one or more actions of tumor necrosis factor alpha (e.g., an anti-TNF alpha such as golimumab, infliximab, certolizumab, adalimumab or a TNF alpha decoy receptor such as etanercept) (subject to the proviso that the one or more CIICs or higher order CIIC complexes (e.g., a duplex) do not comprise a tumor necrosis factor alpha MOD or variant MOD and/or an aa sequence to which the agent binds).
203. The method of any of aspects 176 to 202, further comprising administering one or more agents that bind to an IL-1 receptor competitively with IL-1 (e.g., anakinra) (subject to the proviso that the one or more CIICs or higher order CIIC complexes (e.g., a duplex) do not comprise an IL-1 MOD or variant MOD and/or an aa sequence to which the agent binds).
204. The method of any of aspects 176 to 203, further comprising administering one or more agents that bind to an IL-6 receptor and inhibit IL-6 from signaling through the receptor (e.g., tocilizumab) (subject to the proviso that one or more CIICs or higher order CIIC complexes (e.g., a duplex) do not comprise an IL-6 MOD or variant MOD and/or an aa sequence to which the agent binds).
205. The method of any of aspects 176 to 204, further comprising administering one or more agents that bind to CD80 and/or CD86 receptors and inhibit T cell proliferation and/or B cell immune response (e.g., abatacept) (subject to the proviso that the one or more CIICs or higher order CIIC complexes (e.g., a duplex) do not comprise a CD80 and/or CD86 MOD or variant MOD and/or an aa sequence to which the agent binds).
206. The method of any of aspects 176 to 205, further comprising administering one or more agents that bind to CD20 resulting in B-Cell death (e.g., rituximab) (subject to the proviso that the one or more CIICs or higher order CIIC complexes (e.g., a duplex) do not comprise a CD20 MOD or variant MOD, and/or an aa sequence to which the agent binds).
207. The method of any of aspects 176 to 206, wherein the one or more CIICs or higher order CIIC complexes (e.g., a duplex), or the one or more nucleic acids or recombinant expression vectors encoding the one or more CIICs or higher order CIIC complexes (e.g., a duplex) are administered in a composition comprising at least one pharmaceutically acceptable excipient.
208. The method of any of aspects 176 to 207, wherein the administering to a patient or subject comprises administering one or more doses comprising from about 1 ng/kg of body weight to about 20 mg/kg of body weight per dose (e.g., from 0.1 μg/kg of body weight to 1.0 mg/kg of body weight, from 0.1 mg/kg of body weight to 0.5 mg/kg of body weight, from 0.5 mg/kg of body weight to 1 mg/kg of body weight, from 1.0 mg/kg of body weight to 5 mg/kg of body weight, from 5 mg/kg of body weight to 10 mg/kg of body weight, from 10 mg/kg of body weight to 15 mg/kg of body weight, and from 15 mg/kg of body weight to 20 mg/kg of body weight of one or more CIICs or higher order CIIC complexes (e.g., duplexes). For example, the dose may be from about 0.5 to about 10 mg/kg, or from about 10 to about 20 mg/kg of body weight.
209. The method of aspect 208, wherein the dose of the one or more CIICs or higher order CIIC complexes (e.g., a duplexes) is from about 0.1 to 5 mg/kg of body weight or from about 5 to about 15 mg/kg of body weight.
210. The method of aspect 208, wherein the dose of the one or more CIICs or higher order CIIC complexes (e.g., a duplexes) is from about 1 mg/kg of body weight to 5 mg/kg of body weight or from about 5 to about 10 mg/kg of body weight.
211. The method of any of aspects 176 to 210, wherein the administration is conducted: (i) less frequently than once per month (e.g., once every two, three, four, six or more months, once per year, or once per month); or (ii) more frequently than one per month (e.g., twice per month, three times per month, every other week (qow), one every three weeks, once every four weeks, once per week (qw), twice per week (biw), three times per week (tiw), four times per week, five times per week, six times per week, every other day (qod), daily (qd), twice a day (qid), or three times a day (tid)).
212. The method of any of aspects 176 to 211, wherein duration of administration ranges from about one day to about one week, from about one week to about four weeks, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more, including continued administration for the patient's life.
213. The method of any of aspects 176 to 212, wherein duration of administration ranges from about one month to about 1 year.
214. The method of any of aspects 176 to 213, wherein the patient or subject is administered one or more maintenance doses each comprising one or more CIICs or higher order CIIC complexes (e.g., a duplexes), ranging from 0.1 mg/kg of body weight to about 0.5 mg/kg of body weight, from about 0.5 mg/kg of body weight to about 1 mg/kg of body weight, from about 1.0 mg/kg of body weight to about 5 mg/kg of body weight, from about 5 mg/kg of body weight to about 10 mg/kg of body weight, from about 10 mg/kg of body weight to about 15 mg/kg of body weight, from about 15 mg/kg of body weight to about 20 mg/kg of body weight, and above about 20 mg/kg of body weight.
215. The method of aspect 214, wherein the maintenance dose is administered once per month, once every two months, once every three months, once every four months, once every five months, once every six months, or less frequently than once every six months.
216. The method of any of aspects 176 to 215, wherein when at least one (e.g., each) of the one or more CIICs or higher order CIIC complexes (e.g., a duplexes) is administered continuously, it is administered at a rate from about 1 μg to 10 mg per kilogram of body weight per minute.
217. The method of any of aspects 176 to 216, wherein when any one or more CIICs or higher order CIIC complexes (e.g, duplexes) is administered continuously, it is administered at a rate from about 1 μg to 1 mg per minute.
218. The method of any of aspects 176 to 217, the administering or contacting is conducted by a route selected from the group consisting of: intramuscular, intralymphatically, intratracheal, intracranial, subcutaneous, intradermal, topical, intravenous, intraarterial, rectal, nasal, and oral.
219. The method of any of aspects 176 to 218, wherein the administering is conducted intravenously.
220. The use of one or more CIICs or higher order CIIC complexes (e.g., a duplexes) of aspects 1-171, for use as a medicament.
221. The use of one or more CIICs or higher order CIIC complexes (e.g., a duplexes) of aspects 1-171 in the treatment: (i) of an autoimmune disease other than, or in addition to, celiac disease and/or T1D; (ii) an allergy (e.g., an allergic reaction to a specific antigen); (iii) a metabolic disease; (iv) an infections by a viral agent; (v) an infections by a non-viral agent; (vi) of GVHD; (vii) of HVGD; (viii) a metabolic disorder; and/or (ix) a cancer.
222. The use of one or more CIICs or higher order CIIC complexes (e.g., a duplexes) of aspects 1-171 in the treatment of T1D or celiac disease.
223. The use of one or more CIICs or higher order CIIC complexes (e.g., a duplex) of aspects 1-171, for the preparation of a medicament for use in the treatment of: (i) an autoimmune disease other than, or in addition to, celiac disease and/or T1D; (ii) allergy (e.g., an allergic reaction to a specific antigen); (iii) metabolic diseases;
   (iv) infections by viral agents; (v) infections by non-viral agents; (vi) GVHD; (vii) HVGD; (viii) metabolic disorders; and/or (ix) cancers.
224. The use of one or more CIICs or higher order CIIC complexes (e.g., a duplexes) of aspects 1-171, for the preparation of a medicament for use in the treatment of T1D or celiac disease.
225. A CIIC or higher order CIIC complex (e.g., a duplex) thereof according to any of aspects 1-100, further comprising a MAS or a polypeptide sequence to which a lipid group (e.g., prenyl, phospholipid farnesyl group etc.) has been added. (see, e.g., FIG. 1, structures J-M.) 226. A CIIC or higher order CIIC complex (e.g., a duplex) thereof according to any of aspects 101-169, further comprising a MAS or a polypeptide sequence to which a lipid group (e.g., prenyl, phospholipid farnesyl group etc.) has been added.
227. One or more nucleic acids or recombinant expression vectors comprising a sequence encoding a CIIC or higher order CIIC complex (e.g., a duplex) thereof according to claim 225 or 226.
228. A cell, liposome, or vesicle comprising a CIIC or higher order CIIC complex (e.g., a duplex) of any of aspects 225 or 226, wherein the CIIC or higher order CIIC complex (e.g., a duplex) is associated with the cell, liposome, or vesicle by interaction with the MAS or lipid group.
229. The cell of aspect 228, wherein the cell is an artificial antigen presenting cell.
230. The cell of aspect 229, wherein the artificial antigen presenting cell is an engineered erythroid cell or enucleated cell (such as an enucleated erythroid cell or platelet).
231. A pharmaceutical composition comprising one or more CIICs or higher order CIIC complexes (e.g., a duplex) of any of aspects 225-226 or one or more cells, liposomes, or vesicles of any of aspects 228 to 230.
232. A pharmaceutical composition comprising one or more nucleic acids or recombinant expression vectors of aspect 227.
233. A method of treatment or prophylaxis of a patient or subject having a disease or condition comprising:
   (i) administering to a patient/subject (e.g., a patient in need thereof) an effective amount of one or more CIICs or higher order CIIC complexes (e.g., a duplex) of any of aspects 225-226, or one or more cells, liposomes, or vesicles of any of aspects 228 to 230 or a pharmaceutical composition of aspect 231;
   (ii) administering to a patient/subject (e.g., a patient in need thereof) an effective amount of one or more nucleic acids encoding a one or more CIICs or higher order CIIC complexes (e.g., a duplex) aspect 203 or a pharmaceutical composition according to aspect 232;
   (iii) contacting a cell or tissue of a patient or subject, either in vitro or in vivo, with one or more CIICs or higher order CIIC complexes (e.g., a duplex) of any of aspects 228 to 230, one or more cells, liposomes, or vesicles of any of aspects 228 to 230, or a pharmaceutical composition of aspect 231, and administering the cell or tissue of the patient or subject subjected to the contacting, or progeny thereof, to the patient/subject; or
   (iv) contacting a cell or tissue of a patient or subject, either in vitro or in vivo, with one or more nucleic acids encoding one or more CIICs or higher order CIIC complexes (e.g., a duplex) of aspect 227, and administering the cell or tissue of the patient or subject, or progeny thereof, to the patient/subject.
234. The method of claim 233, wherein said disease or condition is selected from condition cancer, allergy, autoimmunity, T1D, celiac disease, GVHD, HGVD, infection, a metabolic disorder such as T2D or NAFLD, or combination thereof.
235.A CIIC protein or duplex CIIC protein comprised of two monomers of the CIIC protein, the CIIC having the structure:
   (i) peptide epitope—peptide linker (e.g., (G₄S)₃)—DQB1β1 domain (with a Cys substitution in one of N-terminal eight aas )—DQB1β2 domain—peptide linker (e.g., (G₄S)₅)—DQA1α1 domain (with a Cys substitution in one of the C-terminal six aas 1-8)—DQA1α2 domain—peptide linker (e.g., GGSAAAGG, SEQ ID NO:249)—human IgG1 Fc domain optionally having a LALA substitutions, wherein each monomer has a body disulfide bond between the Cys substitution in one of N-terminal eight aas of the β1 domain and the Cys substitution in one of the C-terminal six aas of the α1 domain; or the structure
   (ii) peptide epitope—peptide linker (e.g., (G₄S)₃)—DRB1β1 domain (with a Cys substitution in one of N-terminal eight aas )—DRB1β2 domain—peptide linker (e.g., (G₄S)₅)— DRA1α1 domain (with a Cys substitution in one of the C-terminal six aas 1-8)—DRA1α2 domain—peptide linker (e.g., GGSAAAGG, SEQ ID NO:249)—human IgG1 Fc domain optionally having a LALA substitutions, wherein each monomer has a body disulfide bond between the Cys substitution in one of N-terminal eight aas of the β1 domain and the Cys substitution in one of the C-terminal six aas of the α1 domain; or the structure
   (iii) peptide epitope—peptide linker (e.g., (G₄S)₃)—DPB1β1 domain (with a Cys substitution in one of N-terminal eight aas )—DPB1β2 domain—peptide linker (e.g., (G₄S)₅)— DPA1α1 domain (with a Cys substitution in one of the C-terminal six aas 1-8)—DPA1α2 domain—peptide linker (e.g., GGSAAAGG, SEQ ID NO:249)—human IgG1 Fc domain optionally having a LALA substitutions; wherein each monomer has a body disulfide bond between the Cys substitution in one of N-terminal eight aas of the β1 domain and the Cys substitution in one of the C-terminal six aas of the α1 domain; and wherein the peptide epitope is a peptide epitope other than an epitope of a TiD-associated antigen or an epitope of a celiac associated antigen.
236. A CIIC protein or duplex CIIC protein comprised of two monomers of the CIIC protein, wherein the protein comprises an aa sequence having at least 90%, or at least 95%, sequence identity to the aa sequence:

(SEQ ID NO: 154)
GGGGSGGGGSGGGGSRDSPC*DFVYQFKGMCYFTNGTERVRLVSRSIYNREEIVRFDSDVGEFRAVTLLGLPA
AEYWNSQKDILERKRAAVDRVCRHNYQLELRTTLQRRVEPTVTISPSRTEALNHHNLLVCSVTDFYPAQIKVRW
FRNDQEETAGVVSTPLIRNGDWTFQILVMLEMTPQRGDVYTCHVEHPSLQSPITVEWRAQSESAQSKGGGGSG
GGGSGGGGSGGGGSGGGGSEDIVADHVASYGVNLYQSYGPSGQYTHEFDGDEQFYVDLGRKETVWKLPVLH
QFRFDPQFALTNIAVLKHNLNILIKRSNSTC*ATNEVPEVTVFSKSPVTLGQPNILICLVDNIFPPVVNITWLSNGHS
VTEGVSETSFLSKSDHSFFKISYLTLLPSAEESYDCKVEHWGLDKPLLKHWEPEIPAPMSELTEGGSAAAGGDK
THTCPPCPAPEAAGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEALHNHYTQKSLS
LSPG,
or
the aa sequence
(SEQ ID NO: 156)
GGGGSGGGGSGGGGSGDTRC*RFLEQVKHECHFFNGTERVRFLDRYFYHQEEYVRFDSDVGEYRAVTELGR
PDAEYWNSQKDLLEQKRAAVDTYCRHNYGVGESFTVQRRVYPEVTVYPAKTQPLQHHNLLVCSVNGFYPASIE
VRWFRNGQEEKTGVVSTGLIQNGDWTFQTLVMLETVPRSGEVYTCQVEHPSLTSPLTVEWRARSESAQSKMG
GGGSGGGGSGGGGSGGGGSGGGGSIKEEHVIIQAEFYLNPDQSGEFMFDFDGDEIFHVDMAKKETVWRLEEF
GRFASFEAQGALANIAVDKANLEIMTKRSNYTC*ITNVPPEVTVLTNSPVELREPNVLICFIDKFTPPVVNVTWLRN
GKPVTTGVSETVFLPREDHLFRKFHYLPFLPSTEDVYDCRVEHWGLDEPLLKHWEFDAPSPLPETGGSAAAGG
DKTHTCPPCPAPEAAGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMHEALHNHYTQKS
LSLSPG;
or
the sequence
(SEQ ID NO: 370)
GGGGSGGGGSGGGGSPC*NYLFQGRQECYAFNGTQRFLERYIYNREEFARFDSDVGEFRAVTELGRPAAEYW
NSQKDILEEKRAVPDRMCRHNYELGGPMTLQRRVQPRVNVSPSKKGPLQHHNLLVCHVTDFYPGSIQVRWFLN
GQEETAGVVSTNLIRNGDWTFQILVMLEMTPQQGDVYTCQVEHTSLDSPVTVEWKAQGGGGSGGGGSGGGG
SGGGGSGGGGSIKADHVSTYAAFVQTHRPTGEFMFEFDEDEMFYVDL[D]KKETVW(H)LEEFGQAFSFEAQGG
LANIAILNNNLN(T)LIQRSNHT(C*)ATNDPPEVTVFPKEPVELGQPNTLICHIDKFFPPVLNVTWLCNGELVTEGVA
ESLFLPRTDYSFHKFHYLTFVPSAEDFYDCRVEHWGLDQPLLKHWEAQGGSAAAGGDKTHTCPPCPAPEAAG
GPSVFLFPPKPKDTLMISRTPEVTCVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYTQKSLSLSPG;

wherein the cysteine residues indicated by an asterisk (C*) form an intrachain disulfide bond; wherein the protein is optionally in the form of a duplex; and
wherein the protein comprises a peptide epitope (e.g., an epitope peptide other than a peptide epitope of a T1D-celiac-associated antigen) bound to its N-terminus as part of the protein sequence.
237. A protein comprising an aa sequence having at least 90%, or at least 95%, sequence identity to the aa sequence:

(SEQ ID NO: 371)
GGGGSGGGGSGGGGSRDSPC*DFVYQFKGMCYFTNGTERVRLVSRSIYNREEIVRFDSDVGEFRAVTLLGLPA
AEYWNSQKDILERKRAAVDRVCRHNYQLELRTTLQRRVEPTVTISPSRTEALNHHNLLVCSVTDFYPAQIKVRW
FRNDQEETAGVVSTPLIRNGDWTFQILVMLEMTPQRGDVYTCHVEHPSLQSPITVEWRAQSESAQSKGGGGSG
GGGSGGGGSGGGGSGGGGSEDIVADHVASYGVNLYQSYGPSGQYTHEFDGDEQFYVDLGRKETVWKLPVLH
QFRFDPQFALTNIAVLKHNLNILIKRSNSTC*ATNEVPEVTVFSKSPVTLGQPNILICLVDNIFPPVVNITWLSNGHS
VTEGVSETSFLSKSDHSFFKISYLTLLPSAEESYDCKVEHWGLDKPLLKHWEPEIPAPMSELTEGGSAAAGGDK
THTCPPCPAPEAAGGPSVFLFPPKPKDTLMISRTPEVTCVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEALHNHYTQKSLS
LSPG,
or
the aa sequence
(SEQ ID NO: 156)
GGGGSGGGGSGGGGSGDTRC*RFLEQVKHECHFFNGTERVRFLDRYFYHQEEYVRFDSDVGEYRAVTELGR
PDAEYWNSQKDLLEQKRAAVDTYCRHNYGVGESFTVQRRVYPEVTVYPAKTQPLQHHNLLVCSVNGFYPASIE
VRWFRNGQEEKTGVVSTGLIQNGDWTFQTLVMLETVPRSGEVYTCQVEHPSLTSPLTVEWRARSESAQSKMG
GGGSGGGGSGGGGSGGGGSGGGGSIKEEHVIIQAEFYLNPDQSGEFMFDFDGDEIFHVDMAKKETVWRLEEF
GRFASFEAQGALANIAVDKANLEIMTKRSNYTC*ITNVPPEVTVLTNSPVELREPNVLICFIDKFTPPVVNVTWLRN
GKPVTTGVSETVFLPREDHLFRKFHYLPFLPSTEDVYDCRVEHWGLDEPLLKHWEFDAPSPLPETGGSAAAGG
DKTHTCPPCPAPEAAGGPSVFLFPPKPKDTLMISRTPEVTCVVDVSHEDPEVKFNWYVDGVEVHNAKTKPRE
EQYNSTYRVVSVLTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMHEALHNHYTQKS
LSLSPG;

wherein the cysteine residues indicated by an asterisk (C*) form an intrachain disulfide bond; wherein the protein is optionally in the form of a duplex; and wherein the protein comprises a T1D-epitope or celiac-epitope bound to its N-terminus as part of the protein sequence.
238. The protein of aspect 237, comprising an aa sequence having at least 97%, or at least 98%, sequence identity to the aa sequence of SEQ ID NO:371 or SEQ ID NO:156.
239. The protein of aspect 237, comprising an aa sequence having at least 90% or least 95% sequence identity to the aa sequence of SEQ ID NO:371 or SEQ ID NO:156, wherein the percent identity is calculated excluding the linkers sequences comprising one or more repeats of the aa sequence GGGGS (SEQ ID NO. 237).
240. The protein of aspect 237, comprising an aa sequence having at least 97%, or at least 98%, sequence identity to the aa sequence of SEQ ID NONO:371 or SEQ ID NO:156, wherein the percent identity is calculated excluding the linkers sequences comprising one or more repeats of the aa sequence GGGGS (SEQ ID NO. 237).
241. The protein of any of aspects 237-240, comprising a body disulfide bond.
242. The protein of aspect 237, wherein the T1D-epitope or celiac-epitope binds to MHC molecules of the DR4 serotype, HLA DQ2.5 serotype, or molecules of the DQ8.1 serotype.
243.A method treating a patient or subject (e.g., a patient or subject in need thereof), the method comprising administering the protein or a pharmaceutical composition comprising a protein, of any of aspects 236-242.
244. The method of aspect 243, wherein the patient suffers from a cancer, an allergy, an autoimmune disease other than or in addition to T1D and/or celiac disease, GVHD, HGVD, an infection, or a metabolic disorder.
245. The method of aspect 243, wherein the patient suffers from T1D or celiac disease.
246.A method of producing cells expressing a CIIC or higher order map (e.g., duplex CIIC), the method comprising introducing one or more nucleic acid molecules or expression vectors according to aspect 173 or 174, into the cells in vitro, selecting for cells that produce the CIIC or duplex CIIC, and optionally selecting for cells comprising all or part of the one or more nucleic acids either unintegrated or integrated into at least one cellular chromosome.
247. The method of aspect 246, wherein the cell is a cell of a mammalian cell line selected from the HeLa cells, CHO cells, 293 cells, Vero cells, NIH 3T3 cells, Huh-7 cells, BHK cells, PC12, COS cells, COS-7 cells, RAT1 cells, mouse L cells, human embryonic kidney (HEK) cells, and HLHepG2 cells.
248. One or more cells transiently or stably expressing a CIIC or duplex CIIC prepared by the method of aspect 246 or 247.
249. The cells of aspect 248, wherein the cells express from about 25 to about 350 (e.g., 20-50, 50-100, 100-200, 200-300, 300-350) mg/I or more of the CIIC or duplex CIIC without a substantial reduction (e.g., less than a 5%, 10%, or 15% reduction) in viability relative to otherwise identical cells not expressing the CIIC or duplex CIIC.
250. A method of selectively delivering one or more MODs and/or variant MODs to a cell or tissue of a patient or subject having or suspected of having T1D or celiac disease, the method comprising administering to a patient/subject having or suspected of having T1D or celiac disease an effective amount of one or more CIICs or higher order CIICS (e.g., duplex CIICs) of any of aspects 1-171.
251. A polypeptide or protein comprising an aa sequence that comprises in the N-terminal to C-terminal direction:
   (i) optionally an L1 aa linker sequence comprising a cysteine,
   (ii) an HLA Class II β chain polypeptide sequence (comprising e.g., a β1 and β2 domain sequence);
   (iii) optional an L2 aa linker sequence, and
   (iv) an HLA Class II α chain polypeptide sequence (comprising e.g., an α1 and α2 domain sequence); wherein
   (A) the polypeptide or protein comprises either
      (a) a body disulfide bond between a cysteine in the β1 domain (e.g., a cysteine substituted for one of the N-terminal 8 aas of the β1 domain) and the α1 domain (e.g., a cysteine substituted for one of the C-terminal 11 aas of the α1 domain), or
      (b) a linker disulfide bond between a cysteine in the L1 linker and a cysteine in the α1 domain (e.g., a cysteine substituted for one of the C-terminal 11 aas of the α1 domain), wherein when the L1 aa linker comprises a cysteine (e.g., in its N-terminal 5 aas) then the polypeptide or protein comprises a linker disulfide bond; and
   (B) optionally, wherein when the polypeptide or protein comprises a cysteine at aa 43 through aa 48 of the α chain polypeptide sequence (α1 and α2 domain sequence), it is substituted by an aa other than cysteine (e.g., a C47S, C47R or C47K substitution in DQA*05:01).
252. The polypeptide or protein of aspect 251, comprising an L2 aa linker from 1-50 aas in length.
253. The polypeptide or protein of aspect 251 or 252, comprising an L2 aa linker from 5-40 aas in length.
254. The polypeptide or protein of aspect 252 or 253, wherein the L2 aa linker comprises glycine, serine, or alanine.
255. The polypeptide or protein of any of aspects 252 to 254, wherein the total number of glycine, serine, and alanine residues in the L2 aa linker is greater than 50% or greater than 75% of the aa residues in the L2 linker.
256. The polypeptide or protein of any of aspects 251 to 255, wherein:
   the HLA Class II β chain polypeptide sequence comprises a DQB (e.g., DQB1 or DQB2) β1 and β2 domain sequence; and
   the HLA Class II α chain polypeptide sequence comprises a DQA (e.g., DQA1 or DQA2) α1 and α2 domain sequence.
257. The polypeptide or protein of any of aspects 251 to 256, wherein the HLA Class II β chain polypeptide sequence has at least 90% or at least 95% aa sequence identity to all or at least 170 contiguous aas of a DQB p1 and β2 domain sequence of DQB1*02:01, DQB1*02:02, DQB1*03:01, DQB1*03:02, DQB1*03:03, DQB1*03:04, DQB1*04:01, DQB1*04:02, DQB1*05:01, DQB1*06:01, DQB1*06:02, DQB2 isoform 1 or DQB2 isoform 2; and/or
   the HLA Class II α chain polypeptide sequence has at least 90% or at least 95% aa sequence identity to all or least 165 contiguous aas of the DQA α1 and α2 domain sequence of DQA1*01:01, DQA1*01:02, DQA1*01:03, DQA1*01:04, DQA1*02:01, DQA1*03:01, DQA1*03:02, DQA1*04:01, DQA1*05:01, DQA1*05:05, DQA1*06:01, or DQA2*01:01.
258. The polypeptide or protein of any of aspects 251 to 256, wherein the HLA Class II β chain polypeptide sequence has at least 90% or at least 95% aa sequence identity to all or at least 170 contiguous aas of a DQB p1 or β2 domain sequence of DQB1*02:01, DQB1*02:02, DQB1*03:01, DQB1*03:02, DQB1*04:01, DQB1*05:01, DQB1*06:01, DQB1*06:02, DQB2 isoform 1 or DQB2 isoform 2, and
   the HLA Class II α chain polypeptide sequence has at least 90% or at least 95% aa sequence identity to all or at least 165 contiguous aas of the DQA α1 or α2 domain sequence of DQA1*01:01, DQA1*01:02, DQA1*01:03, DQA1*01:04, DQA1*02:01, DQA1*03:01, DQA1*03:02, DQA1*04:01, DQA1*05:01, DQA1*05:05, DQA1*06:01, or DQA2*01:01.
259. The polypeptide or protein of aspect 257 or 258, wherein the sequences to which the DQB β1 and β2 domain sequence and the DQA α1 and α2 domain sequence have at least 90% or at least 95% aa sequence identity are a DQB and DQA allele pair selected from:
   (i) DQB1*02:01 and DQA1*05:01 (DQ2.5);
   (ii) DQB1*02:02 and DQA1*02:01 (DQ2.2);
   (iii) DQB1*03:02 and DQA1*03:01 (DQ8.1);
   (iv) DQB1*04:02 and DQA1*04:01 (DQ4.2);
   (v) DQB1*04:01 and DQA1*03:01 (DQ4.3a)
   (vi) DQB1*04:02 and DQA1*03:01 (DQ4.3b);
   (vii) DQB1*05:01 and DQA1*01:01; or
   (viii) DQB1*06:02 and DQA1*01:02 (DQ6.2).
260. The polypeptide or protein of aspect 259, wherein the sequences to which the DQB β1 and β2 domain sequence and the DQA α1 and α2 domain sequence have at least 90% or at least 95% aa sequence identity are a DQB and DQA allele pair selected from:
   (i) hDQ2.5-beta1-2(E5C)-(G₄S)₅-alpha1-2(C47K,A83C (DQ2.5); or
   (ii) DQB1*03:02 and DQA1*03:01 (DQ8.1).
261. The polypeptide or protein of any of aspects 256 to 260, comprising a body disulfide bond formed between the N-terminal portion (e.g., the N-terminal 8 amino acids) of the DQB1 or DQB2 β1 domain and the C terminal portion (e.g., the C-terminal 6 amino acids) of the DQA α1 domain sequence.
262. The polypeptide or protein of aspect 261, wherein the body disulfide bond is formed between a cysteine substituted at position 4, 5, 6, or 7 of the DQB1 or DQB2 β1 domain and a cysteine substituted at position 83, 84, or 85 of the DQA1 or DQA2 α1 domain sequence.
263. The polypeptide or protein of aspect 262, comprising a body disulfide bond formed between a cysteine substituted at position 5 of the DQB1 (e.g., E5C) or DQB2 (e.g., K5C) β1 domain and a cysteine substituted at position 82, 83, 84 or 85 (e.g., A83C of DQA1*05:01) of the DQA1 or DQA2 α1 domain sequence.
264. The polypeptide or protein of any of aspects 256 to 260, comprising a linker disulfide bond formed between a cysteine in the L1 linker sequence and a cysteine substitution at position 76, 77, 78, or 79 of the DQA1 or DQA2 α1 domain sequence.
265. The polypeptide or protein of aspect 264, wherein the substitution at position 76, 77, 78, or 79 is a K77C or K78C substitution in DQA1 or an R78C substitution in DQA2.
266. The polypeptide or protein of any of aspects 256 to 265, comprising a substitution at any one or more of positions 40, 52, 74 or 75 of the DQA1 or DQA2 α1 domain.
267. The polypeptide or protein of any of aspects 256 to 266, wherein position 40 of the DQA1 α1 domain is an E (e.g., a G40E substitution), position 52 is an H (e.g., a R52H or S52H substitution), and/or position 74 or 75 is an I (e.g., a S741 substitution in DQA1*05:01 or a F751 substitution in DQA2*01:01).
268. The polypeptide or protein of any of aspects 256 to 267, wherein:
   (i) aa position 47 of the DQA (e.g., DQA1 or DQA2) α1 domain sequence is an aa other than cysteine; or
   (ii) position 47 is a serine, lysine, or arginine, or is substituted by a serine, lysine, or arginine.
269. The polypeptide or protein of aspects 251 to 259, comprising an aa sequence:
   (A) having at least 90% or at least 95% aa sequence identity to the aa sequence of hDQ2.5-beta1-2(E5C)-(G₄S)₅-alpha1-2(C47K, A83C (DQ2.5)) RDSPC*DFVYQFKGMCYFTNGTERVRLVSRSIYNREEIVRFDSDVGEFRAVTLLGLPAAEYWNSQKDILERKR AAVDRVCRHNYQLELRTTLQRRVEPTVTISPSRTEALNHHNLLVCSVTDFYPAQIKVRWFRNDQEETAGVVST PLIRNGDWTFQILVMLEMTPQRGDVYTCHVEHPSLQSPITVEWRAQSESAQSK-[L2]-EDIVADHVASYGVNLYQSYGPSGQYTHEFDGDEQFYVDLGRKETVWKLPVLRQFRFDPQFALTNIAVLKHNL N(S)LIKRSNSTC*ATNEVPEVTVFSKSPVTLGQPNILICLVDNIFPPVVNITWLSNGHSVTEGVSETSFLSKSDHS FFKISYLTLLPSAEESYDCKVEHWGLDKPLLKHWEPEIPAPMSELTE (SEQ ID NO:499); wherein the cysteine residues denoted by “C*” form a body disulfide; and
      wherein the intervening linker, L2, is not considered when calculating aa sequence identity;
   (B) having in the N-terminal to C terminal direction the aa sequence RDSPC*DFVYQFKGMCYFTNGTERVRLVSRSIYNREEIVRFDSDVGEFRAVTLLGLPAAEYWNSQKDILERKR AAVDRVCRHNYQLELRTTLQRRVEPTVTISPSRTEALNHHNLLVCSVTDFYPAQIKVRWFRNDQEETAGVVST PLIRNGDWTFQILVMLEMTPQRGDVYTCHVEHPSLQSPITVEW (SEQ ID NO:324), or an aa sequence having at least 90% or at least 95% sequence identity to SEQ ID NO:324 and the aa sequence EDIVADHVASYGVNLYQSYGPSGQYTHEFDGDEQFYVDLGRKETVWKLPVLHQFRFDPQFALTNIAVLKHNL NILIKRSNSTC*ATNEVPEVTVFSKSPVTLGQPNILICLVDNIFPPVVNITWLSNGHSVTEGVSETSFLSKSDHSFF KISYLTLLPSAEESYDCKVEHWGLDKPLLKHWEPEIPAPMSELTE (SEQ ID NO:325) or an aa sequence having at least 90% or at least 95% sequence identity to SEQ ID NO:325; wherein the cysteine residues denoted by “C*” form a body disulfide;
   (C) having at least 90% or at least 95% aa sequence identity to human DQ8.1 sequences DQB1*03:02 beta1-2 (E5C) and DQA1*03:01 (A83C) RDSPC*DFVYQFKGMCYFTNGTERVRLVTRYIYNREEYARFDSDVGVYRAVTPLGPPAAEYWNSQKEVLERT RAELDTVCRHNYQLELRTTLQRRVEPTVTISPSRTEALNHHNLLVCSVTDFYPAQIKVRWFRNDQEETTGVVS TPLIRNGDWTFQILVMLEMTPQRGDVYTCHVEHPSLQNPIIVEW-[L2]-EDIVADHVASYGVNLYQSYGPSGQYSHEFDGDEEFYVDLERKETVWQLPLFRRFRRFDPQFALTNIAVLKHNL NIVIKRSNSTC*ATNEVPEVTVFSKSPVTLGQPNTLICLVDNIFPPVVNITWLSNGHSVTEGVSETSFLSKSDHSF FKISYLTFLPSDDEIYDCKVEHWGLDEPLLKHW (SEQ ID NO:500); wherein the cysteine residues denoted by “C*” form a body disulfide; and
      wherein the intervening linker, L2, is not considered when calculating aa sequence identity; or
(D) having in the N-terminal to C terminal direction
   1) an aa sequence comprising aas 1-188 of SEQ ID NO:92 with a P4C, E5C, D6C or F7C substitution, or an aa sequence having at least 90% or at least 95% sequence identity to SEQ ID NO:92 with a P4C, E5C, D6C or F7C substitution; and
   2) an aa sequence comprising aas 1-181 of SEQ ID NO:92 with a T82C, A83C, or Δ84C substitution or an aa sequence having at least 90% or at least 95% sequence identity to SEQ ID NO:92 with a T82C, A83C, or A84C,
   wherein a cysteine of a P4C, E5C, D6C or F7C substitution forms a body disulfide bond with a cysteine of a T82C, A83C, or Δ84C substitution.
270. The polypeptide or protein of any of aspects 256 to 268, wherein the DQA and DQB sequences are an allele pair selected from:
   HLA-DQA1*05:01 and DQB1*02:01 (DQ2.5), or
   DQB1*03:02 and DQA1*03:01 (DQ8.1)
271. The polypeptide or protein of any of aspects 256 to 268, wherein the DQA and DQB sequences are an allele pair selected from:
   DQB1*02:02 and DQA1*02:01 (DQ2.2),
   DQB1*05:01 and DQA1*01:01, or
   DQB1*06:02 and DQA1*01:02 (DQ6.2); and
   the L2 linker optionally comprises from 3 to 8 repeats of GGGGS.
272. The polypeptide or protein of any of aspects 256 to 268, wherein the DQA and DQB sequences are an allele pair selected from:
   DQB1*04:01 and DQA1*03:01 (DQ4.3a),
   DQB1*04:02 and DQA1*03:01 (DQ4.3b), or
   DQB1*04:02 and DQA1*04:01 (DQ4.2); and
   the L2 linker optionally comprises from 3 to 8 repeats of GGGGS.
273. The polypeptide or protein of any of aspects 251 to 255, wherein:
   the HLA Class II β chain polypeptide sequence comprises a DPB (e.g., DPB1) β1 and β2 domain sequence; and
   the HLA Class II α chain polypeptide sequence comprises a DPA (e.g., DPA1) α1 and α2 domain sequence.
274. The polypeptide or protein of any of aspects 251 to 255 and 273, wherein:
   the HLA Class II β chain polypeptide sequence has at least 90% or at least 95% aa sequence identity to all or at least 165 contiguous aas of a DPB β1 and β2 domain sequence of DPB1*01:01, DPB1*02:01, DPB1*03:01, DPB1*04:01, DPB1*06:01, DPB1*09:01, DPB1*11:01, DPB1*13:01, DPB1*35:01, DPB1*71:01, DPB1*104:01, or DPB1*141:01, and
   the HLA Class II α chain polypeptide sequence has at least 90% or at least 95% aa sequence identity to all or at least 165 contiguous aas of the DPA α1 and α2 domain sequences of DPA1*01:03 or DPA1*02:01.
275. The polypeptide or protein of any of aspects 251 to 255 and 273, wherein:
   the HLA Class 11 chain polypeptide sequence has at least 90% or at least 95% aa sequence identity to all or at least 165 contiguous aas of a DPB β1 or β2 domain sequence of DPB1*01:01, DPB1*03:01, DPB1*09:01, DPB1*13:01, or DPB1*35:01, and
   the HLA Class II α chain polypeptide sequence has at least 90% or at least 95% aa sequence identity to all or at least 165 contiguous aas of the DPA α1 or α2 domain sequences of DPA1*01:03 or DPA1*02:01.
276. The polypeptide or protein of any of aspects 273 to 275, comprising a body disulfide bond formed between an N-terminal portion (e.g., the N-terminal 8 amino acids) of the DPB β1 domain and a C-terminal portion (e.g., the C-terminal 6 amino acids) of the DPA α1 domain.
277. The polypeptide or protein of aspect 276, wherein the body disulfide bond is formed between a cysteine substituted at position 4, 5, 6, or 7, of the DPB β1 domain and a cysteine substituted at position 83, 84, or 85 of the DPA α1 domain.
278. The polypeptide or protein of aspect 277 comprising a body disulfide bond formed between a cysteine substituted at position 4, 5 6, 7, or 8 (e.g., a P4C, ESC, N6C or Y7C) of the DPB β1 domain and a cysteine substituted at position 84 (Q84C) of the DPA α1 domain.
279. The polypeptide or protein of any of aspects 273 to 278, comprising a linker disulfide bond formed between a cysteine in the L1 linker sequence and a cysteine at position 77, 78, or 79 of the DPA α1 domain.
280. The polypeptide or protein of any of aspects 273 to 279, comprising a substitution at any one or more of positions 40, 47, 52 or 75 of the DPA α1 domain.
281. The polypeptide or protein of any of aspects 273 to 280, wherein position 47 of the DPA α1 domain is a D or an E (e.g., a D40E substitution), and/or position 52 is an H (e.g., a G52H substitution), and/or position 75 is an Ile (e.g., a T401 substitution).
282. The polypeptide or protein of any of aspects 273 to 281, wherein:
   (i) aa position 47 of the DPA α1 domain sequence is an aa other than cysteine; or
   (ii) aa position 47 of the DPA α1 domain sequence is a serine, lysine, or arginine, or is substituted by a serine, lysine, or arginine.
283. The polypeptide or protein of any of aspects 273 to 282, wherein the DPB and DPBA aa sequences are DPB1*03:01 and DPA1*01:03.
284. The polypeptide or protein of any of aspects 251 to 255, wherein:
   the HLA Class II p3 chain polypeptide sequence comprises a DRB (e.g., DRB1, DRB3, DRB4 or DRB5) β1 and β2 domain sequence; and
   the HLA Class II α chain polypeptide sequence comprises a DRA α1 and α2 domain sequence (e.g., DRA*01:01 or DRA*01:02).
285. The polypeptide or protein of any of aspects 251 to 255 or 284, wherein:
   the HLA Class II β chain polypeptide sequence has at least 90% or at least 95% aa sequence identity to all or at least 170 contiguous aas of a DRB β1 and β2 domain sequence of DRB1*01:01, DRB1*01:02, DRB1*01:03, DRB1*03:01, DRB1*03:02, DRB1*03:04, DRB1*04:01, DRB1*04:02, DRB1*04:03, DRB1*04:04, DRB1*04:05, DRB1*04:06, DRB1*04:08, DRB1*07:01, DRB1*08:01, DRB1*08:02, DRB1*08:03, DRB1*09:01, DRB1*10:01, DRB1*11:01, DRB1*11:03, DRB1*11:04, DRB1*12:01, DRB1*13:01, DRB1*13:03, DRB1*14:01, DRB1*14:02, DRB1*14:05, DRB1*14:06, DRB1*15:01, DRB1*15:02, DRB1*15:03, DRB1*15:04, DRB1*15:05, DRB1*15:06, DRB1*15:07, DRB1*16:01, DRB3*01:01, DRB3*02:01, DRB3*03:01, DRB4*01:01, DRB4*01:03, or DRB5*01:01; and the HLA Class II α chain polypeptide sequence has at least 90% or at least 95%% aa sequence identity to at least 165 contiguous aas of the DRA α1 and α2 domain sequence of DRA1*01:01 or DRA*01:02.
286. The polypeptide or protein of any of aspects 251 to 255 or 284, wherein
   the HLA Class II β chain polypeptide sequence has at least 90% or at least 95% aa sequence identity to at least 165 contiguous aas of a DRB β1 and β2 domain sequence of DRB1*03:02, DRB1*03:04, DRB1*04:01, DRB1*04:02, DRB1*04:03, DRB1*04:04, DRB1*04:05, DRB3*01:01, DRB3*02:01, DRB3*03:01, DRB4*01:01, or DRB5*01:01, and/or
   the HLA Class II α chain polypeptide sequence has at least 90% or at least 95% aa sequence identity to at least 165 contiguous aas of the DRA α1 and α2 domain sequence of DRA1*01:01 or DRA*01:02.
287. The polypeptide or protein of any of aspects 284 to 286, comprising a body disulfide bond formed between an N-terminal portion (e.g., the N-terminal 8 amino acids) of the DRB β1 domain and a C-terminal portion (e.g., the C-terminal 6 amino acids) of the DRA α1 domain.
288. The polypeptide or protein of aspect 287, wherein the body disulfide bond is formed between a cysteine substituted at position 4, 5, 6, or 7, of the DRB β1 domain and a cysteine substituted at position 80, 81, or 82 of the DRA1 α1 domain.
289. The polypeptide or protein of aspect 288, comprising a body disulfide bond formed between a cysteine substituted at position 5 (e.g., a P5C) of the DRB β1 domain and a cysteine substituted at position 81 (P81C) of the DRA α1 domain.
290. The polypeptide or protein of any of aspects 284-289, comprising a linker disulfide bond formed between a cysteine in the L1 linker sequence and a cysteine at position 74, 75, or 76 of the DRA α1 domain.
291. The polypeptide or protein of any of aspects 284-290, comprising a substitution at any one or more of positions 37, 49 or 72 of the DRA α1 domain.
292. The polypeptide or protein of any of aspects 284-291, wherein position 37 of the DRA α1 domain is an E (e.g., an Δ37E substitution), and/or position 49 is an H (e.g., a G49H substitution), and/or position 72 is an I (lie).
293. The polypeptide or protein of any of aspects 284-292, wherein
   (i) aa position 44 of the DRA α1 domain sequence is an aa other than cysteine; or
   (ii) aa position 44 of the DRA α1 domain sequence is a serine, lysine, or arginine, or is substituted by a serine, lysine, or arginine.
294. The polypeptide or protein of any of aspects 284-292, wherein the DRB and DRA sequences are allele pair selected from:
   DRB1*03:01 and DRA1*01:02, or
   DRB1*04:01 and DRA1*01:02; and
the L2 linker optionally comprises from 3 to 8 repeats of GGGGS 295. The polypeptide or protein of any of aspects 284-292, wherein the DRB and DRA sequences are allele pair selected from:
   DRB1*1101 and DRA1*01:02, or
   DRB1*1501 and DRA1*01:02; and
the L2 linker optionally comprises from 3 to 8 repeats of GGGGS.
296. The polypeptide or protein of any of aspects 284-292, wherein the DRB and DRA sequences are an allele pair selected from:
   DRB3*01:01 and DRA1*01:02, or
   DRB4*01:01 and DRA1*01:02; and
the L2 linker optionally comprises from 3 to 8 repeats of GGGGS.
297. The polypeptide or protein of any of aspects 251-296, further comprising a peptide epitope that is either: (i) part of the polypeptide or protein (e.g., located N-terminal to the HLA Class II β chain polypeptide sequence and optionally attached by an L1 linker; or (ii) non-covalently associated (e.g., dissociable bound) to the polypeptide or protein.
298. The polypeptide or protein of any of aspects 251-297, further comprising a MOD sequence.
299. A method of treatment comprising administering to a subject an amount of a protein or polypeptide of any of aspects 251-298, wherein the treatment is for an autoimmune disease, cancer, GVHD, HVGD, an allergy, or an infection.
300. The use of a polypeptide or protein of any of aspects 251-298 for the preparation of a medicament for the treatment of an autoimmune disease, cancer, GVHD, HVGD, an allergy, or an infection.
301. A polypeptide or protein of any of aspects 251-298 for the treatment of an autoimmune disease, cancer, GVHD, HVGD, an allergy, or an infection.
302. A nucleic acid or a recombinant vector encoding the polypeptide or protein of any of aspects 251-298.

A skilled artisan will understand that components such as linkers, scaffolds, epitopes, additional polypeptides, payloads, and the like that are suitable for CIICs may be utilized in the polypeptides or proteins of immediately preceding aspects 251-302.

Throughout the aspects amino acid sequence identities of at least 90% or at least 95% can be increased to at least 96% or at least 98% aa sequence identity. Similarly, amino acid sequence identities of at least 90% can be increased to at least 96% or at least 98% aa sequence identity.

### VIII. Examples

### Example 1. Body Disulfide Incorporation

In order to determine the effect of body disulfide bonds on CIICs, five soluble constructs comprising HLA DQ 2.5 sequences (HLA DQB1*02:01 β1 and p2domains and DQA1:05:01 α1 and α2 domains) and Ig Fc scaffolds were prepared (see constructs 3832-3836 in FIG. 18). The peptide epitope utilized was an anchor-modified variant of an MHC Class I alpha peptide (aas 49-60) with a G56A substitution with the first three aas constituting the anchor modification (ADAAPWIEQEAPEYW, SEQ ID NO:357), which binds to DQ2 proteins tightly. See, e.g., Stepniak et al., J. Immnol., 180:3268-3278 (2008). In the α1 domain positions 52 and 74 are within parentheses and position 47 is bracketed for emphasis. Position 5 of the β1 domain and position 83 of the α1 domain, which may be substituted with cysteines to form body disulfide bonds, are marked with asterisks (*). All five constructs comprised R52H an S741 substitutions in their α1 domains. The control constructs lacking the body disulfide bond had C47 of the α1 domain substituted by either a Ser (C47S) in construct 3832 or a Lys in construct 3833. Each of constructs 3834, 3835, and 3836 has a disulfide bond between cysteines substituted at position 5 (E5C) of the β1 domain and position 83 (A83C) of the α1 domain, with position 47 being the wt. cysteine in construct 3834, a Ser in construct 3835, and a Lys in construct 3836 (see FIG. 18).

Constructs 3832-3836 were expressed in CHO cells and the soluble protein collected and purified on protein A columns followed by SCE. The resulting protein was subject to size separation chromatography under non-reducing conditions as shown in FIG. 19 at A. The vertical dashed line across the chromatograms shows the position where the peak of intact CIICs (as duplexes) elute. The chromatograms show that in the absence of a stabilizing disulfide bond (constructs 3832 and 3833) the protein is both degraded and partially aggregated, with the yield being 65 and 57 mg/I of culture but only about 10% in the intact duplex. The addition of a disulfide bond in the absence of a substitution to remove the cysteine at position 47 of the DQA1*05:01 α1 domain, as in construct 3834, reduces both the aggregation and degradation of the CIIC with the overall yield being 108 mg/I. Inclusion of both a disulfide bond and replacement of Cys 47 with a Ser (C47S in construct 3835) or replacement of Cys 47 with a Lys (C47K in construct 3836) results in increased yields (239 and 223 mg/I) with ˜80% of the protein under the main peak representing intact duplex CIIC. The electrophoresis gels stained with coomassie blue show the protein A and SCE purified constructs gave a single band for the constructs 3835 and 3836 separated under reducing and non-reducing conditions alongside molecular weight markers. Expected molecular weight for the reduced material 75 kDa (FIG. 19 at B).

Quantitative comparison of the expression and purification of CIICs 3835 and 3836, which differ by only the substitution at C47 of the α1 domain (3835 have a C47S and 3686 a C47K substitution indicated that the CIICs accumulated in culture media at concentrations of 222 mg/I and 209 mg/I of culture media respecitively. Purification indicated that about 80% and about 100% of the proteins were unaggregated following protein A and size exclusion chromatography (“SEC”) respectively (see Table 4).

Similar results are obtained for CIICs with linker disulfide bonds in place of body disulfide bonds.

TABLE 4
Protein Characterization of Body Disulfide Incorporation

| | 3835 (C47S | 3836 (C47K |
|---|---|---|
| Construct IDs | substitution) | substitution) |
| Serotype | DQ2.5 | DQ2.5 |
| Peptide Epitope | Class I alpha | Class I alpha |
| | (49-60; G56A) | (49-60; G56A) |
| MOD | None | None |
| Protein A yield (mg/L) | 222 | 209 |
| % Unaggregated duplex protein | 78 | 81 |
| after protein A chromatography | | |
| Final yield (mg/L) | 82 | 81 |
| % unaggregated duplex | 100 | 100 |
| protein from SEC | | |

### Example 2 Biophysical Characterization

Samples of CIICs 3835 and 3836 in duplex form from Example 1 were subjected to a series of biophysical characterizations. Freeze thaw testing (−80° C. three cycles in PBS plus saline pH 7.4), which indicated no change in aggregation state based on size separation chromatography (e.g., using integration of the peak areas with detection at 280 nm). The melting point of the MHC (HLA) domain was greater than 60° C. (65° C. in the presence of a C47S substitution and 72.6° C. in the presence of a C47K substitution), and the onstet of melting greater than 45° C., with the minimum value approaching 50° C. (at least about, or greater than 49° C.). The IgG scaffold melted above 80° C. The proteins were stable to aggregation upon heating to greater than 60° C. Accelerated stability testing of the proteins in at 42° C. indicated that proteins were substantially stable over 10 days, with construct 3835 showing greater than 80% stability and 3836 greater than 90% sstability at 10 days based on size separation chromatography (e.g., using integration of the peak areas with detection at 280 nm). Table 5 and FIG. 19 at C. The comparitive data on CIICs 3835 and 3836 emphasize, among other things the thermal stability of CIICs of this disclosure, and the contribution to that stability provided by adding a positively charged aa residue to the α1 domain at position 47.

TABLE 5
Characterization of Constructs

| | 3835 (C47S | 3836 (C47K |
|---|---|---|
| Construct IDs | substitution) | substitution) |
| Freeze-thaw (3x) | No change | No change |
| Tm (C) Class II sequence | 65 | 72.6 |
| Tm (C) IgG scaffold | 81 | 82.8 |
| Tm₁ onset | 49.9 | 71.2 |
| T_{agg} | 61.5 | 68.5 |

The melting point (Tm) and onset of melting (Tm₁) of portions of the CIICs were determined by differential scanning calorimetry using a Malvern Panalytical MicroCal DSC. Samples were diluted to 1 mg of protein/mL in PBS plus saline pH 7.4 prior to DSC analysis. DSC carried out from 20° C. to 100° C., with a scan rate of 60° C./hr, against corresponding reference buffer. Melting plots were analyzed and fitted using MicroCal PEAQ-DSC software. (Feedback mode set to “Low”). The onset temperature is the temperature at which the DSC curve initially inflects as determined by the instrument software.

The temperature of aggregation (Tagg) is determined using the change in optical transmission/absorbance caused by scattering due to aggregation of the protein sample was measured using a Nanotemper Prometheus instrument (NanoTemper Technologies GmbH, Flöβergasse 4, 81369 München, Germany). The reported Tagg is the temperature at which the second derivative of transmission vs temperature line first has a significant inflection as determined by the manufacturer's software. Unless stated otherwise, all measurements were made at 10 mg of protein/ml in PBS plus saline pH 7.4 saline from 20° C. to 95° C., with a scan rate of 60° C./hr (see Example 4 for the composition).

### Example 3 CIIC Substitutions and Expression Levels

A series of soluble duplex CIICs and duplexes of control split chain MHC constructs were prepared to examine the effect of body disulfide formation and substitutions at various locations in Class II molecules. HLA DQ 2.5 (HLADQB*2:01 and HLA DQA1*5:01) was employed as the source of the α1, α2, β1, and β2 domains because of its instability, and the peptide epitope utilized was a variant of an MHC Class I alpha peptide (aas 49-60 with a G56A) substitution (APWIEQEAPEYW, SEQ ID NO:356), which binds to DQ2 proteins tightly. See, e.g., Stepniak et al., J. Immnol., 180:3268-3278 (2008). When present, the body disulfides were formed between cysteines substituted at position 5 of the β1 domain and position 83 of the α1 domain (E5C and A83C). The effect of additional substitutions at positions C47, R52, and S74 were examined for their contribution to the expression and stability of CIICs and control split chain constructs. The constructs and their substitutions are summarized in Table 6 and the aa sequences are provided in FIG. 18. The overall structure of the CIICs is provided in FIG. 20 at A, and the control split chain constructs in FIG. 20 at B. The dashed lines between the IgG Fc scaffolds represent stabilizing disulfide bonds and the dashed lines between the α1 and β1 domain sequences represent the location of body disulfide bonds that may be present.

TABLE 6
Summary of Constructs and Substitutions

| Construct | MW | 4065 | 2804-2814 | 4057-4058 | 4055-2814 | 4056-2814 | 3439-2814 | 4059 | 4060 | 4061 | 4062 | 4063 |
|---|---|---|---|---|---|---|---|---|---|---|---|---|
| Gel Lane | 0 | 1 | 2 | 3 | 4 | — | — | 5 | 6 | 7 | 8 | — |
| Native | | | X | | | | | | | | | |
| Single Chain | | X | | | | | | X | X | X | X | X |
| CIICS | | | | | | | | | | | | |
| Split Chain | | | X | X | X | X | X | | | | | |
| Control | | | | | | | | | | | | |
| Body Disulfide | | X | | X | | | | | X | | X | X |
| C47K | | X | | | X | | | | | X | X | X |
| R52H | | X | | | | X | | | | | | X |
| S74I | | X | | | | | X | | | | | |

All CIIC and control constructs were expressed in CHO cells and purified on protein A columns followed by size separation chromatography. Samples of representative constructs (2 μg of protein) were subject to non-reducing gel electrophoresis, alongside reduced molecular weight standards (weight in kDa) and the resulting gel stained with coomassie blue is shown in FIG. 20 at C. The arrow to the right provides the location of unaggregated duplex CIIC constructs; the strong bands below it in lanes 1 and 8 are unduplexed CIICs. The results indicate that the highest levels of expression without aggregation or degradation are achieved in single chain CIIC format when they are disulfide stabilized by a body disulfide (or linker disulfide) and have a substitution at HLA DQ α1 C47 (e.g., C47K of HLA DQ constructs) or the equivalent positions in HLA DR constructs) are also present. Substitutions at positions R52 and/or S74 of DQ2.5's α1 domain (e.g., R52H and S741), and the corresponding positions in DR and DP alleles, contribute to protein stability (e.g., thermal stability) and reduce nonspecific aggregation, but contribute less to expression levels.

In order to confirm the folding of the CIICs in an active form an anti-HLA DQ Antibody SPV-L3 (Novus Biologicals Centennial, CO, USA), which recognizes the intact native DQ2.5 ap heterodimer, but not denatured or misfolded DQ2.5 protein, was used to probe the structure of CIIC 4062. As controls, loading buffer and a non-DQ2.5 constructs was employed: (i) an MHC construct comprising a Class I HLA A*02:01 heavy and an Epistine Barr Virus E7 peptide epitope (constructs 1644-1274). The 4062 CIIC and control construct had an IgG1 scaffold permitting immobilzation using anti-IgG1 antibodies.

| Construct | Description |
|---|---|
| 4062 | Class I alpha (49-60; G56A)-(G₄S)₃-hDQ2.5-beta1-2(E5C)- |
| | (G₄S)₅-alpha1-2(C47K, A83C)-GGSAAAGG-hIgG1(LALA) |
| 1644-1274 | HLA*A02(D1; A236C)-AAAGG-hIgG1(L234A, L235A), |
| | E7 11-20 -human β2microglobulin (R12C) |

SPV-L3 binding analysis was conducted using a Sartorius Octet (Bio-layer interferometry or “BLI”) system and an anti-human IgG Fc capture (“AHC”) biosensors. The AHC biosensors were loaded with the CIICs or contol constructs. For the additional loading buffer control a biosensor was also exposed to the buffer used to load the CIICs and control constructs. After removing unbound CIICs and control constructs, SPV-L3 antibody was added to loaded AHC sensors at 125, 250, or 500 nM and the response of the BLI insrument monitored. The 4062 CIIC produced an immediate time dependent response that asymptomatically approached its maximum by about 300 sec (5 min) at 500 nM of antibody. SPV-L3 antibody did not produce a BLI response indicative of SPV-L3 antibody binding to either the control constructs or the control buffer indicating the presence of DQ 2.5 containing constructs folded in active form. Data not shown.

### Example 4 Activity and Specificity of CIICs

Three soluble MOD-less CIIC constructs 4062, 4149, and 4214 were used to assess CIIC folding into an active form and each CIICs ability to present an epitope to a TCR specific for the peptide epitope. All three CIICs had the structure (peptide epitope)-(G₄S)₃-hDQ2.5-beta1-2(E5C)-(G₄S)₅-alpha1-2(C47K, A83C)-GGSAAAGG-hIgG1(LALA), with each of the three constructs having a β1 (E5C) to α1 (A83C) body disulfide, but bearing a different epitope as indicated in Table 7 and FIG. 18. The peptide epitope, purification data, thermal characterization data in phosphate buffered saline (PBS plus saline) are provided in Table 7. SDS PAGE gels of the protein A and SEC purified protein gave a single band under both reducing and non-reducing conditions (not shown). Unless stated otherwise the composition of PBS plus saline employed herein is 10.1 mM dibasic sodium phosphate (Na₂HPO₄), 1.76 mM monobasic potassium phosphate (KH₂PO₄), 2.7 mM KCl, and 500 mM NaCl with a final pH of 7.4.

TABLE 7
MOD-less CIIC Construct Epitopes

| Construct IDs | 4062 | 4149 | 4214 |
|---|---|---|---|
| Epitope Name | MHC Class I alpha | Alpha 1a Gliadin | Alpha 2 Gliadin |
| | (49-60; G56A) | | |
| Epitope Sequence | APWIEQEAPEYW | QLQPFPQPELPY | QPFPQPELPYPQPE |
| | (SEQ ID NO: 356) | (SEQ ID NO: 175) | (SEQ ID NO: 176) |
| Yield after Protein A | 69 mg/l | 11.6 mg/l | 26.1 mg/l |
| and SEC | | | |
| % unaggregated duplex | 100 | 97.8 | 98.9 |
| protein from SEC | | | |
| Freeze-thaw (3x) | Stable-No change | Stable-No change | Stable-No change |
| Tm (C) Class II sequence | 72.1 | 67.8 | 65 |
| Tm (C) IgG scaffold | 81.8 | 81.4 | 81.4 |
| Intact Mass (by LC MS) | Confirmed | Confirmed | Confirmed |

The data indicated all three proteinsDwere stable to three rounds offreeze thaw testing (−80° C. three cycles in PBS plus saline pH 7.4 saline) and showed no substantive change in aggregation state based on size chromatography. The melting point ofthe MHC (HLA) domain was greater than 60° C. (greater than 65° C.). The IgG scaffold melted above 80° C. Liquid chromatography mass spectroscopy (LC MS) confirmed that the proteins had the correct molecular weight. Tm values were determined as in Example 2.

### Example 5 Native Folding of CIICs

In order to confirm the folding of the CIICs in an active form an anti-HLA DQ Antibody SPV-L3 (Novus Biologicals Centennial, CO, USA), which recognizes the intact native DQ2.5 α13 heterodimer, but not denatured or misfolded DQ2.5 protein, was used to probe the structure of CIICs 4062, 4149 and 4214 (see Example 4 and Table 8). As controls, loading buffer and two non-DQ2.5 constructswere employed: (i) an MHC constructcomprising a Class I HLA A*02:01 heavy and Epstine Barr Virus E7 peptide epitope (construct 1644-1274); and (ii) a Class II protein construct comprising a human DR4 (DRA*01:01 a domains and DRB*04:01 β domains) and a proinsulin peptide epitope (construct 2932-2639). All ofthe CIICs and control constructs had an IgG scaffold permitting immobilzation using anti-IgG1 antibodies.

TABLE 8
Description of Constructs

| Construct | Description |
|---|---|
| 4062 | Class I alpha (49-60; G56A)-(G₄S)₃-hDQ2.5-beta1-2(E5C)-(G₄S)₅-alpha1- |
| | 2(C47K, A83C)-GGSAAAGG-hIgG1(LALA) |
| 4149 | Alpha1a GLIADIN-(G₄S)₃-hDQ2.5-beta1-2(E5C)-(G₄S)₅-alpha1-2(C47K, A83C)- |
| | GGSAAAGG-hIgG1(LALA) |
| 4214 | Alpha2 GLIADIN-h-(G₄S)₃-hDQ2.5-beta1-2-(G₄S)₅-(A83C:E5C)alpha1-2(C47K)- |
| | GGSAAAGG-hIgG1(LALA) |
| 2932- | Native leader-hDRA1*0101-alpha1-2-GGSAAAGG-hIgG1(LALA), Proins |
| 2639 | (76-90; K88S)-(G₄S)₃-hDRB1*0401-beta1-2 |
| 1644- | HLA*A02(D1; A236C)-AAAGG-hIgG1(L234A, L235A), E7 11-20-human |
| 1274 | β2microglobulin (R12C) |

(G₄S = SEQ ID NO: 237; GGSAAAGG = SEQ ID NO: 249)

SPV-L3 binding analysis was conducted using a Sartorius Octet (Bio-layer interferometry or “BLI”) system and an anti-human IgG Fc capture (“AHC”) biosensors. The AHC biosensors were loaded with the CIICs or contol constructs. For the additional loading buffer control a biosensor was also exposed to the buffer used to load the CIICs and control constructs. After removing unbound CIICs and control constructs, SPV-L3 antibody was added to loaded AHC sensors at 125, 250, or 500 nM and the response of the BLI insrument monitored. All three CIICs produced an immediate time dependent response that asymptomatically approached its maximum by about 300 sec (5 min) at 500 nM of antibody. SPV-L3 antibody did not produce a BLI response indicative of SPV-L3 antibody binding to either the control constructs or the control buffer indicating the presence of DQ 2.5 containing constructs folded in active form. Data not shown.

### Example 6 Expression of CIIC with HLA DP Subunits

Two soluble MOD-less CIIC constructs 4104 and 4105, and two MOD-containing soluble CIICs 4351 and 4354 were used to assess the production of CIICs comprising HLA DP subunits (sequences are provided in FIG. 18. All four constructs had an N-terminal epitope and a common the core structure: (epitope)-(G₄S)₃-hDPB1*04:01-beta 1(E5C)— beta 2-(G₄S)₅-hDPA1*01:03-alpha 1(Q81C)-alpha 2-GGSAAAGG-hlgG1(LALA); with a β1 (E5C) to α1 (Q81C) body disulfide. The MOD-containing constructs included on their C terminus the two copies of a variant human IL-2 sequence in tandem “-(G₄S)₃-2xhlL2(H16A, F42A).” The epitope peptides were from human telomerase reverse transcriptase (hTERT) or melanoma antigen (MAGE). An “h” preceding various elements such as the IgG and MHC sequences indicates the sequences are of human origin.

The CIIC constructs expressed in COS cells and purified using protein A and SEC. Size The purification summary and biophysical analysis are set forth in Table 8a. Unless stated otherwise the analyses were conducted in phosphate buffered saline (PBS plus saline) with 500 mM NaCl having the composition of: 10.1 mM dibasic sodium phosphate (Na₂HPO₄), 1.76 mM monobasic potassium phosphate (KH₂PO₄), 2.7 mM KCl, and 500 mM NaCl with a final pH of 7.4.

TABLE 8a

| | | | 4351 | 4354 (h IL-2 |
|---|---|---|---|---|
| | 4104 | 4105 | (MOD- | H16A, F42A |
| Construct IDs | (MOD-less) | (MOD-less) | containing) | MOD-containing) |
| Peptide Epitope Name | hTERT(616-626) | MAGE-A3 | MAGE-A3 | hTERT(616-626) |
| | | (247-258) | (245-258) | |
| Epitope Sequence | | | | |
| Expression conc. | 264.8 mg/l | 236.2 mg/l | 284.4 mg/l | 330.4 mg/l |
| Yield after Protein A | 103 mg/l | 70 mg/l | 119.5 mg/l | 118.5 mg/l |
| and SEC | | | | |
| Endotoxin (endotoxin | <0.1 | <0.1 | <0.1 | <0.1 |
| units/mg) | | | | |
| % unaggregated duplex | 99 | 99 | 98.4 | 98.4 |
| protein from SEC | | | | |
| Freeze-thaw (3x) | Stable - | Stable - | Stable - | Stable - |
| | No change | No change | No change | No change |
| Tm1 (C) Class | 45.4 | 48.6 | n/a | 45.8 |
| II sequence | | | | |
| Tm2 (C) Class | 51.6 (valley) | n/a | 54.3 | 52.8 |
| II sequence | | (not applicable) | | |
| Tm3 (C) Class | 67 | 66.5 | 66.6 | 67.2 |
| II sequence | | | | |
| Tm4 (C) IgG | 80.6 | 80.3 | 82.6 | 82.8 |
| scaffold | | | | |
| Tagg (° C.) | 45.1 | 48 | 49.6 | 47.6 |
| Intact Mass | Confirmed | Confirmed | Confirmed | Confirmed |
| (by LC MS) | | | | |

The data indicated all three proteins were stable to three rounds of freeze thaw testing (−80° C. three cycles in PBS pH 7.4 saline) and showed no substantive change in aggregation state based on size chromatography. The melting point of the MHC (HLA) domain showed several transitions that weree greater than 45° C. The IgG scaffold melted above 80° C. The initial temperaterature at which aggregation was observed (Tagg) is greater than 45° C. Liquid chromatography mass spectroscopy (LC MS) confirmed that the proteins had the correct molecular weight. Where n/a appears in Table 8a the data are not applicable. Tm and Tagg values were determined as in Example 2.

### Example 7 Functional Activity of CIICs (Stimulation of CD69 Expression)

In order assess the ability of CIICs to present peptide epitopes to a TCRs in a peptide epitope specific fashion, SKW-3 T cells into which the specific TCRs have been introduced were employed. CD69 expression was used as marker for peptide epitope specific TCR binding, engagement, and T cell activation. For the assays CIIC constructs comprising IgG Fc scaffolds were presented to the SKW-3 cells by Raji cells that bind the IgG Fc scaffolds. CD69 expression by the SKW-3 cells was assessed by Fluorescence Activated Cell Sorting (FACS).

A. Binding Activation of a T cell by a CIIC through an Epitope Specific TCR

SKW-3 cells presenting TCR #S16 specific for the gliadin α2 peptide epitope (QPFPQPELPYPQPE, SEQ ID NO:176) were contacted with Raji cells previously exposed to and presenting various amounts of either CIIC construct 4214 bearing the α2 peptide epitope or CIIC construct 4149 bearing the α1a epitope (QLQPFPQPELPY, SEQ ID NO:175) see Example 4. The results presented in FIG. 24 at A demonstrate that SKW-3 cells bearing TCR #S16 bind and are activated by CIIC 4214, which presents the gliadin α2 peptide epitope in a dose dependent manner over the range of 0-5,000 nM of that CIIC, but not by CIIC 4149 that presents the gliadin α1a peptide epitope. The lack of CD69 expression using free peptide epitope (Irr. peptide) and the maximal response achieved with phorbol myristic acid (PMA) controls are indicated for reference.

B. CIICs Produce Binding and Activation of T cells in an Epitope Specific Manner

Tissue culture plates were prepared containing Raji cells incubated for 24 hrs. with various concentrations of MOD-less CIIC construct 4149 (presenting the α1a peptide), MOD-less CIIC construct 4214 (presenting the α2 peptide), or MOD-less CIIC construct 4062 (presenting a control peptide), or various concentrations of the α1a or α2 gliadin peptides from 0-1,000 nM. To the Raji cells were added SKW-3 cells expressing TCR #380 specific for the gliadin α1a epitope or TCR #S26 specific for the gliadin α2 epitope. After 24 hrs. the expression of CD69 by the SKW-3 cells was determined by FACS. The results shown in FIG. 24 at B demonstrate the CIICs provide a concentration dependent epitope specific TCR/T cell activation as SKW-3 cells expressing TCR #S16 were activated only by CIIC 4214, and not by CIIC 4149 or any of the controls. In contrast, SKW-3 cells expressing TCR #380 were activated (expressed CD69) in response to the α1a peptide and not by CIIC 4149 comprising that peptide or the 4214 any of the controls. An expanded dose response curve for action of construct 4149 on SKW-3 cells expressing TCR #380 is provided in FIG. 24 at structure C confirming that construct does not activate the SKW-3 cells.

### Example 8 Anchor Modifications and N- or C-Terminal Extensions

In order to determine the effect that various anchor position modifications and N-terminal and/or C-terminal extensions have on the expression of CIIC a single HLA DQ2.5 CIIC twenty different constructs bearing control epitopes or celiac peptide epitopes were prepared. The core construct had the overall structure: Epitope-(G₄S)₃— β1 domain (E5C)—β2 domain—(G₄S)₅— α1 domain (C47K), A83C)—α2 domain—GGSAAAGG linker—human IgG1 with LALA substitutions with a body disulfide bond between the E5C and A83C substitutions, and the sequence: (epitope)-GGGGSGGGGSGGGGSRDSPC*DFVYQFKGMCYFTNGTERVRLVSRSYNREEIVRFDSDVGEFRAVTLL GLPAAEYWNSQKDILERKRAAVDRVCRHNYQLELRTTLQRRVEPTVTISPSRTEALNHHNLLVCSVTDFYPAQIKVR WFRNDQEETAGVVSTPLIRNGDWTFQILVMLEMTPQRGDVYTCHVEHPSLQSPITVEWRAQSESAQSKGGGGSGG GGSGGGGSGGGGSGGGGSEDIVADHVASYGVNLYQSYGPSGQYTHEFDGDEQFYVDLGRKETVWKLPVLHQFRF DPQFALTNIAVLKHNLNILIKRSNSTC*ATNEVPEVTVFSKSPVTLGQPNILICLVDNIFPPVVNITWLSNGHSVTEGVSE TSFLSKSDHSFFKISYLTLLPSAEESYDCKVEHWGLDKPLLKHWEPEIPAPMSELTEGGSAAAGGDKTHTCPPCPAP EAAGGPSVFLFPPKPKDTLMISRTPEVTCVVVDVSHEDPEVKFNWYVDGVEVHNAKTKPREEQYNSTYRVVSVLTVL HQDWLNGKEYKCKVSNKALPAPIEKTISKAKGQPREPQVYTLPPSREEMTKNQVSLTCLVKGFYPSDIAVEWESNGQ PENNYKTTPPVLDSDGSFFLYSKLTVDKSRWQQGNVFSCSVMHEALHNHYTQKSLSLSPG (SEQ ID NO:154). The substituted cysteine residues marked by an asterisk (*) form a body disulfide bond. The sample number, construct number and epitope are provided in Table 9. Samples 1-10 are based on α-1 gliadin, samples 11-17 are based on a-2 gliadin, samples 18-20 are control constructs with a Class I alpha surrogate peptide. All of the constructs have a C47K substitution except 3834, which retains the wild-type cysteine in the α1 domain and 3947 which has a C47R substitution.

TABLE 9
Sample number, Construct number and Epitope

| Sample | Construct | Epitope | Sample | Construct | Epitope |
|---|---|---|---|---|---|
| 1 | 3940 | QLQPFPQPELPY {175} | 11 | 3950 | PQPELPYPQPE {177} |
| 2 | 3941 | LQPFPQPELPY {292} | 12 | 3951 | QPQPELPYPQPE {187} |
| 3 | 3942 | ADAQLQPFPQPELPY | 13 | 3952 | ADAQPQPELPYPQPE |
| | | {293} | | | {300} |
| 4 | 3943 | ADALQPFPQPELPY | 14 | 3953 | ADAPQPELPYPQPE {301} |
| | | {294} | | | |
| 5 | 3944 | ADAQPFPQPELPY {295} | 15 | 3954 | IQPELPYPQPE {302} |
| 6 | 3945 | ADAPFPQPELPY {296} | 16 | 3955 | PQPELPEPQPE {303} |
| 7 | 3946 | QLQIFPQPELPY {297} | 17 | 3956 | IQPELPEPQPE {188} |
| 8 | 3947 | QLQPFPEPELPY {298} | 18 | 3957 (C47R) | ADAAPWIEQEAPEYW |
| | | | | | {357} |
| 9 | 3948 | QLQPFPQPEEPY {299} | 19 | 3834 (C47 is | ADAAPWIEQEAPEYW |
| | | | | unsubstituted) | {357} |
| | | | | {124} | |
| 10 | 3949 | QLQIFPEPEEPY {186} | 20 | 3836 (C47R) | ADAAPWIEQEAPEYW |
| | | | | {126} | {357} |

The underlined portions of the epitopes represent the core epitope. Anchor modifications are bolded and italicized. SEQ ID NOs are provided in brackets “{ }” following the construct number or peptide epitope sequence.

The constructswere expressed in CHO cells in 96 well plates and the concentrationd ofthe expressed proteins were determined (see the histogram in FIG. 27 at A). Samples of six proteins from constructs 3940, 3949, 3951, 3956, 3957, and 3836 were expressed and purified on protein A. Chromatograms of samples from protein A are provided in FIG. 27 panel B indicate a high level of protein expression (in duplex form) that is otherwise substantially unaggregated or degraded. The proteins appear substantially as single band on reducing and non-reducing SIDS page gels (data not shown). Purification and stability data are provided in Table 10.

TABLE 10
Purification and stability

| | | | ProtA | | | |
|---|---|---|---|---|---|---|
| | | | yield | | Final | |
| | | | AKTA | SEC | % | |
| Con- | | MW | OD280 | yield | mono- | Freeze |
| struct | Peptide | (kDa) | (mg/L) | (mg/L) | mer | thaw |
| 3940 | Alpha1a GLIADIN | 75 | 47.3 | 7.5 | 91.4 | Pass |
| | (QLQPFPQPELPY, | | | | | |
| | SEQ ID | | | | | |
| | NO: 175) | | | | | |
| 3949 | Alpha1a | 75 | 147 | 59 | 98.3 | Pass |
| | GLIADIN-j | | | | | |
| | (anchor mod | | | | | |
| | QLQIFPEPEEPY, | | | | | |
| | SEQ ID | | | | | |
| | NO: 186) | | | | | |
| 3951 | Alpha2 | 75 | 58.5 | 9.5 | 97.1 | Pass |
| | GLIADIN-b | | | | | |
| | (QPQPELPYPQPE, | | | | | |
| | SEQ ID | | | | | |
| | NO: 187) | | | | | |
| 3956 | Alpha2 | 74 | 106 | 26.5 | 97.6 | Pass |
| | GLIADIN-g | | | | | |
| | (anchor mod | | | | | |
| | IQPELPEPQPE, | | | | | |
| | SEQ ID NO: 188) | | | | | |
| 3957 | ADA-Classlalpha | 75 | 238 | 89 | 100 | Pass |
| | (49-60; G56A) | | | | | |
| | (with C47R, | | | | | |
| | R52H, S74I) | | | | | |
| 3836 | ADA-Classlalpha | 75 | 250.5 | 108 | 100 | Pass |
| | (49-60; G56A) | | | | | |
| | (with C47K, | | | | | |
| | R52H, S74I) | | | | | |

### Example 9 CIIC with ω Gliadin Epitopes

In order to determine if the C110 constructs found to be effective for the production and presentation of alpha 1a gliadin epitopes and alpha 2 gliadin epitopes could be extended to w gliadin epitopes four constructs (4156-4159) comprising differing native w gliadin epitopes were prepared. The constructs have the same overall structure as those in Example 7: Epitope-(G₄S)₃— 131 domain (E5C)—β2 domain—(G₄S)₅— α1 domain (A83C)—α2 domain —GGSAAAGG —human IgG1 with LALA substitutions with a body disulfide bond between the E5C and A83C substitutions. The w gliadin constructs were compared to some new and previously tested alpha 1 a gliadin epitopes and alpha 2 gliadin epitopes (see Example 7). A control “C” comprising the surrogate epitope, MHC Class I alpha_{49-60,G56A} was also included in the comparison. The samples and epitopes are listed Table 11:

TABLE 11
CIIC Samples with ω Gliadin Epitopes

| Sample | Construct | Epitope | Sample | Construct | Epitope |
|---|---|---|---|---|---|
| 1 | 3940 | QLQPFPQPELPY {175} | 10 | 3950 | PQPELPYPQPE {177} |
| 2 | 3941 | LQPFPQPELPY {292} | 11 | reference | APQPELPYPQPE {307} |
| | | | | not | |
| | | | | provided | |
| 3 | 3946 | QLQ/FPQPELPY {297} | 12 | 3954 | IQPELPYPQPE {302} |
| 4 | 3947 | QLQPFPEPELPY {298} | 13 | 3955 | PQPELPEPQPE {303} |
| 5 | 3948 | QLQPFPQPEEPY {299} | 14 | 3956 | IQPELPEPQPE {188} |
| 6 | 3949 | QLQ/FPEPEEPY {186} | 15 | 4157 | QPEPFPQPEQPFPW {309} |
| 7 | reference | QPFPQPELPYPQPE {176} | 16 | 4158 | PEPFPQPEQPFPW {310} |
| | not | | | | |
| | provided | | | | |
| 8 | reference | PFPQPELPYPQPE {304} | 17 | 4159 | EPFPQPEQPFPW {311} |
| | not | | | | |
| | provided | | | | |
| 9 | reference | FPQPELPYPQPE (306} | 18 | 4156 | PFPQPEQPFPW {312} |
| | not | | | | |
| | provided | | | | |

The underlined portions of the epitopes represent the core epitope. Anchor modifications are bolded and italicized. SEQ ID NOs are provided in braces “{ }” following the construct numbers and epitopes.

The constructs were expressed in CHO cells in 96 well plates and the concentration of the expressed proteins were determined (see FIG. 28). The results indicate that some w gliadins can be expressed at levels comparable to those obtainable with alpha 1a gliadin epitopes and alpha 2 gliadin epitopes.

### Example 10 Additional Constructs

Two additional MOD-less constructs employing HLA DRA and DDRB alleles were prepared. The constructs both have the overall structure: epitope-linker-DRB*0401 131(P5C )β2—linker—DRA1 α1 (P81C) α2 (α2 membrane proximal region)—GGSAAAGG linker—human (h)IgG1 (LALA), and the sequence:

(SEQ ID NO: 155)
(epitope)-GGGGGGGGSGGGGSGDTRC*RFLEQVKHECHFFNGTERV
RFLDRYFYHQEEYVRFDSDVGEYRAVTELGRPDAEYWNSQKDLLEQKRA
AVDTYCRHNYGVGESFTVQRRVYPEVTVYPAKTQPLQHHNLLVCSVNGF
YPASIEVRWFRNGQEEKTGVVSTGLIQNGDWTFQTLVMLETVPRSGEVY
TCQVEHPSLTSPLTVEWRARSESAQSKMGGGGSGGGGSGGGGSGGGGSG
GGGSIKEEHVIIQAEFYLNPDQSGEFMFDFDGDEIFHVDMAKKETVWRL
EEFGRFASFEAQGALANIAVDKANLEIMTKRSNYTC*ITNVPPEVTVLT
NSPVELREPNVLICFIDKFTPPVVNVTWLRNGKPVTTGVSETVFLPRED
HLFRKFHYLPFLPSTEDVYDCRVEHWGLDEPLLKHWEFDAPSPLPETGG
SAAAGGDKTHTCPPCPAPEAAGGPSVFLFPPKPKDTLMISRTPEVTCVV
VDVSHEDPEVKFNWYVDGVEVHNAKTKPREEQYNSTYRWSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPG.

The melting point of the MHC (HLA) domain showed several transitions that were greater than 45° C. The initial temperaterature at which aggregation was observed (Tagg) is greater than 45° C. Liquid chromatography mass spectroscopy (LC MS) confirmed that the proteins had the correct molecular weight. Where n/a appears in Table 8a the data are not applicable. Tm and Tagg values were determined as in Example 2.

The first of the two constructs, 3922 comprised a human proinsulin (Proins) sequence (aas 76-90) with a K88S substitution (SLQPLALEGSLQSRG) (SEQ ID NO:281). The second of the two constructs, 3926 comprised a glutamic acid decarboxylase (GAD65) sequence from (aas 555-567) with a F5571 substitution (NFIRMVISNPAAT) (SEQ ID NO:280). Both constructs gave a single band on SDS page gels after purification on protein A followed by size separation chromatography.

### Example 11 Allelic Diversity, MOD Diversity and Antigen Diversity

Three groups of constructs were prepared to examine the effects of A. Allelic diversity, B. MOD diversity, and C. Antigen diversity on expression levels of CIIC constructs.

In Group A four MOD-less duplex-forming CIIC constructs from DQ, DP, DR4, DR15 and DP alleles were prepared to demonstrate the ability to express CIIC constructs having HLA DP, DQ, or DR sequences. Construct 4102 comprises an MHC class 1 alpha epitope presented by HLA DQ2.5 sequences. Construct 4105 comprises a melanoma antigen (MAGE) epitope presented by DP4 sequences. Construct 4433 comprises a Timothy Grass (Phleum pratense) or “Tim Grass” pollen peptide epitope presented by HLA DR4 sequences. Construct 4434 comprises myelin basic protein (“MBP”) epitope presented by HLA DR15 sequences. The constructs have the overall structure epitope—linker—HLA β1—HLA β2—HLA α1—HLA α2—linker-Human IgG1 Fc (typically with LALA substitutions). The sequence of the constructs are provided in FIG. 18. Each of the constructs were expressed and purified on Protein A followed by size separation chromatography. Each of the constructs resulted in a single band on SDS PAGE gels.

In Group B five duplex CIIC constructs having different MODs or combinations of MODs were prepared. Each of the constructs comprised DR4 sequences presenting a TimGrass (233-245) epitope (ESYKFIPALEAAV) (SEQ ID NO:358), and have the overall structure: epitope-DR4 (p1(P5C)-β2-α1-α2-GGSAAAGG linker-Human IgG1 Fc (LALA)-MOD. Construct 4436 comprises an IL-2 MOD bearing H16A and F2A substitutions. Three of the constructs (4107, 4436, and 4441) employed non-interspecific scaffold sequences. Construct 4107 comprised a human (h)PDL-1 MOD, 4436 comprised an IL-2 MOD, and 4441 comprised a human IL-10 MOD. Two duplex CIIC constructs were formed from pairs of interspecific constructs (pair 4438/4439 and pair 4438/4440) that share a common 4438 CIIC. The interspecific constructs each comprised a TGF p3 MOD on the common 4438 CIIC masked by at TBRII sequence in trans, with the 4438/4440 pair further comprising an H16A F42A substituted IL-2 sequence as part of CIIC 4440. Each of the constructs were expressed and purified on Protein A followed by size separation chromatography. Each of the constructs resulted in a single band on SDS PAGE gels. The 4438 and 4439 construct pairwere too similar in molecularweight to resolve at the level ofgel loading. The 4438/4440 pairwere visably resolved due to the difference in molecular weight resulting from the IL-2 sequence.

In Group C eleven (11) MOD-less CIICs having a common structure but presenting different peptide epitopeswere prepared to test the antigen diversity that can be incorporated into a CIIC while retaining expression. The MOD-less CriCs were constructed having the overall structure: epitope-(G₄S)₃—HLA DR1 *0401(β1(E5C)-β2-α1 (P81C)-α2 (α2 membrane proximal region)-GGSAAAGG linker-Human IgG Fc (LALA), and the sequence:

(SEQ ID NO: 156)
(epitope)-GGGGSGGGGSGGGGSGDTR(C*)RFLEQVKHECHFFNGT
ERVRFLDRYFYHQEEYVRFDSDVGEYRAVTELGRPDAEYWNSQKDLLEQ
KRAAVDTYCRHNYGVGESFTVQRRVYPEVTVYPAKTQPLQHHNLLVCSV
NGFYPASIEVRWFRNGQEEKTGVVSTGLIQNGDWTFQTLVMLETVPRSG
EVYTCQVEHPSLTSPLTVEWRARSESAQSKMGGGGSGGGGSGGGGSGGG
GSGGGGSIKEEHVIIQAEFYLNPDQSGEFMFDFDGDEIFHVDMAKKETV
WRLEEFGRFASFEAQGALANIAVDKANLEIMTKRSNYT(C*)ITNVPPE
VTVLTNSPVELREPNVLICFIDKFTPPVVNVTWLRNGKPVTTGVSETVF
LPREDHLFRKFHYLPFLPSTEDVYDCRVEHWGLDEPLLKHWEFDAPSPL
PETGGSAAAGGDKTHTCPPCPAPEAAGGPSVFLFPPKPKDTLMISRTPE
VTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFF
LYSKLTVDKSRWQQGNVFSCSVMHEALHNHYTQKSLSLSPG.

The linker sequences are set forth in bold and italics, the Ig Fc is underlined, and a body disulfide bond is formed between the C residues marked by an asterik “(C*)”. The epitopes are as setforth in Table 12 which follows. The SEQ ID NOs of the peptide epitopes are provided in brackets “{ }”0.284). The substituted cysteine residues marked by an asterisk (*) form a body disulfide bond.

TABLE 12
Example 11 Epitopes

| Construct | Epitope | Construct | Epitope |
|---|---|---|---|
| 4451 | melanoma antigen (MAGE) A5 | 4460 | Human Immunodeficiency Virus (HIV) gag |
| | (102-116) ESEFQAALSRK {359} | | (163-182) |
| | | | AFSPEVIPMFSALSEGATPQ {365} |
| 4453 | New York esophageal squamous | 4462 | Epstein-Barr Virus Nuclear antigen 2 |
| | cell carcinoma 1 (NY-ESO1) | | (EBV-ENA2) (11-30) |
| | (125-137) LKEFTVSGNILTIRL {360} | | GQTYHLIVDTDSLGNPSLSV {366} |
| 4454 | Melanocyte protein PMEL or | 4463 | Myelin/Oligodendrocyte Glycoprotein |
| | Glycoprotein 100 (gp100) | | (MOG) (97-109) |
| | (44-59) WNRQLYPEWTEAQRLD {361} | | RTELLKDAIGEGK {367} |
| 4455 | Human Epidermal Growth Factor | 4464 | Type II Collagen alpha 1 chain (CII) |
| | Receptor 2 (HER2) (883-899) | | (259-273) |
| | KVPIKWMALESILRRRF {362} | | GIAGFKGEQGPKGEP {368} |
| 4456 | Tyrosinase (365-381) | 4465 | Type II Collagen (CII) (259-273 G262A) |
| | ALHIYMDGTMSQVQGSA {363} | | GIAAFKGEQGPKGEP {369} |
| 4459 | Hepatitis C Virus (HCV) (1248- | | |
| | 1262) GYKVLVLNPSVAATL {364} | | |

Each of the constructs were expressed and purified on protein A followed by size separation chromatography. Each of the constructs resulted in a single band on SIDS PAGE gels.

1. An MHC Class II protein construct (“CIIC”) comprising in a single aa sequence in the N-terminal to C-terminal direction:
(i) a peptide epitope aa sequence;
(ii) an Li aa linker sequence;
(iii) an MHC Class II β chain polypeptide sequence comprising a β1 and β2 domain sequence;
(iv) optionally an L2 aa linker sequence;
(v) an MHC Class II α chain polypeptide sequence comprising an α1 and α2 domain sequence;
(vi) optionally an L3 aa linker sequence;
(vii) optionally an scaffold sequence and/or MAS;
(viii) optionally an L4 linker; and
(ix) optionally one or more additional polypeptide sequences;
wherein
(i) the CIIC comprises either a body disulfide bond between the β1 domain and the α1 domain, or a linker disulfide bond between a cysteine in the L1 linker and a cysteine in the α1 domain; and
(ii) optionally, when the Class II polypeptide comprises a cysteine at aa 43 through aa 48 of the α chain polypeptide sequence (α1 and α2 domain sequence), it is substituted by an aa other than cysteine.

2. The CIIC of claim 1 wherein:
the MHC Class II β chain polypeptide sequence has at least 90% or 100% aa sequence identity to all or at least 170 contiguous aas of a DQB 31 and 32 domain sequence of DQB1*02:01, DQB1*02:02, DQB1*03:01, DQB1*03:02, DQB1*03:03, DQB1*03:04, DQB1*04:01, DQB1*04:02, DQB1*05:01, DQB1*06:01, DQB1*06:02, DQB2 isoform 1 or DQB2 isoform 2; and/or
the NMC Class II α chain polypeptide sequence has at least 90% or 100% aa sequence identity to at least 165 contiguous aas of a DQA α1 and α2 domain sequence of DQA1*05:01, DQA1*01:01, DQA1*01:02, DQA1*01:03, DQA1*01:04, DQA1*02:01, DQA1*03:01, DQA1*03:02, DQA1*04:01, DQA1*05:05, DQA1*06:01, or DQA2*01:01.

3. The CIIC of claim 1, wherein the sequences to which the DQB 31 and J2 domain sequences and the DQA α1 and α2 domain sequences have at least 90% or 100% aa sequence identity are, respectively, a DQB and DQA allele pair selected from:
(i) DQB1*02:01 and DQA1*05:01 (DQ2.5);
(ii) DQB1*02:02 and DQA1*02:01 (DQ2.2);
(iii) DQB1*03:02 and DQA1*03:01 (DQ8.1);
(iv) DQB1*04:02 and DQA1*04:01 (DQ4.2);
(v) DQB1*04:01 or DQB1*04:02 and DQA1*03:01 (DQ4.3a and 4.3b);
(vi) DQB1*05:01 and DQA1*01:01; or
(vii) DQB1*06:02 and DQA1*01:02 (DQ6.2).

4. The CIIC of claim 3, comprising:
(i) a body disulfide bond formed between the N-terminal 8 amino acids of the DQB1 or DQB2 β1 domain sequence and the C-terminal 6 amino acids of the DQA α1 domain sequence; or
(ii) a linker disulfide bond formed between a cysteine in the L1 linker sequence and a cysteine at position 76, 77, 78, or 79 of the DQAlor DQA2 α1 domain sequence.

5. The CIIC of claim 4, comprising a body disulfide bond formed between a cysteine substituted at position 5 of the DQB1 (an E5C substitution) or DQB2 (a K5C substitution) β1 domain and a cysteine substituted at position 82, 83, 84 or 85 of the DQA1 or DQA2 α1 domain sequence.

6. The CIIC of claim 5
(i) comprising a substitution at any one or more of positions 40, 52, 74 or 75 of the DQA1 or DQA2 α1 domain; and/or
(ii) wherein aa position 47 of the DQA1 or DQA2 α1 domain sequence is an aa other than cysteine.

7. The CIIC of claim 1, wherein:
(i) the NMC Class II β chain polypeptide sequence has at least 90% or 100% aa sequence identity to all or at least 170 contiguous aas of a DRB R 1 and 32 domain sequence of DRB1*01:01, DRB1*01:02, DRB1*01:03, DRB1*03:01, DRB1*03:02, DRB1*03:04, DRB1*04:01, DRB1*04:02, DRB1*04:03, DRB1*04:04, DRB1*04:05, DRB1*04:06, DRB1*04:08, DRB1*07:01, DRB1*08:01, DRB1*08:02, DRB1*08:03, DRB1*09:01, DRB1*10:01, DRB1*11:01, DRB1*11:03, DRB1*11:04, DRB1*12:01, DRB1*13:01, DRB1*13:03, DRB1*14:01, DRB1*14:02, DRB1*14:05, DRB1*14:06, DRB1*15:01, DRB1*15:02, DRB1*15:03, DRB1*15:04, DRB1*15:05, DRB1*15:06, DRB1*15:07, DRB1*16:01, DRB3*01:01, DRB3*02:01, DRB3*03:01, DRB4*01:01, DRB4*01:03, or DRB5*01:01; and
the NMC Class II α chain polypeptide sequence has at least 90% or 100% aa sequence identity to at least 165 contiguous aas of the DRA α1 and α2 domain sequence of DRA1*01:01 or DRA*01:02; and/or
(ii) the NMC Class II β chain polypeptide sequence has at least 90% or at least 95% aa sequence identity to at least 80 or at least 90 contiguous aas of a DRB 31 or 32 domain sequence of DRB1*01:01, DRB1*01:02, DRB1*01:03, DRB1*03:01, DRB1*03:02, DRB1*03:04, DRB1*04:01, DRB1*04:02, DRB1*04:03, DRB1*04:04, DRB1*04:05, DRB1*04:06, DRB1*04:08, DRB1*07:01, DRB1*08:01, DRB1*08:02, DRB1*08:03, DRB1*09:01, DRB1*10:01, DRB1*11:01, DRB1*11:03, DRB1*11:04, DRB1*12:01, DRB1*13:01, DRB1*13:03, DRB1*14:01, DRB1*14:02, DRB1*14:05, DRB1*14:06, DRB1*15:01, DRB1*15:02, DRB1*15:03, DRB1*15:04, DRB1*15:05, DRB1*15:06, DRB1*15:07, DRB1*16:01, DRB3*01:01, DRB3*02:01, DRB3*03:01, DRB4*01:01, DRB4*01:03, or DRB5*01:01, and/or
the NMC Class II α chain polypeptide sequence has at least 90% or at least 95% aa sequence identity to at least 70 or at least 80 contiguous aas of the DRA α1 or α2 domain sequence of DRA1*01:01 or DRA*01:02.

8-10. (canceled)

11. The CIIC of claim 1, wherein:
(i) the MHC Class II β chain polypeptide sequence has at least 95% or at least 98% aa sequence identity to all or at least 165 contiguous aas of a DPB R 1 and J2 domain sequence of DPB1*01:01, DPB1*02:01, DPB1*03:01, DPB1*04:01, DPB1*06:01, DPB1*09:01, DPB1*11:01, DPB1*13:01, DPB1*35:01, DPB1*71:01, DPB1*104:01, or DPB1*141:01; and/or
the MHC Class II α chain polypeptide sequence has at least 90% or 100% aa sequence identity to at least 165 contiguous aas of the DPA α1 and α2 domain sequences of DPA1*01:03 or DPA1*02:01; and/or
(ii) the MHC Class II β chain polypeptide sequence has at least 90% or at least 95% aa sequence identity to at least 80 or at least 90 contiguous aas of a DPB R 1 or J2 domain sequence of DPB1*01:01, DPB1*02:01, DPB1*03:01, DPB1*04:01, DPB1*06:01, DPB1*09:01, DPB1*11:01, DPB1*13:01, DPB1*35:01, DPB1*71:01, DPB1*104:01, or DPB1*141:01; and/or
the MHC Class II α chain polypeptide sequence has at least 90% or at least 95% aa sequence identity to at least 70 or at least 80 contiguous aas of the DPA α1 or α2 domain sequence of DPA1*01:03 or DPA1*02:01.

12-14. (canceled)

15. The CIIC of claim 1, further comprising at least one immunomodulatory polypeptide (“MOD”), or two or more independently selected MODs optionally placed in tandem.

16. The CIIC of claim 15, wherein the at least one MOD or the two or more independently selected MODs comprise human MOD sequences selected from the group consisting of: IL-1, IL-2, IL-4, IL-6, IL-7, IL-10, IL-12, IL-15, IL-17, IL-21, IL-23, CD7, CD30L, CD40, CD70, CD80 (B7-1), CD83, CD86 (B7-2), HVEM (CD270), ILT3, ILT4, Fas ligand (FasL), ICAM, ICOS-L, JAG1 (CD339), lymphotoxin beta receptor, 3/TR6, OX40L (CD252), PD-L1, PD-L2, TGF-β1 which may be masked, TGF-β2 which may be masked, TGF-β3 which may be masked, 4-1BBL polypeptide sequences, and variants of any thereof.

17. (canceled)

18. The CIIC of claim 15, comprising at least one IL-2 or variant IL-2 MOD, wherein the variant IL-2 optionally comprises an alanine or threonine substitution of one or both of F42 and H16.

19. The CIIC of claim 15, wherein the peptide epitope is an epitope of: an autoantigen, cancer-associated antigen, grafted tissue, infectious agent, or allergen that is from 4 aas to about 25 aas or about 8 aas to about 20 aas.

20. The CIIC of claim 19, wherein the epitope is an epitope of:
A) an autoantigen associated with an autoimmune disease selected from the group consisting of: celiac disease, TlD, Addison's disease, alopecia areata, ankylosing spondylitis, autoimmune encephalomyelitis, autoimmune hemolytic anemia, autoimmune hepatitis, autoimmune-associated infertility, autoimmune thrombocytopenic purpura, bullous pemphigoid, Crohn's disease, Goodpasture's syndrome, glomerulonephritis, Grave's disease, Hashimoto's thyroiditis, mixed connective tissue disease, multiple sclerosis, myasthenia gravis, pemphigus, pernicious anemia, polymyositis, psoriasis, psoriatic arthritis, rheumatoid arthritis, scleroderma, Sjagren's syndrome, systemic lupus erythematosus, vasculitis, or vitiligo; or
B) an autoantigen, selected from the group consisting of tissue transglutaminases, glutens, gliadins, secalins, hordeins, avenins, and glutenins, preproinsulin, proinsulin, insulin, insulin B chain, insulin A chain, 65 kDa isoform of glutamic acid decarboxylase (GAD65), 67 kDa isoform of glutamic acid decarboxylase (GAD67), tyrosine phosphatase (IA-2), heat-shock protein HSP65, islet-specific glucose-6-phosphatase catalytic subunit related protein (IGRP), islet antigen 2 (IA2), and zinc transporter (ZnT8).

21. (canceled)

22. The CIIC of claim 19, comprising an interspecific or non-interspecific immunoglobulin scaffold sequence that forms a duplex CIIC structure comprising a first CIIC and a second CIIC; wherein the scaffold sequence of the first CIIC and second CIIC are immunoglobulin Fc (Ig Fc) scaffold sequences that are optionally linked by either one, two, or more interchain disulfide bonds between the scaffold sequence of the first CIIC and the scaffold sequence of the second CIIC; and
wherein the immunoglobulin Fc scaffold sequences of the first CIIC and second CIIC
optionally comprise one or more substitutions that reduce ADCC, ADCP, and/or CDC
relative to an otherwise identical duplex CIIC that does not bear the substitutions.

23. (canceled)

24. A pharmaceutical composition comprising one or more CIICs or duplex CIICs of claim 22.

25. A nucleic acid or recombinant expression vector comprising a nucleic acid sequence encoding one or more CIICs or duplex CIICs of claim 22.

26. A pharmaceutical composition comprising one or more nucleic acids or expression vectors of claim 25.

27. A method of treatment or prophylaxis of a patient or subject having a disease or condition comprising:
(i) administering to a patient or subject an effective amount of one or more CIICs or duplex CIICs of claim 22; or
(ii) contacting a cell or tissue, either in vitro or in vivo, with one or more CIICs or duplex CIICs of claim 22, and administering the cell, tissue, or progeny thereof to the patient or subject.

28. The method of claim 27, wherein the disease or condition is selected from the group consisting of: an autoimmune disease, GVHD, HGVD, an infection, a metabolic disorder, a cancer, or an allergy.

29. The method of claim 28, wherein the autoimmune disease is selected from the group consisting of: celiac disease, TlD, Addison's disease, alopecia areata, ankylosing spondylitis, autoimmune encephalomyelitis, autoimmune hemolytic anemia, autoimmune hepatitis, autoimmune-associated infertility, autoimmune thrombocytopenic purpura, bullous pemphigoid, Crohn's disease, Goodpasture's syndrome, glomerulonephritis, Grave's disease, Hashimoto's thyroiditis, autoimmune gastritis, inflammatory bowel diseases, irritable bowel disease or syndrome, mixed connective tissue disease, multiple sclerosis, myasthenia gravis (MG), pemphigus , pernicious anemia, polymyositis, psoriasis, psoriatic arthritis, rheumatoid arthritis, scleroderma, Sjagren's syndrome, systemic lupus erythematosus (SLE), vasculitis, and vitiligo.


SEQUENCE LISTING (500 sequences)

SEQ ID NO: 1 - protein, 352 aa, Homo sapiens
  REGION 1..352: note = Description of sequence: Homo sapiens IgA (partial sequence)
ASPTSPVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 2 - protein, 383 aa, Homo sapiens
  REGION 1..383: note = Description of sequence: Homo sapiens IgD (partial sequence)
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 3 - protein, 222 aa, Homo sapiens
  REGION 1..222: note = Description of sequence: Homo sapiens IgE (partial sequence)
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 4 - protein, 227 aa, Homo sapiens
  REGION 1..227: note = Description of sequence: WT Homo sapiens IgG1 (partial sequence)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 227 aa, Homo sapiens
  REGION 1..227: note = Description of sequence: Homo sapiens IgG1 (partial sequence) Fc
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 227 aa, Homo sapiens
  REGION 1..227: note = Description of sequence: Homo sapiens IgG1 (partial sequence) Fc Variant: L234F/L235E/P331S
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 227 aa, Homo sapiens
  REGION 1..227: note = Description of sequence: Homo sapiens IgG1 (partial sequence) Fc Variant: N297A
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 227 aa, Homo sapiens
  REGION 1..227: note = Description of sequence: Homo sapiens IgG1 (partial sequence) Fc Variant: L234A/L235A ("LALA")
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 325 aa, Homo sapiens
  REGION 1..325: note = Description of sequence: Homo sapiens IgG2 (partial sequence)
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 246 aa, Homo sapiens
  REGION 1..246: note = Description of sequence: Homo sapiens IgG3 (partial sequence)
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 327 aa, Homo sapiens
  REGION 1..327: note = Description of sequence: Homo sapiens IgG4 (partial sequence)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 223 aa, Homo sapiens
  REGION 1..223: note = Description of sequence: Homo sapiens IgG4 (partial sequence)
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 13 - protein, 453 aa, Homo sapiens
  REGION 1..453: note = Description of sequence: Homo sapiens IgM (partial sequence)
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 14 - protein, 112 aa, Homo sapiens
  REGION 1..112: note = Description of sequence: Ig CH1 domain
FTVRETASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 15 - protein, 159 aa, Homo sapiens
  REGION 1..159: note = Description of sequence: Homo sapiens Ig J-chain (amino acids 23-259)
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 16 - protein, 106 aa, Homo sapiens
  REGION 1..106: note = Description of sequence: Homo sapiens Ig Kappa chain constant region
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 106 aa, Homo sapiens
  REGION 1..106: note = Description of sequence: Homo sapiens Ig Lamba chain constant region
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 18 - protein, 229 aa, Homo sapiens
  REGION 1..229: note = Description of sequence: DRA*01:02
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLRKSNAAERRGPL

SEQ ID NO: 19 - protein, 198 aa, Homo sapiens
  REGION 1..198: note = Description of sequence: DRB1-1 (DRB1*01:01)
GDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSK

SEQ ID NO: 20 - protein, 198 aa, Homo sapiens
  REGION 1..198: note = Description of sequence: DRB1- (DRB*01:02)
GDTRPRFLWQLKFECHFFNGTERVRLLERCIYNQEESVRFDSDVGEYRAVTELGRPDAEYWNSQKDLLEQRRAAVDTYCRHNYGAVESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSK

SEQ ID NO: 21 - protein, 198 aa, Homo sapiens
  REGION 1..198: note = Description of sequence: DRB1- (DRB1*01:03)
GDTRPRFLWQLKFECHFFNGTERVRLLERCIYNQEESVRFDSDVGEYRAVTELGRPDAEYWNSQKDILEDERAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSK

SEQ ID NO: 22 - protein, 198 aa, Homo sapiens
  REGION 1..198: note = Description of sequence: DRB1-3 (DRB1*03:01 sp P01912.2
GDTRPRFLEYSTSECHFFNGTERVRYLDRYFHNQEENVRFDSDVGEFRAVTELGRPDAEYWNSQKDLLEQKRGRVDNYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 23 - protein, 198 aa, Homo sapiens
  REGION 1..198: note = Description of sequence: DRB1- (DRB1*03:02)
GDTRPRFLEYSTSECHFFNGTERVRFLERYFHNQEENVRFDSDVGEYRAVTELGRPDAEYWNSQKDLLEQKRGRVDNYCRHNYGVGESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 24 - protein, 198 aa, Homo sapiens
  REGION 1..198: note = Description of sequence: DRB1- (DRB1*03:04)
GDTRPRFLEYSTSECHFFNGTERVRYLDRYFHNQEESVRFDSDVGEFRAVTELGRPDAEYWNSQKDLLEQKRGRVDNYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 25 - protein, 198 aa, Homo sapiens
  REGION 1..198: note = Description of sequence: DRB1-4 (DRB1*04:01 sp P13760.1
GDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 26 - protein, 198 aa, Homo sapiens
  REGION 1..198: note = Description of sequence: DRB1*04:02
GDTRPRFLEQVKHECHFFNGTERVRFLDRYFYHQEEYVRFDSDVGEYRAVTELGRPDAEYWNSQKDILEDERAAVDTYCRHNYGVVESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 27 - protein, 198 aa, Homo sapiens
  REGION 1..198: note = Description of sequence: DRB1*04:03
GDTRPRFLEQVKHECHFFNGTERVRFLDRYFYHQEEYVRFDSDVGEYRAVTELGRPDAEYWNSQKDLLEQRRAEVDTYCRHNYGVVESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 28 - protein, 198 aa, Homo sapiens
  REGION 1..198: note = Description of sequence: DRB1*04:04
GDTRPRFLEQVKHECHFFNGTERVRFLDRYFYHQEEYVRFDSDVGEYRAVTELGRPDAEYWNSQKDLLEQRRAAVDTYCRHNYGVVESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 29 - protein, 198 aa, Homo sapiens
  REGION 1..198: note = Description of sequence: DRB1*04:05
GDTRPRFLEQVKHECHFFNGTERVRFLDRYFYHQEEYVRFDSDVGEYRAVTELGRPSAEYWNSQKDLLEQRRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 30 - protein, 198 aa, Homo sapiens
  REGION 1..198: note = Description of sequence: DRB1*04:06
GDTRPRFLEQVKHECHFFNGTERVRFLDRYFYHQEESVRFDSDVGEYRAVTELGRPDAEYWNSQKDLLEQRRAEVDTYCRHNYGVVESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 31 - protein, 198 aa, Homo sapiens
  REGION 1..198: note = Description of sequence: DRB1*04:08
GDTRPRFLEQVKHECHFFNGTERVRFLDRYFYHQEEYVRFDSDVGEYRAVTELGRPDAEYWNSQKDLLEQRRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 32 - protein, 198 aa, Homo sapiens
  REGION 1..198: note = Description of sequence: DRB1-7 (DRB1*07:01 sp P13761.1
GDTQPRFLWQGKYKCHFFNGTERVQFLERLFYNQEEFVRFDSDVGEYRAVTELGRPVAESWNSQKDILEDRRGQVDTVCRHNYGVGESFTVQRRVHPEVTVYPAKTQPLQHHNLLVCSVSGFYPGSIEVRWFRNGQEEKAGVVSTGLIQNGDWTFQTLVMLETVPRSGEVYTCQVEHPSVMSPLTVEWRARSESAQSK

SEQ ID NO: 33 - protein, 198 aa, Homo sapiens
  REGION 1..198: note = Description of sequence: DRB1-8 (DRB1*08:01 sp Q30134.2
GDTRPRFLEYSTGECYFFNGTERVRFLDRYFYNQEEYVRFDSDVGEYRAVTELGRPSAEYWNSQKDFLEDRRALVDTYCRHNYGVGESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWSARSESAQSK

SEQ ID NO: 34 - protein, 198 aa, Homo sapiens
  REGION 1..198: note = Description of sequence: DRB1*08:02
GDTRPRFLEYSTGECYFFNGTERVRFLDRYFYNQEEYVRFDSDVGEYRAVTELGRPDAEYWNSQKDFLEDRRALVDTYCRHNYGVGESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWSARSESAQSK

SEQ ID NO: 35 - protein, 198 aa, Homo sapiens
  REGION 1..198: note = Description of sequence: DRB1*08:03
GDTRPRFLEYSTGECYFFNGTERVRFLDRYFYNQEEYVRFDSDVGEYRAVTELGRPSAEYWNSQKDILEDRRALVDTYCRHNYGVGESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWSARSESAQSK

SEQ ID NO: 36 - protein, 198 aa, Homo sapiens
  REGION 1..198: note = Description of sequence: DRB1-9 (DRB1*09:01 sp Q9TQE0.1
GDTQPRFLKQDKFECHFFNGTERVRYLHRGIYNQEENVRFDSDVGEYRAVTELGRPVAESWNSQKDFLERRRAEVDTVCRHNYGVGESFTVQRRVHPEVTVYPAKTQPLQHHNLLVCSVSGFYPGSIEVRWFRNGQEEKAGVVSTGLIQNGDWTFQTLVMLETVPRSGEVYTCQVEHPSVMSPLTVEWRARSESAQSK

SEQ ID NO: 37 - protein, 198 aa, Homo sapiens
  REGION 1..198: note = Description of sequence: DRB1-10 (DRB1*10:01 sp Q30167.2
GDTRPRFLEEVKFECHFFNGTERVRLLERRVHNQEEYARYDSDVGEYRAVTELGRPDAEYWNSQKDLLERRRAAVDTYCRHNYGVGESFTVQRRVQPKVTVYPSKTQPLQHHNLLVCSVNGFYPGSIEVRWFRNGQEEKTGVVSTGLIQNGDWTFQTLVMLETVPQSGEVYTCQVEHPSVMSPLTVEWRARSESAQSK

SEQ ID NO: 38 - protein, 198 aa, Homo sapiens
  REGION 1..198: note = Description of sequence: DRB1-11 DRB1*11:01 sp P20039.1
GDTRPRFLEYSTSECHFFNGTERVRFLDRYFYNQEEYVRFDSDVGEFRAVTELGRPDEEYWNSQKDFLEDRRAAVDTYCRHNYGVGESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 39 - protein, 198 aa, Homo sapiens
  REGION 1..198: note = Description of sequence: DRB1*11:03
GDTRPRFLEYSTSECHFFNGTERVRFLDRYFYNQEEYVRFDSDVGEFRAVTELGRPDEEYWNSQKDFLEDERAAVDTYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 40 - protein, 198 aa, Homo sapiens
  REGION 1..198: note = Description of sequence: DRB1*11:04
GDTRPRFLEYSTSECHFFNGTERVRFLDRYFYNQEEYVRFDSDVGEFRAVTELGRPDEEYWNSQKDFLEDRRAAVDTYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 41 - protein, 198 aa, Homo sapiens
  REGION 1..198: note = Description of sequence: DRB1-12 (DRB1*12:01 sp Q95IE3.1
GDTRPRFLEYSTGECYFFNGTERVRLLERHFHNQEELLRFDSDVGEFRAVTELGRPVAESWNSQKDILEDRRAAVDTYCRHNYGAVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 42 - protein, 198 aa, Homo sapiens
  REGION 1..198: note = Description of sequence: DRB1-13 (DRB1*13:01 sp Q5Y7A7.1
GDTRPRFLEYSTSECHFFNGTERVRFLDRYFHNQEENVRFDSDVGEFRAVTELGRPDAEYWNSQKDILEDERAAVDTYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 43 - protein, 198 aa, Homo sapiens
  REGION 1..198: note = Description of sequence: DRB1*13:03
GDTRPRFLEYSTSECHFFNGTERVRFLDRYFYNQEEYVRFDSDVGEYRAVTELGRPSAEYWNSQKDILEDKRAAVDTYCRHNYGVGESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 44 - protein, 198 aa, Homo sapiens
  REGION 1..198: note = Description of sequence: DRB1-14 (DRB1*14:01 sp Q9GIY3.1
GDTRPRFLEYSTSECHFFNGTERVRFLDRYFHNQEEFVRFDSDVGEYRAVTELGRPAAEHWNSQKDLLERRRAEVDTYCRHNYGVVESFTVQRRVHPKVTVYPSKTQPLQHYNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 45 - protein, 198 aa, Homo sapiens
  REGION 1..198: note = Description of sequence: DRB1*14:02
GDTRPRFLEYSTSECHFFNGTERVRFLERYFHNQEENVRFDSDVGEYRAVTELGRPDAEYWNSQKDLLEQRRAAVDTYCRHNYGVGESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 46 - protein, 198 aa, Homo sapiens
  REGION 1..198: note = Description of sequence: DRB1*14:05
GDTRPRFLEYSTSECQFFNGTERVRFLDRYFHNQEEFVRFDSDVGEYRAVTELGRPDAEYWNSQKDLLERRRAEVDTYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 47 - protein, 198 aa, Homo sapiens
  REGION 1..198: note = Description of sequence: DRB1*14:06
GDTRPRFLEYSTSECHFFNGTERVRFLERYFHNQEENVRFDSDVGEYRAVTELGRPDAEYWNSQKDLLEQRRAAVDTYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 48 - protein, 198 aa, Homo sapiens
  REGION 1..198: note = Description of sequence: DRB1-15 (DRB1*15:01 sp P01911
GDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSK

SEQ ID NO: 49 - protein, 198 aa, Homo sapiens
  REGION 1..198: note = Description of sequence: DRB1*15:02
GDTRPRFLWQPKRECHFFNGTERVRFLDRYFYNQEESVRFDSDVGEFRAVTELGRPDAEYWNSQKDILEQARAAVDTYCRHNYGVGESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSK

SEQ ID NO: 50 - protein, 198 aa, Homo sapiens
  REGION 1..198: note = Description of sequence: DRB1*15:03
GDTRPRFLWQPKRECHFFNGTERVRFLDRHFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSK

SEQ ID NO: 51 - protein, 198 aa, Homo sapiens
  REGION 1..198: note = Description of sequence: DRB1*15:04
GDTRPRFLWQPKRECHFFNGTERVRFLDRYFYNQEESVRFDSDVGEFRAVTELGRPDAEYWNSQKDFLEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSK

SEQ ID NO: 52 - protein, 198 aa, Homo sapiens; identical to SEQ ID NO: 48
  REGION 1..198: note = Description of sequence: DRB1*15:05

SEQ ID NO: 53 - protein, 198 aa, Homo sapiens; identical to SEQ ID NO: 48
  REGION 1..198: note = Description of sequence: DRB1*15:06

SEQ ID NO: 54 - protein, 198 aa, Homo sapiens
  REGION 1..198: note = Description of sequence: DRB1*15:07
GDTRPRFLWQPKRECHFFNGTERVRFLDRYFYNQEESVRFDSDVGEYRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSK

SEQ ID NO: 55 - protein, 198 aa, Homo sapiens
  REGION 1..198: note = Description of sequence: DRB1*16:01 sp Q29974.1
GDTRPRFLWQPKRECHFFNGTERVRFLDRYFYNQEESVRFDSDVGEYRAVTELGRPDAEYWNSQKDFLEDRRAAVDTYCRHNYGVGESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSK

SEQ ID NO: 56 - protein, 198 aa, Homo sapiens
  REGION 1..198: note = Description of sequence: DRB3*01:01
GDTRPRFLELRKSECHFFNGTERVRYLDRYFHNQEEFLRFDSDVGEYRAVTELGRPVAESWNSQKDLLEQKRGRVDNYCRHNYGVGESFTVQRRVHPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSK

SEQ ID NO: 57 - protein, 198 aa, Homo sapiens
  REGION 1..198: note = Description of sequence: DRB3*02:01
GDTRPRFLELLKSECHFFNGTERVRFLERHFHNQEEYARFDSDVGEYRAVRELGRPDAEYWNSQKDLLEQKRGQVDNYCRHNYGVVESFTVQRRVHPQVTVYPAKTQPLQHHNLLVCSVSGFYPGSIEVRWFRNGQEEKAGVVSTGLIQNGDWTFQTLVMLETFPRSGEVYTCQVEHPSVTSPLTVEWSARSESAQSK

SEQ ID NO: 58 - protein, 198 aa, Homo sapiens
  REGION 1..198: note = Description of sequence: DRB3*03:01
GDTRPRFLELLKSECHFFNGTERVRFLERYFHNQEEFVRFDSDVGEYRAVTELGRPVAESWNSQKDLLEQKRGQVDNYCRHNYGVVESFTVQRRVHPQVTVYPAKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 59 - protein, 198 aa, Homo sapiens
  REGION 1..198: note = Description of sequence: DRB4*01:01
GDTQPRFLEQAKCECHFLNGTERVWNLIRYIYNQEEYARYNSDLGEYQAVTELGRPDAEYWNSQKDLLERRRAEVDTYCRYNYGVVESFTVQRRVQPKVTVYPSKTQPLQHHNLLVCSVNGFYPGSIEVRWFRNSQEEKAGVVSTGLIQNGDWTFQTLVMLETVPRSGEVYTCQVEHPSMMSPLTVQWSARSESAQSK

SEQ ID NO: 60 - protein, 198 aa, Homo sapiens
  REGION 1..198: note = Description of sequence: DRB4*01:03
GDTRPRFLQQDKYECHFFNGTERVRFLHRDIYNQEEDLRFDSDVGEYRAVTELGRPDAEYWNSQKDFLEDRRAAVDTYCRHNYGVGESFTVQRRVEPKVTVYPARTQTLQHHNLLVCSVNGFYPGSIEVRWFRNSQEEKAGVVSTGLIQNGDWTFQTLVMLETVPRSGEVYTCQVEHPSVTSPLTVEWRAQSESAQSK

SEQ ID NO: 61 - protein, 198 aa, Homo sapiens; identical to SEQ ID NO: 60
  REGION 1..198: note = Description of sequence: MHC Class II DRB5 beta chain

SEQ ID NO: 62 - protein, 232 aa, Homo sapiens
  REGION 1..232: note = Description of sequence: DPA1*01:03
AGAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTETVLCALGLVLGLVGIIVGTVLIIKSLRSGHDPRAQGTL

SEQ ID NO: 63 - protein, 232 aa, Homo sapiens
  REGION 1..232: note = Description of sequence: DPA1*02:01
AGAIKADHVSTYAAFVQTHRPTGEFMFEFDEDEQFYVDLDKKETVWHLEEFGRAFSFEAQGGLANIAILNNNLNTLIQRSNHTQAANDPPEVTVFPKEPVELGQPNTLICHIDRFFPPVLNVTWLCNGEPVTEGVAESLFLPRTDYSFHKFHYLTFVPSAEDVYDCRVEHWGLDQPLLKHWEAQEPIQMPETTETVLCALGLVLGLVGIIVGTVLIIKSLRSGHDPRAQGPL

SEQ ID NO: 64 - protein, 196 aa, Homo sapiens
  REGION 1..196: note = Description of sequence: DPB1*01:01 IMGT/HLA Acc No: HLA00510
RATPENYVYQGRQECYAFNGTQRFLERYIYNREEYARFDSDVGEFRAVTELGRPAAEYWNSQKDILEEKRAVPDRVCRHNYELDEAVTLQRRVQPKVNVSPSKKGPLQHHNLLVCHVTDFYPGSIQVRWFLNGQEETAGVVSTNLIRNGDWTFQILVMLEMTPQQGDVYICQVEHTSLDSPVTVEWKAQSDSAQSK

SEQ ID NO: 65 - protein, 196 aa, Homo sapiens
  REGION 1..196: note = Description of sequence: DPB1*02:01 IMGT/HLA Acc No: HLA00517
RATPENYLFQGRQECYAFNGTQRFLERYIYNREEFVRFDSDVGEFRAVTELGRPDEEYWNSQKDILEEERAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEWKAQSDSARSK

SEQ ID NO: 66 - protein, 196 aa, Homo sapiens
  REGION 1..196: note = Description of sequence: DPB1*03:01 IMGT/HLA Acc No: HLA00520
RATPENYVYQLRQECYAFNGTQRFLERYIYNREEFVRFDSDVGEFRAVTELGRPDEDYWNSQKDLLEEKRAVPDRVCRHNYELDEAVTLQRRVQPKVNVSPSKKGPLQHHNLLVCHVTDFYPGSIQVRWFLNGQEETAGVVSTNLIRNGDWTFQILVMLEMTPQQGDVYICQVEHTSLDSPVTVEWKAQSDSARSK

SEQ ID NO: 67 - protein, 196 aa, Homo sapiens
  REGION 1..196: note = Description of sequence: DPB1*04:01 IMGT/HLA Acc No: HLA00521
RATPENYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEWKAQSDSARSK

SEQ ID NO: 68 - protein, 196 aa, Homo sapiens
  REGION 1..196: note = Description of sequence: DPB1*04:02 IMGT/HLA Acc No: HLA00522
RATPENYLFQGRQECYAFNGTQRFLERYIYNREEFVRFDSDVGEFRAVTELGRPDEEYWNSQKDILEEKRAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSMDSPVTVEWKAQSDSARSK

SEQ ID NO: 69 - protein, 196 aa, Homo sapiens
  REGION 1..196: note = Description of sequence: DPB1*06:01 IMGT/HLA Acc No: HLA00524
RATPENYVYQLRQECYAFNGTQRFLERYIYNREEFVRFDSDVGEFRAVTELGRPDEDYWNSQKDLLEEERAVPDRMCRHNYELDEAVTLQRRVQPKVNVSPSKKGPLQHHNLLVCHVTDFYPGSIQVRWFLNGQEETAGVVSTNLIRNGDWTFQILVMLEMTPQQGDVYICQVEHTSLDSPVTVEWKAQSDSARSK

SEQ ID NO: 70 - protein, 196 aa, Homo sapiens
  REGION 1..196: note = Description of sequence: DPB1*09:01
RATPENYVHQLRQECYAFNGTQRFLERYIYNREEFVRFDSDVGEFRAVTELGRPDEDYWNSQKDILEEERAVPDRVCRHNYELDEAVTLQRRVQPKVNVSPSKKGPLQHHNLLVCHVTDFYPGSIQVRWFLNGQEETAGVVSTNLIRNGDWTFQILVMLEMTPQQGDVYICQVEHTSLDSPVTVEWKAQSDSARSK

SEQ ID NO: 71 - protein, 196 aa, Homo sapiens
  REGION 1..196: note = Description of sequence: DPB1*11:01 IMGT/HLA Acc No: HLA00528
RATPENYVYQLRQECYAFNGTQRFLERYIYNRQEYARFDSDVGEFRAVTELGRPAAEYWNSQKDLLEERRAVPDRMCRHNYELDEAVTLQRRVQPKVNVSPSKKGPLQHHNLLVCHVTDFYPGSIQVRWFLNGQEETAGVVSTNLIRNGDWTFQILVMLEMTPQQGDVYICQVEHTSLDSPVTVEWKAQSDSARSK

SEQ ID NO: 72 - protein, 196 aa, Homo sapiens
  REGION 1..196: note = Description of sequence: DPB1*13:01
RATPENYVYQLRQECYAFNGTQRFLERYIYNREEYARFDSDVGEFRAVTELGRPAAEYWNSQKDILEEERAVPDRICRHNYELDEAVTLQRRVQPKVNVSPSKKGPLQHHNLLVCHVTDFYPGSIQVRWFLNGQEETAGVVSTNLIRNGDWTFQILVMLEMTPQQGDVYICQVEHTSLDSPVTVEWKAQSDSARSK

SEQ ID NO: 73 - protein, 196 aa, Homo sapiens
  REGION 1..196: note = Description of sequence: DPB1*35:01
RATPENYVHQLRQECYAFNGTQRFLERYIYNREEFVRFDSDVGEFRAVTELGRPDEDYWNSQKDILEEKRAVPDRVCRHNYELDEAVTLQRRVQPKVNVSPSKKGPLQHHNLLVCHVTDFYPGSIQVRWFLNGQEETAGVVSTNLIRNGDWTFQILVMLEMTPQQGDVYICQVEHTSLDSPVTVEWKAQSDSARSK

SEQ ID NO: 74 - protein, 196 aa, Homo sapiens
  REGION 1..196: note = Description of sequence: DPB1*71:01 IMGT/HLA Acc No:HLA00590
RATPENYLFQGRQECYAFNGTQRFLERYIYNREEFVRFDSDVGEFRAVTELGRPAAEYWNSQKDILEEERAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEWKAQSDSARSK

SEQ ID NO: 75 - protein, 196 aa, Homo sapiens; identical to SEQ ID NO: 66
  REGION 1..196: note = Description of sequence: DPB1*104:01 Beta chain aa sequence IMGT/HLA Acc No: HLA02046

SEQ ID NO: 76 - protein, 196 aa, Homo sapiens
  REGION 1..196: note = Description of sequence: DPB1*141:01 beta chain aa sequence
RATPENYLFQGRQECYAFNGTQRFLERYIYNREEFVRFDSDVGEFRAVTELGRPDEEYWNSQKDILEEERAVPDRMCRHNYELGGPMTLQRRVQPRVNVSPSKKGPLQHHNLLVCHVTDFYPGSIQVQWFLNGQEETAGVVSTNLIRNGDWTFQILVMLEMTPQQGDVYTCQVEHTSLDSPVTVEWKAQSDSARSK

SEQ ID NO: 77 - protein, 232 aa, Homo sapiens
  REGION 1..232: note = Description of sequence: DQA1*01:01
EDIVADHVASCGVNLYQFYGPSGQYTHEFDGDEEFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTETVVCALGLSVGLVGIVVGTVFIIQGLRSVGASRHQGPL

SEQ ID NO: 78 - protein, 232 aa, Homo sapiens
  REGION 1..232: note = Description of sequence: DQA1*01:02
EDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTETVVCALGLSVGLMGIVVGTVFIIQGLRSVGASRHQGPL

SEQ ID NO: 79 - protein, 232 aa, Homo sapiens
  REGION 1..232: note = Description of sequence: DQA1*01:03
EDIVADHVASCGVNLYQFYGPSGQFTHEFDGDEQFYVDLEKKETAWRWPEFSKFGGFDPQGALRNMAVAKHNLNIMIKRYNSTAATNEVPEVTVFSKSPVTLGQPNTLICLVDNIFPPVVNITWLSNGHAVTEGVSETSFLSKSDHSFFKISYLTFLPSADEIYDCKVEHWGLDQPLLKHWEPEIPAPMSELTETVVCALGLSVGLVGIVVGTVFIIQGLRSVGASRHQGPL

SEQ ID NO: 80 - protein, 232 aa, Homo sapiens
  REGION 1..232: note = Description of sequence: DQA1*01:04
EGIVADHVASCGVNLYQFYGPSGQYTHEFDGDEEFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTETVVCTLGLSVGLVGIVVGTVFIIQGLRSVGASRHQGPL

SEQ ID NO: 81 - protein, 231 aa, Homo sapiens
  REGION 1..231: note = Description of sequence: DQA1*02:01
EDIVADHVASYGVNLYQSYGPSGQFTHEFDGDEEFYVDLERKETVWKLPLFHRLRFDPQFALTNIAVLKHNLNILIKRSNSTAATNEVPEVTVFSKSPVTLGQPNTLICLVDNIFPPVVNITWLSNGHSVTEGVSETSFLSKSDHSFFKISYLTFLPSADEIYDCKVEHWGLDEPLLKHWEPEIPAPMSELTETVVCALGLSVGLVGIVVGTVLIIRGLRSVGASRHQGPL

SEQ ID NO: 82 - protein, 232 aa, Homo sapiens
  REGION 1..232: note = Description of sequence: DQA1*03:01:
EDIVADHVASYGVNLYQSYGPSGQYSHEFDGDEEFYVDLERKETVWQLPLFRRFRRFDPQFALTNIAVLKHNLNIVIKRSNSTAATNEVPEVTVFSKSPVTLGQPNTLICLVDNIFPPVVNITWLSNGHSVTEGVSETSFLSKSDHSFFKISYLTFLPSADEIYDCKVEHWGLDEPLLKHWEPEIPTPMSELTETVVCALGLSVGLVGIVVGTVLIIRGLRSVGASRHQGPL

SEQ ID NO: 83 - protein, 232 aa, Homo sapiens
  REGION 1..232: note = Description of sequence: DQA1*03:02
EDIVADHVASYGVNLYQSYGPSGQYSHEFDGDEEFYVDLERKETVWQLPLFRRFRRFDPQFALTNIAVLKHNLNIVIKRSNSTAATNEVPEVTVFSKSPVTLGQPNTLICLVDNIFPPVVNITWLSNGHSVTEGVSETSFLSKSDHSFFKISYLTFLPSDDEIYDCKVEHWGLDEPLLKHWEPEIPTPMSELTETVVCALGLSVGLVGIVVGTVLIIRGLRSVGASRHQGPL

SEQ ID NO: 84 - protein, 231 aa, Homo sapiens
  REGION 1..231: note = Description of sequence: DQA1*04:01
EDIVADHVASYGVNLYQSYGPSGQYTHEFDGDEQFYVDLGRKETVWCLPVLRQFRFDPQFALTNIAVTKHNLNILIKRSNSTAATNEVPEVTVFSKSPVTLGQPNTLICLVDNIFPPVVNITWLSNGHSVTEGVSETSFLSKSDHSFFKISYLTFLPSADEIYDCKVEHWGLDEPLLKHWEPEIPAPMSELTETVVCALGLSVGLVGIVVGTVFIIRGLRSVGASRHQGPL

SEQ ID NO: 85 - protein, 231 aa, Homo sapiens
  REGION 1..231: note = Description of sequence: DQA1*05:01 UniProtKB/Swiss-Prot: P01909
EDIVADHVASYGVNLYQSYGPSGQYTHEFDGDEQFYVDLGRKETVWCLPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTETVVCALGLSVGLVGIVVGTVFIIRGLRSVGASRHQGPL

SEQ ID NO: 86 - protein, 231 aa, Homo sapiens; identical to SEQ ID NO: 85
  REGION 1..231: note = Description of sequence: DQA1*05:05

SEQ ID NO: 87 - protein, 231 aa, Homo sapiens
  REGION 1..231: note = Description of sequence: DQA1*06:01
EDIVADHVASYGVNLYQSYGPSGQFTHEFDGDEQFYVDLGRKETVWCLPVLRQFRFDPQFALTNIAVTKHNLNILIKRSNSTAATNEVPEVTVFSKSPVTLGQPNTLICLVDNIFPPVVNITWLSNGHSVTEGVSETSFLSKSDHSFFKISYLTFLPSADEIYDCKVEHWGLDEPLLKHWEPEIPAPMSELTETVVCALGLSVGLVGIVVGTVFIIRGLRSVGASRHQGPL

SEQ ID NO: 88 - protein, 232 aa, Homo sapiens
  REGION 1..232: note = Description of sequence: DQA2*01:01
EDIVADHVASYGVNFYQSHGPSGQYTHEFDGDEEFYVDLETKETVWQLPMFSKFISFDPQSALRNMAVGKHTLEFMMRQSNSTAATNEVPEVTVFSKFPVTLGQPNTLICLVDNIFPPVVNITWLSNGHSVTEGVSETSFLSKSDHSFFKISYLTFLPSADEIYDCKVEHWGLDEPLLKHWEPEIPAPMSELTETLVCALGLSVGLMGIVVGTVFIIQGLRSVGASRHQGLL

SEQ ID NO: 89 - protein, 198 aa, Homo sapiens
  REGION 1..198: note = Description of sequence: DQB1*02:01 NP_001230891.1
RDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSK

SEQ ID NO: 90 - protein, 198 aa, Homo sapiens
  REGION 1..198: note = Description of sequence: DQB1*02:02 6PX6_B
RDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNGQEETAGVVSTPLIRNGDWTFQILVMLEMTPQRGDVYTCHVEHPSLQSPITVEWRAQSESAQSK

SEQ ID NO: 91 - protein, 198 aa, Homo sapiens
  REGION 1..198: note = Description of sequence: DQB1*03:01
RDSPEDFVYQFKAMCYFTNGTERVRYVTRYIYNREEYARFDSDVEVYRAVTPLGPPDAEYWNSQKEVLERTRAELDTVCRHNYQLELRTTLQRRVEPTVTISPSRTEALNHHNLLVCSVTDFYPAQIKVRWFRNDQEETTGVVSTPLIRNGDWTFQILVMLEMTPQHGDVYTCHVEHPSLQNPITVEWRAQSESAQSK

SEQ ID NO: 92 - protein, 198 aa, Homo sapiens
  REGION 1..198: note = Description of sequence: DQB1*03:02
RDSPEDFVYQFKGMCYFTNGTERVRLVTRYIYNREEYARFDSDVGVYRAVTPLGPPAAEYWNSQKEVLERTRAELDTVCRHNYQLELRTTLQRRVEPTVTISPSRTEALNHHNLLVCSVTDFYPAQIKVRWFRNDQEETTGVVSTPLIRNGDWTFQILVMLEMTPQRGDVYTCHVEHPSLQNPIIVEWRAQSESAQSK

SEQ ID NO: 93 - protein, 198 aa, Homo sapiens
  REGION 1..198: note = Description of sequence: DQB1*03:03
RDSPEDFVYQFKGMCYFTNGTERVRLVTRYIYNREEYARFDSDVGVYRAVTPLGPPDAEYWNSQKEVLERTRAELDTVCRHNYQLELRTTLQRRVEPTVTISPSRTEALNHHNLLVCSVTDFYPAQIKVRWFRNDQEETTGVVSTPLIRNGDWTFQILVMLEMTPQRGDVYTCHVEHPSLQNPIIVEWRAQSESAQSK

SEQ ID NO: 94 - protein, 198 aa, Homo sapiens
  REGION 1..198: note = Description of sequence: DQB1*03:04
RDSPEDFVYQFKAMCYFTNGTERVRYVTRYIYNREEYARFDSDVEVYRAVTPLGPPAAEYWNSQKEVLERTRAELDTVCRHNYQLELRTTLQRRVEPTVTISPSRTEALNHHNLLVCSVTDFYPAQIKVRWFRNDQEETTGVVSTPLIRNGDWTFQILVMLEMTPQHGDVYTCHVEHPSLQNPITVEWRAQSESAQSK

SEQ ID NO: 95 - protein, 198 aa, Homo sapiens
  REGION 1..198: note = Description of sequence: DQB1*04:01
RDSPEDFVFQFKGMCYFTNGTELVRGVTRYIYNREEYARFDSDVGVYRAVTPLGRLDAEYWNSQKDILEEDRASVDTVCRHNYQLELRTTLQRRVEPTVTISPSRTEALNHHNLLVCSVTDFYPAQIKVRWFRNDQEETTGVVSTPLIRNGDWTFQILVMLEMTPQRGDVYTCHVEHPSLQNPIIVEWRAQSESAQSK

SEQ ID NO: 96 - protein, 198 aa, Homo sapiens
  REGION 1..198: note = Description of sequence: DQB1*04:02
RDSPEDFVFQFKGMCYFTNGTERVRGVTRYIYNREEYARFDSDVGVYRAVTPLGRLDAEYWNSQKDILEEDRASVDTVCRHNYQLELRTTLQRRVEPTVTISPSRTEALNHHNLLVCSVTDFYPAQIKVRWFRNDQEETTGVVSTPLIRNGDWTFQILVMLEMTPQRGDVYTCHVEHPSLQNPIIVEWRAQSESAQSK

SEQ ID NO: 97 - protein, 198 aa, Homo sapiens
  REGION 1..198: note = Description of sequence: DQB1*05:01
RDSPEDFVYQFKGLCYFTNGTERVRGVTRHIYNREEYVRFDSDVGVYRAVTPQGRPVAEYWNSQKEVLEGARASVDRVCRHNYEVAYRGILQRRVEPTVTISPSRTEALNHHNLLICSVTDFYPSQIKVRWFRNDQEETAGVVSTPLIRNGDWTFQILVMLEMTPQRGDVYTCHVEHPSLQSPITVEWRAQSESAQSK

SEQ ID NO: 98 - protein, 198 aa, Homo sapiens
  REGION 1..198: note = Description of sequence: DQB1*06:01
RDPPEDFVLQFKAMCYFTNGTERVRYVTRYIYNREEDVRFDSDVGVYRAVTPQGRPDAEYWNSQKDILERTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQHGDVYTCHVEHPSLQSPITVEWRAQSESAQNK

SEQ ID NO: 99 - protein, 198 aa, Homo sapiens
  REGION 1..198: note = Description of sequence: DQB1*06:02
RDSPEDFVFQFKGMCYFTNGTERVRLVTRYIYNREEYARFDSDVGVYRAVTPQGRPDAEYWNSQKEVLEGTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQRGDVYTCHVEHPSLQSPITVEWRAQSESAQSK

SEQ ID NO: 100 - protein, 197 aa, Homo sapiens
  REGION 1..197: note = Description of sequence: DQB2 Isoform I (DQB2-ISO-1)
RDFPKDFLVQFKGMCYFTNGTERVRGVARYIYNREEYGRFDSDVGEFQAVTELGRSIEDWNNYKDFLEQERAAVDKVCRHNYEAELRTTLQRQVEPTVTISPSRTEALNHHNLLVCSVTDFYPAQIKVRWFRNDQEETAGVVSTSLIRNGDWTFQILVMLEITPQRGDIYTCQVEHPSLQSPITVEWRAQSESAQSK

SEQ ID NO: 101 - protein, 197 aa, Homo sapiens
  REGION 1..197: note = Description of sequence: DQB2 Isoform 2
RDFPKDFLVQFKGMCYFTNGTERVRGVARYIYNREEYGRFDSDVGEFQAVTELGRSIEDWNNYKDFLEQERAAVDKVCRHNYEAELRTTLQRQVEPTVTISPSRTEALNHHNLLVCSVTDFYPAQIKVRWFRNDQEETAGVVSTSLIRNGDWTFQILVMLEITPQRGDIYTCQVEHPSLQSPITVEWRPRGPPPAGL

SEQ ID NO: 102 - protein, 178 aa, Homo sapiens
  REGION 1..178: note = Description of sequence: DRA*01:02
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHW

SEQ ID NO: 103 - protein, 181 aa, Homo sapiens
  REGION 1..181: note = Description of sequence: DPA1*01:03
AGAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHW

SEQ ID NO: 104 - protein, 5 aa, Homo sapiens
  REGION 1..5: note = Description of sequence: DRB1 alleles
QRRAA

SEQ ID NO: 105 - protein, 15 aa, Homo sapiens
  REGION 1..15: note = Description of sequence: myelin basic protein peptide
ENPVVHFFKNIVTPR

SEQ ID NO: 106 - protein, 15 aa, Homo sapiens
  REGION 1..15: note = Description of sequence: Alpha-synuclein
  MOD_RES 12: note = S denotes phosphoserine
GKTKEGVLYVGSKTK

SEQ ID NO: 107 - protein, 15 aa, Homo sapiens
  REGION 1..15: note = Description of sequence: Alpha-synuclein
  MOD_RES 11: note = S denotes phosphoserine
KTKEGVLYVGSKTKE

SEQ ID NO: 108 - protein, 15 aa, Homo sapiens
  REGION 1..15: note = Description of sequence: Alpha-synuclein
  MOD_RES 14: note = S denotes phosphoserine
MPVDPDNEAYEMPSE

SEQ ID NO: 109 - protein, 15 aa, Homo sapiens
  REGION 1..15: note = Description of sequence: Alpha-synuclein
  MOD_RES 9: note = S denotes phosphoserine
DNEAYEMPSEEGYQD

SEQ ID NO: 110 - protein, 14 aa, Homo sapiens
  REGION 1..14: note = Description of sequence: Alpha-synuclein
  MOD_RES 4: note = S denotes phosphoserine
EMPSEEGYQDYEPE

SEQ ID NO: 111 - protein, 12 aa, Homo sapiens
  REGION 1..12: note = Description of sequence: Alpha-synuclein
SEEGYQDYEPEA

SEQ ID NO: 112 - protein, 5 aa, Homo sapiens
  REGION 1..5: note = Description of sequence: Class II HLA motif
QKRAA

SEQ ID NO: 113 - protein, 5 aa, Homo sapiens
  REGION 1..5: note = Description of sequence: Class II HLA motif
QQRAA

SEQ ID NO: 114 - protein, 5 aa, Homo sapiens
  REGION 1..5: note = Description of sequence: Class II HLA motif
KKRAA

SEQ ID NO: 115 - protein, 5 aa, Homo sapiens
  REGION 1..5: note = Description of sequence: Class II HLA motif
RRRAA

SEQ ID NO: 116 - protein, 5 aa, Homo sapiens
  REGION 1..5: note = Description of sequence: Class II HLA motif
QRRAA

SEQ ID NO: 117 - protein, 5 aa, Homo sapiens
  REGION 1..5: note = Description of sequence: Class II HLA motif
RKRAA

SEQ ID NO: 118 - protein, 112 aa, Homo sapiens
  REGION 1..112: note = Description of sequence: TGF-Beta-2 sequence with C77S
ALDAAYCFRNVQDNCCLRPLYIDFKRDLGWKWIHEPKGYNANFCAGACPYLWSSDTQHSRVLSLYNTINPEASASPSCVSQDLEPLTILYYIGKTPKIEQLSNMIVKSCKCS

SEQ ID NO: 119 - protein, 426 aa, synthetic construct
  REGION 1..426: note = Description of sequence: MHC-class II protein construct
EDIVADHVASYGVNLYQSYGPSGQYTHEFDGDEQFYVDLGRKETVWCLPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTEGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNKTTPPVLDSDGSFFLYSKLTVDKSRWQQGNVFSCSVMHEALHNHYTQKSLSLSPG

SEQ ID NO: 120 - protein, 225 aa, synthetic construct
  REGION 1..225: note = Description of sequence: MHC-class II protein construct
APWIEQEAPEYWGGGGSGGGGSGGGGSRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSK

SEQ ID NO: 121 - protein, 427 aa, synthetic construct
  REGION 1..427: note = Description of sequence: MHC-class II protein construct
EDIVADHVASYGVNLYQSYGPSGQYTHEFDGDEQFYVDLGRKETVWCLPVLRQFRFDPQFALTNIAVLKHNLNILIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTEGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 122 - protein, 674 aa, synthetic construct
  REGION 1..674: note = Description of sequence: MHC-class II protein construct
ADAAPWIEQEAPEYWGGGSGGGSGGGSRDSPDEFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKGGGSGGGSGGGSGGGSGGGSEDIVADHVASYGVNLYQSYGPSGQYTHEFDGDEQFYVDLGRKETVWSLPVLHQFRFDPQFALTNIAVLKHNLNILIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTEGGSAAAGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 123 - protein, 680 aa, synthetic construct
  REGION 1..680: note = Description of sequence: MHC-class II protein construct
ADAAPWIEQEAPEYWGGGGSGGGGSGGGGSRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKGGGGSGGGGSGGGGSGGGGSGGGGSEDIVADHVASYGVNLYQSYGPSGQYTHEFDGDEQFYVDLGRKETVWKLPVLHQFRFDPQFALTNIAVLKHNLNILIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTEGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 124 - protein, 680 aa, synthetic construct
  REGION 1..680: note = Description of sequence: MHC-class II protein construct
  DISULFID 35..336: note = Cysteine bond
ADAAPWIEQEAPEYWGGGGSGGGGSGGGGSRDSPCDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKGGGGSGGGGSGGGGSGGGGSGGGGSEDIVADHVASYGVNLYQSYGPSGQYTHEFDGDEQFYVDLGRKETVWCLPVLHQFRFDPQFALTNIAVLKHNLNILIKRSNSTCATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTEGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 125 - protein, 680 aa, synthetic construct
  REGION 1..680: note = Description of sequence: MHC-class II protein construct
  DISULFID 45..336: note = Cysteine bond
ADAAPWIEQEAPEYWGGGGSGGGGSGGGGSRDSPCDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKGGGGSGGGGSGGGGSGGGGSGGGGSEDIVADHVASYGVNLYQSYGPSGQYTHEFDGDEQFYVDLGRKETVWSLPVLHQFRFDPQFALTNIAVLKHNLNILIKRSNSTCATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTEGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 126 - protein, 680 aa, synthetic construct
  REGION 1..680: note = Description of sequence: MHC-class II protein construct
  DISULFID 35..336: note = Cysteine bond
ADAAPWIEQEAPEYWGGGGSGGGGSGGGGSRDSPCDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKGGGGSGGGGSGGGGSGGGGSGGGGSEDIVADHVASYGVNLYQSYGPSGQYTHEFDGDEQFYVDLGRKETVWKLPVLHQFRFDPQFALTNIAVLKHNLNILIKRSNSTCATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTEGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 127 - protein, 427 aa, synthetic construct
  REGION 1..427: note = Description of sequence: MHC-class II protein construct
EDIVADHVASYGVNLYQSYGPSGQYTHEFDGDEQFYVDLGRKETVWKLPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTEGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 128 - protein, 427 aa, synthetic construct
  REGION 1..427: note = Description of sequence: MHC-class II protein construct
EDIVADHVASYGVNLYQSYGPSGQYTHEFDGDEQFYVDLGRKETVWCLPVLHQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTEGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 129 - protein, 427 aa, synthetic construct
  REGION 1..427: note = Description of sequence: MHC-class II protein construct
EDIVADHVASYGVNLYQSYGPSGQYTHEFDGDEQFYVDLGRKETVWCLPVLRQFRFDPQFALTNIAVLKHNLNSLIKRSNSTCATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTEGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 130 - protein, 225 aa, synthetic construct
  REGION 1..225: note = Description of sequence: MHC-class II protein construct
APWIEQEAPEYWGGGGSGGGGSGGGGSRDSPCDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSK

SEQ ID NO: 131 - protein, 677 aa, synthetic construct
  REGION 1..677: note = Description of sequence: MHC-class II protein construct
APWIEQEAPEYWGGGGSGGGGSGGGGSRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKGGGGSGGGGSGGGGSGGGGSGGGGSEDIVADHVASYGVNLYQSYGPSGQYTHEFDGDEQFYVDLGRKETVWCLPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTEGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 132 - protein, 677 aa, synthetic construct
  REGION 1..677: note = Description of sequence: MHC-class II protein construct
  DISULFID 32..333: note = Cysteine bond
APWIEQEAPEYWGGGGSGGGGSGGGGSRDSPCDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKGGGGSGGGGSGGGGSGGGGSGGGGSEDIVADHVASYGVNLYQSYGPSGQYTHEFDGDEQFYVDLGRKETVWCLPVLRQFRFDPQFALTNIAVLKHNLNSLIKRSNSTCATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTEGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 133 - protein, 677 aa, synthetic construct
  REGION 1..677: note = Description of sequence: MHC-class II protein construct
APWIEQEAPEYWGGGGSGGGGSGGGGSRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKGGGGSGGGGSGGGGSGGGGSGGGGSEDIVADHVASYGVNLYQSYGPSGQYTHEFDGDEQFYVDLGRKETVWKLPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTEGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 134 - protein, 677 aa, synthetic construct
  REGION 1..677: note = Description of sequence: MHC-class II protein construct
  DISULFID 32..333: note = Cysteine bond
APWIEQEAPEYWGGGGSGGGGSGGGGSRDSPCDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKGGGGSGGGGSGGGGSGGGGSGGGGSEDIVADHVASYGVNLYQSYGPSGQYTHEFDGDEQFYVDLGRKETVWKLPVLRQFRFDPQFALTNIAVLKHNLNSLIKRSNSTCATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTEGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 135 - protein, 677 aa, synthetic construct
  REGION 1..677: note = Description of sequence: MHC-class II protein construct
  DISULFID 32..333: note = Cysteine bond
APWIEQEAPEYWGGGGSGGGGSGGGGSRDSPCDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKGGGGSGGGGSGGGGSGGGGSGGGGSEDIVADHVASYGVNLYQSYGPSGQYTHEFDGDEQFYVDLGRKETVWKLPVLHQFRFDPQFALTNIAVLKHNLNSLIKRSNSTCATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTEGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 136 - protein, 677 aa, synthetic construct
  REGION 1..677: note = Description of sequence: MHC-class II protein construct
  DISULFID 32..333: note = Cysteine bond
APWIEQEAPEYWGGGGSGGGGSGGGGSRDSPCDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKGGGGSGGGGSGGGGSGGGGSGGGGSEDIVADHVASYGVNLYQSYGPSGQYTHEFDGDEQFYVDLGRKETVWKLPVLHQFRFDPQFALTNIAVLKHNLNILIKRSNSTCATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTEGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 137 - protein, 677 aa, synthetic construct
  REGION 1..677: note = Description of sequence: MHC-class II protein construct
  DISULFID 32..333: note = Cysteine bond
APWIEQEAPEYWGGGGSGGGGSGGGGSRDSPCDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKGGGGSGGGGSGGGGSGGGGSGGGGSEDIVADHVASYGVNLYQSYGPSGQYTHEFDGDEQFYVDLGRKETVWSLPVLRQFRFDPQFALTNIAVLKHNLNSLIKRSNSTCATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTEGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 138 - protein, 657 aa, synthetic construct
  REGION 1..657: note = Description of sequence: MHC-class II protein construct
  DISULFID 33..323: note = Cysteine bond
EARPALLTSRLRFIPKGGGGSGGGGSGGGGSPCNYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEWKAQGGGGSGGGGSGGGGSGGGGSGGGGSIKADHVSTYAAFVQTHRPTGEFMFEFDEDEMFYVDLDKKETVWHLEEFGQAFSFEAQGGLANIAILNNNLNTLIQRSNHTCATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 139 - protein, 652 aa, synthetic construct
  REGION 1..652: note = Description of sequence: MHC-class II protein construct
  DISULFID 33..318: note = Cysteine bond
QHFVQENYLEYGGGGSGGGGSGGGGSPCNYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEWKAQGGGGSGGGGSGGGGSGGGGSGGGGSIKADHVSTYAAFVQTHRPTGEFMFEFDEDEMFYVDLDKKETVWHLEEFGQAFSFEAQGGLANIAILNNNLNTLIQRSNHTCATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 140 - protein, 914 aa, synthetic construct
  REGION 1..914: note = Description of sequence: MHC-class II protein construct
  DISULFID 32..332: note = Cysteine bond
  DOMAIN 233..245: note = TIMGRASS
  DOMAIN 1..13: note = linker
  DOMAIN 14..28: note = h DRB*0401 beta domains (P5C)
  DOMAIN 29..227: note = membrane proximal region
  DOMAIN 228..252: note = linker
  DOMAIN 253..441: note = HLA-DRA*0101 alpha membrane proximal region
  DOMAIN 442..449: note = linker
  DOMAIN 450..675: note = hIgG1 ("LALA")
  DOMAIN 676..695: note = linker
  DOMAIN 696..914: note = hPDL
SYKFIPALEAAVGGGGSGGGGSGGGGSGDTRCRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKMGGGGSGGGGSGGGGSGGGGSGGGGSIKEEHVIIQAEFYLNPDQSGEFMFDFDGDEIFHVDMAKKETVWRLEEFGRFASFEAQGALANIAVDKANLEIMTKRSNYTCITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 141 - protein, 956 aa, synthetic construct
  REGION 1..956: note = Description of sequence: MHC-class II protein construct
  DISULFID 31..321: note = Cysteine bond
LLTQHFVQENYLEYGGGGSGGGGSGGGGSPCNYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEWKAQGGGGSGGGGSGGGGSGGGGSGGGGSIKADHVSTYAAFVQTHRPTGEFMFEFDEDEMFYVDLDKKETVWHLEEFGQAFSFEAQGGLANIAILNNNLNTLIQRSNHTCATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 142 - protein, 675 aa, synthetic construct
  REGION 1..675: note = Description of sequence: MHC-class II protein construct
  DISULFID 33..333: note = Cysteine bond
ESYKFIPALEAAVGGGGSGGGGSGGGGSGDTRCRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKMGGGGSGGGGSGGGGSGGGGSGGGGSIKEEHVIIQAEFYLNPDQSGEFMFDFDGDEIFHVDMAKKETVWRLEEFGRFASFEAQGALANIAVDKANLEIMTKRSNYTCITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 143 - protein, 958 aa, synthetic construct
  REGION 1..958: note = Description of sequence: MHC-class II protein construct
  DISULFID 33..323: note = Cysteine bond
EARPALLTSRLRFIPKGGGGSGGGGSGGGGSPCNYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEWKAQGGGGSGGGGSGGGGSGGGGSGGGGSIKADHVSTYAAFVQTHRPTGEFMFEFDEDEMFYVDLDKKETVWHLEEFGQAFSFEAQGGLANIAILNNNLNTLIQRSNHTCATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 144 - protein, 679 aa, synthetic construct
  REGION 1..679: note = Description of sequence: MHC-class II protein construct
  DISULFID 37..337: note = Cysteine bond
ENPVVHFFKNIVTPRTPGGGGSGGGGSGGGGSGDTRCRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSKMGGGGSGGGGSGGGGSGGGGSGGGGSIKEEHVIIQAEFYLNPDQSGEFMFDFDGDEIFHVDMAKKETVWRLEEFGRFASFEAQGALANIAVDKANLEIMTKRSNYTCITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 145 - protein, 828 aa, synthetic construct
  REGION 1..828: note = Description of sequence: MHC-class II protein construct
  DISULFID 33..333: note = Cysteine bond
  DOMAIN 233..245: note = TIMGRASS
  DOMAIN 1..13: note = linker
  DOMAIN 14..28: note = h DRB*0401 beta domains (P5C)
  DOMAIN 29..227: note = membrane proximal region
  DOMAIN 228..252: note = linker
  DOMAIN 253..441: note = HLA-DRA*0101 alpha membrane proximal region
  DOMAIN 442..449: note = linker
  DOMAIN 450..675: note = hIgG1 ("LALA")
  DOMAIN 676..695: note = linker
  DOMAIN 696..828: note = 2 (H16A, F42A
ESYKFIPALEAAVGGGGSGGGGSGGGGSGDTRCRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKMGGGGSGGGGSGGGGSGGGGSGGGGSIKEEHVIIQAEFYLNPDQSGEFMFDFDGDEIFHVDMAKKETVWRLEEFGRFASFEAQGALANIAVDKANLEIMTKRSNYTCITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 146 - protein, 812 aa, synthetic construct
  REGION 1..812: note = Description of sequence: MHC-class II protein construct
  DISULFID 33..333: note = Cysteine bond
  DOMAIN 233..245: note = TIMGRASS
  DOMAIN 1..13: note = linker
  DOMAIN 14..28: note = h DRB*0401 beta domains (P5C)
  DOMAIN 29..227: note = membrane proximal region
  DOMAIN 228..252: note = linker
  DOMAIN 253..441: note = HLA-DRA*0101 alpha membrane proximal region
  DOMAIN 442..449: note = linker
  DOMAIN 450..675: note = hIgG1(CH3 S)..(S KIH knob S354C, T366W, and "LALA")
  DOMAIN 676..700: note = hTGFbeta
  DOMAIN 701..812: note = hTGF-beta-3 (C77S)
ESYKFIPALEAAVGGGGSGGGGSGGGGSGDTRCRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKMGGGGSGGGGSGGGGSGGGGSGGGGSIKEEHVIIQAEFYLNPDQSGEFMFDFDGDEIFHVDMAKKETVWRLEEFGRFASFEAQGALANIAVDKANLEIMTKRSNYTCITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSALDTNYCFRNLEENCCVRPLYIDFRQDLGWKWVHEPKGYYANFCSGPCPYLRSADTTHSTVLGLYNTLNPEASASPSCVPQDLEPLTILYYVGRTPKVEQLSNMVVKSCKCS

SEQ ID NO: 147 - protein, 801 aa, synthetic construct
  REGION 1..801: note = Description of sequence: MHC-class II protein construct
  DISULFID 33..333: note = Cysteine bond
  DOMAIN 233..245: note = TIMGRASS
  DOMAIN 1..13: note = linker
  DOMAIN 14..28: note = h DRB*0401 beta domains (P5C)
  DOMAIN 29..227: note = membrane proximal region
  DOMAIN 228..252: note = linker
  DOMAIN 253..441: note = HLA-DRA*0101 alpha membrane proximal region
  DOMAIN 442..449: note = linker
  DOMAIN 450..675: note = hIgG1(KIH Y349C, T366S, L368A, Y407V, and "LALA")
  DOMAIN 676..690: note = linker
  DOMAIN 691..801: note = hTBRII (delta 25 D118A)
ESYKFIPALEAAVGGGGSGGGGSGGGGSGDTRCRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKMGGGGSGGGGSGGGGSGGGGSGGGGSIKEEHVIIQAEFYLNPDQSGEFMFDFDGDEIFHVDMAKKETVWRLEEFGRFASFEAQGALANIAVDKANLEIMTKRSNYTCITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSQLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEEYNTSNPD

SEQ ID NO: 148 - protein, 954 aa, synthetic construct
  REGION 1..954: note = Description of sequence: MHC-class II protein construct
  DISULFID 33..333: note = Cysteine bond
  DOMAIN 233..245: note = TIMGRASS
  DOMAIN 1..13: note = linker
  DOMAIN 14..28: note = h DRB*0401 beta domains (P5C)
  DOMAIN 29..227: note = membrane proximal region
  DOMAIN 228..252: note = linker
  DOMAIN 253..441: note = HLA-DRA*0101 alpha membrane proximal region
  DOMAIN 442..449: note = linker
  DOMAIN 450..675: note = hIgG1(KIH Y349C, T366S, L368A, Y407V, and "LALA")
  DOMAIN 676..690: note = linker
  DOMAIN 691..801: note = hTBRII (delta 25, D118A)
  DOMAIN 822..954: note = 2 (H16A, F42A)
ESYKFIPALEAAVGGGGSGGGGSGGGGSGDTRCRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKMGGGGSGGGGSGGGGSGGGGSGGGGSIKEEHVIIQAEFYLNPDQSGEFMFDFDGDEIFHVDMAKKETVWRLEEFGRFASFEAQGALANIAVDKANLEIMTKRSNYTCITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSQLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEEYNTSNPDGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 149 - protein, 855 aa, synthetic construct
  REGION 1..855: note = Description of sequence: MHC-class II protein construct
  DISULFID 33..333: note = Cysteine bond
  DOMAIN 233..245: note = TIMGRASS
  DOMAIN 1..13: note = linker
  DOMAIN 14..28: note = h DRB*0401 beta domains (P5C)
  DOMAIN 29..227: note = membrane proximal region
  DOMAIN 228..252: note = linker
  DOMAIN 253..441: note = HLA-DRA*0101 alpha membrane proximal region
  DOMAIN 442..449: note = linker
  DOMAIN 450..675: note = hIgG1(KIH Y349C, T366S, L368A, Y407V and "LALA" )
  DOMAIN 676..690: note = linker
  DOMAIN 691..855: note = hIL-10
ESYKFIPALEAAVGGGGSGGGGSGGGGSGDTRCRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKMGGGGSGGGGSGGGGSGGGGSGGGGSIKEEHVIIQAEFYLNPDQSGEFMFDFDGDEIFHVDMAKKETVWRLEEFGRFASFEAQGALANIAVDKANLEIMTKRSNYTCITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 150 - protein, 124 aa, synthetic construct
  REGION 1..124: note = Description of sequence: Control: MHC-Class I Protein construct
YMLDLQPETTGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 151 - protein, 507 aa, synthetic construct
  REGION 1..507: note = Description of sequence: Control: MHC-Class I Protein construct
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 229 aa, synthetic construct
  REGION 1..229: note = Description of sequence: MHC-class II protein construct
SLQPLALEGSLQSRGGGGGSGGGGSGGGGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 153 - protein, 423 aa, synthetic construct
  REGION 1..423: note = Description of sequence: MHC-class II protein construct
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 154 - protein, 665 aa, synthetic construct
  REGION 1..665: note = Description of sequence: MHC-class II protein construct
GGGGSGGGGSGGGGSRDSPCDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKGGGGSGGGGSGGGGSGGGGSGGGGSEDIVADHVASYGVNLYQSYGPSGQYTHEFDGDEQFYVDLGRKETVWKLPVLHQFRFDPQFALTNIAVLKHNLNILIKRSNSTCATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTEGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 155 - protein, 662 aa, synthetic construct
  REGION 1..662: note = Description of sequence: MHC-class II protein construct
  DISULFID 20..320: note = Cysteine bond
GGGGSGGGGSGGGGSGDTRCRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKMGGGGSGGGGSGGGGSGGGGSGGGGSIKEEHVIIQAEFYLNPDQSGEFMFDFDGDEIFHVDMAKKETVWRLEEFGRFASFEAQGALANIAVDKANLEIMTKRSNYTCITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 156 - protein, 662 aa, synthetic construct; identical to SEQ ID NO: 155
  REGION 1..662: note = Description of sequence: MHC-class II protein construct

SEQ ID NO: 157 - protein, 390 aa, Homo sapiens
  REGION 1..390: note = Description of sequence: TGF-Beta1 preproprotein
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 158 - protein, 390 aa, Homo sapiens
  REGION 1..390: note = Description of sequence: UniProtKB - P01137 having a P10L
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 159 - protein, 413 aa, Homo sapiens
  REGION 1..413: note = Description of sequence: Homo sapiens TGF-Beta2 preproprotein
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSEAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 160 - protein, 412 aa, Homo sapiens
  REGION 1..412: note = Description of sequence: Homo sapiens TGF-Beta3 preproprotein isoform 1
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 161 - protein, 112 aa, Homo sapiens [sequence omitted]

SEQ ID NO: 162 - protein, 112 aa, Homo sapiens
  REGION 1..112: note = Description of sequence: Homo sapiens Mature TGF-Beta3 isoform 1 Mutant: C77S
ALDTNYCFRNLEENCCVRPLYIDFRQDLGWKWVHEPKGYYANFCSGPCPYLRSADTTHSTVLGLYNTLNPEASASPSCVPQDLEPLTILYYVGRTPKVEQLSNMVVKSCKCS

SEQ ID NO: 163 - protein, 503 aa, Homo sapiens
  REGION 1..503: note = Description of sequence: Homo sapiens TGF-Beta receptor type-1 isoform 1 precursor
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 164 - protein, 93 aa, Homo sapiens
  REGION 1..93: note = Description of sequence: Homo sapiens TGF-Beta receptor type-1 isoform 1 ectodomain
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVEL

SEQ ID NO: 165 - protein, 592 aa, Homo sapiens
  REGION 1..592: note = Description of sequence: Homo sapiens TGF-Beta receptor type-2 (T-Beta-RII) isoform A precursor
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 166 - protein, 154 aa, Homo sapiens
  REGION 1..154: note = Description of sequence: Homo sapiens TGF-Beta receptor type-2 (T-Beta-RII) isoform A ectodomain
IPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEE

SEQ ID NO: 167 - protein, 567 aa, Homo sapiens
  REGION 1..567: note = Description of sequence: Homo sapiens TGF-Beta receptor type-2 (T-Beta-RII) isoform B precursor
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 168 - protein, 143 aa, Homo sapiens
  REGION 1..143: note = Description of sequence: Homo sapiens TGF-Beta receptor type-2 (T-Beta-RII) isoform B ectodomain sequence
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 169 - protein, 129 aa, Homo sapiens
  REGION 1..129: note = Description of sequence: Homo sapiens TGF-Beta receptor type-2 (T-Beta-RII) isoform B ectodomain fragment sequence
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEE

SEQ ID NO: 170 - protein, 115 aa, Homo sapiens
  REGION 1..115: note = Description of sequence: Homo sapiens TGF-Beta receptor type-2 (T-Beta-RII) isoform B ectodomain fragment Delta14 (N-terminus) D118A
VTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEE

SEQ ID NO: 171 - protein, 104 aa, Homo sapiens
  REGION 1..104: note = Description of sequence: Homo sapiens TGF-Beta receptor type-2 (T-Beta-RII) isoform B ectodomain fragment Delta25 (N-terminus) D118A
QLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEE

SEQ ID NO: 172 - protein, 111 aa, Homo sapiens
  REGION 1..111: note = Description of sequence: Homo sapiens TGF-Beta receptor type-2 (T-Beta-RII) isoform B Delta 25-Nterm D118A ectodomain
QLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEEYNTSNPD

SEQ ID NO: 173 - protein, 851 aa, Homo sapiens
  REGION 1..851: note = Description of sequence: Homo sapiens TGF-Beta receptor type III (3) isoform A precursor (BETA Glycan)
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 174 - protein, 850 aa, Homo sapiens
  REGION 1..850: note = Description of sequence: Homo sapiens TGF-Beta receptor type III (3) isoform B precursor (BETA Glycan)
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 175 - protein, 12 aa, Homo sapiens
  REGION 1..12: note = Description of sequence: gamma-gliadin
QLQPFPQPELPY

SEQ ID NO: 176 - protein, 14 aa, Homo sapiens
  REGION 1..14: note = Description of sequence: gamma-gliadin
QPFPQPELPYPQPE

SEQ ID NO: 177 - protein, 11 aa, Homo sapiens
  REGION 1..11: note = Description of sequence: gamma-gliadin
PQPELPYPQPE

SEQ ID NO: 178 - protein, 15 aa, Homo sapiens
  REGION 1..15: note = Description of sequence: InsA:1-15
GIVDQCCTSICSLYQ

SEQ ID NO: 179 - protein, 13 aa, Homo sapiens
  REGION 1..13: note = Description of sequence: GAD65:555-567
NFFRMVISNPAAT

SEQ ID NO: 180 - protein, 20 aa, Homo sapiens
  REGION 1..20: note = Description of sequence: IA2:853-872
SFYLKNVQTQETRTLTQFHF

SEQ ID NO: 181 - protein, 15 aa, Homo sapiens
  REGION 1..15: note = Description of sequence: InsB:9-23;
SHLVEALYLVCGERG

SEQ ID NO: 182 - protein, 20 aa, Homo sapiens
  REGION 1..20: note = Description of sequence: GAD65 peptides 121-140
YVVKSFDRSTKVIDFHYPNE

SEQ ID NO: 183 - protein, 17 aa, Homo sapiens
  REGION 1..17: note = Description of sequence: GAD65 peptides 250-266
AMMIARFKMFPEVKEKG

SEQ ID NO: 184 - protein, 9 aa, Homo sapiens
  REGION 1..9: note = Description of sequence: Pro-insulin C-peptide:66-74;
VELGGGPGA

SEQ ID NO: 185 - protein, 14 aa, Homo sapiens
  REGION 1..14: note = Description of sequence: IGRP
VYLKTNVFLGGGAS

SEQ ID NO: 186 - protein, 12 aa, Homo sapiens
  REGION 1..12: note = Description of sequence: a1 gliadin
QLQIFPEPEEPY

SEQ ID NO: 187 - protein, 12 aa, Homo sapiens
  REGION 1..12: note = Description of sequence: a2 gliadin
QPQPELPYPQPE

SEQ ID NO: 188 - protein, 11 aa, Homo sapiens
  REGION 1..11: note = Description of sequence: a2 gliadin
IQPELPEPQPE

SEQ ID NO: 189 - protein, 83 aa, Homo sapiens
  REGION 1..83: note = Description of sequence: DRA alpha 1
IKEEVIIQAEFYLNPDQSGEFMFDFDGDEIFHVDMAKKETVWRLEEFGRFASFEAQGALANIAVDKANLEIMTKRSNYTPITN

SEQ ID NO: 190 - protein, 94 aa, Homo sapiens
  REGION 1..94: note = Description of sequence: DRA alpha 2
VPPEVTVLTNSPVELREPNVLICFIDKFTPPVVNVTWLRNGKPVTTGVSETVFLPREDHLFRKFHYLPFLPSTEDVYDCRVEHWGLDEPLLKHW

SEQ ID NO: 191 - protein, 87 aa, Homo sapiens
  REGION 1..87: note = Description of sequence: DPA a1
AGAIKADHVSTYAAFVQTHRPTGEFMFEFDEDEMFYVDLDKKETVWHLEEFGQAFSFEAQGGLANIAILNNNLNTLIQRSNHTQATN

SEQ ID NO: 192 - protein, 94 aa, Homo sapiens
  REGION 1..94: note = Description of sequence: DPA a2
DPPEVTVFPKEPVELGQPNTLICHIDKFFPPVLNVTWLCNGELVTEGVAESLFLPRTDYSFHKFHYLTFVPSAEDFYDCRVEHWGLDQPLLKHW

SEQ ID NO: 193 - protein, 181 aa, Homo sapiens
  REGION 1..181: note = Description of sequence: DQA2*01:01 a1 and a2 domain sequences:
EDIVADHVASYGVNFYQSHGPSGQYTHEFDGDEEFYVDLETKETVWQLPMFSKFISFDPQSALRNMAVGKHTLEFMMRQSNSTAATNEVPEVTVFSKFPVTLGQPNTLICLVDNIFPPVVNITWLSNGHSVTEGVSETSFLSKSDHSFFKISYLTFLPSADEIYDCKVEHWGLDEPLLKHW

SEQ ID NO: 194 - protein, 4 aa, Homo sapiens
  REGION 1..4: note = Description of sequence: Subsequence
PRFL

SEQ ID NO: 195 - protein, 5 aa, Homo sapiens
  REGION 1..5: note = Description of sequence: Subsequence
PENYL

SEQ ID NO: 196 - protein, 5 aa, Homo sapiens
  REGION 1..5: note = Description of sequence: Subsequence
PENYV

SEQ ID NO: 197 - protein, 4 aa, Homo sapiens
  REGION 1..4: note = Description of sequence: Subsequence
PEDF

SEQ ID NO: 198 - protein, 5 aa, Homo sapiens
  REGION 1..5: note = Description of sequence: Subsequence
PKDFL

SEQ ID NO: 199 - protein, 4 aa, Homo sapiens
  REGION 1..4: note = Description of sequence: Subsequence
PENY

SEQ ID NO: 200 - protein, 33 aa, Homo sapiens
  REGION 1..33: note = Description of sequence: a2 gliadin 33mer
LQLQPFPQPELPYPQPELPYPQPELPYPQPQPF

SEQ ID NO: 201 - protein, 19 aa, Homo sapiens
  REGION 1..19: note = Description of sequence: a2 gliadin 19mer
LGQQQPFPPQQPYPQPQPF

SEQ ID NO: 202 - protein, 112 aa, Homo sapiens
  REGION 1..112: note = Description of sequence: TGF-Beta1
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 203 - protein, 112 aa, Homo sapiens
  REGION 1..112: note = Description of sequence: TGF-Beta1 C77S
ALDTNYCFSSTEKNCCVRQLYIDFRKDLGWKWIHEPKGYHANFCLGPCPYIWSLDTQYSKVLALYNQHNPGASAAPSCVPQALEPLPIVYYVGRKPKVEQLSNMIVRSCKCS

SEQ ID NO: 204 - protein, 112 aa, Homo sapiens; identical to SEQ ID NO: 118
  REGION 1..112: note = Description of sequence: TGF-Beta-3 sequence

SEQ ID NO: 205 - protein, 115 aa, Homo sapiens
  REGION 1..115: note = Description of sequence: T-beta-RII isoform B ectodomain delta 1-14
VTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEE

SEQ ID NO: 206 - protein, 104 aa, Homo sapiens
  REGION 1..104: note = Description of sequence: T-beta-RII isoform B ectodomain delta 1-14
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEE

SEQ ID NO: 207 - protein, 248 aa, Homo sapiens
  REGION 1..248: note = Description of sequence: cis-masked TGF-Beta MOD
QLCKFCDVRFSTCDNQKSCMSNCSITSICEKPQEVCVAVWRKNDENITLETVCHDPKLPYHDFILEDAASPKCIMKEKKKPGETFFMCSCSSAECNDNIIFSEEYNTSNPDGGGGSGGGGSGGGGSGGGGSGGGGSALDTNYCFRNLEENCCVRPLYIDFRQDLGWKWVHEPKGYYANFCSGPCPYLRSADTTHSTVLGLYNTLNPEASASPSCVPQDLEPLTILYYVGRTPKVEQLSNMVVKSCKCS

SEQ ID NO: 208 - protein, 133 aa, Homo sapiens [sequence omitted]

SEQ ID NO: 209 - protein, 133 aa, Homo sapiens
  REGION 1..133: note = Description of sequence: variant IL-2 polypeptide
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 210 - protein, 245 aa, Homo sapiens
  REGION 1..245: note = Description of sequence: human PD-L1 polypeptide
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKICLTLSPST

SEQ ID NO: 211 - protein, 219 aa, Homo sapiens
  REGION 1..219: note = Description of sequence: wild-type human PD-L1 ectodomain
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 212 - protein, 227 aa, Homo sapiens
  REGION 1..227: note = Description of sequence: wild-type human PD-L1 ectodomain
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERLNVSIKI

SEQ ID NO: 213 - protein, 124 aa, Homo sapiens
  REGION 1..124: note = Description of sequence: wild-type PD-L1 IgV domain
AFTVTVPKDLYVVEYGSNMTIECKFPVEKQLDLAALIVYWEMEDKNIIQFVHGEEDLKTQHSSYRQRARLLKDQLSLGNAALQITDVKLQDAGVYRCMISYGGADYKRITVKVNAPYAAALHEH

SEQ ID NO: 214 - protein, 268 aa, Homo sapiens
  REGION 1..268: note = Description of sequence: wild-type PD-1
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 215 - protein, 160 aa, Homo sapiens
  REGION 1..160: note = Description of sequence: wild-type IL-10
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 216 - protein, 9 aa, Homo sapiens
  REGION 1..9: note = Description of sequence: nonapeptide
SPGQGTQSE

SEQ ID NO: 217 - protein, 9 aa, Homo sapiens
  REGION 1..9: note = Description of sequence: nonapeptide
AYMTMKIRN

SEQ ID NO: 218 - protein, 160 aa, Homo sapiens
  REGION 1..160: note = Description of sequence: IL-10 D25A and/or E97A
SPGQGTQSENSCTHFPGNLPNMLRXLRDAFSRVKTFFQMKDQLDNLLLKESLLEDFKGYLGCQALSEMIQFYLEEVMPQAENQDPDIKAHVNSLGXNLKTLRLRLRRCHRFLPCENKSKAVEQVKNAFNKLQEKGIYKAMSEFDIFINYIEAYMTMKIRN

SEQ ID NO: 219 - protein, 160 aa, Homo sapiens
  REGION 1..160: note = Description of sequence: IL-10 D25A E97A
SPGQGTQSENSCTHFPGNLPNMLRALRDAFSRVKTFFQMKDQLDNLLLKESLLEDFKGYLGCQALSEMIQFYLEEVMPQAENQDPDIKAHVNSLGANLKTLRLRLRRCHRFLPCENKSKAVEQVKNAFNKLQEKGIYKAMSEFDIFINYIEAYMTMKIRN

SEQ ID NO: 220 - protein, 33 aa, synthetic construct
  REGION 1..33: note = Description of sequence: Leucine Zipper
RMKQIEDKIEEILSKIYHIENEIARIKKLIGER

SEQ ID NO: 221 - protein, 32 aa, synthetic construct
  REGION 1..32: note = Description of sequence: Leucine Zipper
LSSIEKKQEEQTSWLIWISNELTLIRNELAQS

SEQ ID NO: 222 - protein, 31 aa, synthetic construct
  REGION 1..31: note = Description of sequence: Leucine Zipper
LSSIEKKLEEITSQLIQISNELTLIRNELAQ

SEQ ID NO: 223 - protein, 31 aa, synthetic construct
  REGION 1..31: note = Description of sequence: Leucine Zipper
LSSIEKKLEEITSQLIQIRNELTLIRNELAQ

SEQ ID NO: 224 - protein, 31 aa, synthetic construct
  REGION 1..31: note = Description of sequence: Leucine Zipper
LSSIEKKLEEITSQLQQIRNELTLIRNELAQ

SEQ ID NO: 225 - protein, 31 aa, synthetic construct
  REGION 1..31: note = Description of sequence: Leucine Zipper
LSSLEKKLEELTSQLIQLRNELTLLRNELAQ

SEQ ID NO: 226 - protein, 31 aa, synthetic construct
  REGION 1..31: note = Description of sequence: Leucine Zipper
ISSLEKKIEELTSQIQQLRNEITLLRNEIAQ

SEQ ID NO: 227 - protein, 47 aa, synthetic construct
  REGION 1..47: note = Description of sequence: Leucine Zipper
LEIEAAFLERENTALETRVAELRQRVQRLRNRVSQYRTRYGPLGGGK

SEQ ID NO: 228 - protein, 31 aa, synthetic construct
  REGION 1..31: note = Description of sequence: coiled coil
LKSVENRLAVVENQLKTVIEELKTVKDLLSN

SEQ ID NO: 229 - protein, 31 aa, synthetic construct
  REGION 1..31: note = Description of sequence: coiled coil
LARIEEKLKTIKAQLSEIASTLNMIREQLAQ

SEQ ID NO: 230 - protein, 31 aa, synthetic construct
  REGION 1..31: note = Description of sequence: coiled coil
VSRLEEKVKTLKSQVTELASTVSLLREQVAQ

SEQ ID NO: 231 - protein, 30 aa, synthetic construct
  REGION 1..30: note = Description of sequence: coiled coil
IQSEKKIEDISSLIGQIQSEITLIRNEIAQ

SEQ ID NO: 232 - protein, 31 aa, synthetic construct
  REGION 1..31: note = Description of sequence: coiled coil
LMSLEKKLEELTQTLMQLQNELSMLKNELAQ

SEQ ID NO: 233 - protein, 18 aa, synthetic construct
  REGION 1..18: note = Description of sequence: Cysteine
VDLEGSTSNGRQCAGIRL

SEQ ID NO: 234 - protein, 27 aa, synthetic construct
  REGION 1..27: note = Description of sequence: Cysteine
EDDVTTTEELAPALVPPPKGTCAGWMA

SEQ ID NO: 235 - protein, 27 aa, synthetic construct
  REGION 1..27: note = Description of sequence: Cysteine
GHDQETTTQGPGVLLPLPKGACTGQMA

SEQ ID NO: 236 - protein, 64 aa, synthetic construct
  REGION 1..64: note = Description of sequence: Collagen
VTAFSNMDDMLQKAHLVIEGTFIYLRDSTEFFIRVRDGWKKLQLGELIPIPADSPPPPALSSNP

SEQ ID NO: 237 - protein, 5 aa, synthetic construct
  REGION 1..5: note = Description of sequence: Linker
GGGGS

SEQ ID NO: 238 - protein, 4 aa, synthetic construct
  REGION 1..4: note = Description of sequence: Linker
GGGS

SEQ ID NO: 239 - protein, 4 aa, synthetic construct
  REGION 1..4: note = Description of sequence: Linker
GGSS

SEQ ID NO: 240 - protein, 5 aa, synthetic construct
  REGION 1..5: note = Description of sequence: linker
GSGGS

SEQ ID NO: 241 - protein, 4 aa, synthetic construct
  REGION 1..4: note = Description of sequence: Linker
GGSG

SEQ ID NO: 242 - protein, 5 aa, synthetic construct
  REGION 1..5: note = Description of sequence: Linker
GGSGG

SEQ ID NO: 243 - protein, 5 aa, synthetic construct
  REGION 1..5: note = Description of sequence: Linker
GSGSG

SEQ ID NO: 244 - protein, 5 aa, synthetic construct
  REGION 1..5: note = Description of sequence: Linker
GSGGG

SEQ ID NO: 245 - protein, 5 aa, synthetic construct
  REGION 1..5: note = Description of sequence: Linker
GGGSG

SEQ ID NO: 246 - protein, 5 aa, synthetic construct
  REGION 1..5: note = Description of sequence: Linker
GSSSG

SEQ ID NO: 247 - protein, 5 aa, synthetic construct
  REGION 1..5: note = Description of sequence: Linker
GSSSS

SEQ ID NO: 248 - protein, 5 aa, synthetic construct
  REGION 1..5: note = Description of sequence: Linker
AAAGG

SEQ ID NO: 249 - protein, 8 aa, synthetic construct
  REGION 1..8: note = Description of sequence: Linker
GGSAAAGG

SEQ ID NO: 250 - protein, 5 aa, synthetic construct
  REGION 1..5: note = Description of sequence: Rigid linker
EAAAK

SEQ ID NO: 251 - protein, 7 aa, synthetic construct
  REGION 1..7: note = Description of sequence: Rigid linker
AEAAAKA

SEQ ID NO: 252 - protein, 16 aa, synthetic construct
  REGION 1..16: note = Description of sequence: Rigid linker
AEAAAKALEAEAAAKA

SEQ ID NO: 253 - protein, 10 aa, synthetic construct
  REGION 1..10: note = Description of sequence: Rigid linker
EAAAKEAAAK

SEQ ID NO: 254 - protein, 15 aa, synthetic construct
  REGION 1..15: note = Description of sequence: Rigid linker
EAAAKEAAAKEAAAK

SEQ ID NO: 255 - protein, 12 aa, synthetic construct
  REGION 1..12: note = Description of sequence: Rigid linker
AEAAAKEAAAKA

SEQ ID NO: 256 - protein, 4 aa, synthetic construct
  REGION 1..4: note = Description of sequence: Rigid linker
APAP

SEQ ID NO: 257 - protein, 8 aa, synthetic construct
  REGION 1..8: note = Description of sequence: Rigid linker
APAPAPAP

SEQ ID NO: 258 - protein, 12 aa, synthetic construct
  REGION 1..12: note = Description of sequence: Rigid linker
APAPAPAPAPAP

SEQ ID NO: 259 - protein, 16 aa, synthetic construct
  REGION 1..16: note = Description of sequence: Rigid linker
APAPAPAPAPAPAPAP

SEQ ID NO: 260 - protein, 20 aa, synthetic construct
  REGION 1..20: note = Description of sequence: Rigid linker
APAPAPAPAPAPAPAPAPAP

SEQ ID NO: 261 - protein, 4 aa, synthetic construct
  REGION 1..4: note = Description of sequence: Rigid linker
KPKP

SEQ ID NO: 262 - protein, 8 aa, synthetic construct
  REGION 1..8: note = Description of sequence: Rigid linker
KPKPKPKP

SEQ ID NO: 263 - protein, 12 aa, synthetic construct
  REGION 1..12: note = Description of sequence: Rigid linker
KPKPKPKPKPKP

SEQ ID NO: 264 - protein, 16 aa, synthetic construct
  REGION 1..16: note = Description of sequence: Rigid linker
KPKPKPKPKPKPKPKP

SEQ ID NO: 265 - protein, 20 aa, synthetic construct
  REGION 1..20: note = Description of sequence: Rigid linker
KPKPKPKPKPKPKPKPKPKP

SEQ ID NO: 266 - protein, 4 aa, synthetic construct
  REGION 1..4: note = Description of sequence: Rigid linker
EPEP

SEQ ID NO: 267 - protein, 8 aa, synthetic construct
  REGION 1..8: note = Description of sequence: Rigid linker
EPEPEPEP

SEQ ID NO: 268 - protein, 12 aa, synthetic construct
  REGION 1..12: note = Description of sequence: Rigid linker
EPEPEPEPEPEP

SEQ ID NO: 269 - protein, 16 aa, synthetic construct
  REGION 1..16: note = Description of sequence: Rigid linker
EPEPEPEPEPEPEPEP

SEQ ID NO: 270 - protein, 20 aa, synthetic construct
  REGION 1..20: note = Description of sequence: Rigid linker
EPEPEPEPEPEPEPEPEPEP

SEQ ID NO: 271 - protein, 5 aa, synthetic construct
  REGION 1..5: note = Description of sequence: Disulfide linker
CGGGS

SEQ ID NO: 272 - protein, 5 aa, synthetic construct
  REGION 1..5: note = Description of sequence: Disulfide linker
GCGGS

SEQ ID NO: 273 - protein, 5 aa, synthetic construct
  REGION 1..5: note = Description of sequence: Disulfide linker
GGCGS

SEQ ID NO: 274 - protein, 5 aa, synthetic construct
  REGION 1..5: note = Description of sequence: Disulfide linker
GGGCS

SEQ ID NO: 275 - protein, 5 aa, synthetic construct
  REGION 1..5: note = Description of sequence: Disulfide linker
GGGGC

SEQ ID NO: 276 - protein, 15 aa, synthetic construct
  REGION 1..15: note = Description of sequence: L1 linker
GCGASGGGGSGGGGS

SEQ ID NO: 277 - protein, 20 aa, synthetic construct
  REGION 1..20: note = Description of sequence: L1 linker
GCGGSGGGGSGGGGSGGGGS

SEQ ID NO: 278 - protein, 15 aa, synthetic construct
  REGION 1..15: note = Description of sequence: L1 linker
GCGGSGGGGSGGGGS

SEQ ID NO: 279 - protein, 229 aa, Homo sapiens
  REGION 1..229: note = Description of sequence: insulin (InsA (1-15)
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGVRKSNAAERRGPL

SEQ ID NO: 280 - protein, 13 aa, Homo sapiens
  REGION 1..13: note = Description of sequence: GAD65 (555-567 F557I)
NFIRMVISNPAAT

SEQ ID NO: 281 - protein, 15 aa, Homo sapiens
  REGION 1..15: note = Description of sequence: proinsulin peptide
SLQPLALEGSLQSRG

SEQ ID NO: 282 - protein, 18 aa, Homo sapiens
  REGION 1..18: note = Description of sequence: proIns 75-92(K88S)
GSLQPLALEGSLQSRGIV

SEQ ID NO: 283 - protein, 110 aa, Homo sapiens
  REGION 1..110: note = Description of sequence: preproinsulin
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 284 - protein, 18 aa, Homo sapiens
  REGION 1..18: note = Description of sequence: T1D-epitope
GAGSLQPLALEGSLQKRG

SEQ ID NO: 285 - protein, 15 aa, Homo sapiens
  REGION 1..15: note = Description of sequence: T1D-epitope
SLQPLALEGSLQKRG

SEQ ID NO: 286 - protein, 13 aa, Homo sapiens
  REGION 1..13: note = Description of sequence: T1D-epitope
QPLALEGSLQKRG

SEQ ID NO: 287 - protein, 13 aa, Homo sapiens
  REGION 1..13: note = Description of sequence: T1D-epitope
QPLALEGSLQSRG

SEQ ID NO: 288 - protein, 314 aa, Homo sapiens
  REGION 1..314: note = Description of sequence: gamma-gliadin
MKTLLILTILAMATTIATANMQVDPSGQVQWPQQQPFPQPQQPFCEQPQRTIPQPHQTFHHQPQQTFPQPEQTYPHQPQQQFPQTQQPQQPFPQPQQTFPQQPQLPFPQQPQQPFPQPQQPQQPFPQSQQPQQPFPQPQQQFPQPQQPQQSFPQQQQPLIQPYLQQQMNPCKNYLLQQCNPVSLVSSLVSMILPRSDCKVMRQQCCQQLAQIPQQLQCAAIHGIVHSIIMQQEQQQQQQQQQQQQQQQGIQIMRPLFQLVQGQGIIQPQQPAQLEVIRSLVLGTLPTMCNVFVPPECSTTKAPFASIVADIGGQ

SEQ ID NO: 289 - protein, 7 aa, Homo sapiens
  REGION 1..7: note = Description of sequence: gamma-gliadin
QPFPQPQ

SEQ ID NO: 290 - protein, 9 aa, Homo sapiens
  REGION 1..9: note = Description of sequence: gamma-gliadin
PFPQPQLPY

SEQ ID NO: 291 - protein, 9 aa, Homo sapiens
  REGION 1..9: note = Description of sequence: gamma-gliadin
PFPQPELPY

SEQ ID NO: 292 - protein, 11 aa, Homo sapiens
  REGION 1..11: note = Description of sequence: gamma-gliadin
LQPFPQPELPY

SEQ ID NO: 293 - protein, 15 aa, Homo sapiens
  REGION 1..15: note = Description of sequence: gamma-gliadin
ADAQLQPFPQPELPY

SEQ ID NO: 294 - protein, 14 aa, Homo sapiens
  REGION 1..14: note = Description of sequence: gamma-gliadin
ADALQPFPQPELPY

SEQ ID NO: 295 - protein, 13 aa, Homo sapiens
  REGION 1..13: note = Description of sequence: gamma-gliadin
ADAQPFPQPELPY

SEQ ID NO: 296 - protein, 12 aa, Homo sapiens
  REGION 1..12: note = Description of sequence: gamma-gliadin
ADAPFPQPELPY

SEQ ID NO: 297 - protein, 12 aa, Homo sapiens
  REGION 1..12: note = Description of sequence: gamma-gliadin
QLQIFPQPELPY

SEQ ID NO: 298 - protein, 12 aa, Homo sapiens
  REGION 1..12: note = Description of sequence: gamma-gliadin
QLQPFPEPELPY

SEQ ID NO: 299 - protein, 12 aa, Homo sapiens
  REGION 1..12: note = Description of sequence: gamma-gliadin
QLQPFPQPEEPY

SEQ ID NO: 300 - protein, 15 aa, Homo sapiens
  REGION 1..15: note = Description of sequence: gamma-gliadin
ADAQPQPELPYPQPE

SEQ ID NO: 301 - protein, 14 aa, Homo sapiens
  REGION 1..14: note = Description of sequence: gamma-gliadin
ADAPQPELPYPQPE

SEQ ID NO: 302 - protein, 11 aa, Homo sapiens
  REGION 1..11: note = Description of sequence: gamma-gliadin
IQPELPYPQPE

SEQ ID NO: 303 - protein, 11 aa, Homo sapiens
  REGION 1..11: note = Description of sequence: gamma-gliadin
PQPELPEPQPE

SEQ ID NO: 304 - protein, 13 aa, Homo sapiens
  REGION 1..13: note = Description of sequence: gamma-gliadin
PFPQPELPYPQPE

SEQ ID NO: 305 - skipped

SEQ ID NO: 306 - protein, 12 aa, Homo sapiens
  REGION 1..12: note = Description of sequence: gamma-gliadin
FPQPELPYPQPE

SEQ ID NO: 307 - protein, 12 aa, Homo sapiens
  REGION 1..12: note = Description of sequence: gamma-gliadin
APQPELPYPQPE

SEQ ID NO: 308 - protein, 12 aa, Homo sapiens
  REGION 1..12: note = Description of sequence: gamma-gliadin
QPFPQPEQPFPW

SEQ ID NO: 309 - protein, 14 aa, Homo sapiens
  REGION 1..14: note = Description of sequence: gamma-gliadin
QPEPFPQPEQPFPW

SEQ ID NO: 310 - protein, 13 aa, Homo sapiens
  REGION 1..13: note = Description of sequence: gamma-gliadin
PEPFPQPEQPFPW

SEQ ID NO: 311 - protein, 12 aa, Homo sapiens
  REGION 1..12: note = Description of sequence: gamma-gliadin
EPFPQPEQPFPW

SEQ ID NO: 312 - protein, 11 aa, Homo sapiens
  REGION 1..11: note = Description of sequence: gamma-gliadin
PFPQPEQPFPW

SEQ ID NO: 313 - protein, 12 aa, Homo sapiens
  REGION 1..12: note = Description of sequence: gamma-gliadin
QLQPFPQPCLPY

SEQ ID NO: 314 - protein, 11 aa, Homo sapiens
  REGION 1..11: note = Description of sequence: gamma-gliadin
PQPELCYPQPE

SEQ ID NO: 315 - protein, 9 aa, synthetic construct
  REGION 1..9: note = Description of sequence: HA
YPYDVPDYA

SEQ ID NO: 316 - protein, 8 aa, synthetic construct
  REGION 1..8: note = Description of sequence: Strep
WSHPQFEK

SEQ ID NO: 317 - protein, 8 aa, synthetic construct
  REGION 1..8: note = Description of sequence: FLAG
DYKDDDDK

SEQ ID NO: 318 - protein, 10 aa, synthetic construct
  REGION 1..10: note = Description of sequence: c-myc
EQKLISEEDL

SEQ ID NO: 319 - protein, 5 aa, synthetic construct
  REGION 1..5: note = Description of sequence: motif
RYIRS

SEQ ID NO: 320 - protein, 4 aa, synthetic construct
  REGION 1..4: note = Description of sequence: motif
FHHT

SEQ ID NO: 321 - protein, 17 aa, synthetic construct
  REGION 1..17: note = Description of sequence: motif
WEAAAREACCRECCARA

SEQ ID NO: 322 - protein, 5 aa, synthetic construct
  REGION 1..5: note = Description of sequence: His-5
HHHHH

SEQ ID NO: 323 - protein, 6 aa, synthetic construct
  REGION 1..6: note = Description of sequence: His-6
HHHHHH

SEQ ID NO: 324 - protein, 188 aa, synthetic construct
  REGION 1..188: note = Description of sequence: DQB1*02:01 Sequence
RDSPCDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEW

SEQ ID NO: 325 - protein, 193 aa, synthetic construct
  REGION 1..193: note = Description of sequence: DQA1*05:01 Sequence
EDIVADHVASYGVNLYQSYGPSGQYTHEFDGDEQFYVDLGRKETVWKLPVLHQFRFDPQFALTNIAVLKHNLNILIKRSNSTCATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTE

SEQ ID NO: 326 - protein, 10 aa, synthetic construct
  REGION 1..10: note = Description of sequence: Linker
GGSAAAGGGG

SEQ ID NO: 327 - protein, 15 aa, synthetic construct
  REGION 1..15: note = Description of sequence: insulin (InsA 1-15 D4E)
GIVEQCCTSICSLYQ

SEQ ID NO: 328 - protein, 8 aa, Arachis hypogaea
  REGION 1..8: note = Description of sequence: peanut allergen
PGQFEDFF

SEQ ID NO: 329 - protein, 8 aa, Arachis hypogaea
  REGION 1..8: note = Description of sequence: peanut allergen
YLQGFSRN

SEQ ID NO: 330 - protein, 10 aa, Arachis hypogaea
  REGION 1..10: note = Description of sequence: peanut allergen
FNAEFNEIRR

SEQ ID NO: 331 - protein, 8 aa, Arachis hypogaea
  REGION 1..8: note = Description of sequence: peanut allergen
QEERGQRR

SEQ ID NO: 332 - protein, 10 aa, Arachis hypogaea
  REGION 1..10: note = Description of sequence: peanut allergen
DITNPINLRE

SEQ ID NO: 333 - protein, 10 aa, Arachis hypogaea
  REGION 1..10: note = Description of sequence: peanut allergen
NNFGKLFEVK

SEQ ID NO: 334 - protein, 6 aa, Arachis hypogaea
  REGION 1..6: note = Description of sequence: peanut allergen
GNLELV

SEQ ID NO: 335 - protein, 10 aa, Arachis hypogaea
  REGION 1..10: note = Description of sequence: peanut allergen
RRYTARLKEG

SEQ ID NO: 336 - protein, 10 aa, Arachis hypogaea
  REGION 1..10: note = Description of sequence: peanut allergen
ELHLLGFGIN

SEQ ID NO: 337 - protein, 10 aa, Arachis hypogaea
  REGION 1..10: note = Description of sequence: peanut allergen
HRIFLAGDKD

SEQ ID NO: 338 - protein, 10 aa, Arachis hypogaea
  REGION 1..10: note = Description of sequence: peanut allergen
IDQIEKQAKD

SEQ ID NO: 339 - protein, 10 aa, Arachis hypogaea
  REGION 1..10: note = Description of sequence: peanut allergen
KDLAFPGSGE

SEQ ID NO: 340 - protein, 10 aa, Arachis hypogaea
  REGION 1..10: note = Description of sequence: peanut allergen
KESHFVSARP

SEQ ID NO: 341 - protein, 25 aa, Arachis hypogaea
  REGION 1..25: note = Description of sequence: peanut allergen
NEGVIVKVSKEHVEELTKHAKSVSK

SEQ ID NO: 342 - protein, 10 aa, Arachis hypogaea
  REGION 1..10: note = Description of sequence: peanut allergen
HASARQQWEL

SEQ ID NO: 343 - protein, 10 aa, Arachis hypogaea
  REGION 1..10: note = Description of sequence: peanut allergen
QWELQGDRRC

SEQ ID NO: 344 - protein, 10 aa, Arachis hypogaea
  REGION 1..10: note = Description of sequence: peanut allergen
DRRCQSQLER

SEQ ID NO: 345 - protein, 10 aa, Arachis hypogaea
  REGION 1..10: note = Description of sequence: peanut allergen
LRPCEQHLMQ

SEQ ID NO: 346 - protein, 10 aa, Arachis hypogaea
  REGION 1..10: note = Description of sequence: peanut allergen
KIQRDEDSYE

SEQ ID NO: 347 - protein, 10 aa, Arachis hypogaea
  REGION 1..10: note = Description of sequence: peanut allergen
YERDPYSPSQ

SEQ ID NO: 348 - protein, 10 aa, Arachis hypogaea
  REGION 1..10: note = Description of sequence: peanut allergen
SQDPYSPSPY

SEQ ID NO: 349 - protein, 10 aa, Arachis hypogaea
  REGION 1..10: note = Description of sequence: peanut allergen
DRLQGRQQEQ

SEQ ID NO: 350 - protein, 10 aa, Arachis hypogaea
  REGION 1..10: note = Description of sequence: peanut allergen
KRELRNLPQQ

SEQ ID NO: 351 - protein, 10 aa, Arachis hypogaea
  REGION 1..10: note = Description of sequence: peanut allergen
QRCDLDVESG

SEQ ID NO: 352 - protein, 15 aa, Arachis hypogaea
  REGION 1..15: note = Description of sequence: peanut allergen
IETWNPNNQEFECAG

SEQ ID NO: 353 - protein, 15 aa, Arachis hypogaea
  REGION 1..15: note = Description of sequence: peanut allergen
GNIFSGFTPEFLAQA

SEQ ID NO: 354 - protein, 15 aa, Arachis hypogaea
  REGION 1..15: note = Description of sequence: peanut allergen
VTVRGGLRILSPDRK

SEQ ID NO: 355 - protein, 15 aa, Arachis hypogaea
  REGION 1..15: note = Description of sequence: peanut allergen
DEDEYEYDEEDRRRG

SEQ ID NO: 356 - protein, 12 aa, synthetic construct
  REGION 1..12: note = Description of sequence: MHC Class I alpha peptide epitope
APWIEQEAPEYW

SEQ ID NO: 357 - protein, 15 aa, synthetic construct
  REGION 1..15: note = Description of sequence: anchor modified MHC Class I alpha peptide epitope (
ADAAPWIEQEAPEYW

SEQ ID NO: 358 - protein, 13 aa, synthetic construct
  REGION 1..13: note = Description of sequence: Tim Grass peptide epitope
ESYKFIPALEAAV

SEQ ID NO: 359 - protein, 11 aa, synthetic construct
  REGION 1..11: note = Description of sequence: MAGE peptide epitope
ESEFQAALSRK

SEQ ID NO: 360 - protein, 15 aa, synthetic construct
  REGION 1..15: note = Description of sequence: NY-ESO1 peptide epitope
LKEFTVSGNILTIRL

SEQ ID NO: 361 - protein, 16 aa, synthetic construct
  REGION 1..16: note = Description of sequence: PMEL peptide epitope
WNRQLYPEWTEAQRLD

SEQ ID NO: 362 - protein, 17 aa, synthetic construct
  REGION 1..17: note = Description of sequence: HER2 peptide epitope
KVPIKWMALESILRRRF

SEQ ID NO: 363 - protein, 17 aa, synthetic construct
  REGION 1..17: note = Description of sequence: Tyrosinase peptide epitope
ALHIYMDGTMSQVQGSA

SEQ ID NO: 364 - protein, 15 aa, synthetic construct
  REGION 1..15: note = Description of sequence: Hepatitis C Virus (HCV) peptide epitope
GYKVLVLNPSVAATL

SEQ ID NO: 365 - protein, 20 aa, synthetic construct
  REGION 1..20: note = Description of sequence: HIV gag peptide epitope
AFSPEVIPMFSALSEGATPQ

SEQ ID NO: 366 - protein, 20 aa, synthetic construct
  REGION 1..20: note = Description of sequence: EBV-ENA2 peptide epitope
GQTYHLIVDTDSLGNPSLSV

SEQ ID NO: 367 - protein, 13 aa, synthetic construct
  REGION 1..13: note = Description of sequence: MOG peptide epitope
RTELLKDAIGEGK

SEQ ID NO: 368 - protein, 15 aa, synthetic construct
  REGION 1..15: note = Description of sequence: Type II Collagen alpha 1 chain (CII) peptide epitope
GIAGFKGEQGPKGEP

SEQ ID NO: 369 - protein, 15 aa, synthetic construct
  REGION 1..15: note = Description of sequence: Type II Collagen (CII) (259-273 G262A) peptide epitope
GIAAFKGEQGPKGEP

SEQ ID NO: 370 - protein, 641 aa, synthetic construct
  REGION 1..641: note = Description of sequence: MHC Class II Protein Construct
GGGGSGGGGSGGGGSPCNYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEWKAQGGGGSGGGGSGGGGSGGGGSGGGGSIKADHVSTYAAFVQTHRPTGEFMFEFDEDEMFYVDLDKKETVWHLEEFGQAFSFEAQGGLANIAILNNNLNTLIQRSNHTCATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQGGSAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 371 - protein, 665 aa, synthetic construct; identical to SEQ ID NO: 154
  REGION 1..665: note = Description of sequence: MHC Class II Protein Construct

SEQ ID NO: 372 - protein, 281 aa, Homo sapiens
  REGION 1..281: note = Description of sequence: Homo sapiens FasL
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 373 - protein, 335 aa, Homo sapiens
  REGION 1..335: note = Description of sequence: Fas receptor
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 374 - protein, 150 aa, Homo sapiens
  REGION 1..150: note = Description of sequence: FasL polypeptide
IGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYK

SEQ ID NO: 375 - protein, 179 aa, Homo sapiens
  REGION 1..179: note = Description of sequence: FasL polypeptide
QLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 376 - protein, 208 aa, Homo sapiens [sequence omitted]

SEQ ID NO: 377 - protein, 104 aa, Homo sapiens
  REGION 1..104: note = Description of sequence: IgV domain of a wt. human CD80
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSV

SEQ ID NO: 378 - protein, 220 aa, Homo sapiens
  REGION 1..220: note = Description of sequence: wt. CD28 polypeptide
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 379 - protein, 123 aa, Homo sapiens
  REGION 1..123: note = Description of sequence: wt. CD28 polypeptide
MLRLLLALNLFPSIQVTGNKILVKQSPMLVAYDNAVNLSWKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 380 - protein, 101 aa, Homo sapiens
  REGION 1..101: note = Description of sequence: wt. CD28
MLRLLLALNLFPSIQVTGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 381 - protein, 110 aa, Homo sapiens
  REGION 1..110: note = Description of sequence: wt. ectodomain human CD86
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVL

SEQ ID NO: 382 - protein, 110 aa, Homo sapiens; identical to SEQ ID NO: 381
  REGION 1..110: note = Description of sequence: IgV domain of a wt. human CD86

SEQ ID NO: 383 - protein, 8 aa, synthetic construct
  REGION 1..8: note = Description of sequence: Linker
GGSSGGSS

SEQ ID NO: 384 - protein, 12 aa, synthetic construct
  REGION 1..12: note = Description of sequence: Linker
GGSSGGSSGGSS

SEQ ID NO: 385 - protein, 16 aa, synthetic construct
  REGION 1..16: note = Description of sequence: Linker
GGSSGGSSGGSSGGSS

SEQ ID NO: 386 - protein, 20 aa, synthetic construct
  REGION 1..20: note = Description of sequence: Linker
GGSSGGSSGGSSGGSSGGSS

SEQ ID NO: 387 - protein, 24 aa, synthetic construct
  REGION 1..24: note = Description of sequence: Linker
GGSSGGSSGGSSGGSSGGSSGGSS

SEQ ID NO: 388 - protein, 28 aa, synthetic construct
  REGION 1..28: note = Description of sequence: Linker
GGSSGGSSGGSSGGSSGGSSGGSSGGSS

SEQ ID NO: 389 - protein, 32 aa, synthetic construct
  REGION 1..32: note = Description of sequence: Linker
GGSSGGSSGGSSGGSSGGSSGGSSGGSSGGSS

SEQ ID NO: 390 - protein, 36 aa, synthetic construct
  REGION 1..36: note = Description of sequence: Linker
GGSSGGSSGGSSGGSSGGSSGGSSGGSSGGSSGGSS

SEQ ID NO: 391 - protein, 40 aa, synthetic construct
  REGION 1..40: note = Description of sequence: Linker
GGSSGGSSGGSSGGSSGGSSGGSSGGSSGGSSGGSSGGSS

SEQ ID NO: 392 - protein, 10 aa, synthetic construct
  REGION 1..10: note = Description of sequence: Linker
GSGGSGSGGS

SEQ ID NO: 393 - protein, 15 aa, synthetic construct
  REGION 1..15: note = Description of sequence: Linker
GSGGSGSGGSGSGGS

SEQ ID NO: 394 - protein, 20 aa, synthetic construct
  REGION 1..20: note = Description of sequence: Linker
GSGGSGSGGSGSGGSGSGGS

SEQ ID NO: 395 - protein, 25 aa, synthetic construct
  REGION 1..25: note = Description of sequence: Linker
GSGGSGSGGSGSGGSGSGGSGSGGS

SEQ ID NO: 396 - protein, 30 aa, synthetic construct
  REGION 1..30: note = Description of sequence: Linker
GSGGSGSGGSGSGGSGSGGSGSGGSGSGGS

SEQ ID NO: 397 - protein, 35 aa, synthetic construct
  REGION 1..35: note = Description of sequence: Linker
GSGGSGSGGSGSGGSGSGGSGSGGSGSGGSGSGGS

SEQ ID NO: 398 - protein, 40 aa, synthetic construct
  REGION 1..40: note = Description of sequence: Linker
GSGGSGSGGSGSGGSGSGGSGSGGSGSGGSGSGGSGSGGS

SEQ ID NO: 399 - protein, 45 aa, synthetic construct
  REGION 1..45: note = Description of sequence: Linker
GSGGSGSGGSGSGGSGSGGSGSGGSGSGGSGSGGSGSGGSGSGGS

SEQ ID NO: 400 - protein, 50 aa, synthetic construct
  REGION 1..50: note = Description of sequence: Linker
GSGGSGSGGSGSGGSGSGGSGSGGSGSGGSGSGGSGSGGSGSGGSGSGGS

SEQ ID NO: 401 - protein, 8 aa, synthetic construct
  REGION 1..8: note = Description of sequence: Linker
GGGSGGGS

SEQ ID NO: 402 - protein, 12 aa, synthetic construct
  REGION 1..12: note = Description of sequence: Linker
GGGSGGGSGGGS

SEQ ID NO: 403 - protein, 16 aa, synthetic construct
  REGION 1..16: note = Description of sequence: Linker
GGGSGGGSGGGSGGGS

SEQ ID NO: 404 - protein, 20 aa, synthetic construct
  REGION 1..20: note = Description of sequence: Linker
GGGSGGGSGGGSGGGSGGGS

SEQ ID NO: 405 - protein, 24 aa, synthetic construct
  REGION 1..24: note = Description of sequence: Linker
GGGSGGGSGGGSGGGSGGGSGGGS

SEQ ID NO: 406 - protein, 28 aa, synthetic construct
  REGION 1..28: note = Description of sequence: Linker
GGGSGGGSGGGSGGGSGGGSGGGSGGGS

SEQ ID NO: 407 - protein, 32 aa, synthetic construct
  REGION 1..32: note = Description of sequence: Linker
GGGSGGGSGGGSGGGSGGGSGGGSGGGSGGGS

SEQ ID NO: 408 - protein, 36 aa, synthetic construct
  REGION 1..36: note = Description of sequence: Linker
GGGSGGGSGGGSGGGSGGGSGGGSGGGSGGGSGGGS

SEQ ID NO: 409 - protein, 40 aa, synthetic construct
  REGION 1..40: note = Description of sequence: Linker
GGGSGGGSGGGSGGGSGGGSGGGSGGGSGGGSGGGSGGGS

SEQ ID NO: 410 - protein, 8 aa, synthetic construct
  REGION 1..8: note = Description of sequence: Linker
GGSGGGSG

SEQ ID NO: 411 - protein, 12 aa, synthetic construct
  REGION 1..12: note = Description of sequence: Linker
GGSGGGSGGGSG

SEQ ID NO: 412 - protein, 16 aa, synthetic construct
  REGION 1..16: note = Description of sequence: Linker
GGSGGGSGGGSGGGSG

SEQ ID NO: 413 - protein, 20 aa, synthetic construct
  REGION 1..20: note = Description of sequence: Linker
GGSGGGSGGGSGGGSGGGSG

SEQ ID NO: 414 - protein, 24 aa, synthetic construct
  REGION 1..24: note = Description of sequence: Linker
GGSGGGSGGGSGGGSGGGSGGGSG

SEQ ID NO: 415 - protein, 28 aa, synthetic construct
  REGION 1..28: note = Description of sequence: Linker
GGSGGGSGGGSGGGSGGGSGGGSGGGSG

SEQ ID NO: 416 - protein, 32 aa, synthetic construct
  REGION 1..32: note = Description of sequence: Linker
GGSGGGSGGGSGGGSGGGSGGGSGGGSGGGSG

SEQ ID NO: 417 - protein, 36 aa, synthetic construct
  REGION 1..36: note = Description of sequence: Linker
GGSGGGSGGGSGGGSGGGSGGGSGGGSGGGSGGGSG

SEQ ID NO: 418 - protein, 40 aa, synthetic construct
  REGION 1..40: note = Description of sequence: Linker
GGSGGGSGGGSGGGSGGGSGGGSGGGSGGGSGGGSGGGSG

SEQ ID NO: 419 - protein, 10 aa, synthetic construct
  REGION 1..10: note = Description of sequence: Linker
GGSGGGGSGG

SEQ ID NO: 420 - protein, 15 aa, synthetic construct
  REGION 1..15: note = Description of sequence: Linker
GGSGGGGSGGGGSGG

SEQ ID NO: 421 - protein, 20 aa, synthetic construct
  REGION 1..20: note = Description of sequence: Linker
GGSGGGGSGGGGSGGGGSGG

SEQ ID NO: 422 - protein, 25 aa, synthetic construct
  REGION 1..25: note = Description of sequence: Linker
GGSGGGGSGGGGSGGGGSGGGGSGG

SEQ ID NO: 423 - protein, 30 aa, synthetic construct
  REGION 1..30: note = Description of sequence: Linker
GGSGGGGSGGGGSGGGGSGGGGSGGGGSGG

SEQ ID NO: 424 - protein, 35 aa, synthetic construct
  REGION 1..35: note = Description of sequence: Linker
GGSGGGGSGGGGSGGGGSGGGGSGGGGSGGGGSGG

SEQ ID NO: 425 - protein, 40 aa, synthetic construct
  REGION 1..40: note = Description of sequence: Linker
GGSGGGGSGGGGSGGGGSGGGGSGGGGSGGGGSGGGGSGG

SEQ ID NO: 426 - protein, 45 aa, synthetic construct
  REGION 1..45: note = Description of sequence: Linker
GGSGGGGSGGGGSGGGGSGGGGSGGGGSGGGGSGGGGSGGGGSGG

SEQ ID NO: 427 - protein, 50 aa, synthetic construct
  REGION 1..50: note = Description of sequence: Linker
GGSGGGGSGGGGSGGGGSGGGGSGGGGSGGGGSGGGGSGGGGSGGGGSGG

SEQ ID NO: 428 - protein, 10 aa, synthetic construct
  REGION 1..10: note = Description of sequence: Linker
GSGSGGSGSG

SEQ ID NO: 429 - protein, 15 aa, synthetic construct
  REGION 1..15: note = Description of sequence: Linker
GSGSGGSGSGGSGSG

SEQ ID NO: 430 - protein, 20 aa, synthetic construct
  REGION 1..20: note = Description of sequence: Linker
GSGSGGSGSGGSGSGGSGSG

SEQ ID NO: 431 - protein, 25 aa, synthetic construct
  REGION 1..25: note = Description of sequence: Linker
GSGSGGSGSGGSGSGGSGSGGSGSG

SEQ ID NO: 432 - protein, 30 aa, synthetic construct
  REGION 1..30: note = Description of sequence: Linker
GSGSGGSGSGGSGSGGSGSGGSGSGGSGSG

SEQ ID NO: 433 - protein, 35 aa, synthetic construct
  REGION 1..35: note = Description of sequence: Linker
GSGSGGSGSGGSGSGGSGSGGSGSGGSGSGGSGSG

SEQ ID NO: 434 - protein, 40 aa, synthetic construct
  REGION 1..40: note = Description of sequence: Linker
GSGSGGSGSGGSGSGGSGSGGSGSGGSGSGGSGSGGSGSG

SEQ ID NO: 435 - protein, 45 aa, synthetic construct
  REGION 1..45: note = Description of sequence: Linker
GSGSGGSGSGGSGSGGSGSGGSGSGGSGSGGSGSGGSGSGGSGSG

SEQ ID NO: 436 - protein, 50 aa, synthetic construct
  REGION 1..50: note = Description of sequence: Linker
GSGSGGSGSGGSGSGGSGSGGSGSGGSGSGGSGSGGSGSGGSGSGGSGSG

SEQ ID NO: 437 - protein, 10 aa, synthetic construct
  REGION 1..10: note = Description of sequence: Linker
GSGGGGSGGG

SEQ ID NO: 438 - protein, 15 aa, synthetic construct
  REGION 1..15: note = Description of sequence: Linker
GSGGGGSGGGGSGGG

SEQ ID NO: 439 - protein, 20 aa, synthetic construct
  REGION 1..20: note = Description of sequence: Linker
GSGGGGSGGGGSGGGGSGGG

SEQ ID NO: 440 - protein, 25 aa, synthetic construct
  REGION 1..25: note = Description of sequence: Linker
GSGGGGSGGGGSGGGGSGGGGSGGG

SEQ ID NO: 441 - protein, 30 aa, synthetic construct
  REGION 1..30: note = Description of sequence: Linker
GSGGGGSGGGGSGGGGSGGGGSGGGGSGGG

SEQ ID NO: 442 - protein, 35 aa, synthetic construct
  REGION 1..35: note = Description of sequence: Linker
GSGGGGSGGGGSGGGGSGGGGSGGGGSGGGGSGGG

SEQ ID NO: 443 - protein, 40 aa, synthetic construct
  REGION 1..40: note = Description of sequence: Linker
GSGGGGSGGGGSGGGGSGGGGSGGGGSGGGGSGGGGSGGG

SEQ ID NO: 444 - protein, 45 aa, synthetic construct
  REGION 1..45: note = Description of sequence: Linker
GSGGGGSGGGGSGGGGSGGGGSGGGGSGGGGSGGGGSGGGGSGGG

SEQ ID NO: 445 - protein, 50 aa, synthetic construct
  REGION 1..50: note = Description of sequence: Linker
GSGGGGSGGGGSGGGGSGGGGSGGGGSGGGGSGGGGSGGGGSGGGGSGGG

SEQ ID NO: 446 - protein, 10 aa, synthetic construct
  REGION 1..10: note = Description of sequence: Linker
GGGSGGGGSG

SEQ ID NO: 447 - protein, 15 aa, synthetic construct
  REGION 1..15: note = Description of sequence: Linker
GGGSGGGGSGGGGSG

SEQ ID NO: 448 - protein, 20 aa, synthetic construct
  REGION 1..20: note = Description of sequence: Linker
GGGSGGGGSGGGGSGGGGSG

SEQ ID NO: 449 - protein, 25 aa, synthetic construct
  REGION 1..25: note = Description of sequence: Linker
GGGSGGGGSGGGGSGGGGSGGGGSG

SEQ ID NO: 450 - protein, 30 aa, synthetic construct
  REGION 1..30: note = Description of sequence: Linker
GGGSGGGGSGGGGSGGGGSGGGGSGGGGSG

SEQ ID NO: 451 - protein, 35 aa, synthetic construct
  REGION 1..35: note = Description of sequence: Linker
GGGSGGGGSGGGGSGGGGSGGGGSGGGGSGGGGSG

SEQ ID NO: 452 - protein, 40 aa, synthetic construct
  REGION 1..40: note = Description of sequence: Linker
GGGSGGGGSGGGGSGGGGSGGGGSGGGGSGGGGSGGGGSG

SEQ ID NO: 453 - protein, 45 aa, synthetic construct
  REGION 1..45: note = Description of sequence: Linker
GGGSGGGGSGGGGSGGGGSGGGGSGGGGSGGGGSGGGGSGGGGSG

SEQ ID NO: 454 - protein, 50 aa, synthetic construct
  REGION 1..50: note = Description of sequence: Linker
GGGSGGGGSGGGGSGGGGSGGGGSGGGGSGGGGSGGGGSGGGGSGGGGSG

SEQ ID NO: 455 - protein, 10 aa, synthetic construct
  REGION 1..10: note = Description of sequence: Linker
GSSSGGSSSG

SEQ ID NO: 456 - protein, 15 aa, synthetic construct
  REGION 1..15: note = Description of sequence: Linker
GSSSGGSSSGGSSSG

SEQ ID NO: 457 - protein, 20 aa, synthetic construct
  REGION 1..20: note = Description of sequence: Linker
GSSSGGSSSGGSSSGGSSSG

SEQ ID NO: 458 - protein, 25 aa, synthetic construct
  REGION 1..25: note = Description of sequence: Linker
GSSSGGSSSGGSSSGGSSSGGSSSG

SEQ ID NO: 459 - protein, 30 aa, synthetic construct
  REGION 1..30: note = Description of sequence: Linker
GSSSGGSSSGGSSSGGSSSGGSSSGGSSSG

SEQ ID NO: 460 - protein, 35 aa, synthetic construct
  REGION 1..35: note = Description of sequence: Linker
GSSSGGSSSGGSSSGGSSSGGSSSGGSSSGGSSSG

SEQ ID NO: 461 - protein, 40 aa, synthetic construct
  REGION 1..40: note = Description of sequence: Linker
GSSSGGSSSGGSSSGGSSSGGSSSGGSSSGGSSSGGSSSG

SEQ ID NO: 462 - protein, 45 aa, synthetic construct
  REGION 1..45: note = Description of sequence: Linker
GSSSGGSSSGGSSSGGSSSGGSSSGGSSSGGSSSGGSSSGGSSSG

SEQ ID NO: 463 - protein, 50 aa, synthetic construct
  REGION 1..50: note = Description of sequence: Linker
GSSSGGSSSGGSSSGGSSSGGSSSGGSSSGGSSSGGSSSGGSSSGGSSSG

SEQ ID NO: 464 - protein, 10 aa, synthetic construct
  REGION 1..10: note = Description of sequence: Linker
GSSSSGSSSS

SEQ ID NO: 465 - protein, 15 aa, synthetic construct
  REGION 1..15: note = Description of sequence: Linker
GSSSSGSSSSGSSSS

SEQ ID NO: 466 - protein, 20 aa, synthetic construct
  REGION 1..20: note = Description of sequence: Linker
GSSSSGSSSSGSSSSGSSSS

SEQ ID NO: 467 - protein, 25 aa, synthetic construct
  REGION 1..25: note = Description of sequence: Linker
GSSSSGSSSSGSSSSGSSSSGSSSS

SEQ ID NO: 468 - protein, 30 aa, synthetic construct
  REGION 1..30: note = Description of sequence: Linker
GSSSSGSSSSGSSSSGSSSSGSSSSGSSSS

SEQ ID NO: 469 - protein, 35 aa, synthetic construct
  REGION 1..35: note = Description of sequence: Linker
GSSSSGSSSSGSSSSGSSSSGSSSSGSSSSGSSSS

SEQ ID NO: 470 - protein, 40 aa, synthetic construct
  REGION 1..40: note = Description of sequence: Linker
GSSSSGSSSSGSSSSGSSSSGSSSSGSSSSGSSSSGSSSS

SEQ ID NO: 471 - protein, 45 aa, synthetic construct
  REGION 1..45: note = Description of sequence: Linker
GSSSSGSSSSGSSSSGSSSSGSSSSGSSSSGSSSSGSSSSGSSSS

SEQ ID NO: 472 - protein, 50 aa, synthetic construct
  REGION 1..50: note = Description of sequence: Linker
GSSSSGSSSSGSSSSGSSSSGSSSSGSSSSGSSSSGSSSSGSSSSGSSSS

SEQ ID NO: 473 - protein, 10 aa, synthetic construct
  REGION 1..10: note = Description of sequence: Linker
GGGGSGGGGS

SEQ ID NO: 474 - protein, 15 aa, synthetic construct
  REGION 1..15: note = Description of sequence: Linker
GGGGSGGGGSGGGGS

SEQ ID NO: 475 - protein, 20 aa, synthetic construct
  REGION 1..20: note = Description of sequence: Linker
GGGGSGGGGSGGGGSGGGGS

SEQ ID NO: 476 - protein, 25 aa, synthetic construct
  REGION 1..25: note = Description of sequence: Linker
GGGGSGGGGSGGGGSGGGGSGGGGS

SEQ ID NO: 477 - protein, 30 aa, synthetic construct
  REGION 1..30: note = Description of sequence: Linker
GGGGSGGGGSGGGGSGGGGSGGGGSGGGGS

SEQ ID NO: 478 - protein, 35 aa, synthetic construct
  REGION 1..35: note = Description of sequence: Linker
GGGGSGGGGSGGGGSGGGGSGGGGSGGGGSGGGGS

SEQ ID NO: 479 - protein, 40 aa, synthetic construct
  REGION 1..40: note = Description of sequence: Linker
GGGGSGGGGSGGGGSGGGGSGGGGSGGGGSGGGGSGGGGS

SEQ ID NO: 480 - protein, 45 aa, synthetic construct
  REGION 1..45: note = Description of sequence: Linker
GGGGSGGGGSGGGGSGGGGSGGGGSGGGGSGGGGSGGGGSGGGGS

SEQ ID NO: 481 - protein, 10 aa, synthetic construct
  REGION 1..10: note = Description of sequence: Linker
AAAGGAAAGG

SEQ ID NO: 482 - protein, 15 aa, synthetic construct
  REGION 1..15: note = Description of sequence: Linker
AAAGGAAAGGAAAGG

SEQ ID NO: 483 - protein, 20 aa, synthetic construct
  REGION 1..20: note = Description of sequence: Linker
AAAGGAAAGGAAAGGAAAGG

SEQ ID NO: 484 - protein, 25 aa, synthetic construct
  REGION 1..25: note = Description of sequence: Linker
AAAGGAAAGGAAAGGAAAGGAAAGG

SEQ ID NO: 485 - protein, 30 aa, synthetic construct
  REGION 1..30: note = Description of sequence: Linker
AAAGGAAAGGAAAGGAAAGGAAAGGAAAGG

SEQ ID NO: 486 - protein, 35 aa, synthetic construct
  REGION 1..35: note = Description of sequence: Linker
AAAGGAAAGGAAAGGAAAGGAAAGGAAAGGAAAGG

SEQ ID NO: 487 - protein, 40 aa, synthetic construct
  REGION 1..40: note = Description of sequence: Linker
AAAGGAAAGGAAAGGAAAGGAAAGGAAAGGAAAGGAAAGG

SEQ ID NO: 488 - protein, 45 aa, synthetic construct
  REGION 1..45: note = Description of sequence: Linker
AAAGGAAAGGAAAGGAAAGGAAAGGAAAGGAAAGGAAAGGAAAGG

SEQ ID NO: 489 - protein, 50 aa, synthetic construct
  REGION 1..50: note = Description of sequence: Linker
AAAGGAAAGGAAAGGAAAGGAAAGGAAAGGAAAGGAAAGGAAAGGAAAGG

SEQ ID NO: 490 - protein, 16 aa, synthetic construct
  REGION 1..16: note = Description of sequence: Linker
GGSAAAGGGGSAAAGG

SEQ ID NO: 491 - protein, 24 aa, synthetic construct
  REGION 1..24: note = Description of sequence: Linker
GGSAAAGGGGSAAAGGGGSAAAGG

SEQ ID NO: 492 - protein, 32 aa, synthetic construct
  REGION 1..32: note = Description of sequence: Linker
GGSAAAGGGGSAAAGGGGSAAAGGGGSAAAGG

SEQ ID NO: 493 - protein, 40 aa, synthetic construct
  REGION 1..40: note = Description of sequence: Linker
GGSAAAGGGGSAAAGGGGSAAAGGGGSAAAGGGGSAAAGG

SEQ ID NO: 494 - protein, 48 aa, synthetic construct
  REGION 1..48: note = Description of sequence: Linker
GGSAAAGGGGSAAAGGGGSAAAGGGGSAAAGGGGSAAAGGGGSAAAGG

SEQ ID NO: 495 - protein, 56 aa, synthetic construct
  REGION 1..56: note = Description of sequence: Linker
GGSAAAGGGGSAAAGGGGSAAAGGGGSAAAGGGGSAAAGGGGSAAAGGGGSAAAGG

SEQ ID NO: 496 - protein, 64 aa, synthetic construct
  REGION 1..64: note = Description of sequence: Linker
GGSAAAGGGGSAAAGGGGSAAAGGGGSAAAGGGGSAAAGGGGSAAAGGGGSAAAGGGGSAAAGG

SEQ ID NO: 497 - protein, 72 aa, synthetic construct
  REGION 1..72: note = Description of sequence: Linker
GGSAAAGGGGSAAAGGGGSAAAGGGGSAAAGGGGSAAAGGGGSAAAGGGGSAAAGGGGSAAAGGGGSAAAGG

SEQ ID NO: 498 - protein, 80 aa, synthetic construct
  REGION 1..80: note = Description of sequence: Linker
GGSAAAGGGGSAAAGGGGSAAAGGGGSAAAGGGGSAAAGGGGSAAAGGGGSAAAGGGGSAAAGGGGSAAAGGGGSAAAGG

SEQ ID NO: 499 - protein, 441 aa, synthetic construct
  REGION 1..441: note = Description of sequence: MHC DQ2.5 construct
  REGION 199..248: note = This region depicts a section of repeats, any conbination of amino acids may be present, for instance, the sequence GGGGS maybe be repeated form 0 to 10 times
  VARIANT 199..248: note = These amino acids may be absent
  DISULFID 5..331: note = Cysteine bond
  VARIANT 199..248: note = X resprents any known amino acid; for instance, S or G or an GGGGS linker
RDSPCDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKXXXXXXXXXXXXXXXXXXXXXXXXXXXXXXXXXXXXXXXXXXXXXXXXXXEDIVADHVASYGVNLYQSYGPSGQYTHEFDGDEQFYVDLGRKETVWKLPVLRQFRFDPQFALTNIAVLKHNLNSLIKRSNSTCATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTE

SEQ ID NO: 500 - protein, 419 aa, synthetic construct
  REGION 1..419: note = Description of sequence: MHC DQ2.5 construct
  REGION 189..238: note = This region depicts a section of repeats, any conbination of amino acids may be present, for instance, the sequence GGGGS maybe be repeated form 0 to 10 times
  VARIANT 189..238: note = X resprents any known amino acid; for instance, S or G or an GGGGS linker
  VARIANT 189..238: note = These amino acids may be absent
  DISULFID 5..322: note = Cysteine bond
RDSPCDFVYQFKGMCYFTNGTERVRLVTRYIYNREEYARFDSDVGVYRAVTPLGPPAAEYWNSQKEVLERTRAELDTVCRHNYQLELRTTLQRRVEPTVTISPSRTEALNHHNLLVCSVTDFYPAQIKVRWFRNDQEETTGVVSTPLIRNGDWTFQILVMLEMTPQRGDVYTCHVEHPSLQNPIIVEWXXXXXXXXXXXXXXXXXXXXXXXXXXXXXXXXXXXXXXXXXXXXXXXXXXEDIVADHVASYGVNLYQSYGPSGQYSHEFDGDEEFYVDLERKETVWQLPLFRRFRRFDPQFALTNIAVLKHNLNIVIKRSNSTCATNEVPEVTVFSKSPVTLGQPNTLICLVDNIFPPVVNITWLSNGHSVTEGVSETSFLSKSDHSFFKISYLTFLPSDDEIYDCKVEHWGLDEPLLKHW